(19) United States
(12) Patent Application Publication
(10) Pub. No.: US 2026/0021192 A1
(43) Pub. Date: Jan. 22, 2026

(54) PROTEINS HAVING A COVALENT WARHEAD

(71) Applicant: Topaz Therapeutics, Inc., San Francisco, CA (US)

(72) Inventors: Tony Pisal Tang, Lafayette, CO (US); Aaron J. Cantor, San Francisco, CA (US); Seth H. Cassel, Brookline, MA (US); Eliot L. Coffey, San Carlos, CA (US); Thomas C. Harding, San Francisco, CA (US); Lucas Y.-L. Liu, San Francisco, CA (US); Minh Nguyen, San Francisco, CA (US); Nektaria Petronikolou, San Francisco, CA (US); Saathvika Ravi, San Francisco, CA (US); Krithica Sanjana, San Francisco, CA (US); Stan Yoo, San Francisco, CA (US)

(21) Appl. No.: 19/273,053

(22) Filed: Jul. 17, 2025

Related U.S. Application Data
(60) Provisional application No. 63/672,421, filed on Jul. 17, 2024.

(51) Int. Cl. A61K 47/68; A61K 51/10; C07K 16/28; C40B 30/04

(52) U.S. Cl. CPC A61K 47/6803; A61K 47/6849; A61K 47/6889; A61K 51/103; A61K 51/1093; C07K 16/2863; C40B 30/04; A61K 2121/00

(57) ABSTRACT

The present disclosure relates to proteins having a covalent warhead and compositions of same, as well as methods of making and using the proteins . . . .

### CROSS REFERENCE TO RELATED APPLICATIONS

This application claims the benefit of U.S. Provisional Application No. 63/672,421, filed on Jul. 17, 2024, which is incorporated by reference in its entirety.

### SEQUENCE LISTING

This application contains a Sequence Listing that has been submitted electronically as an XML file named 57891-0006W01_SL_ST26.xml. The XML file, created on Jul. 16, 2025, is 44,670 bytes in size. The material in the XML file is hereby incorporated by reference in its entirety.

### FIELD

The disclosure relates to the technical fields of immunology, chemistry, and medicine.

### BACKGROUND

Antibody drug conjugates (ADCs) are a class of agents that include an antibody and a cytotoxic payload attached to the antibody via a chemical linker. Despite the increasing number of ADCs, many ADCs have not demonstrated a therapeutic benefit as compared to controls. Additional compositions and methods for inhibiting a target protein or inducing cytotoxicity of a mammalian cell expressing a target protein are needed.

### SUMMARY

Provided herein are proteins having a covalent warhead and compositions comprising the same, as well as methods of making and using the proteins and compositions, kits comprising the protein or composition, and methods of screening.

This disclosure also features biomolecules (e.g., a macromolecule, such as a polypeptide or a protein; or a smaller building block thereof (e.g., a peptide or an individual amino acid)), having one or more reactive groups (e.g., electrophilic reactive groups) e.g. a covalent warhead, compositions containing the same, as well as methods of making and using the proteins and compositions, kits comprising the protein or composition, and methods of screening.

This disclosure also features chemical entities that are useful for preparing the biomolecules described herein. Said chemical entities include, but are not limited to, dually reactive spacer groups, e.g. chemical entities having the general formula (A) shown below:

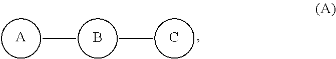

Each of A and C is an independently selected electrophilic chemical moieties that can form one or more covalent bonds with one or more nucleophilic groups, e.g., nucleophilic groups that are typically present in a biomolecule, e.g., an optionally substituted amino (—NH₂) group or a thiol (—SH) group. Said electrophilic chemical moieties described above are sometimes referred to herein as “reactive groups” or “convalent warheads.” B is an optional spacer group (typically an organic moiety), of a desired length, that covalently connects A and C to one another. Examples of A and C include, without limitation, flurosulfates, (heterocyclyl)sulfates, hydroxyamines, azides, Au(III)-complexed aryl (e.g., phenyl) rings, and maleimides. For ease of reference, formula (A) chemical entities are sometimes referred to herein as “linkers.” Some non-limiting examples of formula (A) chemical entities are provided throughout this specification, and for illustrative purposes only, are provided below.

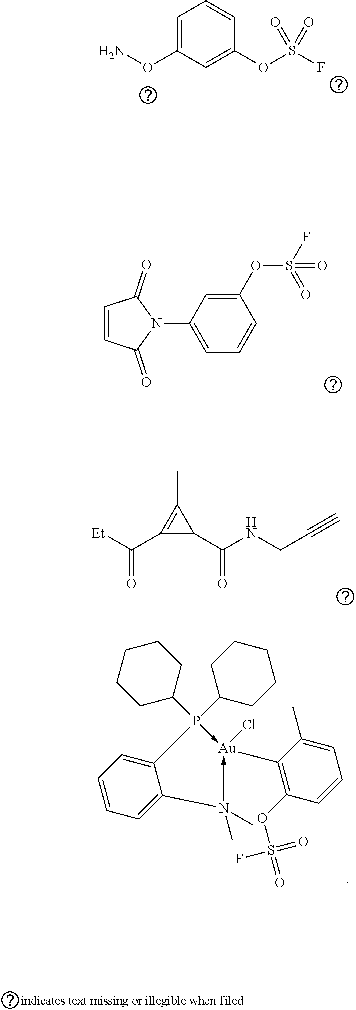

In one aspect, this disclosure features gold-containing organometallic agents (gold is also referred to herein by its atomic symbol “Au”), e.g., Au(III). The gold-containing organometallic agents described herein include, without limitation, gold complexes, e.g., Au(III) complexes, e.g., gold cyclometalated complexes, e.g., Au(III) cyclometalated complexes. The organometallic agents described herein can be used, for example, to (selectively) modify a biomolecule bearing one or more nucleophilic functional groups. Exemplary nucleophilic functional groups include protic functional groups, e.g., a thiol group, e.g. a thiol group that is associated with a cysteine. While not wishing to be bound by theory, it is believed that the organometallic agents described herein form covalent linkages with the one or more of the nucleophilic groups present on the biomolecule, thereby modifying the biomolecule.

Accordingly, in another aspect, this disclosure features methods of modifying a biomolecule, e.g., a macromolecule, such as a polypeptide or a protein; or a smaller building block thereof (e.g., a peptide or an individual amino acid). The methods include contacting the biomolecule with a gold-containing organometalic agent described herein. In some embodiments, modifying includes arylating a nucleophilic functional group, e.g., a protic functional group, e.g., a thiol group that is present on the biomolecule. In certain embodiments, the thiol group is associated with a cysteine.

In one aspect, this disclosure features compounds having formula (I):

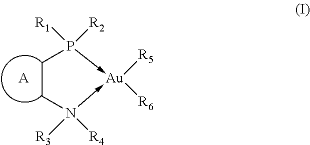

wherein:
Ring A is:
   C₆₋₁₄ aryl optionally substituted with 1-4 substituents independently selected from the group consisting of oxo, R^{a}, and R^{b}; or
   heteroaryl of 5-12 ring atoms, wherein 1-4 ring atoms are heteroatoms, each independently selected from the group consisting of N, N(H), N(R^{c}), O, and S(O)₀₋₂, and wherein the heteroaryl is optionally substituted with 1-4 substituents independently selected from the group consisting of oxo, R^{a}, and R^{b};
each of R¹ and R² is independently selected from the group consisting of:
   C₆₋₁₄ aryl optionally substituted with 1-4 substituents independently selected from the group consisting of oxo, R^{a}, and R^{b}; and
   C₃₋₁₂ cycloalkyl optionally substituted with substituents independently selected from the group consisting of oxo, R^{a}, and R^{b};
each of R³ and R⁴ is independently selected from the group consisting of:
   C₁₋₁₀ alkyl optionally substituted with 1-4 independently selected R^{d}; and
   C₃₋₁₂ cycloalkyl optionally substituted with substituents independently selected from the group consisting of oxo, R^{a}, and R^{b};
R⁵ is a coordinating anion;
R⁶ is —*R⁶¹-R⁶²-R⁶³; wherein, the * indicates the point of attachment of R⁶¹-R⁶²-R⁶³ to Au:
R⁶¹ is:
   divalent C₆-10 aryl optionally substituted with 1-4 substituents independently selected from the group consisting of oxo, R^{a}, and R^{b}; or
   divalent heteroaryl of 5-10 ring atoms, wherein 1-4 ring atoms are heteroatoms, each independently selected from the group consisting of N, N(H), N(R^{c}), O, and S(O)₀₋₂, and wherein the heteroaryl is optionally substituted with 1-4 substituents independently selected from the group consisting of oxo, R^{a}, and R^{b};
R⁶² is absent or is C₁-C₁₆ alkylene, C₂-C₁₆ alkenylene, or C₂-C₁₆ alkynylene, each of which is optionally substituted with 1-2 substituents independently selected from halogen and C₃-C₆ cycloalkyl; and wherein 1-8 alkylene units are optionally replaced with a group independently selected from the group consisting of:
(i) —O—;
(ii) —NH—;
(iii) —N(C₁-C₆ alkyl)-;
(iv) —C(O)—;
(v) —S—;
(vi) —S(O)—;
(vii) —S(O)₂—;
(viii) divalent C₃-C₁₀ cycloalkyl, which is optionally substituted with 1-4 R^{a}
(ix) divalent C₆-C₁₀ aryl, which is optionally substituted with 1-4 R^{a};
(x) divalent heteroaryl of 5-10 ring atoms, wherein 1-4 ring atoms are heteroatoms, each independently selected from the group consisting of N, N(H), N(R^{c}), O, and S(O)₀₋₂; and which is optionally substituted with 1-4 R^{a}; and
(xi) divalent heterocyclyl of 3-10 ring atoms, wherein 1-3 ring atoms are heteroatoms, each independently selected from the group consisting of N, N(H), N(R^{c}), N(O⁻), O, and S(O)₀₋₂, and which is optionally substituted with 1-4 R^{a};
R⁶³ is a reactive group;
each occurrence of R^{a} is independently selected from the group consisting of: halo; cyano; C₁₋₁₀ alkyl which is optionally substituted with 1-6 independently selected R^{d}; C₂₋₆ alkenyl; C₂₋₆ alkynyl; C₁₋₄ alkoxy; C₁₋₄ haloalkoxy; —S(O)₁₋₂(C₁₋₄ alkyl); —S(O)(═NH)(C₁₋₄ alkyl); —NR^{e}R^{f}; —OH; —S(O)₁₋₂NR′R″; —C₁₋₄ thioalkoxy; —NO₂; —C(═O)(C₁₋₁₀ alkyl); —C(═O)O(C₁₋₄ alkyl); —OC(═O)(C₁₋₄ alkyl); —C(═O)OH; —C(═O)NR′R″; —NR′C(═O)(C₁₋₄ alkyl) and —SF₅;
each occurrence of R^{b} is independently selected from the group consisting of:
   L¹-C₃₋₁₂ cycloalkyl or C₃₋₁₂ cycloalkenyl, each of which is optionally substituted with 1-4 substituents independently selected from the group consisting of oxo and R^{d};
   L¹-heterocyclyl or L¹-heterocycloalkenyl of 3-12 ring atoms, wherein 1-3 ring atoms are heteroatoms, each independently selected from the group consisting of N, N(H), N(R^{c}), O, and S(O)₀₋₂, and wherein the heterocyclyl or heterocycloalkenyl is optionally substituted with 1-4 substituents independently selected from the group consisting of oxo and R^{d};
   L¹-heteroaryl of 5-12 ring atoms, wherein 1-4 ring atoms are heteroatoms, each independently selected from the group consisting of N, N(H), N(R^{c}), O, and S(O)₀₋₂, and wherein the heteroaryl is optionally substituted with 1-4 substituents independently selected from the group consisting of oxo and R^{d}; and
   L¹-C₆₋₁₀ aryl optionally substituted with 1-4 substituents independently selected from the group consisting of oxo and R^{d};
L¹ is a bond or C₁₋₄ alkylene;
each occurrence of R^{e} is independently selected from the group consisting of: C₁₋₆ alkyl optionally substituted with 1-3 independently selected R^{a}; —C(O)(C₁₋₄ alkyl); —C(O)O(C₁₋₄ alkyl); —CONR′R″; —S(O)₁₋₂NR′R″; —S(O)₁₋₂(C₁₋₄ alkyl); —OH; and C₁₋₄ alkoxy;
each occurrence of R^{d} is independently selected from the group consisting of: —OH; -halo; —NR^{e}R^{f}; C₁₋₄ alkoxy; C₁₋₄ haloalkoxy; —C(═O)O(C₁₋₄ alkyl); —C(═O)(C₁₋₄ alkyl); —OC(═O)(C₁₋₄ alkyl); —C(═O)OH; —CONR′R″; —S(O)₁₋₂NR′R″; —S(O)₁₋₂(C₁₋₄ alkyl); and cyano;
each occurrence of R^{e} and R^{f} is independently selected from the group consisting of: H; C₁₋₆ alkyl optionally substituted with 1-3 substituents each independently selected from the group consisting of NR′R″, —OH, halo, C₁₋₄ alkoxy, and C₁₋₄ haloalkoxy; —C(O)(C₁₋₄ alkyl); —C(O)O(C₁₋₄ alkyl); —CONR′R″; —S(O)₁₋₂NR′R″; —S(O)₁₋₂(C₁₋₄ alkyl); —OH; and C₁₋₄ alkoxy; and
each occurrence of R′ and R″ is independently selected from the group consisting of: H; —OH; and C₁₋₄ alkyl.

In a further aspect, this disclosure features a comnposidon that includes a thiol containing biomolecule (e.g., a polypeptide or a protein) and a compound of Formula (I).

In a further aspect, this disclosure features a composition that includes a biomolecule having at least one cysteine residue and a compound ofFormula (I).

In a further aspect, this disclosure features a method for preparing a thiol-aryl conjugated biomolecule, the method comprising contacting a compound of Formula (I) with a biomolecule that includes at least one thiol group under conditions sufficient to prepare the thiol-aryl conjugated biomolecule. The biomolecules can optionally include one or more reactive groups as described herein.

In a further aspect, this disclosure features methods of preparing a cysteine-aryl conjugated biomolecule, the method comprising contacting a compound of Formula (I) with a biomolecule that includes at least one cysteine residue under conditions sufficient to prepare the cysteine-aryl conjugated biomolecule. The biomolecules can optionally include one or more reactive groups as described herein.

In a further aspect, this disclosure features methods of preparing a gold(III) aryl complex comprising contacting a compound of Formula (I) with an aryl halide under conditions sufficient to prepare the gold(III) aryl complex. In certain embodiments, the aryl halide is an aryl iodide.

Provided herein are proteins having a covalent warhead and compositions of same, as well as methods of making and using the proteins and compositions, kits comprising the protein or composition, and methods of screening.

Some embodiments provide a protein comprising an antigen-binding domain, wherein the antigen-binding domain comprising an oxime, the oxime having the structure:

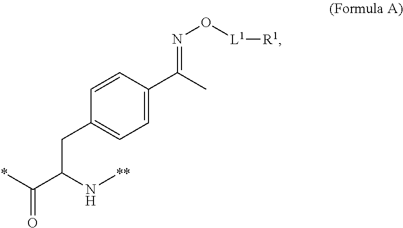

wherein:
* and ** represent the points of connection of the oxime to the antigen-binding domain;
L¹ is a bond or a C1-C6 alkylene optionally substituted with 1-2 substituents independently selected from halogen and C3-C6 cycloalkyl, and wherein 1-2 alkylene units are optionally replaced by O, N, C3-C6 cycloalkyl, or phenyl;
R¹ is azido, tetrazinyl, a C2-C3 alkyne, or an optionally substituted C8-C12 cycloalkyne.

In some embodiments, L¹ is a bond or a C1-C6 alkylene optionally substituted with 1-2 substituents independently selected from halogen and C3-C6 cycloalkyl, and wherein 1-2 alkylene units are optionally replaced by O, N, C3-C6 cycloalkyl, —C(O)—, or phenyl.

Some embodiments provide a protein comprising an antigen-binding domain, wherein the antigen-binding domain comprises a modified phenylalanine residue, the modified phenylalanine residue having the structure:

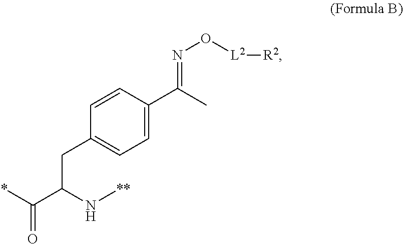

wherein:
* and ** represent the points of connection of the modified phenylalanine residue to the antigen-binding domain;
L² is a C2-C16 alkylene optionally substituted with 1-2 substituents independently selected from halogen and C3-C6 cycloalkyl, and wherein 1-6 alkylene units are optionally replaced by O, N, S, C3-C10 cycloalkyl, phenyl, 5-12 membered heteroaryl, and 4-14 membered heterocyclyl;
R² is a reactive group as described herein, e.g.,

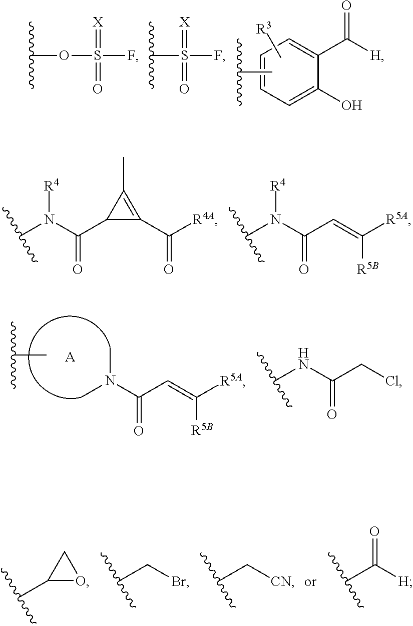

X is O or NR^{X};
R^{X} is hydrogen, C1-C6 alkyl, or C3-C6 cycloalkyl;
R³ is halogen or C1-C6 alkyl;
R⁴ is hydrogen or C1-C6 alkyl;
R^{4A} is C1-C6 alkyl or C3-C6 cycloalkyl;
R^{5A} and R^{5B} are independently hydrogen, halogen, or C1-C6 alkyl; or
R^{5A} and R^{5B} together with the carbon atom to which they are attached form a cyclopropyl; Ring A is a 4-10 membered heterocyclyl.

In some embodiments of Formula (B), L² is a C2-C16 alkylene optionally substituted with 1-2 substituents independently selected from halogen and C3-C6 cycloalkyl, and wherein 1-6 alkylene units are optionally replaced by O, N, S, C3-C10 cycloalkyl, phenyl, 5-12 membered heteroaryl, —C(O)—, and 4-14 membered heterocyclyl; and the other variables defined in Formula (B) are as defined above.

In other embodiments of Formula B, L² is a C1-C16 alkylene optionally substituted with 1-2 substituents independently selected from halogen and C3-C6 cycloalkyl, and wherein 1-6 alkylene units are optionally replaced by O, N, S, C3-C10 cycloalkyl, phenyl, 5-12 membered heteroaryl, —C(O)—, and 4-14 membered heterocyclyl; and the other variables defined in Formula (B) are as defined above

Some embodiments provide a protein comprising an antigen-binding domain, wherein the antigen-binding domain comprises a modified cysteine residue, the modified cysteine residue having the structure:

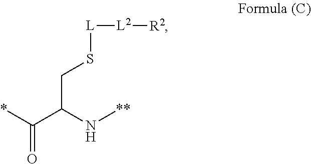

wherein:
* and ** represent the points of connection of the modified cysteine residue to the antigen-binding domain;
L is a bond,

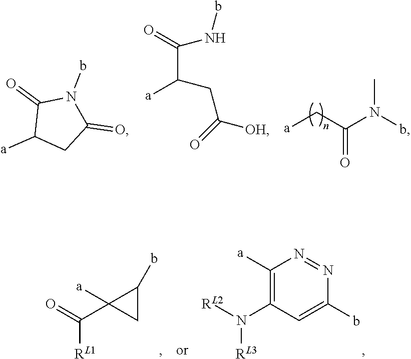

wherein a represents the point of connection of L to the sulfur atom of the modified cysteine residue and “b” represents the point of connection of L to L²;
n is 1 or 2;
R^{L1}, R^{L2}, and R^{L3}, are each independently selected C1-C10 alkyl;
L² is a C2-C16 alkylene optionally substituted with 1-2 substituents independently selected from halogen and C3-C6 cycloalkyl, and wherein 1-6 alkylene units are optionally replaced by O, N, S, C3-C10 cycloalkyl, phenyl, 5-12 membered heteroaryl, and 4-14 membered heterocyclyl;
   R² is a reactive group as described herein, e.g.,

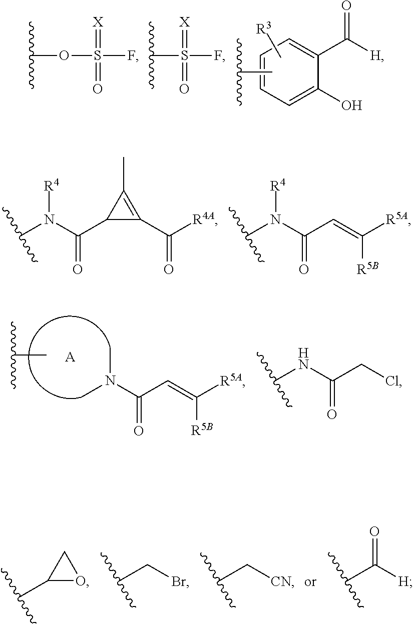

X is O or NR^{X};
R^{X} is hydrogen, C1-C6 alkyl, or C3-C6 cycloalkyl;
R³ is halogen or C1-C6 alkyl;
R⁴ is hydrogen or C1-C6 alkyl;
R^{4A} is C1-C6 alkyl or C3-C6 cycloalkyl;
R^{5A} and R^{5B} are independently hydrogen, halogen, or C1-C6 alkyl; or
R^{5A} and R^{5B} together with the carbon atom to which they are attached form a cyclopropyl;
Ring A is a 4-10 membered heterocyclyl.

In some embodiments of Formula (C), L² is a C2-C16 alkylene optionally substituted with 1-2 substituents independently selected from halogen and C3-C6 cycloalkyl, and wherein 1-6 alkylene units are optionally replaced by O, N, S, C3-C10 cycloalkyl, phenyl, 5-12 membered heteroaryl, —C(O)—, and 4-14 membered heterocyclyl; and the other variables defined in Formula (C) are as defined above.

In some embodiments of Formula (C), L² is a C1-C16 alkylene optionally substituted with 1-2 substituents independently selected from halogen and C3-C6 cycloalkyl, and wherein 1-6 alkylene units are optionally replaced by O, N, S, C3-C10 cycloalkyl, phenyl, 5-12 membered heteroaryl, —C(O)—, and 4-14 membered heterocyclyl; and the other variables defined in Formula (C) are as defined above.

In some embodiments of Formula (C), it is provided that the structure does not contain a GLP-1 peptide or variant thereof, optionally wherein the structure does not contain a modified GLP-1 peptide, optionally wherein the structure does not contain a peptide as described in WO 2025/076010.

In some embodiments, R² can further include —OSO₂—HET as described herein.

Some embodiments provide a pharmaceutical composition comprising a protein described herein and at least one pharmaceutically acceptable excipient.

Some embodiments provide a kit comprising (a) a protein as described herein and (b) a pharmaceutically acceptable excipient.

Some embodiments provide a kit comprising the pharmaceutical composition described herein and instructions for administration of the pharmaceutical composition to a human subject.

Some embodiments provide a method of treating in a subject in need thereof, comprising administering to the subject therapeutically effective amount of a protein described herein, or a pharmaceutical composition described herein.

Some embodiments provide a method of inducing or increasing internalization of the protein into a mammalian cell that expresses the target protein comprising contacting the mammalian cell with a protein described herein.

Some embodiments provide a method of inhibiting the activity of the target protein in a mammalian cell, comprising contacting the target protein with a protein described herein.

Some embodiments provide a method of reducing the amount of the target protein in a mammalian cell comprising the target protein, the method comprising contacting the target protein with a protein described herein.

Some embodiments provide a method of inducing cell death in a mammalian cell comprising the target protein, the method comprising contacting the cell with a protein described herein.

Some embodiments provide a method of screening for a protein that forms a covalent bond with a target protein in a mammalian cell, the method comprising:
contacting the target protein with a protein described herein; and
determining whether a covalent bond has been formed between the protein and the target protein.

Some embodiments provide a protein-protein conjugate comprising a first protein A and a second protein B, wherein the protein-protein conjugate has the structure:

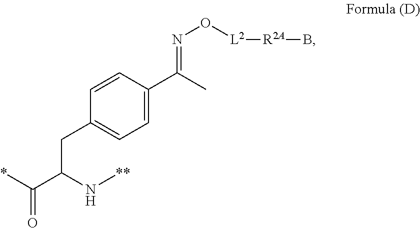

wherein the first protein comprises an antigen-binding domain, wherein the antigen-binding domain comprises a modified phenylalanine residue, wherein:
* and ** represent the points of connection of the modified phenylalanine residue to the antigen-binding domain of the first protein A;
L² is a C2-C16 alkylene optionally substituted with 1-2 substituents independently selected from halogen and C3-C6 cycloalkyl, and wherein 1-6 alkylene units are optionally replaced by O, N, S, C3-C10 cycloalkyl, phenyl, 5-12 membered heteroaryl, and 4-14 membered heterocyclyl;
R^{2A} is

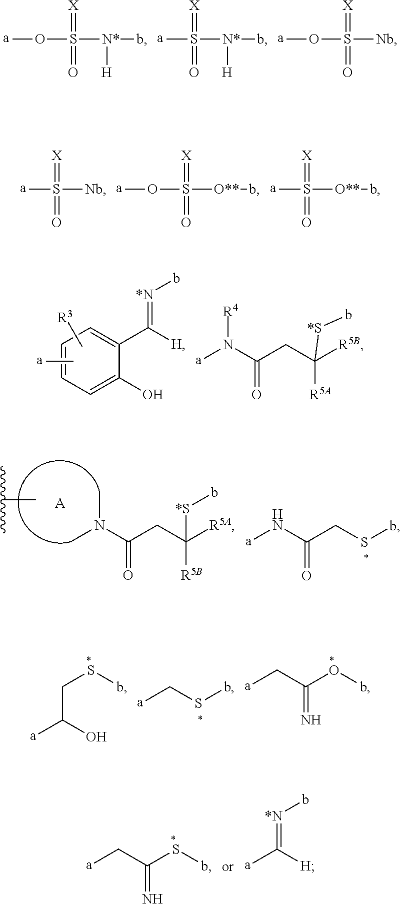

“a” represents the connection of R^{2A} to L², “b” represents the connection of R^{2A} to protein B, N* is a nitrogen atom of a lysine residue of protein B, S* is a sulfur atom of a cysteine residue of protein B, O* is an oxygen atom from a serine residue or a threonine residue of protein B, Nb 15 is the nitrogen atom of a histidine residue of protein B and the connection of R^{2A} to protein B, and O** is an oxygen atom from a tyrosine residue of protein B;
X is O or NR^{X};
R^{X} is hydrogen, C1-C6 alkyl, or C3-C6 cycloalkyl;
R³ is halogen or C1-C6 alkyl;
R⁴ is hydrogen or C1-C6 alkyl;
R^{4A} is C1-C6 alkyl or C3-C6 cycloalkyl;
R^{5A} and R^{5B} are independently hydrogen, halogen, or C1-C6 alkyl; or
R^{5A} and R^{5B} together with the carbon atom to which they are attached form a cyclopropyl;
Ring A is a 4-10 membered heterocyclyl;
wherein the antigen-binding domain of the first protein A specifically binds to the second protein B.

In some embodiments of Formula (D), L² is a C2-C16 alkylene optionally substituted with 1-2 substituents independently selected from halogen and C3-C6 cycloalkyl, and wherein 1-6 alkylene units are optionally replaced by O, N, S, C3-C10 cycloalkyl, phenyl, 5-12 membered heteroaryl, —C(O)—, and 4-14 membered heterocyclyl; and the other variables defined in Formula (D) are as defined above.

In some embodiments of Formula (D), L² is a C1-C16 alkylene optionally substituted with 1-2 substituents independently selected from halogen and C3-C6 cycloalkyl, and wherein 1-6 alkylene units are optionally replaced by O, N, S, C3-C10 cycloalkyl, phenyl, 5-12 membered heteroaryl, —C(O)—, and 4-14 membered heterocyclyl; and the other variables defined in Formula (D) are as defined above.

Some embodiments provide a protein-protein conjugate comprising a first protein A and a second protein B, wherein the protein-protein conjugate has the structure:

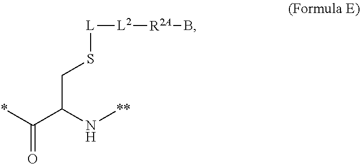

wherein the first protein A comprises an antigen-binding domain, wherein the antigen-binding domain comprises a modified cysteine residue, wherein:
* and ** represent the points of connection of the modified cysteine residue to the antigen-binding domain;
L is a bond,

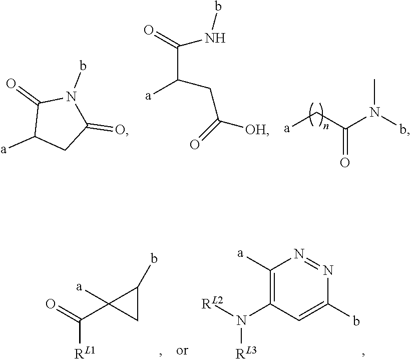

wherein “a” represents the point of connection of L to the sulfur atom of the modified cysteine residue and “b” represents the point of connection of L to L²;
n is 1 or 2;
R^{L1}, R^{L2}, and R^{L3}, are each independently selected C1-C10 alkyl;
L² is a C2-C16 alkylene optionally substituted with 1-2 substituents independently selected from halogen and C3-C6 cycloalkyl, and wherein 1-6 alkylene units are optionally replaced by O, N, S, C3-C10 cycloalkyl, phenyl, 5-12 membered heteroaryl, and 4-14 membered heterocyclyl;
R^{2A} is

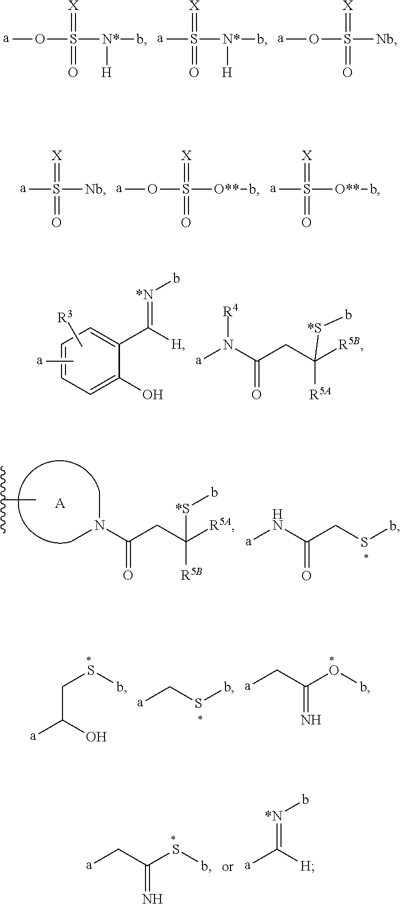

“a” represents the connection of R^{2A} to L², “b” represents the connection of R^{2A} to protein B, N* is a nitrogen atom of a lysine residue of protein B, S* is a sulfur atom of a cysteine residue of protein B, O* is an oxygen atom from a serine residue or a threonine residue of protein B, Nb is the nitrogen atom of a histidine residue of protein B and the connection of R^{2A} to protein B, and O** is an oxygen atom from a tyrosine residue of protein B;
X is O or NR^{X};
R^{X} is hydrogen, C1-C6 alkyl, or C3-C6 cycloalkyl;
R³ is halogen or C1-C6 alkyl;
R⁴ is hydrogen or C1-C6 alkyl;
R^{4A} is C1-C6 alkyl or C3-C6 cycloalkyl;
R^{5A} and R^{5B} are independently hydrogen, halogen, or C1-C6 alkyl; or
R^{5A} and R^{5B} together with the carbon atom to which they are attached form a cyclopropyl;
Ring A is a 4-10 membered heterocyclyl,
wherein the antigen-binding domain of the first protein A specifically binds to the second protein B.

In some embodiments of Formula (E), L² is a C2-C16 alkylene optionally substituted with 1-2 substituents independently selected from halogen and C3-C6 cycloalkyl, and wherein 1-6 alkylene units are optionally replaced by O, N, S, C3-C10 cycloalkyl, phenyl, 5-12 membered heteroaryl, —C(O)—, and 4-14 membered heterocyclyl; and the other variables defined in Formula (E) are as defined above.

In some embodiments of Formula (E), L² is a C1-C16 alkylene optionally substituted with 1-2 substituents independently selected from halogen and C3-C6 cycloalkyl, and wherein 1-6 alkylene units are optionally replaced by O, N, S, C3-C10 cycloalkyl, phenyl, 5-12 membered heteroaryl, —C(O)—, and 4-14 membered heterocyclyl; and the other variables defined in Formula (E) are as defined above.

In some embodiments of Formula (E), it is provided that the structure does not contain a GLP-1 peptide or variant thereof, optionally wherein the structure does not contain a modified GLP-1 peptide, optionally wherein the structure does not contain a peptide as described in WO 2025/076010.

Some embodiments provide a method of making a protein comprising an antigen-binding domain, wherein the antigen-binding domain comprises a modified phenylalanine residue, the modified phenylalanine residue having the structure:

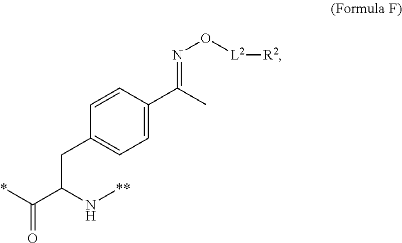

the method comprising contacting
(a) a compound having the structure Z—R² with
(b) a protein comprising an antigen-binding domain, wherein the antigen-binding domain comprising an oxime, the oxime having the structure:

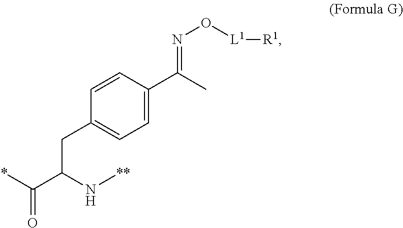

wherein:
Z reacts with -L¹- to form -L²-, wherein when R¹ is azido or tetrazinyl, then Z is a C2-C3 alkyne or an optionally substituted C8-C12 cycloalkyne, and when R¹ is a C2-C3 alkyne or an optionally substituted C8-C12 cycloalkyne, then Z is azido or tetrazinyl;
* and ** represent the points of connection of the oxime to the antigen-binding domain;
L² is a C2-C16 alkylene optionally substituted with 1-2 substituents independently selected from halogen and C3-C6 cycloalkyl, and wherein 1-6 alkylene units are optionally replaced by O, N, S, C3-C10 cycloalkyl, phenyl, 5-12 membered heteroaryl, and 4-14 membered heterocyclyl;
R² is

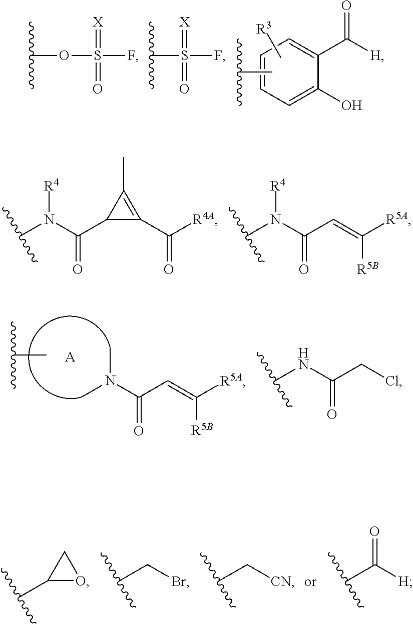

X is O or NR^{X};
R^{X} is hydrogen, C1-C6 alkyl, or C3-C6 cycloalkyl;
R³ is halogen or C1-C6 alkyl;
R⁴ is hydrogen or C1-C6 alkyl;
R^{4A} is C1-C6 alkyl or C3-C6 cycloalkyl;
R^{5A} and R^{5B} are independently hydrogen, halogen, or C1-C6 alkyl; or
R^{5A} and R^{5B} together with the carbon atom to which they are attached form a cyclopropyl;
Ring A is a 4-10 membered heterocyclyl;
L¹ is a bond or a C1-C6 alkylene optionally substituted with 1-2 substituents independently selected from halogen and C3-C6 cycloalkyl, and wherein 1-2 alkylene units are optionally replaced by O, N, C3-C6 cycloalkyl, or phenyl; and
R¹ is azido, tetrazinyl, a C2-C3 alkyne, or an optionally substituted C8-C12 cycloalkyne.

In some embodiments of Formulas (F) and (G), L² is a C2-C16 alkylene optionally substituted with 1-2 substituents independently selected from halogen and C3-C6 cycloalkyl, and wherein 1-6 alkylene units are optionally replaced by O, N, S, C3-C10 cycloalkyl, phenyl, 5-12 membered heteroaryl, —C(O)—, and 4-14 membered heterocyclyl; and the other variables defined in Formulas (F) and (G) are as defined above.

In some embodiments of Formulas (F) and (G), L² is a C1-C16 alkylene optionally substituted with 1-2 substituents independently selected from halogen and C3-C6 cycloalkyl, and wherein 1-6 alkylene units are optionally replaced by O, N, S, C3-C10 cycloalkyl, phenyl, 5-12 membered heteroaryl, —C(O)—, and 4-14 membered heterocyclyl; and the other variables defined in Formulas (F) and (G) are as defined above.

In some of the foregoing embodiments of Formulas (A), (B), (C), (D), €, (F), and (G), R² can further include —OSO₂—HET.

### Additional Definitions

To facilitate understanding of the disclosure set forth herein, a number of additional terms are defined below. Generally, the nomenclature used herein and the laboratory procedures in organic chemistry, medicinal chemistry, and pharmacology described herein are those well-known and commonly employed in the art. Unless defined otherwise, all technical and scientific terms used herein generally have the same meaning as commonly understood by one of ordinary skill in the art to which this disclosure belongs. Each of the patents, applications, published applications, and other publications that are mentioned throughout the specification and the attached appendices are incorporated herein by reference in their entireties.

The term “about” when referring to a number or a numerical range means that the number or numerical range referred to is an approximation, for example, within experimental variability and/or statistical experimental error, and thus the number or numerical range may vary up to ±10% of the stated number or numerical range.

The term “acceptable” with respect to a formulation, composition or ingredient, as used herein, means having no persistent detrimental effect on the general health of the subject being treated.

An “antigen-binding domain” is one or more protein domain(s) (e.g., formed from amino acids from a single polypeptide or formed from amino acids from two or more polypeptides (e.g., the same or different polypeptides) that is capable of specifically binding to one or more different antigen(s). In some examples, an antigen-binding domain can bind to an antigen or epitope with specificity and affinity similar to that of naturally-occurring antibodies. In some embodiments, the antigen-binding domain can be an antibody or a fragment thereof. In some embodiments, an antigen-binding domain can include an alternative scaffold. Non-limiting examples of antigen-binding domains are described herein. Additional examples of antigen-binding domains are known in the art. In some embodiments, an antigen-binding domain can be a ligand for a target receptor protein. In some embodiments, an antigen-binding domain can be a soluble receptor protein. In some embodiments, an antigen-binding domain can be a peptide substrate for an enzyme.

The term “antibody” is used herein in its broadest sense and includes certain types of immunoglobulin molecules that include one or more antigen-binding domains that specifically bind to an antigen or epitope. An antibody specifically includes, e.g., intact antibodies (e.g., intact immunoglobulins, e.g., human IgG (e.g., human IgG1, human IgG2, human IgG3, human IgG4)), antibody fragments, and multi-specific antibodies. One example of an antigen-binding domain is an antigen-binding domain formed by a VH-VL dimer. Additional examples of an antibody are described herein. Additional examples of an antibody are known in the art.

The phrase “cytostatic to a cell” refers to a direct or indirect decrease in the proliferation (cell division) of the cell (e.g., a cancer cell) in vivo or in vitro. When an agent is cytostatic to a cell, the agent can, e.g., directly or indirectly result in cell cycle arrest of the cell (e.g., a cancer cell). In some examples, an agent that is cytostatic to a cell can reduce the number of cells in a population of the cells that are in S phase (as compared to the number of cells in a population of the cells that are in S phase prior to contact with the agent). In some examples, an agent that is cytostatic to a cell can reduce the percentage of the cells in S phase by at least 20%, at least 40%, at least 60%, or at least 80% (e.g., as compared to the percentage of cells in a population of the cells that are in S phase prior to contact with the agent).

The phrase “cytotoxic to a cell” refers to the inducement, directly or indirectly, in the death (e.g., necrosis or apoptosis) of the cell (e.g., a mammalian cell, e.g., a cancer cell).

The term “pharmaceutically acceptable excipient” means a pharmaceutically-acceptable material, composition, or vehicle, such as a liquid or solid filler, diluent, carrier, solvent, or encapsulating material. In one embodiment, each component is “pharmaceutically acceptable” in the sense of being compatible with the other ingredients of a pharmaceutical formulation, and suitable for use in contact with the tissue or organ of humans and animals without excessive toxicity, irritation, allergic response, immunogenicity, or other problems or complications, commensurate with a reasonable benefit/risk ratio. See, e.g., Remington: The Science andPractice of Pharmacy, 21st ed.; Lippincott Williams & Wilkins: Philadelphia, PA, 2005; Handbook of Pharmaceutical Excipients, 6th ed.; Rowe et al., Eds.; The Pharmaceutical Press and the American Pharmaceutical Association: 2009; Handbook of Pharmaceutical Additives, 3rd ed.; Ash and Ash Eds.; Gower Publishing Company: 2007; Pharmaceutical Preformulation and Formulation, 2nd ed.; Gibson Ed.; CRC Press LLC: Boca Raton, FL, 2009.

As used herein, the “subject” refers to any animal, including mammals such as primates (e.g., humans), mice, rats, other rodents, rabbits, dogs, cats, swine, cattle, sheep, horses, primates, and humans. In some embodiments, the subject is a human. In some embodiments, the subject has experienced and/or exhibited at least one symptom of the disease to be treated.

As used herein a “therapeutically effective amount” means an amount of an entity (e.g, an ADC as described herein) that, when administered to a subject in need of such treatment, is sufficient to (i) treat a particular disease, (ii) attenuate, ameliorate, or eliminate one or more symptoms of the particular disease, or (iii) delay the onset of one or more symptoms of the particular disease, as described herein.

As used herein, terms “treat” or “treatment” refer to therapeutic or palliative measures. Beneficial or desired clinical results include, but are not limited to, alleviation, in whole or in part, of symptoms associated with a disease, diminishment of the extent of the disease, stabilized (i.e., not worsening) state of disease, delay or slowing of cancer progression, amelioration or palliation of the disease state (e.g., one or more symptoms of the disease), and remission (whether partial or total), whether detectable or undetectable. “Treatment” can also mean prolonging survival as compared to expected survival if not receiving treatment.

Whenever a group is described as being “optionally substituted” that group may be unsubstituted or substituted with one or more of the indicated substituents. Likewise, when a group is described as being “substituted” the substituent(s) may be selected from one or more the indicated substituents. If no substituents are indicated, it is meant that the indicated “optionally substituted” or “substituted” group may be substituted with one or more individually and independently selected group(s) that are stable and chemically acceptable for the group being substituted. Non-limiting examples of optional substituents are halogen, cyano, hydroxyl, nitro, nitroso, azido, sulfhydryl, acyl, alkyl, hydroxyalkyl, aminoalkyl, alkoxyamino, haloalkyl, alkenyl, haloalkenyl, alkynyl, haloalkynyl, alkoxy, hydroxyalkoxy, alkoxyalkoxy, alkenoxy, alkynoxy, haloalkoxy, haloalkenoxy, haloalkynoxy, cycloalkyl, halocycloalkyl, cycloalkoxy, aryl, aryloxy, heteroaryl, heteroaryloxy, heterocyclyl, heterocyclyloxy, aralkyl, cycloalkylalkyl, heteroaralkyl, alkoxyalkyl, heterocyclylalkyl, thiocarbonyl, 0-carbamyl, N-carbamyl, O-thiocarbamyl, N-thiocarbamyl, alkoxycarbonyl, C-amido, N-amido, S-sulfonamido, N-sulfonamido, sulfenyl, halosulfenyl, sulfonyl, sulfinyl, sulfoximino, sulfonimidamido, phosphine oxide, C-carboxy, O-carboxy, arylalkoxy, cycloalkylalkoxy, carboxaldehyde, iminyl, trihalomethanesulfonyl, trihalomethanesulfonamido, and ureido.

The term “halogen” refers to fluoro (F), chloro (Cl), bromo (Br), or iodo (I).

The term “oxo” refers to a divalent doubly bonded oxygen atom (i.e., “═O”). As used herein, oxo groups are attached to carbon atoms to form carbonyls.

The term “hydroxyl” refers to an —OH radical.

The term “sulfhydryl” refers to a —SH radical.

The term “cyano” refers to a —CN radical.

The term “azido” refers to a —N₃ radical.

The term “nitro” refers to a —NO₂ radical.

The term “nitroso” refers to a —N═O radical.

The term “alkyl” refers to a saturated acyclic hydrocarbon radical that may be a straight chain or branched chain, containing the indicated number of carbon atoms. For example, C1-C10 indicates that the group may have from 1 to 10 (inclusive) carbon atoms in it. Non-limiting examples include methyl, ethyl, iso-propyl, tert-butyl, n-hexyl. The term “saturated” as used in this context means only single bonds present between constituent carbon atoms and other available valences occupied by hydrogen and/or other substituents as defined herein.

The term “alkylene” refers to a bivalent alkyl group, as described herein, that may be a straight chain or branched chain, containing the indicated number of carbon atoms. Non-limiting examples include methylene, ethylene, 1-propylene, 2-propylene, 2-methyl-2-propylene, tert-butylene, n-hexylene. An alkylene can optionally include one or more oxo (C═O) groups.

When an alkylene group, as described herein, is substituted with, interrupted by, and/or has a methylene group “replaced” by a particular substituent or heteroatom, the substituent can be connected in any appropriate way (e.g., valence, stability, and the like). For example, it is to be understood that an alkylene having two methylene groups replaced by oxygen atoms refers to two non-adjacent methylene groups (e.g., the oxygen atoms do not form a peroxide). In addition, the substitution of a cyclopropyl group on an alkylene refers to, for example,

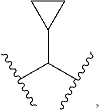 [SMILES: *C(*)C1CC1]

while the replacement of a methylene of an alkylene refers to, for example,

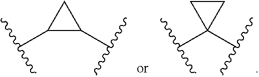 [SMILES: *C1CC1* ; *C1(*)CC1]

As used herein, “alkenyl” refers to an alkyl group that contains in the straight or branched hydrocarbon chain one or more double bonds.

As used herein, “alkynyl” refers to an alkyl group that contains in the straight or branched hydrocarbon chain one or more triple bonds.

The term “aryl” refers to a 6-20 carbon mono-, bi-, tri- or polycyclic group wherein at least one ring in the system is aromatic (e.g., 6-carbon monocyclic, 10-carbon bicyclic, or 14-carbon tricyclic aromatic ring system); and wherein 0, 1, 2, 3, or 4 atoms of each ring may be substituted by a substituent. Examples of aryl groups include phenyl, naphthyl, tetrahydronaphthyl, and the like.

The term “cycloalkyl” as used herein refers to cyclic saturated or partially unsaturated hydrocarbon groups having, e.g., 3 to 20 ring carbons, preferably 3 to 16 ring carbons, and more preferably 3 to 12 ring carbons or 3-10 ring carbons or 3-6 ring carbons, wherein the cycloalkyl group may be optionally substituted. Examples of cycloalkyl groups include, without limitation, cyclopropyl, cyclobutyl, cyclopentyl, cyclopentenyl, cyclohexyl, cyclohexenyl, cycloheptyl, and cyclooctyl. Cycloalkyl may include multiple fused and/or bridged rings. Non-limiting examples of fused/bridged cycloalkyl includes: bicyclo[1.1.0]butane, bicyclo[2.1.0]pentane, bicyclo[1.1.1]pentane, bicyclo[3.1.0]hexane, bicyclo[2.1.1]hexane, bicyclo[3.2.0]heptane, bicyclo[4.1.0]heptane, bicyclo[2.2.1]heptane, bicyclo[3.1.1]heptane, bicyclo[4.2.0]octane, bicyclo[3.2.1]octane, bicyclo[2.2.2]octane, and the like. Cycloalkyl also includes spirocyclic rings (e.g., spirocyclic bicycle wherein two rings are connected through just one atom). Non-limiting examples of spirocyclic cycloalkyls include spiro[2.2]pentane, spiro[2.5]octane, spiro[3.5]nonane, spiro[3.5]nonane, spiro[3.5]nonane, spiro[4.4]nonane, spiro[2.6]nonane, spiro[4.5]decane, spiro[3.6]decane, spiro[5.5]undecane, and the like. The term “saturated” as used in this context means only single bonds present between constituent carbon atoms.

The term “heteroaryl”, as used herein, means a mono-, bi-, tri- or polycyclic group having 5 to 20 ring atoms, alternatively 5, 6, 9, 10, or 14 ring atoms; wherein at least one ring in the system contains one or more heteroatoms independently selected from the group consisting of N, O, S, P, B, and Si and at least one ring in the system is aromatic (but does not have to be a ring which contains a heteroatom, e.g. tetrahydroisoquinolinyl, e.g., tetrahydroquinolinyl). Examples of heteroaryl include thienyl, pyridinyl, furyl, oxazolyl, oxadiazolyl, pyrrolyl, imidazolyl, triazolyl, thiodiazolyl, pyrazolyl, isoxazolyl, thiadiazolyl, pyranyl, pyrazinyl, pyrimidinyl, pyridazinyl, triazinyl, thiazolyl benzothienyl, benzoxadiazolyl, benzofuranyl, benzimidazolyl, benzotriazolyl, cinnolinyl, indazolyl, indolyl, isoquinolinyl, isothiazolyl, naphthyridinyl, purinyl, thienopyridinyl, pyrido[2,3-d]pyrimidinyl, pyrrolo[2,3-b]pyridinyl, quinazolinyl, quinolinyl, thieno[2,3-c]pyridinyl, pyrazolo[3,4-b]pyridinyl, pyrazolo[3,4-c]pyridinyl, pyrazolo[4,3-c]pyridine, pyrazolo[4,3-b]pyridinyl, tetrazolyl, chromane, 2,3-dihydrobenzo[b][1,4]dioxine, benzo[d][1,3]dioxole, 2,3-dihydrobenzofuran, tetrahydroquinoline, 2,3-dihydrobenzo[b][1,4]oxathiine, isoindoline, and others. In some embodiments, the heteroaryl is selected from thienyl, pyridinyl, furyl, pyrazolyl, imidazolyl, isoindolinyl, pyranyl, pyrazinyl, and pyrimidinyl. For purposes of clarification, heteroaryl also includes aromatic lactams, aromatic cyclic ureas, or vinylogous analogs thereof, in which each ring nitrogen adjacent to a carbonyl is tertiary (i.e., all three valences are occupied by non-hydrogen substituents), such as one or more of pyridone

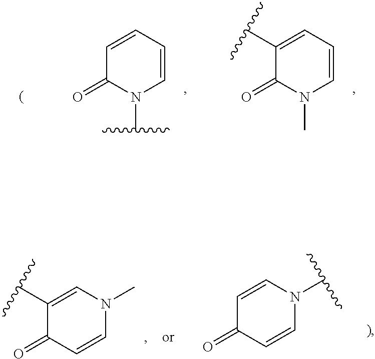

pyrimidone

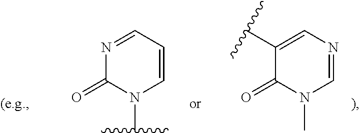

pyridazinone

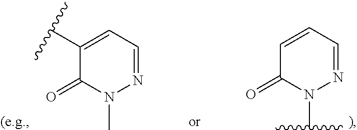

pyrazinone

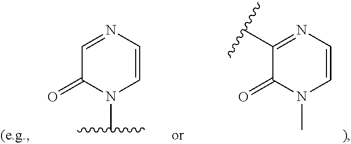

and imidazolone

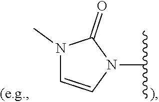 [SMILES: *n1ccn(C)c1=O]

wherein each ring nitrogen adjacent to a carbonyl is tertiary (i.e., the oxo group (i.e., “═O”) herein is a constituent part of the heteroaryl ring).

The term “heterocyclyl” refers to a mono-, bi-, tri-, or polycyclic saturated or partially unsaturated ring system with 3-16 ring atoms (e.g., 5-8 membered monocyclic, 8-12 membered bicyclic, or 11-14 membered tricyclic ring system) having 1-3 heteroatoms if monocyclic, 1-6 heteroatoms if bicyclic, or 1-9 heteroatoms if tricyclic or polycyclic, said heteroatoms selected from O, N, P, S, B, or Si (e.g., carbon atoms and 1-3, 1-6, or 1-9 heteroatoms of N, O, P, S, B, or Si if monocyclic, bicyclic, or tricyclic, respectively), wherein one or more ring atoms may be substituted by 1-3 oxo (forming, e.g., a lactam or phosphinane oxide) and one or more N or S atoms may be substituted by 1-2 oxido (forming, e.g., an N-oxide, an S-oxide, or an S,S-dioxide), valence permitting; and wherein 0, 1, 2 or 3 atoms of each ring may be substituted by 1-2 substituents. Examples of heterocyclyl groups include piperazinyl, pyrrolidinyl, dioxanyl, morpholinyl, tetrahydrofuranyl, tetrahydropyridyl, dihydropyrazinyl, dihydropyridyl, dihydropyrrolyl, dihydrofuranyl, dihydrothiophenyl, oxaphosphinanyl oxide, azaphosphinanyl oxide, and the like. Heterocyclyl may include multiple fused and bridged rings. Non-limiting examples of fused/bridged heteorocyclyl includes: 2-azabicyclo[1.1.0]butane, 2-azabicyclo[2.1.0]pentane, 2-azabicyclo[1.1.1]pentane, 3-azabicyclo[3.1.0]hexane, 5-azabicyclo[2.1.1]hexane, 3-azabicyclo[3.2.0]heptane, octahydrocyclopenta[c]pyrrole, 3-azabicyclo[4.1.0]heptane, 7-azabicyclo[2.2.1]heptane, 6-azabicyclo[3.1.1]heptane, 7-azabicyclo[4.2.0]octane, 2-azabicyclo[2.2.2]octane, 3-azabicyclo[3.2.1]octane, 2-oxabicyclo[1.1.0]butane, 2-oxabicyclo[2.1.0]pentane, 2-oxabicyclo[1.1.1]pentane, 3-oxabicyclo[3.1.0]hexane, 5-oxabicyclo[2.1.1]hexane, 3-oxabicyclo[3.2.0]heptane, 3-oxabicyclo[4.1.0]heptane, 7-oxabicyclo[2.2.1]heptane, 6-oxabicyclo[3.1.1]heptane, 7-oxabicyclo[4.2.0]octane, 2-oxabicyclo[2.2.2]octane, 3-oxabicyclo[3.2.1]octane, and the like. Heterocyclyl also includes spirocyclic rings (e.g., spirocyclic bicycle wherein two rings are connected through just one atom). Non-limiting examples of spirocyclic heterocyclyls include 2-azaspiro[2.2]pentane, 4-azaspiro[2.5]octane, 1-azaspiro[3.5]nonane, 2-azaspiro[3.5]nonane, 7-azaspiro[3.5]nonane, 2-azaspiro[4.4]nonane, 6-azaspiro[2.6]nonane, 1,7-diazaspiro[4.5]decane, 7-azaspiro[4.5]decane 2,5-diazaspiro[3.6]decane, 3-azaspiro[5.5]undecane, 2-oxaspiro[2.2]pentane, 4-oxaspiro[2.5]octane, 1-oxaspiro[3.5]nonane, 2-oxaspiro[3.5]nonane, 7-oxaspiro[3.5]nonane, 2-oxaspiro[4.4]nonane, 6-oxaspiro[2.6]nonane, 1,7-dioxaspiro[4.5]decane, 2,5-dioxaspiro[3.6]decane, 1-oxaspiro[5.5]undecane, 3-oxaspiro[5.5]undecane, 3-oxa-9-azaspiro[5.5]undecane and the like.

As used herein, examples of aromatic rings include: benzene, pyridine, pyrimidine, pyrazine, pyridazine, pyridone, pyrrole, pyrazole, oxazole, thioazole, isoxazole, isothiazole, and the like.

The term “haloalkyl” refers to an alkyl, in which one or more hydrogen atoms is/are replaced with an independently selected halogen.

The term “halocycloalkyl” refers to a cycloalkyl, in which one or more hydrogen atoms is/are replaced with an independently selected halogen.

The term “hydroxyalkyl” refers to an alkyl, in which one or more hydrogen atoms is/are replaced with hydroxyl.

The term “haloalkenyl” refers to an alkenyl, in which one or more hydrogen atoms is/are replaced with an independently selected halogen.

The term “haloalkynyl” refers to an alkynyl, in which one or more hydrogen atoms is/are replaced with an independently selected halogen.

The term “alkoxy” refers to an —O-alkyl radical (e.g., —OCH₃).

The term “alkoxyalkyl” refers to an alkyl, in which one or two hydrogen atoms is/are replaced with an independently selected alkoxy (e.g., methoxyethyl).

The term “hydroxyalkoxy” refers to an alkoxy group, in which one or two hydrogen atoms is/are replaced with hydroxy.

The term “alkoxyalkoxy” refers to an alkoxy group, in which one or two hydrogen atoms is/are replaced with an independently selected alkoxy.

The term “alkoxyamino” refers to an —O-amino radical (e.g., —OCH₂CH₂N(CH₃)₂).

The term “haloalkoxy” refers to an —O-haloalkyl radical (e.g., —OCF₃).

The term “alkenoxy” refers to an —O-alkenyl radical (e.g., —O-allyl).

The term “haloalkenoxy” refers to an —O-haloalkenyl radical.

The term “alkynoxy” refers to an —O-alkynyl radical (e.g., —O-propargyl).

The term “haloalkynoxy” refers to an —O-haloalkynyl radical.

The term “cycloalkoxy” refers to an —O-cycloalkyl radical (e.g., —O-cyclopropyl).

The term “aryloxy” refers to an —O-aryl radical (e.g., phenoxy).

The term “heteroaryloxy” refers to an —O-heteroaryl radical (e.g., pyridinoxy).

The term “heterocyclyloxy” refers to an —O-heterocyclyl radical (e.g., —O-pyrrolidinyl or —O-oxetanyl).

The term “aralkyl” refer to an aryl group connected, as a substituent, via an alkyl group (e.g., benzyl).

The term “cycloalkylalkyl” refers to a cycloalkyl group connected, as a substituent, via an alkyl group (e.g., ethylcyclobutyl).

The term “heteroaralkyl” refers to a heteroaryl group connected, as a substituent, via an alkyl group (e.g., methylpyrimidinyl).

The term “heterocyclylalkyl” refers to a heterocyclyl group connected, as a substituent, via an alkyl group (e.g., methyloxetanyl).

The term “aralkoxy” refers to an aryl group connected, as a substituent, via an alkoxy group (e.g., benzyloxy).

The term “cycloalkylalkoxy” refers to a cycloalkyl connected, as a substituent, via an alkoxy group (e.g., methoxycyclopropyl).

The term “aminoalkyl” refers to an amino group connected, as a substituent, via an alkyl group (e.g., methyl(dimethylamino)).

A “sulfenyl” group refers to an —SR group in which R can be hydrogen, alkyl, alkenyl, alkynyl, cycloalkyl, cycloalkenyl, aryl, heteroaryl, heterocyclyl, aralkyl heteroaralkyl, heterocyclylalkyl, or cycloalkylalkyl.

A “halosulfenyl” group refers to a sulfenyl, in which one or more hydrogen atoms is/are replaced with an independently selected halogen (e.g., —S(CF₃) or —S(CHF₂)).

A “sulfinyl” group refers to an —S(═O)R group in which R can be the same as defined with respect to sulfenyl.

A “sulfonyl” group refers to an —SO₂R group in which R can be the same as defined with respect to sulfenyl.

A “sulfoximine” group refers to an —S(═O)(=NR)R′, where R is hydrogen, alkyl, haloalkyl, hydroxyalkyl, aminoalkyl, alkenyl, alkynyl, cycloalkyl, cycloalkenyl, aryl, heteroaryl, heterocyclyl, aralkyl heteroaralkyl, heterocyclylalkyl, or cycloalkylalkyl; and where R′ alkyl, haloalkyl, hydroxyalkyl, aminoalkyl, alkenyl, alkynyl, cycloalkyl, cycloalkenyl, aryl, heteroaryl, heterocyclyl, aralkyl heteroaralkyl, heterocyclylalkyl, or cycloalkylalkyl.

A “sulfonimidamido” group refers to an —S(═O)(=NR)NR′R″ where R, R′, and R″ are independently hydrogen, alkyl, haloalkyl, hydroxyalkyl, aminoalkyl, alkenyl, alkynyl, cycloalkyl, cycloalkenyl, aryl, heteroaryl, heterocyclyl, aralkyl heteroaralkyl, heterocyclylalkyl, or cycloalkylalkyl; and where R′ alkyl, haloalkyl, hydroxyalkyl, aminoalkyl, alkenyl, alkynyl, cycloalkyl, cycloalkenyl, aryl, heteroaryl, heterocyclyl, aralkyl heteroaralkyl, heterocyclylalkyl, or cycloalkylalkyl.

An “O-carboxy” group refers to a RC(═O)O— group in which R can be hydrogen, alkyl, alkoxy, hydroxyalkyl, aminoalkyl, alkenyl, alkynyl, cycloalkyl, cycloalkenyl, aryl, heteroaryl, heterocyclyl, aralkyl heteroaralkyl, heterocyclylalkyl, or cycloalkylalkyl.

The terms “ester” and “C-carboxy” refer to a —C(═O)OR group in which R can be the same as defined with respect to O-carboxy.

A “thiocarbonyl” group refers to a —C(═S)R group in which R can be the same as defined with respect to O-carboxy.

A “trihalomethanesulfonyl” group refers to an X₃CSO₂— group wherein each X is a halogen.

A “trihalomethanesulfonamido” group refers to an X₃CS(O)₂N(R′)— group wherein each X is a halogen, and R′ is hydrogen, alkyl, alkenyl, alkynyl, cycloalkyl, cycloalkenyl, aryl, heteroaryl, heterocyclyl, aralkyl heteroaralkyl, heterocyclylalkyl, or cycloalkylalkyl.

An “S-sulfonamido” group refers to a —SO₂N(RR′) group in which R and R′ are independently hydrogen, alkyl, alkoxy, hydroxyalkyl, aminoalkyl, alkenyl, alkynyl, cycloalkyl, cycloalkenyl, aryl, heteroaryl, heterocyclyl, aralkyl heteroaralkyl, heterocyclylalkyl, or cycloalkylalkyl.

An “N-sulfonamido” group refers to a RSO₂N(R′)— group in which R and R′ are independently hydrogen, alkyl, alkoxy, hydroxyalkyl, aminoalkyl, alkenyl, alkynyl, cycloalkyl, cycloalkenyl, aryl, heteroaryl, heterocyclyl, aralkyl heteroaralkyl, heterocyclylalkyl, or cycloalkylalkyl.

An “O-carbamyl” group refers to a —OC(═O)N(RR′) group in which R and R′ are independently hydrogen, alkyl, alkoxy, hydroxyalkyl, aminoalkyl, alkenyl, alkynyl, cycloalkyl, cycloalkenyl, aryl, heteroaryl, heterocyclyl, aralkyl heteroaralkyl, heterocyclylalkyl, or cycloalkylalkyl.

An “N-carbamyl” group refers to an ROC(═O)N(R′)— group in which R and R′ are independently hydrogen, alkyl, alkoxy, hydroxyalkyl, aminoalkyl, alkenyl, alkynyl, cycloalkyl, cycloalkenyl, aryl, heteroaryl, heterocyclyl, aralkyl heteroaralkyl, heterocyclylalkyl, or cycloalkylalkyl.

An “O-thiocarbamyl” group refers to a —OC(═S)N(RR′) group in which R and R′ are independently hydrogen, alkyl, alkoxy, hydroxyalkyl, aminoalkyl, alkenyl, alkynyl, cycloalkyl, cycloalkenyl, aryl, heteroaryl, heterocyclyl, aralkyl heteroaralkyl, heterocyclylalkyl, or cycloalkylalkyl.

An “N-thiocarbamyl” group refers to an ROC(═S)N(R′)— group in which R and R′ are independently hydrogen, alkyl, alkoxy, hydroxyalkyl, aminoalkyl, alkenyl, alkynyl, cycloalkyl, cycloalkenyl, aryl, heteroaryl, heterocyclyl, aralkyl heteroaralkyl, heterocyclylalkyl, or cycloalkylalkyl.

A “C-amido” group refers to a —C(═O)N(RR′) group in which R and R′ are independently hydrogen, alkyl, alkoxy, alkoxyalkyl, hydroxyalkyl, aminoalkyl, alkenyl, alkynyl, cycloalkyl, cycloalkenyl, aryl, heteroaryl, heterocyclyl, aralkyl heteroaralkyl, heterocyclylalkyl, or cycloalkylalkyl.

An “N-amido” group refers to a RC(═O)N(R′) group in which R and R′ are independently hydrogen, alkyl, alkoxy, hydroxyalkyl, aminoalkyl, alkenyl, alkynyl, cycloalkyl, cycloalkenyl, aryl, heteroaryl, heterocyclyl, aralkyl heteroaralkyl, heterocyclylalkyl, or cycloalkylalkyl.

The terms “ureido” or “urea” refer to an —NR(C═O)NR′R″ group, in which R, R′, and R″ are independently hydrogen, hydroxyl, alkyl, haloalkyl, hydroxyalkyl, aminoalkyl, alkenyl, alkynyl, cycloalkyl, cycloalkenyl, aryl, heteroaryl, heterocyclyl, aralkyl heteroaralkyl, heterocyclylalkyl, or cycloalkylalkyl.

The term “carboxaldehyde” refers to a —C(═O)H radical.

The term “imine” or “imino” refers to a —N=R radical, in which R is hydrogen, hydroxyl, alkyl, haloalkyl, alkenyl, alkynyl, cycloalkyl, cycloalkenyl, aryl, heteroaryl, heterocyclyl, aralkyl heteroaralkyl, heterocyclylalkyl, or cycloalkylalkyl.

The term “amino” refers to a —NRR′ radical, where R and R′ are independently hydrogen, alkyl, haloalkyl, hydroxyalkyl, alkenyl, alkynyl, cycloalkyl, cycloalkenyl, aryl, heteroaryl, heterocyclyl, aralkyl heteroaralkyl, heterocyclylalkyl, or cycloalkylalkyl. In some instances, an amino group is —NH₂, a mono-alkyl amine (R is hydrogen and R′ is alkyl) or a dialkylamine (R and R′ are independently selected alkyl).

The term “phosphine oxide” refers to a —P(═O)RR′ radical, where R and R′ are independently alkyl, haloalkyl, hydroxyalkyl, aminoalkyl, alkenyl, alkynyl, cycloalkyl, cycloalkenyl, aryl, heteroaryl, heterocyclyl, aralkyl heteroaralkyl, heterocyclylalkyl, or cycloalkylalkyl. As used herein, when a ring is described as being “partially unsaturated”, it means said ring has one or more additional degrees of unsaturation (in addition to the degree of unsaturation attributed to the ring itself, e.g., one or more double or triple bonds between constituent ring atoms), provided that the ring is not aromatic. Examples of such rings include: cyclopentene, cyclohexene, cycloheptene, dihydropyridine, tetrahydropyridine, dihydropyrrole, dihydrofuran, dihydrothiophene, and the like.

For the avoidance of doubt, and unless otherwise specified, for rings and cyclic groups (e.g., aryl, heteroaryl, heterocyclyl, cycloalkyl, and the like described herein) containing a sufficient number of ring atoms to form bicyclic or higher order ring systems (e.g., tricyclic, polycyclic ring systems), it is understood that such rings and cyclic groups encompass those having fused rings, including those in which the points of fusion are located (i) on adjacent ring atoms (e.g., [x.x.0] ring systems, in which 0 represents a zero atom bridge

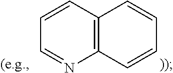

(ii) a single ring atom (spiro-fused ring systems)

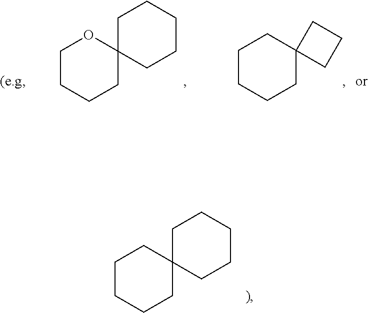 [SMILES: C1CCC2(CC1)CCCCO2 ; C1CCC2(CC1)CCC2 ; C1CCC2(CC1)CCCCC2]

or (iii) a contiguous array of ring atoms (bridged ring systems having all bridge lengths >0)

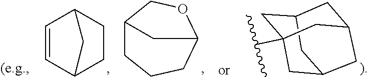

In addition, atoms making up the compounds of the present embodiments are intended to include all isotopic forms of such atoms. Isotopes, as used herein, include those atoms having the same atomic number but different mass numbers. By way of general example and without limitation, isotopes of hydrogen include tritium and deuterium, and isotopes of carbon include ¹³C and ¹⁴C.

In addition, the compounds generically or specifically disclosed herein are intended to include all tautomeric forms. Thus, by way of example, a compound containing the moiety:

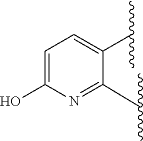 [SMILES: *c1ccc(O)nc1*]

encompasses the tautomeric form containing the moiety:

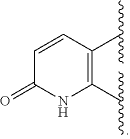 [SMILES: *c1ccc(=O)[nH]c1*]

Similarly, a pyridinyl or pyrimidinyl moiety that is described to be optionally substituted with hydroxyl encompasses pyridone or pyrimidone tautomeric forms.

The compounds provided herein may encompass various stereochemical forms. The compounds also encompass enantiomers (e.g., R and S isomers), diastereomers, as well as mixtures of enantiomers (e.g., R and S isomers) including racemic mixtures and mixtures of diastereomers, as well as individual enantiomers and diastereomers, which arise as a consequence of structural asymmetry in certain compounds. Unless otherwise indicated, when a disclosed compound is named or depicted by a structure without specifying the stereochemistry (e.g., a “flat” structure) and has one or more chiral centers, it is understood to represent all possible stereoisomers of the compound. Likewise, unless otherwise indicated, when a disclosed compound is named or depicted by a structure that specifies the stereochemistry (e.g., a structure with “wedge” and/or “dashed” bonds) and has one or more chiral centers, it is understood to represent the indicated stereoisomer of the compound.

The details of one or more embodiments of this disclosure are set forth in the accompanying drawings and the description below. Other features and advantages of the present disclosure will be apparent from the description and drawings, and from the claims.

### BRIEF DESCRIPTION OF THE DRAWINGS

The following drawings illustrate certain embodiments of the features and advantages of this disclosure. These embodiments are not intended to limit the scope of the appended claims in any manner. Like reference symbols in the drawings indicate like elements.

FIG. 1 shows a crosslinking and gel shift protocol.

FIG. 2 shows a gel shift assay after incubation between the antigen-binding domains shown and EGFR.

FIG. 3 is a gel shift assay showing crosslinking between the antigen-binding domains shown and EGFR after incubation.

FIG. 4 is a gel shift assay showing crosslinking between the antigen-binding domains shown and EGFR with increased run time.

FIG. 5 is a Western blot showing crosslinking between the antigen-binding domains shown and EGFR.

FIG. 6 is a Western blot showing crosslinking between the antigen-binding domains shown and EGFR.

FIG. 7 are graphs (left) showing crosslinking between the antigen-binding domains shown and EGFR over time and a Western blot showing crosslinking between the antigen-binding domain and EGFR.

FIG. 8 shows a crosslinking and gel shift protocol (left) and a gel shift assay after incubation between the antigen-binding domains shown and EGFR.

FIG. 9 is a gel shift assay showing crosslinking between the antigen-binding domains shown and EGFR after incubation.

FIG. 10 are Western blots showing crosslinking between the antigen-binding domains shown and EGFR.

FIG. 11 are Western blots showing crosslinking between the antigen-binding domains shown and EGFR.

FIG. 12 are Western blots showing crosslinking between the antigen-binding domains and EGFR.

FIG. 13 is a crosslinking and gel shift protocol (left) and a gel shift assay (right) after incubation between S31C-MFS-5-1 and EGFR over time.

FIG. 14 is a gel shift assay (left) and graph (right) showing the percent covalency over time.

FIG. 15 are mass spectrometry graphs showing a molecular weight difference between conjugated and non-conjugated antigen-binding domains.

FIG. 16 are Western blots showing covalent conjugation between the antigen-binding domains shown and EGFR in either EGFR+ or EGFR− cell lines.

FIG. 17 are Western blots showing covalent conjugation between the antigen-binding domains shown and EGFR over time.

FIG. 18 shows a crosslinking and gel shift protocol (left) and a gel shift assay (right) after incubation between the antigen-binding domains shown and EGFR.

FIG. 19 shows a crosslinking and gel shift protocol (left) and a gel shift assay (right) after incubation between the antigen-binding domains shown and EGFR.

FIG. 20 shows a crosslinking and gel shift protocol (left) and a gel shift assay (right) after incubation between the antigen-binding domains shown and EGFR.

FIG. 21 shows a crosslinking and gel shift protocol (left) and a gel shift assay (right) after incubation between the antigen-binding domains shown and EGFR.

FIG. 22 shows a crosslinking and gel shift protocol (left) and a gel shift assay (right) after incubation between the antigen-binding domains shown and EGFR.

FIG. 23 shows a graph of the percent covalency of the antigen-binding domains shown and EGFR.

FIG. 24 are Western blots showing crosslinking between the antigen-binding domains and EGFR. Lane values summarized in the table below.

| Lane Number | Value |
|---|---|
| 1 | Ladder |
| 2 | 7D12[S31C] |
| 3 | S31C-MFS-5-1 |
| 4 | S31C-MFS-9-1 |
| 5 | S31C-AU-5 |
| 6 | Y109C-MFS-4-1 |
| 7 | Y109C-Au-2 |
| 8 | None |
| 9 | Ladder |
| 10 | Ladder |
| 11 | 7D12[S31C] |
| 12 | S31C-MFS-5-1 |
| 13 | S31C-MFS-9-1 |
| 14 | S31C-AU-5 |
| 15 | Y109C-MFS-4-1 |
| 16 | Y109C-Au-2 |
| 17 | None |
| 19 | Ladder |

FIG. 25 shows a crosslinking and gel shift protocol (left) and a gel shift assay (right) after incubation between the antigen-binding domains shown and EGFR.

FIG. 26 shows a crosslinking and gel shift protocol (left) and a gel shift assay (right) after incubation between the antigen-binding domains shown and EGFR.

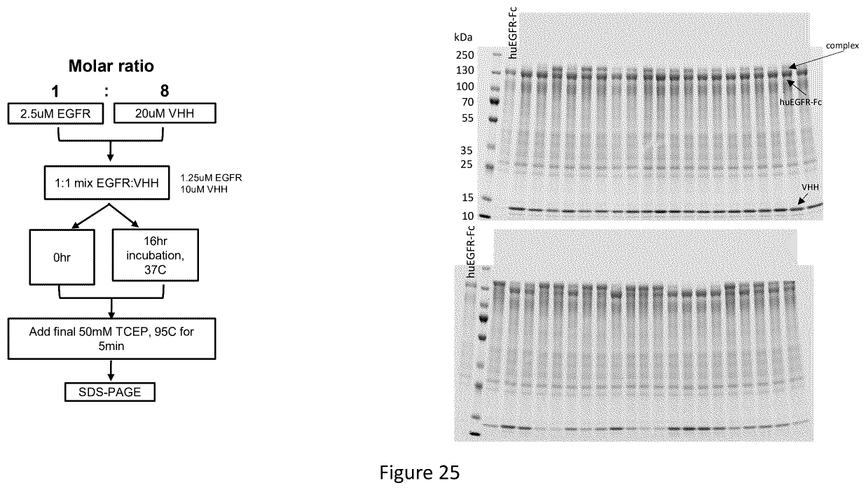

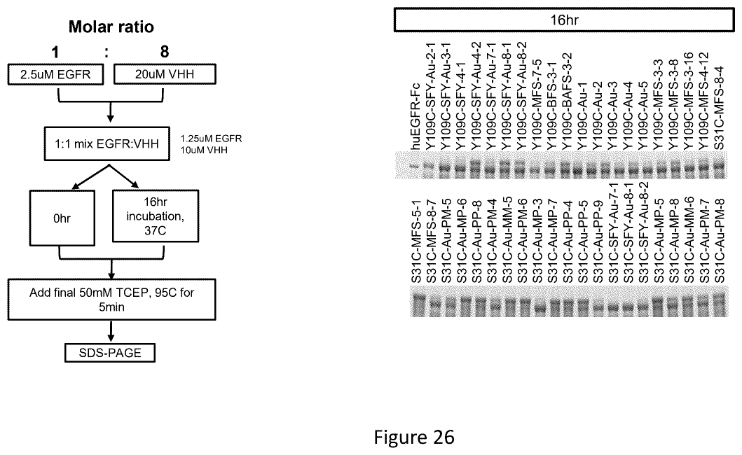

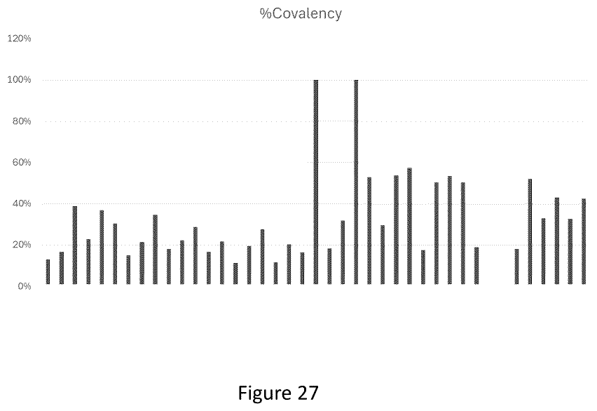

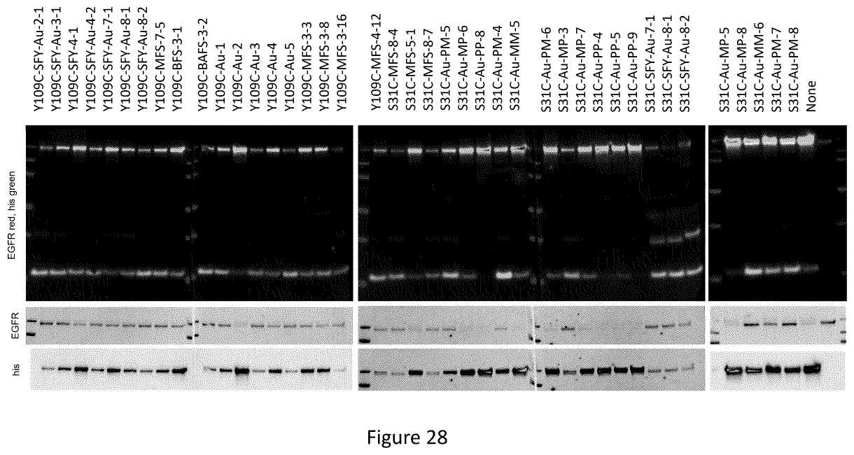

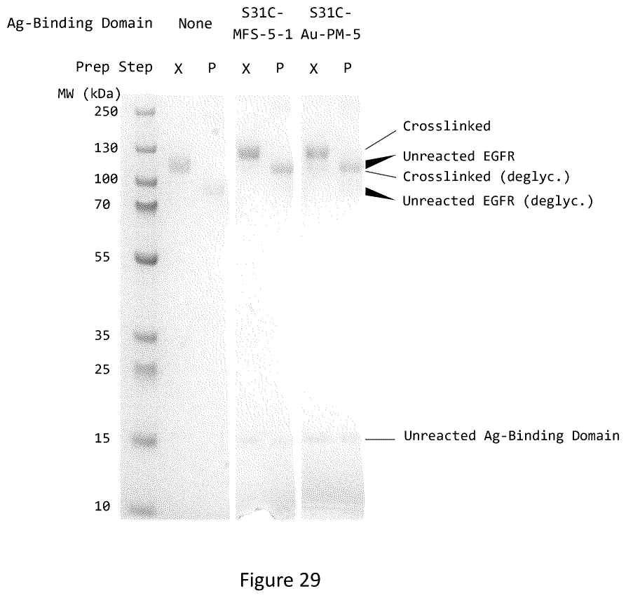

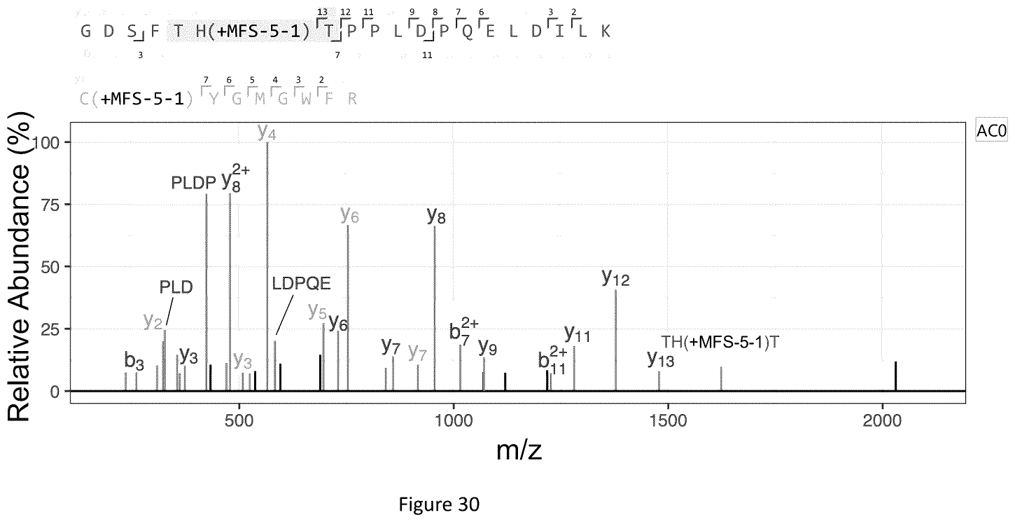

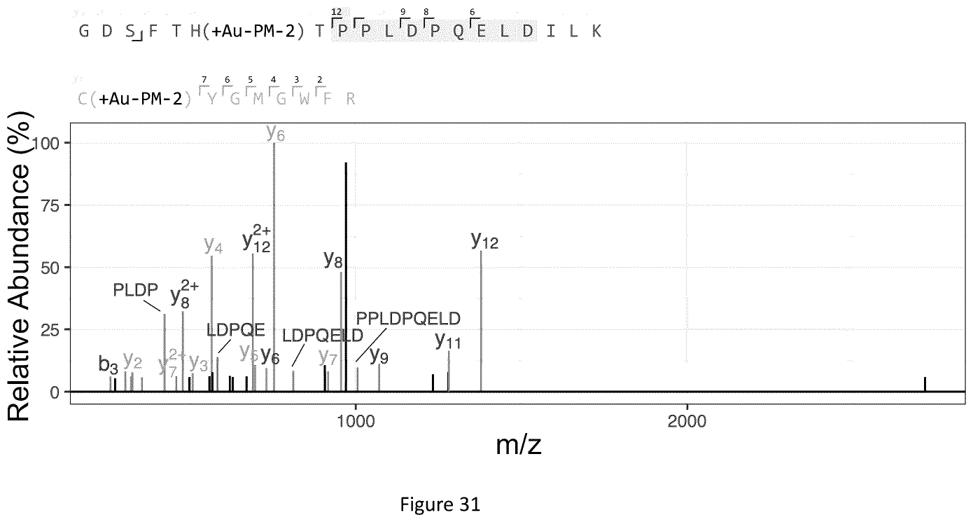

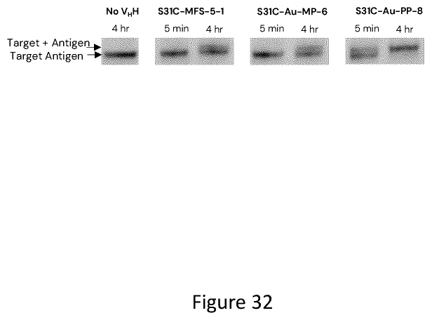

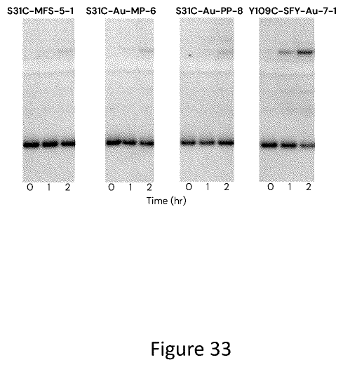

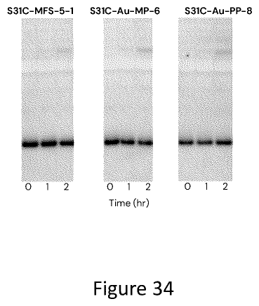

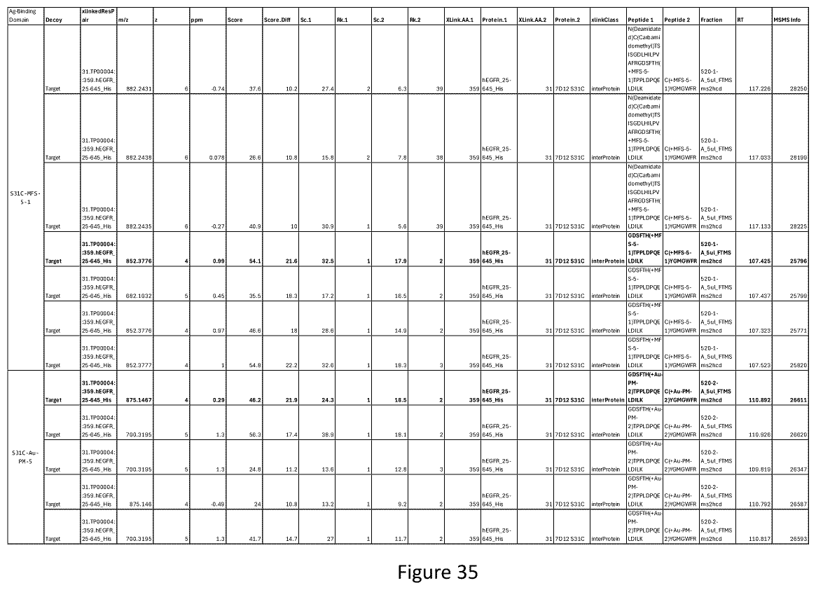

For FIGS. 25 and 26, lane assignments for gel shift assays are provided in the Table below.

| Top Panel Gel Shift Assay - | Bottom Panel Gel Shift Assay - |
|---|---|
| left most lane (top table | left most lane (top table |
| entry) and right most lane | entry) and right most lane |
| (bottom table entry) | (bottom table entry) |
| Y109C-SFY-Au-2-1 | S31C-MFS-5-1 |
| Y109C-SFY-Au-3-1 | S31C-MFS-8-7 |
| Y109C-SFY-4-1 | S31C-Au-PM-5 |
| Y109C-SFY-Au-4-2 | S31C-Au-MP-6 |
| Y109C-SFY-Au-7-1 | S31C-Au-PP-8 |
| Y109C-SFY-Au-8-1 | S31C-Au-PM-4 |
| Y109C-SFY-Au-8-2 | S31C-Au-MM-5 |
| Y109C-MFS-7-5 | S31C-Au-PM-6 |
| Y109C-BFS-3-1 | S31C-Au-MP-3 |
| Y109C-BAFS-3-2 | S31C-Au-MP-7 |
| Y109C-Au-1 | S31C-Au-PP-4 |
| Y109C-Au-2 | S31C-Au-PP-5 |
| Y109C-Au-3 | S31C-Au-PP-9 |
| Y109C-Au-4 | S31C-SFY-Au-7-1 |
| Y109C-Au-5 | S31C-SFY-Au-8-1 |
| Y109C-MFS-3-3 | S31C-SFY-Au-8-2 |
| Y109C-MFS-3-8 | S31C-Au-MP-5 |
| Y109C-MFS-3-16 | S31C-Au-MP-8 |
| Y109C-MFS-4-12 | S31C-Au-MM-6 |
| S31C-MFS-8-4 | S31C-Au-PM-7 |
| | S31C-Au-PM-8 |

FIG. 27 shows a graph of the percent covalency for the antigen-binding domains shown.

FIG. 28 is a Western blot showing covalency after 24 hours between the antigen-binding domains shown and EGFR.

FIG. 29 shows SDS-PAGE analysis of preparative crosslinking reactions prior to digestion for LC-MS/MS.

FIGS. 30 and 31 are mass spectrometry data showing conjugation between an antigen-binding domain and a linker.

FIG. 32 is a Western blot showing crosslinking between the target antigen (EGFR) and the antigen-binding domains shown.

FIGS. 33 and 34 are Western blots showing plasma stability of the complexes shown in FIG. 32 in human plasma.

FIG. 35 shows crosslink spectral matches from tandem mass spectrometry for exemplary antigen-binding domains with linkers after reacting with antigen.

### DETAILED DESCRIPTION

This disclosure provides biomolecules (e.g., a macromolecule, such as a polypeptide or a protein; or a smaller building block thereof (e.g., a peptide or an individual amino acid)), having one or more reactive groups, e.g. having a covalent warhead, and compositions of same, as well as chemical entities that are useful for preparing the biomolecules described herein (any of the linkers described herein; e.g., compounds having formula (A) as described herein; e.g., gold-containing organometallic agents), methods of making and using the proteins and compositions, kits comprising the protein or composition, and methods of screening.

### Gold Complex Embodiments

### Variable R⁶¹

In some embodiments, R⁶¹ is divalent C₆₋₁₀ aryl optionally substituted with 1-4 substituents independently selected from the group consisting of oxo, R^{a}, and R^{b}.

In certain embodiments, R⁶¹ is divalent phenyl optionally substituted with 1-4 substituents independently selected from the group consisting of R^{a} and R^{b}.

In certain embodiments, R⁶¹ is divalent phenyl optionally substituted with 1-4 independently selected R^{a}.

In certain embodiments, R⁶¹ is divalent phenyl optionally substituted with 1-2 independently selected R^{a}.

In certain of the foregoing embodiments, each occurrence of R^{a} is independently selected from the group consisting of halo and C1-10 alkyl which is optionally substituted with 1-6 independently selected R^{d}. For example, wherein each occurrence of R^{a} can be independently selected from the group consisting of fluoro and CH₃.

In some embodiments, R⁶¹ is divalent heteroaryl of 5-10 ring atoms, wherein 1-4 ring atoms are heteroatoms, each independently selected from the group consisting of N, N(H), N(R^{c}), O, and S(O)₀₋₂, and wherein the heteroaryl is optionally substituted with 1-4 substituents independently selected from the group consisting of oxo, R^{a}, and R^{b}.

### Variable R⁶²

In some embodiments, R⁶² is absent.

In other embodiments, R⁶² is C₁-C₁₆ alkylene, C₂-C₁₆ alkenylene, or C₂-C₁₆ alkynylene, each of which is optionally substituted with 1-2 substituents independently selected from halogen and C₃-C₆ cycloalkyl; and wherein 1-8 alkylene units are optionally replaced with a group independently selected from the group consisting of:
(i) —O—;
(ii) —NH—;
(iii) —N(C₁-C₆ alkyl)-;
(iv) —C(O)—;
(v) —S—;
(vi) —S(O)—;
(vii) —S(O)₂—;
(viii) divalent C₃-C₁₀ cycloalkyl, which is optionally substituted with 1-4 R^{a};
(ix) divalent C₆-C₁₀ aryl, which is optionally substituted with 1-4 R^{a};
(x) divalent heteroaryl of 5-10 ring atoms, wherein 1-4 ring atoms are heteroatoms, each independently selected from the group consisting of N, N(H), N(R^{c}), O, and S(O)₀₋₂; and which is optionally substituted with 1-4 R^{a}; and
(xi) divalent heterocyclyl of 3-10 ring atoms, wherein 1-3 ring atoms are heteroatoms, each independently selected from the group consisting of N, N(H), N(R^{c}), N(O⁻), O, and S(O)₀₋₂, and which is optionally substituted with 1-4 R^{a}.

In certain embodiments, R⁶² is C₁-C₁₆ alkylene, which is optionally substituted with 1-2 substituents independently selected from halogen and C₃-C₆ cycloalkyl; and wherein 1-8 alkylene units are each optionally replaced with a group independently selected from the group consisting of:
(i) —O—;
(ii) —NH—;
(iii) —N(C₁-C₆ alkyl)-;
(iv) —C(O)—;
(v) —S—;
(vi) —S(O)—;
(vii) —S(O)₂—;
(viii) divalent C₃-C₁₀ cycloalkyl, which is optionally substituted with 1-4 R^{a};
(ix) divalent phenyl, which is optionally substituted with 1-4 R^{a};
(x) divalent heteroaryl of 5-10 ring atoms, wherein 1-4 ring atoms are heteroatoms, each independently selected from the group consisting of N, N(H), N(R^{c}), O, and S(O)₀₋₂; and which is optionally substituted with 1-4 R^{a}; and
(xi) divalent heterocyclyl of 3-10 ring atoms, wherein 1-3 ring atoms are heteroatoms, each independently selected from the group consisting of N, N(H), N(R^{c}), N(O⁻), O, and S(O)₀₋₂, and which is optionally substituted with 1-4 R^{a}.

In certain embodiments, R⁶² is C₁-C₈ alkylene, which is optionally substituted with 1-2 substituents independently selected from halogen and C₃-C₆ cycloalkyl; and wherein 1-6 alkylene units are each optionally replaced with a group independently selected from the group consisting of:
(i) —O—;
(ii) —NH—;
(iii) —N(C₁-C₆ alkyl)-;
(iv) —C(O)—;
(v) —S—;
(vi) —S(O)—;
(vii) —S(O)₂—;
(viii) divalent C₃-C₁₀ cycloalkyl, which is optionally substituted with 1-4 R^{a};
(ix) divalent phenyl, which is optionally substituted with 1-4 R^{a};
(x) divalent heteroaryl of 5-10 ring atoms, wherein 1-4 ring atoms are heteroatoms, each independently selected from the group consisting of N, N(H), N(R^{c}), O, and S(O)₀₋₂; and which is optionally substituted with 1-4 R^{a}; and
(xi) divalent heterocyclyl of 3-10 ring atoms, wherein 1-3 ring atoms are heteroatoms, each independently selected from the group consisting of N, N(H), N(R^{c}), N(O⁻), O, and S(O)₀₋₂, and which is optionally substituted with 1-4 R^{a}.

In certain embodiments, R⁶² is C₁-C₈ alkylene, which is optionally substituted with 1-2 substituents independently selected from halogen and C₃-C₆ cycloalkyl; and wherein 1-6 alkylene units are each optionally replaced with a group independently selected from the group consisting of:
(i) —O—;
(ii) —NH—;
(iii) —N(C₁-C₆ alkyl)-;
(iv) —C(O)—;
(viii) divalent C₃-C₁₀ cycloalkyl, which is optionally substituted with 1-4 R^{a};
(ix) divalent phenyl, which is optionally substituted with 1-4 R^{a}; and
(xi) divalent heterocyclyl, wherein 1-3 ring atoms are heteroatoms, each independently selected from the group consisting of N, N(H), N(R^{c}), N(O⁻), O, and S(O)₀₋₂, and which is optionally substituted with 1-4 R^{a}.

In certain embodiments, R⁶² has formula (II):

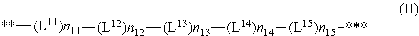

wherein ** indicates the point of attachment of formula (II) to R⁶¹, and *** indicates the point of attachment of formula (II) to the reactive group;
wherein each of n₁₁, n₁₂, n₁₃, n₁₄, and n₁₅ is independently 0 or 1, provided that at least one of n₁₁, n₁₂, n₁₃, n₁₄, and n₁₅ is 1; and
each occurrence of L¹¹, L¹², L¹³, L¹⁴, and L¹⁵ is independently selected from the group consisting of:
(i) —O—;
(ii) —NH—;
(iii) —N(C₁-C₆ alkyl)-;
(iv) —C(O)—;
(v) —S—;
(vi) —S(O)—;
(vii) —S(O)₂—;
(viii) divalent C₃-C₁₀ cycloalkyl, which is optionally substituted with 1-4 R^{a};
(ix) divalent phenyl, which is optionally substituted with 1-4 R^{a};
(x) divalent heteroaryl of 5-10 ring atoms, wherein 1-4 ring atoms are heteroatoms, each independently selected from the group consisting of N, N(H), N(R^{c}), O, and S(O)₀₋₂; and which is optionally substituted with 1-4 R^{a};
(xi) divalent heterocyclyl of 3-10 ring atoms, wherein 1-3 ring atoms are heteroatoms, each independently selected from the group consisting of N, N(H), N(R^{c}), N(O⁻), O, and S(O)₀₋₂, and which is optionally substituted with 1-4 R^{a}; and
(xi) C₁-C₂ alkylene.

In some formula (II) embodiments, each occurrence of L¹¹, L¹², L¹³, L¹⁴, and L¹⁵ is independently selected from the group consisting of:
(i) —O—;
(ii) —NH—;
(iii) —N(C₁-C₆ alkyl)-;
(iv) —C(O)—;
(viii) divalent C₃-C₁₀ cycloalkyl, which is optionally substituted with 1-4 R^{a};
(ix) divalent phenyl, which is optionally substituted with 1-4 R^{a};
(xi) divalent heterocyclyl of 3-10 ring atoms, wherein 1-3 ring atoms are heteroatoms, each independently selected from the group consisting of N, N(H), N(R^{c}), N(O⁻), O, and S(O)₀₋₂, and which is optionally substituted with 1-4 R^{a}; and
(xi) C₁-C₂ alkylene.

In certain formula (II) embodiments, n₁₁ is 1.

In certain of these formula (II) embodiments, L¹¹ is —NH—.

In other of these formula (II) embodiments, L¹¹ is —O—.

In still other of these embodiments, L¹¹ is —CH₂—.

In still other of these embodiments, L¹¹ is —C(O)—.

In certain formula (II) embodiments, n₁₅ is 1.

In certain of these formula (II) embodiments, L¹⁵ is divalent phenyl, which is optionally substituted with 1-4 R^{a}.

For example, L¹⁵ is unsubstituted divalent phenyl.

In certain formula (II) embodiments, one of n₁₂, n₁₃, and n₁₄ is 1, and the others are 0. In certain formula (II) embodiments, two of n₁₂, n₁₃, and n₁₄ are 1, and the other is 0.

In certain formula (II) embodiments, each of n₁₂, n₁₃, and n₁₄ is 1.

In certain formula (II) embodiments, each of n₁₂, n₁₃, and n₁₄ is 0.

In certain of these formula (II) embodiments, each of L¹², L³, and L¹⁴ is independently selected from the group consisting of:
(iv) —C(O)—;
(viii) divalent C₃-C₁₀ cycloalkyl, which is optionally substituted with 1-4 R^{a};
(ix) divalent phenyl, which is optionally substituted with 1-4 R^{a};
(xi) divalent heterocyclyl of 3-10 ring atoms, wherein 1-3 ring atoms are heteroatoms, each independently selected from the group consisting of N, N(H), N(R′), N(O⁻), O, and S(O)₀₋₂, and which is optionally substituted with 1-4 R^{a}; and
(xi) C₁-C₂ alkylene.

For example, each of L², L³, and L¹⁴, when present, can each independently be selected from the group consisting of —C(O)—, divalent cyclohexyl, and divalent piperidinyl.

In certain formula (II) embodiments, n₁₁ is 1, and n₁₅ is 1.

In certain of these formula (II) embodiments, L¹¹ is —NH—.

In certain of these formula (II) embodiments, L¹¹ is —O—.

In certain of these formula (II) embodiments, L¹¹ is —CH₂—.

In certain of these formula (II) embodiments, L¹¹ is —C(O)—.

In certain of the foregoing formula (II) embodiments, L⁵ is divalent phenyl, which is optionally substituted with 1-4 R^{a}. For example, L¹⁵ can be unsubstituted divalent phenyl.

In certain of the foregoing formula (II) embodiments, one, two, or three of n¹², n₁₃, and n₁₄ are 1, and the others are 0.

In certain of the foregoing formula (II) embodiments, each of n₁₂, n₁₃, and n₁₄ is 0.

In other formula (II) embodiments, each of L¹², L¹³, and L¹⁴ is independently selected from the group consisting of:
(iv) —C(O)—;
(viii) divalent C₃-C₁₀ cycloalkyl, which is optionally substituted with 1-4 R^{a};
(ix) divalent phenyl, which is optionally substituted with 1-4 R^{a};
(xi) divalent heterocyclyl of 3-10 ring atoms, wherein 1-3 ring atoms are heteroatoms, each independently selected from the group consisting of N, N(H), N(R′), N(O⁻), O, and S(O)₀₋₂, and which is optionally substituted with 1-4 R^{a}; and
(xi) C₁-C₂ alkylene.

For example, each of L², L¹³, and L⁴ is independently selected from the group consisting of —C(O)—, divalent cyclohexyl, and divalent piperidinyl.

In certain formula (II) embodiments:
n₁₁ is 1, and n₁₅ is 1; L¹¹ is —NH—. —O—, —CH₂—, or —C(O)—; optionally wherein L¹¹ is —NH— or —O—;
L¹⁵ is divalent phenyl, which is optionally substituted with 1-4 R^{a}; optionally wherein, L¹⁵ is unsubstituted divalent phenyl; and each of nu12, n₁₃, and n₁₄ is 0.

In certain formula (II) embodiments:
n₁₁ is 1, and n₁₅ is 1; L¹¹ is —NH—. —O—, —CH₂—, or —C(O)—; optionally wherein L¹¹ is —NH— or —O—;
L¹⁵ is divalent phenyl, which is optionally substituted with 1-4 R^{a}; optionally wherein, L¹⁵ is unsubstituted divalent phenyl;
one, two, or three of n₁₂, n₁₃, and n₁₄ are 1, and the others are 0; and each of L², L¹³, and L¹⁴ is independently selected from the group consisting of: (iv) —C(O)—;
(viii) divalent C₃-C₁₀ cycloalkyl, which is optionally substituted with 1-4 R^{a};
(ix) divalent phenyl, which is optionally substituted with 1-4 R^{a};
(xi) divalent heterocyclyl of 3-10 ring atoms, wherein 1-3 ring atoms are heteroatoms, each independently selected from the group consisting of N, N(H), N(RC), N(O⁻), O, and S(O)₀₋₂, and which is optionally substituted with 1-4 R^{a}; and
(xi) C₁-C₂ alkylene.

For example, each of L¹², L¹³, and L¹⁴ can be independently selected from the group consisting of —C(O)—, divalent cyclohexyl, and divalent piperidinyl.

### Variable R⁶³

Variable R⁶³ is a reactive group.

Non-limiting examples of reactive groups include:
1) α,β unsaturated systems (e.g., L^{W1}-EWG, wherein L^{W1} is alkenyl or alkynyl; and EWG is an electron withdrawing group; e.g., Michael acceptors, e.g., acrylamides, acrylates, vinylsulfones, α,β-unsaturated ketones, and the like)
2) Strained heterocycles (e.g., heterocycles including from 3-4 ring atoms wherein 1 ring atom is a heteroatom selected from oxygen, nitrogen, and sulfur; e.g., epoxide, aziridine, beta-lactam, and other strained systems);
3) Strained carbocyclic systems (e.g., cyclopropyl substituted with one or more electron-withdrawing groups);
4) Electron-deficient arenes/heteroarenes (e.g., pyridine or fluorobenzene) which can undergo S_{N}Ar reaction (e.g., with cysteine or lysine);
5) Sulfur-containing heteroarenes (e.g., thiadiazole);
6) Styrenyl moieties (i.e., aryl/heteroaryl that is directly conjugated to an alkenyl or alkynyl);
7) Activated ketone (e.g., halomethylketone);
8) Acylating agents (e.g., carbamates, aza-peptides, acyl hydroxamates);
9) phosphonylating agents (e.g., phosphonyl fluorides), 10) sulfonylation agents (e.g., sulfonyl fluoride, e.g., —OSO₂—F or sulfonyl-HET, e.g., —OSO₂—HET, in which HET is an optionally substituted heteroaryl of 5-10 ring atoms, wherein from 1-4 ring atoms are heteroatoms, each independently selected from the group consisting of N and N(H); and wherein the heteroaryl is linked to the sulfur atom by a ring nitrogen atom);
11) Aldehydes;
12) Boronic acids or boronic esters; and
13) Organonitrile compounds (e.g., alkyl nitrile, cyanamide, or acyl cyanamide). Exemplary reactive groups include, but are not limited to:

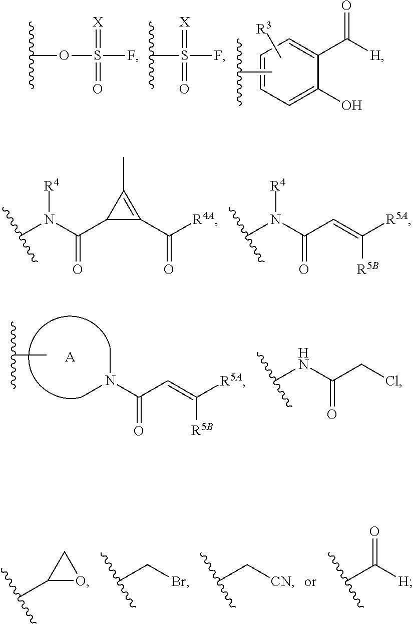

X is O or NR^{X};
and R^{X} is hydrogen, C1-C6 alkyl, or C3-C6 cycloalkyl.

In certain embodiments, R⁶³ is

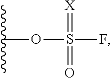

wherein X is O.

In certain embodiments, R⁶³ is sulfonyl-HET, e.g., —OSO₂—HET, in which HET is an optionally substituted heteroaryl of 5-10 ring atoms, wherein from 1-4 ring atoms are heteroatoms, each independently selected from the group consisting of N and N(H); and wherein the heteroaryl is linked to the sulfur atom by a ring nitrogen atom. In other embodiments, HET can further include O and/or S.

In certain of the foregoing embodiments, HET is an optionally substituted heteroaryl of 5-6 ring atoms, wherein from 1-4 ring atoms are heteroatoms, each independently selected from the group consisting of N and N(H); and wherein the heteroaryl is linked to the sulfur atom by a ring nitrogen atom.

In certain of the foregoing embodiments, HET is an optionally substituted heteroaryl of 5 ring atoms, wherein from 1-4 ring atoms are heteroatoms, each independently selected from the group consisting of N and N(H); and wherein the heteroaryl is linked to the sulfur atom by a ring nitrogen atom. For example, HET can be pyrrolyl, imidazolyl, triazolyl, pyrazolyl, or tetrazolyl.

In certain of the foregoing embodiments, HET is an optionally substituted heteroaryl of 6 ring atoms, wherein from 1-2 ring atoms are heteroatoms, each independently selected from the group consisting of N and N(H); and wherein the heteroaryl is linked to the sulfur atom by a ring nitrogen atom. For example, HET can be 6-member aromatic lactams, aromatic cyclic ureas, or vinylogous analogs thereof, in which each ring nitrogen adjacent to a carbonyl is tertiary (i.e., all three valences are occupied by non-hydrogen substituents), such as one or more of pyridone

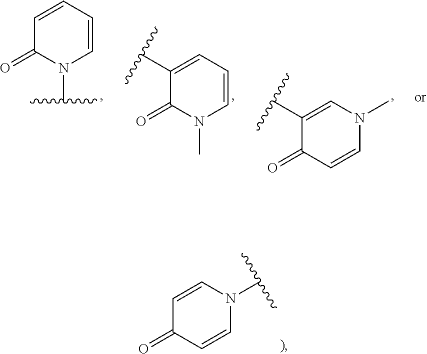

pyrimidone

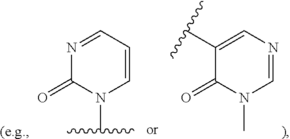

pyridazinone

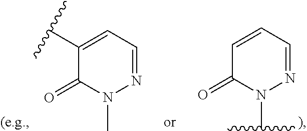

pyrazinone

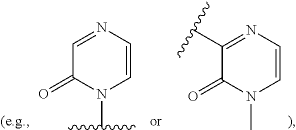

and imidazolone

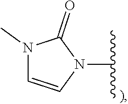 [SMILES: *n1ccn(C)c1=O]

wherein each ring nitrogen adjacent to a carbonyl is tertiary (i.e., the oxo group (i.e., “═O”) herein is a constituent part of the heteroaryl ring).

Non-limiting examples of sulfonyl-HET are provided below (here, for illustrative purposes only, as a substituent present on a gold complex).

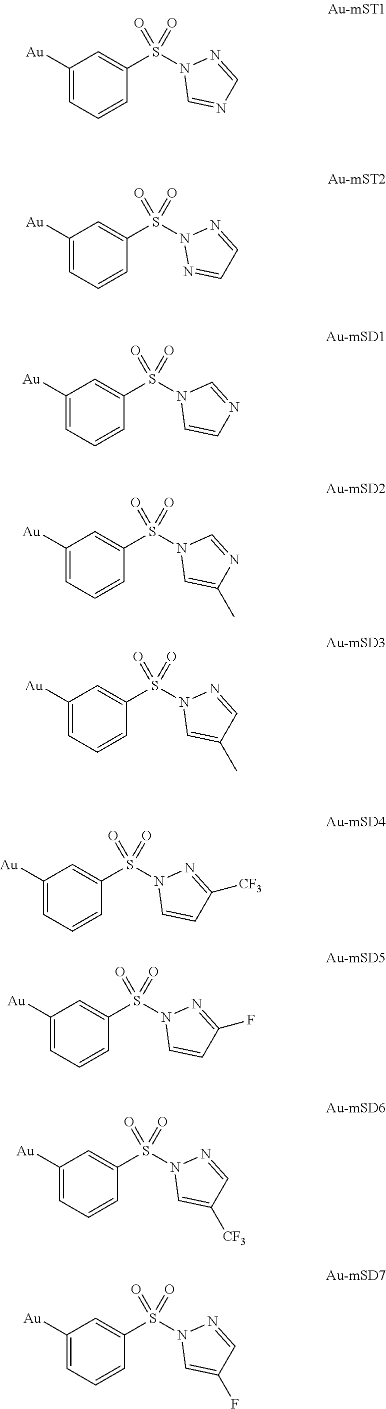

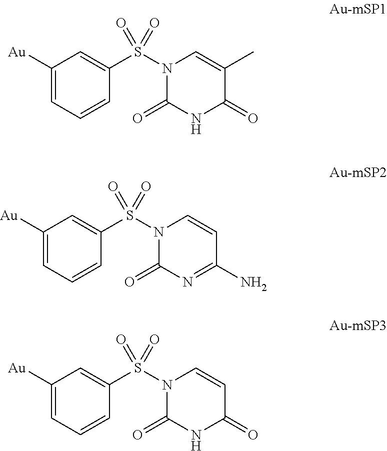

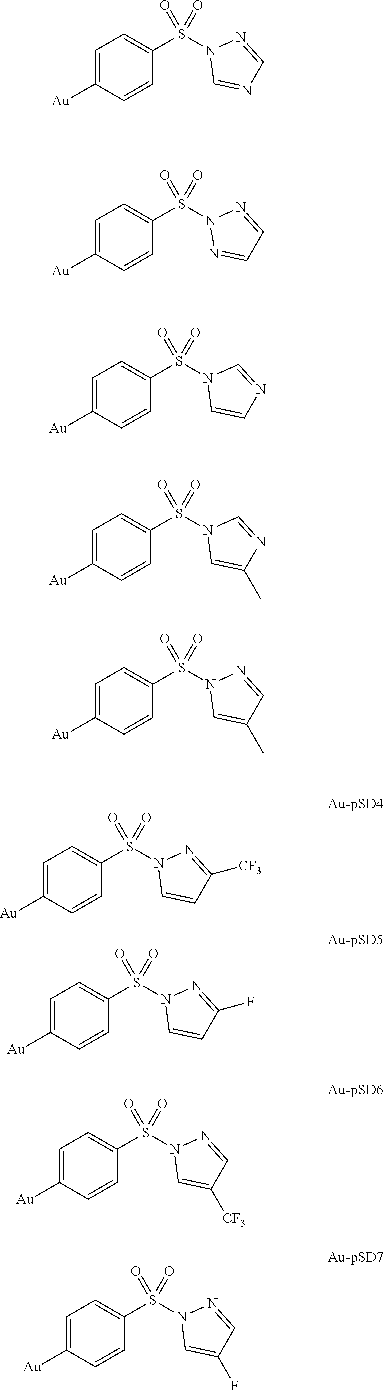

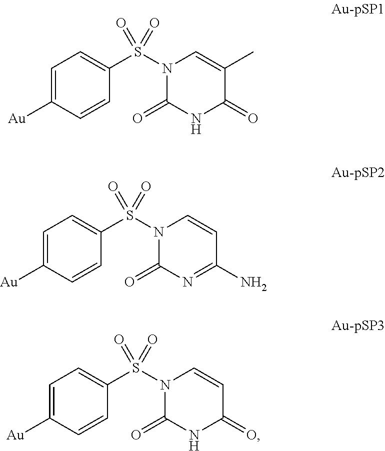

Variables Ring a, R¹ and R² and R³ and R⁴

In some embodiments, Ring A is C₆₋₁₄ aryl optionally substituted with 1-4 substituents independently selected from the group consisting of oxo, R^{a}, and R^{b}.

In certain embodiments, Ring A is phenyl optionally substituted with 1-4 substituents independently selected from the group consisting of oxo, R^{a}, and R^{b}.

In some embodiments, each of R¹ and R² is independently C₃₋₁₂ cycloalkyl optionally substituted with substituents independently selected from the group consisting of oxo, R^{a}, and R^{b}.

In certain embodiments, each of R¹ and R² is independently C₅₋₇ cycloalkyl optionally substituted with substituents independently selected from the group consisting of oxo, R^{a}, and R^{b}.

In certain embodiments, each of R¹ and R² is independently C₆ cycloalkyl optionally substituted with substituents independently selected from the group consisting of oxo, R^{a}, and R^{b}.

In some embodiments, each of R³ and R⁴ is independently C₁₋₁₀ alkyl optionally substituted with 1-4 independently selected R^{d}.

In certain embodiments, each of R³ and R⁴ is CH₃.

### Variable R⁵

In some embodiments, R⁵ is monovalent (i.e., including one formal negative charge). An anionic counterion may also be multivalent (i.e., including more than one formal negative charge), such as divalent or trivalent. Exemplary monovalent anions include halide ions (e.g., F⁻, Cl⁻, Br⁻, I⁻), NO₃⁻, ClO₄⁻, OH⁻, H₂PO₄, HCO₃⁻, HSO₄⁻, sulfonate ions (e.g., methansulfonate, trifluoromethanesulfonate, p-toluenesulfonate, benzenesulfonate, 10-camphor sulfonate, naphthalene-2-sulfonate, naphthalene-1-sulfonic acid-5-sulfonate, ethan-1-sulfonic acid-2-sulfonate), carboxylate ions (e.g., acetate, propanoate, benzoate, glycerate, lactate, tartrate, glycolate, gluconate), BF₄⁻, PF₄⁻, PF₆⁻, AsF₆⁻, SbF₆⁻, B[3,5-(CF₃)₂C₆H₃]₄]⁻, B(C₆F₅)₄⁻, BPh4⁻, Al(OC(CF₃)₃)₄⁻, and carborane anions (e.g., CB₁₁H₁₂⁻ or (HCB₁₁Me₅Br₆)⁻). Exemplary anions which may be multivalent include CO₃²⁻, HPO₄²⁻ PO₄³⁻, B₄O₇²⁻, SO₄²⁻, S₂O₃²⁻, carboxylate anions (e.g., tartrate, citrate, fumarate, maleate, malate, malonate, gluconate, succinate, glutarate, adipate, pimelate, suberate, azelate, sebacate, salicylate, phthalates, aspartate, glutamate, and the like), and carboranes.

In some embodiments, R⁵ is chloro.

### Non-Limiting Combinations

### [A]

In some embodiments:
R⁶¹ is divalent C₆₋₁₀ aryl optionally substituted with 1-4 substituents independently selected from the group consisting of oxo, R^{a}, and R^{b}; e.g., R⁶¹ is divalent phenyl optionally substituted with 1-4 substituents independently selected from the group consisting of R^{a} and R^{b} and
R⁶² is absent;
In some embodiments of [A], R⁶³ is:

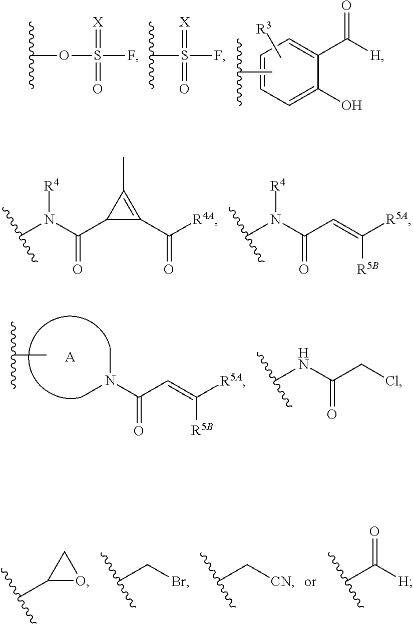

X is O or NR^{X}; and R^{X} is hydrogen, C₁-C₆ alkyl, or C₃-C₆ cycloalkyl.

In certain embodiments of [A], R⁶³ is

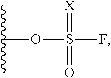

wherein X is O.

In certain embodiments of [A], R⁶³ is —OSO₂—HET.

In some embodiments of [A], R⁵ is chloro.

In some embodiments of [A], each of R¹ and R² is independently C₅₋₇ cycloalkyl optionally substituted with substituents independently selected from the group consisting of oxo, R^{a}, and R^{b}; e.g., each of R¹ and R² is independently C₆ cycloalkyl optionally substituted with substituents independently selected from the group consisting of oxo, R^{a}, and R^{b}.

In some embodiments of [A], each of R³ and R⁴ is independently C₁₋₁₀ alkyl optionally substituted with 1-4 independently selected R^{d}; e.g., each of R³ and R⁴ is CH₃.

In some embodiments of [A], Ring A is phenyl optionally substituted with 1-4 substituents independently selected from the group consisting of oxo, R^{a}, and R^{b}.

### [B]

In some embodiments:
R⁶¹ is divalent C₆₋₁₀ aryl optionally substituted with 1-4 substituents independently selected from the group consisting of oxo, R^{a}, and R^{b}; e.g., R⁶¹ is divalent phenyl optionally substituted with 1-4 substituents independently selected from the group consisting of R^{a} and R^{b} and
R⁶² is C₁-C₁₆ alkylene, C₂-C₁₆ alkenylene, or C₂-C₁₆ alkynylene, each of which is optionally substituted with 1-2 substituents independently selected from halogen and C₃-C₆ cycloalkyl; and wherein 1-8 alkylene units are optionally replaced with a group independently selected from the group consisting of:
(i) —O—;
(ii) —NH—;
(iii) —N(C₁-C₆ alkyl)-;
(iv) —C(O)—;
(v) —S—;
(vi) —S(O)—;
(vii) —S(O)₂—;
(viii) divalent C₃-C₁₀ cycloalkyl, which is optionally substituted with 1-4 R^{a};
(ix) divalent C₆-C₁₀ aryl, which is optionally substituted with 1-4 R^{a};
(x) divalent heteroaryl of 5-10 ring atoms, wherein 1-4 ring atoms are heteroatoms, each independently selected from the group consisting of N, N(H), N(R^{c}), O, and S(O)₀₋₂; and which is optionally substituted with 1-4 R^{a}; and
(xi) divalent heterocyclyl of 3-10 ring atoms, wherein 1-3 ring atoms are heteroatoms, each independently selected from the group consisting of N, N(H), N(R^{c}), N(O⁻), O, and S(O)₀₋₂, and which is optionally substituted with 1-4 R^{a}.

In some embodiments of [B], R⁶² has formula (II): In some embodiments of [B], n₁₁ is 1.

In some embodiments of [B], L¹¹ is —NH—.

In some embodiments of [B], L¹¹ is —O—.

In some embodiments of [B], L¹¹ is —CH₂—.

In some embodiments of [B], L¹¹ is —C(O)—.

In some embodiments of [B], n₁₅ is 1.

In some embodiments of [B], L¹⁵ is divalent phenyl, which is optionally substituted with 1-4 R^{a}. For example, L¹⁵ is unsubstituted divalent phenyl.

In some embodiments of [B], one of nu12, n₃, and n₁₄ is 1, and the others are 0.

In some embodiments of [B], two of nu12, n₁₃, and n₁₄ are 1, and the other is 0.

In some embodiments of [B], each of n₁₂, n₁₃, and n₁₄ is 1.

In some embodiments of [B], each of n₁₂, n₁₃, and n₁₄ is 0.

In some embodiments of [B], each of L², L¹³, and L¹⁴ is independently selected from the group consisting of:
(iv) —C(O)—;
(viii) divalent C₃-C₁₀ cycloalkyl, which is optionally substituted with 1-4 R^{a};
(ix) divalent phenyl, which is optionally substituted with 1-4 R^{a};
(xi) divalent heterocyclyl of 3-10 ring atoms, wherein 1-3 ring atoms are heteroatoms, each independently selected from the group consisting of N, N(H), N(R^{c}), N(O⁻), O, and S(O)₀₋₂, and which is optionally substituted with 1-4 R^{a}; and
(xi) C₁-C₂ alkylene.

For example, each of L¹², L¹³, and L¹⁴, when present, can each independently be selected from the group consisting of —C(O)—, divalent cyclohexyl, and divalent piperidinyl.

In some embodiments of [B], R⁶³ is:

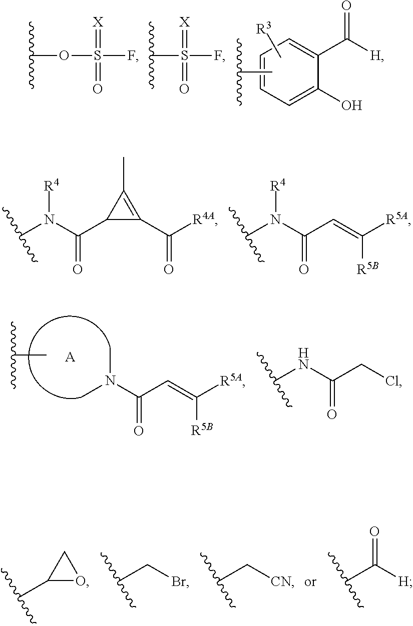

### X is O or NR^{X};

and R^{X} is hydrogen, C₁-C₆ alkyl, or C₃-C₆ cycloalkyl.

In certain embodiments of [B], R⁶³ is

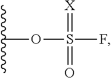

wherein X is O.

In certain embodiments of [B], R⁶³ is —OSO₂—HET.

In some embodiments of [B], R⁵ is chloro.

In some embodiments of [B], each of R¹ and R² is independently C₅₋₇ cycloalkyl optionally substituted with substituents independently selected from the group consisting of oxo, R^{a}, and R^{b}; e.g., each of R¹ and R² is independently C₆ cycloalkyl optionally substituted with substituents independently selected from the group consisting of oxo, R^{a}, and R^{b}.

In some embodiments of [B], each of R³ and R⁴ is independently C₁₋₁₀ alkyl optionally substituted with 1-4 independently selected R^{d}; e.g., each of R³ and R⁴ is CH₃.

In some embodiments of [B], Ring A is phenyl optionally substituted with 1-4 substituents independently selected from the group consisting of oxo, R^{a}, and R^{b}.

In some embodiments, the compound of formula (I) is selected from a compound in Table L. In some embodiments, the protein is modified by a chemical linker selected from Table L.

TABLE L
Exemplary Linkers

| Example | ID | Structure |
|---|---|---|
| 1 | Au-1-3-Me | 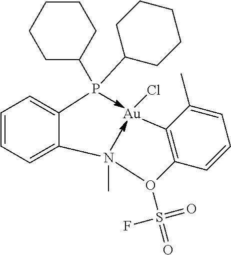 |
| 2 | Au-1-4-F | 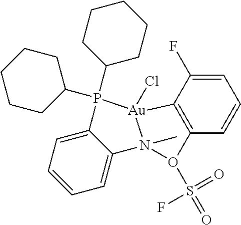 |
| 3 | Au-2-2-CN | 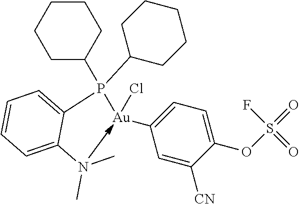 |
| 4 | Au-3-1-Ome | 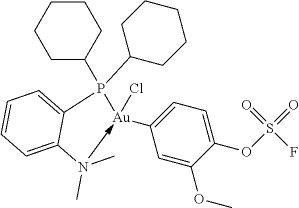 |
| 5 | Au-3-2-CN | 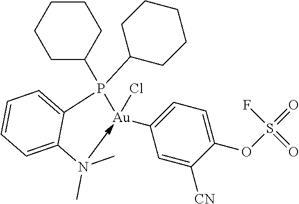 |
| 6 | Au-MP-7 | 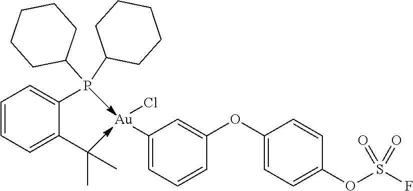 |
| 7 | Au-PM-2 | 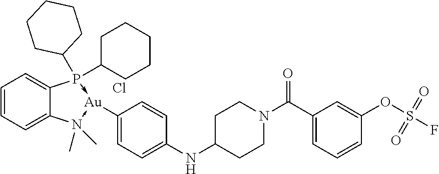 |
| 8 | Au-PP-10 | 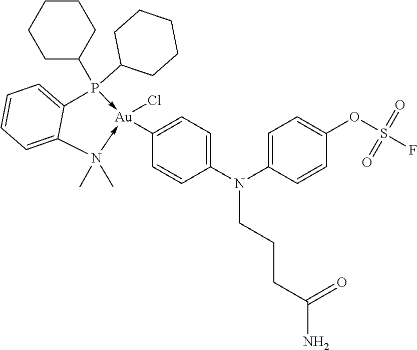 |
| 9 | SFY-Au-7-1 | 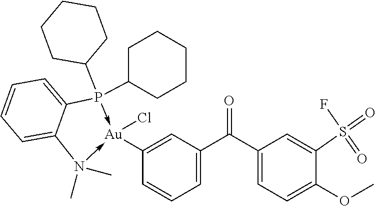 |
| 10 | SFY-Au-8-1 | 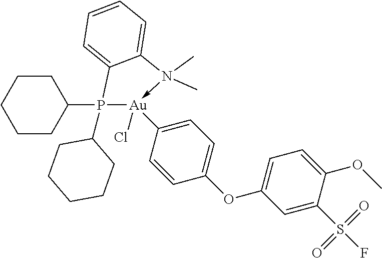 |
| 11 | SFY-Au-8-2 | 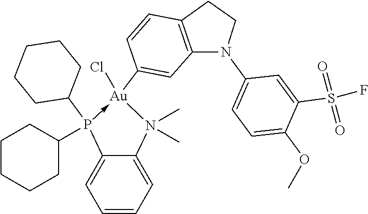 |
| 12 | Au-1-1-OMe | 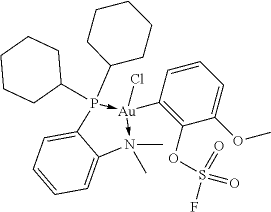 |
| 13 | Au-2-1-OMe | 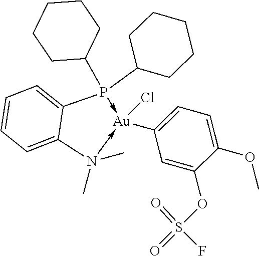 |
| 14 | Au-5-1-Me | 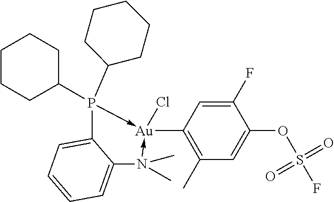 |
| 15 | Au-6 | 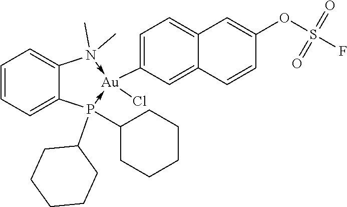 |
| 16 | Au-7 | 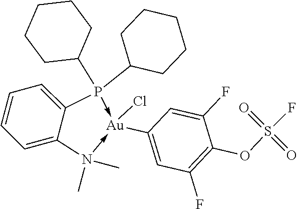 |
| 17 | Au-8 | 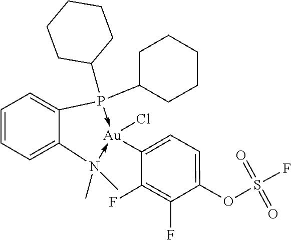 |
| 18 | Au-MM-1 | 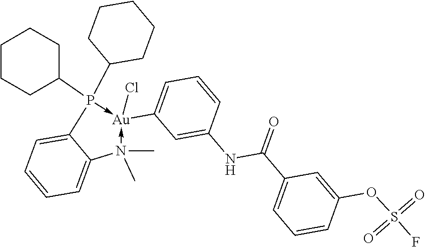 |
| 19 | Au-MP-4 | 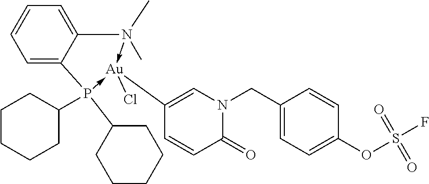 |
| 20 | Au-MP-5 | 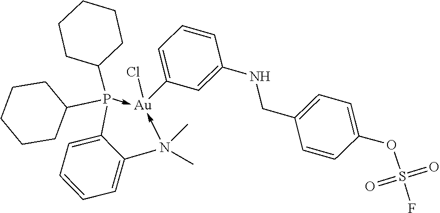 |
| 21 | Au-MP-6 | 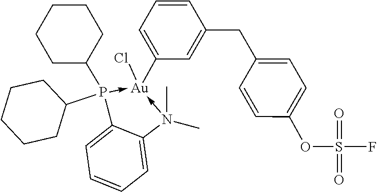 |
| 22 | Au-MP-8 | 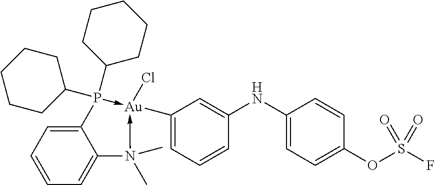 |
| 23 | Au-PM-3A | 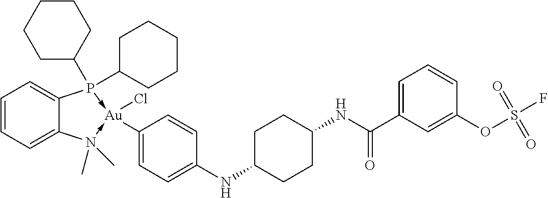 |
| 23 | Au-PM-3B | 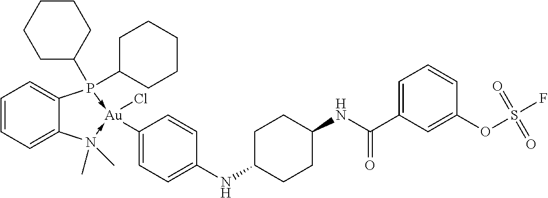 |
| 24 | Au-PM-5 | 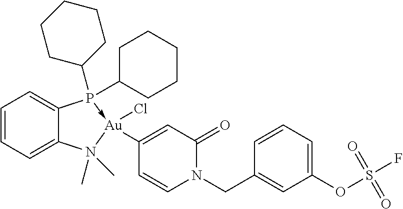 |
| 25 | Au-PM-7 | 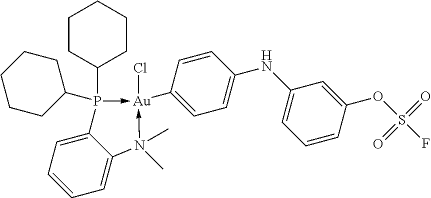 |
| 26 | Au-PP-3A | 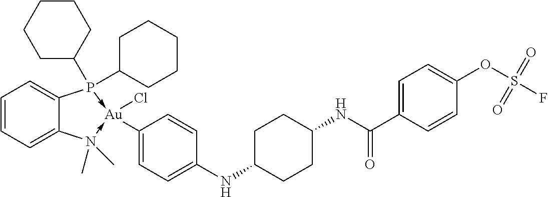 |
| 26 | Au-PP-3B | 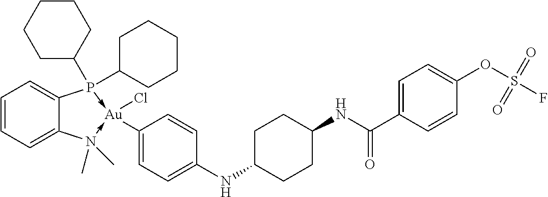 |
| 27 | Au-PP-9 | 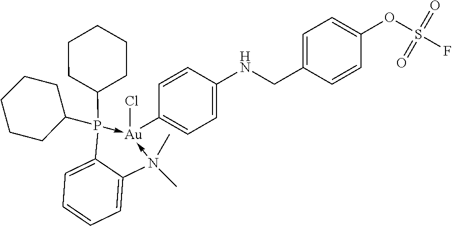 |
| 28 | SFY-Au-2-1 | 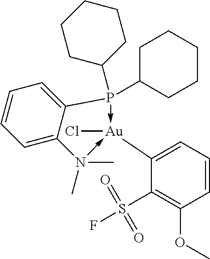 |
| 29 | SFY-Au-3-1 | 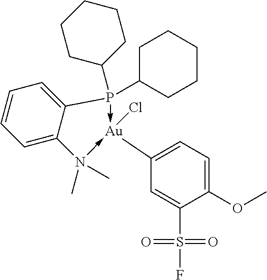 |
| 30 | Au-PM-8 | 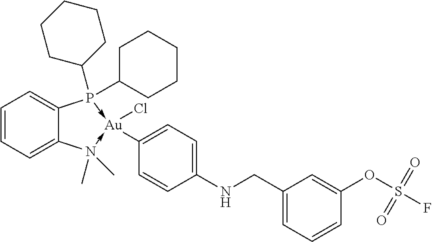 |
| 31 | Au-PP-8 | 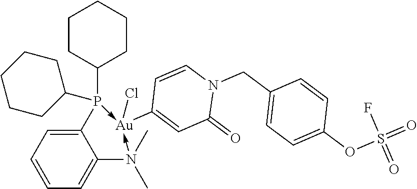 |
| 32 | Au-PP-1 | 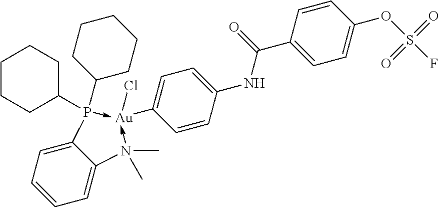 |
| 33 | Au-Pyr-1 | 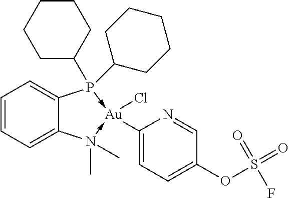 |
| 34 | Au-01 | 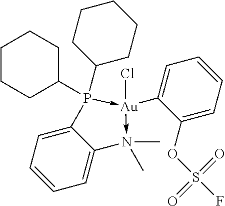 |
| 35 | Au-02 | 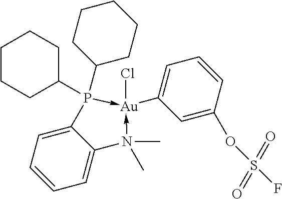 |
| 36 | Au-03 | 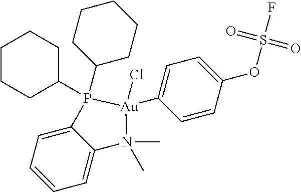 |
| 37 | Au-04 | 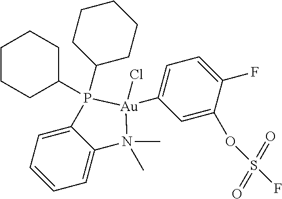 |
| 38 | Au-05 | 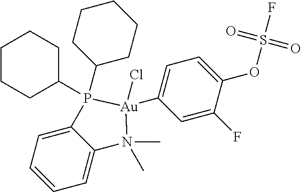 |
| 39 | Au-8 | 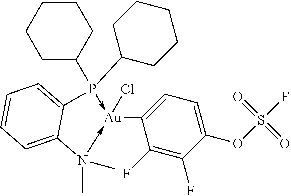 |
| 40 | Au-4-1-Me | 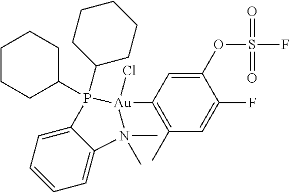 |
| 41 | Au-2-6-F | 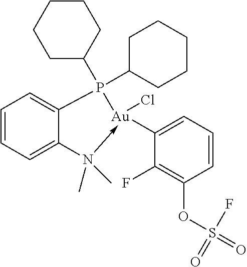 |
| 42 | Au-MP-1 | 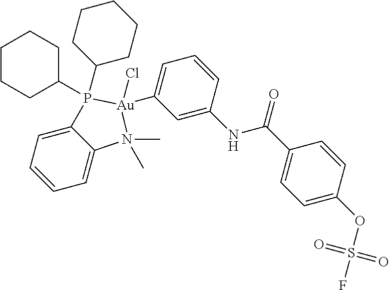 |
| 43 | Au-MP-3 | 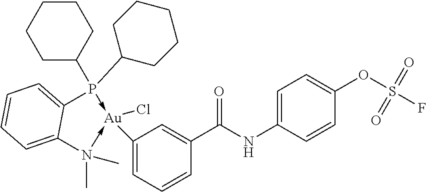 |
| 44 | Au-MM-2 | 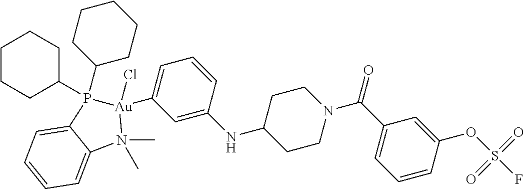 |
| 45 | Au-MM-5 | 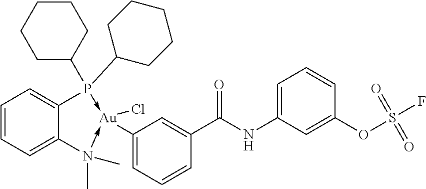 |
| 46 | Au-MM-6 | 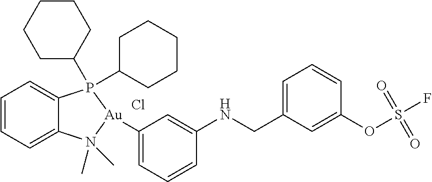 |
| 47 | Au-PM-1 | 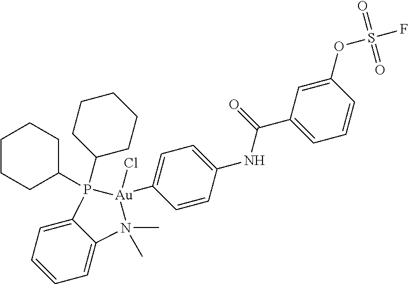 |
| 48 | Au-PM-4 | 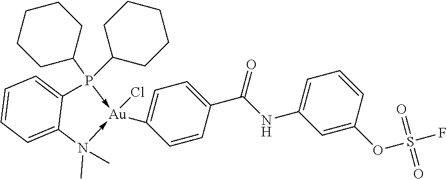 |
| 49 | Au-PM-6 | 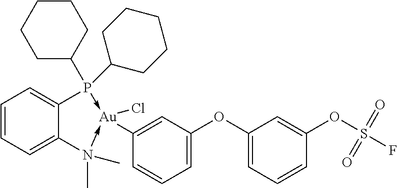 |
| 50 | Au-PP-4 | 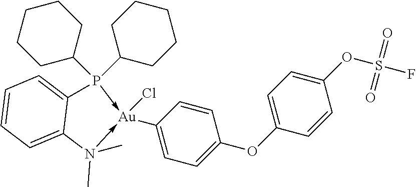 |
| 51 | Au-PP-5 | 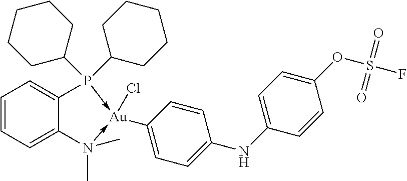 |
| 52 | MFS-3-3 | 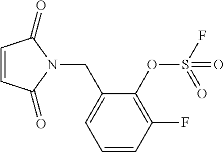 [SMILES: O=C1C=CC(=O)N1Cc1cccc(F)c1OS(=O)(=O)F] |
| 52 | MFS-3-3 | 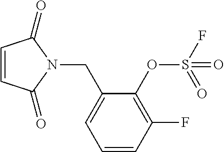 [SMILES: O=C1C=CC(=O)N1Cc1cccc(F)c1OS(=O)(=O)F] |
| 53 | MFS-4-2 | 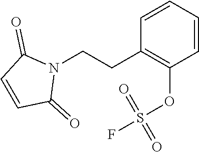 [SMILES: O=C1C=CC(=O)N1CCc1ccccc1OS(=O)(=O)F] |
| 54 | MFS-4-4 | 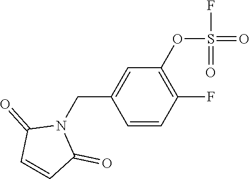 [SMILES: O=C1C=CC(=O)N1Cc1ccc(F)c(OS(=O)(=O)F)c1] |
| 55 | MFS-6-2 | 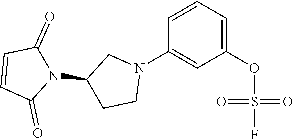 [SMILES: O=C1C=CC(=O)N1[C@@H]1CCN(c2cccc(OS(=O)(=O)F)c2)C1] |
| 56 | MFS-6-3 | 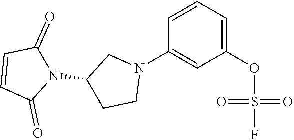 [SMILES: O=C1C=CC(=O)N1[C@H]1CCN(c2cccc(OS(=O)(=O)F)c2)C1] |
| 57 | MFS-7-1 | 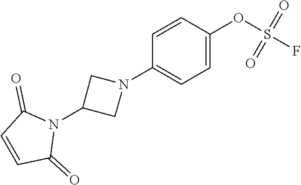 [SMILES: O=C1C=CC(=O)N1C1CN(c2ccc(OS(=O)(=O)F)cc2)C1] |
| 58 | MFS-7-2 | 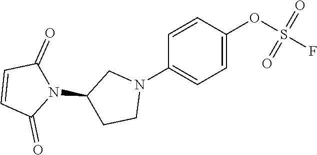 [SMILES: O=C1C=CC(=O)N1[C@@H]1CCN(c2ccc(OS(=O)(=O)F)cc2)C1] |
| 59 | MFS-7-4 | 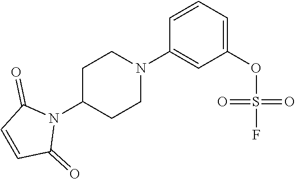 [SMILES: O=C1C=CC(=O)N1C1CCN(c2cccc(OS(=O)(=O)F)c2)CC1] |
| 60 | MFS-7-5 | 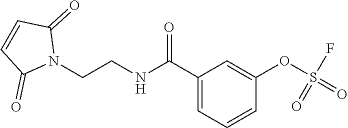 [SMILES: O=C(NCCN1C(=O)C=CC1=O)c1cccc(OS(=O)(=O)F)c1] |
| 61 | MFS-8-2 | 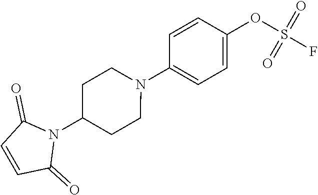 [SMILES: O=C1C=CC(=O)N1C1CCN(c2ccc(OS(=O)(=O)F)cc2)CC1] |
| 62 | MFS-8-3 | 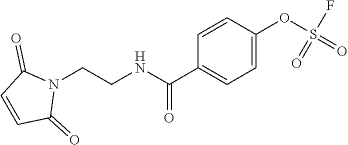 [SMILES: O=C(NCCN1C(=O)C=CC1=O)c1ccc(OS(=O)(=O)F)cc1] |
| 63 | MFS-9-4 | 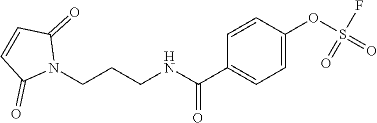 [SMILES: O=C(NCCCN1C(=O)C=CC1=O)c1ccc(OS(=O)(=O)F)cc1] |
| 64 | PFS-5-1 | 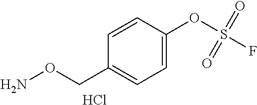 [SMILES: NOCc1ccc(OS(=O)(=O)F)cc1 ; Cl] |
| 65 | PFS-6-1 | 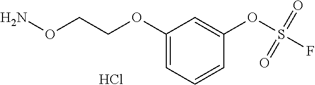 [SMILES: NOCCOc1cccc(OS(=O)(=O)F)c1 ; Cl] |
| 66 | PFS-7-1 | 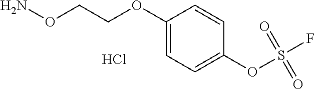 [SMILES: NOCCOc1ccc(OS(=O)(=O)F)cc1 ; Cl] |
| 67 | MFS-5-2 | 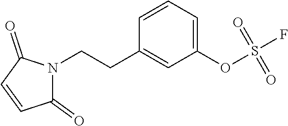 [SMILES: O=C1C=CC(=O)N1CCc1cccc(OS(=O)(=O)F)c1] |
| 68 | MFS-10-1 | 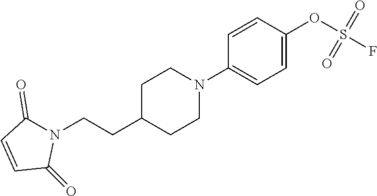 [SMILES: O=C1C=CC(=O)N1CCC1CCN(c2ccc(OS(=O)(=O)F)cc2)CC1] |
| 69 | MFS-10-2 | 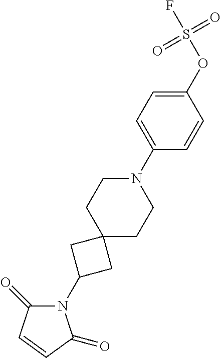 [SMILES: O=C1C=CC(=O)N1C1CC2(CCN(c3ccc(OS(=O)(=O)F)cc3)CC2)C1] |
| 70 | MFS-10-3 | 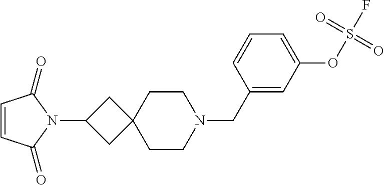 [SMILES: O=C1C=CC(=O)N1C1CC2(CCN(Cc3cccc(OS(=O)(=O)F)c3)CC2)C1] |
| 71 | MFS-11-2 | 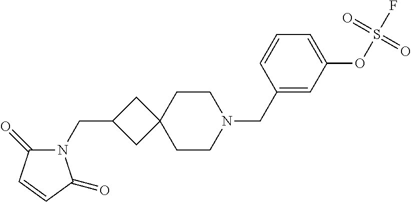 [SMILES: O=C1C=CC(=O)N1CC1CC2(CCN(Cc3cccc(OS(=O)(=O)F)c3)CC2)C1] |
| 72 | MFS-11-5 | 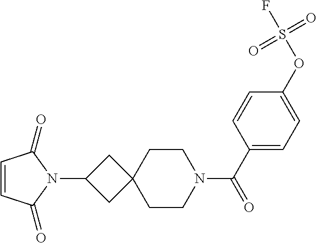 [SMILES: O=C(c1ccc(OS(=O)(=O)F)cc1)N1CCC2(CC1)CC(N1C(=O)C=CC1=O)C2] |
| 73 | MFS-11-6 | 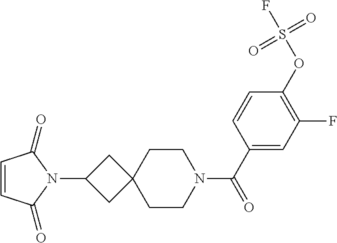 [SMILES: O=C(c1ccc(OS(=O)(=O)F)c(F)c1)N1CCC2(CC1)CC(N1C(=O)C=CC1=O)C2] |
| 74 | MFS-12-1 | 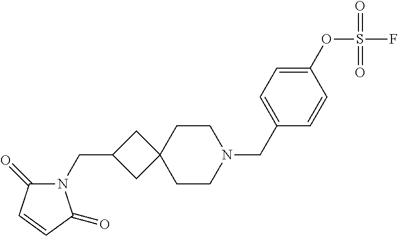 [SMILES: O=C1C=CC(=O)N1CC1CC2(CCN(Cc3ccc(OS(=O)(=O)F)cc3)CC2)C1] |
| 75 | SFY-4-1 | 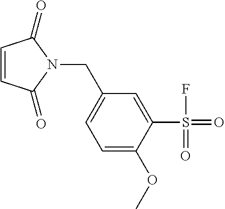 [SMILES: COc1ccc(CN2C(=O)C=CC2=O)cc1S(=O)(=O)F] |
| 78 | MFS-5-11 | 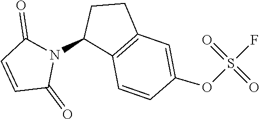 [SMILES: O=C1C=CC(=O)N1[C@H]1CCc2cc(OS(=O)(=O)F)ccc21] |
| 82 | MFS-8-7 | 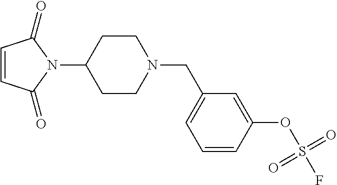 [SMILES: O=C1C=CC(=O)N1C1CCN(Cc2cccc(OS(=O)(=O)F)c2)CC1] |
| 84 | MFS-10-9 | 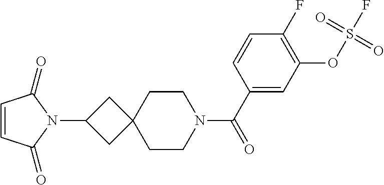 [SMILES: O=C(c1ccc(F)c(OS(=O)(=O)F)c1)N1CCC2(CC1)CC(N1C(=O)C=CC1=O)C2] |
| 87 | MFS-3-8 | 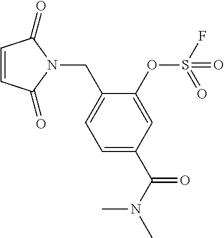 [SMILES: CN(C)C(=O)c1ccc(CN2C(=O)C=CC2=O)c(OS(=O)(=O)F)c1] |
| 88 | MFS-3-16 | 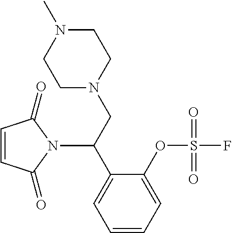 [SMILES: CN1CCN(CC(c2ccccc2OS(=O)(=O)F)N2C(=O)C=CC2=O)CC1] |
| 89 | MFS-4-1 | 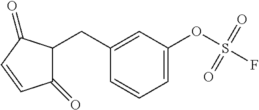 [SMILES: O=C1C=CC(=O)C1Cc1cccc(OS(=O)(=O)F)c1] |
| 90 | MFS-4-12 | 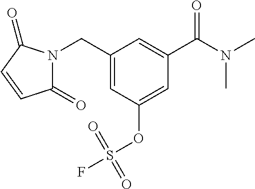 [SMILES: CN(C)C(=O)c1cc(CN2C(=O)C=CC2=O)cc(OS(=O)(=O)F)c1] |
| 91 | MFS-5-1 | 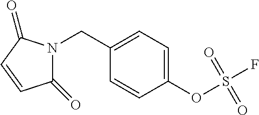 [SMILES: O=C1C=CC(=O)N1Cc1ccc(OS(=O)(=O)F)cc1] |
| 92 | MFS-5-3 | 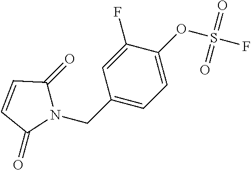 [SMILES: O=C1C=CC(=O)N1Cc1ccc(OS(=O)(=O)F)c(F)c1] |
| 93 | MFS-5-4 | 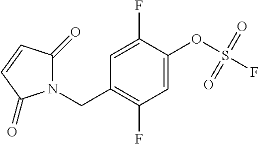 [SMILES: O=C1C=CC(=O)N1Cc1cc(F)c(OS(=O)(=O)F)cc1F] |
| 94 | MFS-5-7 | 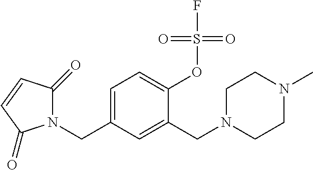 [SMILES: CN1CCN(Cc2cc(CN3C(=O)C=CC3=O)ccc2OS(=O)(=O)F)CC1] |
| 95 | MFS-5-8 | 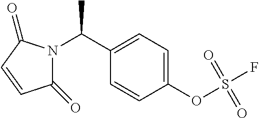 [SMILES: C[C@@H](c1ccc(OS(=O)(=O)F)cc1)N1C(=O)C=CC1=O] |
| 96 | MFS-5-9 | 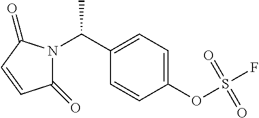 [SMILES: C[C@H](c1ccc(OS(=O)(=O)F)cc1)N1C(=O)C=CC1=O] |
| 97 | MFS-5-10 | 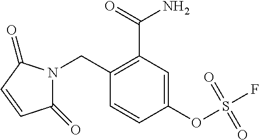 [SMILES: NC(=O)c1cc(OS(=O)(=O)F)ccc1CN1C(=O)C=CC1=O] |
| 98 | MFS-5-13 | 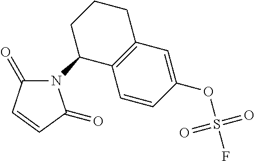 [SMILES: O=C1C=CC(=O)N1[C@H]1CCCc2cc(OS(=O)(=O)F)ccc21] |
| 99 | MFS-5-14 | 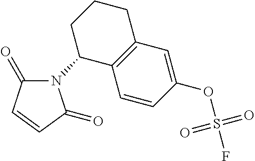 [SMILES: O=C1C=CC(=O)N1[C@@H]1CCCc2cc(OS(=O)(=O)F)ccc21] |
| 100 | MFS-8-1 | 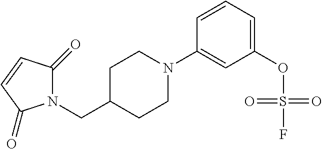 [SMILES: O=C1C=CC(=O)N1CC1CCN(c2cccc(OS(=O)(=O)F)c2)CC1] |
| 101 | | 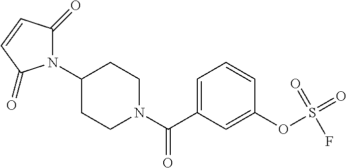 [SMILES: O=C(c1cccc(OS(=O)(=O)F)c1)N1CCC(N2C(=O)C=CC2=O)CC1] |
| 102 | MFS-8-5 | 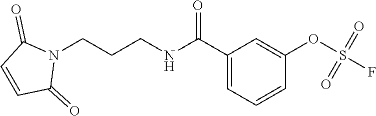 [SMILES: O=C(NCCCN1C(=O)C=CC1=O)c1cccc(OS(=O)(=O)F)c1] |
| 103 | MFS-9-1 | 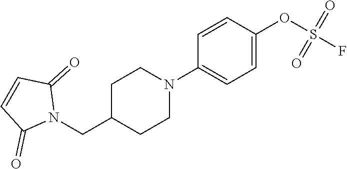 [SMILES: O=C1C=CC(=O)N1CC1CCN(c2ccc(OS(=O)(=O)F)cc2)CC1] |
| 104 | MFS-9-5 | 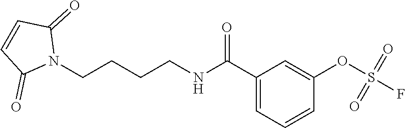 [SMILES: O=C(NCCCCN1C(=O)C=CC1=O)c1cccc(OS(=O)(=O)F)c1] |
| 105 | MFS-9-7 | 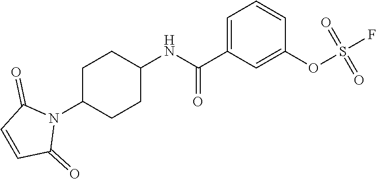 [SMILES: O=C(NC1CCC(N2C(=O)C=CC2=O)CC1)c1cccc(OS(=O)(=O)F)c1] |
| 106 | MFS-9-2 | 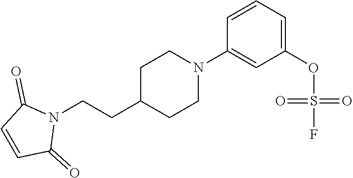 [SMILES: O=C1C=CC(=O)N1CCC1CCN(c2cccc(OS(=O)(=O)F)c2)CC1] |
| 107 | MFS-10-5 | 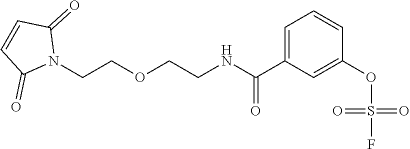 [SMILES: O=C(NCCOCCN1C(=O)C=CC1=O)c1cccc(OS(=O)(=O)F)c1] |
| 108 | MFS-11-4 | 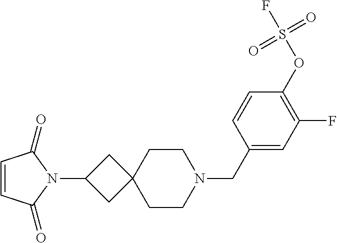 [SMILES: O=C1C=CC(=O)N1C1CC2(CCN(Cc3ccc(OS(=O)(=O)F)c(F)c3)CC2)C1] |
| 110 | FP-FS-2 | 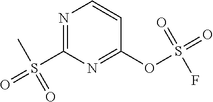 |
| 111 | MFS-10-4 | 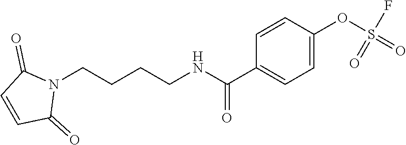 [SMILES: O=C(NCCCCN1C(=O)C=CC1=O)c1ccc(OS(=O)(=O)F)cc1] |
| 112 | MFS-10-6 | 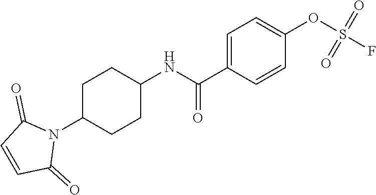 [SMILES: O=C(NC1CCC(N2C(=O)C=CC2=O)CC1)c1ccc(OS(=O)(=O)F)cc1] |
| 113 | MFS-10-7 | 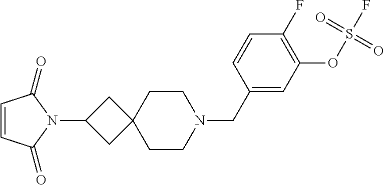 [SMILES: O=C1C=CC(=O)N1C1CC2(CCN(Cc3ccc(F)c(OS(=O)(=O)F)c3)CC2)C1] |
| 114 | MFS-10-8 | 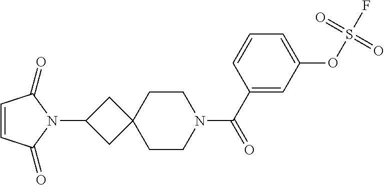 [SMILES: O=C(c1cccc(OS(=O)(=O)F)c1)N1CCC2(CC1)CC(N1C(=O)C=CC1=O)C2] |
| 115 | MFS-11-1 | 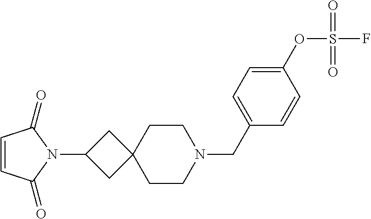 [SMILES: O=C1C=CC(=O)N1C1CC2(CCN(Cc3ccc(OS(=O)(=O)F)cc3)CC2)C1] |
| 116 | MFS-11-3 | 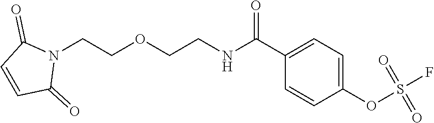 [SMILES: O=C(NCCOCCN1C(=O)C=CC1=O)c1ccc(OS(=O)(=O)F)cc1] |
| 117 | BAFS-3-1 | 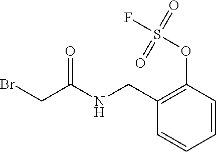 [SMILES: O=C(CBr)NCc1ccccc1OS(=O)(=O)F] |
| 118 | BAFS-3-2 | 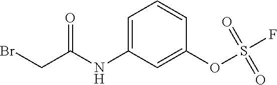 [SMILES: O=C(CBr)Nc1cccc(OS(=O)(=O)F)c1] |
| 119 | BAFS-3-3 | 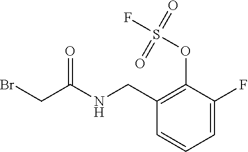 [SMILES: O=C(CBr)NCc1cccc(F)c1OS(=O)(=O)F] |
| 120 | BAFS-3-4 | 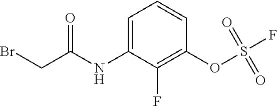 [SMILES: O=C(CBr)Nc1cccc(OS(=O)(=O)F)c1F] |
| 121 | BAFS-4-1 | 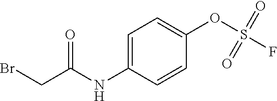 [SMILES: O=C(CBr)Nc1ccc(OS(=O)(=O)F)cc1] |
| 122 | BAFS-4-2 | 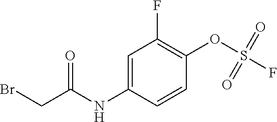 [SMILES: O=C(CBr)Nc1ccc(OS(=O)(=O)F)c(F)c1] |
| 123 | BAFS-4-3 | 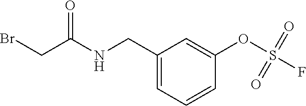 [SMILES: O=C(CBr)NCc1cccc(OS(=O)(=O)F)c1] |
| 124 | BAFS-4-4 | 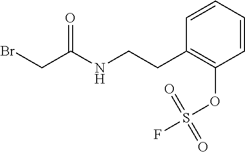 [SMILES: O=C(CBr)NCCc1ccccc1OS(=O)(=O)F] |
| 125 | BAFS-5-1 | 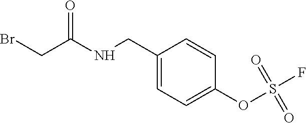 [SMILES: O=C(CBr)NCc1ccc(OS(=O)(=O)F)cc1] |
| 126 | BFS-2-1 | 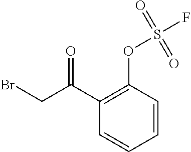 [SMILES: O=C(CBr)c1ccccc1OS(=O)(=O)F] |
| 127 | BFS-2-2 | 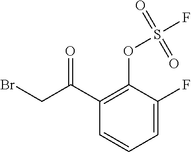 [SMILES: O=C(CBr)c1cccc(F)c1OS(=O)(=O)F] |
| 128 | BFS-3-1 | 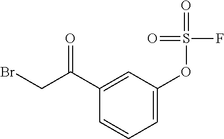 [SMILES: O=C(CBr)c1cccc(OS(=O)(=O)F)c1] |
| 129 | BFS-3-3 | 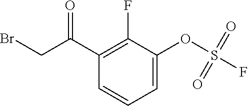 [SMILES: O=C(CBr)c1cccc(OS(=O)(=O)F)c1F] |
| 130 | BFS-4-1 | 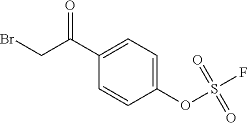 [SMILES: O=C(CBr)c1ccc(OS(=O)(=O)F)cc1] |
| 131 | BFS-4-2 | 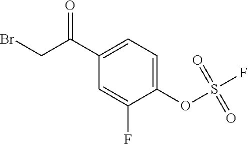 [SMILES: O=C(CBr)c1ccc(OS(=O)(=O)F)c(F)c1] |
| 133 | BFS-3-2 | 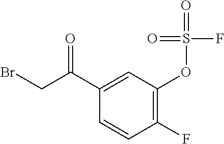 [SMILES: O=C(CBr)c1ccc(F)c(OS(=O)(=O)F)c1] |
| 134 | AFS-3-2 | 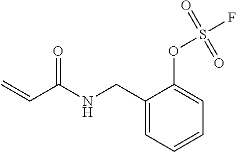 [SMILES: C=CC(=O)NCc1ccccc1OS(=O)(=O)F] |
| 135 | PFS-3-2 | 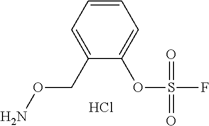 [SMILES: NOCc1ccccc1OS(=O)(=O)F ; Cl] |
| 136 | PFS-4-2 | 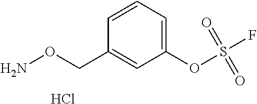 [SMILES: NOCc1cccc(OS(=O)(=O)F)c1 ; Cl] |
| 138 | PFS-6-2 | 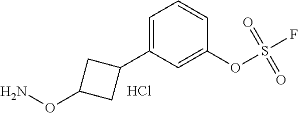 [SMILES: NOC1CC(c2cccc(OS(=O)(=O)F)c2)C1 ; Cl] |
| 140 | PFS-7-2 | 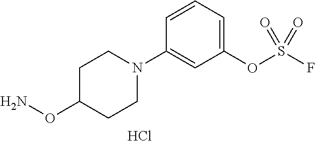 [SMILES: NOC1CCN(c2cccc(OS(=O)(=O)F)c2)CC1 ; Cl] |
| 141 | PFS-7-3 | 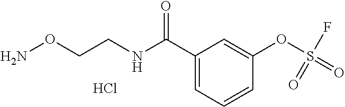 [SMILES: NOCCNC(=O)c1cccc(OS(=O)(=O)F)c1 ; Cl] |
| 142 | PFS-8-1 | 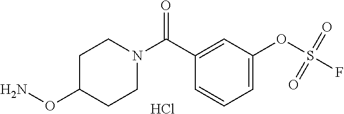 [SMILES: NOC1CCN(C(=O)c2cccc(OS(=O)(=O)F)c2)CC1 ; Cl] |
| 144 | PFS-8-3 | 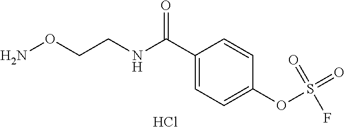 [SMILES: NOCCNC(=O)c1ccc(OS(=O)(=O)F)cc1 ; Cl] |
| 145 | FSK-02 | 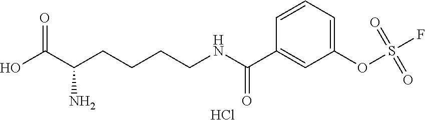 [SMILES: N[C@@H](CCCCNC(=O)c1cccc(OS(=O)(=O)F)c1)C(=O)O ; Cl] |
| 146 | SP-FS-1 | 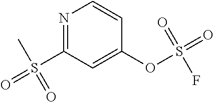 |
| 147 | SP-FS-2 | 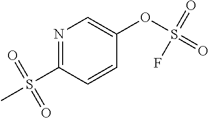 |
| 148 | SP-FS-3 | 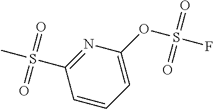 |
| 149 | CAFS-3-A1 | 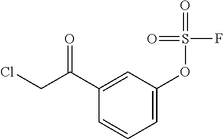 [SMILES: O=C(CCl)c1cccc(OS(=O)(=O)F)c1] |
| 150 | FP-FS-1 | 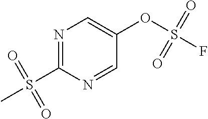 |
| 151 | FSK-01 | 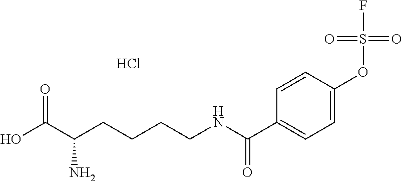 [SMILES: N[C@@H](CCCCNC(=O)c1ccc(OS(=O)(=O)F)cc1)C(=O)O ; Cl] |

### Proteins

Some embodiments provide a protein comprising an antigen-binding domain, wherein the antigen-binding domain comprising an oxime, the oxime having the structure:

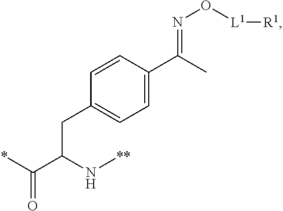

wherein:
* and ** represent the points of connection of the oxime to the antigen-binding domain;
L¹ is a bond or a C1-C6 alkylene optionally substituted with 1-2 substituents independently selected from halogen and C3-C6 cycloalkyl, and wherein 1-2 alkylene units are optionally replaced by O, N, C3-C6 cycloalkyl, or phenyl;
R¹ is azido, tetrazinyl, a C2-C3 alkyne, or an optionally substituted C8-C12 cycloalkyne.

In some embodiments, the oxime is connected to the antigen-binding domain via an L amino acid. In some embodiments, the oxime is connected to the antigen-binding domain via a D amino acid.

In some embodiments, L¹ is a C1-C6 alkylene optionally substituted with 1-2 substituents independently selected from halogen and C3-C6 cycloalkyl, and wherein 1-2 alkylene units are optionally replaced by O, N, C3-C6 cycloalkyl, or phenyl.

In some embodiments, L¹ is a C1-C6 alkylene substituted with 1-2 substituents independently selected from halogen and C3-C6 cycloalkyl.

In some embodiments, L¹ is a C1-C6 alkylene wherein 1-2 alkylene units are replaced by O, N, C3-C6 cycloalkyl, or phenyl.

In some embodiments, L¹ is a C1-C6 alkylene. In some embodiments, L¹ is methylene or ethylene. In some embodiments, L¹ is n-propylene or isopropylene.

In some embodiments, L¹ is a C4-C6 cycloalkyl (for example, by replacing a single methylene group with the C4-C6 cycloalkyl).

In some embodiments, L¹ is a PEG unit.

In some embodiments, L¹ is

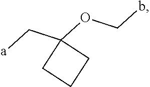

wherein “a” represents the point of connection of L¹ to the oxime and “b” represents the point of connection of L¹ to R¹.

In some embodiments, L¹ is a bond.

In some embodiments, R¹ is azido.

In some embodiments, R¹ is tetrazinyl.

In some embodiments, R¹ is a C2-C3 alkyne.

In some embodiments, R¹ is an optionally substituted C8-C12 cycloalkyne. In some embodiments, R¹ is a C8-C12 cycloalkyne. In some embodiments, R¹ is

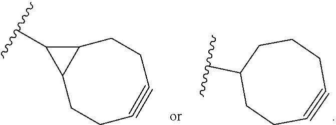 [SMILES: *C1C2CCC#CCCC12 ; *C1CCC#CCCC1]

In some embodiments, R¹ is

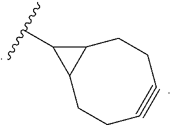 [SMILES: "." ; *C1C2CCC#CCCC12]

In some embodiments, R¹ is

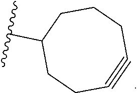 [SMILES: *C1CCC#CCCC1]

Some embodiments provide a protein comprising an antigen-binding domain, wherein the antigen-binding domain comprises a modified phenylalanine residue, the modified phenylalanine residue having the structure:

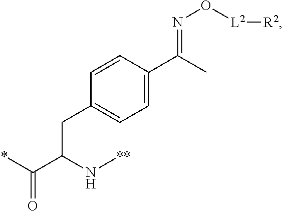

wherein:
* and ** represent the points of connection of the modified phenylalanine residue to the antigen-binding domain;
L² is a C2-C16 alkylene optionally substituted with 1-2 substituents independently selected from halogen and C3-C6 cycloalkyl, and wherein 1-6 alkylene units are optionally replaced by O, N, S, C3-C10 cycloalkyl, phenyl, 5-12 membered heteroaryl, and 4-14 membered heterocyclyl;
R² is

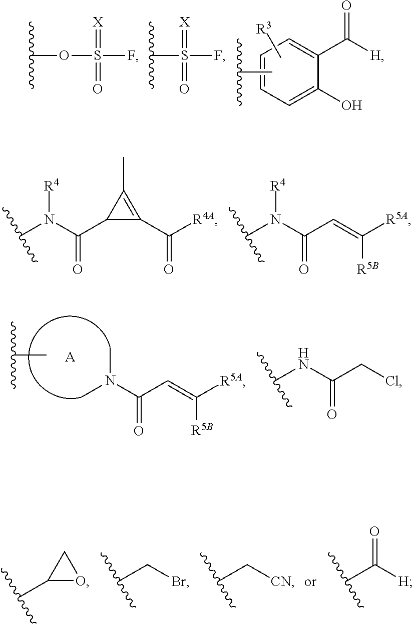

X is O or NR^{X};
R^{X} is hydrogen, C1-C6 alkyl, or C3-C6 cycloalkyl;
R³ is halogen or C1-C6 alkyl;
R⁴ is hydrogen or C1-C6 alkyl;
R^{4A} is C1-C6 alkyl or C3-C6 cycloalkyl;
R^{5A} and R^{5B} are independently hydrogen, halogen, or C1-C6 alkyl; or
R^{5A} and R^{5B} together with the carbon atom to which they are attached form a cyclopropyl;
Ring A is a 4-10 membered heterocyclyl.

In some embodiments, L² is a C2-C16 alkylene optionally substituted with 1-2 substituents independently selected from halogen and C3-C6 cycloalkyl, and wherein 1-6 alkylene units are optionally replaced by O, N, S, C3-C10 cycloalkyl, phenyl, 5-12 membered heteroaryl, —C(O)—, and 4-14 membered heterocyclyl.

In some embodiments, L² is a C1-C16 alkylene optionally substituted with 1-2 substituents independently selected from halogen and C3-C6 cycloalkyl, and wherein 1-6 alkylene units are optionally replaced by O, N, S, C3-C10 cycloalkyl, phenyl, 5-12 membered heteroaryl, —C(O)—, and 4-14 membered heterocyclyl.

Some embodiments provide a protein comprising an antigen-binding domain, wherein the antigen-binding domain comprises a modified cysteine residue, the modified cysteine residue having the structure:

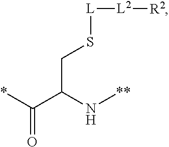

wherein:
* and ** represent the points of connection of the modified cysteine residue to the antigen-binding domain;
L is a bond,

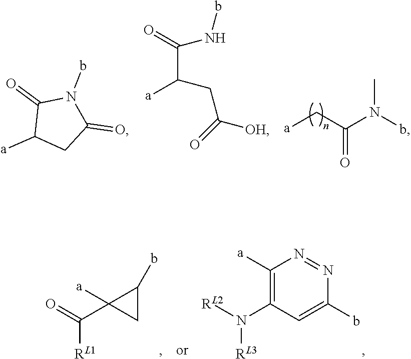

wherein “a” represents the point of connection of L to the sulfur atom of the modified cysteine residue and “b” represents the point of connection of L to L²;
n is 1 or 2;
R^{L1}, R^{L2}, and R^{L3}, are each independently selected C1-C10 alkyl;
L² is a C2-C16 alkylene optionally substituted with 1-2 substituents independently selected from halogen and C3-C6 cycloalkyl, and wherein 1-6 alkylene units are optionally replaced by O, N, S, C3-C10 cycloalkyl, phenyl, 5-12 membered heteroaryl, and 4-14 membered heterocyclyl;
R² is

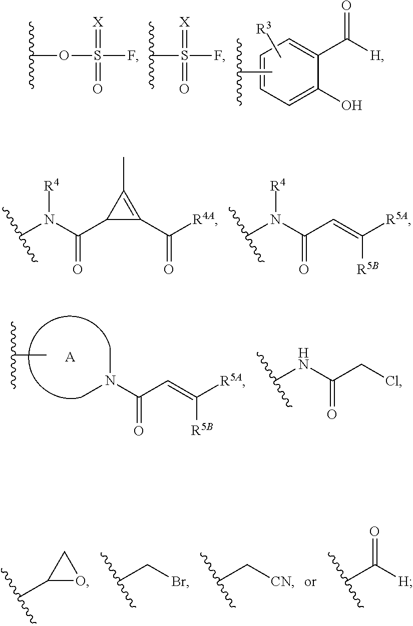

X is O or NR^{X};
R^{X} is hydrogen, C1-C6 alkyl, or C3-C6 cycloalkyl;
R³ is halogen or C1-C6 alkyl;
R⁴ is hydrogen or C1-C6 alkyl;
R^{4A} is C1-C6 alkyl or C3-C6 cycloalkyl;
R^{5A} and R^{5B} are independently hydrogen, halogen, or C1-C6 alkyl; or
R^{5A} and R^{5B} together with the carbon atom to which they are attached form a cyclopropyl;
Ring A is a 4-10 membered heterocyclyl.

In some embodiments, L² is a C2-C16 alkylene optionally substituted with 1-2 substituents independently selected from halogen and C3-C6 cycloalkyl, and wherein 1-6 alkylene units are optionally replaced by O, N, S, C3-C10 cycloalkyl, phenyl, 5-12 membered heteroaryl, —C(O)—, and 4-14 membered heterocyclyl.

In some embodiments, L² is a C1-C16 alkylene optionally substituted with 1-2 substituents independently selected from halogen and C3-C6 cycloalkyl, and wherein 1-6 alkylene units are optionally replaced by O, N, S, C3-C10 cycloalkyl, phenyl, 5-12 membered heteroaryl, —C(O)—, and 4-14 membered heterocyclyl.

In some embodiments, it is provided that the structure does not contain a GLP-1 peptide or variant thereof, optionally wherein the structure does not contain a modified GLP-1 peptide, optionally wherein the structure does not contain a peptide as described in WO 2025/076010.

In some embodiments, L is

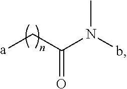

wherein “a” represents the point of connection of L to the sulfur atom of the modified cysteine residue and “b” represents the point of connection of L to L².

In some embodiments, n is 1. In some embodiments, n is 2.

In some embodiments, L is

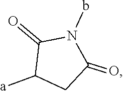

wherein “a” represents the point of connection of L to the sulfur atom of the modified cysteine residue and “b” represents the point of connection of L to L².

In some embodiments, L is

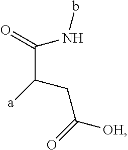

wherein “a” represents the point of connection of L to the sulfur atom of the modified cysteine residue and “b” represents the point of connection of L to L².

In some embodiments, L is

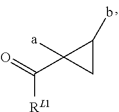

wherein “a” represents the point of connection of L to the sulfur atom of the modified cysteine residue and “b” represents the point of connection of L to L².

In some embodiments, R^{L1} is a C1-C6 alkyl. In some embodiments, R^{L1} is ethyl.

In some embodiments, L is

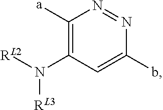

wherein “a” represents the point of connection of L to the sulfur atom of the modified cysteine residue and “b” represents the point of connection of L to L².

In some embodiments, R^{L2} and R^{L3} are independently selected C1-C6 alkyl. In some embodiments, R^{L2} and R^{L3} are each methyl.

In some embodiments, L is a bond.

In some embodiments, L² is a C2-C16 alkylene optionally substituted with 1-2 substituents independently selected from halogen and C3-C6 cycloalkyl.

In some embodiments, L² is a C2-C16 alkylene wherein 1-6 alkylene units are optionally replaced by O, N, S, C3-C10 cycloalkyl, phenyl, 5-12 membered heteroaryl, and 4-14 membered heterocyclyl.

In some embodiments, L² is a C2-C16 alkylene substituted with 1-2 substituents independently selected from halogen and C3-C6 cycloalkyl, and wherein 1-6 alkylene units are optionally replaced by O, N, S, C3-C10 cycloalkyl, phenyl, 5-12 membered heteroaryl, and 4-14 membered heterocyclyl.

In some embodiments, L² is a C2-C16 alkylene optionally substituted with 1-2 substituents independently selected from halogen and C3-C6 cycloalkyl, and wherein 1-6 alkylene units are replaced by O, N, S, C3-C10 cycloalkyl, phenyl, 5-12 membered heteroaryl, and 4-14 membered heterocyclyl.

In some embodiments, L² is a C2-C16 alkylene substituted with 1-2 substituents independently selected from halogen and C3-C6 cycloalkyl, and wherein 1-6 alkylene units are replaced by O, N, S, C3-C10 cycloalkyl, phenyl, 5-12 membered heteroaryl, and 4-14 membered heterocyclyl.

In some embodiments, L² is a C2-C16 alkylene. In some embodiments, L² is a C2-C6 alkylene.

In some embodiments, L² comprises one triazole ring. In some embodiments, the triazole is a single 5-membered ring. In some embodiments, the triazole is part of a larger (e.g., fused) ring system.

In some embodiments, L² is selected from the group consisting of:

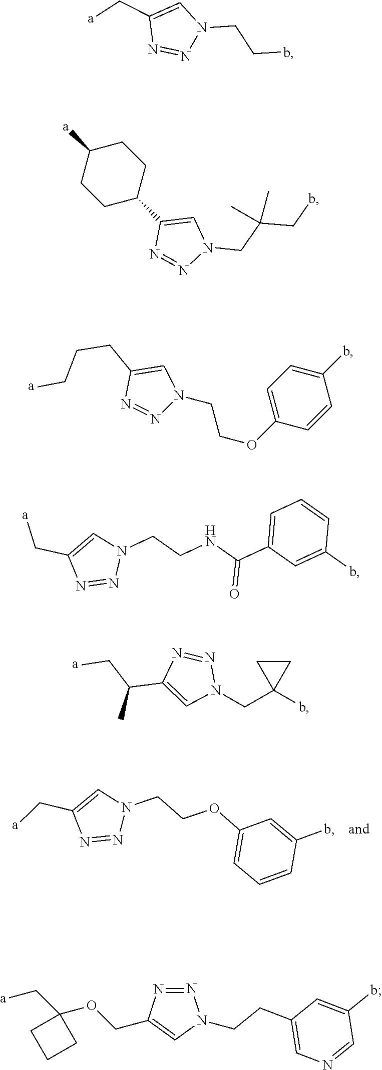

wherein “a” represents the point of connection of L² to L or to the modified phenylalanine residue or to the modified cysteine residue and “b” represents the point of connection of L² to R².

In some embodiments, L² is selected from the group consisting of:

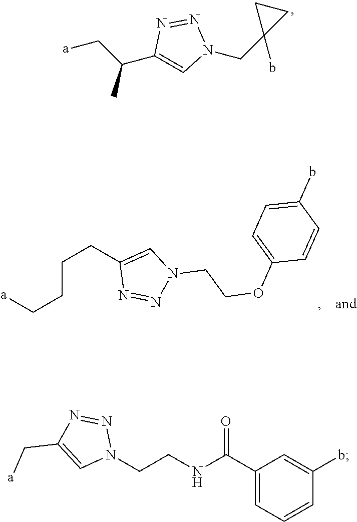

wherein “a” represents the point of connection of L² to L or to the modified phenylalanine residue or to the modified cysteine residue and “b” represents the point of connection of L² to R².

In some embodiments, L² is selected from the group consisting of:

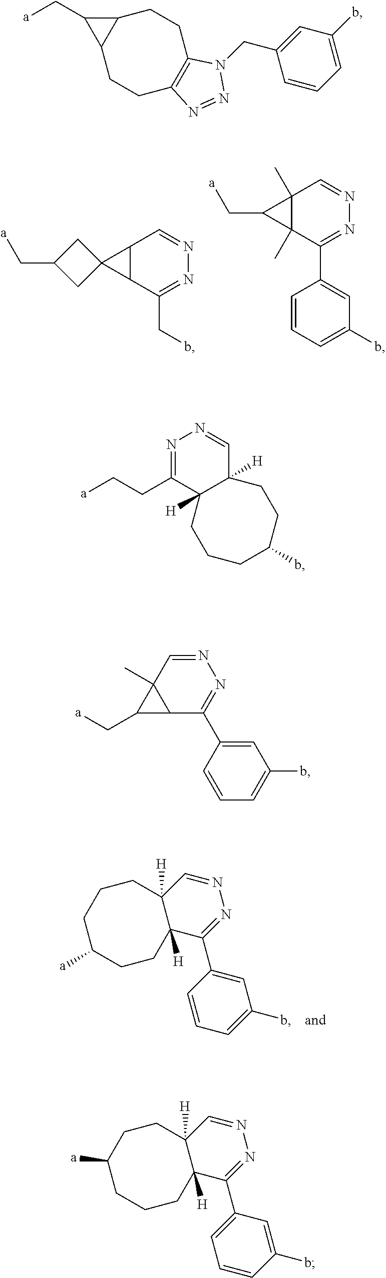

wherein “a” represents the point of connection of L² to L or to the modified phenylalanine residue or to the modified cysteine residue and “b” represents the point of connection of L² to R².

In some embodiments, L² is

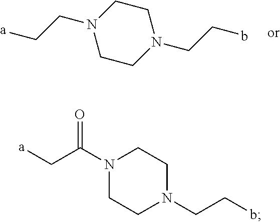

wherein “a” represents the point of connection of L² to L or to the modified phenylalanine residue or to the modified cysteine residue and “b” represents the point of connection of L² to R².

In some embodiments, R² is

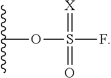

In some embodiments, X is NR^{X}.

In some embodiments, R^{X} is hydrogen.

In some embodiments, R^{X} is C1-C6 alkyl. In some embodiments, R^{X} is methyl.

In some embodiments, R^{X} is C3-C6 cycloalkyl. In some embodiments, R^{X} is cyclopropyl.

In some embodiments, X is O.

In some embodiments, R² is

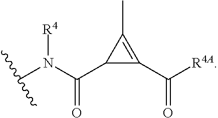

In some embodiments, R^{4A} is C1-C6 alkyl. In some embodiments, R^{4A} is methyl.

In some embodiments, R^{4A} is C3-C6 cycloalkyl.

In some embodiments, R² is

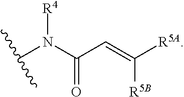

In some embodiments, R⁴ is C1-C6 alkyl. In some embodiments, R⁴ is methyl.

In some embodiments, R⁴ is hydrogen.

In some embodiments, R² is

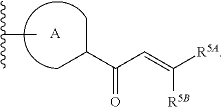

In some embodiments, R^{5A} and R^{5B} are independently hydrogen, halogen, or C1-C6 alkyl.

In some embodiments, R^{5A} and R^{5B} are each hydrogen. In some embodiments, R^{5A} and R^{5B} are each independently C1-C6 alkyl. In some embodiments, one of R^{5A} and R^{5B} is hydrogen and the other of R^{5A} and R^{5B} is C1-C6 alkyl. In some embodiments, one of R^{5A} and R^{5B} is halogen and the other of R^{5A} and R^{5B} is hydrogen, halogen, or C1-C6 alkyl.

In some embodiments, R^{5A} and R^{5B} together with the carbon atom to which they are attached form a cyclopropyl.

In some embodiments, Ring A is a 4-10 membered heterocyclyl. In some embodiments, Ring A is a 5-6 membered heterocyclyl. In some embodiments, Ring A is piperidine or piperazine.

In some embodiments, R² is

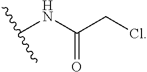 [SMILES: *NC(=O)CCl]

In some embodiments, R² is

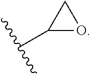 [SMILES: *C1CO1]

In some embodiments, R² is

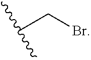 [SMILES: *CBr]

In some embodiments, R² is

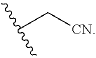 [SMILES: *CC#N]

In some embodiments, R² is

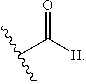

In some embodiments, L² is selected from the group consisting of:

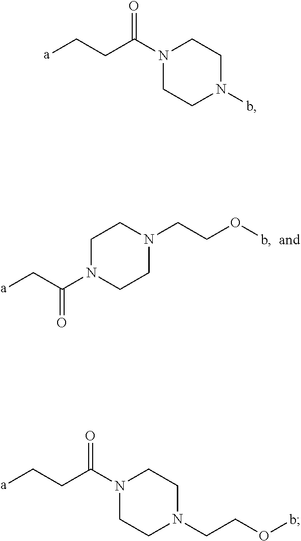

wherein “a” represents the point of connection of L² to L or to the modified phenylalanine residue or to the modified cysteine residue and “b” represents the point of connection of L² to R².

In some embodiments, R² is

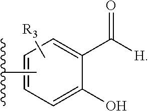

In some embodiments, R³ is halogen.

In some embodiments, R³ is C1-C6 alkyl.

In some embodiments, -L²-R² is

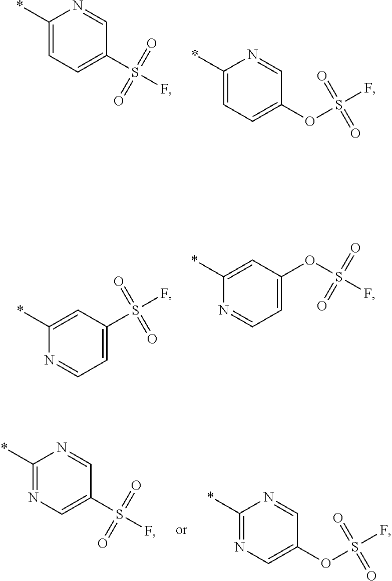

wherein * represents the point of connection of L² to L.

In some embodiments, the modified amino acid residue is an L modified amino acid residue. In some embodiments, the modified amino acid residue is a D modified amino acid residue.

In some embodiments, the modified amino acid residue is present in a CDR of the antigen-binding domain. In some embodiments, the CDR is a heavy chain CDR. In some embodiments, the CDR is a light chain CDR.

In some embodiments, the modified amino acid residue is present in a framework region of the antigen-binding domain.

In some embodiments, the modified phenylalanine residue is an L modified phenylalanine residue. In some embodiments, the modified phenylalanine residue is a D modified phenylalanine residue.

In some embodiments, the modified phenylalanine residue is present in a CDR of the antigen-binding domain. In some embodiments, the CDR is a heavy chain CDR. In some embodiments, the CDR is a light chain CDR.

In some embodiments, the modified phenylalanine residue is present in a framework region of the antigen-binding domain.

In some embodiments, the modified cysteine residue is an L modified cysteine residue. In some embodiments, the modified cysteine residue is a D modified cysteine residue.

In some embodiments, the modified cysteine residue is present in a CDR of the antigen-binding domain. In some embodiments, the CDR is a heavy chain CDR. In some embodiments, the CDR is a light chain CDR.

In some embodiments, the modified cysteine residue is present in a framework region of the antigen-binding domain.

In some embodiments, the protein is an antibody. In some embodiments, the antibody is a human antibody, a humanized antibody, or a veneered antibody. In some embodiments, the antibody is a human IgG1, human IgG2, human IgG3, or human IgG4 antibody.

In some embodiments, the protein is or comprises a single chain Fv (scFv), a VHH, a VNAR, a DARpin, a single domain antibody (sdAb), an Adnectin/Centyrin, an Affibody, a Knottin, a bicyclic peptide, or a cyclic peptide.

In some embodiments, the protein further comprises a conjugated cytotoxic or cytostatic agent.

In some embodiments, the protein comprises a radioisotope. Examples of radioisotopes 10 include, but are not limited to At²¹¹, I¹³¹, I¹²⁵, Y⁹⁰, Re¹⁸⁶, Re¹⁸⁸, Sm¹⁵³, Bi²¹² or²¹³, P³² and radioactive isotopes of Lu including Lu¹⁷⁷.

In some embodiments, the antigen-binding domain specifically binds to a target protein.

In some embodiments, the target protein comprises an extracellular domain, and the antigen-binding domain specifically binds to the extracellular domain.

Any of the proteins comprising an antigen-binding domain described herein can be a single polypeptide, or can include two, three, four, five, six, seven, eight, nine, or ten (the same or different) polypeptides. In some embodiments where the protein is a single polypeptide, the protein can include a single antigen-binding domain or two antigen-binding domains. In some embodiments where the protein is a single polypeptide and includes two antigen-binding domains, the first and second antigen-binding domains can be identical or different from each other (and can specifically bind to the same or different antigens or epitopes).

In some embodiments where the protein is a single polypeptide, the antigen-binding domain can each be independently selected from the group of: a VH domain, a VHH domain, a VNAR domain, a scFv, a DARpin, a single domain antibody (sdAb), an Adnectin/Centyrin, an Affibody, a bicyclic peptide (see, e.g., Eder et al., Cancer Res. 79(4):841-852, 2019; Gan et al., J. Med. Chem. 66(21):14623-14632, 2023), or a cyclic peptide (see, e.g., Costa et al., Pharmaceuticals 16(7):996, 2023). In some embodiments where the protein is a single polypeptide, the protein can comprise or be a BiTe, a (scFv)₂, a nanobody, a nanobody-HSA, a DART, a TandAb, a scDiabody, a scDiabody-CH3, scFv-CH-CL-scFv, a HSAbody, scDiabody-HAS, a tandem-scFv, a scFv, a sdAb, an Affibody, a Knottin, an Adnectin/Centyrin, a DARPin, a fibronectin, a DEP conjugate, a bicyclic peptide, or a cyclic peptide. Additional examples of antigen-binding domains that can be used when the protein is a single polypeptide are known in the art.

A V_{H}H domain is a single monomeric variable antibody domain that can be found in camelids. A V_{NAR} domain is a single monomeric variable antibody domain that can be found in cartilaginous fish. Non-limiting aspects of V_{H}H domains and V_{NAR} domains are described in, e.g., Cromie et al., Curr. Top. Med. Chem. 15:2543-2557, 2016; De Genst et al., Dev. Comp. Immunol. 30:187-198, 2006; De Meyer et al., Trends Biotechnol. 32:263-270, 2014; Kijanka et al., Nanomedicine 10:161-174, 2015; Kovaleva et al., Expert. Opin. Biol. Ther. 14:1527-1539, 2014; Krah et al., Immunopharmacol. Immunotoxicol. 38:21-28, 2016; Mujic-Delic et al., Trends Pharmacol. Sci. 35:247-255, 2014; Muyldermans, J. Biotechnol. 74:277-302, 2001; Muyldermans et al., Trends Biochem. Sci. 26:230-235, 2001; Muyldermans, Ann. Rev. Biochem. 82:775-797, 2013; Rahbarizadeh et al., Immunol. Invest. 40:299-338, 2011; Van Audenhove et al., EBioMedicine 8:40-48, 2016; Van Bockstaele et al., Curr. Opin. Investig. Drugs 10:1212-1224, 2009; Vincke et al., Methods Mol. Biol. 911:15-26, 2012; and Wesolowski et al., Med. Microbiol. Immunol. 198:157-174, 2009.

In some embodiments where the protein is a single polypeptide and includes two antigen-binding domains, the first antigen-binding domain and the second antigen-binding domain can both be VHH domains, or at least one antigen-binding domain can be a VHH domain. In some embodiments where the protein is a single polypeptide and includes two antigen-binding domains, the first antigen-binding domain and the second antigen-binding domain are both V_{NAR} domains, or at least one antigen-binding domain is a V_{NAR} domain. In some embodiments where the protein is a single polypeptide, the antigen-binding domain is a scFv domain. In some embodiments where the protein is a single polypeptide and includes two antigen-binding domains, the first antigen-binding domain and the second antigen-binding domain can both be scFv domains, or at least one antigen-binding domain can be a scFv domain.

In some embodiments, the protein can include two or more polypeptides (e.g., two, three, four, five, six, seven, eight, nine, or ten polypeptides). In some embodiments where the protein includes two or more polypeptides, two, three, four, five or six of the polypeptides of the two or more polypeptides can be identical.

In some embodiments where the protein includes two or more polypeptides (e.g., two, three, four, five, six, seven, eight, nine, or ten polypeptides), two or more of the polypeptides of the protein can assemble (e.g., non-covalently assemble) to form one or more antigen-binding domains, e.g., an antigen-binding fragment of an antibody (e.g., any of the antigen-binding fragments of an antibody described herein), a VHH-scAb, a VHH-Fab, a Dual scFab, a F(ab′)2, a diabody, a crossMab, a DAF (two-in-one), a DAF (four-in-one), a DutaMab, a DT-IgG, a knobs-in-holes common light chain, a knobs-in-holes assembly, a charge pair, a Fab-arm exchange, a SEEDbody, a LUZ-Y, a Fcab, a Kk-body, an orthogonal Fab, a DVD-IgG, a IgG(H)-scFv, a scFv-(H)IgG, IgG(L)-scFv, scFv-(L)IgG, IgG(L,H)-Fv, IgG(H)—V, V(H)—IgG, IgG(L)-V, V(L)-IgG, KIH IgG-scFab, 2scFv-IgG, IgG-2scFv, scFv4-Jg, Zybody, DVI-IgG, Diabody-CH3, a triple body, a miniantibody, a minibody, a TriBi minibody, scFv-CH3 KIH, Fab-scFv, a F(ab′)2-scFv2, a scFv-KIH, a Fab-scFv-Fc, a tetravalent HCAb, a scDiabody-Fc, a Diabody-Fc, a tandem scFv-Fc, a VHH-Fc, a tandem VHH-Fc, a VHH-Fc KiH, a Fab-VHH-Fc, an Intrabody, a dock and lock, an ImmTAC, an IgG-IgG conjugate, a Cov-X-Body, a scFv1-PEG-scFv2, an Adnectin, a DARPin, a fibronectin, and a DEP conjugate. See, e.g., Spiess et al., Mol. Immunol. 67:95-106, 2015, incorporated in its entirety herewith, for a description of these elements. Non-limiting examples of an antigen-binding fragment of an antibody include an Fv fragment, a Fab fragment, a F(ab′)₂ fragment, and a Fab′ fragment. Additional examples of an antigen-binding fragment of an antibody is an antigen-binding fragment of an IgG (e.g., an antigen-binding fragment of IgG1, IgG2, IgG3, or IgG4) (e.g., an antigen-binding fragment of a human or humanized IgG, e.g., human or humanized IgG1, IgG2, IgG3, or IgG4); an antigen-binding fragment of an IgA (e.g., an antigen-binding fragment of IgAQ1 or IgA2) (e.g., an antigen-binding fragment of a human or humanized IgA, e.g., a human or humanized IgAQ1 or IgA2); an antigen-binding fragment of an IgD (e.g., an antigen-binding fragment of a human or humanized IgD); an antigen-binding fragment of an IgE (e.g., an antigen-binding fragment of a human or humanized IgE); or an antigen-binding fragment of an IgM (e.g., an antigen-binding fragment of a human or humanized IgM).

A “Fv” fragment includes a non-covalently-linked dimer of one heavy chain variable domain and one light chain variable domain.

A “Fab” fragment includes, the constant domain of the light chain and the first constant domain (C_{H1}) of the heavy chain, in addition to the heavy and light chain variable domains of the Fv fragment.

A “F(ab′)₂” fragment includes two Fab fragments joined, near the hinge region, by disulfide bonds.

A “dual variable domain immunoglobulin” or “DVD-Ig” refers to multivalent and multispecific binding proteins as described, e.g., in DiGiammarino et al., Methods Mol. Biol. 899:145-156, 2012; Jakob et al., MABs 5:358-363, 2013; and U.S. Pat. Nos. 7,612,181; 8,258,268; 8,586,714; 8,716,450; 8,722,855; 8,735,546; and 8,822,645, each of which is incorporated by reference in its entirety.

DARTs are described in, e.g., Garber, Nature Reviews Drug Discovery 13:799-801, 2014.

Additional aspects of antigen-binding domains are known in the art.

In some embodiments of any of the proteins described herein, the K_{D} of the antigen-binding domain at a physiological pH (e.g., pH 7.4) is between about 1 pM to about 5 μM (e.g., about 1 pM to about 2 μM, about 1 pM to about 1 pM, about 1 pM to about 500 nM, about 1 pM to about 250 nM, about 1 pM to about 200 nM, about 1 pM to about 100 nM, about 1 pM to about 50 nM, about 1 pM to about 10 nM, about 1 pM to about 1 nM, about 1 pM to about 800 pM, about 1 pM to about 600 pM, about 1 pM to about 400 pM, about 1 pM to about 200 pM, about 1 pM to about 100 pM, about 1 pM to about 50 pM, about 50 pM to about 5 μM, about 50 pM to about 2 μM, about 50 pM to about 1 μM, about 50 pM to about 500 nM, about 50 pM to about 250 nM, about 50 pM to about 200 nM, about 50 pM to about 100 nM, about 50 pM to about 50 nM, about 50 pM to about 10 nM, about 50 pM to about 1 nM, about 50 pM to about 800 pM, about 50 pM to about 600 pM, about 50 pM to about 400 pM, about 50 pM to about 200 pM, about 50 pM to about 100 pM, about 100 pM to about 5 μM, about 100 pM to about 2 μM, about 100 pM to about 1 μM, about 100 pM to about 500 nM, about 100 pM to about 250 nM, about 100 pM to about 200 nM, about 100 pM to about 100 nM, about 100 pM to about 50 nM, about 100 pM to about 10 nM, about 100 pM to about 1 nM, about 100 pM to about 800 pM, about 100 pM to about 600 pM, about 100 pM to about 400 pM, about 100 pM to about 200 pM, about 200 pM to about 5 μM, about 200 pM to about 2 μM, about 200 pM to about 1 μM, about 200 pM to about 500 nM, about 200 pM to about 250 nM, about 200 pM to about 200 nM, about 200 pM to about 100 nM, about 200 pM to about 50 nM, about 200 pM to about 10 nM, about 200 pM to about 1 nM, about 200 pM to about 800 pM, about 200 pM to about 600 pM, about 200 pM to about 400 pM, about 400 pM to about 5 μM, about 400 pM to about 2 μM, about 400 pM to about 1 pM, about 400 pM to about 500 nM, about 400 pM to about 250 nM, about 400 pM to about 200 nM, about 400 pM to about 100 nM, about 400 pM to about 50 nM, about 400 pM to about 10 nM, about 400 pM to about 1 nM, about 400 pM to about 800 pM, about 400 pM to about 600 pM, about 600 pM to about 5 μM, about 600 pM to about 2 μM, about 600 pM to about 1 pM, about 600 pM to about 500 nM, about 600 pM to about 250 nM, about 600 pM to about 200 nM, about 600 pM to about 100 nM, about 600 pM to about 50 nM, about 600 pM to about 10 nM, about 600 pM to about 1 nM, about 600 pM to about 800 pM, about 800 pM to about 5 μM, about 800 pM to about 2 μM, about 800 pM to about 1 μM, about 800 pM to about 500 nM, about 800 pM to about 250 nM, about 800 pM to about 200 nM, about 800 pM to about 100 nM, about 800 pM to about 50 nM, about 800 pM to about 10 nM, about 800 pM to about 1 nM, about 1 nM to about 5 μM, about 1 nM to about 2 μM, about 1 nM to about 1 pM, about 1 nM to about 500 nM, about 1 nM to about 250 nM, about 1 nM to about 200 nM, about 1 nM to about 100 nM, about 1 nM to about 50 nM, about 1 nM to about 10 nM, about 10 nM to about 5 μM, about 10 nM to about 2 μM, about 10 nM to about 1 μM, about 10 nM to about 500 nM, about 10 nM to about 250 nM, about 10 nM to about 200 nM, about 10 nM to about 100 nM, about 10 nM to about 50 nM, about 50 nM to about 5 μM, about 50 nM to about 2 μM, about 50 nM to about 1 μM, about 50 nM to about 500 nM, about 50 nM to about 250 nM, about 50 nM to about 200 nM, about 50 nM to about 100 nM, about 100 nM to about 5 μM, about 100 nM to about 2 μM, about 100 nM to about 1 μM, about 100 nM to about 500 nM, about 100 nM to about 250 nM, about 100 nM to about 200 nM, about 200 nM to about 5 μM, about 200 nM to about 2 μM, about 200 nM to about 1 μM, about 200 nM to about 500 nM, about 200 nM to about 250 nM, about 250 nM to about 5 μM, about 250 nM to about 2 μM, about 250 nM to about 1 pM, about 250 nM to about 500 nM, about 500 nM to about 5 μM, about 500 nM to about 2 μM, about 500 nM to about 1 μM, about 1 μM to about 5 μM, about 1 μM to about 2 μM, or about 2 μM to about 5 μM.

In some embodiments, the protein comprising an antigen-binding domain can further comprise a conjugated cytotoxic or cytostatic agent.

Examples of cytotoxic or cytostatic agent include, but are not limited to auristatins (e.g., auristatin E, auristatin F, MMAE and MMAF), auromycins, maytansinoids, ricin, duocarmycins, dolastatins, doxorubicin, daunorubicin, taxols (e.g., paclitaxel), cisplatin, camptothecin, CC-1065, amatoxins, ethidium bromide, mitomycin, etoposide, tenoposide, vincristine, vinblastine, colchicine, actinomycin, calicheamicin, Pseudomonas exotoxin (PE), diphtheria toxin (DT)

### Expression of a Protein Comprising an Antigen-Binding Domain in a Cell

Also provided herein are methods of generating a recombinant cell that expresses an protein (e.g., any of the proteins including an antigen-binding domain described herein) that include: introducing into a cell a nucleic acid encoding the protein to produce a recombinant cell; and culturing the recombinant cell under conditions sufficient for the expression of the protein. In some embodiments, the introducing step includes introducing into a cell an expression vector including a nucleic acid encoding the protein to produce a recombinant cell.

Any of the proteins described herein can be produced by any cell, e.g., a eukaryotic cell or a prokaryotic cell. As used herein, the term “eukaryotic cell” refers to a cell having a distinct, membrane-bound nucleus. Such cells may include, for example, mammalian (e.g., rodent, non-human primate, or human), insect, fungal, or plant cells. In some embodiments, the eukaryotic cell is a yeast cell, such as Saccharomyces cerevisiae. In some embodiments, the eukaryotic cell is a higher eukaryote, such as mammalian, avian, plant, or insect cells. As used herein, the term “prokaryotic cell” refers to a cell that does not have a distinct, membrane-bound nucleus. In some embodiments, the prokaryotic cell is a bacterial cell.

Methods of culturing cells are well known in the art. Cells can be maintained in vitro under conditions that favor proliferation, differentiation, and growth. Briefly, cells can be cultured by contacting a cell (e.g., any cell) with a cell culture medium that includes the necessary growth factors and supplements to support cell viability and growth.

Methods of introducing nucleic acids and expression vectors into a cell (e.g., a eukaryotic cell) are known in the art. Non-limiting examples of methods that can be used to introduce a nucleic acid into a cell include lipofection, transfection, electroporation, microinjection, calcium phosphate transfection, dendrimer-based transfection, cationic polymer transfection, cell squeezing, sonoporation, optical transfection, impalection, hydrodynamic delivery, magnetofection, viral transduction (e.g., adenoviral and lentiviral transduction), and nanoparticle transfection.

Provided herein are methods that further include isolation of the protein from a cell (e.g., a eukaryotic cell) using techniques well-known in the art (e.g., ammonium sulfate precipitation, polyethylene glycol precipitation, ion-exchange chromatography (anion or cation), chromatography based on hydrophobic interaction, metal-affinity chromatography, ligand-affinity chromatography, and size exclusion chromatography).

### Compositions

Also provided herein are compositions (e.g., pharmaceutical compositions) that include at least one of any of the proteins described herein and at least one pharmaceutically acceptable excipient. In some embodiments, the compositions (e.g., pharmaceutical compositions) can be disposed in a sterile vial or a pre-loaded syringe.

In some embodiments, the compositions (e.g., pharmaceutical compositions) are formulated for different routes of administration (e.g., intravenous, subcutaneous, intramuscular, or intratumoral). In some embodiments, the compositions (e.g., pharmaceutical compositions) can include a pharmaceutically acceptable carrier (e.g., phosphate buffered saline). Single or multiple administrations of any of the pharmaceutical compositions described herein can be given to a subject depending on, for example: the dosage and frequency as required and tolerated by the subject. A dosage of the pharmaceutical composition should provide a sufficient quantity of the protein to effectively treat or ameliorate conditions, diseases, or symptoms.

Also provided herein are methods of treating a subject having a cancer (e.g., any of the cancers described herein) that include administering a therapeutically effective amount of at least one of any of the compositions or pharmaceutical compositions provided herein.

### Kits

Also provided herein are kits that include any of the proteins described herein, any of the compositions described herein, or any of the pharmaceutical compositions described herein. In some embodiments, the kits can include instructions for performing any of the methods described herein. In some embodiments, the kits can include at least one dose of any of the compositions (e.g., pharmaceutical compositions) described herein. In some embodiments, the kits can provide a syringe for administering any of the pharmaceutical compositions described herein. In some embodiments, the kits can include instructions for administration of the pharmaceutical composition to a human subject.

### Methods of Treatment

Provided herein are methods of treating a subject in need thereof that include: administering a therapeutically effective amount of any of the pharmaceutical compositions described herein or any of the proteins comprising an antigen-binding domain described herein to a subject identified as being in need thereof.

In some embodiments, the subject is further administered one or more additional therapeutic agents. In some embodiments, the one or more additional therapeutic agents is administered to the subject at approximately the same time as any of the proteins described herein are administered to the subject. In some embodiments, the one or more additional therapeutic agents are administered to the subject after the administration of any of the proteins described herein to the subject. In some embodiments, the one or more additional therapeutic agents are administered to the subject before the administration of any of the proteins described herein to the subject.

Also provided herein are methods of inducing or increasing (e.g., at least a 1% increase, at least a 5% increase, at least a 10% increase, at least a 15% increase, at least a 20% increase, at least a 30% increase, at least a 40% increase, or at least a 50% increase) internalization of a target protein (specifically bound by the antigen-binding domain of the protein) that includes contacting the mammalian cell with the protein, e.g., as compared to the level of internalization of the target protein in the absence of the protein. In some embodiments, the mamnmalian cell is in vivo. In some embodiments, the mammalian cell is in vitro.

Also provided are methods of inhibiting (e.g., at least a 1% decrease, at least a 5% decrease, at least a 10% decrease, at least a 20% decrease, at least a 30% decrease, at least a 40% decrease, at least a 50% decrease, at least a 60% decrease, at least a 70% decrease, at least a 80% decrease, or at least a 90% decrease) an activity of a target protein (specifically bound by the antigen-binding domain of the protein) in a mammalian cell that includes contacting the target protein with the protein, e.g., as compared to the level of activity of the target protein in the absence of the protein. In some embodiments, the mammalian cell is in vivo. In some embodiments, the mammalian cell is in vitro.

Also provided are methods of reducing (e.g., at least a 1% decrease, at least a 5% decrease, at least a 10% decrease, at least a 20% decrease, at least a 30% decrease, at least a 40% decrease, at least a 50% decrease, at least a 60% decrease, at least a 70% decrease, at least a 80% decrease, or at least a 90% decrease) the amount of the target protein (specifically bound by the antigen-binding domain of the protein) in a mammalian cell that includes contacting the target protein with the protein, e.g., as compared to the amount of the target protein in the absence of the protein. In some embodiments, the mammalian cell is in vivo. In some embodiments, the mammalian cell is in vitro.

Also provided are methods of inducing (e.g., at least a 1% increase, at least a 5% increase, at least a 10% increase, at least a 15% increase, at least a 20% increase, at least a 30% increase, at least a 40% increase, or at least a 50% increase) cell death in a mammalian cell comprising the target protein (specifically bound by the antigen-binding domain of the protein) that includes contacting the cell with the protein, e.g., as compared to the level of cell death in a similar mammalian cell not contacted with the protein. In some embodiments, the mammalian cell is in vivo. In some embodiments, the mammalian cell is in vitro.

### Methods of Screening

Also provided herein are methods for screening for a protein that forms a covalent bond with a target protein in a mammalian cell that include contacting the target protein with any of the proteins that include an antigen-binding domain described herein and determining whether a covalent bond has been formed between the protein and the target protein. In some embodiments, the method further includes determining whether the mammalian cell has internalized the protein. In some embodiments, the method further includes determining whether the contacting has inhibiting an activity of the target protein and/or determining whether the contacting has induced cell death of the mammalian cell. In some embodiments, the method further includes testing the protein in an animal model of a disease.

### Protein-Protein Conjugates

Some embodiments provide a protein-protein conjugate comprising a first protein A and a second protein B, wherein the protein-protein conjugate has the structure:

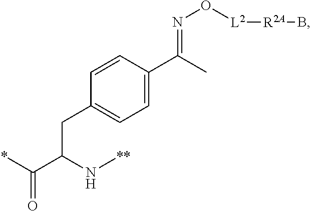

wherein the first protein A comprises an antigen-binding domain, wherein the antigen-binding domain comprises a modified phenylalanine residue, wherein:
* and ** represent the points of connection of the modified phenylalanine residue to the antigen-binding domain of the first protein A;
L² is a C2-C16 alkylene optionally substituted with 1-2 substituents independently selected from halogen and C3-C6 cycloalkyl, and wherein 1-6 alkylene units are optionally replaced by O, N, S, C3-C10 cycloalkyl, phenyl, 5-12 membered heteroaryl, and 4-14 membered heterocyclyl;
R^{2A} is

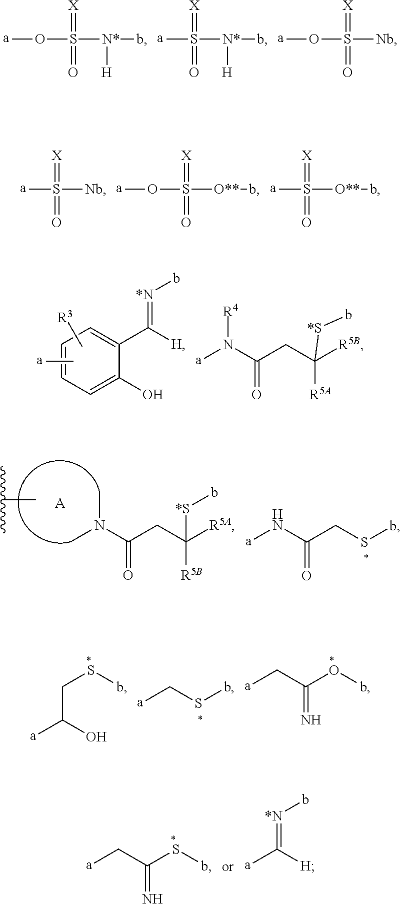

“a” represents the connection of R^{2A} to L², “b” represents the connection of R^{2A} to protein B, N* is a nitrogen atom of a lysine residue of protein B, S* is a sulfur atom of a cysteine residue of protein B, O* is an oxygen atom from a serine residue or a threonine residue of protein B, Nb is the nitrogen atom of a histidine residue of protein B and the connection of R^{2A} to protein B, and O** is an oxygen atom from a tyrosine residue of protein B;
X is O or NR^{X};
R^{X} is hydrogen, C1-C6 alkyl, or C3-C6 cycloalkyl;
R³ is halogen or C1-C6 alkyl;
R⁴ is hydrogen or C1-C6 alkyl;
R^{4A} is C1-C6 alkyl or C3-C6 cycloalkyl;
R^{5A} and R^{5B} are independently hydrogen, halogen, or C1-C6 alkyl; or
R^{5A} and R^{5B} together with the carbon atom to which they are attached form a cyclopropyl;
Ring A is a 4-10 membered heterocyclyl;
wherein the antigen-binding domain of the first protein A specifically binds to the second protein B.

In some embodiments, L² is a C2-C16 alkylene optionally substituted with 1-2 substituents independently selected from halogen and C3-C6 cycloalkyl, and wherein 1-6 alkylene units are optionally replaced by O, N, S, C3-C10 cycloalkyl, phenyl, 5-12 membered heteroaryl, —C(O)—, and 4-14 membered heterocyclyl.

In some embodiments, L² is a C1-C16 alkylene optionally substituted with 1-2 substituents independently selected from halogen and C3-C6 cycloalkyl, and wherein 1-6 alkylene units are optionally replaced by O, N, S, C3-C10 cycloalkyl, phenyl, 5-12 membered heteroaryl, —C(O)—, and 4-14 membered heterocyclyl.

In some embodiments, the modified phenylalanine residue is present in a CDR of the antigen-binding domain. In some embodiments, the CDR is a heavy chain CDR. In some embodiments, the CDR is a light chain CDR.

In some embodiments, the modified phenylalanine residue is present in a framework region of the antigen-binding domain.

Some embodiments provide a protein-protein conjugate comprising a first protein A and a second protein B, wherein the protein-protein conjugate has the structure:

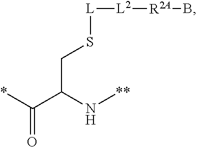

wherein the first protein A comprises an antigen-binding domain, wherein the antigen-binding domain comprises a modified cysteine residue, wherein:
* and ** represent the points of connection of the modified cysteine residue to the antigen-binding domain;
L is a bond,

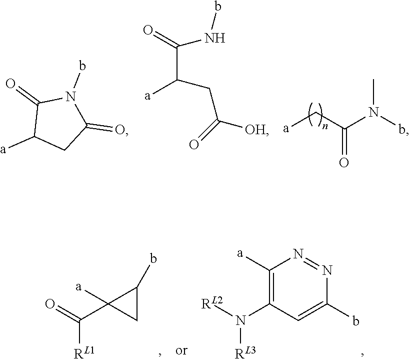

wherein a represents the point of connection of L to the sulfur atom of the modified cysteine residue and b represents the point of connection of L to L²;
n is 1 or 2;
R^{L1}, R^{L2}, and R^{L3}, are each independently selected C1-C10 alkyl;
L² is a C2-C16 alkylene optionally substituted with 1-2 substituents independently selected from halogen and C3-C6 cycloalkyl, and wherein 1-6 alkylene units are optionally replaced by O, N, S, C3-C10 cycloalkyl, phenyl, 5-12 membered heteroaryl, and 4-14 membered heterocyclyl;
R^{2A} is

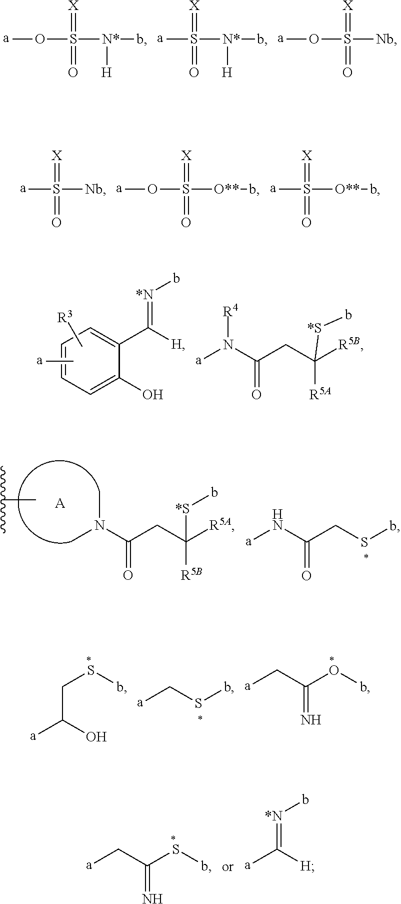

“a” represents the connection of R^{2A} to L², “b” represents the connection of R^{2A} to protein B, N* is a nitrogen atom of a lysine residue of protein B, S* is a sulfur atom of a cysteine residue of protein B, O* is an oxygen atom from a serine residue or a threonine residue of protein B, Nb is the nitrogen atom of a histidine residue of protein B and the connection of R^{2A} to protein B, and O** is an oxygen atom from a tyrosine residue of protein B;
X is O or NR^{X};
R^{X} is hydrogen, C1-C6 alkyl, or C3-C6 cycloalkyl;
R³ is halogen or C1-C6 alkyl;
R⁴ is hydrogen or C1-C6 alkyl;
R^{4A} is C1-C6 alkyl or C3-C6 cycloalkyl;
R^{5A} and R^{5B} are independently hydrogen, halogen, or C1-C6 alkyl; or
R^{5A} and R^{5B} together with the carbon atom to which they are attached form a cyclopropyl;
Ring A is a 4-10 membered heterocyclyl,
wherein the antigen-binding domain of the first protein A specifically binds to the second protein B.

In some embodiments, L² is a C2-C16 alkylene optionally substituted with 1-2 substituents independently selected from halogen and C3-C6 cycloalkyl, and wherein 1-6 alkylene units are optionally replaced by O, N, S, C3-C10 cycloalkyl, phenyl, 5-12 membered heteroaryl, —C(O)—, and 4-14 membered heterocyclyl.

In some embodiments, L² is a C1-C16 alkylene optionally substituted with 1-2 substituents independently selected from halogen and C3-C6 cycloalkyl, and wherein 1-6 alkylene units are optionally replaced by O, N, S, C3-C10 cycloalkyl, phenyl, 5-12 membered heteroaryl, —C(O)—, and 4-14 membered heterocyclyl.

In some embodiments, it is provided that the structure does not contain a GLP-1 peptide or variant thereof, optionally wherein the structure does not contain a modified GLP-1 peptide, optionally wherein the structure does not contain a peptide as described in WO 2025/076010.

In some embodiments, the modified cysteine residue is present in a CDR of the antigen-binding domain. In some embodiments, the CDR is a heavy chain CDR. In some embodiments, the CDR is a light chain CDR.

In some embodiments, the modified cysteine residue is present in a framework region of the antigen-binding domain.

In some embodiments, the first protein A is an antibody. In some embodiments, the antibody is a human antibody, a humanized antibody, or a veneered antibody. In some embodiments, the antibody is a human IgG1, human IgG2, human IgG3, or human IgG4 antibody.

In some embodiments, the first protein A is or comprises a single chain Fv (scFv), a VHH, a VNAR, a DARpin, a single domain antibody (sdAb), an Adnectin/Centyrin, an Affibody, a Knottin, a bicyclic peptide, or a cyclic peptide.

In some embodiments, the first protein A further comprises a conjugated cytotoxic or cytostatic agent.

In some embodiments, the first protein A comprises a radioisotope.

In some embodiments, the second protein B comprises an extracellular domain, and the antigen-binding domain specifically binds to the extracellular domain.

### Methods of Making a Protein

Some embodiments provide a method of making a protein, wherein the antigen-binding domain comprises a modified phenylalanine residue, the modified phenylalanine residue having the structure:

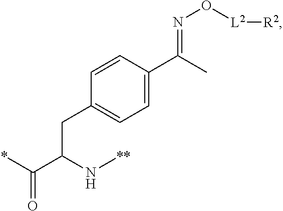

the method comprising contacting
a compound having the structure Z—R² with
a protein comprising an antigen-binding domain, wherein the antigen-binding domain comprising an oxime, the oxime having the structure:

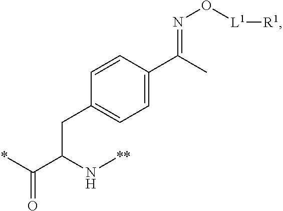

wherein:
Z reacts with -L¹- to form -L²-, wherein when R¹ is azido or tetrazinyl, then Z is a C2-C3 alkyne or an optionally substituted C8-C12 cycloalkyne, and when R¹ is a C2-C3 alkyne or an optionally substituted C8-C12 cycloalkyne, then Z is azido or tetrazinyl;
* and ** represent the points of connection of the oxime to the antigen-binding domain;
L² is a C2-C16 alkylene optionally substituted with 1-2 substituents independently selected from halogen and C3-C6 cycloalkyl, and wherein 1-6 alkylene units are optionally replaced by O, N, S, C3-C10 cycloalkyl, phenyl, 5-12 membered heteroaryl, and 4-14 membered heterocyclyl;
R² is

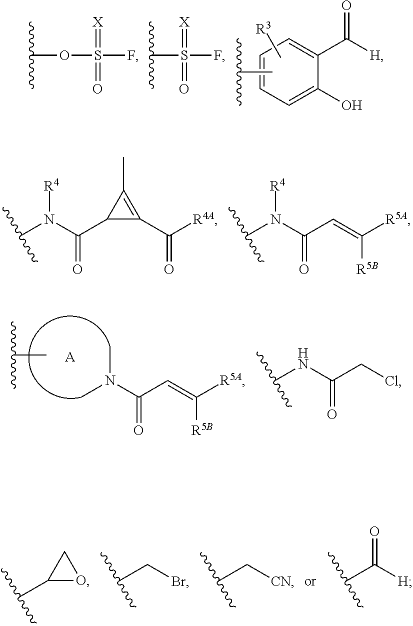

X is O or NR^{X};
R^{X} is hydrogen, C1-C6 alkyl, or C3-C6 cycloalkyl;
R³ is halogen or C1-C6 alkyl;
R⁴ is hydrogen or C1-C6 alkyl;
R^{4A} is C1-C6 alkyl or C3-C6 cycloalkyl;
R^{5A} and R^{5B} are independently hydrogen, halogen, or C1-C6 alkyl; or
R^{5A} and R^{5B} together with the carbon atom to which they are attached form a cyclopropyl;
Ring A is a 4-10 membered heterocyclyl;
L¹ is a bond or a C1-C6 alkylene optionally substituted with 1-2 substituents independently selected from halogen and C3-C6 cycloalkyl, and wherein 1-2 alkylene units are optionally replaced by O, N, C3-C6 cycloalkyl, or phenyl; and
R¹ is azido, tetrazinyl, a C2-C3 alkyne, or an optionally substituted C8-C12 cycloalkyne.

In some embodiments, L² is a C2-C16 alkylene optionally substituted with 1-2 substituents independently selected from halogen and C3-C6 cycloalkyl, and wherein 1-6 alkylene units are optionally replaced by O, N, S, C3-C10 cycloalkyl, phenyl, 5-12 membered heteroaryl, —C(O)—, and 4-14 membered heterocyclyl.

In some embodiments, L² is a C1-C16 alkylene optionally substituted with 1-2 substituents independently selected from halogen and C3-C6 cycloalkyl, and wherein 1-6 alkylene units are optionally replaced by O, N, S, C3-C10 cycloalkyl, phenyl, 5-12 membered heteroaryl, —C(O)—, and 4-14 membered heterocyclyl.

In some embodiments, the modified phenylalanine residue is present in a CDR of the antigen-binding domain. In some embodiments, the CDR is a heavy chain CDR. In some embodiments, the CDR is a light chain CDR.

In some embodiments, the modified phenylalanine residue is present in a framework region of the antigen-binding domain.

In some embodiments, the protein is an antibody. In some embodiments, the antibody is a human antibody, a humanized antibody, or a veneered antibody. In some embodiments, the antibody is a human IgG1, human IgG2, human IgG3, or human IgG4 antibody.

In some embodiments, the protein is or comprises a single chain Fv (scFv), a VHH, a VNAR, a DARpin, a single domain antibody (sdAb), an Adnectin/Centyrin, an Affibody, a Knottin, a bicyclic peptide, or a cyclic peptide.

In some embodiments, the protein further comprises a conjugated cytotoxic or cytostatic agent.

In some embodiments, the protein comprises a radioisotope. Examples of radioisotopes include, but are not limited to At²¹¹, I¹³¹, I¹²⁵, Y⁹⁰, Re¹⁸⁶, Re¹⁸⁸, Sm¹⁵³, Bi²¹² or ²¹³, P³² and radioactive isotopes of Lu including Lu¹⁷⁷.

In some embodiments, the antigen-binding domain specifically binds to a target protein.

In some embodiments, the target protein comprises an extracellular domain, and the antigen-binding domain specifically binds to the extracellular domain.

### EXAMPLES

### Compound Preparation

The compounds disclosed herein can be prepared in a variety of ways using commercially available starting materials, compounds known in the literature, or from readily prepared intermediates, by employing standard synthetic methods and procedures either known to those skilled in the art, or in light of the teachings herein. The synthesis of the compounds disclosed herein can be achieved by generally following the schemes provided herein, with modification for specific desired substituents.

Standard synthetic methods and procedures for the preparation of organic molecules and functional group transformations and manipulations can be obtained from the relevant scientific literature or from standard textbooks in the field. Although not limited to any one or several sources, classic texts such as R. Larock, Comprehensive Organic Transformations, VCH Publishers (1989); L. Fieser and M. Fieser, Fieser and Fieser's Reagents for Organic Synthesis, John Wiley and Sons (1994); Smith, M. B., March, J., March's Advanced Organic Chemistry: Reactions, Mechanisms, and Structure, 5th edition, John Wiley & Sons: New York, 2001; and Greene, T. W., Wuts, P. G. M., Protective Groups in Organic Synthesis, 3rd edition, John Wiley & Sons: New York, 1999, are useful and recognized reference textbooks of organic synthesis known to those in the art. The following descriptions of synthetic methods are designed to illustrate, but not to limit, general procedures for the preparation of compounds of the present disclosure.

The synthetic processes disclosed herein can tolerate a wide variety of functional groups; therefore, various substituted starting materials can be used. The starting materials that can be used for the synthesis can be synthesized according to known literature procedures or obtained from commercial sources, such as, but not limited to, Sigma-Aldrich, Fluka, Acros Organics, Alfa Aesar, VWR Scientific, and the like.

The following general reaction schemes are provided for illustrative purposes only to exemplify chemical transformations useful for preparing various embodiments of the chemical entities described herein (e.g., linkers) as well as intermediates used in preparing the same.

### 1. General Synthesis of Alkoxylamine Fluorosulfates

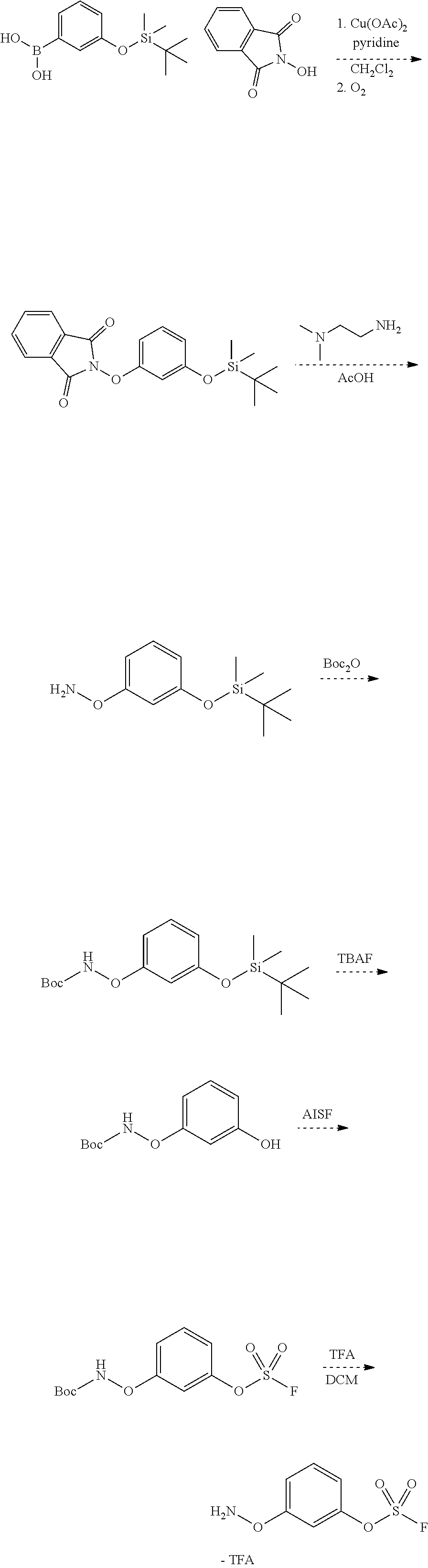

2. Conjugation of VHH from Engineered pAF to Fluorosulfate

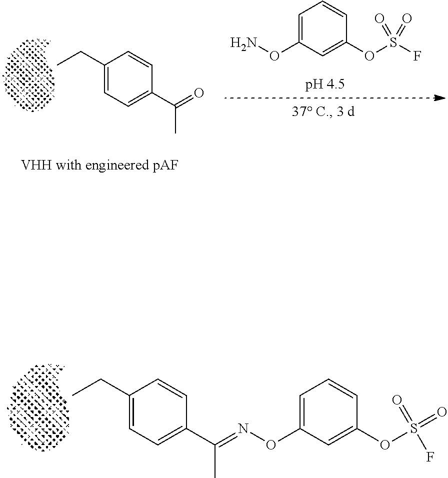 [SMILES: CCc1ccc(C(C)=O)cc1 ; NOc1cccc(OS(=O)(=O)F)c1 ; CCc1ccc(/C(C)=N/Oc2cccc(OS(=O)(=O)F)c2)cc1]

3. Conjugation of VHH with Alkynyl Linker from pAF, Followed by Cu Mediated AAC to Introduce Fluorosulfate

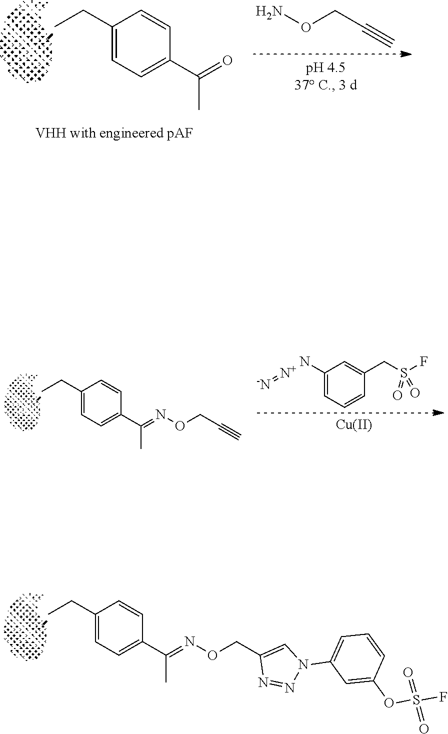

### 4. Preparation of Maleimide Fluorosulfate

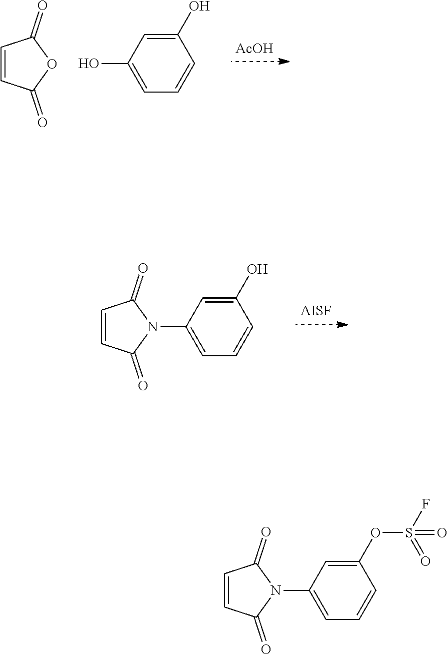

5. Conjugation of Maleimide Fluorosulfate with Unpaired Cys

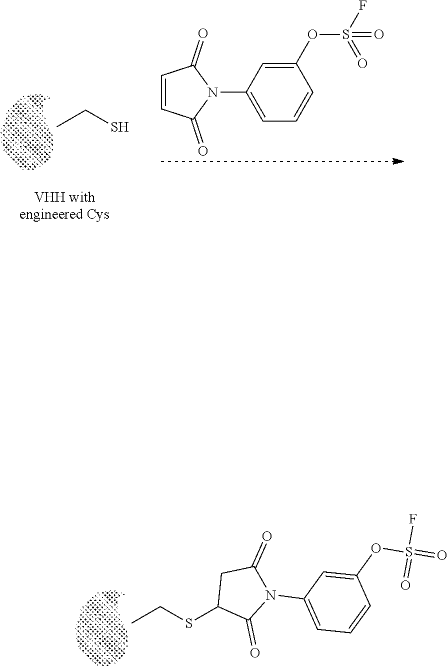

### 6. Synthesis of Alkynyl Cyclopropenone

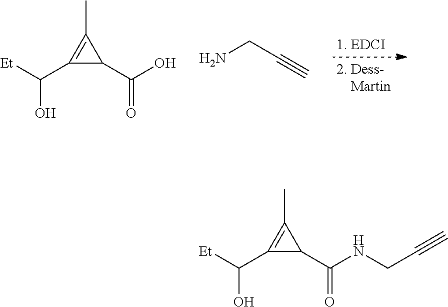

7. Conjugation of Unpaired Cys with Alkynyl Cyclopropenone and Cu AAc Click Reaction

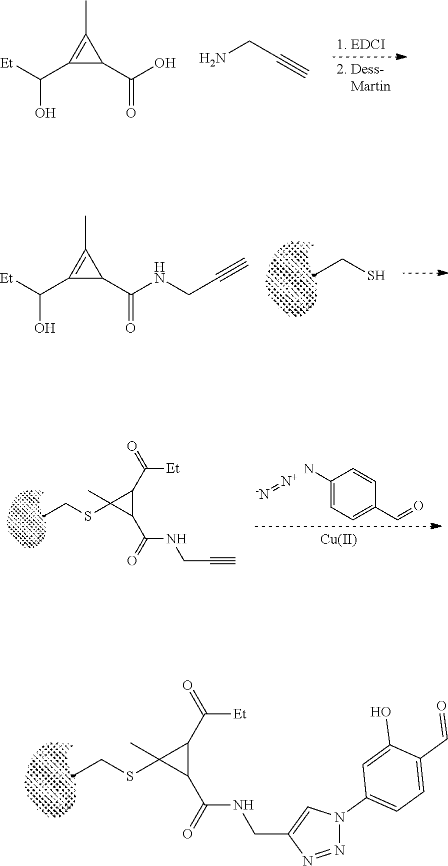

8. Conjugation of Unpaired Cys with

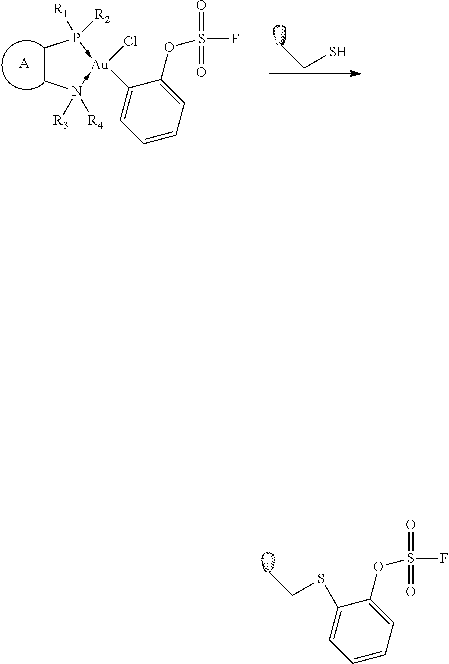

### Synthesis of Linkers

### Example 1. Synthesis of Au-1-3-Me

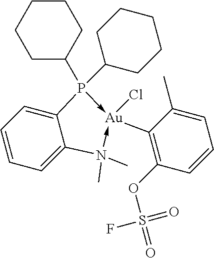

Step 1: To a stirred solution of 2-iodo-3-methylphenol (250 mg, 1.068 mmol, 1 equiv) and 3-(fluorosulfonyl)-1,2-dimethylimidazol-1-ium triflate (525.96 mg, 1.602 mmol, 1.5 equiv) in MeCN (3 mL) was added TEA (216.19 mg, 2.136 mmol, 2.00 equiv) dropwise at 0° C. under nitrogen atmosphere. The resulting mixture was concentrated under reduced pressure. The residue was purified by reversed-phase flash chromatography with the following conditions: column, C18 silica gel; mobile phase, MeCN in Water (0.1% FA), 10% to 95% gradient in 10 min; detector, UV 254 nm. This resulted in 2-iodo-3-methylphenyl sulfurofluoridate (120 mg, 35.54% yield) as a light yellow oil.

Step 2: To a stirred solution of Silver Hexafluoroantimonate(V) (130.46 mg, 0.380 mmol, 1 equiv) in DCM (3 mL) at 0° C. under nitrogen atmosphere. To the above mixture was added 2-iodo-3-methylphenyl sulfurofluoridate (120 mg, 0.380 mmol, 1 equiv) and 2-[(chloroaurio)dicyclohexyl-lambda5-phosphanyl]-N,N-dimethylaniline (208.76 mg, 0.380 mmol, 1 equiv) in portions at 0° C. The resulting mixture was stirred at room temperature for additional 3 h. The resulting mixture was filtered, the filter cake was washed with DCM (1 mL×3). The filtrate was concentrated under reduced pressure, and added DCM (0.6 mL) to the concentrated filtrate. The system was purified by trituration with MTBE (1.8 mL). This resulted in 2-chloro-3,3-dicyclohexyl-2-{2-[(fluorosulfonyl)oxy]-6-methylphenyl}-1,1-dimethyl-2H,3H-3lambda5-benzo[d]1-aza-3lambda5-phospha-2-auracyclopentan-1-ium-3-ylium-2,2-diuide (70 mg, 24.95% yield, 97.9% purity) as a light brown solid. LCMS:(ES, m/z): [M]⁺=738.0.

¹H NMR— (300 MHz, DMSO-d₆, ppm) δ 8.41 (dd, J=8.6, 4.4 Hz, 1H), 8.13-7.97 (m, 2H), 7.82 (td, J=7.6, 2.5 Hz, 1H), 7.57-7.27 (m, 3H), 3.89-3.36 (m, 6H), 3.27 (s, 1H), 2.83 (d, J=12.6 Hz, 1H), 2.64 (s, 3H), 1.98 (d, J=14.8 Hz, 2H), 1.841-1.58 (m, 6H), 1.55-1.14 (m, 8H), 1.00 (t, J=12.1 Hz, 2H), 0.74 (s, 1H), 0.26 (s, 1H).

### Example 2. Synthesis of Au-1-4-F

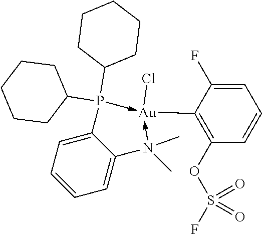

Step 1: To a stirred solution/mixture of 3-fluoro-2-iodophenol (100 mg, 0.420 mmol, 1 equiv) and 3-(fluorosulfonyl)-1,2-dimethylimidazol-1-ium triflate (165.50 mg, 0.504 mmol, 1.2 equiv) in DCM (4 mL) was added TEA (85.04 mg, 0.840 mmol, 2 equiv) dropwise at 0° C. The resulting mixture was stirred at room temperature for additional 1 h. The reaction was monitored by TLC. The residue was purified by silica gel column chromatography, eluted with PE/EA (5:1) to afford 3-fluoro-2-iodophenyl sulfurofluoridate (40 mg, 29.75% yield) as a colorless oil.

Step 1: To a stirred mixture of AgSbF₆ (42.95 mg, 0.125 mmol, 1 equiv) in DCM (3 mL) were added 3-fluoro-2-iodophenyl sulfurofluoridate (40 mg, 0.125 mmol, 1 equiv) and 3-fluoro-2-iodophenyl sulfurofluoridate (40 mg, 0.125 mmol, 1 equiv) in portions at 0° C. The resulting mixture was stirred at 25° C. for additional 2 h. The resulting mixture was filtered, and the filter cake was washed with DCM. The filtrate was concentrated under reduced pressure, and added DCM (0.6 mL) to the concentrated filtrate. The system was purified by trituration with MTBE (1.8 mL). This resulted in 2-chloro-3,3-dicyclohexyl-2-{2-fluoro-6-[(fluorosulfonyl) oxy]phenyl}-1,1-dimethyl-2H,3H-3lambda5-benzo[d]1-aza-3lambda5-phospha-2-auracyclopentan-1-ium-3-ylium-2,2-diuide (40 mg, 43.07% yield, 90.2% purity) as an off-white solid. LCMS: (ES, m/z): [M]⁺=742.0.

¹H NMR (300 MHz, DMSO-d₆, ppm) δ 8.44 (dd, J=8.6, 4.4 Hz, 1H), 8.18-8.06 (m, 2H), 7.86 (dt, J=8.0, 4.0 Hz, 1H), 7.80-7.60 (m, 2H), 7.60-7.49 (m, 1H), 3.60 (d, J=15.1 Hz, 6H), 3.22 (d, J=11.2 Hz, 1H), 3.03 (s, 1H), 1.86 (s, 3H), 1.67 (s, 7H), 1.35 (dd, J=29.5, 17.1 Hz, 6H), 1.02 (d, J=11.5 Hz, 2H), 0.67 (s, 1H), 0.43 (s, 1H).

### Example 3. Synthesis of Au-2-2-CN

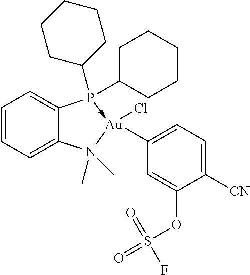

Step 1: To a stirred solution of 2-hydroxy-4-iodobenzonitrile (250 mg, 1.020 mmol, 1 equiv) and 3-(fluorosulfonyl)-1,2-dimethylimidazol-1-ium triflate (502.38 mg, 1.530 mmol, 1.5 equiv) in MeCN (3 mL) was added TEA (206.50 mg, 2.040 mmol, 2 equiv) dropwise at 0° C. under nitrogen atmosphere. The resulting mixture was stirred at room temperature for additional 30 min. The resulting mixture was concentrated under reduced pressure. The residue was purified by reversed-phase flash chromatography with the following conditions: column, C18 silica gel; mobile phase, MeCN in Water (0.1% FA), 10% to 90% gradient in 10 min; detector, UV 254 nm. This resulted in 2-cyano-5-iodophenyl sulfurofluoridate (130 mg, 38.95% yield) as a light yellow solid.

Step 2: To a stirred solution of Silver Hexafluoroantimonate(V) (136.58 mg, 0.397 mmol, 1 equiv) in DCM (3 mL) at 0° C. under nitrogen atmosphere. To the above mixture was added 2-cyano-5-iodophenyl sulfurofluoridate (130 mg, 0.397 mmol, 1 equiv) and 2-[(chloroaurio)dicyclohexyl-lambda5-phosphanyl]-N,N-dimethylaniline (218.56 mg, 0.397 mmol, 1 equiv) in portions at 0° C. The resulting mixture was stirred at room temperature for additional overnight. The resulting mixture was filtered, the filter cake was washed with DCM (1 mL×3). The filtrate was concentrated under reduced pressure, and added DCM (0.6 mL) to the concentrated filtrate. The system was purified by trituration with MTBE (1.8 mL). This resulted in 2-chloro-2-{4-cyano-3-[(fluorosulfonyl)oxy]phenyl}-3,3-dicyclohexyl-1,1-dimethyl-2H,3H-3lambda5-benzo[d]1-aza-3lambda5-phospha-2-auracyclopentan-1-ium-3-ylium-2,2-diuide (120 mg, 40.25% yield, 98.7% purity) as a grey solid. LCMS:(ES, m/z): [M]⁺=749.0.

¹H NMR (300 MHz, DMSO-d₆, ppm) δ 8.39 (dd, J=8.6, 4.4 Hz, 1H), 8.33 (s, 1H), 8.20 (d, J=8.2 Hz, 1H), 8.11-8.01 (m, 2H), 7.87 (dd, J=8.2, 1.3 Hz, 1H), 7.81 (td, J=7.5, 2.5 Hz, 1H), 3.51 (s, 6H), 2.94 (s, 2H), 1.94 (s, 2H), 1.82-1.52 (m, 10H), 1.29 (d, J=25.8 Hz, 4H), 1.07 (d, J=13.2 Hz, 2H), 0.60 (s, 2H)

### Example 4. Synthesis of Au-3-1-OMe

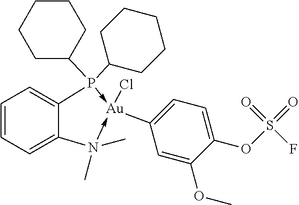

Step 1: To a stirred solution of 4-iodo-2-methoxyphenol (500 mg, 2.000 mmol, 1 equiv) and 3-(fluorosulfonyl)-1,2-dimethylimidazol-1-ium triflate (984.61 mg, 3.000 mmol, 1.5 equiv) in MeCN (5 mL) was added TEA (404.72 mg, 4.000 mmol, 2 equiv) dropwise at 0° C. under nitrogen atmosphere. The resulting mixture was concentrated under reduced pressure. The residue was purified by reversed-phase flash chromatography with the following conditions: column, C18 silica gel; mobile phase, MeCN in Water (0.1% FA), 10% to 95% gradient in 10 min; detector, UV 254 nm. This resulted in 4-iodo-2-methoxyphenyl sulfurofluoridate (300 mg, 45.18% yield) as a light yellow solid.

Step 2: To a stirred solution of Silver Hexafluoroantimonate(V) (103.47 mg, 0.301 mmol, 1 equiv) in DCM (3 mL) at 0° C. under nitrogen atmosphere. To the above mixture was added 4-iodo-2-methoxyphenyl sulfurofluoridate (100 mg, 0.301 mmol, 1 equiv) and 2-[(chloroaurio)dicyclohexyl-lambda5-phosphanyl]-N,N-dimethylaniline (165.58 mg, 0.301 mmol, 1 equiv) in portions at 0° C. The resulting mixture was stirred at room temperature for additional 3 h. The resulting mixture was filtered, the filter cake was washed with DCM (1 mL×3). The filtrate was concentrated under reduced pressure, and added DCM (0.6 mL) to the concentrated filtrate. The system was purified by trituration with MTBE (1.8 mL). This resulted in 2-chloro-3,3-dicyclohexyl-2-{4-[(fluorosulfonyl)oxy]-3-methoxyphenyl}-1,1-dimethyl-2H,3H-3lambda5-benzo[d]1-aza-3lambda5-phospha-2-auracyclopentan-1-ium-3-ylium-2,2-diuide (60 mg, 26.39% yield, 98.5% purity) as a grey solid. LCMS:(ES, m/z): [M]⁺=754.1.

¹H NMR (300 MHz, DMSO-d₆, ppm) δ 8.37 (dd, J=8.5, 4.3 Hz, 1H), 8.06 (q, J=7.9, 7.2 Hz, 2H), 7.79 (td, J=7.5, 2.3 Hz, 1H), 7.66 (d, J=8.5 Hz, 1H), 7.57 (s, 1H), 7.17 (dd, J=8.6, 1.7 Hz, 1H), 3.99 (s, 3H), 3.47 (s, 6H), 2.99 (s, 2H), 1.93 (s, 2H), 1.79-1.50 (m, 10H), 1.45-1.21 (m, 4H), 1.01 (s, 2H), 0.52 (s, 2H).

### Example 5. Synthesis of Au-3-2-CN

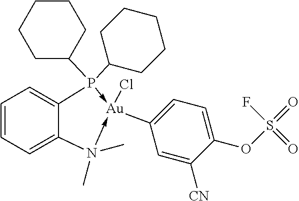

Step 1: To a stirred solution of 2-hydroxy-5-iodobenzonitrile (500 mg, 2.041 mmol, 1 equiv) and 3-(fluorosulfonyl)-1,2-dimethylimidazol-1-ium triflate (1004.77 mg, 3.061 mmol, 1.5 equiv) in MeCN (5 mL) was added TEA (413.00 mg, 4.082 mmol, 2 equiv) dropwise at 0° C. under nitrogen atmosphere. The resulting mixture was stirred at room temperature for additional 30 min. The resulting mixture was concentrated under reduced pressure. The residue was purified by reversed-phase flash chromatography with the following conditions: column, C18 silica gel; mobile phase, MeCN in Water (0.1% FA), 10% to 95% gradient in 10 min; detector, UV 254 nm. This resulted in 2-cyano-4-iodophenyl sulfurofluoridate (300 mg, 44.95% yield) as a light yellow oil.

Step 2: To a stirred solution of Silver Hexafluoroantimonate(V) (105.06 mg, 0.306 mmol, 1 equiv) in DCM (3 mL) at 0° C. under nitrogen atmosphere. To the above mixture was added 2-cyano-4-iodophenyl sulfurofluoridate (100 mg, 0.306 mmol, 1 equiv) and 2-[(chloroaurio)dicyclohexyl-lambda5-phosphanyl]-N,N-dimethylaniline (168.12 mg, 0.306 mmol, 1 equiv) dropwise at 0° C. The resulting mixture was stirred at 40° C. for additional overnight. The mixture was allowed to cool down to room temperature. The resulting mixture was filtered, the filter cake was washed with DCM (1 mL×3). The filtrate was concentrated under reduced pressure, and added DCM (0.6 mL) to the concentrated filtrate. The system was purified by trituration with MTBE (1.8 mL). This resulted in 2-chloro-2-{3-cyano-4-[(fluorosulfonyl)oxy]phenyl}-3,3-dicyclohexyl-1,1-dimethyl-2H,3H-3lambda5-benzo[d]1-aza-3lambda5-phospha-2-auracyclopentan-1-ium-3-ylium-2,2-diuide (60 mg, 26.16% yield, 91.5% purity) as a grey solid. LCMS:(ES, m/z): [M]⁺=749.1.

¹H NMR (300 MHz, DMSO-d₆, ppm) δ 8.44 (d, J=1.9 Hz, 1H), 8.39 (dd, J=8.5, 4.4 Hz, 1H), 8.15-7.99 (m, 4H), 7.80 (td, J=7.5, 2.5 Hz, 1H), 3.50 (s, 6H), 2.98 (s, 2H), 1.96 (s, 2H), 1.77-1.53 (m, 10H), 1.34 (s, 4H), 1.04 (t, J=12.8 Hz, 2H), 0.49 (s, 2H).

### Example 6. Synthesis of Au-MP-7

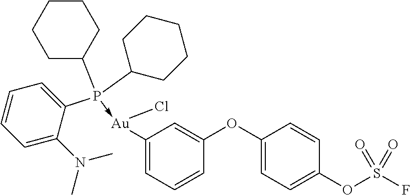

Step 1: To a stirred solution of 3-iodophenol (3 g, 13.636 mmol, 1 equiv) and 2-[4-(benzyloxy)phenyl]-4,4,5,5-tetramethyl-1,3,2-dioxaborolane (5.08 g, 16.363 mmol, 1.2 equiv) in 1,4-dioxane (5 mL) were added Cu(NO₃)₂ (3.84 g, 20.454 mmol, 1.5 equiv) and TMEDA (4.75 g, 40.908 mmol, 3 equiv) in portions at room temperature. The reaction was monitored by LCMS. The resulting mixture was filtered, the filter cake was washed with MeOH (3×1 mL). The filtrate was concentrated under reduced pressure. The residue product was purified by reverse phase flash to afford 1-[4-(benzyloxy)phenoxy]-3-iodobenzene (240 mg, 4.38% yield) as a white solid.

Step 2: To a stirred solution of 1-[4-(benzyloxy)phenoxy]-3-iodobenzene (240 mg, 0.597 mmol, 1 equiv) in DCM (3 mL) was added BBr₃ (3 mL, 0.448 mmol, 1.00 equiv) dropwise at 0° C. The reaction was monitored by LCMS. The reaction was quenched with ice water at 0° C. The resulting mixture was extracted with CH₂Cl₂ (3×1 mL). The combined organic layers were washed with water (3×1 mL), dried over anhydrous Na₂SO₄. After filtration, the filtrate was concentrated under reduced pressure. The residue was purified by silica gel column chromatography, eluted with PE/EA (5:1) to afford 4-(3-iodophenoxy)phenol (140 mg, 75.18% yield) as a yellow oil.

Step 3: To a stirred solution of 4-(3-iodophenoxy) phenol (140 mg, 0.449 mmol, 1 equiv) in ACN (3 mL) was added TEA (90.78 mg, 0.898 mmol, 2 equiv) dropwise at 0° C. The reaction was monitored by LCMS. The resulting mixture was concentrated under reduced pressure. The residue product was purified by reverse phase flash to afford 4-(3-iodophenoxy) phenyl sulfurofluoridate (80 mg, 45.25% yield) as a yellow solid.

Step 4: Into a 40 mL sealed tube were added Silver Hexafluoroantimonate(V) (69.74 mg, 0.203 mmol, 1 equiv) and DCM (10 mL) at 0° C. To the above mixture was added 2-[(chloroaurio)dicyclohexyl-lambda5-phosphanyl]-N,N-dimethylaniline (111.61 mg, 0.203 mmol, 1 equiv) and 4-(3-iodophenoxy)phenyl sulfurofluoridate (80 mg, 0.203 mmol, 1.00 equiv) dropwise over 3 min at 0° C. The resulting mixture was stirred at room temperature for additional overnight. The resulting mixture was filtered, the filter cake was washed with MeCN (3×3 mL). The filtrate was concentrated under reduced pressure, and added DCM (0.6 mL) to the concentrated filtrate. The system was purified by trituration with MTBE (1.8 mL). This resulted in 4-(3-{2-chloro-3,3-dicyclohexyl-1,1-dimethylbenzo[d]1-aza-3-phospha-2-auracyclopentan-2-yl}phenoxy) phenyl sulfurofluoridate (100 mg, 60.30% yield, 96.3% purity) as a white solid. LCMS:(ES, m/z): [M]+=816.1.

¹H NMR (300 MHz, DMSO-d₆, ppm) δ 8.34 (dd, J=8.6, 4.3 Hz, 1H), 8.11-7.98 (m, 2H), 7.78 (td, J=7.5, 2.4 Hz, 1H), 7.74-7.66 (m, 2H), 7.49 (t, J=7.9 Hz, 1H), 7.32 (d, J=8.0 Hz, 1H), 7.26-7.12 (m, 4H), 3.43 (s, 6H), 2.86 (s, 2H), 1.89 (s, 2H), 1.77-1.49 (m, 10H), 1.21 (d, J=17.6 Hz, 4H), 0.99 (s, 2H), 0.64 (s, 2H).

### Example 7. Synthesis of Au-PM-2

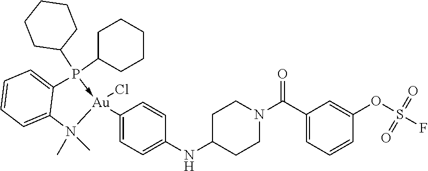

Step 1: To a stirred solution of benzenamine, 4-iodo- (500 mg, 2.283 mmol, 1 equiv), tert-butyl 4-oxopiperidine-1-carboxylate (454.86 mg, 2.283 mmol, 1 equiv) and AcOH (137.09 mg, 2.283 mmol, 1 equiv) in DCM (15 mL) at room temperature under nitrogen atmosphere. The mixture was stirred for 15 min. STAB (967.65 mg, 4.566 mmol, 2 equiv) was added and stirred for 2 h. The resulting mixture was diluted with water (10 mL) and extracted with DCM (15 mL×2). The mixture was acidified to pH 7 with saturated NaHCO₃ (aq.). The aqueous phase was extracted with EtOAc (10 mL×2). The combined organic layers were washed with brine (15 mL), dried over anhydrous Na₂SO₄. After filtration, the filtrate was concentrated under reduced pressure. The residue was purified by silica gel column chromatography, eluted with PE/THF (3:1) to afford tert-butyl 4-[(4-iodophenyl) amino]piperidine-1-carboxylate (750 mg, 81.67% yield) as a yellow solid.

Step 2: To a stirred solution of tert-butyl 4-[(4-iodophenyl) amino]piperidine-1-carboxylate (500 mg, 1.243 mmol, 1 equiv) in DCM (3 mL) and HCl in 1,4-dioxane (3 mL) at room temperature under nitrogen atmosphere. The resulting mixture was stirred at room temperature for additional 1 h. The resulting mixture was filtered, the filter cake was washed with DCM (3×3 mL). The filtrate was concentrated under reduced pressure. This resulted in N-(4-iodophenyl) piperidin-4-amine hydrochloride (350 mg, 83.16% yield) as a light yellow solid. The crude product mixture was used in the next step directly without further purification.

Step 3: To a stirred solution of N-(4-iodophenyl) piperidin-4-amine (300 mg, 0.993 mmol, 1 equiv), HATU (566.28 mg, 1.490 mmol, 1.5 equiv) and DIEA (192.49 mg, 1.490 mmol, 1.5 equiv) in DCM (5 mL) at room temperature under nitrogen atmosphere. The resulting mixture was stirred for 15 min. 3-[(fluorosulfonyl)oxy]benzoic acid (262.32 mg, 1.192 mmol, 1.2 equiv) was added and stirred for 30 min. The resulting mixture was diluted with H₂O (5 mL) and extracted with DCM (3×5 mL). The combined organic layers were washed with brine (5 mL), dried over anhydrous Na₂SO₄. The resulting mixture was concentrated under reduced pressure. The residue was purified by reversed-phase flash chromatography with the following conditions: column, C18 silica gel; mobile phase, MeCN in Water (0.1% FA), 10% to 50% gradient in 10 min; detector, UV 254 nm. This resulted in 3-{4-[(4-iodophenyl) amino]piperidine-1-carbonyl}phenyl sulfurofluoridate (250 mg, 49.93% yield) as a white solid.

Step 4: To a stirred solution of 3-{4-[(4-iodophenyl) amino]piperidine-1-carbonyl}phenyl sulfurofluoridate (100 mg, 0.198 mmol, 1 equiv) and 2-[(chloroaurio)dicyclohexyl-lambda5-phosphanyl]-N, N-dimethylaniline (109.03 mg, 0.198 mmol, 1 equiv) in DCM (5 mL) was added Silver Hexafluoroantimonate(V) (68.14 mg, 0.198 mmol, 1 equiv) dropwise at −20° C. The resulting mixture was stirred at −10° C. for 2 h. The reaction was monitored by LCMS. The resulting mixture was filtered, the filter cake was washed with DCM (5 mL) (3×1 mL). The filtrate was concentrated under reduced pressure, and added DCM (0.6 mL) to the concentrated filtrate. The system was purified by trituration with MTBE (1.8 mL). This resulted in 2-chloro-3,3-dicyclohexyl-2-{4-[(1-{3-[(fluorosulfonyl)oxy]benzoyl}piperidin-4-yl) amino]phenyl}-1,1-dimethyl-2H,3H-3lambda5-benzo[d]1-aza-3lambda5-phospha-2-auracyclopentan-1-ium-3-ylium-2,2-diuide (41 mg, 22.30% yield, 98.36% purity) as a brown solid. LCMS:(ES, m/z): [M]+=926.2.

¹H NMR (300 MHz, DMSO-d₆, ppm) δ 8.32 (dd, J=8.5, 4.4 Hz, 1H), 8.11-7.96 (m, 2H), 7.77 (t, J=7.8 Hz, 1H), 7.73-7.64 (m, 3H), 7.56 (dt, J=6.1, 2.2 Hz, 1H), 7.01 (d, J=8.4 Hz, 2H), 6.68 (d, J=8.5 Hz, 2H), 5.70 (s, 1H), 4.36 (s, 1H), 3.53 (s, 7H), 3.16 (d, J=36.4 Hz, 2H), 2.90 (d, J=11.5 Hz, 2H), 1.93 (d, J=57.6 Hz, 4H), 1.66 (d, J=25.4 Hz, 10H), 1.35 (s, 7H), 1.03 (d, J=13.6 Hz, 2H), 0.66 (s, 2H).

### Example 8. Synthesis of Au—PP-10

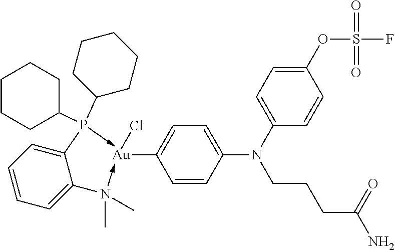

Step 1: To a stirred mixture of P-anisidine (2 g, 16.240 mmol, 1 equiv) and 1,4-diiodobenzene (8.04 g, 24.360 mmol, 1.5 equiv) in toluene (40 mL) were added t-BuONa (4.68 g, 48.720 mmol, 3 equiv) and Pd(dppf)Cl₂ (2.38 g, 3.248 mmol, 0.2 equiv) in portions at room temperature under nitrogen atmosphere. The resulting mixture was stirred at 70° C. for 2 h under nitrogen atmosphere. The resulting mixture was extracted with EtOAc (3×40 mL). The combined organic layers were washed with water (3×40 mL), dried over anhydrous Na₂SO₄. After filtration, the filtrate was concentrated under reduced pressure. The residue was purified by reversed-phase flash chromatography with the following conditions: column, C18 silica gel; mobile phase, MeCN in Water (0.1% FA), 10% to 95% gradient in 10 min; detector, UV 254 nm. This resulted in N-(4-iodophenyl)-4-methoxyaniline (2 g, 37.88% yield) as a brown oil.

Step 2: A mixture of N-(4-iodophenyl)-4-methoxyaniline (1.5 g, 4.613 mmol, 1 equiv) and methyl 4-chloro-4-oxobutanoate (0.83 g, 5.536 mmol, 1.2 equiv) in toluene (20 mL) was stirred at 90° C. for 2 h. The resulting mixture was extracted with EtOAc (3×20 mL). The combined organic layers were washed with water (3×10 mL), dried over anhydrous Na₂SO₄. After filtration, the filtrate was concentrated under reduced pressure. The residue was purified by reversed-phase flash chromatography with the following conditions: column, C18 silica gel; mobile phase, MeCN in Water (0.1% NH3·H2O), 10% to 95% gradient in 10 min; detector, UV 254 nm. This resulted in methyl 3-[(4-iodophenyl)(4-methoxyphenyl)carbamoyl]propanoate (1 g, 49.35% yield) as a brown oil.

Step 3: A solution of methyl 3-[(4-iodophenyl) (4-methoxyphenyl) carbamoyl]propanoate (1 g, 2.277 mmol, 1 equiv) in Borane-tetrahydrofuran complex (1.0 M in THF) (15 mL) was stirred at 0° C. for 12 h. The reaction was quenched by the addition of MeOH (3 mL) at 0° C. The resulting mixture was concentrated under reduced pressure. The residue was purified by reversed-phase flash chromatography with the following conditions: column, C18 silica gel; mobile phase, MeCN in Water (0.1% NH3·H2O), 10% to 95% gradient in 15 min; detector, UV 254 nm. This resulted in methyl 4-[(4-hydroxyphenyl) (4-iodophenyl) amino]butanoate (500 mg, 53.41% yield) as a yellow oil.

Step 4: A solution of methyl 4-[(4-iodophenyl) (4-methoxyphenyl) amino]butanoate (500 mg, 1.176 mmol, 1 equiv) in DCM (2 mL) and BBr₃ (4 mL) was stirred at 0° C. for 2 h. The reaction was quenched by the addition of ice water (20 mL) at 0° C. The resulting mixture was extracted with CH₂Cl₂ (3×20 mL). The combined organic layers were washed with water (3×20 mL), dried over anhydrous Na₂SO₄. After filtration, the filtrate was concentrated under reduced pressure. The resulting mixture was concentrated under reduced pressure. The residue was purified by reversed-phase flash chromatography with the following conditions: column, C18 silica gel; mobile phase, MeCN in Water (0.1% NH3·H2O), 10% to 95% gradient in 10 min; detector, UV 254 nm. This resulted in methyl 4-[(4-hydroxyphenyl)(4-iodophenyl)amino]butanoate (300 mg, 62.05% yield) as a yellow oil.

Step 5: A solution of methyl 4-[(4-hydroxyphenyl)(4-iodophenyl)amino]butanoate (300 mg, 0.730 mmol, 1 equiv) in NH3·H2O (5 mL) was stirred at room temperature for 12 h. The residue was purified by reversed-phase flash chromatography with the following conditions: column, C18 silica gel; mobile phase, MeCN in Water (0.1% NH3·H2O), 10% to 95% gradient in 10 min; detector, UV 254 nm. This resulted in 4-[(4-hydroxyphenyl) (4-iodophenyl) amino]butanamide (60 mg, 20.76% yield) as a yellow oil.

Step 6: To a stirred mixture of 4-[(4-hydroxyphenyl) (4-iodophenyl) amino]butanamide (50 mg, 0.126 mmol, 1 equiv) and 3-(fluorosulfonyl)-1,2-dimethylimidazol-1-ium triflate (49.71 mg, 0.151 mmol, 1.2 equiv) in ACN (3 mL) was added TEA (19.15 mg, 0.189 mmol, 1.5 equiv) in portions at 0° C. The resulting mixture was stirred at 0° C. for 30 min. The resulting mixture was concentrated under reduced pressure. The residue was purified by reversed-phase flash chromatography with the following conditions: column, C18 silica gel; mobile phase, MeCN in Water (0.1% NH₃·H₂O), 10% to 50% gradient in 10 min; detector, UV 254 nm. This resulted in 4-[(3-carbamoylpropyl) (4-iodophenyl) amino]phenyl sulfurofluoridate (40 mg, 66.28% yield) as a colorless oil.

Step 7: To a stirred solution of 4-[(3-carbamoylpropyl) (4-iodophenyl) amino]phenyl sulfurofluoridate (30 mg, 0.063 mmol, 1 equiv) and 2-[(chloroaurio)dicyclohexyl-lambda5-phosphanyl]-N,N-dimethylaniline (34.49 mg, 0.063 mmol, 1 equiv) in DCM (3 mL) were added Silver Hexafluoroantimonate(V) (21.55 mg, 0.063 mmol, 1 equiv) in portions at 0° C. under nitrogen atmosphere. The resulting mixture was stirred at room temperature for additional 2 h. The resulting mixture was filtered, the filter cake was washed with DCM (3×3 mL). The filtrate was concentrated under reduced pressure, and added DCM (0.6 mL) to the concentrated filtrate. The system was purified by trituration with MTBE (1.8 mL). The precipitated solids were collected by filtration and washed with MTBE (1 mL) to afford in 2-{4-[(3-carbamoylpropyl) ({4-[(fluorosulfonyl)oxy]phenyl}) amino]phenyl}-2-chloro-3,3-dicyclohexyl-1,1-dimethyl-2H,3H-3lambda5-benzo[d]1-aza-3lambda5-phospha-2-auracyclopentan-1-ium-3-ylium-2,2-diuide (29 mg, 51.30% yield, 96.3% purity) as a light green solid. LCMS:(ES, m/z): [M]+=900.2 ¹H NMR (300 MHz, DMSO-d₆, ppm) δ 8.36 (dd, J=8.4, 4.3 Hz, 1H), 8.13-7.98 (m, 2H), 7.79 (t, J=7.7 Hz, 1H), 7.41 (dd, J=8.8, 7.0 Hz, 4H), 7.31 (s, 1H), 7.23 (d, J=8.5 Hz, 2H), 6.99 (d, J=9.3 Hz, 2H), 6.79 (s, 1H), 3.74 (t, J=7.8 Hz, 2H), 3.44 (s, 6H), 3.00 (d, J=11.2 Hz, 2H), 2.15 (t, J=7.2 Hz, 2H), 1.89 (s, 2H), 1.81-1.44 (m, 12H), 1.36 (q, J=13.3 Hz, 4H), 1.01 (d, J=13.6 Hz, 2H), 0.54 (s, 2H).

### Example 9. Synthesis of SFY—Au-7-1

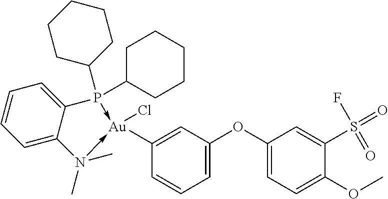

Step 1: To a stirred solution of 2-methoxy-5-(4,4,5,5-tetramethyl-1,3,2-dioxaborolan-2-yl) benzenesulfonyl fluoride (500 mg, 1.582 mmol, 1 equiv) and 3-iodophenol (347.95 mg, 1.582 mmol, 1 equiv) in dioxane (5 mL) were added Cu(NO₃)₂ (444.93 mg, 2.373 mmol, 1.5 equiv) and TMEDA (551.36 mg, 4.746 mmol, 3 equiv) dropwise at room temperature. The resulting mixture was stirred at 80° C. for 2 h. The reaction was monitored by LCMS. The resulting mixture was concentrated under reduced pressure. The residue product was purified by reverse phase flash to afford 5-(3-iodophenoxy)-2-methoxybenzenesulfonyl fluoride (100 mg, 15.49% yield) as a white solid.

Step 2: To a stirred solution of 5-(3-iodophenoxy)-2-methoxybenzenesulfonyl fluoride (100 mg, 0.245 mmol, 1 equiv) and 2-[(chloroaurio)dicyclohexyl-lambda5-phosphanyl]-N, N-dimethylaniline (134.71 mg, 0.245 mmol, 1 equiv) in DCM (5 mL) was added AgSbF₆ (84.18 mg, 0.245 mmol, 1 equiv) dropwise at −20° C. The resulting mixture was stirred at −10° C. for 2 h under nitrogen atmosphere. The reaction was monitored by LCMS. The resulting mixture was filtered, the filter cake was washed with DCM (3 mL) (3×1 mL). The filtrate was concentrated under reduced pressure, and added DCM (0.6 mL) to the concentrated filtrate. The system was purified by trituration with MTBE (1.8 mL). This resulted in 2-chloro-3,3-dicyclohexyl-2-{3-[3-(fluorosulfonyl)-4-methoxyphenoxy]phenyl}-1,1-dimethyl-2H,3H-3lambda5-benzo[d]1-aza-3lambda5-phospha-2-auracyclopentan-1-ium-3-ylium-2,2-diuide (129.3 mg, 63.50% yield, 98.6% purity) as a white solid. LCMS:(ES, m/z): [M]⁺=830.1.

¹H NMR (300 MHz, DMSO-d₆, ppm) δ 8.28 (dd, J=8.5, 4.4 Hz, 1H), 8.00 (d, J=8.8 Hz, 2H), 7.76 (dd, J=10.7, 4.2 Hz, 1H), 7.69 (dd, J=9.2, 2.9 Hz, 1H), 7.61-7.52 (m, 2H), 7.47 (t, J=8.0 Hz, 1H), 7.30 (d, J=8.0 Hz, 1H), 7.15 (d, J=8.0 Hz, 1H), 7.04 (s, 1H), 3.99 (s, 3H), 3.34 (d, J=45.4 Hz, 6H), 1.97-1.37 (s, 12H), 1.34-0.83 (m, 6H), 0.57 (s, 2H).

### Example 10. Synthesis of SFY—Au-8-1

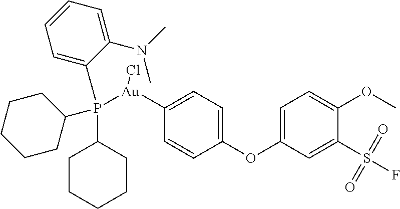

Step 1: To a stirred solution of 5-bromo-2-methoxybenzenesulfonyl fluoride (2.5 g, 9.291 mmol, 1 equiv) and bis(pinacolato)diboron (3.54 g, 13.937 mmol, 1.5 equiv) in DMSO (20 mL) were added Pd(dppf)Cl₂ (0.68 g, 0.929 mmol, 0.1 equiv) and K₂CO₃ (3.85 g, 27.873 mmol, 3 equiv) dropwise at room temperature. The resulting mixture was stirred at 90° C. for 3 h under nitrogen atmosphere. The resulting mixture was extracted with EtOAc (3×3 mL). The combined organic layers were washed with water (3×3 mL), dried over anhydrous Na₂SO₄. After filtration, the filtrate was concentrated under reduced pressure. The residue was purified by silica gel column chromatography, eluted with PE/EA (5:1) to afford 2-methoxy-5-(4,4,5,5-tetramethyl-1,3,2-dioxaborolan-2-yl)benzenesulfonyl fluoride (2 g, 68.09% yield) as a yellow oil.

Step 2: To a stirred solution of 2-methoxy-5-(4,4,5,5-tetramethyl-1,3,2-dioxaborolan-2-yl) benzenesulfonyl fluoride (500 mg, 1.582 mmol, 1 equiv) and 4-iodophenol (347.95 mg, 1.582 mmol, 1 equiv) in dioxane (5 mL) were added Cu(NO₃)₂ (444.93 mg, 2.373 mmol, 1.5 equiv) and TMEDA (551.36 mg, 4.746 mmol, 3 equiv) dropwise at room temperature. The resulting mixture was stirred at 80° C. for 2 h. The reaction was monitored by LCMS. The resulting mixture was concentrated under reduced pressure. The residue product was purified by reverse phase flash to afford 5-(4-iodophenoxy)-2-methoxybenzenesulfonyl fluoride (50 mg, 7.75% yield) as a white solid.

Step 3: To a stirred solution of 5-(4-iodophenoxy)-2-methoxybenzenesulfonyl fluoride (50 mg, 0.122 mmol, 1 equiv) and 2-[(chloroaurio)dicyclohexyl-lambda5-phosphanyl]-N, N-dimethylaniline (67.36 mg, 0.122 mmol, 1 equiv) in DCM (3 mL) was added Silver Hexafluoroantimonate(V) (42.09 mg, 0.122 mmol, 1 equiv) dropwise at −20° C. The resulting mixture was stirred at −10° C. for 2 h. The reaction was monitored by LCMS. The resulting mixture was filtered, the filter cake was washed with DCM (3 mL) (3×1 mL). The filtrate was concentrated under reduced pressure, and added DCM (0.6 mL) to the concentrated filtrate. The system was purified by trituration with MTBE (1.8 mL). This resulted in [(2-{[chloro({4-[3-(fluorosulfonyl)-4-methoxyphenoxy]phenyl}) aurio]dicyclohexyl-lambda5-phosphanyl}phenyl) dimethylammonio]methanidylidene (35.1 mg, 46.44% yield, 97.7% purity) as a white solid. LCMS:(ES, m/z): [M]⁺=830.1.

¹H NMR (300 MHz, DMSO-d₆, ppm) δ 8.35 (dd, J=8.7, 4.3 Hz, 1H), 8.05 (q, J=9.2 Hz, 2H), 7.79 (t, J=6.8 Hz, 1H), 7.67 (dd, J=9.1, 3.0 Hz, 1H), 7.55 (d, J=9.2 Hz, 1H), 7.51-7.25 (m, 3H), 7.17 (dd, J=8.5, 4.2 Hz, 2H), 4.02 (s, 3H), 3.44 (s, 6H), 2.94 (d, J=11.2 Hz, 2H), 1.88 (s, 2H), 1.79-1.47 (m, 10H), 1.41-1.24 (m, 4H), 1.02 (d, J=13.1 Hz, 2H), 0.59 (s, 2H).

### Example 11. Synthesis of Au-8-2

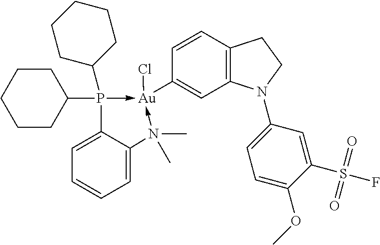

Step 1: To a stirred solution of 2-methoxy-5-(4,4,5,5-tetramethyl-1,3,2-dioxaborolan-2-yl) benzenesulfonyl fluoride (500 mg, 1.582 mmol, 1 equiv) and 5-iodo-2,3-dihydro-1H-indole (387.57 mg, 1.582 mmol, 1 equiv) in dioxane (10 mL) were added Cu(NO₃)₂ (444.93 mg, 2.373 mmol, 1.5 equiv) and TMEDA (551.36 mg, 4.746 mmol, 3 equiv) dropwise at room temperature. The resulting mixture was stirred at 80° C. for 2 h. The reaction was monitored by LCMS. The resulting mixture was concentrated under reduced pressure. The residue product was purified by reverse phase flash to afford 5-(6-iodo-2,3-dihydroindol-1-yl)-2-methoxybenzenesulfonyl fluoride (50 mg, 7.30% yield) as a white solid.

Step 2: To a stirred solution of 5-(6-iodo-2,3-dihydroindol-1-yl)-2-methoxybenzenesulfonyl fluoride (50 mg, 0.115 mmol, 1 equiv) and (chloroaurio)dicyclohexyl[2-(dimethylamino) phenyl]-lambda5-phosphanyl (63.46 mg, 0.115 mmol, 1 equiv) in DCM (3 mL) was added Silver Hexafluoroantimonate(V) (39.66 mg, 0.115 mmol, 1 equiv) dropwise at −20° C. The resulting mixture was stirred at −10° C. for 2 h under nitrogen atmosphere. The reaction was monitored by LCMS. The resulting mixture was filtered, the filter cake was washed with DCM (3 mL) (3×1 mL). The filtrate was concentrated under reduced pressure, and added DCM (0.6 mL) to the concentrated filtrate. The system was purified by trituration with MTBE (1.8 mL). This resulted in [chloro({1-[3-(fluorosulfonyl)-4-methoxyphenyl]-2,3-dihydroindol-6-yl}) aurio]dicyclohexyl[2-(dimethylamino) phenyl]-lambda5-phosphanyl (25.7 mg, 26.01% yield, 87.8% purity) as a white solid. LCMS:(ES, m/z): [M]⁺=855.1.

¹H NMR (300 MHz, DMSO-d₆, ppm) δ 8.34 (dd, J=8.5, 4.3 Hz, 1H), 8.04 (q, J=9.2 Hz, 2H), 7.86-7.73 (m, 2H), 7.68 (d, J=2.8 Hz, 1H), 7.50 (d, J=9.2 Hz, 1H), 7.25 (s, 1H), 7.04 (s, 2H), 4.01 (d, J=7.8 Hz, 5H), 3.41 (s, 6H), 3.19 (t, J=8.3 Hz, 2H), 2.91 (d, J=11.3 Hz, 2H), 1.87 (s, 2H), 1.80-1.48 (m, 10H), 1.41-1.25 (m, 4H), 1.06 (d, J=12.6 Hz, 2H), 0.70 (s, 2H).

### Example 12. Synthesis of Au-1-1-OMe

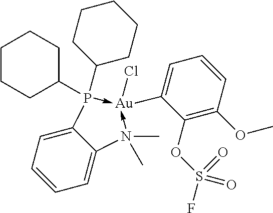

Step 1: To a stirred mixture of 2-iodo-6-methoxyphenol (250 mg, 1.000 mmol, 1.00 equiv) and 3-(fluorosulfonyl)-1,2-dimethylimidazol-1-ium triflate (393.84 mg, 1.200 mmol, 1.20 equiv) in ACN (6 mL) was added TEA (202.36 mg, 2.000 mmol, 2 equiv) dropwise at 0° C. The resulting mixture was stirred at room temperature for additional 30 min. The reaction was monitored by LCMS. The residue was purified by reversed-phase flash chromatography with the following conditions: column, C18 silica gel; mobile phase, MeCN in Water (0.1% FA), 10% to 60% gradient in 10 min; detector, UV 254 nm. This resulted in 2-iodo-6-methoxyphenyl sulfurofluoridate (130 mg, 39.15% yield) as a white solid.

Step 2: To a stirred mixture of AgSbF₆ (124.17 mg, 0.361 mmol, 1 equiv) in DCM (6 mL) were added 2-iodo-6-methoxyphenyl sulfurofluoridate (120 mg, 0.361 mmol, 1 equiv) and (chloroaurio)dicyclohexyl[2-(dimethylamino) phenyl]-lambda5-phosphanyl (198.70 mg, 0.361 mmol, 1 equiv) dropwise at 0° C. The resulting mixture was stirred at 25° C. for additional 2 h. The resulting mixture was filtered, and the filter cake was washed with DCM. The filtrate was concentrated under reduced pressure, and added DCM (1 mL) to the concentrated filtrate. The system was purified by trituration with MTBE (3 mL). This resulted in 2-{2-chloro-3,3-dicyclohexyl-1,1-dimethylbenzo[d]1-aza-3-phospha-2-auracyclopentan-2-yl}-6-methoxyphenyl sulfurofluoridate (120 mg, 43.98% yield, 97.51% purity) as a light grey solid. LCMS: (ES, m/z): [M]⁺=754.0.

¹H NMR (300 MHz, DMSO-d₆, ppm) δ 8.40 (dd, J=8.6, 4.4 Hz, 1H), 8.17-7.96 (m, 2H), 7.82 (td, J=7.4, 2.5 Hz, 1H), 7.50 (t, J=8.1 Hz, 1H), 7.32 (ddd, J=22.9, 8.2, 1.3 Hz, 2H), 3.97 (s, 3H), 3.58 (s, 6H), 2.95 (dd, J=67.6, 10.5 Hz, 2H), 1.90 (s, 2H), 1.82-1.53 (m, 8H), 1.51-1.16 (m, 6H), 1.06 (dd, J=23.5, 10.3 Hz, 2H), 0.70-0.49 (m, 2H).

### Example 13. Synthesis of Au-2-1-OMe

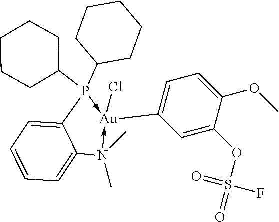

Step 1: To a stirred solution of 5-iodo-2-methoxyphenol (250 mg, 1.000 mmol, 1 equiv) and 3-(fluorosulfonyl)-1,2-dimethylimidazol-1-ium triflate (492.31 mg, 1.500 mmol, 1.5 equiv) in MeCN (3 mL) was added TEA (202.36 mg, 2.000 mmol, 2 equiv) dropwise at 0° C. under nitrogen atmosphere. The resulting mixture was stirred at room temperature for additional 30 min. The resulting mixture was concentrated under reduced pressure. The residue was purified by reversed-phase flash chromatography with the following conditions: column, C18 silica gel; mobile phase, MeCN in Water (0.1% FA), 10% to 95% gradient in 10 min; detector, UV 254 nm. This resulted in 5-iodo-2-methoxyphenyl sulfurofluoridate (120 mg, 36.14% yield) as a yellow oil.

Step 2: To a stirred solution of Silver Hexafluoroantimonate(V) (124.17 mg, 0.361 mmol, 1 equiv) in DCM (3 mL) at 0° C. under nitrogen atmosphere. To the above mixture was added 5-iodo-2-methoxyphenyl sulfurofluoridate (120 mg, 0.361 mmol, 1 equiv) and 2-[(chloroaurio)dicyclohexyl-lambda5-phosphanyl]-N,N-dimethylaniline (198.70 mg, 0.361 mmol, 1 equiv) in portions at 0° C. The resulting mixture was stirred at room temperature for additional 3 h. The resulting mixture was filtered, the filter cake was washed with DCM (1 mL×3). The filtrate was concentrated under reduced pressure, and added DCM (0.6 mL) to the concentrated filtrate. The system was purified by trituration with MTBE (1.8 mL). This resulted in 2-chloro-3,3-dicyclohexyl-2-{3-[(fluorosulfonyl)oxy]-4-methoxyphenyl}-1,1-dimethyl-2H,3H-3lambda5-benzo[d]1-aza-3lambda5-phospha-2-auracyclopentan-1-ium-3-ylium-2,2-diuide (60 mg, 21.99% yield, 96% purity) as a grey solid. LCMS:(ES, m/z): [M]⁺=754.1.

¹H NMR (300 MHz, DMSO-d₆, ppm) δ 8.39 (dd, J=8.5, 4.5 Hz, 1H), 8.09 (d, J=7.6 Hz, 3H), 7.83 (dt, J=8.4, 4.2 Hz, 1H), 7.68 (dd, J=8.6, 1.8 Hz, 1H), 7.59 (d, J=8.9 Hz, 1H), 3.99 (s, 3H), 3.33 (s, 6H), 3.12-2.80 (m, 2H), 2.15 (s, 1H), 1.92-1.59 (m, 11H), 1.33 (s, 4H), 1.09 (d, J=10.7 Hz, 2H), 0.66 (s, 2H).

### Example 14. Synthesis of Au-5-1-Me

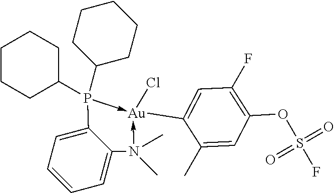

Step 1: To a stirred solution of 2-fluoro-4-iodo-5-methylphenol (200 mg, 0.794 mmol, 1 equiv) and 3-(fluorosulfonyl)-1,2-dimethylimidazol-1-ium triflate (312.59 mg, 0.953 mmol, 1.2 equiv) in ACN (8 mL) was added TEA (120.45 mg, 1.191 mmol, 1.5 equiv) dropwise at 0° C. The resulting mixture was stirred at room temperature for 20 min. The resulting mixture was concentrated under reduced pressure. The residue was purified by reversed-phase flash chromatography with the following conditions: column, C18 silica gel; mobile phase, MeCN in Water (0.1% FA), 10% to 50% gradient in 10 min; detector, UV 254 nm. This resulted in 2-fluoro-4-iodo-5-methylphenyl sulfurofluoridate (72 mg, 27.16% yield) as a yellow oil.

Step 2: To a stirred solution of 2-fluoro-4-iodo-5-methylphenyl sulfurofluoridate (50 mg, 0.150 mmol, 1 equiv) and 2-[(chloroaurio)dicyclohexyl-lambda5-phosphanyl]-N, N-dimethylaniline (82.30 mg, 0.150 mmol, 1 equiv) in DCM (2 mL) was added AgSbF₆ (51.43 mg, 0.150 mmol, 1 equiv) dropwise at 0° C. The resulting mixture was stirred at room temperature for overnight. The resulting mixture was filtered, the filter cake was washed with DCM (2 mL). The filtrate was concentrated under reduced pressure, and added DCM (1 mL) to the concentrated filtrate. The system was purified by trituration with MTBE (4 mL). This resulted in 4-{2-chloro-3,3-dicyclohexyl-1,1-dimethylbenzo[d]1-aza-3-phospha-2-auracyclopentan-2-yl}-2-fluoro-5-methylphenyl sulfurofluoridate (80 mg, 70.61% yield, 100.0% purity) as a grey solid. LCMS:(ES, m/z): [M]⁺=756.1.

¹H NMR (300 MHz, DMSO-d₆, ppm) δ 8.38 (dd, J=8.6, 4.4 Hz, 1H), 8.05 (td, J=7.7, 2.1 Hz, 2H), 7.81 (dd, J=7.7, 4.0 Hz, 2H), 7.70 (d, J=10.0 Hz, 1H), 3.49 (d, J=9.0 Hz, 6H), 3.12 (d, J=10.7 Hz, 1H), 2.95 (d, J=11.1 Hz, 1H), 2.58 (s, 3H), 2.17 (s, 1H), 1.90-1.49 (m, 10H), 1.48-1.14 (m, 5H), 1.04 (d, J=13.3 Hz, 2H), 0.71 (s, 1H), 0.41 (d, J=8.5 Hz, 1H).

### Example 15. Synthesis of Au-6

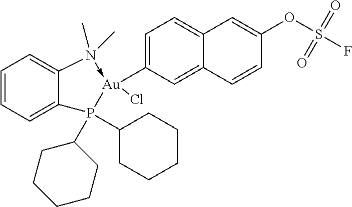

Step 1: To a stirred mixture of 6-bromo-2-naphthol (1 g, 4.483 mmol, 1 equiv) and KI (3.72 g, 22.415 mmol, 5 equiv) in DMF (15 mL) were added NiBr₂ (0.29 g, 1.345 mmol, 0.3 equiv) and Tributylphosphane (0.27 g, 1.345 mmol, 0.3 equiv) in portions at room temperature under nitrogen atmosphere. The resulting mixture was stirred at 120° C. for 12 h under nitrogen atmosphere. The resulting mixture was filtered, the filter cake was washed with MeOH (3×3 mL). The filtrate was concentrated under reduced pressure. The residue was purified by reversed-phase flash chromatography with the following conditions: column, C18 silica gel; mobile phase, MeCN in Water (0.1% FA), 10% to 95% gradient in 10 min; detector, UV 254 nm. This resulted in 6-iodonaphthalen-2-ol (600 mg, 49.56% yield) as a yellow solid.

Step 2: To a stirred mixture of 6-iodonaphthalen-2-ol (500 mg, 1.851 mmol, 1 equiv) and 3-(fluorosulfonyl)-1,2-dimethylimidazol-1-ium triflate (729.26 mg, 2.221 mmol, 1.2 equiv) in ACN (5 mL) were added TEA (281.02 mg, 2.776 mmol, 1.5 equiv) in portions at 0° C. The resulting mixture was stirred at 0° C. for 30 min. The resulting mixture was concentrated under reduced pressure. The residue was purified by reversed-phase flash chromatography with the following conditions: column, C18 silica gel; mobile phase, MeCN in Water (0.1% FA), 10% to 95% gradient in 10 min; detector, UV 254 nm. This resulted in 6-iodonaphthalen-2-yl sulfurofluoridate (300 mg, 46.02% yield) as a yellow solid.

Step 3: To a stirred solution of 6-bromonaphthalen-2-yl sulfurofluoridate (100 mg, 0.328 mmol, 1 equiv) and (chloroaurio)dicyclohexyl[2-(dimethylamino) phenyl]-lambda5-phosphanyl (180.21 mg, 0.328 mmol, 1 equiv) in DCM (3 mL) were added Silver Hexafluoroantimonate(V) (112.62 mg, 0.328 mmol, 1 equiv) in portions at 0° C. under nitrogen atmosphere. The resulting mixture was stirred at room temperature for additional 2 h. The reaction was monitored by LCMS. The resulting mixture was filtered, the filter cake was washed with DCM (3×3 mL). The filtrate was concentrated under reduced pressure, and added DCM (1 mL) to the concentrated filtrate. The system was purified by trituration with MTBE (3 mL). This resulted in [chloro({6-[(fluorosulfonyl) oxy]naphthalen-2-yl}) aurio]dicyclohexyl[2-(dimethylamino) phenyl]-lambda5-phosphanyl (62 mg, 24.41% yield, 95.6% purity) as a yellow solid. LCMS:(ES, m/z): [M]⁺=774.1.

¹H NMR (300 MHz, DMSO-d₆, ppm) δ 8.38 (dd, J=8.7, 4.3 Hz, 1H), 8.32 (d, J=2.6 Hz, 1H), 8.20 (d, J=9.1 Hz, 1H), 8.16-7.98 (m, 4H), 7.85-7.72 (m, 3H), 3.36 (s, 6H), 3.03-2.84 (m, 2H), 1.92 (s, 2H), 1.82-1.44 (m, 10H), 1.44-1.18 (m, 4H), 1.06 (t, J=13.0 Hz, 2H), 0.62 (s, 2H).

### Example 16. Synthesis of Au-7

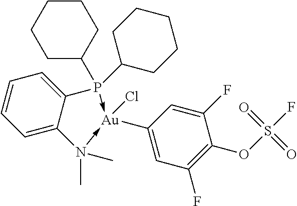

Step 1: To a stirred mixture of 2,6-difluoro-4-iodophenol (800 mg, 3.12 mmol, 1 equiv) and 3-(fluorosulfonyl)-1,2-dimethylimidazol-1-ium triflate (1538.73 mg, 4.68 mmol, 1.5 equiv) in ACN (3 mL) was added TEA (632.48 mg, 6.25 mmol, 2 equiv) dropwise at 0° C. The reaction was monitored by LCMS. The resulting mixture was concentrated under reduced pressure. The crude product was purified by reverse phase flash to afford 2,6-difluoro-4-iodophenyl sulfurofluoridate (120 mg, 11.36% yield) as a off-white oil.

Step 2: A solution of Silver Hexafluoroantimonate(V) (116.90 mg, 0.34 mmol, 1 equiv) in DCM (3 mL) was treated with 2,6-difluoro-4-iodophenyl sulfurofluoridate (115 mg, 0.34 mmol, 1 equiv) at 0° C. for 3 min under nitrogen atmosphere followed by the addition of 2-[(chloroaurio)dicyclohexyl-lambda5-phosphanyl]-N,N-dimethylaniline (187.41 mg, 0.34 mmol, 1 equiv) in portions at 0° C. The resulting mixture was filtered, the filter cake was washed with DCM (3 mL) (3×2 mL). The filtrate was concentrated under reduced pressure, and added DCM (1 mL) to the concentrated filtrate. The system was purified by trituration with MTBE (4 mL). This resulted in 4-{2-chloro-3,3-dicyclohexyl-1,1-dimethylbenzo[d]1-aza-3-phospha-2-auracyclopentan-2-yl}-2,6-difluorophenyl sulfurofluoridate (100 mg, 38.63% yield) as a grey solid. LCMS:(ES, m/z): [M]⁺=760.1.

¹H NMR (300 MHz, DMSO-d₆, ppm) δ 8.38 (dd, J=8.5, 4.3 Hz, 1H), 8.05 (t, J=9.3 Hz, 2H), 7.82 (d, J=8.9 Hz, 3H), 3.49 (s, 6H), 3.02 (d, J=11.2 Hz, 2H), 1.99 (s, 2H), 1.87-1.48 (m, 10H), 1.34 (d, J=12.9 Hz, 4H), 1.00 (d, J=12.9 Hz, 2H), 0.66-0.44 (s, 2H).

### Example 17. Synthesis of Au-8

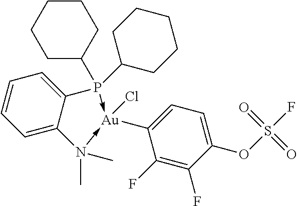

Step 1: To a stirred mixture of 2,3-difluoro-4-iodophenol (500 mg, 1.95 mmol, 1 equiv) and 3-(fluorosulfonyl)-1,2-dimethylimidazol-1-ium triflate (961.71 mg, 2.92 mmol, 1.5 equiv) in ACN (3 mL) was added TEA (395.30 mg, 3.90 mmol, 2 equiv) dropwise at 0° C. The reaction was monitored by LCMS. The resulting mixture was concentrated under reduced pressure. The crude product was purified by reverse phase flash to afford 2,3-difluoro-4-iodophenyl sulfurofluoridate (60 mg, 9.09% yield) as a yellow oil.

Step 2: To a stirred mixture of Silver Hexafluoroantimonate(V) (60.99 mg, 0.17 mmol, 1 equiv) in DCM (3 mL) were added 2,3-difluoro-4-iodophenyl sulfurofluoridate (60 mg, 0.17 mmol, 1 equiv) and 2-[(chloroaurio)dicyclohexyl-lambda5-phosphanyl]-N,N-dimethylaniline (97.78 mg, 0.17 mmol, 1 equiv) in portions at 0° C. under nitrogen atmosphere. The reaction was monitored by LCMS. The resulting mixture was filtered, the filter cake was washed with DCM (3 mL) (3×1 mL). The filtrate was concentrated under reduced pressure, and added DCM (1 mL) to the concentrated filtrate. The system was purified by trituration with MTBE (3 mL). This resulted in 4-{2-chloro-3,3-dicyclohexyl-1,1-dimethylbenzo[d]1-aza-3-phospha-2-auracyclopentan-2-yl}-2,3-difluorophenyl sulfurofluoridate (60 mg, 44.42% yield) as a grey solid. LCMS:(ES, m/z): [M]+=760.1.

¹H NMR (300 MHz, DMSO-d₆, ppm) δ 8.41 (dd, J=8.5, 4.3 Hz, 1H), 8.08 (t, J=9.1 Hz, 2H), 7.91-7.78 (m, 2H), 7.69 (d, J=9.2 Hz, 1H), 3.56 (d, J=13.3 Hz, 6H), 3.08 (s, 2H), 1.98 (d, J=37.0 Hz, 2H), 1.65 (s, 9H), 1.46-1.24 (m, 5H), 1.14-0.86 (m, 2H), 0.55 (s, 2H).

### Example 18. Synthesis of Au-MM-1

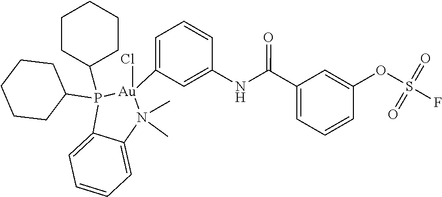

Step 1: To a stirred solution of 3-[(fluorosulfonyl)oxy]benzoic acid (331.73 mg, 1.507 mmol, 1.10 equiv), DIEA (531.09 mg, 4.110 mmol, 3 equiv) and HATU (781.22 mg, 2.055 mmol, 1.5 equiv) in DCM (8 mL) were added 3-iodo-benzenamine (300 mg, 1.370 mmol, 1.00 equiv) dropwise at 0° C. The resulting mixture was stirred at 25° C. for additional 1 h. The reaction was monitored by LCMS. The resulting mixture was extracted with DCM (3×5 mL). The combined organic layers were dried over anhydrous Na₂SO₄. After filtration, the filtrate was concentrated under reduced pressure. The residue was purified by reversed-phase flash chromatography with the following conditions: column, C18 silica gel; mobile phase, MeCN in Water (0.1% FA), 10% to 70% gradient in 12 min; detector, UV 254 nm. This resulted in 3-[(3-iodophenyl)carbamoyl]phenyl sulfurofluoridate (150 mg, 26.00% yield) as a white solid.

Step 2: To a stirred mixture of AgSbF₆ (118.30 mg, 0.344 mmol, 1 equiv) in DCM (6 mL) were added 3-[(3-iodophenyl)carbamoyl]phenyl sulfurofluoridate (145 mg, 0.344 mmol, 1 equiv) and 2-[(chloroaurio)dicyclohexyl-lambda5-phosphanyl]-N,N-dimethylaniline (189.30 mg, 0.344 mmol, 1 equiv) in portions at 0° C. The resulting mixture was stirred at 25° C. for additional 2 h. The resulting mixture was filtered, and the filter cake was washed with DCM. The filtrate was concentrated under reduced pressure, and added DCM (1 mL) to the concentrated filtrate. The system was purified by trituration with MTBE (4 mL). This resulted in product (55 mg, 18.93% yield, 99.54% purity) as a light grey solid. LCMS:(ES, m/z): [M]⁺=843.1.

¹H NMR (300 MHz, DMSO-d₆, ppm) δ 10.61 (s, 1H), 8.36 (dd, J=8.4, 4.4 Hz, 1H), 8.22-7.99 (m, 5H), 7.87-7.75 (m, 3H), 7.44-7.34 (m, 2H), 7.31-7.23 (m, 1H), 3.45 (s, 6H), 3.62-3.02 (m, 2H), 1.90-1.46 (m, 12H), 1.30 (d, J=31.8 Hz, 4H), 1.05 (s, 2H), 0.71-0.48 (s, 2H).

### Example 19. Synthesis of Au-MP-4

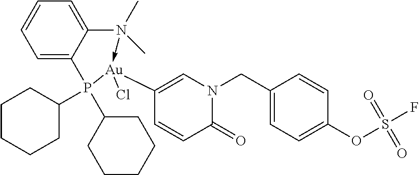

Step 1: To a stirred solution of 2(1H)-pyridinone, 5-iodo- (500 mg, 2.262 mmol, 1 equiv) and K₂CO₃ (469.03 mg, 3.393 mmol, 1.5 equiv) in DMF (5 mL) were added 4-methoxybenzyl chloride (425.19 mg, 2.714 mmol, 1.2 equiv) dropwise at 0° C. under nitrogen atmosphere. The resulting mixture was stirred at room temperature for additional 3 h. The resulting mixture was diluted with 5 mL H₂O and extracted with EtOAc (5 mL×3). The combined organic layers were washed with brine (5 mL), dried over anhydrous Na₂SO₄. After filtration, the filtrate was concentrated under reduced pressure. The residue was purified by silica gel column chromatography, eluted with PE/EA (3:1) to afford 5-iodo-1-[(4-methoxyphenyl) methyl]pyridin-2-one (500 mg, 64.78% yield) as a light yellow oil.

Step 2: To a stirred solution of 5-iodo-1-[(4-methoxyphenyl) methyl]pyridin-2-one (500 mg, 1.466 mmol, 1 equiv) in DCM (3 mL) were added Boron tribromide 1M solution in methylene chloride (6 mL) dropwise at 0° C. under nitrogen atmosphere. The resulting mixture was stirred at 0° C. for additional 1 h. The reaction was quenched with ice water at 0° C. The resulting mixture was stirred at room temperature for additional 3 h. The resulting mixture was diluted with 10 mL H₂O and extracted with DCM (10 mL×3). The combined organic layers were washed with brine (10 mL), dried over anhydrous Na₂SO₄. After filtration, the filtrate was concentrated under reduced pressure. This resulted in 1-[(4-hydroxyphenyl) methyl]-5-iodopyridin-2-one (300 mg, 62.57% yield) as a light brown solid. The crude product was used in the next step directly without further purification.

Step 3: To a stirred solution of 1-[(4-hydroxyphenyl)methyl]-5-iodopyridin-2-one (300 mg, 0.917 mmol, 1 equiv) and 3-(fluorosulfonyl)-1,2-dimethylimidazol-1-ium triflate (451.55 mg, 1.376 mmol, 1.5 equiv) in MeCN (5 mL) were added TEA (185.61 mg, 1.834 mmol, 2 equiv) dropwise at 0° C. under nitrogen atmosphere. The resulting mixture was stirred at room temperature for additional 30 min. The resulting mixture was concentrated under reduced pressure. The residue was purified by reversed-phase flash chromatography with the following conditions: column, C18 silica gel; mobile phase, MeCN in Water (0.1% FA), 10% to 90% gradient in 10 min; detector, UV 254 nm. This resulted in 4-[(5-iodo-2-oxopyridin-1-yl) methyl]phenyl sulfurofluoridate (150 mg, 39.97% yield) as a light yellow solid.

Step 4: To a stirred solution of Silver Hexafluoroantimonate(V) (117.57 mg, 0.342 mmol, 1 equiv) in DCM (3 mL) at 0° C. under nitrogen atmosphere. To the above mixture was added 4-[(5-iodo-2-oxopyridin-1-yl)methyl]phenyl sulfurofluoridate (140 mg, 0.342 mmol, 1 equiv) and 2-[(chloroaurio)dicyclohexyl-lambda5-phosphanyl]-N,N-dimethylaniline (188.14 mg, 0.342 mmol, 1 equiv) in portions at 0° C. The resulting mixture was stirred at room temperature for additional overnight. The resulting mixture was filtered, the filter cake was washed with DCM (1 mL×3). The filtrate was concentrated under reduced pressure, and added DCM (1 mL) to the concentrated filtrate. The system was purified by trituration with MTBE (4 mL). This resulted in 2-chloro-3,3-dicyclohexyl-2-[1-({4-[(fluorosulfonyl)oxy]phenyl}methyl)-6-oxopyridin-3-yl]-1,1-dimethyl-2H,3H-3lambda5-benzo[d]1-aza-3lambda5-phospha-2-auracyclopentan-1-ium-3-ylium-2,2-diuide (40 mg, 14.05% yield, 95% purity) as a light brown solid. LCMS:(ES, m/z): [M]⁺=831.1.

¹H NMR (300 MHz, DMSO-d₆, ppm) δ 8.43-8.30 (m, 1H), 8.13-7.99 (m, 2H), 7.86-7.72 (m, 1H), 7.66 (td, J=8.3, 2.4 Hz, 3H), 7.61-7.38 (m, 3H), 6.69 (dd, J=31.1, 9.6 Hz, 1H), 5.56-4.98 (m, 2H), 3.58-3.11 (m, 6H), 2.91 (s, 2H), 2.03 (s, 1H), 1.88-1.33 (m, 12H), 1.26-1.05 (d, J=38.8 Hz, 4H), 0.94-0.64 (d, J=46.3 Hz, 3H).

### Example 20. Synthesis of Au-MP-5

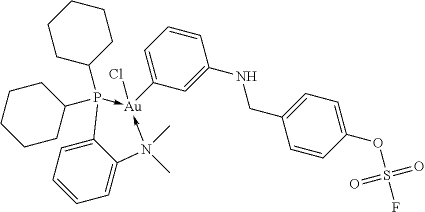

Step 1: To a stirred solution of P-hydroxybenzaldehyde (1 g, 8.188 mmol, 1 equiv) and 3-(fluorosulfonyl)-1,2-dimethylimidazol-1-ium triflate (4.03 g, 12.282 mmol, 1.5 equiv) in MeCN (10 mL) was added TEA (1.66 g, 16.376 mmol, 2 equiv) dropwise at 0° C. under nitrogen atmosphere. The resulting mixture was stirred at room temperature for additional 30 min. The resulting mixture was concentrated under reduced pressure. The residue was purified by reversed-phase flash chromatography with the following conditions: column, C18 silica gel; mobile phase, MeCN in Water (0.1% FA), 10% to 90% gradient in 10 min; detector, UV 254 nm. This resulted in 4-formylphenyl sulfurofluoridate (600 mg, 35.89% yield) as a light yellow oil.

Step 2: To a stirred solution of 4-formylphenyl sulfurofluoridate (200 mg, 0.980 mmol, 1 equiv), benzenamine, 3-iodo- (257.46 mg, 1.176 mmol, 1.2 equiv) and HOAc (58.83 mg, 0.980 mmol, 1 equiv) in DCM (4 mL) at room temperature under nitrogen atmosphere. The mixture was stirred for 15 min. STAB (415.22 mg, 1.960 mmol, 2 equiv) was added and stirred for 2 h. The resulting mixture was diluted with 5 mL H₂O and extracted with EtOAc (5 mL×3). The combined organic layers were washed with brine (5 mL), dried over anhydrous Na₂SO₄. After filtration, the filtrate was concentrated under reduced pressure. The residue was purified by reversed-phase flash chromatography with the following conditions: column, C18 silica gel; mobile phase, MeCN in Water (0.1% FA), 10% to 90% gradient in 10 min; detector, UV 254 nm. This resulted in 4-{[(3-iodophenyl) amino]methyl}phenyl sulfurofluoridate (100 mg, 25.07% yield) as a light yellow oil.

Step 3: To a stirred solution of Silver Hexafluoroantimonate(V) (84.39 mg, 0.246 mmol, 1 equiv) in DCM (3 mL) at 0° C. under nitrogen atmosphere. To the above mixture was added 4-{[(3-iodophenyl) amino]methyl}phenyl sulfurofluoridate (100 mg, 0.246 mmol, 1 equiv) and 2-[(chloroaurio)dicyclohexyl-lambda5-phosphanyl]-N,N-dimethylaniline (135.04 mg, 0.246 mmol, 1 equiv) in portions at 0° C. The resulting mixture was stirred at room temperature for additional overnight. The resulting mixture was filtered, the filter cake was washed with DCM (1 mL×3). The filtrate was concentrated under reduced pressure, and added DCM (1 mL) to the concentrated filtrate. The system was purified by trituration with MTBE (4 mL). This resulted in [(2-{[chloro({3-[({4-[(fluorosulfonyl) oxy]phenyl}methyl) amino]phenyl}) aurio]dicyclohexyl-lambda5-phosphanyl}phenyl) dimethylammonio]methanidylidene (64 mg, 30.94% yield, 92.7% purity) as a light brown solid. LCMS:(ES, m/z): [M]⁺=829.1.

¹H NMR (300 MHz, DMSO-d₆, ppm) δ 8.31 (dd, J=8.6, 4.4 Hz, 1H), 8.09-7.94 (m, 2H), 7.75 (td, J=7.5, 2.3 Hz, 1H), 7.54 (s, 4H), 7.02 (dd, J=15.0, 7.1 Hz, 1H), 6.90-6.60 (m, 2H), 6.59-6.29 (m, 2H), 4.38 (s, 2H), 3.30 (d, J=43.2 Hz, 6H), 2.88 (d, J=11.6 Hz, 2H), 1.86-1.36 (m, 12H), 1.32-1.12 (m, 4H), 1.01 (t, J=12.3 Hz, 2H), 0.61 (s, 2H).

### Example 21. Synthesis of Au-MP-6

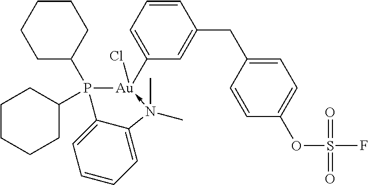

Step 1: To a stirred solution of 3-iodobenzoyl chloride (2 g, 7.506 mmol, 1 equiv) and AlCl₃ (2.20 g, 16.513 mmol, 2.2 equiv) in DCM (20 mL) was added anisole (0.89 g, 8.257 mmol, 1.1 equiv) dropwise at 0° C. under nitrogen atmosphere. The resulting mixture was stirred at room temperature for additional 2 h. The reaction was quenched by the addition of ice water (20 mL) at 0° C. The resulting mixture was extracted with DCM (20 mL×3). The combined organic layers were washed with brine (20 mL), dried over anhydrous Na₂SO₄. After filtration, the filtrate was concentrated under reduced pressure. The residue was purified by silica gel column chromatography, eluted with PE/EA (4:1) to afford (3-iodophenyl) (4-methoxyphenyl) methanone (1.5 g, 59.10% yield) as alight yellow oil.

Step 2: To a stirred solution of (3-iodophenyl)(4-methoxyphenyl)methanone (1.5 g, 4.436 mmol, 1 equiv) in MeCN (20 mL) at 0° C. under nitrogen atmosphere. To the above mixture was added Et₃SiH (1.16 g, 9.981 mmol, 2.25 equiv) and Boron trifluoride in diethyl ether solution (3.35 g, 11.090 mmol, 2.5 equiv, 47%) dropwise at 0° C. The resulting mixture was stirred at room temperature for additional overnight. The mixture was basified to pH 7 with saturated NaHCO₃ (aq.). The resulting mixture was diluted with 20 mL H₂O and extracted with EtOAc (20 mL×3). The combined organic layers were washed with brine (20 mL), dried over anhydrous Na₂SO₄. After filtration, the filtrate was concentrated under reduced pressure. The residue was purified by silica gel column chromatography, eluted with PE/EA (5:1) to afford 1-iodo-3-[(4-methoxyphenyl) methyl]benzene (1.1 g, 76.50% yield) as a light yellow solid.

Step 3: To a stirred solution of 1-iodo-3-[(4-methoxyphenyl) methyl]benzene (1.1 g, 3.393 mmol, 1 equiv) in DCM (3 mL) was added Boron tribromide 1M solution in methylene chloride (9 mL) dropwise at 0° C. under nitrogen atmosphere. The resulting mixture was stirred at 0° C. for additional 1 h. The reaction was quenched by the addition of ice water (10 mL) at 0° C. The resulting mixture was extracted with DCM (20 mL×3). The combined organic layers were washed with brine (20 mL), dried over anhydrous Na₂SO₄. After filtration, the filtrate was concentrated under reduced pressure. This resulted in 4-[(3-iodophenyl) methyl]phenol (700 mg, 66.51% yield) as a light brown solid. The crude product was used in the next step directly without further purification.

Step 4: To a stirred solution of 4-[(3-iodophenyl) methyl]phenol (700 mg, 2.257 mmol, 1 equiv) and 3-(fluorosulfonyl)-1,2-dimethylimidazol-1-ium triflate (1111.33 mg, 3.386 mmol, 1.5 equiv) in MeCN (10 mL) was added TEA (456.80 mg, 4.514 mmol, 2 equiv) dropwise at 0° C. under nitrogen atmosphere. The resulting mixture was stirred at room temperature for additional 30 min. The residue was purified by reversed-phase flash chromatography with the following conditions: column, C18 silica gel; mobile phase, MeCN in Water (0.1% FA), 10% to 90% gradient in 10 min; detector, UV 254 nm. This resulted in 4-[(3-iodophenyl) methyl]phenyl sulfurofluoridate (400 mg, 45.19% yield) as a light yellow oil.

Step 5: To a stirred solution of Silver Hexafluoroantimonate(V) (87.62 mg, 0.255 mmol, 1 equiv) in DCM (3 mL) at 0° C. under nitrogen atmosphere. To the above mixture was added 4-[(3-iodophenyl) methyl]phenyl sulfurofluoridate (100 mg, 0.255 mmol, 1 equiv) and 2-[(chloroaurio)dicyclohexyl-lambda5-phosphanyl]-N,N-dimethylaniline (140.21 mg, 0.255 mmol, 1 equiv) in portions at 0° C. The resulting mixture was stirred at room temperature for additional overnight. The resulting mixture was filtered, the filter cake was washed with DCM (1 mL×3). The filtrate was concentrated under reduced pressure, and added DCM (2 mL) to the concentrated filtrate. The system was purified by trituration with MTBE (4 mL). This resulted in 2-chloro-3,3-dicyclohexyl-2-[3-({4-[(fluorosulfonyl) oxy]phenyl}methyl) phenyl]-1,1-dimethyl-2H,3H-3lambda5-benzo[d]1-aza-3lambda5-phospha-2-auracyclopentan-1-ium-3-ylium-2,2-diuide (100 mg, 48.11% yield, 93.9% purity) as a grey solid. LCMS:(ES, m/z): [M]⁺=814.1.

¹H NMR (300 MHz, DMSO-d₆, ppm) δ 8.34 (dd, J=8.6, 4.4 Hz, 1H), 8.03 (q, J=7.7 Hz, 2H), 7.76 (td, J=7.4, 2.4 Hz, 1H), 7.57 (d, J=8.5 Hz, 2H), 7.51-7.42 (m, 2H), 7.40-7.12 (m, 4H), 4.08 (s, 2H), 3.08-2.55 (m, 2H), 1.59 (d, J=34.0 Hz, 12H), 1.21 (d, J=19.7 Hz, 4H), 0.91 (s, 2H), 0.47 (s, 2H).

### Example 22. Synthesis of Au-MP-8

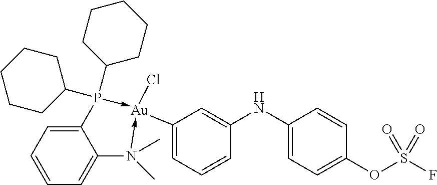

Step 1: Into a 100 mL 2-necked round-bottom flask were added 1,3-diiodobenzene (1.5 g, 4.547 mmol, 1 equiv), P-anisidine (0.56 g, 4.547 mmol, 1 equiv), Pd(dppf)Cl₂ (0.33 g, 0.455 mmol, 0.1 equiv), t-BuONa (1.31 g, 13.641 mmol, 3 equiv) in toluene (50 mL) at room temperature. The resulting mixture was stirred at 90° C. for additional 36 h. The reaction was monitored by LCMS. The resulting mixture was extracted with EtOAc (3×20 mL). The combined organic layers were washed with water (3×20 mL), dried over anhydrous Na₂SO₄. After filtration, the filtrate was concentrated under reduced pressure. The residue was purified by reversed-phase flash chromatography with the following conditions: column, C18 silica gel; mobile phase, MeCN in Water (0.1% FA), 10% to 60% gradient in 12 min; detector, UV 254 nm. This resulted in 3-iodo-N-(4-methoxyphenyl)aniline (640 mg, 43.29% yield) as a black oil.

Step 2: To a stirred mixture of 3-iodo-N-(4-methoxyphenyl)aniline (650 mg, 1.999 mmol, 1 equiv) in DCM was added BBr₃ (10 mL) dropwise at 0° C. The resulting mixture was stirred at room temperature for additional 20 min. The reaction was quenched with ice water at 0° C. The resulting mixture was extracted with DCM (3×10 mL). The combined organic layers were washed with water (1×5 mL), dried over anhydrous Na₂SO₄. After filtration, the filtrate was concentrated under reduced pressure. The crude product mixture was used in the next step directly without further purification.

Step 3: To a stirred mixture of 4-[(3-iodophenyl)amino]phenol (460 mg, 1.479 mmol, 1 equiv) and 3-(fluorosulfonyl)-1,2-dimethylimidazol-1-ium triflate (582.39 mg, 1.775 mmol, 1.2 equiv) in ACN (10 mL) were added TEA (299.23 mg, 2.958 mmol, 2 equiv) dropwise at 0° C. The resulting mixture was stirred at room temperature for additional 30 min. The reaction was monitored by LCMS. The residue was purified by reversed-phase flash chromatography with the following conditions: column, C18 silica gel; mobile phase, MeCN in Water (0.1% FA), 10% to 70% gradient in 12 min; detector, UV 254 nm. This resulted in 4-[(3-iodophenyl)amino]phenyl sulfurofluoridate (280 mg, 48.17% yield) as a colorless oil.

Step 4: To a stirred mixture of AgSbF₆ (61.18 mg, 0.178 mmol, 1.00 equiv) in DCM (4 mL) were added 4-[(3-iodophenyl) amino]phenyl sulfurofluoridate (70 mg, 0.178 mmol, 1.00 equiv) and (chloroaurio)dicyclohexyl[2-(dimethylamino) phenyl]-lambda5-phosphanyl (97.90 mg, 0.178 mmol, 1.00 equiv) dropwise at 0° C. The resulting mixture was stirred at 25° C. for additional 2 h. The resulting mixture was filtered, and the filter cake was washed with DCM. The filtrate was concentrated under reduced pressure, and added DCM (1 mL) to the concentrated filtrate. The system was purified by trituration with MTBE (4 mL). This resulted in {chloro[3-({4-[(fluorosulfonyl) oxy]phenyl}amino) phenyl]aurio}dicyclohexyl[2-(dimethylamino) phenyl]-lambda5-phosphanyl (50 mg, 34.41% yield, 99.57% purity) as a light yellow solid. LCMS: (ES, m/z): [M]⁺=815.0.

¹H NMR (300 MHz, DMSO-d₆, ppm) δ 8.66 (s, 1H), 8.34 (dd, J=8.5, 4.4 Hz, 1H), 8.12-7.97 (m, 2H), 7.83-7.73 (m, 1H), 7.47 (d, J=8.9 Hz, 2H), 7.28 (t, J=7.9 Hz, 1H), 7.24-7.14 (m, 3H), 7.09-7.00 (m, 1H), 6.93 (d, J=7.8 Hz, 1H), 3.42 (s, 6H), 2.94 (d, J=11.4 Hz, 2H), 1.96-1.41 (m, 12H), 1.25 (s, 4H), 1.01 (s, 2H), 0.65 (s, 2H).

### Example 23. Synthesis of Au-PM-3A and Au-PM-3B

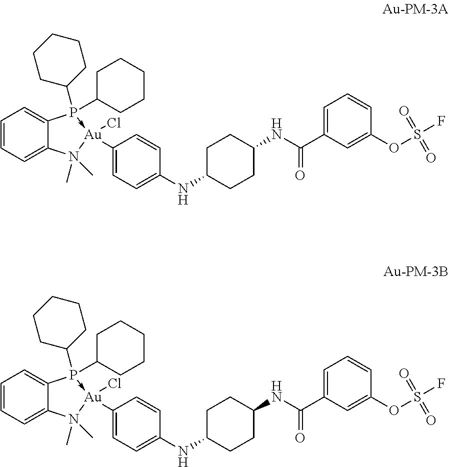

Step 1: To a stirred solution of tert-butyl N-(4-oxocyclohexyl)carbamate (2 g, 9.377 mmol, 1 equiv), benzenamine, 4-iodo- (2.46 g, 11.252 mmol, 1.2 equiv) and HOAc (0.56 g, 9.377 mmol, 1 equiv) in DCM (20 mL) at room temperature under nitrogen atmosphere. To the above mixture was added STAB (3.97 g, 18.754 mmol, 2 equiv) in portions at room temperature. The resulting mixture was stirred at room temperature for additional 2 h. The resulting mixture was diluted with 20 mL H₂O and extracted with DCM (20 mL×3). The combined organic layers were washed with brine (20 mL), dried over anhydrous Na₂SO₄. After filtration, the filtrate was concentrated under reduced pressure. This resulted in tert-butyl N-{4-[(4-iodophenyl) amino]cyclohexyl}carbamate (3.5 g, 89.65% yield) as a light yellow solid. The crude product was used in the next step directly without further purification.

Step 2: To a stirred solution of tert-butyl N-{4-[(4-iodophenyl) amino]cyclohexyl}carbamate (4 g, 9.608 mmol, 1 equiv) in DCM (20 mL) were added tert-butyl N-{4-[(4-iodophenyl)amino]cyclohexyl}carbamate (4 g, 9.608 mmol, 1 equiv) dropwise at room temperature under nitrogen atmosphere. The resulting mixture was stirred at room temperature for additional 2 h. The precipitated solids were collected by filtration and washed with DCM (5 mL×3). This resulted in N1-(4-iodophenyl) cyclohexane-1,4-diamine hydrochloride (3 g, 88.54% yield) as a light yellow solid. The crude product was used in the next step directly without further purification.

Step 3: To a stirred solution 3-[(fluorosulfonyl)oxy]benzoic acid (522.25 mg, 2.372 mmol, 1.5 equiv), HATU (901.93 mg, 2.372 mmol, 1.5 equiv) and DIEA (408.77 mg, 3.162 mmol, 2 equiv) in DCM (5 mL) at room temperature under nitrogen atmosphere. The mixture was stirred for 15 min. N1-(4-iodophenyl) cyclohexane-1,4-diamine (500 mg, 1.581 mmol, 1 equiv) was added and stirred for 30 min. The resulting mixture was diluted with 5 mL H₂O and extracted with DCM (5 mL×3). The combined organic layers were washed with brine (5 mL), dried over anhydrous Na₂SO₄. After filtration, the filtrate was concentrated under reduced pressure. The residue was purified by reversed-phase flash chromatography with the following conditions: column, C18 silica gel; mobile phase, MeCN in Water (0.1% FA), 10% to 50% gradient in 10 min; detector, UV 254 nm. This resulted in 3-{[(1s,4s)-4-[(4-iodophenyl) amino]cyclohexyl]carbamoyl}phenyl sulfurofluoridate (100 mg, 12.20% yield) and 3-{[(1r,4r)-4-[(4-iodophenyl)amino]cyclohexyl]carbamoyl}phenyl sulfurofluoridate (140 mg, 17.08% yield) as a light yellow oil.

Step 4A: To a stirred solution of Silver Hexafluoroantimonate(V) (66.29 mg, 0.193 mmol, 1 equiv) in DCM (3 mL) at 0° C. under nitrogen atmosphere. To the above mixture was added 3-{[(1s,4s)-4-[(4-iodophenyl)amino]cyclohexyl]carbamoyl}phenyl sulfurofluoridate (100 mg, 0.193 mmol, 1 equiv) and (chloroaurio)dicyclohexyl[2-(dimethylamino)phenyl]-lambda5-phosphanyl (106.08 mg, 0.193 mmol, 1 equiv) in portions at 0° C. The resulting mixture was stirred at room temperature for additional overnight. The resulting mixture was stirred at room temperature for additional overnight. The resulting mixture was filtered, the filter cake was washed with DCM (1 mL×3). The filtrate was concentrated under reduced pressure, and added DCM (1 mL) to the concentrated filtrate. The system was purified by trituration with MTBE (4 mL). This resulted in 2-chloro-3,3-dicyclohexyl-1,1-dimethyl-2-(4-{[(1s,4s)-4-{3-[(fluorosulfonyl)oxy]benzamido}cyclohexyl]amino}phenyl)-2H,3H-3lambda5-benzo[d]1-aza-3lambda5-phospha-2-auracyclopentan-1-ium-3-ylium-2,2-diuide (80 mg, 44.05% yield, 97.2% purity) as a light yellow solid. LCMS Au-PM-3-0A:(ES, m/z): [M]⁺=940.1 ¹H NMR Au-PM-3A (300 MHz, DMSO-d₆, ppm) δ 8.52 (d, J=7.7 Hz, 1H), 8.33 (dd, J=8.5, 4.4 Hz, 1H), 8.12-7.96 (m, 4H), 7.88-7.47 (m, 4H), 7.04 (s, 2H), 6.76 (d, J=29.5 Hz, 2H), 3.74 (d, J=27.1 Hz, 6H), 3.26 (s, 2H), 2.92 (d, J=11.6 Hz, 2H), 2.05 (d, J=12.1 Hz, 2H), 1.94 (d, J=11.9 Hz, 2H), 1.83 (s, 2H), 1.78-1.57 (m, 8H), 1.47 (t, J=12.0 Hz, 4H), 1.29 (dd, J=22.9, 11.1 Hz, 6H), 1.05 (t, J=12.1 Hz, 2H), 0.64 (s, 2H).

Step 4B: To a stirred solution of Silver Hexafluoroantimonate(V) (92.81 mg, 0.270 mmol, 1 equiv) in DCM (3 mL) at 0° C. under nitrogen atmosphere. To the above mixture was added 3-{[(1r,4r)-4-[(4-iodophenyl)amino]cyclohexyl]carbamoyl}phenyl sulfurofluoridate (140 mg, 0.270 mmol, 1 equiv) and (chloroaurio)dicyclohexyl[2-(dimethylamino)phenyl]-lambda5-phosphanyl (148.52 mg, 0.270 mmol, 1 equiv) in portions at 0° C. The resulting mixture was filtered, the filter cake was washed with DCM (1 mL×3). The filtrate was concentrated under reduced pressure, and added DCM (1 mL) to the concentrated filtrate. The system was purified by trituration with MTBE (4 mL). This resulted in 2-chloro-3,3-dicyclohexyl-1,1-dimethyl-2-(4-{[(1r,4r)-4-{3-[(fluorosulfonyl) oxy]benzamido}cyclohexyl]amino}phenyl)-2H,3H-3lambda5-benzo[d]1-aza-3lambda5-phospha-2-auracyclopentan-1-ium-3-ylium-2,2-diuide (80 mg, 31.47% yield, 97.4% purity) as a light yellow solid. LCMS Au-PM-3-0B:(ES, m/z): [M]⁺=940.2 ¹H NMR Au-PM-3B (300 MHz, DMSO-d₆, ppm) δ 8.42 (d, J=6.6 Hz, 1H), 8.33 (dd, J=8.6, 4.3 Hz, 1H), 8.16-7.97 (m, 4H), 7.87-7.58 (m, 4H), 7.06 (s, 2H), 6.87-6.64 (m, 2H), 4.13 (s, 6H), 3.91 (s, 2H), 3.46 (s, 1H), 2.90 (d, J=11.0 Hz, 1H), 2.00-1.41 (m, 20H), 1.32 (dd, J=24.0, 11.9 Hz, 4H), 1.05 (t, J=12.2 Hz, 2H), 0.63 (s, 2H).

### Example 24. Synthesis of Au-PM-5

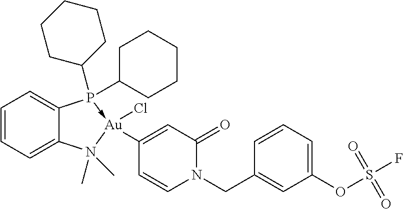

Step 1: A mixture of 4-iodo-1H-pyridin-2-one (1 g, 4.525 mmol, 1 equiv) and 1-(bromomethyl)-3-methoxybenzene (1.09 g, 5.430 mmol, 1.2 equiv) in DMF (20 mL) was stirred at room temperature for 1 h. The resulting mixture was filtered, the filter cake was washed with MeOH (3×3 mL). The filtrate was concentrated under reduced pressure. The residue was purified by reversed-phase flash chromatography with the following conditions: column, C18 silica gel; mobile phase, MeCN in Water (0.1% FA), 10% to 95% gradient in 10 min; detector, UV 254 nm. This resulted in 4-iodo-1-[(3-methoxyphenyl) methyl]pyridin-2-one (800 mg, 51.82% yield) as a white solid.

Step 2: To a stirred mixture of 4-iodo-1-[(3-methoxyphenyl) methyl]pyridin-2-one (500 mg, 1.466 mmol, 1 equiv) in DCM (2 mL) was added BBr3 (4 mL) dropwise at 0° C. The resulting mixture was stirred at 0° C. for 1 h. The reaction mixture was quenched by ice water and extracted with DCM (3×20 mL), dried over anhydrous Na₂SO₄. After filtration, the filtrate was concentrated under reduced pressure to afford 1-[(3-hydroxyphenyl) methyl]-4-iodopyridin-2-one (400 mg, 83.43% yield) as a white solid. The crude resulting mixture was used in the next step directly without further purification.

Step 3: To a stirred mixture of 1-[(3-hydroxyphenyl) methyl]-4-iodopyridin-2-one (300 mg, 0.917 mmol, 1 equiv) and 3-(fluorosulfonyl)-1,2-dimethylimidazol-1-ium triflate (361.24 mg, 1.100 mmol, 1.2 equiv) in MeCN (5 mL) was added TEA (139.20 mg, 1.376 mmol, 1.5 equiv) dropwise at 0° C. The resulting mixture was stirred at 0° C. for 30 min. The resulting mixture was concentrated under reduced pressure. The residue was purified by reversed-phase flash chromatography with the following conditions: column, C18 silica gel; mobile phase, MeCN in Water (0.1% FA), 10% to 95% gradient in 10 min; detector, UV 254 nm. This resulted in 3-[(4-iodo-2-oxopyridin-1-yl) methyl]phenyl sulfurofluoridate (200 mg, 53.30% yield) as a white solid.

Step 4: To a stirred solution of 3-[(4-iodo-2-oxopyridin-1-yl) methyl]phenyl sulfurofluoridate (100 mg, 0.244 mmol, 1 equiv) and (chloroaurio)dicyclohexyl[2-(dimethylamino) phenyl]-lambda5-phosphanyl (134.39 mg, 0.244 mmol, 1 equiv) in DCM (5 mL) were added Silver Hexafluoroantimonate(V) (83.98 mg, 0.244 mmol, 1 equiv) in portions at 0° C. under nitrogen atmosphere. The resulting mixture was stirred at room temperature for additional 2 h. The reaction was monitored by LCMS. The resulting mixture was filtered, the filter cake was washed with DCM (3×3 mL). The filtrate was concentrated under reduced pressure, and added DCM (1 mL) to the concentrated filtrate. The system was purified by trituration with MTBE (4 mL). This resulted in {chloro[1-({3-[(fluorosulfonyl) oxy]phenyl}methyl)-2-oxopyridin-4-yl]aurio}dicyclohexyl[2-(dimethylamino) phenyl]-lambda5-phosphanyl (50 mg, 24.59% yield, 93.3% purity) as a grey solid. LCMS:(ES, m/z): [M]⁺=831.1.

¹H NMR (300 MHz, DMSO-d₆, ppm) δ 8.36 (dd, J=8.6, 4.4 Hz, 1H), 8.03 (d, J=8.6 Hz, 2H), 7.79 (t, J=7.2 Hz, 1H), 7.69-7.53 (m, 4H), 7.50 (s, 1H), 7.44 (d, J=7.3 Hz, 1H), 6.64 (d, J=9.6 Hz, 1H), 5.28 (s, 2H), 2.67 (s, 2H), 1.83 (s, 2H), 1.70-1.48 (m, 10H), 1.27-1.14 (m, 4H), 0.98 (s, 2H), 0.83 (s, 2H).

### Example 25. Synthesis of Au-PM-7

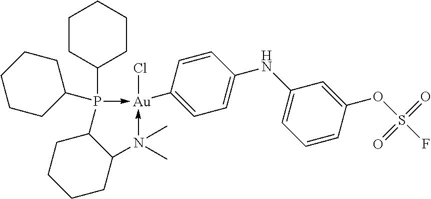

Step 1: Into a 100 mL 2-necked round-bottom flask were added 1,4-diiodobenzene (1.5 g, 4.547 mmol, 1 equiv), M-anisidine (0.56 g, 4.547 mmol, 1 equiv), Pd(dppf)Cl₂ (332.69 mg, 0.455 mmol, 0.1 equiv), t-OBuNa (1.31 g, 13.641 mmol, 3 equiv) in toluene (50 mL) at room temperature. The resulting mixture was stirred at 90° C. for additional 36 h. The reaction was monitored by LCMS. The resulting mixture was extracted with EtOAc (3×20 mL). The combined organic layers were washed with water (3×20 mL), dried over anhydrous Na₂SO₄. After filtration, the filtrate was concentrated under reduced pressure. The residue was purified by reversed-phase flash chromatography with the following conditions: column, C18 silica gel; mobile phase, MeCN in Water (0.1% FA), 10% to 60% gradient in 12 min; detector, UV 254 nm. This resulted in 4-iodo-N-(3-methoxyphenyl)aniline (500 mg, 33.82% yield) as a black oil.

Step 2: To a stirred mixture of 4-iodo-N-(3-methoxyphenyl)aniline (500 mg, 1.538 mmol, 1 equiv) in DCM was added BBr₃ (8 mL) dropwise at 0° C. The resulting mixture was stirred at room temperature for additional 20 min. The reaction was quenched with ice water at 0° C. The resulting mixture was extracted with DCM (3×10 mL). The combined organic layers were washed with water (1×5 mL), dried over anhydrous Na₂SO₄. After filtration, the filtrate was concentrated under reduced pressure. The crude product mixture was used in the next step directly without further purification.

Step 3: To a stirred mixture of 3-[(4-iodophenyl)amino]phenol (370 mg, 1.189 mmol, 1 equiv) and 3-(fluorosulfonyl)-1,2-dimethylimidazol-1-ium triflate (468.44 mg, 1.427 mmol, 1.2 equiv) in ACN (8 mL) were added TEA (240.69 mg, 2.379 mmol, 2 equiv) dropwise at 0° C. The resulting mixture was stirred at room temperature for additional 30 min. The reaction was monitored by LCMS. The residue was purified by reversed-phase flash chromatography with the following conditions: column, C18 silica gel; mobile phase, MeCN in Water (0.1% FA), 10% to 70% gradient in 12 min; detector, UV 254 nm. This resulted in 3-[(4-iodophenyl) amino]phenyl sulfurofluoridate (160 mg, 34.22% yield) as a colorless oil.

Step 4: To a stirred mixture of AgSbF₆ (61.18 mg, 0.178 mmol, 1 equiv) in DCM (3 mL) were added 3-[(4-iodophenyl)amino]phenyl sulfurofluoridate (70 mg, 0.178 mmol, 1 equiv) and (chloroaurio)dicyclohexyl[2-(dimethylamino)phenyl]-lambda5-phosphanyl (97.90 mg, 0.178 mmol, 1 equiv) dropwise at 0° C. The resulting mixture was stirred at 25° C. for additional 2 h. The resulting mixture was filtered, and the filter cake was washed with DCM, and added DCM (0.5 mL) to the concentrated filtrate. The system was purified by trituration with MTBE (2 mL). The residue was purified by trituration with MTBE (2 mL). This resulted in product (20 mg, 13.76% yield, 94.28% purity) as a yellow solid. LCMS: (ES, m/z): [M]⁺=815.1.

¹H NMR (400 MHz, DMSO-d₆, ppm) δ 8.77 (s, 1H), 8.35 (dd, J=8.6, 4.3 Hz, 1H), 8.05 (dt, J=15.7, 8.3 Hz, 2H), 7.79 (t, J=7.5 Hz, 1H), 7.44 (t, J=8.3 Hz, 1H), 7.32 (d, J=8.4 Hz, 2H), 7.20 (d, J=8.4 Hz, 2H), 7.17-7.11 (m, 1H), 7.07 (t, J=2.3 Hz, 1H), 7.01-6.94 (m, 1H), 3.44 (s, 6H), 2.96 (d, J=11.1 Hz, 2H), 1.88 (s, 2H), 1.81-1.48 (m, 10H), 1.47-1.28 (m, 4H), 1.16-1.00 (m, 2H), 0.74-0.52 (m, 2H).

### Example 26. Synthesis of Au—PP-3A and Au—PP-3B

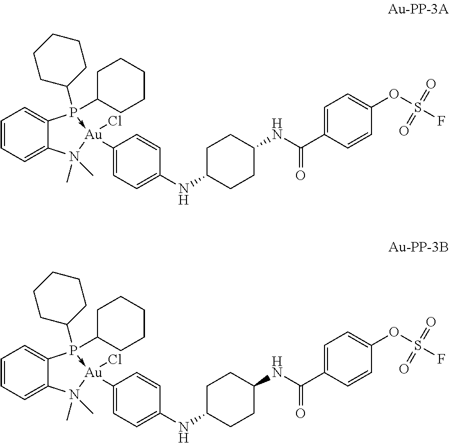

Step 1: To a stirred solution of 4-[(fluorosulfonyl)oxy]benzoic acid (468.26 mg, 2.127 mmol, 1.5 equiv), HATU (808.69 mg, 2.127 mmol, 1.5 equiv) and DIEA (366.51 mg, 2.836 mmol, 2 equiv) in DCM (5 mL) at room temperature under nitrogen atmosphere. The mixture was stirred for 15 min. N1-(4-iodophenyl) cyclohexane-1,4-diamine hydrochloride (500 mg, 1.418 mmol, 1 equiv) was added stirred for 30 min. The resulting mixture was diluted with 5 mL H₂O and extracted with DCM (5 mL×3). The combined organic layers were washed with brine (5 mL), dried over anhydrous Na₂SO₄. After filtration, the filtrate was concentrated under reduced pressure. The residue was purified by reversed-phase flash chromatography with the following conditions: column, C18 silica gel; mobile phase, MeCN in Water (0.1% FA), 10% to 50% gradient in 10 min; detector, UV 254 nm. This resulted in 4-{[(1s,4s)-4-[(4-iodophenyl) amino]cyclohexyl]carbamoyl}phenyl sulfurofluoridate (110 mg, 14.97% yield) and 4-{[(1r,4r)-4-[(4-iodophenyl)amino]cyclohexyl]carbamoyl}phenyl sulfurofluoridate (120 mg, 16.33% yield) as a light yellow solid.

Step 2A: To a stirred solution Silver Hexafluoroantimonate(V) (72.92 mg, 0.212 mmol, 1 equiv) in DCM (3 mL) at 0° C. under nitrogen atmosphere. To the above mixture was added (chloroaurio)dicyclohexyl[2-(dimethylamino)phenyl]-lambda5-phosphanyl (116.69 mg, 0.212 mmol, 1 equiv) and dropwise at 0° C. The resulting mixture was stirred at room temperature for additional 2 h. The resulting mixture was filtered, the filter cake was washed with DCM (1 mL×3). The filtrate was concentrated under reduced pressure, and added DCM (1 mL) to the concentrated filtrate. The system was purified by trituration with MTBE (4 mL). This resulted in 2-chloro-3,3-dicyclohexyl-1,1-dimethyl-2-(4-{[(1s,4s)-4-{4-[(fluorosulfonyl)oxy]benzamido}cyclohexyl]amino}phenyl)-2H,3H-3lambda5-benzo[d]1-aza-3lambda5-phospha-2-auracyclopentan-1-ium-3-ylium-2,2-diuide (Au—PP-3A; 90 mg, 45.05% yield, 95.0% purity) as a light brown solid. LCMS:(ES, m/z): [M]⁺=940.2.

¹H NMR (300 MHz, DMSO-d₆, ppm) δ 8.48 (d, J=7.6 Hz, 1H), 8.33 (dd, J=8.5, 4.3 Hz, 1H), 8.13-7.96 (m, 4H), 7.83-7.32 (m, 4H), 7.05 (s, 2H), 6.73 (s, 2H), 4.08 (s, 2H), 3.40 (s, 6H), 2.92 (d, J=10.6 Hz, 2H), 2.05 (d, J=12.3 Hz, 2H), 1.93 (d, J=12.2 Hz, 2H), 1.83 (s, 2H), 1.67 (d, J=22.4 Hz, 8H), 1.48 (d, J=12.1 Hz, 4H), 1.41-1.24 (m, 6H), 1.03 (d, J=13.2 Hz, 2H), 0.64 (s, 2H).

Step 2B: To a stirred solution of Silver Hexafluoroantimonate(V) (79.55 mg, 0.232 mmol, 1 equiv) in DCM (3 mL) at 0° C. under nitrogen atmosphere. To the above mixture was added 4-{[(1r,4r)-4-[(4-iodophenyl) amino]cyclohexyl]carbamoyl}phenyl sulfurofluoridate (120 mg, 0.232 mmol, 1 equiv) and (chloroaurio)dicyclohexyl[2-(dimethylamino)phenyl]-lambda5-phosphanyl (127.30 mg, 0.232 mmol, 1 equiv) in portions at 0° C. The resulting mixture was stirred at room temperature for additional 2 h. The resulting mixture was filtered, the filter cake was washed with DCM (1 mL×3). The filtrate was concentrated under reduced pressure, and added DCM (1 mL) to the concentrated filtrate. The system was purified by trituration with MTBE (4 mL). This resulted in 2-chloro-3,3-dicyclohexyl-1,1-dimethyl-2-(4-{[(1r,4r)-4-{4-[(fluorosulfonyl)oxy]benzamido}cyclohexyl]amino}phenyl)-2H,3H-3lambda5-benzo[d]1-aza-3lambda5-phospha-2-auracyclopentan-1-ium-3-ylium-2,2-diuide (Au—PP-3B; 90 mg, 41.30% yield, 95.0% purity) as a light brown solid. LCMS:(ES, m/z): [M]⁺=940.1.

¹H NMR (300 MHz, DMSO-d₆, ppm) δ 8.39 (d, J=6.6 Hz, 1H), 8.33 (dd, J=8.6, 4.3 Hz, 1H), 8.15-7.96 (m, 4H), 7.86-7.17 (m, 4H), 7.04 (d, J=8.2 Hz, 2H), 6.71 (d, J=8.4 Hz, 2H), 4.23 (s, 1H), 3.90 (s, 1H), 3.39 (s, 6H), 2.91 (d, J=11.2 Hz, 2H), 1.92-1.48 (d, J=29.1 Hz, 20H), 1.30 (dt, J=18.3, 9.3 Hz, 4H), 1.03 (d, J=13.0 Hz, 2H), 0.63 (s, 2H).

### Example 27. Synthesis of Au—PP-9

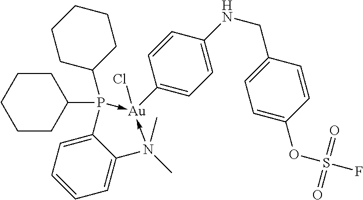

Step 1: To a stirred solution of 4-formylphenyl sulfurofluoridate (200 mg, 0.980 mmol, 1 equiv), benzenamine, 4-iodo- (257.46 mg, 1.176 mmol, 1.2 equiv) and HOAc (58.83 mg, 0.980 mmol, 1 equiv) in DCM (5 mL) at room temperature under nitrogen atmosphere. The mixture was stirred for 15 min. STAB (415.22 mg, 1.960 mmol, 2 equiv) was added and stirred for 2 h. The resulting mixture was diluted with 5 mL H₂O and extracted with EtOAc (5 mL×3). The combined organic layers were washed with brine (5 mL), dried over anhydrous Na₂SO₄. After filtration, the filtrate was concentrated under reduced pressure. The residue was purified by reversed-phase flash chromatography with the following conditions: column, C18 silica gel; mobile phase, MeCN in Water (0.1% FA), 10% to 95% gradient in 10 min; detector, UV 254 nm. This resulted in 4-{[(4-iodophenyl) amino]methyl}phenyl sulfurofluoridate (100 mg, 25.07% yield) as a light brown oil.

Step 2: To a stirred solution of Silver Hexafluoroantimonate(V) (84.39 mg, 0.246 mmol, 1 equiv) in DCM (3 mL) at 0° C. under nitrogen atmosphere. To the above mixture was added 4-{[(4-iodophenyl) amino]methyl}phenyl sulfurofluoridate (100 mg, 0.246 mmol, 1 equiv) and 2-[(chloroaurio)dicyclohexyl-lambda5-phosphanyl]-N,N-dimethylaniline (135.04 mg, 0.246 mmol, 1 equiv) in portions at 0° C. The resulting mixture was stirred at room temperature for additional overnight. The resulting mixture was filtered, the filter cake was washed with DCM (1 mL×3). The filtrate was concentrated under reduced pressure, and added DCM (1 mL) to the concentrated filtrate. The system was purified by trituration with MTBE (4 mL). This resulted in 2-chloro-3,3-dicyclohexyl-2-{4-[({4-[(fluorosulfonyl) oxy]phenyl}methyl) amino]phenyl}-1,1-dimethyl-2H,3H-3lambda5-benzo[d]1-aza-3lambda5-phospha-2-auracyclopentan-1-ium-3-ylium-2,2-diuide (60 mg, 29.43% yield, 94.4% purity) as a light brown solid. LCMS:(ES, m/z): [M]⁺=829.1.

¹H NMR (300 MHz, DMSO-d₆, ppm) δ 8.31 (dd, J=8.3, 4.4 Hz, 1H), 8.12-7.93 (m, 2H), 7.82-7.69 (m, 1H), 7.55 (d, J=3.6 Hz, 4H), 7.17 (d, J=8.4 Hz, 1H), 6.99 (d, J=8.3 Hz, 2H), 6.62 (d, J=8.5 Hz, 2H), 4.38 (s, 2H), 3.39 (d, J=8.8 Hz, 6H), 2.87 (d, J=11.2 Hz, 2H), 2.34-1.35 (m, 12H), 1.25 (td, J=25.3, 23.8, 9.2 Hz, 4H), 0.97 (t, J=13.2 Hz, 2H), 0.56 (s, 2H).

### Example 28. Synthesis of SFY—Au-2-1

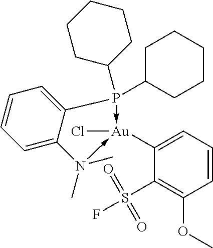

Step 1: To a stirred mixture of 2-iodo-6-methoxyaniline (1 g, 4.01 mmol, 1 equiv) and NaNO₂ (0.28 g, 4.015 mmol, 1 equiv) in EtOH (3 mL) were added Tetrafluoroboric acid (40% in H₂O) (1.94 g, 22.08 mmol, 5.5 equiv) and H₂O (1:1) dropwise at 0° C. The reaction was monitored by LCMS. The precipitated solids were collected by filtration and washed with EtOH (2 mL) (1×2 mL) to afford 2-iodo-6-methoxybenzenediazonium (800 mg, 76.33% yield) as a yellow solid.

Step 2: To a stirred solution/mixture of 2-iodo-6-methoxybenzenediazonium (800 mg, 3.06 mmol, 1 equiv) and N-(benzenesulfonyl)-S-phenylfluoranesulfonamido (966.37 mg, 3.065 mmol, 1 equiv) in ACN (4.5 mL) was added potassium metabisulfite (1362.60 mg, 6.130 mmol, 2 equiv) in portions at room temperature under nitrogen atmosphere. To the above mixture was added AcOH (0.3 mL) and H₂O (75 uL) in portions over 5 min at room temperature. The resulting mixture was stirred at room temperature for additional 6 h. The reaction was monitored by LCMS. The resulting mixture was extracted with CH₂Cl₂ (3×1 mL). The combined organic layers were washed with water (3×1 mL), dried over anhydrous Na₂SO₄. After filtration, the filtrate was concentrated under reduced pressure. The residue was purified by silica gel column chromatography, eluted with PE/EA (5:1) to afford 2-iodo-6-methoxybenzenesulfonic acid (400 mg, 41.56% yield) as a white solid.

Step 3: A solution of 2-iodo-6-methoxybenzenesulfonic acid (400 mg, 1.274 mmol, 1 equiv) in DCM (4 mL) was treated with DAST (41.06 mg, 0.255 mmol, 0.2 equiv) at room temperature for 1 h under nitrogen atmosphere. The reaction was monitored by LCMS. The reaction was quenched by the addition of ice water (4 mL) at room temperature. The combined organic layers were washed with water (2×1 mL), dried over anhydrous Na₂SO₄. After filtration, the filtrate was concentrated under reduced pressure. The residue was purified by silica gel column chromatography, eluted with PE/EA (5:1) to afford 2-iodo-6-methoxybenzenesulfonyl fluoride (170 mg, 42.23% yield) as a yellow solid.

Step 4: Into a 40 mL sealed tube were added Silver Hexafluoroantimonate(V) (184.81 mg, 0.538 mmol, 1 equiv) and DCM (5 mL) at 0° C. To the above mixture was added 2-[(chloroaurio)dicyclohexyl-lambda5-phosphanyl]-N,N-dimethylaniline (296.28 mg, 0.538 mmol, 1 equiv) and 2-iodo-6-methoxybenzenesulfonyl fluoride (170 mg, 0.538 mmol, 1 equiv) dropwise over 3 min at 0° C. The resulting mixture was stirred at room temperature for additional overnight. The resulting mixture was filtered, the filter cake was washed with MeCN (3×3 mL). The filtrate was concentrated under reduced pressure, and added DCM (1 mL) to the concentrated filtrate. The system was purified by trituration with MTBE (3 mL). This resulted in 2-{2-chloro-3,3-dicyclohexyl-1,1-dimethylbenzo[d]1-aza-3-phospha-2-auracyclopentan-2-yl}-6-methoxybenzenesulfonyl fluoride and 2-{3,3-dicyclohexyl-2-iodo-1,1-dimethylbenzo[d]1-aza-3-phospha-2-auracyclopentan-2-yl}-6-methoxybenzenesulfonyl fluoride (130 mg, 32.71% yield, 98.1% purity) as a yellow solid. LCMS:(ES, m/z): [M]⁺=738.1.

¹H NMR (300 MHz, DMSO-d₆, ppm) δ 8.40 (dd, J=8.5, 4.3 Hz, 1H), 8.05 (t, J=9.0 Hz, 2H), 7.94-7.71 (m, 2H), 7.41 (dd, J=29.2, 8.5 Hz, 1H), 7.09-6.97 (m, 1H), 4.05 (d, J=2.2 Hz, 3H), 3.95-3.45 (m, 6H), 3.28-3.05 (m, 1H), 2.78 (s, 1H), 2.73 (s, 1H), 2.34-2.11 (m, 1H), 2.10-1.92 (m, 1H), 1.78 (s, 2H), 1.70-1.39 (m, 9H), 1.25 (s, 3H), 1.01 (dd, J=34.3, 18.5 Hz, 3H), 0.38 (s, 1H).

### Example 29. Synthesis of SFY—Au-3-1

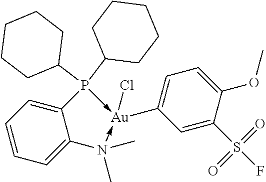

Step 1: To a stirred mixture of 5-iodo-2-methoxyaniline (1 g, 4.015 mmol, 1 equiv) and NaNO₂ (0.28 g, 4.015 mmol, 1 equiv) in EtOH (2 mL) were added Tetrafluoroboric acid (40% in H₂O) (1.76 g, 20.075 mmol, 5 equiv) and H₂O (1:1) dropwise at 0° C. The reaction was monitored by LCMS. The precipitated solids were collected by filtration and washed with EtOH (2 mL) (2×1 mL). This resulted in 5-iodo-2-methoxybenzenediazonium (800 mg, 76.33% yield) as a white solid.

Step 2: To a stirred mixture of 5-iodo-2-methoxybenzenediazonium (800 mg, 3.065 mmol, 1 equiv) and N-(benzenesulfonyl)-S-phenylfluoranesulfonamido (966.37 mg, 3.065 mmol, 1 equiv) in ACN (4.5 mL) was added potassium metabisulfite (1362.60 mg, 6.130 mmol, 2 equiv) in portions at room temperature under nitrogen atmosphere. To the above mixture was added AcOH (0.3 mL) and H₂O (75 uL) in portions over 5 min at room temperature. The resulting mixture was stirred at room temperature for additional 6 h. The reaction was monitored by LCMS. The resulting mixture was extracted with CH₂Cl₂ (3×1 mL). The combined organic layers were washed with water (3×1 mL), dried over anhydrous Na₂SO₄. After filtration, the filtrate was concentrated under reduced pressure. This resulted in 5-iodo-2-methoxybenzenesulfonic acid (900 mg, 93.50% yield) as a white solid.

Step 3: A solution of 5-iodo-2-methoxybenzenesulfonic acid (900 mg, 2.865 mmol, 1 equiv) in DCM (4 mL) was treated with DAST (92.38 mg, 0.573 mmol, 0.2 equiv) at room temperature for 1 h under nitrogen atmosphere. The reaction was monitored by LCMS. The reaction was quenched with ice water at room temperature. The combined organic layers were washed with water (3×1 mL), dried over anhydrous Na₂SO₄. After filtration, the filtrate was concentrated under reduced pressure. The residue was purified by Prep-TLC (PE/EA 5:1) to afford 5-iodo-2-methoxybenzenesulfonyl fluoride (45 mg, 4.97% yield) as a yellow solid.

Step 4: Into a 40 mL sealed tube were added Silver Hexafluoroantimonate(V) (54.36 mg, 0.158 mmol, 1 equiv) and DCM (10 mL) at 0° C. To the above mixture was added 2-[(chloroaurio)dicyclohexyl-lambda5-phosphanyl]-N,N-dimethylaniline (87.14 mg, 0.158 mmol, 1 equiv) and 5-iodo-2-methoxybenzenesulfonyl fluoride (50 mg, 0.158 mmol, 1.00 equiv) dropwise over 3 min at 0° C. The resulting mixture was stirred at room temperature for additional overnight. The resulting mixture was filtered, the filter cake was washed with MeCN (3×3 mL). The filtrate was concentrated under reduced pressure, and added DCM (0.6 mL) to the concentrated filtrate. The system was purified by trituration with MTBE (1.8 mL). This resulted in 5-{2-chloro-3,3-dicyclohexyl-1,1-dimethylbenzo[d]1-aza-3-phospha-2-auracyclopentan-2-yl}-2-methoxybenzenesulfonyl fluoride (35 mg, 29.94% yield, 75.7% purity) as a grey solid. LCMS:(ES, m/z): [M]⁺=738.1.

¹H NMR (300 MHz, DMSO-d₆, ppm) δ 8.46-8.29 (m, 1H), 8.22-7.85 (m, 3H), 7.84-7.58 (m, 2H), 7.39 (d, J=8.4 Hz, 1H), 4.18-3.82 (m, 3H), 3.52 (d, J=90.9 Hz, 6H), 3.03 (s, 2H), 2.04 (s, 2H), 1.66 (s, 10H), 1.36 (s, 4H), 1.04 (s, 2H), 0.62 (s, 2H).

### Example 30. Synthesis of AU-PM-8

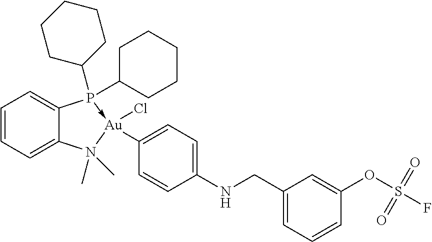

Step 1: To a stirred mixture of benzenamine, 4-iodo- (500 mg, 2.283 mmol, 1 equiv) and 3-formylphenyl sulfurofluoridate (699.13 mg, 3.425 mmol, 1.5 equiv) in DCM (10 mL) was added AcOH (137.09 mg, 2.283 mmol, 1 equiv) and STAB (967.65 mg, 4.566 mmol, 2 equiv) in portions at 0° C. The resulting mixture was stirred at room temperature for 1 h. The resulting mixture was extracted with CH₂Cl₂ (3×10 mL). The combined organic layers were washed with water (3×10 mL), dried over anhydrous Na₂SO₄. After filtration, the filtrate was concentrated under reduced pressure. The residue was purified by reversed-phase flash chromatography with the following conditions: column, C18 silica gel; mobile phase, MeCN in Water (0.1% FA), 10% to 95% gradient in 10 min; detector, UV 254 nm. This resulted in 3-{[(4-iodophenyl) amino]methyl}phenyl sulfurofluoridate (300 mg, 32.27% yield) as a white solid.

Step 2: To a stirred solution of 3-{[(4-iodophenyl) amino]methyl}phenyl sulfurofluoridate (100 mg, 0.246 mmol, 1 equiv) and (chloroaurio)dicyclohexyl[2-(dimethylamino) phenyl]-lambda5-phosphanyl (135.04 mg, 0.246 mmol, 1 equiv) in DCM (3 mL) were added Silver Hexafluoroantimonate(V) (84.39 mg, 0.246 mmol, 1 equiv) in portions at 0° C. under nitrogen atmosphere. The resulting mixture was stirred at room temperature for additional 2 h. The reaction was monitored by LCMS. The resulting mixture was filtered, the filter cake was washed with DCM (3×3 mL). The filtrate was concentrated under reduced pressure, and added DCM (1 mL) to the concentrated filtrate. The system was purified by trituration with MTBE (4 mL). This resulted in [chloro({4-[({3-[(fluorosulfonyl) oxy]phenyl}methyl) amino]phenyl}) aurio]dicyclohexyl[2-(dimethylamino) phenyl]-lambda5-phosphanyl (70.2 mg, 34.43% yield, 90.8% purity) as a dark green solid. LCMS:(ES, m/z): [M]⁺=829.2.

¹H NMR (300 MHz, DMSO-d₆, ppm) δ 8.31 (dd, J=8.4, 4.4 Hz, 1H), 8.10-7.96 (m, 2H), 7.81-7.71 (m, 1H), 7.64-7.42 (m, 4H), 7.00 (d, J=8.4 Hz, 2H), 6.63 (d, J=8.5 Hz, 2H), 4.41 (s, 2H), 2.87 (d, J=11.4 Hz, 2H), 1.84-1.46 (m, 12H), 1.28 (dd, J=20.8, 11.1 Hz, 4H), 0.97 (t, J=12.8 Hz, 2H), 0.58 (s, 2H).

### Example 31. Synthesis of Au—PP-8

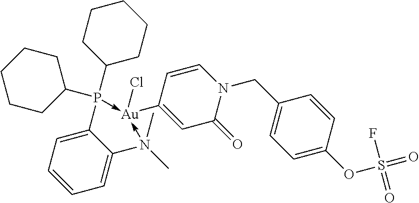

Step 1: To a stirred solution of 4-iodo-1H-pyridin-2-one (500 mg, 2.262 mmol, 1 equiv) and K2CO3 (469.03 mg, 3.393 mmol, 1.5 equiv) in DMF (5 mL) were added 4-methoxybenzyl chloride (425.19 mg, 2.714 mmol, 1.2 equiv) dropwise at 0° C. under nitrogen atmosphere. The resulting mixture was stirred at room temperature for additional 3 h. The resulting mixture was diluted with 5 mL H₂O and extracted with EtOAc (5 mL×3). The combined organic layers were washed with brine (5 mL), dried over anhydrous Na₂SO₄. After filtration, the filtrate was concentrated under reduced pressure. The residue was purified by silica gel column chromatography, eluted with PE/EA (3:1) to afford 4-iodo-1-[(4-methoxyphenyl) methyl]pyridin-2-one (500 mg, 64.78% yield) as a light yellow oil.

Step 2: To a stirred solution of 4-iodo-1-[(4-methoxyphenyl) methyl]pyridin-2-one (500 mg, 1.466 mmol, 1 equiv) in DCM (3 mL) were added Boron tribromide 1M solution in methylene chloride (6 mL) dropwise at 0° C. under nitrogen atmosphere. The resulting mixture was stirred at 0° C. for additional 1 h. The reaction was quenched with ice water at 0° C. The resulting mixture was stirred at room temperature for additional 3 h. The resulting mixture was diluted with 10 mL H₂O and extracted with DCM (10 mL×3). The combined organic layers were washed with brine (10 mL), dried over anhydrous Na₂SO₄. After filtration, the filtrate was concentrated under reduced pressure. This resulted in 1-[(4-hydroxyphenyl) methyl]-4-iodopyridin-2-one (300 mg, 62.57% yield), as a light brown solid. The crude product was used in the next step directly without further purification.

Step 3: To a stirred solution of 1-[(4-hydroxyphenyl) methyl]-4-iodopyridin-2-one (300 mg, 0.917 mmol, 1 equiv) and 3-(fluorosulfonyl)-1,2-dimethylimidazol-1-ium triflate (451.55 mg, 1.376 mmol, 1.5 equiv) in MeCN (5 mL) was added TEA (185.61 mg, 1.834 mmol, 2 equiv) dropwise at 0° C. under nitrogen atmosphere. The resulting mixture was stirred at room temperature for additional 30 min. The resulting mixture was concentrated under reduced pressure. The residue was purified by reversed-phase flash chromatography with the following conditions: column, C18 silica gel; mobile phase, MeCN in Water (0.1% FA), 10% to 90% gradient in 10 min; detector, UV 254 nm. This resulted in 4-[(4-iodo-2-oxopyridin-1-yl) methyl]phenyl sulfurofluoridate (170 mg, 45.30% yield) as a light brown solid.

Step 4: To a stirred solution of Silver Hexafluoroantimonate(V) (142.77 mg, 0.415 mmol, 1 equiv) in DCM (3 mL) at 0° C. under nitrogen atmosphere. To the above mixture was added 4-[(4-iodo-2-oxopyridin-1-yl)methyl]phenyl sulfurofluoridate (170 mg, 0.415 mmol, 1 equiv) and in portions at 0° C. The resulting mixture was stirred at room temperature for additional overnight. The resulting mixture was filtered, the filter cake was washed with DCM (1 mL×3). The filtrate was concentrated under reduced pressure, and added DCM (1 mL) to the concentrated filtrate. The system was purified by trituration with MTBE (4 mL). This resulted in 2-chloro-3,3-dicyclohexyl-2-[1-({4-[(fluorosulfonyl)oxy]phenyl}methyl)-2-oxopyridin-4-yl]-1,1-dimethyl-2H,3H-3lambda5-benzo[d]1-aza-3lambda5-phospha-2-auracyclopentan-1-ium-3-ylium-2,2-diuide (55 mg, 15.91% yield, 99.9% purity) as a light brown solid. LCMS:(ES, m/z): [M]⁺=831.1.

¹H NMR (300 MHz, DMSO-d₆, ppm) δ 8.35 (dd, J=8.4, 4.3 Hz, 1H), 8.05 (q, J=8.9, 8.2 Hz, 2H), 7.81 (t, J=5.9 Hz, 2H), 7.63 (d, J=8.4 Hz, 2H), 7.49 (d, J=8.7 Hz, 2H), 6.60 (d, J=7.9 Hz, 2H), 5.16 (d, J=26.4 Hz, 2H), 3.43 (s, 6H), 3.02 (s, 2H), 1.98 (s, 2H), 1.83-1.47 (m, 10H), 1.33 (d, J=12.3 Hz, 4H), 1.03 (t, J=12.5 Hz, 2H), 0.88 (d, J=14.8 Hz, 2H).

### Example 32. Synthesis of AU-PP-1

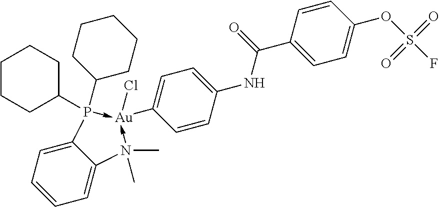

Step 1: To a stirred solution of 4-[(fluorosulfonyl)oxy]benzoic acid (552.88 mg, 2.511 mmol, 1.1 equiv), DIEA (885.15 mg, 6.849 mmol, 3 equiv) and HATU (1302.03 mg, 3.425 mmol, 1.5 equiv) in DCM (12 mL) were added 4-iodo-benzenamine (500 mg, 2.283 mmol, 1.00 equiv) dropwise at 0° C. The resulting mixture was stirred at 25° C. for additional 1 h. The reaction was monitored by LCMS. The resulting mixture was extracted with DCM (3×5 mL). The combined organic layers were dried over anhydrous Na₂SO₄. After filtration, the filtrate was concentrated under reduced pressure. The residue was purified by reversed-phase flash chromatography with the following conditions: column, C18 silica gel; mobile phase, MeCN in Water (0.1% FA), 10% to 60% gradient in 12 min; detector, UV 254 nm. This resulted in 4-[(4-iodophenyl)carbamoyl]phenyl sulfurofluoridate (300 mg, 31.20% yield) as a white solid.

Step 2: To a stirred mixture of AgSbF₆ (163.17 mg, 0.475 mmol, 1 equiv) in DCM (8 mL) were added 4-[(4-iodophenyl)carbamoyl]phenyl sulfurofluoridate (200 mg, 0.475 mmol, 1 equiv) and (chloroaurio)dicyclohexyl[2-(dimethylamino)phenyl]-lambda5-phosphanyl (261.11 mg, 0.475 mmol, 1 equiv) in portions at −20° C. The resulting mixture was stirred at 25° C. for additional 2 h. The resulting mixture was filtered, the filter cake was washed with MeCN. The filtrate was concentrated under reduced pressure. The residue was purified by trituration with Et₂O (2 mL). This resulted in product (100 mg, 24.95% yield) as a light grey solid.

LCMS: ((ES, m/z): [M]⁺=843.0.

¹H NMR (300 MHz, DMSO-d₆, ppm) δ 10.54 (s, 1H), 8.35 (dd, J=8.4, 4.4 Hz, 1H), 8.20-8.12 (m, 2H), 8.12-7.97 (m, 2H), 7.81 (dd, J=9.3, 2.8 Hz, 5H), 7.42 (d, J=8.5 Hz, 2H), 3.44 (s, 6H), 2.95 (q, J=11.2 Hz, 2H), 1.89 (s, 2H), 1.81-1.47 (m, 10H), 1.47-1.19 (m, 4H), 1.07 (dd, J=9.8, 6.0 Hz, 2H), 0.76-0.52 (m, 2H).

### Example 33. Synthesis of Au-Pyr-1

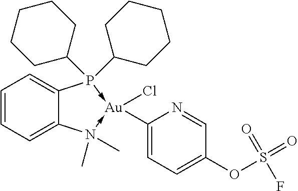

Step 1: A solution of 6-iodopyridin-3-ol (500 mg, 2.26 mmol, 1 equiv) in ACN (3 mL) was treated with 3-(fluorosulfonyl)-1,2-dimethylimidazol-1-ium triflate (1113.99 mg, 3.39 mmol, 1.5 equiv) at room temperature for 3 min followed by the addition of TEA (457.89 mg, 4.524 mmol, 2 equiv) dropwise at 0° C. The reaction was monitored by LCMS. The resulting mixture was concentrated under reduced pressure. The crude product was purified by reverse phase flash to afford 6-iodopyridin-3-yl sulfurofluoridate (200 mg, 29.17% yield) as a yellow oil.

Step 2: To a stirred mixture of 2-[(chloroaurio)dicyclohexyl-lambda5-phosphanyl]-N,N-dimethylaniline (127.25 mg, 0.23 mmol, 1 equiv) in DCM were added 6-iodopyridin-3-yl sulfurofluoridate (70 mg, 0.23 mmol, 1 equiv) and Silver Hexafluoroantimonate(V) (79.37 mg, 0.23 mmol, 1 equiv) in portions at 0° C. under nitrogen atmosphere. The reaction was monitored by LCMS. The resulting mixture was filtered, the filter cake was washed with DCM (3×1 mL). The filtrate was concentrated under reduced pressure. The residue was purified by trituration with Et₂O (3 mL). The precipitated solids were collected by filtration and washed with Et₂O (3×1 mL). This resulted in 6-{2-chloro-3,3-dicyclohexyl-1,1-dimethylbenzo[d]1-aza-3-phospha-2-auracyclopentan-2-yl}pyridin-3-yl sulfurofluoridate (65 mg, 38.76% yield, 97.5% purity) as a grey solid. LCMS:(ES, m/z): [M]⁺=725.1.

¹H NMR (300 MHz, DMSO-d₆, ppm) δ 9.00 (d, J=3.0 Hz, 1H), 8.34 (dd, J=8.6, 4.4 Hz, 1H), 8.18 (dd, J=8.8, 3.0 Hz, 1H), 8.11-7.96 (m, 3H), 7.77 (tt, J=7.9, 4.0 Hz, 1H), 3.62 (s, 6H), 3.04 (q, J=11.3 Hz, 2H), 1.83 (s, 2H), 1.75-1.45 (m, 10H), 1.31 (t, J=13.2 Hz, 4H), 0.98 (q, J=12.8 Hz, 2H), 0.42-0.25 (m, 2H).

### Example 34. Synthesis of Au-01

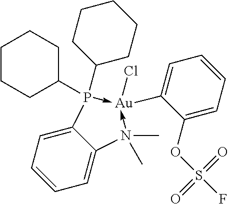

Step 1: Into a 40 mL sealed tube were added dicyclohexylphosphane (1.5 g, 7.56 mmol, 1.00 equiv), 2-iodo-N,N-dimethylaniline (1.96 g, 7.94 mmol, 1.05 equiv), t-BuONa (1.09 g, 11.34 mmol, 1.5 equiv), 1,1′-bis(diisopropylphosphino)ferrocene (0.10 g, 0.22 mmol, 0.03 equiv), Pd(OAc)2 (0.04 g, 0.18 mmol, 0.025 equiv) and toluene (20 mL) at room temperature. The resulting mixture was stirred at 100° C. for overnight under nitrogen atmosphere. The mixture was allowed to cool down to room temperature. The resulting mixture was filtered, the filter cake was washed with MeCN (3×5 mL). The filtrate was concentrated under reduced pressure. The residue was purified by silica gel column chromatography, eluted with PE/EA (5:1) to afford 2-(dicyclohexylphosphanyl)-N,N-dimethylaniline (860 mg, 35.81% yield, 95% purity) as a yellow solid.

Step 2: Into a 40 mL sealed tube were added 2-(dicyclohexylphosphanyl)-N,N-dimethylaniline (905.33 mg, 2.85 mmol, 1 equiv) and DCM (10 mL) at room temperature. To the above mixture was added (chloroaurio)dimethyl-lambda3-sulfane (840 mg, 2.85 mmol, 1 equiv) dropwise over 3 min at −20° C. The resulting mixture was stirred at room temperature for additional overnight. The resulting mixture was filtered, the filter cake was washed with DCM (3×3 mL). The filtrate was concentrated under reduced pressure. The residue was purified by trituration with Et₂O (10 mL). This resulted in 2-[(chloroaurio)dicyclohexyl-lambda5-phosphanyl]-N,N-dimethylaniline (1 g, 63.65% yield, 95% purity) as a grey solid.

Step 3: Into a 40 mL sealed tube were added 2-iodophenol (500 mg, 2.27 mmol, 1.00 equiv), MeCN (5 mL) and 3-(fluorosulfonyl)-1,2-dimethylimidazol-1-ium triflate (969.79 mg, 2.95 mmol, 1.3 equiv) at room temperature. To the above mixture was added Et3N (689.92 mg, 6.82 mmol, 3 equiv) dropwise over 3 min at 0° C. The resulting mixture was stirred at 0° C. for additional 1 h. The residue was purified by silica gel column chromatography, eluted with PE/EA (1:1) to afford 2-iodophenyl sulfurofluoridate (600 mg, 87.40% yield, 95% purity) as a yellow oil.

Step 4: Into a 40 mL sealed tube were added Silver Hexafluoroantimonate(V) (155.94 mg, 0.45 mmol, 1 equiv) and DCM (10 mL) at −10° C. To the above mixture was added 2-[(chloroaurio)dicyclohexyl-lambda5-phosphanyl]-N,N-dimethylaniline (250 mg, 0.45 mmol, 1 equiv) and 2-iodophenyl sulfurofluoridate (150.79 mg, 0.50 mmol, 1.1 equiv) dropwise over 2 min at 0° C. The resulting mixture was stirred at room temperature for additional overnight. The resulting mixture was filtered, the filter cake was washed with MeCN (3×5 mL). The filtrate was concentrated under reduced pressure. The residue was purified by trituration with 1,4-dioxane (10 mL). The precipitated solids were collected by filtration and washed with 1,4-dioxane (3×5 mL). This resulted in 2-{2-chloro-3,3-dicyclohexyl-1,1-dimethylbenzo[d]1-aza-3-phospha-2-auracyclopentan-2-yl}phenyl sulfurofluoridate (95.5 mg, 29.02% yield, 96.2% purity) as a grey solid. LCMS:(ES, m/z): [M]⁺=724.1.

¹H NMR (300 MHz, DMSO-d₆) δ 8.40 (dd, J=8.6, 4.3 Hz, 1H), 8.14-8.01 (m, 2H), 7.81 (ddd, J=14.1, 7.2, 2.3 Hz, 2H), 7.69-7.66 (m, 1H), 7.63-7.53 (m, 2H), 3.59 (s, 3H), 3.48 (s, 3H), 3.12-2.84 (m, 2H), 1.64-1.89 (m, 11H), 1.28-1.41 (m, 5H), 1.07 (m, 2H), 0.53 (m, 2H).

### Example 35. Synthesis of Au-02

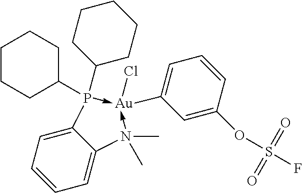

Step 1: Into a 40 mL sealed tube were added 3-iodophenol (300 mg, 1.36 mmol, 1.00 equiv), MeCN (10 mL) and 1-(fluorosulfonyl)-2,3-dimethyl-1H-imidazol-3-ium trifluoromethanesulfonate (581.87 mg, 1.77 mmol, 1.30 equiv) at room temperature. To the above mixture was added Et₃N (413.95 mg, 4.09 mmol, 3.00 equiv) dropwise over 3 min at −10° C. The resulting mixture was stirred at −10° C. for additional 1 h. The residue was purified by silica gel column chromatography, eluted with PE/EA (8:1) to afford 3-iodophenyl sulfurofluoridate (200 mg, 48.56% yield, 95% purity) as a yellow oil.

Step 2: Into a 40 mL sealed tube were added Silver Hexafluoroantimonate(V) (124.75 mg, 0.36 mmol, 1.00 equiv) and DCM (10 mL) at −10° C. To the above mixture was added 2-[(chloroaurio)dicyclohexyl-lambda5-phosphanyl]-N,N-dimethylaniline (200 mg, 0.36 mmol, 1.00 equiv) and 3-iodophenyl sulfurofluoridate (120.63 mg, 0.40 mmol, 1.10 equiv) dropwise over 2 min at −10° C. The resulting mixture was stirred at room temperature for additional overnight. The resulting mixture was filtered, the filter cake was washed with MeCN (3×3 mL). The filtrate was concentrated under reduced pressure. The residue was purified by trituration with DCM (10 mL). The precipitated solids were collected by filtration and washed with DCM (10 mL) (3×3 mL). This resulted in 3-{2-chloro-3,3-dicyclohexyl-1,1-dimethyl-3H-1lambda4,3lambda5-benzo[d]1lambda4-aza-3lambda5-phospha-2-auracyclopentan-2-yl}phenyl sulfurofluoridate (80.3 mg, 30.46% yield, 93.2% purity) as a grey solid. LCMS:(ES, m/z): [M]⁺=724.0.

¹H NMR (300 MHz, DMSO-d₆, ppm) δ 8.36 (dd, J=8.5, 4.4 Hz, 1H), 8.12-7.97 (m, 2H), 7.86-7.72 (m, 2H), 7.61 (d, J=3.2 Hz, 3H), 3.46 (s, 6H), 2.92 (d, J=11.1 Hz, 2H), 2.72 (s, 2H), 1.87 (s, 2H), 1.63 (s, 10H), 1.43-1.20 (m, 4H), 1.07 (d, J=12.6 Hz, 2H), 0.56 (s, 2H).

### Example 36. Synthesis of Au-03

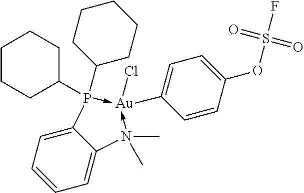

Step 1: Into a 40 mL sealed tube were added 4-iodophenol (300 mg, 1.36 mmol, 1 equiv), MeCN (10 mL) and 1-(fluorosulfonyl)-2,3-dimethyl-1H-imidazol-3-ium trifluoromethane sulfonate (581.87 mg, 1.77 mmol, 1.3 equiv) at room temperature. To the above mixture was added Et3N (413.95 mg, 4.092 mmol, 3 equiv) dropwise over 3 min at −10° C. The resulting mixture was stirred at −10° C. for additional 1 h. The residue was purified by silica gel column chromatography, eluted with PE/EA (8:1) to afford 4-iodophenyl sulfurofluoridate (200 mg, 48.56% yield, 95% purity) as a yellow oil.

Step 2: Into a 40 mL sealed tube were added Silver Hexafluoroantimonate(V) (124.75 mg, 0.36 mmol, 1 equiv) and DCM (10 mL) at −10° C. To the above mixture was added 2-[(chloroaurio)dicyclohexyl-lambda5-phosphanyl]-N,N-dimethylaniline (200 mg, 0.36 mmol, 1 equiv) and 4-iodophenyl sulfurofluoridate (120.63 mg, 0.399 mmol, 1.1 equiv) dropwise over 3 min at −10° C. The resulting mixture was stirred at room temperature for additional overnight. The resulting mixture was filtered, the filter cake was washed with MeCN (3×3 mL). The filtrate was concentrated under reduced pressure. The residue was purified by trituration with MTBE (20 mL). The precipitated solids were collected by filtration and washed with MTBE (3×3 mL). This resulted in 4-{2-chloro-3,3-dicyclohexyl-1,1-dimethylbenzo[d]1-aza-3-phospha-2-auracyclopentan-2-yl}phenyl sulfurofluoridate (93.2 mg, 35.41% yield, 97.0% purity) as a white solid. LCMS:(ES, m/z): [M]⁺=724.1.

¹H NMR (300 MHz, DMSO-d₆) δ 8.36 (dd, J=8.5, 4.4 Hz, 1H), 8.05 (q, J=7.8, 7.3 Hz, 2H), 7.78 (td, J=7.4, 2.4 Hz, 1H), 7.76-7.63 (m, 4H), 3.46 (s, 6H), 2.95 (q, J=11.7 Hz, 2H), 1.87 (m, 2H), 1.60 (m, 10H), 1.31 (dt, J=23.9, 12.0 Hz, 4H), 1.14-0.95 (m, 2H), 0.46 (m, 2H).

### Example 37. Synthesis of Au-04

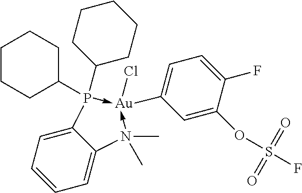

Step 1: Into a mL sealed tube were added 2-fluoro-5-iodophenol (800 mg, 3.36 mmol, 1 equiv), MeCN (10 mL) and 1-(fluorosulfonyl)-2,3-dimethyl-1H-imidazol-3-ium trifluoromethanesulfonate (1434.37 mg, 4.36 mmol, 1.3 equiv) at room temperature. To the above mixture was added Et3N (1020.43 mg, 10.08 mmol, 3 equiv) dropwise over 3 min at −10° C. The resulting mixture was stirred at −10° C. for additional 1 h. The residue was purified by silica gel column chromatography, eluted with PE/EA (8:1) to afford 2-fluoro-5-iodophenyl sulfurofluoridate (460 mg, 42.76% yield, 99% purity) as a yellow oil.

Step 2: Into a 40 mL sealed tube were added silver(I) hexafluorophosphate(V) (79.00 mg, 0.31 mmol, 1 equiv) and DCM (10 mL) at room temperature. To the above mixture was added 2-[(chloroaurio)dicyclohexyl-lambda5-phosphanyl]-N,N-dimethylaniline (172.12 mg, 0.31 mmol, 1 equiv) and 2-fluoro-5-iodophenyl sulfurofluoridate (110 mg, 0.34 mmol, 1.1 equiv) dropwise over 3 min at −10° C. The resulting mixture was stirred at room temperature for additional overnight. The resulting mixture was filtered, the filter cake was washed with MeCN (3×3 mL). The filtrate was concentrated under reduced pressure. The residue was purified by trituration with MTBE (10 mL). The precipitated solids were collected by filtration and washed with MTBE (3×3 mL). This resulted in 5-{2-chloro-3,3-dicyclohexyl-1,1-dimethylbenzo[d]1-aza-3-phospha-2-auracyclopentan-2-yl}-2-fluorophenyl sulfurofluoridate (141.4 mg, 60.91% yield, 96.5% purity) as a grey solid. LCMS:(ES, m/z): [M]⁺=742.0.

¹H NMR (300 MHz, DMSO-d₆) δ 8.37 (dd, J=8.5, 4.3 Hz, 1H), 8.14-7.98 (m, 3H), 7.85-7.70 (m, 2H), 7.64 (ddd, J=8.8, 4.8, 1.9 Hz, 1H), 3.48 (s, 6H), 2.93 (m, 2H), 1.91 (m, 2H), 1.64 (m, 10H), 1.32 (m, 4H), 1.07 (m, 2H), 0.61 (m, 2H).

### Example 38. Synthesis of Au-05

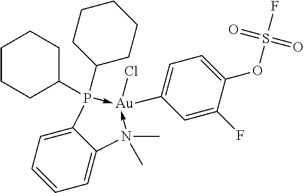

Step 1: Into a mL sealed tube were added O-fluorophenol (300 mg, 2.67 mmol, 1 equiv), MeCN (5 mL) and 1-(fluorosulfonyl)-2,3-dimethyl-1H-imidazol-3-ium trifluoromethanesulfonate (1141.96 mg, 3.47 mmol, 1.3 equiv) at room temperature. To the above mixture was added Et3N (812.41 mg, 8.02 mmol, 3 equiv) dropwise over 3 min at −10° C. The resulting mixture was stirred at −10° C. for additional 1 h. The residue was purified by silica gel column chromatography, eluted with PE/EA (8:1) to afford 2-fluoro-4-iodophenyl sulfurofluoridate (110 mg, 12.84% yield, 99% purity) as a yellow oil.

Step 2: Into a 40 mL sealed tube were added Silver Hexafluoroantimonate(V) (214.73 mg, 0.62 mmol, 1 equiv) and DCM (10 mL) at −10° C. To the above mixture was added 2-fluoro-4-iodophenyl sulfurofluoridate (200 mg, 0.62 mmol, 1 equiv) and 2-[(chloroaurio)dicyclohexyl-lambda5-phosphanyl]-N,N-dimethylaniline (378.67 mg, 0.68 mmol, 1.1 equiv) dropwise over 3 min at −10° C. The resulting mixture was stirred at room temperature for additional overnight. The resulting mixture was filtered, the filter cake was washed with MeCN (3×3 mL). The filtrate was concentrated under reduced pressure. The residue was purified by trituration with MTBE (10 mL). The precipitated solids were collected by filtration and washed with DCM (10 mL) (3×3 mL). This resulted in 4-{2-chloro-3,3-dicyclohexyl-1,1-dimethylbenzo[d]1-aza-3-phospha-2-auracyclopentan-2-yl}-2-fluorophenyl sulfurofluoridate (57.8 mg, 12.45% yield, 92.4% purity) as a grey solid. LCMS:(ES, m/z): [M]⁺=742.0.

¹H NMR (300 MHz, DMSO-d₆) δ 8.36 (dd, J=8.6, 4.4 Hz, 1H), 8.11-7.98 (m, 2H), 7.97-7.84 (m, 2H), 7.78 (td, J=7.5, 2.5 Hz, 1H), 7.51 (d, J=8.8 Hz, 1H), 3.46 (s, 6H), 2.95 (m, 2H), 1.91 (s, 2H), 1.63 (m, 10H), 1.39-1.25 (m, 4H), 1.12-0.93 (m, 2H), 0.48 (m, 2H).

### Example 39. Synthesis of Au-8

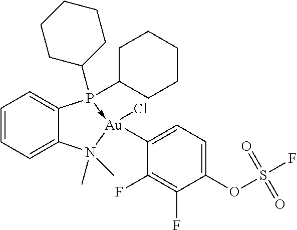

Step 1: Into a 40 mL sealed tube were added 2,3-difluoro-4-iodophenol (500 mg, 1.95 mmol, 1.00 equiv), MeCN (5 mL) and 3-(fluorosulfonyl)-1,2-dimethylimidazol-1-ium triflate (833.48 mg, 2.54 mmol, 1.3 equiv) at room temperature. To the above mixture was added Et3N (592.95 mg, 5.86 mmol, 3 equiv) dropwise over 3 min at 0° C. The resulting mixture was stirred at ° C. for additional 1 h. The residue was purified by reversed-phase flash chromatography with the following conditions: column, C18 silica gel; mobile phase, MeCN in Water (0.1% FA), 10% to 50% gradient in 10 min; detector, UV 254 nm. This resulted in 2,3-difluoro-4-iodophenyl sulfurofluoridate (60 mg, 9.09% yield, 95% purity) as a yellow oil.

Step 2: Into a 8 mL sealed tube were added Silver Hexafluoroantimonate(V) (60.99 mg, 0.18 mmol, 1 equiv) and DCM (3 mL) at room temperature. To the above mixture was added 2-[(chloroaurio)dicyclohexyl-lambda5-phosphanyl]-N,N-dimethylaniline (97.78 mg, 0.18 mmol, 1 equiv) and 2,3-difluoro-4-iodophenyl sulfurofluoridate (60 mg, 0.18 mmol, 1.00 equiv) dropwise over 3 min at 0° C. The resulting mixture was stirred at 0° C. for additional 4 h. The resulting mixture was filtered, the filter cake was washed with MeOH (3×3 mL). The filtrate was concentrated under reduced pressure. The residue was purified by trituration with MTBE (3 mL). This resulted in 4-{2-chloro-3,3-dicyclohexyl-1,1-dimethylbenzo[d]1-aza-3-phospha-2-auracyclopentan-2-yl}-2,3-difluorophenyl sulfurofluoridate (63 mg, 46.64% yield, 96.5% purity) as a grey solid. LCMS:(ES, m/z): [M]⁺=760.1.

¹H NMR (300 MHz, DMSO-d₆) δ 8.41 (dd, J=8.5, 4.3 Hz, 1H), 8.08 (m, 2H), 7.92-7.76 (m, 2H), 7.69 (m, 1H), 3.56 (d, J=13.3 Hz, 6H), 3.08 (m, 2H), 2.04 (m, 1H), 1.92 (m, 1H), 1.65 (m, 9H), 1.40-1.27 (m, 5H), 0.98 (m, 2H), 0.55 (m, 2H).

### Example 40. Synthesis of Au-4-1-Me

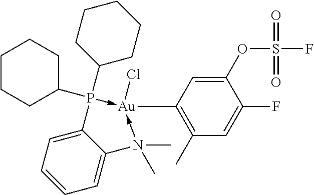

Step 1: To a stirred mixture of 2-fluoro-5-iodo-4-methylphenol (250 mg, 0.992 mmol, 1 equiv) and 3-(fluorosulfonyl)-1,2-dimethylimidazol-1-ium triflate (390.73 mg, 1.190 mmol, 1.2 equiv) in MeCN (5 mL) was added TEA (150.57 mg, 1.488 mmol, 1.5 equiv) dropwise at 0° C. The resulting mixture was concentrated under reduced pressure. The residue was purified by reversed-phase flash chromatography with the following conditions: column, C18 silica gel; mobile phase, MeCN in Water (0.1% FA), 10% to 95% gradient in 10 min; detector, UV 254 nm. This resulted in 2-fluoro-5-iodo-4-methylphenyl sulfurofluoridate (100 mg, 30.18% yield) as a yellow oil.

Step 2: To a stirred solution of 2-fluoro-5-iodo-4-methylphenyl sulfurofluoridate (100 mg, 0.299 mmol, 1 equiv) and 2-[(chloroaurio)dicyclohexyl-lambda5-phosphanyl]-N,N-dimethylaniline (164.59 mg, 0.299 mmol, 1 equiv) in DCM (3 mL) were added Silver Hexafluoroantimonate(V) (102.86 mg, 0.299 mmol, 1 equiv) in portions at room temperature under nitrogen atmosphere. The resulting mixture was stirred at room temperature for additional 2 h. The reaction was monitored by LCMS. The resulting mixture was filtered, the filter cake was washed with DCM (3×3 mL). The filtrate was concentrated under reduced pressure. The residue was purified by trituration with Methyl tert-butyl ether (3 mL). This resulted in 2-chloro-3,3-dicyclohexyl-2-{4-fluoro-5-[(fluorosulfonyl)oxy]-2-methylphenyl}-1,1-dimethyl-2H,3H-3lambda5-benzo[d]1-aza-3lambda5-phospha-2-auracyclopentan-1-ium-3-ylium-2,2-diuide (100 mg, 44.13% yield, 93.6% purity) as a grey solid. LCMS:(ES, m/z): [M]⁺=756.0.

¹H NMR (300 MHz, DMSO-d₆, ppm) δ 8.39 (dd, J=8.6, 4.4 Hz, 1H), 8.13-8.00 (m, 2H), 7.81 (td, J=7.4, 2.5 Hz, 1H), 7.70 (dd, J=19.4, 9.7 Hz, 2H), 3.50 (d, J=4.6 Hz, 6H), 3.25 (d, J=10.3 Hz, 1H), 2.74 (d, J=10.4 Hz, 1H), 2.63 (s, 3H), 2.37-2.23 (m, 1H), 2.01-1.87 (m, 1H), 1.82-1.24 (m, 14H), 1.17-0.95 (m, 2H), 0.81 (d, J=13.3 Hz, 1H), 0.40-0.21 (m, 1H).

### Example 41. Synthesis of Au-2-6-F

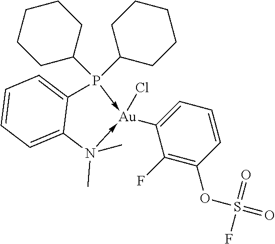

Step 1: To a stirred mixture of 2-fluoro-3-iodophenol (250 mg, 1.050 mmol, 1 equiv) and 3-(fluorosulfonyl)-1,2-dimethylimidazol-1-ium triflate (413.76 mg, 1.260 mmol, 1.2 equiv) in MeCN (5 mL) was added TEA (159.44 mg, 1.575 mmol, 1.5 equiv) in portions at 0° C. The resulting mixture was stirred at 0° C. for 30 min. The resulting mixture was concentrated under reduced pressure. The residue was purified by reversed-phase flash chromatography with the following conditions: column, C18 silica gel; mobile phase, MeCN in Water (0.1% FA), 10% to 95% gradient in 10 min; detector, UV 254 nm. This resulted in 2-fluoro-3-iodophenyl sulfurofluoridate (100 mg, 29.75% yield) as a white solid.

Step 2: To a stirred solution of 2-fluoro-3-iodophenyl sulfurofluoridate (50 mg, 0.156 mmol, 1 equiv) and (chloroaurio)dicyclohexyl[2-(dimethylamino)phenyl]-lambda5-phosphanyl (85.90 mg, 0.156 mmol, 1 equiv) in DCM (3 mL) were added Silver Hexafluoroantimonate(V) (53.68 mg, 0.156 mmol, 1 equiv) in portions at room temperature under nitrogen atmosphere. The resulting mixture was stirred at room temperature for additional 2 h. The reaction was monitored by LCMS. The resulting mixture was filtered, the filter cake was washed with DCM (3×3 mL). The filtrate was concentrated under reduced pressure. The residue was purified by trituration with Methyl tert-butyl ether (3 mL). This resulted in 3-[chloro({dicyclohexyl[2-(dimethylamino)phenyl]-lambda5-phosphanylidene})aurio]-2-fluorophenyl sulfurofluoridate (63 mg, 54.27% yield, 95.1% purity) as a grey solid. LCMS:(ES, m/z): [M]⁺=742.0.

¹H NMR (300 MHz, DMSO-d₆, ppm) δ 8.41 (dd, J=8.6, 4.3 Hz, 1H), 8.09 (dt, J=9.7, 7.1 Hz, 2H), 7.94-7.78 (m, 2H), 7.74 (ddd, J=8.2, 3.6, 1.4 Hz, 1H), 7.55 (dd, J=8.7, 7.5 Hz, 1H), 3.56 (d, J=16.3 Hz, 6H), 2.98 (s, 2H), 2.12-1.84 (m, 2H), 1.80-1.54 (m, 10H), 1.35 (dd, J=26.6, 13.7 Hz, 4H), 1.08 (s, 2H), 0.61 (s, 2H).

### Example 42. Synthesis of Au-MP-1

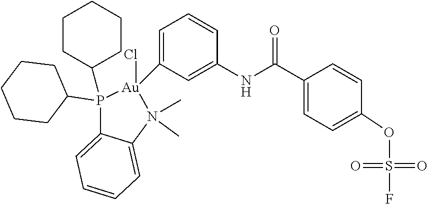

Step 1: To a stirred solution of 4-[(fluorosulfonyl)oxy]benzoic acid (552.88 mg, 2.511 mmol, 1.1 equiv), DIEA (885.15 mg, 6.849 mmol, 3 equiv) and HATU (1302.03 mg, 3.425 mmol, 1.5 equiv) in DCM (12 mL) were added 3-iodo-benzenamine (500 mg, 2.283 mmol, 1.00 equiv) dropwise at 0° C. The resulting mixture was stirred at 25° C. for additional 1 h. The reaction was monitored by LCMS. The resulting mixture was extracted with DCM (3×5 mL). The combined organic layers were dried over anhydrous Na₂SO₄. After filtration, the filtrate was concentrated under reduced pressure. The residue was purified by reversed-phase flash chromatography with the following conditions: column, C18 silica gel; mobile phase, MeCN in Water (0.1% FA), 10% to 60% gradient in 12 min; detector, UV 254 nm. This resulted in 4-[(3-iodophenyl)carbamoyl]phenyl sulfurofluoridate (300 mg, 31.20% yield) as a brown solid.

Step 2: To a stirred mixture of AgSbF₆ (163.17 mg, 0.475 mmol, 1 equiv) in DCM (8 mL) were added 4-[(3-iodophenyl)carbamoyl]phenyl sulfurofluoridate (200 mg, 0.475 mmol, 1 equiv) and (chloroaurio)dicyclohexyl[2-(dimethylamino)phenyl]-lambda5-phosphanyl (261.11 mg, 0.475 mmol, 1 equiv) in portions at −20° C. The resulting mixture was stirred at 25° C. for additional 2 h. The resulting mixture was filtered, the filter cake was washed with MeCN. The filtrate was concentrated under reduced pressure. The residue was purified by trituration with Et₂O (2 mL). This resulted in product (80 mg, 19.96% yield) as a light grey solid. LCMS: (ES, m/z): [M]⁺=843.0.

¹H NMR (300 MHz, DMSO-d₆, ppm) δ 10.61 (s, 1H), 8.36 (dd, J=8.5, 4.3 Hz, 1H), 8.19-7.98 (m, 5H), 7.88-7.73 (m, 3H), 7.43-7.32 (m, 2H), 7.31-7.21 (m, 1H), 3.44 (s, 6H), 3.21-3.02 (m, 2H), 1.95-1.81 (m, 2H), 1.80-1.50 (m, 10H), 1.47-1.15 (m, 4H), 1.15-0.92 (m, 2H), 0.59 (s, 2H).

### Example 43. Synthesis of Au-MP-3

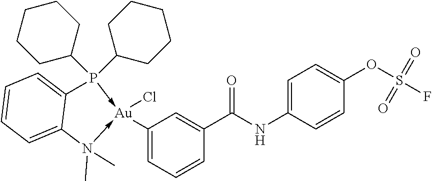

Step 1: A solution of tert-butyl N-(4-hydroxyphenyl) carbamate (500 mg, 2.39 mmol, 1 equiv) in MeCN (3 mL) was treated with 3-(fluorosulfonyl)-1,2-dimethylimidazol-1-ium triflate (1176.55 mg, 3.58 mmol, 1.5 equiv) at room temperature for 5 min under nitrogen atmosphere followed by the addition of TEA (483.61 mg, 4.78 mmol, 2 equiv) dropwise at 0° C. The reaction was monitored by LCMS. The resulting mixture was concentrated under reduced pressure. The residue product was purified by reverse phase flash to afford tert-butyl N-{4-[(fluorosulfonyl) oxy]phenyl}carbamate (700 mg, 64.57% yield) as a yellow solid.

Step 2: To the above mixture was added HCl in 1,4-dioxane (4.0 M) (4 mL, 1 equiv) dropwise over 1 min at room temperature. The resulting mixture was stirred at room temperature for additional 1 h. The reaction was monitored by LCMS. The resulting mixture was concentrated under reduced pressure. This resulted in 4-aminophenyl sulfurofluoridate hydrochloride (350 mg, 63.98% yield) as a white solid. The crude product was used in the next step directly without further purification.

Step 3 To a stirred mixture of 4-aminophenyl sulfurofluoridate hydrochloride (350 mg, 1.53 mmol, 1 equiv) and 3-iodobenzoic acid (381.35 mg, 1.53 mmol, 1 equiv) in DMF (3 mL) were added HATU (876.96 mg, 2.30 mmol, 1.5 equiv) and DIEA (596.18 mg, 4.61 mmol, 3 equiv) dropwise at room temperature. The reaction was monitored by LCMS. The resulting mixture was extracted with EtOAc (3×2 mL). The combined organic layers were washed with water (3×2 mL), dried over anhydrous Na₂SO₄. After filtration, the filtrate was concentrated under reduced pressure. The residue was purified by silica gel column chromatography, eluted with PE/EA (5:1) to afford 4-(3-iodobenzamido) phenyl sulfurofluoridate (150 mg, 23.16% yield) as a white solid.

Step 4: To a stirred solution of 2-[(chloroaurio)dicyclohexyl-lambda5-phosphanyl]-N,N-dimethylaniline (91.56 mg, 0.16 mmol, 1 equiv) and 4-(3-iodobenzamido)phenyl sulfurofluoridate (70 mg, 0.16 mmol, 1.00 equiv) in DCM (5 mL) were added Silver Hexafluoroantimonate(V) (57.11 mg, 0.16 mmol, 1 equiv) in portions at 0° C. under nitrogen atmosphere. The resulting mixture was stirred at room temperature for additional 2 h. The resulting mixture was filtered, the filter cake was washed with DCM (5.00 mL) (3×2 mL). The filtrate was concentrated under reduced pressure. The residue was purified by trituration with ethyl ether (5 mL). This resulted in 4-(3-{2-chloro-3,3-dicyclohexyl-1,1-dimethylbenzo[d]1-aza-3-phospha-2-auracyclopentan-2-yl}benzamido) phenyl sulfurofluoridate (65 mg, 46.33% yield, 95.1% purity) as a grey solid. LCMS:(ES, m/z): [M]⁺=843.1.

¹H NMR (300 MHz, DMSO-d₆, ppm) δ 10.63 (s, 1H), 8.37 (dd, J=8.6, 4.3 Hz, 1H), 8.15-7.91 (m, 6H), 7.79 (d, J=6.7 Hz, 1H), 7.73 (d, J=8.0 Hz, 1H), 7.67-7.53 (m, 3H), 3.47 (s, 6H), 2.92 (d, J=11.0 Hz, 2H), 1.91 (s, 2H), 1.67 (d, J=24.9 Hz, 10H), 1.32 (dd, J=30.0, 13.6 Hz, 4H), 1.08 (t, J=12.6 Hz, 2H), 0.62 (s, 2H).

### Example 44. Synthesis of Au-MM-2

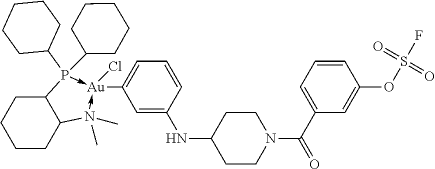

Step 1: A solution of benzenamine, 3-iodo- (1 g, 4.56 mmol, 1.00 equiv), tert-butyl 4-oxopiperidine-1-carboxylate (1.36 g, 6.84 mmol, 1.5 equiv) and HOAc (0.03 g, 0.45 mmol, 0.1 equiv) in DCM (10 mL) was stirred at room temperature for 3 min under nitrogen atmosphere. To the above mixture was added STAB (4.84 g, 22.83 mmol, 5 equiv) in portions over 3 min at 0° C. The resulting mixture was stirred at 0° C. for additional 1 h. The reaction was quenched by the addition of water (20 mL) at 0° C. The resulting mixture was extracted with CH₂Cl₂ (3×20 mL). The combined organic layers were washed with brine (1×20 mL), dried over anhydrous Na₂SO₄. After filtration, the filtrate was concentrated under reduced pressure. This resulted in crude tert-butyl 4-[(3-iodophenyl) amino]piperidine-1-carboxylate (2 g) as a yellow oil.

Step 2: A solution of tert-butyl 4-[(3-iodophenyl) amino]piperidine-1-carboxylate (2 g, 4.97 mmol, 1 equiv) in HCl in 1,4-dioxane (4.0 M) (20 mL) was stirred at room temperature for 2 h under nitrogen atmosphere. The resulting mixture was concentrated under reduced pressure. This resulted in N-(3-iodophenyl) piperidin-4-amine hydrochloride (1.5 g, Crude Product) as a white solid.

Step 3: A solution of N-(3-iodophenyl) piperidin-4-amine hydrochloride (1.5 g, 4.43 mmol, 1 equiv), 3-[(fluorosulfonyl) oxy]benzoic acid (0.98 g, 4.43 mmol, 1 equiv), HATU (3.37 g, 8.86 mmol, 2 equiv) and DIEA (1.72 g, 13.29 mmol, 3 equiv) in DCM (20 mL) was stirred at room temperature for 2 h under nitrogen atmosphere. The resulting mixture was concentrated under reduced pressure. The residue was purified by reversed-phase flash chromatography with the following conditions: column, C18 silica gel; mobile phase, MeCN in Water (0.1% FA), 10% to 50% gradient in 10 min; detector, UV 254 nm. This resulted in 3-{4-[(3-iodophenyl) amino]piperidine-1-carbonyl}phenyl sulfurofluoridate (150 mg, 6.71% yield, 95% purity) as a yellow oil.

Step 4: A solution of 2-[(chloroaurio)dicyclohexyl-lambda5-phosphanyl]-N, N-dimethylaniline (150 mg, 0.27 mmol, 1 equiv) and 3-{4-[(3-iodophenyl) amino]piperidine-1-carbonyl}phenyl sulfurofluoridate (151.05 mg, 0.29 mmol, 1.1 equiv) in DCM (10 mL) was stirred at room temperature for 3 min under nitrogen atmosphere. To the above mixture was added Silver Hexafluoroantimonate(V) (93.56 mg, 0.27 mmol, 1 equiv) dropwise over 3 min at 0° C. The resulting mixture was stirred at room temperature for additional overnight. The resulting mixture was filtered, the filter cake was washed with DCM (10 mL) (3×5 mL). The filtrate was concentrated under reduced pressure. The residue was purified by trituration with Et₂O (5 mL). The precipitated solids were collected by filtration and washed with Et₂O (3×5 mL). This resulted in 2-chloro-3,3-dicyclohexyl-2-{3-[(1-{3-[(fluorosulfonyl)oxy]benzoyl}piperidin-4-yl)amino]phenyl}-1,1-dimethyl-2H,3H-3lambda5-benzo[d]1-aza-3lambda5-phospha-2-auracyclopentan-1-ium-3-ylium-2,2-diuide (95 mg, 34.44% yield, 91.2% purity) as a reddish brown solid. LCMS:(ES, m/z): [M]⁺=926.1.

¹H NMR (300 MHz, DMSO-d₆, ppm) δ 8.33 (dd, J=8.6, 4.4 Hz, 1H), 8.04 (dt, J=14.1, 8.3 Hz, 2H), 7.82-7.63 (m, 4H), 7.56 (dt, J=6.2, 2.1 Hz, 1H), 7.04 (t, J=7.9 Hz, 1H), 6.68-6.63 (m, 1H), 6.56 (d, J=8.2 Hz, 1H), 6.47 (d, J=7.8 Hz, 1H), 5.83 (d, J=8.0 Hz, 1H), 5.76 (s, OH), 4.33 (s, 1H), 3.57 (s, 2H), 3.39 (s, 4H), 3.29-3.06 (m, 3H), 2.93 (s, 2H), 2.15-1.82 (m, 3H), 1.66 (d, J=28.4 Hz, 11H), 1.28 (dd, J=35.5, 10.0 Hz, 7H), 1.04 (d, J=12.7 Hz, 2H), 0.68 (s, 2H).

### Example 45. Synthesis of Au-MM-5

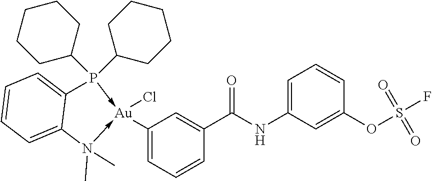

Step 1: To a stirred mixture of tert-butyl N-(3-hydroxyphenyl)carbamate (1.70 g, 7.31 mmol, 1.00 equiv) and 3-(fluorosulfonyl)-1,2-dimethylimidazol-1-ium triflate (3.60 g, 10.96 mmol, 1.5 equiv) in MeCN (5 mL) was added TEA (1.48 g, 14.624 mmol, 2 equiv) dropwise at 0° C. The resulting mixture was stirred at room temperature for additional 30 min. The reaction was monitored by LCMS. The resulting mixture was concentrated under reduced pressure. The residue was purified by silica gel column chromatography, eluted with PE/EA (5:1) to afford tert-butyl N-{3-[(fluorosulfonyl)oxy]phenyl}carbamate (1.2 g, 56.34% yield) as a white solid.

Step 2. To the above mixture was added HCl in dioxane (3 mL, 1 equiv) dropwise over 1 min at 25° C. The resulting mixture was stirred at room temperature for additional 1 h. The resulting mixture was concentrated under reduced pressure. This resulted in 3-aminophenyl sulfurofluoridate (0.8 g, 67.67% yield) as a red crude solid. The crude product was used in the next step directly without further purification.

Step 2: To a stirred mixture of 3-aminophenyl sulfurofluoridate (200 mg, 0.837 mmol, 1.00 equiv, 80%) and 3-iodobenzoic acid (207.57 mg, 0.83 mmol, 1.0 equiv) in DMF (3 mL) were added DIEA (162.25 mg, 1.256 mmol, 1.5 equiv) and HATU (954.66 mg, 2.51 mmol, 3 equiv) in portions at room temperature. The resulting mixture was stirred at room temperature for 2 h. The reaction was monitored by LCMS. The resulting mixture was extracted with EtOAc (3×2 mL). The combined organic layers were washed with water (3×2 mL), dried over anhydrous Na₂SO₄. After filtration, the filtrate was concentrated under reduced pressure. The residue was purified by silica gel column chromatography, eluted with PE/EA (5:1) to afford 3-(3-iodobenzamido)phenyl sulfurofluoridate (100 mg, 28.37% yield) as a white solid.

Step 3: To a stirred mixture of 3-(3-iodobenzamido)phenyl sulfurofluoridate (80 mg, 0.19 mmol, 1 equiv) and 2-[(-[(chloroaurio)dicyclohexyl-lambda5-phosphanyl]-N,N-dimethylaniline (104.64 mg, 0.19 mmol, 1 equiv) in DCM (3 mL) was added Silver Hexafluoroantimonate(V) (65.27 mg, 0.19 mmol, 1 equiv) dropwise at −20° C. The resulting mixture was stirred at room temperature for 2 h. The reaction was monitored by LCMS. The resulting mixture was filtered, the filter cake was washed with DCM (3 mL) (3×1 mL). The filtrate was concentrated under reduced pressure. The residue was purified by trituration with MTBE (2 mL). This resulted in [3-({3-[(fluorosulfonyl)oxy]phenyl}carbamoyl)phenyl]gold (100 mg, 24.95% yield, 94% purity) as a grey solid. LCMS:(ES, m/z): [M]⁺=843.1.

¹H NMR (300 MHz, DMSO-d₆, ppm) δ 10.71 (s, 1H), 8.37 (dd, J=8.5, 4.3 Hz, 1H), 8.12-7.86 (m, 6H), 7.85-7.67 (m, 2H), 7.60 (dt, J=10.6, 8.1 Hz, 2H), 7.36 (dd, J=8.3, 2.6 Hz, 1H), 3.91 (s, 6H), 2.93 (d, J=11.3 Hz, 2H), 1.91 (s, 2H), 1.80-1.52 (m, 10H), 1.40-1.22 (m, 4H), 1.07 (t, J=12.8 Hz, 2H), 0.61 (s, 2H).

### Example 46. Synthesis of Au-MM-6

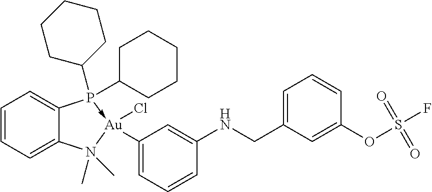

Step 1: To a stirred mixture of 3-hydroxybenzaldehyde (2 g, 16.377 mmol, 1 equiv) and 3-(fluorosulfonyl)-1,2-dimethylimidazol-1-ium triflate (6.45 g, 19.652 mmol, 1.2 equiv) in MeCN (20 mL) was added TEA (2.49 g, 24.566 mmol, 1.5 equiv) in portions at 0° C. The resulting mixture was stirred at 0° C. for 30 min. The resulting mixture was concentrated under reduced pressure. The residue was purified by reversed-phase flash chromatography with the following conditions: column, C18 silica gel; mobile phase, MeCN in Water (0.1% FA), 10% to 95% gradient in 10 min; detector, UV 254 nm. This resulted in 3-formylphenyl sulfurofluoridate (1.8 g, 53.83% yield) as a white solid.

Step 2: To a stirred mixture of 3-formylphenyl sulfurofluoridate (500 mg, 2.449 mmol, 1 equiv) and benzenamine, 3-iodo- (804.57 mg, 3.673 mmol, 1.5 equiv) in DCM (10 mL) were added AcOH (147.06 mg, 2.449 mmol, 1 equiv) and STAB (1038.06 mg, 4.898 mmol, 2 equiv) in portions at 0° C. The resulting mixture was stirred at room temperature for 1 h. The resulting mixture was extracted with CH₂Cl₂ (3×10 mL). The combined organic layers were washed with water (3×10 mL), dried over anhydrous Na₂SO₄. After filtration, the filtrate was concentrated under reduced pressure. The residue was purified by reversed-phase flash chromatography with the following conditions: column, C18 silica gel; mobile phase, MeCN in Water (0.1% FA), 10% to 95% gradient in 10 min; detector, UV 254 nm. This resulted in 3-{[(3-iodophenyl) amino]methyl}phenyl sulfurofluoridate (300 mg, 30.08% yield) as a white solid.

Step 3: To a stirred solution of 3-{[(3-iodophenyl) amino]methyl}phenyl sulfurofluoridate (100 mg, 0.246 mmol, 1 equiv) and (chloroaurio)dicyclohexyl[2-(dimethylamino) phenyl]-lambda5-phosphanyl (135.04 mg, 0.246 mmol, 1 equiv) in DCM (3 mL) were added Silver Hexafluoroantimonate(V) (84.39 mg, 0.246 mmol, 1 equiv) in portions at 0° C. under nitrogen atmosphere. The resulting mixture was stirred at room temperature for additional 2 h. The reaction was monitored by LCMS. The resulting mixture was filtered, the filter cake was washed with DCM (3×3 mL). The filtrate was concentrated under reduced pressure. The residue was purified by trituration with Methyl tert-butyl ether (3 mL). This resulted in [chloro({3-[({3-[(fluorosulfonyl) oxy]phenyl}methyl) amino]phenyl}) aurio]dicyclohexyl[2-(dimethylamino)phenyl]-lambda5-phosphanyl (103.7 mg, 50.87% yield, 94.8% purity) as a yellow solid. LCMS:(ES, m/z): [M]⁺=829.2.

¹H NMR (300 MHz, DMSO-d₆, ppm) δ 8.33 (dd, J=8.4, 4.5 Hz, 1H), 8.12-7.94 (m, 2H), 7.77 (dt, J=8.5, 4.2 Hz, 1H), 7.66-7.39 (m, 4H), 7.02 (t, J=7.9 Hz, 1H), 6.66 (s, 1H), 6.59-6.40 (m, 2H), 4.45 (s, 2H), 3.51-3.26 (m, 6H), 2.91 (s, 2H), 1.89-1.42 (m, 12H), 1.29 (dt, J=29.9, 13.5 Hz, 4H), 1.04 (t, J=12.7 Hz, 2H), 0.63 (s, 2H).

### Example 47. Synthesis of Au-PM-1

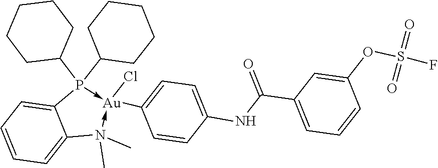

Step 1: To a stirred solution of 3-[(fluorosulfonyl)oxy]benzoic acid (552.88 mg, 2.511 mmol, 1.1 equiv), DIEA (885.15 mg, 6.849 mmol, 3 equiv) and HATU (1302.03 mg, 3.425 mmol, 1.5 equiv) in DCM (12 mL) were added 4-iodo-benzenamine (500 mg, 2.283 mmol, 1.00 equiv) dropwise at 0° C. The resulting mixture was stirred at 25° C. for additional 1 h. The reaction was monitored by LCMS. The resulting mixture was extracted with DCM (3×5 mL). The combined organic layers were dried over anhydrous Na₂SO₄. After filtration, the filtrate was concentrated under reduced pressure. The residue was purified by reversed-phase flash chromatography with the following conditions: column, C18 silica gel; mobile phase, MeCN in Water (0.1% FA), 10% to 60% gradient in 12 min; detector, UV 254 nm. This resulted in 3-[(4-iodophenyl)carbamoyl]phenyl sulfurofluoridate (300 mg, 31.20% yield) as a yellow oil.

Step 2: To a stirred mixture of AgSbF₆ (163.17 mg, 0.475 mmol, 1 equiv) in DCM (8 mL) were added 3-[(4-iodophenyl)carbamoyl]phenyl sulfurofluoridate (200 mg, 0.475 mmol, 1 equiv) and (chloroaurio)dicyclohexyl[2-(dimethylamino)phenyl]-lambda5-phosphanyl (261.11 mg, 0.475 mmol, 1.00 equiv) in portions at −20° C. The resulting mixture was stirred at 25° C. for additional 2 h. The resulting mixture was filtered, the filter cake was washed with MeCN. The filtrate was concentrated under reduced pressure. The residue was purified by trituration with Et₂O (2 mL). This resulted in product (80 mg, 19.96% yield) as a light grey solid. LCMS: (ES, m/z): [M]⁺=843.0.

¹H NMR (300 MHz, DMSO-d₆, ppm) δ 10.54 (s, 1H), 8.35 (dd, J=8.6, 4.3 Hz, 1H), 8.21-7.96 (m, 4H), 7.96-7.71 (m, 5H), 7.43 (d, J=8.5 Hz, 2H), 3.44 (s, 6H), 2.97-2.93 (m, 2H), 1.89 (m, 2H), 1.72-1.63 (m, 10H), 1.47-1.20 (m, 4H), 1.16-0.97 (m, 2H), 0.67 (m, 2H).

### Example 48. Synthesis of Au-PM-4

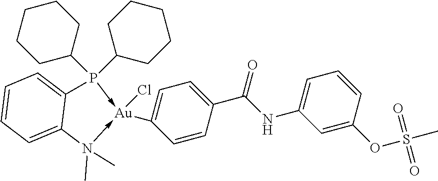

Step 1: To a stirred mixture of tert-butyl N-(3-hydroxyphenyl)carbamate (1.7 g, 7.31 mmol, 1.00 equiv) and 3-(fluorosulfonyl)-1,2-dimethylimidazol-1-ium triflate (3.60 g, 10.968 mmol, 1.5 equiv) in MeCN (5 mL) was added TEA (1.48 g, 14.624 mmol, 2 equiv) dropwise at 0° C. The resulting mixture was stirred at room temperature for additional 30 min. The reaction was monitored by LCMS. The resulting mixture was concentrated under reduced pressure. The residue was purified by silica gel column chromatography, eluted with PE/EA (5:1) to afford tert-butyl N-{3-[(fluorosulfonyl)oxy]phenyl}carbamate (1.2 g, 56.34% yield) as a white solid.

Step 2: To the above mixture was added HCl in dioxane (3 mL, 1 equiv) dropwise over 1 min at 25° C. The resulting mixture was stirred at room temperature for additional 1 h. The resulting mixture was concentrated under reduced pressure. This resulted in 3-aminophenyl sulfurofluoridate (0.8 g, 67.67% yield) as a red crude solid. The crude product was used in the next step directly without further purification.

Step 3: To a stirred mixture of 3-aminophenyl sulfurofluoridate (200 mg, 0.83 mmol, 1.00 equiv, 80%) and 4-iodobenzoic acid (207.57 mg, 0.837 mmol, 1.0 equiv) in DMF were added DIEA (162.25 mg, 1.256 mmol, 1.5 equiv) and HATU (954.66 mg, 2.511 mmol, 3 equiv) in portions at room temperature. The resulting mixture was stirred at room temperature for 2 h. The reaction was monitored by LCMS. The resulting mixture was extracted with EtOAc (3×2 mL). The combined organic layers were washed with water (3×2 mL), dried over anhydrous Na₂SO₄. After filtration, the filtrate was concentrated under reduced pressure. The residue was purified by silica gel column chromatography, eluted with PE/EA (5:1) to afford 3-(4-iodobenzamido)phenyl sulfurofluoridate (150 mg, 28.37% yield, 90% purity) as a white solid.

Step 4: To a stirred solution of 3-(4-iodobenzamido)phenyl sulfurofluoridate (100 mg, 0.19 mmol, 1 equiv) and 2-[(chloroaurio)dicyclohexyl-lambda5-phosphanyl]-N,N-dimethylaniline (104.64 mg, 0.19 mmol, 1 equiv) in DCM (3 mL) was added Silver Hexafluoroantimonate(V) (65.27 mg, 0.190 mmol, 1 equiv) dropwise at −20° C. The resulting mixture was stirred at room temperature for 2 h. The reaction was monitored by LCMS. The resulting mixture was filtered, the filter cake was washed with DCM (3 mL) (3×1 mL). The filtrate was concentrated under reduced pressure. The residue was purified by trituration with MTBE (2 mL). This resulted in [3-({4-[(fluorosulfonyl)oxy]phenyl}carbamoyl)phenyl]gold (150 mg, 36.25% yield, 97.6% purity) as a grey solid. LCMS:(ES, m/z): [M]⁺=843.1.

¹H NMR (300 MHz, DMSO-d₆, ppm) δ 10.70 (s, 1H), 8.37 (dd, J=8.5, 4.3 Hz, 1H), 8.17-7.93 (m, 5H), 7.89 (dd, J=8.5, 1.9 Hz, 1H), 7.80 (td, J=7.5, 2.4 Hz, 1H), 7.71-7.56 (m, 3H), 7.36 (dd, J=8.2, 2.5 Hz, 1H), 3.46 (s, 6H), 2.96 (d, J=11.0 Hz, 2H), 1.91 (s, 2H), 1.81-1.49 (m, 10H), 1.32 (dt, J=25.2, 13.1 Hz, 4H), 1.08 (t, J=13.1 Hz, 2H), 0.64 (s, 2H).

### Example 49. Synthesis of Au-PM-6

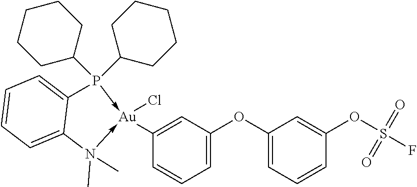

Step 1: A solution of 3-(4,4,5,5-tetramethyl-1,3,2-dioxaborolan-2-yl) phenol (2 g, 9.08 mmol, 1 equiv) in MeCN (3 mL) was treated with 3-(fluorosulfonyl)-1,2-dimethylimidazol-1-ium triflate (4.47 g, 13.63 mmol, 1.5 equiv) at room temperature for 3 min followed by the addition of TEA (1.84 g, 18.17 mmol, 2 equiv) dropwise at 0° C. The reaction was monitored by LCMS. The crude product was purified by reverse phase flash to afford 3-(4,4,5,5-tetramethyl-1,3,2-dioxaborolan-2-yl) phenyl sulfurofluoridate (1 g, 36.42% yield) as a red solid.

Step 2: To a stirred mixture of 3-(4,4,5,5-tetramethyl-1,3,2-dioxaborolan-2-yl) phenyl sulfurofluoridate (1 g, 3.31 mmol, 1 equiv) and 4-iodophenol (0.73 g, 3.31 mmol, 1 equiv) in 1,4-dioxane were added Cu(NO₃)₂ (0.93 g, 4.96 mmol, 1.5 equiv) and TMEDA (1.15 g, 9.93 mmol, 3 equiv) in portions at room temperature. The resulting mixture was stirred at 60° C. for additional 2 h. The reaction was monitored by LCMS. The resulting mixture was concentrated under reduced pressure. The residue/crude product was purified by reverse phase flash to afford 3-(4-iodophenoxy) phenyl sulfurofluoridate as a white solid.

Step 3: To a stirred mixture of 3-(4-iodophenoxy) phenyl sulfurofluoridate (51 mg, 0.12 mmol, 1 equiv) and 2-[(chloroaurio)dicyclohexyl-lambda5-phosphanyl]-N,N-dimethylaniline (71.28 mg, 0.12 mmol, 1 equiv) in DCM (3 mL) were added Silver Hexafluoroantimonate(V) (44.46 mg, 0.12 mmol, 1 equiv) dropwise at 0° C. The resulting mixture was stirred at 0° C. for 2 h under nitrogen atmosphere. The reaction was monitored by LCMS. The resulting mixture was filtered, the filter cake was washed with DCM (3 mL) (3×1 mL). The filtrate was concentrated under reduced pressure. The residue was purified by trituration with Et₂O (3 mL). This resulted in 3-(4-{2-chloro-3,3-dicyclohexyl-1,1-dimethylbenzo[d]1-aza-3-phospha-2-auracyclopentan-2-yl}phenoxy) phenyl sulfurofluoridate (73 mg, 69.04% yield, 92.5% purity) as a white solid. LCMS:(ES, m/z): [M]⁺=816.1.

¹H NMR (300 MHz, DMSO-d₆, ppm) δ 8.36 (dd, J=8.4, 4.4 Hz, 1H), 8.12-7.98 (m, 2H), 7.80 (td, J=7.5, 2.3 Hz, 1H), 7.65 (t, J=8.4 Hz, 1H), 7.52 (d, J=8.7 Hz, 2H), 7.41 (dd, J=8.4, 2.4 Hz, 1H), 7.28-7.19 (m, 3H), 7.15 (dd, J=8.2, 2.3 Hz, 1H), 3.45 (s, 6H), 2.98 (d, J=11.1 Hz, 2H), 1.91 (s, 2H), 1.82-1.47 (m, 10H), 1.44-1.26 (m, 4H), 1.06 (t, J=12.6 Hz, 2H), 0.59 (s, 2H).

### Example 50. Synthesis of Au—PP-4

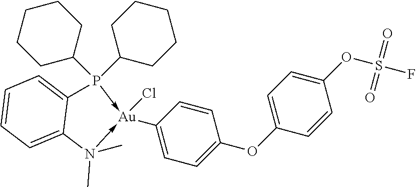

Step 1: To a stirred solution of 4-(4,4,5,5-tetramethyl-1,3,2-dioxaborolan-2-yl) phenol (1 g, 4.544 mmol, 1 equiv) and 3-(fluorosulfonyl)-1,2-dimethylimidazol-1-ium triflate (2.24 g, 6.816 mmol, 1.5 equiv) in MeCN (10 mL) were added TEA (0.92 g, 9.088 mmol, 2 equiv) dropwise at 0° C. under nitrogen atmosphere. The resulting mixture was stirred at room temperature for additional 30 min. The residue was purified by silica gel column chromatography, eluted with PE/THF (5:1) to afford 4-(4,4,5,5-tetramethyl-1,3,2-dioxaborolan-2-yl)phenyl sulfurofluoridate (500 mg, 36.42% yield) as a light yellow oil.

Step 2: To a stirred solution of 4-(4,4,5,5-tetramethyl-1,3,2-dioxaborolan-2-yl) phenylsulfurofluoridate (500 mg, 1.655 mmol, 1 equiv),4-iodophenol (291.29 mg, 1.324 mmol, 0.8 equiv), TMEDA (288.48 mg, 2.482 mmol, 1.5 equiv) and Cu(NO₃)₂ (620.79 mg, 3.310 mmol, 2 equiv) in dioxane (10 mL) at room temperature under Oxygen atmosphere. The resulting mixture was stirred at 60° C. for additional overnight under Oxygen atmosphere. The resulting mixture was diluted with 10 mL H₂O and extracted with EtOAc (10 mL×3). The combined organic layers were washed with brine (10 mL), dried over anhydrous Na₂SO₄. After filtration, the filtrate was concentrated under reduced pressure. The residue was purified by reversed-phase flash chromatography with the following conditions: column, C18 silica gel; mobile phase, MeCN in Water (0.1% TFA), 10% to 50% gradient in 10 min; detector, UV 254 nm. This resulted in 4-(4-iodophenoxy) phenyl sulfurofluoridate (80 mg, 12.26% yield) as a light yellow oil.

Step 3: To a stirred solution of Silver Hexafluoroantimonate(V) (69.74 mg, 0.203 mmol, 1 equiv) in DCM (3 mL) at −10° C. under nitrogen atmosphere. To the above mixture was added 4-(4-iodophenoxy)phenyl sulfurofluoridate (80 mg, 0.203 mmol, 1 equiv) and (chloroaurio)dicyclohexyl[2-(dimethylamino)phenyl]-lambda5-phosphanyl (111.61 mg, 0.203 mmol, 1 equiv) in portions over 2 min at −10° C. The resulting mixture was stirred at room temperature for additional overnight. The resulting mixture was filtered, the filter cake was washed with DCM (1 mL×3). The residue was purified by trituration with Methyl tert-butyl ether (3 mL). This resulted in 2-chloro-3,3-dicyclohexyl-2-(4-{4-[(fluorosulfonyl)oxy]phenoxy}phenyl)-1,1-dimethyl-2H,3H-3lambda5-benzo[d]1-aza-3lambda5-phospha-2-auracyclopentan-1-ium-3-ylium-2,2-diuide (47.9 mg, 28.88% yield, 88% purity) as a yellow oil. LCMS−:(ES, m/z): [M]⁺=816.2.

¹H NMR (300 MHz, DMSO-d₆, ppm) δ 8.35 (dd, J=8.4, 4.4 Hz, 1H), 8.14-7.98 (m, 2H), 7.79 (td, J=7.4, 2.4 Hz, 1H), 7.72-7.63 (m, 2H), 7.49 (d, J=8.5 Hz, 2H), 7.24-7.13 (m, 4H), 3.44 (s, 6H), 2.97 (q, J=11.2 Hz, 2H), 1.90 (s, 2H), 1.80-1.46 (m, 10H), 1.33 (dt, J=26.9, 13.5 Hz, 4H), 1.02 (q, J=9.8, 7.2 Hz, 2H), 0.65-0.45 (m, 2H).

### Example 51. Synthesis of Au—PP-5

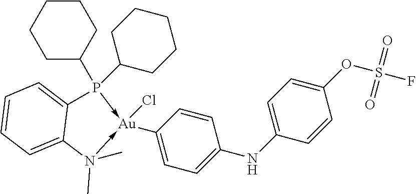

Step 1: To a stirred solution of P-anisidine (2 g, 16.240 mmol, 1 equiv),1,4-diiodobenzene (8.04 g, 24.360 mmol, 1.5 equiv), Pd(dppf)Cl₂ (5.94 g, 8.120 mmol, 0.5 equiv) and t-BuONa (3.12 g, 32.480 mmol, 2 equiv) in toluene (20 mL) at room temperature under nitrogen atmosphere. The resulting mixture was stirred at 70° C. for additional 2 h. The mixture was allowed to cool down to room temperature. The resulting mixture was diluted with 20 mL H₂O and extracted with EtOAc (20 mL×3). The combined organic layers were washed with brine (20 mL), dried over anhydrous Na₂SO₄. After filtration, the filtrate was concentrated under reduced pressure. The residue was purified by silica gel column chromatography, eluted with PE/THF (4:1) to afford 4-iodo-N-(4-methoxyphenyl)aniline (1.8 g, 34.09% yield) as a light yellow solid.

Step 2: To a stirred solution of 4-iodo-N-(4-methoxyphenyl) aniline (900 mg, 2.768 mmol, 1 equiv) in DCM (3 mL) was added Boron tribromide 1M solution in methylene chloride (5.54 mL, 5.540 mmol, 2.00 equiv) dropwise at 0° C. under nitrogen atmosphere. The resulting mixture was stirred at room temperature for additional 1 h. The reaction was quenched by the addition of ice water (5 mL) at 0° C. The resulting mixture was diluted with 5 mL H₂O and extracted with DCM (10 mL×3). The combined organic layers were washed with brine (10 mL), dried over anhydrous Na₂SO₄. After filtration, the filtrate was concentrated under reduced pressure. This resulted in 4-[(4-iodophenyl) amino]phenol (600 mg, 69.67% yield) as a light brown solid. The crude product was used in the next step directly without further purification.

Step 3: To a stirred solution of 4-[(4-iodophenyl) amino]phenol (300 mg, 0.964 mmol, 1 equiv) and 3-(fluorosulfonyl)-1,2-dimethylimidazol-1-ium triflate (474.77 mg, 1.446 mmol, 1.5 equiv) in MeCN (5 mL) were added TEA (195.15 mg, 1.928 mmol, 2 equiv) dropwise at 0° C. under nitrogen atmosphere. The resulting mixture was stirred at room temperature for additional 30 min. The resulting mixture was concentrated under reduced pressure. The residue was purified by reversed-phase flash chromatography with the following conditions: column, C18 silica gel; mobile phase, MeCN in Water (0.1% FA), 10% to 90% gradient in 10 min; detector, UV 254 nm. This resulted in 4-[(4-iodophenyl) amino]phenyl sulfurofluoridate (150 mg, 39.57% yield) as a light yellow oil.

Step 4: To a stirred solutione of Silver Hexafluoroantimonate(V) (131.10 mg, 0.382 mmol, 1 equiv) in DCM (2 mL) at −10° C. under nitrogen atmosphere. To the above mixture was added 4-[(4-iodophenyl) amino]phenyl sulfurofluoridate (150 mg, 0.382 mmol, 1 equiv) and (chloroaurio)dicyclohexyl[2-(dimethylamino) phenyl]-lambda5-phosphanyl (209.78 mg, 0.382 mmol, 1 equiv) in portions over 2 min at −10° C. The resulting mixture was stirred at room temperature for additional overnight. The resulting mixture was filtered, the filter cake was washed with DCM (1 mL×3). The filtrate was concentrated under reduced pressure. The residue was purified by trituration with Methyl tert-butyl ether (3 mL). This resulted in {[2-({chloro[4-({4-[(fluorosulfonyl) oxy]phenyl}amino) phenyl]aurio}dicyclohexyl-lambda5-phosphanyl) phenyl]dimethylammonio}methanidylidene (100 mg, 31.65% yield, 91.6% purity) as a yellow solid. LCMS:(ES, m/z): [M]⁺=815.2.

¹H NMR (300 MHz, DMSO-d₆, ppm) δ 8.64 (s, 1H), 8.34 (dd, J=8.5, 4.4 Hz, 1H), 8.15-7.97 (m, 2H), 7.78 (t, J=6.9 Hz, 1H), 7.45 (d, J=8.9 Hz, 2H), 7.28 (d, J=8.5 Hz, 2H), 7.17 (dd, J=8.7, 5.7 Hz, 4H), 2.95 (d, J=11.7 Hz, 2H), 1.87 (s, 2H), 1.80-1.45 (m, 10H), 1.47-1.24 (m, 4H), 1.11-1.01 (m, 2H), 0.70-0.55 (m, 2H).

### Example 52. Synthesis of 2-((2,5-dioxo-2,5-dihydro-1H-pyrrol-1-yl)methyl)-6-fluorophenyl sulfurofluoridate (MFS-3-3)

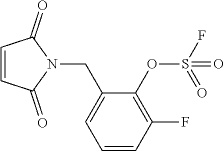 [SMILES: O=C1C=CC(=O)N1Cc1cccc(F)c1OS(=O)(=O)F]

Step 1: To a stirred mixture of 3-fluoro-2-hydroxybenzonitrile (1 g, 7.293 mmol, 1 equiv) in Et₂O (20 mL) was added LiAlH₄ (7.29 mL, 14.586 mmol, 2 equiv) dropwise at 0° C. The resulting mixture was stirred at room temperature for 4 h. To the above mixture was added disodium decahydrate sulfate in portions at 0° C. The resulting mixture was stirred at 0° C. for additional 10 min. The resulting mixture was filtered, the filter cake was washed with Et₂O (3×10 mL). The filtrate was concentrated under reduced pressure. The residue was purified by reversed-phase flash chromatography with the following conditions: column, C18 silica gel; mobile phase, MeCN in Water (0.1% FA), 10% to 85% gradient in 10 min; detector, UV 254 nm. This resulted in 2-(aminomethyl)-6-fluorophenol (800 mg, 77.71% yield) as a white solid.

Step 2: A mixture of 2-(aminomethyl)-6-fluorophenol (500 mg, 3.542 mmol, 1 equiv), maleic anhydride (416.84 mg, 4.250 mmol, 1.2 equiv) and 4A Molecular Sievesin in AcOH (10 mL) was stirred at 120° C. for 12 h. The resulting mixture was concentrated under reduced pressure. The residue was purified by reversed-phase flash chromatography with the following conditions: column, C18 silica gel; mobile phase, MeCN in Water (0.1% FA), 10% to 95% gradient in 10 min; detector, UV 254 nm. This resulted in 1-[(3-fluoro-2-hydroxyphenyl) methyl]pyrrole-2,5-dione (300 mg, 38.29% yield) as a brown oil.

Step 3: To a stirred mixture of 1-[(3-fluoro-2-hydroxyphenyl) methyl]pyrrole-2,5-dione (200 mg, 0.904 mmol, 1 equiv) and 3-(fluorosulfonyl)-1,2-dimethylimidazol-1-ium triflate (356.17 mg, 1.085 mmol, 1.2 equiv) in DCM (3 mL) was added TEA (137.25 mg, 1.356 mmol, 1.5 equiv) dropwise at 0° C. The resulting mixture was stirred at 0° C. for 30 min. The residue was purified by silica gel column chromatography, eluted with PE/EA (5:1) to afford 2-[(2,5-dioxopyrrol-1-yl) methyl]-6-fluorophenyl sulfurofluoridate (110 mg, 40.12% yield, 99.4% purity) as a white solid. GCMS:(ES, m/z): [M]⁺=303.0.

¹H NMR (300 MHz, DMSO-d₆, ppm) δ 7.64-7.48 (m, 2H), 7.34-7.25 (m, 1H), 7.13 (s, 2H), 4.74 (s, 2H).

### Example 53. Synthesis of 2-(2-(2,5-dioxo-2,5-dihydro-1H-pyrrol-1-yl)ethyl)phenyl sulfurofluoridate (MFS-4-2)

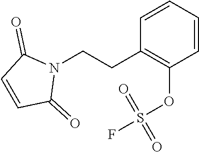 [SMILES: O=C1C=CC(=O)N1CCc1ccccc1OS(=O)(=O)F]

Step 1: A solution of 2-hydroxyphenethylamine (500 mg, 3.64 mmol, 1 equiv) and 2,5-dihydrofuran-2,5-dione (1072.19 mg, 10.93 mmol, 3 equiv) in acetic acid (10 mL) was stirred at 100° C. for overnight under nitrogen atmosphere. The mixture was allowed to cool down to room temperature. The resulting mixture was concentrated under reduced pressure. The residue was purified by silica gel column chromatography, eluted with PE/EA (5:1) to afford 1-[2-(2-hydroxyphenyl) ethyl]pyrrole-2,5-dione (450 mg, 56.84% yield, 95% purity) as a yellow oil.

Step 2: A solution of 1-[2-(2-hydroxyphenyl) ethyl]pyrrole-2,5-dione (450 mg, 2.07 mmol, 1 equiv) in MeCN (10 mL) was treated with 3-(fluorosulfonyl)-1,2-dimethylimidazol-1-ium triflate (1360.00 mg, 4.14 mmol, 2 equiv) at 0° C. for 3 min under nitrogen atmosphere followed by the addition of Et₃N (838.52 mg, 8.28 mmol, 4 equiv) dropwise at 0° C. The resulting mixture was stirred at 0° C. for additional 1 h. The resulting mixture was concentrated under reduced pressure. The residue was purified by reversed-phase flash chromatography with the following conditions: column, C18 silica gel; mobile phase, MeCN in Water (0.1% NH₃·H₂O+10 mmol/L NH₄HCO₃), 10% to 50% gradient in 10 min; detector, UV 254 nm. This resulted in 2-[2-(2,5-dioxopyrrol-1-yl) ethyl]phenyl sulfurofluoridate (151.7 mg, 24.47% yield, 100% purity) as a yellow solid. GCMS:(GS, m/z): [M]⁺=299.0.

¹H NMR (300 MHz, DMSO-d₆, ppm) δ 7.76-7.32 (m, 4H), 6.99 (s, 2H), 3.68 (t, J=6.8 Hz, 2H), 2.95 (t, J=6.8 Hz, 2H).

### Example 54. Synthesis of 5-((2,5-dioxo-2,5-dihydro-1H-pyrrol-1-yl)methyl)-2-fluorophenyl sulfurofluoridate (MFS-4-4)

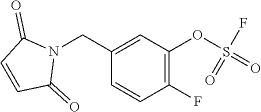 [SMILES: O=C1C=CC(=O)N1Cc1ccc(F)c(OS(=O)(=O)F)c1]

Step 1: A solution of 4-fluoro-hydroxybenzonitrile (1.5 g, 10.94 mmol, 1 equiv) in Diethyl ether (20 mL) at 0° C. for 3 min under nitrogen atmosphere followed by the addition of Lithium aluminum hydriden (1.0 M in THF) (0.62 g, 16.41 mmol, 1.5 equiv) dropwise at 0° C. The resulting mixture was stirred at 0° C. for 2 h under nitrogen atmosphere. The reaction was quenched by the addition of water (20 mL) at 0° C. The mixture was acidified to pH 3 with conc. HCl. The resulting mixture was extracted with EtOAc (2×30 mL). The combined organic layers were washed with brine (1×30 mL), dried over anhydrous Na₂SO₄. After filtration, the filtrate was concentrated under reduced pressure. The residue was purified by silica gel column chromatography, eluted with PE/EA (5:1) to afford 5-(aminomethyl)-2-fluorophenol (1 g, 64.76% yield, 95% purity) as a yellow oil.

Step 2: A solution of 5-(aminomethyl)-2-fluorophenol (1 g, 7.08 mmol, 1 equiv) and maleic anhydride (2.08 g, 21.25 mmol, 3 equiv) in HOAc (10 mL) was stirred at 120° C. for overnight under nitrogen atmosphere. The mixture was allowed to cool down to room temperature. The resulting mixture was concentrated under reduced pressure. The residue was purified by reversed-phase flash chromatography with the following conditions: column, C18 silica gel; mobile phase, MeCN in Water (0.1% FA), 10% to 50% gradient in 10 min; detector, UV 254 nm. This resulted in 1-[(4-fluoro-3-hydroxyphenyl) methyl]pyrrole-2,5-dione (190 mg, 12.12% yield, 95% purity) as a yellow oil.

Step 3: A solution of 1-[(4-fluoro-3-hydroxyphenyl) methyl]pyrrole-2,5-dione (98.48 mg, 0.44 mmol, 1 equiv) in MeCN (10 mL) was treated with 3-(fluorosulfonyl)-1,2-dimethylimidazol-1-ium triflate (190 mg, 0.57 mmol, 1.3 equiv) at 0° C. for 3 min under nitrogen atmosphere followed by the addition of Et₃N (135.17 mg, 1.336 mmol, 3 equiv) dropwise at 0° C. The resulting mixture was stirred at 0° C. for additional 1 h. The resulting mixture was concentrated under reduced pressure. The residue was purified by reversed-phase flash chromatography with the following conditions: column, C18 silica gel; mobile phase, MeCN in Water (0.1% FA), 10% to 50% gradient in 10 min; detector, UV 254 nm. This resulted in 5-[(2,5-dioxopyrrol-1-yl) methyl]-2-fluorophenyl sulfurofluoridate (102.9 mg, 76.21% yield, 96.7% purity) as a yellow solid. GCMS:(ES, m/z): [M]⁺=303.1.

¹H NMR (300 MHz, DMSO-d₆, ppm) δ 7.77-7.68 (m, 1H), 7.59 (dd, J=10.2, 8.6 Hz, 1H), 7.44 (ddd, J=8.6, 4.6, 2.2 Hz, 1H), 7.10 (s, 2H), 4.66 (s, 2H).

### Example 55. Synthesis of (R)-3-(3-(2,5-dioxo-2,5-dihydro-1H-pyrrol-1-yl)pyrrolidin-1-yl)phenyl sulfurofluoridate (MFS-6-2)

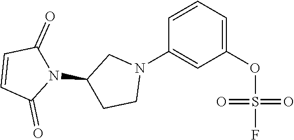 [SMILES: O=C1C=CC(=O)N1[C@@H]1CCN(c2cccc(OS(=O)(=O)F)c2)C1]

Step 1: A solution of tert-butyl N-[(3R)-pyrrolidin-3-yl]carbamate (3 g, 16.10 mmol, 1 equiv), 1-(benzyloxy)-3-bromobenzene (5.09 g, 19.32 mmol, 1.2 equiv), XPhos (1.54 g, 3.22 mmol, 0.2 equiv), t-BuONa (4.64 g, 48.32 mmol, 3 equiv) and Pd₂(dba)₃ (1.47 g, 1.61 mmol, 0.1 equiv) in toluene (30 mL) was stirred at 100° C. for overnight under nitrogen atmosphere. The mixture was allowed to cool down to room temperature. The resulting mixture was filtered, the filter cake was washed with toluene (3×10 mL). The filtrate was concentrated under reduced pressure. The residue was purified by reversed-phase flash chromatography with the following conditions: column, C18 silica gel; mobile phase, MeCN in Water (0.1% FA), 10% to 50% gradient in 10 min; detector, UV 254 nm. This resulted in tert-butyl N-[(3R)-1-[3-(benzyloxy) phenyl]pyrrolidin-3-yl]carbamate (2.5 g, 42.12% yield, 95% purity) as a yellow oil.

Step 2: A solution of tert-butyl N-[(3R)-1-[3-(benzyloxy) phenyl]pyrrolidin-3-yl]carbamate (2.5 g, 6.78 mmol, 1 equiv) and Pd/C (0.36 g, 3.39 mmol, 0.5 equiv) in MeOH (25 mL) was stirred at room temperature for overnight under nitrogen atmosphere. The resulting mixture was filtered, the filter cake was washed with MeOH (3×10 mL). The filtrate was concentrated under reduced pressure. This resulted in tert-butyl N-[(3R)-1-(3-hydroxyphenyl)

Step 3: A solution of tert-butyl N-[(3R)-1-(3-hydroxyphenyl) pyrrolidin-3-yl]carbamate (1.7 g, 6.10 mmol, 1 equiv) in MeCN (20 mL) was treated with 1-(fluorosulfonyl)-2,3-dimethyl-1H-imidazol-3-ium trifluoromethanesulfonate (2.61 g, 7.93 mmol, 1.3 equiv) at 0° C. for 2 min under nitrogen atmosphere followed by the addition of TEA (1.85 g, 18.32 mmol, 3 equiv) dropwise at 0° C. The resulting mixture was stirred at 0° C. for 1 h under nitrogen atmosphere. The resulting mixture was concentrated under reduced pressure. The residue was purified by reversed-phase flash chromatography with the following conditions: column, C18 silica gel; mobile phase, MeCN in Water (0.1% FA), 10% to 50% gradient in 10 min; detector, UV 254 nm. This resulted in tert-butyl N-[(3R)-1-{3-[(fluorosulfonyl) oxy]phenyl}pyrrolidin-3-yl]carbamate (1.1 g, 49.98% yield, 95% purity) as a yellow oil.

Step 4: A solution of tert-butyl N-[(3R)-1-{3-[(fluorosulfonyl)oxy]phenyl}pyrrolidin-3-yl]carbamate (1.1 g, 3.05 mmol, 1 equiv) and HCl in 1,4-dioxane (4.0 M) (5 mL) in DCM (5 mL) was stirred at room temperature for 2 h under nitrogen atmosphere. The resulting mixture was concentrated under reduced pressure. This resulted in 3-[(3R)-3-aminopyrrolidin-1-yl]phenyl sulfurofluoridate (1.1 g, Crude Product) as a white solid.

Step 5: A solution of 3-[(3R)-3-aminopyrrolidin-1-yl]phenyl sulfurofluoridate (1.1 g, 4.22 mmol, 1 equiv), 2,5-dihydrofuran-2,5-dione (0.83 g, 8.45 mmol, 2 equiv) and TEA (1.28 g, 12.67 mmol, 3 equiv) in MeCN (15 mL) was stirred at 80° C. for overnight under nitrogen atmosphere. The mixture was allowed to cool down to room temperature. The resulting mixture was concentrated under reduced pressure. The residue was purified by reversed-phase flash chromatography with the following conditions: column, C18 silica gel; mobile phase, MeCN in Water (0.1% FA), 10% to 50% gradient in 10 min; detector, UV 254 nm. This resulted in (2Z)-3-{[(3R)-1-{3-[(fluorosulfonyl)oxy]phenyl}pyrrolidin-3-yl]carbamoyl}prop-2-enoic acid (600 mg, 39.62% yield, 95% purity) as a yellow oil.

Step 6: A solution of (2Z)-3-{[(3R)-1-{3-[(fluorosulfonyl) oxy]phenyl}pyrrolidin-3-yl]carbamoyl}prop-2-enoic acid (350 mg, 0.97 mmol, 1 equiv) and maleic anhydride (287.32 mg, 2.93 mmol, 3 equiv) in HOAc (5 mL) was stirred at 100° C. for overnight under nitrogen atmosphere. The mixture was allowed to cool down to room temperature. The residue was purified by reversed-phase flash chromatography with the following conditions: column, C18 silica gel; mobile phase, MeCN in Water (0.1% FA), 10% to 50% gradient in 10 min; detector, UV 254 nm. This resulted in 3-[(3R)-3-(2,5-dioxopyrrol-1-yl) pyrrolidin-1-yl]phenyl sulfurofluoridate (74.3 mg, 22.35% yield, 95.5% purity) as a red oil. LCMS:(ES, m/z): [M+H]⁺=341.1.

¹H NMR (400 MHz, DMSO-d₆, ppm) δ 7.34 (t, J=8.3 Hz, 1H), 7.03 (s, 2H), 6.75-6.70 (m, 1H), 6.65-6.58 (m, 2H), 4.77 (d, J=7.8 Hz, 1H), 3.58-3.44 (m, 3H), 3.36 (d, J=7.9 Hz, 1H), 2.47-2.36 (m, 1H), 2.29-2.21 (m, 1H).

### Example 56. Synthesis of (S)-3-(3-(2,5-dioxo-2,5-dihydro-1H-pyrrol-1-yl)pyrrolidin-1-yl)phenyl sulfurofluoridate (MFS-6-3)

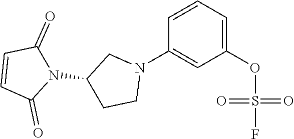 [SMILES: O=C1C=CC(=O)N1[C@H]1CCN(c2cccc(OS(=O)(=O)F)c2)C1]

Step 1: A solution of tert-butyl N-[(3S)-pyrrolidin-3-yl]carbamate (3 g, 16.10 mmol, 1 equiv), 1-(benzyloxy)-3-bromobenzene (5.09 g, 19.32 mmol, 1.2 equiv), Pd₂(dba)₃ (1.47 g, 1.61 mmol, 0.1 equiv), XPhos (1.54 g, 3.22 mmol, 0.2 equiv) and t-BuONa (4.64 g, 48.32 mmol, 3 equiv) in toluene (30 mL) was stirred at 100° C. for overnight under nitrogen atmosphere. The mixture was allowed to cool down to room temperature. The resulting mixture was filtered, the filter cake was washed with MeCN (3×10 mL). The filtrate was concentrated under reduced pressure. The residue was purified by reversed-phase flash chromatography with the following conditions: column, C18 silica gel; mobile phase, MeCN in Water (0.1% FA), 10% to 50% gradient in 10 min; detector, UV 254 nm. This resulted in tert-butyl N-[(3S)-1-[3-(benzyloxy)phenyl]pyrrolidin-3-yl]carbamate (3.3 g, 55.60% yield, 95% purity) as a yellow oil.

Step 2: A solution of tert-butyl N-[(3S)-1-[3-(benzyloxy) phenyl]pyrrolidin-3-yl]carbamate (3.3 g, 8.956=mmol, 1 equiv) and Pd/C (0.48 g, 4.478=mmol, 0.5 equiv) in MeOH (35 mL) was stirred at room temperature for overnight under nitrogen atmosphere. The resulting mixture was filtered, the filter cake was washed with MeOH (3×5 mL). The filtrate was concentrated under reduced pressure. This resulted in tert-butyl N-[(3S)-1-(3-hydroxyphenyl) Step 3: A solution of tert-butyl N-[(3S)-1-(3-hydroxyphenyl) pyrrolidin-3-yl]carbamate (2.4 g, 8.62 mmol, 1 equiv) in MeCN (25 mL) was treated with 3-(fluorosulfonyl)-1,2-dimethylimidazol-1-ium triflate (3.68 g, 11.20 mmol, 1.3 equiv) at 0° C. for 3 min under nitrogen atmosphere followed by the addition of TEA (2.62 g, 25.86 mmol, 3 equiv) dropwise at 0° C. The resulting mixture was stirred at 0° C. for 1 h under nitrogen atmosphere. The resulting mixture was concentrated under reduced pressure. The residue was purified by reversed-phase flash chromatography with the following conditions: column, C18 silica gel; mobile phase, MeCN in Water (0.1% FA), 10% to 50% gradient in 10 min; detector, UV 254 nm. This resulted in tert-butyl N-[(3S)-1-{3-[(fluorosulfonyl) oxy]phenyl}pyrrolidin-3-yl]carbamate (1.4 g, 45.05% yield, 95% purity) as a yellow oil.

Step 4: A solution of tert-butyl N-[(3S)-1-{3-[(fluorosulfonyl) oxy]phenyl}pyrrolidin-3-yl]carbamate (1.4 g, 3.88 mmol, 1 equiv) and HCl in 1,4-dioxane (4.0 M) (10 mL) in DCM (10 mL) was stirred at room temperature for 2 h under nitrogen atmosphere. The resulting mixture was concentrated under reduced pressure. This resulted in 3-[(3S)-3-aminopyrrolidin-1-yl]phenyl sulfurofluoridate (1.4 g, Crude Product) as a white solid.

Step 5: A solution of (2Z)-3-{[(3S)-1-{3-[(fluorosulfonyl) oxy]phenyl}pyrrolidin-3-yl]carbamoyl}prop-2-enoic acid (1 g, 2.79 mmol, 1 equiv) and maleic anhydride (0.82 g, 8.37 mmol, 3 equiv) in HOAc (10 mL) was stirred at 120° C. for overnight under nitrogen atmosphere. The mixture was allowed to cool down to room temperature. The resulting mixture was concentrated under reduced pressure. The residue was purified by reversed-phase flash chromatography with the following conditions: column, C18 silica gel; mobile phase, MeCN in Water (0.1% TFA), 10% to 50% gradient in 10 min; detector, UV 254 nm. This resulted in 3-[(3S)-3-(2,5-dioxopyrrol-1-yl) pyrrolidin-1-yl]phenyl sulfurofluoridate (116.8 mg, 12.30% yield, 99.5% purity) as a light yellow oil. LCMS:(ES, m/z): [M+H]⁺=341.1.

¹H NMR (300 MHz, DMSO-d₆, ppm) δ 7.40-7.28 (m, 1H), 7.04 (s, 2H), 6.73 (d, J=8.1 Hz, 1H), 6.68-6.59 (m, 2H), 4.79 (p, J=7.9 Hz, 1H), 3.66-3.42 (m, 3H), 3.37 (d, J=7.6 Hz, 1H), 2.50-2.35 (m, 1H), 2.33-2.16 (m, 1H).

### Example 57. Synthesis of 4-(3-(2,5-dioxo-2,5-dihydro-1H-pyrrol-1-yl)azetidin-1-yl)phenyl sulfurofluoridate (MFS-7-1)

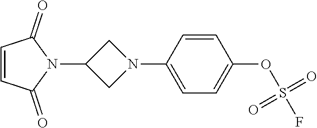 [SMILES: O=C1C=CC(=O)N1C1CN(c2ccc(OS(=O)(=O)F)cc2)C1]

Step 1: To a stirred solution of tert-butyl N-(azetidin-3-yl)carbamate (900 mg, 5.226 mmol, 1 equiv),1-(benzyloxy)-4-bromobenzene (2062.56 mg, 7.839 mmol, 1.5 equiv), Pd₂(dba)₃ (478.53 mg, 0.523 mmol, 0.1 equiv), XPhos (498.24 mg, 1.045 mmol, 0.2 equiv) and t-BuONa (1506.63 mg, 15.678 mmol, 3 equiv) in toluene (15 mL) at room temperature under nitrogen atmosphere. The resulting mixture was stirred at 100° C. for additional overnight. The mixture was allowed to cool down to room temperature. The resulting mixture was diluted with 15 mL H₂O and extracted with EtOAc (15 mL×3). The combined organic layers were washed with brine (15 mL), dried over anhydrous Na₂SO₄. After filtration, the filtrate was concentrated under reduced pressure. The residue was purified by silica gel column chromatography, eluted with PE/THF (3:1) to afford tert-butyl N-{1-[4-(benzyloxy)phenyl]azetidin-3-yl}carbamate (800 mg, 43.19% yield) as a yellow solid.

Step 2: To a stirred solution of tert-butyl N-{1-[4-(benzyloxy) phenyl]azetidin-3-yl}carbamate (800 mg, 2.257 mmol, 1 equiv) and palladium (100 mg, 0.940 mmol, 0.42 equiv) in MeOH (10 mL) at room temperature under nitrogen atmosphere. The resulting mixture was stirred at room temperature for additional 2 h under hydrogen atmosphere. The resulting mixture was filtered, the filter cake was washed with MeOH (5 mL×3). This resulted in tert-butyl N-[1-(4-hydroxyphenyl) azetidin-3-yl]carbamate (500 mg, 83.81% yield) as a light yellow oil. The crude product was used in the next step directly without further purification.

Step 3: To a stirred solution of tert-butyl N-[1-(4-hydroxyphenyl) azetidin-3-yl]carbamate (500 mg, 1.892 mmol, 1 equiv) in DCM (3 mL) was added TFA (3 mL) dropwise at room temperature under nitrogen atmosphere. The resulting mixture was stirred at room temperature for additional 2 h. The precipitated solids were collected by filtration and washed with diethyl ether (2 mL×3). This resulted in 4-(3-aminoazetidin-1-yl) phenol (300 mg, 96.58% yield) as a light yellow solid. The crude product was used in the next step directly without further purification.

Step 4: To a stirred solution of 4-(3-aminoazetidin-1-yl) phenol (300 mg, 1.827 mmol, 1 equiv) and maleic anhydride (268.72 mg, 2.740 mmol, 1.5 equiv) in MeCN (5 mL) was added TEA (369.75 mg, 3.654 mmol, 2 equiv) dropwise at 0° C. under nitrogen atmosphere. The resulting mixture was stirred at room temperature for additional 1 h. The residue was purified by reversed-phase flash chromatography with the following conditions: column, C18 silica gel; mobile phase, MeCN in Water (0.1% FA), 10% to 50% gradient in 10 min; detector, UV 254 nm. This resulted in (2Z)-3-{[1-(4-hydroxyphenyl) azetidin-3-yl]carbamoyl}prop-2-enoic acid (300 mg, 62.61% yield) as a light yellow oil.

Step 5: To a stirred solution of (2Z)-3-{[1-(4-hydroxyphenyl)azetidin-3-yl]carbamoyl}prop-2-enoic acid (200 mg, 0.763 mmol, 1 equiv), HOSu (351.06 mg, 3.052 mmol, 4 equiv) in DMF (5 mL) was added TFAA (640.67 mg, 3.052 mmol, 4 equiv) dropwise at 0° C. under nitrogen atmosphere. The mixture was stirred for 15 min. 2,4,6-trimethylpyridine (184.83 mg, 1.526 mmol, 2 equiv) was added and the mixture was allowed to warm to RT and stirred for overnight. The reaction was quenched by the addition of ice water (3 mL) at 0° C. The resulting mixture was diluted with 3 mL H₂O and extracted with EtOAc (5 mL×3). The combined organic layers were washed with brine (5 mL), dried over anhydrous Na₂SO₄. After filtration, the filtrate was concentrated under reduced pressure. The residue was purified by reversed-phase flash chromatography with the following conditions: column, C18 silica gel; mobile phase, MeCN in Water (0.1% TFA), 10% to 50% gradient in 10 min; detector, UV 254 nm. This resulted in 1-[1-(4-hydroxyphenyl) azetidin-3-yl]pyrrole-2,5-dione (150 mg, 80.53% yield) as a light yellow oil.

Step 6: To a stirred solution of 1-[1-(4-hydroxyphenyl) azetidin-3-yl]pyrrole-2,5-dione (150 mg, 0.614 mmol, 1 equiv) and 3-(fluorosulfonyl)-1,2-dimethylimidazol-1-ium triflate (403.17 mg, 1.228 mmol, 1.5 equiv) in MeCN (3 mL) was added TEA (165.72 mg, 1.638 mmol, 2 equiv) dropwise at 0° C. under nitrogen atmosphere. The resulting mixture was stirred at room temperature for additional 30 min. The resulting mixture was concentrated under reduced pressure. The residue was purified by reversed-phase flash chromatography with the following conditions: column, C18 silica gel; mobile phase, MeCN in Water (0.1% FA), 10% to 90% gradient in 10 min; detector, UV 254 nm. This resulted in 4-[3-(2,5-dioxopyrrol-1-yl) azetidin-1-yl]phenyl sulfurofluoridate (70 mg, 34.93% yield, 99.0% purity) as a light yellow oil. LCMS:(ES, m/z): [M+H]⁺=327.1.

¹H NMR (300 MHz, DMSO-d₆, ppm) δ 7.37 (d, J=8.5 Hz, 2H), 7.03 (d, J=1.5 Hz, 2H), 6.58-6.51 (m, 2H), 4.94 (p, J=7.4 Hz, 1H), 4.20 (dd, J=7.4, 1.5 Hz, 4H).

### Example 58. Synthesis of (R)-4-(3-(2,5-dioxo-2,5-dihydro-1H-pyrrol-1-yl)pyrrolidin-1-yl)phenyl sulfurofluoridate (MFS-7-2)

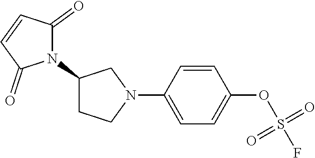 [SMILES: O=C1C=CC(=O)N1[C@@H]1CCN(c2ccc(OS(=O)(=O)F)cc2)C1]

Step 1: To a stirred mixture of tert-butyl N-[(3R)-pyrrolidin-3-yl]carbamate (2 g, 10.738 mmol, 1 equiv), t-BuONa (3.10 g, 32.214 mmol, 3 equiv) and 1-(benzyloxy)-4-bromobenzene (3.39 g, 12.886 mmol, 1.2 equiv) in toluene (40 mL) were added Pd₂(dba)₃ (1.97 g, 2.148 mmol, 0.2 equiv) and XPhos (1.02 g, 2.148 mmol, 0.2 equiv) in portions at room temperature under nitrogen atmosphere. The resulting mixture was stirred at 120° C. for 4 h under nitrogen atmosphere. The resulting mixture was extracted with EtOAc (3×40 mL). The combined organic layers were washed with saline water (3×20 mL), dried over anhydrous Na₂SO₄. After filtration, the filtrate was concentrated under reduced pressure. The residue was purified by reversed-phase flash chromatography with the following conditions: column, C18 silica gel; mobile phase, MeCN in Water (0.1% FA), 10% to 95% gradient in 10 min; detector, UV 254 nm. This resulted in tert-butyl N-[(3R)-1-[4-(benzyloxy)phenyl]pyrrolidin-3-yl]carbamate (2.2 g, 55.60% yield) as a brown solid.

Step 2: To a solution of tert-butyl N-[(3R)-1-[4-(benzyloxy)phenyl]pyrrolidin-3-yl]carbamate (2.2 g, 5.971 mmol, 1 equiv) in 20 mL MeOH was added Pd/C (10%, 0.22 g) under nitrogen atmosphere in a 100 mL round-bottom flask. The mixture was hydrogenated at room temperature for 2 h under hydrogen atmosphere using a hydrogen balloon, filtered through a Celite pad and concentrated under reduced pressure to afford tert-butyl N-[(3R)-1-(4-hydroxyphenyl)pyrrolidin-3-yl]carbamate (1.5 g, 90.26% yield) as a light brown solid. The crude resulting mixture was used in the next step directly without further purification.

Step 3: A solution of tert-butyl N-[(3R)-1-(4-hydroxyphenyl)pyrrolidin-3-yl]carbamate (1.5 g, 5.389 mmol, 1 equiv) in DCM (12 mL) and HCl in 1,4-dioxane (4.0 M) (4 mL) was stirred at room temperature for 2 h. The resulting mixture was concentrated under reduced pressure to afford 4-[(3R)-3-aminopyrrolidin-1-yl]phenol (900 mg, 93.70% yield) as a white solid. The crude resulting mixture was used in the next step directly without further purification.

Step 4: To a stirred mixture of 4-[(3R)-3-aminopyrrolidin-1-yl]phenol (850 mg, 4.769 mmol, 1 equiv) and maleic anhydride (561.16 mg, 5.723 mmol, 1.2 equiv) in ACN (10 mL) was added TEA (965.18 mg, 9.538 mmol, 2 equiv) dropwise at 0° C. The resulting mixture was stirred at room temperature for 12 h. The resulting mixture was concentrated under reduced pressure. The residue was purified by reversed-phase flash chromatography with the following conditions: column, C18 silica gel; mobile phase, MeCN in Water (0.1% FA), 10% to 50% gradient in 10 min; detector, UV 254 nm. This resulted in (2Z)-3-{[(3R)-1-(4-hydroxyphenyl)pyrrolidin-3-yl]carbamoyl}prop-2-enoic acid (700 mg, 53.13% yield) as a light yellow solid.

Step 5: A mixture of (2Z)-3-{[(3R)-1-(4-hydroxyphenyl)pyrrolidin-3-yl]carbamoyl}prop-2-enoic acid (600 mg, 2.172 mmol, 1 equiv) in AcOH (10 mL) was stirred at 70° C. for 12 h. The resulting mixture was concentrated under reduced pressure. The residue was purified by reversed-phase flash chromatography with the following conditions: column, C18 silica gel; mobile phase, MeCN in Water (0.1% FA), 10% to 60% gradient in 10 min; detector, UV 254 nm. This resulted in 1-[(3R)-1-(4-hydroxyphenyl)pyrrolidin-3-yl]pyrrole-2,5-dione (300 mg, 53.49% yield) as a light yellow solid.

Step 6: To a stirred mixture of 1-[(3R)-1-(4-hydroxyphenyl) pyrrolidin-3-yl]pyrrole-2,5-dione (300 mg, 1.162 mmol, 1 equiv) and 3-(fluorosulfonyl)-1,2-dimethylimidazol-1-ium triflate (457.53 mg, 1.394 mmol, 1.2 equiv) in DCM (3 mL) was added TEA (176.31 mg, 1.743 mmol, 1.5 equiv) in portions at 0° C. The resulting mixture was stirred at 0° C. for 30 min. The resulting mixture was concentrated under reduced pressure. The residue was purified by reversed-phase flash chromatography with the following conditions: column, C18 silica gel; mobile phase, MeCN in Water (0.1% FA), 10% to 95% gradient in 10 min; detector, UV 254 nm. This resulted in 4-[(3R)-3-(2,5-dioxopyrrol-1-yl)pyrrolidin-1-yl]phenyl sulfurofluoridate (100 mg, 25.30% yield, 99.7% purity) as a white solid. LCMS:(ES, m/z): [M+H]⁺=341.2

¹H NMR (300 MHz, DMSO-d₆, ppm) δ 7.36 (d, J=8.9 Hz, 2H), 7.04 (s, 2H), 6.61 (d, J=9.2 Hz, 2H), 4.77 (q, J=7.9 Hz, 1H), 3.59-3.40 (m, 4H), 2.48-2.17 (m, 4H).

### Example 59. Synthesis of 3-(4-(2,5-dioxo-2,5-dihydro-1H-pyrrol-1-yl)piperidin-1-yl)phenyl sulfurofluoridate (MFS-7-4)

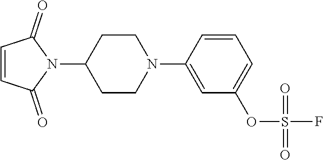 [SMILES: O=C1C=CC(=O)N1C1CCN(c2cccc(OS(=O)(=O)F)c2)CC1]

Step 1: A solution of tert-butyl N-(piperidin-4-yl) carbamate (3 g, 14.97 mmol, 1 equiv), Pd₂(dba)₃ (1.37 g, 1.49 mmol, 0.1 equiv), XPhos (1.43 g, 2.99 mmol, 0.2 equiv), t-BuONa (4.32 g, 44.93 mmol, 3 equiv) and 1-(benzyloxy)-3-bromobenzene (4.73 g, 17.97 mmol, 1.2 equiv) in toluene (30 mL) was stirred at 100° C. for overnight under nitrogen atmosphere. The mixture was allowed to cool down to room temperature. The resulting mixture was filtered, the filter cake was washed with MeCN (3×10 mL). The filtrate was concentrated under reduced pressure. The residue was purified by reversed-phase flash chromatography with the following conditions: column, C18 silica gel; mobile phase, MeCN in Water (0.1% FA), 10% to 50% gradient in 10 min; detector, UV 254 nm. This resulted in tert-butyl N-{1-[3-(benzyloxy) phenyl]piperidin-4-yl}carbamate (2 g, 34.91% yield, 95% purity) as a white solid.

Step 2: A solution of tert-butyl N-{1-[3-(benzyloxy) phenyl]piperidin-4-yl}carbamate (2 g, 5.22 mmol, 1 equiv) and Pd/C (0.56 g, 5.22 mmol, 1 equiv) in MeOH (20 mL) was stirred at room temperature for overnight under nitrogen atmosphere. The resulting mixture was filtered, the filter cake was washed with MeOH (3×5 mL). The filtrate was concentrated under reduced pressure. This resulted in tert-butyl N-[1-(3-hydroxyphenyl) piperidin-4-yl]carbamate (1.3 g, Crude Product) as a yellow oil.

Step 3: A solution of tert-butyl N-[1-(3-hydroxyphenyl) piperidin-4-yl]carbamate (1.3 g, 4.44 mmol, 1 equiv) in MeCN (15 mL) was treated with 1-(fluorosulfonyl)-2,3-dimethyl-1H-imidazol-3-ium trifluoromethanesulfonate (1.90 g, 5.78 mmol, 1.3 equiv) at 0° C. for 2 min under nitrogen atmosphere followed by the addition of TEA (1.35 g, 13.33 mmol, 3 equiv) dropwise at 0° C. The resulting mixture was stirred at 0° C. for 1 h under nitrogen atmosphere. The resulting mixture was concentrated under reduced pressure. The residue was purified by reversed-phase flash chromatography with the following conditions: column, C18 silica gel; mobile phase, MeCN in Water (0.1% FA), 10% to 50% gradient in 10 min; detector, UV 254 nm. This resulted in tert-butyl N-(1-{3-[(fluorosulfonyl)oxy]phenyl}piperidin-4-yl)carbamate (1.1 g, 66.07% yield, 95% purity) as a yellow oil.

Step 4: A solution of tert-butyl N-(1-{3-[(fluorosulfonyl)oxy]phenyl}piperidin-4-yl)carbamate (1.1 g, 2.93 mmol, 1 equiv) and TFA (5 mL) in DCM (5 mL) was stirred at room temperature for 2 h under nitrogen atmosphere. The resulting mixture was concentrated under reduced pressure. The residue was purified by reversed-phase flash chromatography with the following conditions: column, C18 silica gel; mobile phase, MeCN in Water (0.1% FA), 10% to 50% gradient in 10 min; detector, UV 254 nm. This resulted in 3-(4-aminopiperidin-1-yl)phenyl sulfurofluoridate (600 mg, 74.45% yield, 95% purity) as a yellow oil.

Step 5: A solution of 3-(4-aminopiperidin-1-yl) phenyl sulfurofluoridate (600 mg, 2.18 mmol, 1 equiv), 2,5-dihydrofuran-2,5-dione (428.96 mg, 4.37 mmol, 2 equiv) and TEA (664.02 mg, 6.56 mmol, 3 equiv) in MeCN (10 mL) was stirred at 80° C. for overnight under nitrogen atmosphere. The mixture was allowed to cool down to room temperature. The resulting mixture was concentrated under reduced pressure. The residue was purified by reversed-phase flash chromatography with the following conditions: column, C18 silica gel; mobile phase, MeCN in Water (0.1% FA), 10% to 50% gradient in 10 min; detector, UV 254 nm. This resulted in (2Z)-3-[(1-{3-[(fluorosulfonyl) oxy]phenyl}piperidin-4-yl) carbamoyl]prop-2-enoic acid (400 mg, 49.11% yield, 95% purity) as a yellow oil.

Step 6: A solution of 1-[1-(3-hydroxyphenyl) piperidin-4-yl]pyrrole-2,5-dione (70 mg, 0.25 mmol, 1 equiv) and 2,5-dihydrofuran-2,5-dione (0.06 g, 0.56 mmol, 3 equiv) in HOAc (5 mL) was stirred at 100° C. for overnight under nitrogen atmosphere. The mixture was allowed to cool down to room temperature. The resulting mixture was concentrated under reduced pressure. The residue was purified by silica gel column chromatography, eluted with PE/THF (10:1) to afford 3-[4-(2,5-dioxopyrrol-1-yl) piperidin-1-yl]phenyl sulfurofluoridate (13.3 mg, 19.97% yield, 99.3% purity) as a light yellow oil. LCMS:(ES, m/z): [M+H]⁺=355.0.

¹H NMR (300 MHz, DMSO-d₆, ppm) δ 7.37 (t, J=8.3 Hz, 1H), 7.14-7.05 (m, 2H), 6.98 (s, 2H), 6.92-6.80 (m, 1H), 4.11-3.99 (m, 1H), 3.91 (dt, J=13.0, 2.4 Hz, 2H), 2.85 (td, J=13.0, 2.5 Hz, 2H), 2.36-2.13 (m, 2H), 1.76-1.53 (m, 2H).

### Example 60. Synthesis of 3-((2-(2,5-dioxo-2,5-dihydro-1H-pyrrol-1-yl)ethyl)carbamoyl)phenyl sulfurofluoridate (MFS-7-5)

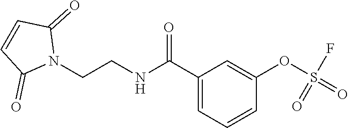 [SMILES: O=C(NCCN1C(=O)C=CC1=O)c1cccc(OS(=O)(=O)F)c1]

Step 1: A mixture of 3-(benzyloxy) benzoic acid (3.42 g, 14.980 mmol, 1.2 equiv), DIEA (4.84 g, 37.449 mmol, 3 equiv) and HATU (7.12 g, 18.724 mmol, 1.5 equiv) in DMF (40 mL) was stirred at room temperature for 30 min. To the above mixture was added tert-butyl N-(2-aminoethyl) carbamate (2 g, 12.483 mmol, 1 equiv) in portions at room temperature. The resulting mixture was stirred at room temperature for additional 1 h. The resulting mixture was extracted with EtOAc (3×40 mL). The combined organic layers were washed with water (3×20 mL), dried over anhydrous Na₂SO₄. After filtration, the filtrate was concentrated under reduced pressure. The residue was purified by reversed-phase flash chromatography with the following conditions: column, C18 silica gel; mobile phase, MeCN in Water (0.1% FA), 10% to 50% gradient in 10 min; detector, UV 254 nm. This resulted in tert-butyl N-(2-{[3-(benzyloxy) phenyl]formamido}ethyl)carbamate (2.2 g, 62.87% yield) as a light yellow solid.

Step 2: A mixture of tert-butyl N-(2-{[3-(benzyloxy) phenyl]formamido}ethyl) carbamate (2.2 g, 5.939 mmol, 1 equiv) in DCM (9 mL) and HCl in 1,4-dioxane (4.0 M) (3 mL) was stirred at room temperature for 2 h. The resulting mixture was concentrated under vacuum to afford N-(2-aminoethyl)-3-(benzyloxy) benzamide (1.5 g, 93.43% yield) as a white solid. The crude resulting mixture was used in the next step directly without further purification.

Step 3: To a solution of N-(2-aminoethyl)-3-(benzyloxy) benzamide (1.5 g, 5.549 mmol, 1 equiv) in 20 mL MeOH was added Pd/C (10%, 0.15 g) under nitrogen atmosphere in a 100 mL round-bottom flask. The mixture was hydrogenated at room temperature for 2 h under hydrogen atmosphere using a hydrogen balloon, filtered through a Celite pad and concentrated under reduced pressure to afford N-(2-aminoethyl)-3-hydroxybenzamide (900 mg, 90.01% yield) as a light yellow oil.

Step 4: To a stirred mixture of N-(2-aminoethyl)-3-hydroxybenzamide (900 mg, 4.994 mmol, 1 equiv) and maleic anhydride (734.58 mg, 7.491 mmol, 1.5 equiv) in ACN (10 mL) was added TEA (1010.77 mg, 9.988 mmol, 2 equiv) dropwise at 0° C. The resulting mixture was stirred at room temperature for 4 h. The resulting mixture was concentrated under reduced pressure. The residue was purified by reversed-phase flash chromatography with the following conditions: column, C18 silica gel; mobile phase, MeCN in Water (0.1% FA), 10% to 50% gradient in 10 min; detector, UV 254 nm. This resulted in (2Z)-3-({2-[(3-hydroxyphenyl)formamido]ethyl}carbamoyl)prop-2-enoic acid (800 mg, 57.57% yield) as a light yellow solid.

Step 5: A mixture of (2Z)-3-({2-[(3-hydroxyphenyl) formamido]ethyl}carbamoyl) prop-2-enoic acid (500 mg, 1.797 mmol, 1 equiv) in AcOH (5 mL) was stirred at 120° C. for 12 h. The resulting mixture was concentrated under reduced pressure. The residue was purified by reversed-phase flash chromatography with the following conditions: column, C18 silica gel; mobile phase, MeCN in Water (0.1% FA), 10% to 60% gradient in 10 min; detector, UV 254 nm. This resulted in N-[2-(2,5-dioxopyrrol-1-yl) ethyl]-3-hydroxybenzamide (300 mg, 64.15% yield) as a yellow solid.

Step 6: To a stirred mixture of N-[2-(2,5-dioxopyrrol-1-yl) ethyl]-3-hydroxybenzamide (200 mg, 0.768 mmol, 1 equiv) and 3-(fluorosulfonyl)-1,2-dimethylimidazol-1-ium triflate (302.71 mg, 0.922 mmol, 1.2 equiv) in DCM (5 mL) was added TEA (116.65 mg, 1.152 mmol, 1.5 equiv) in portions at 0° C. The resulting mixture was stirred at 0° C. for 30 min. The residue was purified by silica gel column chromatography, eluted with PE/EA (5:1) to afford 3-{[2-(2,5-dioxopyrrol-1-yl) ethyl]carbamoyl}phenyl sulfurofluoridate (100 mg, 38.01% yield, 99.4% purity) as a white solid. LCMS:(ES, m/z): [M+H]⁺=343.1.

¹H NMR (400 MHz, DMSO-d₆, ppm) δ 8.80 (t, J=6.0 Hz, 1H), 7.88 (dd, J=7.1, 1.5 Hz, 2H), 7.81-7.75 (m, 1H), 7.70 (t, J=8.1 Hz, 1H), 7.02 (s, 2H), 3.60 (dd, J=6.5, 4.8 Hz, 2H), 3.42 (q, J=5.9 Hz, 2H).

### Example 61. Synthesis of 4-(4-(2,5-dioxo-2,5-dihydro-1H-pyrrol-1-yl)piperidin-1-yl)phenyl sulfurofluoridate (MFS-8-2)

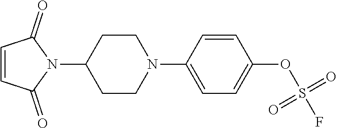 [SMILES: O=C1C=CC(=O)N1C1CCN(c2ccc(OS(=O)(=O)F)cc2)CC1]

Step 1: To a stirred mixture of tert-butyl N-(piperidin-4-yl) carbamate (1 g, 4.993 mmol, 1 equiv) and 1-(benzyloxy)-4-bromobenzene (1.31 g, 4.993 mmol, 1 equiv) in toluene (5 mL) was added t-BuONa (1.20 g, 12.483 mmol, 2.5 equiv) in portions at room temperature. To the above mixture was added Pd₂(dba)₃ (0.46 g, 0.499 mmol, 0.1 equiv) and XPhos (0.48 g, 0.999 mmol, 0.2 equiv) under nitrogen atmosphere. The resulting mixture was stirred at 60° C. for additional overnight. The reaction was monitored by LCMS. The resulting mixture was extracted with EtOAc (4×2 mL). The combined organic layers were washed with water (3×2 mL), dried over anhydrous Na₂SO₄. After filtration, the filtrate was concentrated under reduced pressure. The residue was purified by Prep-TLC (PE/EA 5:1) to afford tert-butyl N-{1-[4-(benzyloxy) phenyl]piperidin-4-yl}carbamate (1.5 g, 78.54% yield) as a white solid.

Step 2: A solution of tert-butyl N-{1-[4-(benzyloxy) phenyl]piperidin-4-yl}carbamate (1.4 g, 3.660 mmol, 1 equiv) in EtOH (4 mL) was treated with Pd/C at room temperature for 30 min under hydrogen atmosphere. The reaction was monitored by LCMS. The resulting mixture was filtered, the filter cake was washed with EtOH (4 mL) (2×1 mL). The filtrate was concentrated under reduced pressure. This resulted in tert-butyl N-[1-(4-hydroxyphenyl) piperidin-4-yl]carbamate (1 g, 93.45% yield) as a yellow solid.

Step 3: To a stirred mixture of tert-butyl N-[1-(4-hydroxyphenyl) piperidin-4-yl]carbamate (1 g, 3.420 mmol, 1 equiv) and 3-(fluorosulfonyl)-1,2-dimethylimidazol-1-ium triflate (1.35 g, 4.104 mmol, 1.2 equiv) in MeCN (4 mL) was added TEA (0.69 g, 6.840 mmol, 2 equiv) dropwise at 0° C. The reaction was monitored by LCMS. The crude product was purified by reverse phase flash to afford tert-butyl N-(1-{4-[(fluorosulfonyl) oxy]phenyl}piperidin-4-yl) carbamate (540 mg, 42.17% yield) as a yellow oil.

Step 4: A solution of tert-butyl N-(1-{4-[(fluorosulfonyl) oxy]phenyl}piperidin-4-yl)carbamate (540 mg, 1.442 mmol, 1 equiv) in DCM (1 mL) was treated with HCl in 1,4-dioxane (4.0 M) (1 mL) at room temperature for 1 h. The reaction was monitored by LCMS. The resulting mixture was concentrated under reduced pressure. The residue was dissolved in DCM (1 mL). The residue was purified by Prep-TLC (PE/EA 5:1) to afford 4-(4-aminopiperidin-1-yl) phenyl sulfurofluoridate (470 mg, 94.88% yield) as a pink solid.

Step 5: To a stirred mixture of 4-(4-aminopiperidin-1-yl) phenyl sulfurofluoridate (430 mg, 1.568 mmol, 1 equiv) and maleic anhydride (153.71 mg, 1.568 mmol, 1 equiv) in THF (3 mL) was added TEA (317.25 mg, 3.136 mmol, 2 equiv) dropwise at 0° C. The reaction was monitored by LCMS. The resulting mixture was concentrated under reduced pressure. The residue product was purified by reverse phase flash to afford (2Z)-3-[(1-{4-[(fluorosulfonyl)oxy]phenyl}piperidin-4-yl) carbamoyl]prop-2-enoic acid (400 mg, 68.53% yield) as a yellow solid.

Step 6: A mixture of (2Z)-3-[(1-{4-[(fluorosulfonyl) oxy]phenyl}piperidin-4-yl) carbamoyl]prop-2-enoic acid (100 mg, 0.269 mmol, 1 equiv) in acetic acid was stirred at 100° C. for 16 h under nitrogen atmosphere. The residue was purified by silica gel column chromatography, eluted with MeOH/H₂O (5:1) to afford 4-[4-(2,5-dioxopyrrol-1-yl) piperidin-1-yl]phenyl sulfurofluoridate, (20 mg, 20.96% yield, 98% purity) as a white solid. LCMS:(ES, m/z): [M]⁺=355.1.

¹H NMR (300 MHz, DMSO-d₆, ppm) δ 7.39 (d, J=9.1 Hz, 2H), 7.08 (d, J=9.4 Hz, 2H), 7.00 (s, 2H), 4.08-3.98 (m, 1H), 3.87 (d, J=12.2 Hz, 2H), 2.83 (t, J=11.7 Hz, 2H), 2.27-2.19 (m, 2H), 1.68 (d, J=12.9 Hz, 2H).

### Example 62. Synthesis of 4-((2-(2,5-dioxo-2,5-dihydro-1H-pyrrol-1-yl)ethyl)carbamoyl)phenyl sulfurofluoridate (MFS-8-3)

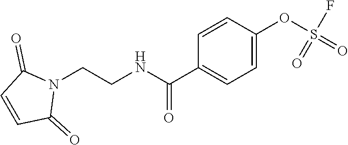 [SMILES: O=C(NCCN1C(=O)C=CC1=O)c1ccc(OS(=O)(=O)F)cc1]

Step 1: To a stirred solution of tert-butyl N-(2-aminoethyl) carbamate (3 g, 18.725 mmol, 1 equiv) and T₃P (8.94 g, 28.088 mmol, 1.5 equiv) in THF (50 mL) were added P-hydroxybenzoic acid (2.59 g, 18.725 mmol, 1 equiv) and DIEA (7.26 g, 56.175 mmol, 3 equiv) in portions at 0° C. The resulting mixture was stirred at room temperature for additional 2 h. The reaction was monitored by LCMS. The resulting mixture was concentrated under reduced pressure. The residue was purified by reversed-phase flash chromatography with the following conditions: column, C18 silica gel; mobile phase, MeCN in Water (0.1% NH₃·H₂O), 10% to 60% gradient in 8 min; detector, UV 254 nm. This resulted in tert-butyl N-{2-[(4-hydroxyphenyl) formamido]ethyl}carbamate (3 g, 57.15% yield) as a colorless oil.

Step 2: To a stirred solution of tert-butyl N-{2-[(4-hydroxyphenyl) formamido]ethyl}carbamate (3 g, 10.702 mmol, 1 equiv) in DCM (10 mL) was added HCl/1,4-dioxane (5 mL) dropwise at 0° C. The resulting mixture was stirred at room temperature for additional 1 h. The reaction was monitored by LCMS. The resulting mixture was concentrated under reduced pressure. The residue was purified by trituration with Et₂O (5 mL). This resulted in N-(2-aminoethyl)-4-hydroxybenzamide hydrochloride (1.3 g, 56.06% yield) as a white solid.

Step 3: To a stirred mixture of N-(2-aminoethyl)-4-hydroxybenzamide hydrochloride (1.3 g, 6.000 mmol, 1 equiv) and maleic anhydride (1.18 g, 12.000 mmol, 2 equiv) was added AcOH (15 mL) dropwise at room temperature. The resulting mixture was stirred at 100° C. for additional overnight. The reaction was monitored by LCMS. The resulting mixture was concentrated under reduced pressure. The residue was purified by reversed-phase flash chromatography with the following conditions: column, C18 silica gel; mobile phase, MeCN in Water (0.1% FA), 10% to 60% gradient in 10 min; detector, UV 254 nm. This resulted in N-[2-(2,5-dioxopyrrol-1-yl) ethyl]-4-hydroxybenzamide (240 mg, 15.37% yield) as a white solid.

Step 4: To a stirred mixture of N-[2-(2,5-dioxopyrrol-1-yl) ethyl]-4-hydroxybenzamide (240 mg, 0.922 mmol, 1 equiv) and 3-(fluorosulfonyl)-1,2-dimethylimidazol-1-ium triflate (363.25 mg, 1.106 mmol, 1.2 equiv) in ACN (8 mL) was added TEA (186.64 mg, 1.844 mmol, 2 equiv) dropwise at 0° C. The resulting mixture was stirred at room temperature for additional 30 min. The reaction was monitored by LCMS. The residue was purified by reversed-phase flash chromatography with the following conditions: column, C18 silica gel; mobile phase, MeCN in Water (0.1% FA), 10% to 70% gradient in 12 min; detector, UV 254 nm. This resulted in 4-{[2-(2,5-dioxopyrrol-1-yl) ethyl]carbamoyl}phenyl sulfurofluoridate (53 mg, 16.79% yield, 96.8% purity) as a white solid. LCMS: (ES, m/z): [M+H]⁺=343.1.

¹H NMR (300 MHz, DMSO-d₆, ppm) δ 8.74 (t, J=6.0 Hz, 1H), 7.95-7.84 (m, 2H), 7.76-7.66 (m, 2H), 7.00 (s, 2H), 3.57 (dd, J=6.6, 4.6 Hz, 2H), 3.41 (d, J=5.9 Hz, 2H).

### Example 63. Synthesis of 4-((3-(2,5-dioxo-2,5-dihydro-1H-pyrrol-1-yl)propyl)carbamoyl)phenyl sulfurofluoridate (MFS-9-4)

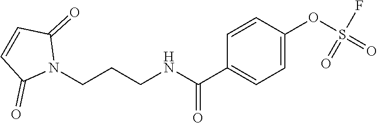 [SMILES: O=C(NCCCN1C(=O)C=CC1=O)c1ccc(OS(=O)(=O)F)cc1]

Step 1: To a stirred solution of tert-butyl N-(3-aminopropyl) carbamate (3 g, 17.217 mmol, 1 equiv) and T3P (8.22 g, 25.825 mmol, 1.5 equiv) in THF (50 mL) were added P-hydroxybenzoic acid (2.38 g, 17.217 mmol, 1 equiv) and DIEA (6.68 g, 51.651 mmol, 3 equiv) in portions at 0° C. The resulting mixture was stirred at room temperature for additional 2 h. The reaction was monitored by LCMS. The resulting mixture was concentrated under reduced pressure. The residue was purified by reversed-phase flash chromatography with the following conditions: column, C18 silica gel; mobile phase, MeCN in Water (0.1% NH3·H2O), 10% to 60% gradient in 8 min; detector, UV 254 nm. This resulted in tert-butyl N-{3-[(4-hydroxyphenyl) formamido]propyl}carbamate (1.8 g, 35.52% yield) as a white solid.

Step 2: To a stirred mixture of tert-butyl N-{3-[(4-hydroxyphenyl) formamido]propyl}carbamate (1.8 g, 6.115 mmol, 1 equiv) in DCM (10 mL) was added TFA (5 mL) dropwise at 0° C. The resulting mixture was stirred at room temperature for additional 1 h. The reaction was monitored by LCMS. The resulting mixture was concentrated under reduced pressure. The residue was purified by trituration with Et₂O (5 mL). This resulted in N-(3-aminopropyl)-4-hydroxybenzamide (1 g, 84.19% yield) as a white solid.

Step 3: To a stirred mixture of N-(3-aminopropyl)-4-hydroxybenzamide (1 g, 5.148 mmol, 1.00 equiv) and maleic anhydride (1.01 g, 10.296 mmol, 2 equiv) was added AcOH (12 mL) dropwise at room temperature. The resulting mixture was stirred at 100° C. for additional overnight. The reaction was monitored by LCMS. The resulting mixture was concentrated under reduced pressure. The residue was purified by reversed-phase flash chromatography with the following conditions: column, C18 silica gel; mobile phase, MeCN in Water (0.1% FA), 10% to 60% gradient in 10 min; detector, UV 254 nm. This resulted in N-[3-(2,5-dioxopyrrol-1-yl) propyl]-4-hydroxybenzamide (260 mg, 18.41% yield) as a colorless oil.

Step 4: To a stirred mixture of N-[3-(2,5-dioxopyrrol-1-yl) propyl]-4-hydroxybenzamide (250 mg, 0.911 mmol, 1.00 equiv) and 3-(fluorosulfonyl)-1,2-dimethylimidazol-1-ium triflate (448.80 mg, 1.367 mmol, 1.50 equiv) in DCM (10 mL) was added TEA (184.47 mg, 1.822 mmol, 2.0 equiv) dropwise at 0° C. The resulting mixture was stirred at room temperature for additional 30 min. The reaction was monitored by LCMS. The residue was purified by silica gel column chromatography, eluted with PE/EA (4:1) to afford 4-{[3-(2,5-dioxopyrrol-1-yl) propyl]carbamoyl}phenyl sulfurofluoridate (122 mg, 37.56% yield, 97.3% purity) as a light yellow solid. LCMS: (ES, m/z): [M+H]⁺=357.1.

¹H NMR (300 MHz, DMSO-d₆, ppm) δ 8.66 (t, J=5.7 Hz, 1H), 8.09-7.94 (m, 2H), 7.80-7.66 (m, 2H), 7.03 (s, 2H), 3.47 (t, J=7.2 Hz, 2H), 3.32-3.18 (m, 2H), 1.78 (p, J=7.2 Hz, 2H).

### Example 64. Synthesis of 4-((aminooxy)methyl)phenyl sulfurofluoridate hydrochloride (PFS-5-1)

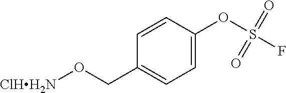

Step 1: A solution of gastrodigenin (500 mg, 4.02 mmol, 1 equiv), Benzyl bromide (826.67 mg, 4.83 mmol, 1.2 equiv) and K₂CO₃ (1113.31 mg, 8.05 mmol, 2 equiv) in acetone (10 mL) was stirred at 50° C. for overnight under nitrogen atmosphere. The mixture was allowed to cool down to room temperature. The resulting mixture was filtered, the filter cake was washed with acetone (3×5 mL). The filtrate was concentrated under reduced pressure. This resulted in 4-benzyloxybenzyl alcohol (1 g, Crude Product) as a yellow solid.

Step 2: To a stirred solution of 4-benzyloxybenzyl alcohol (1 g, 4.66 mmol, 1 equiv) in DCM (10 mL) was added PBr3 (1.39 g, 5.13 mmol, 1.1 equiv) dropwise at 0° C. under nitrogen atmosphere. The resulting mixture was stirred at 0° C. for 2 h under nitrogen atmosphere. The reaction was quenched by the addition of water (10 mL) at 0° C. The resulting mixture was extracted with CH₂Cl₂ (2×10 mL). The combined organic layers were washed with brine (1×10 mL), dried over anhydrous Na₂SO₄. After filtration, the filtrate was concentrated under reduced pressure. The residue was purified by silica gel column chromatography, eluted with PE/EA (5:1) to afford 1-(benzyloxy)-4-(bromomethyl)benzene (1 g, 77.31% yield, 95% purity) as a yellow oil.

Step 3: A solution of 1-(benzyloxy)-4-(bromomethyl) benzene (1 g, 3.60 mmol, 1 equiv), DBU (0.55 g, 3.60 mmol, 1 equiv) and tert-butyl N-hydroxycarbamate (0.58 g, 4.33 mmol, 1.2 equiv) in ACN (10 mL) was stirred at 60° C. for 2 h under nitrogen atmosphere. The mixture was allowed to cool down to room temperature. The resulting mixture was concentrated under reduced pressure. The residue was purified by reversed-phase flash chromatography with the following conditions: column, C18 silica gel; mobile phase, MeCN in Water (0.1% FA), 10% to 50% gradient in 10 min; detector, UV 254 nm. This resulted in tert-butyl N-{[4-(benzyloxy)phenyl]methoxy}carbamate (1 g, 84.14% yield, 95% purity) as a yellow oil.

Step 4: A solution of tert-butyl N-{[4-(benzyloxy) phenyl]methoxy}carbamate (1 g, 3.03 mmol, 1 equiv) and Pd/C (0.16 g, 1.51 mmol, 0.5 equiv) in MeOH (20 mL) was stirred at room temperature for 30 min under hydrogen atmosphere. The resulting mixture was filtered, the filter cake was washed with MeOH (3×4 mL). The filtrate was concentrated under reduced pressure. This resulted in tert-butyl N-[(4-hydroxyphenyl)methoxy]carbamate (900 mg, Crude Product) as a yellow oil.

Step 5: A solution of tert-butyl N-[(4-hydroxyphenyl) methoxy]carbamate (900 mg, 3.76 mmol, 1 equiv) in MeCN (10 mL) was treated with 3-(fluorosulfonyl)-1,2-dimethylimidazol-1-ium triflate (1605.09 mg, 4.89 mmol, 1.3 equiv) at 0° C. for 3 min under nitrogen atmosphere followed by the addition of TEA (1141.89 mg, 11.28 mmol, 3 equiv) dropwise at 0° C. The resulting mixture was concentrated under reduced pressure. The residue was purified by silica gel column chromatography, eluted with PE/EA (5:1) to afford tert-butyl N-({4-[(fluorosulfonyl)oxy]phenyl}methoxy)carbamate (400 mg, 33.10% yield, 95% purity) as a yellow oil.

Step 6: A solution of tert-butyl N-({4-[(fluorosulfonyl) oxy]phenyl}methoxy) carbamate (300 mg, 0.93 mmol, 1 equiv) in hydrogen chloride (2.0 M in diethyl ether) (10 mL) was stirred at room temperature for 2 days under nitrogen atmosphere. The precipitated solids were collected by filtration and washed with diethyl ether (3×3 mL). This resulted in 4-[(aminooxy)methyl]phenyl sulfurofluoridate hydrochloride (53.4 mg, 22.20% yield, 98.4% purity) as a white solid. LCMS:(ES, m/z): [M+H]⁺=222.0.

¹H NMR (300 MHz, DMSO-d₆, ppm) δ 11.07 (s, 2H), 7.72-7.60 (m, 4H), 5.10 (s, 2H).

### Example 65. Synthesis of 3-(2-(aminooxy)ethoxy)phenyl sulfurofluoridate hydrochloride (PFS-6-1)

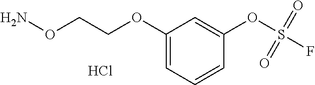 [SMILES: NOCCOc1cccc(OS(=O)(=O)F)c1 ; Cl]

Step 1: A solution of resorcinol (6 g, 54.49 mmol, 1 equiv), K₂CO₃ (11.30 g, 81.73 mmol, 1.5 equiv) and benzyl bromide (7.46 g, 43.59 mmol, 0.8 equiv) in acetone (60 mL) was stirred at room temperature for overnight under nitrogen atmosphere. The resulting mixture was concentrated under reduced pressure. The residue was purified by reversed-phase flash chromatography with the following conditions: column, C18 silica gel; mobile phase, MeCN in Water (0.1% FA), 10% to 50% gradient in 10 min; detector, UV 254 nm. This resulted in 3-(benzyloxy) phenol (3.2 g, 29.33% yield, 95% purity) as a yellow oil.

Step 2: A solution of 3-(benzyloxy) phenol (3.2 g, 15.981 mmol, 1 equiv), dibromoethane (18.01 g, 95.88 mmol, 6 equiv) and K₂CO₃ (5.52 g, 39.95 mmol, 2.5 equiv) in MeCN (35 mL) was stirred at 100° C. for overnight under nitrogen atmosphere. The mixture was allowed to cool down to room temperature. The resulting mixture was concentrated under reduced pressure. The residue was purified by reversed-phase flash chromatography with the following conditions: column, C18 silica gel; mobile phase, MeCN in Water (0.1% FA), 10% to 50% gradient in 10 min; detector, UV 254 nm. This resulted in 1-(benzyloxy)-3-(2-bromoethoxy) benzene (1.3 g, 26.48% yield, 95% purity) as a yellow oil.

Step 3: A solution of 1-(benzyloxy)-3-(2-bromoethoxy) benzene (1.3 g, 4.23 mmol, 1 equiv), tert-butyl N-hydroxycarbamate (0.68 g, 5.07 mmol, 1.2 equiv) and DBU (0.64 g, 4.23 mmol, 1 equiv) in MeCN (15 mL) was stirred at 60° C. for 1 h under nitrogen atmosphere. The mixture was allowed to cool down to room temperature. The resulting mixture was concentrated under reduced pressure. The residue was purified by reversed-phase flash chromatography with the following conditions: column, C18 silica gel; mobile phase, MeCN in Water (0.1% FA), 10% to 50% gradient in 10 min; detector, UV 254 nm. This resulted in tert-butyl N-{2-[3-(benzyloxy)phenoxy]ethoxy}carbamate (770 mg, 50.62% yield, 95% purity) as a yellow oil.

Step 4: A solution of tert-butyl N-{2-[3-(benzyloxy)phenoxy]ethoxy}carbamate (770 mg, 2.14 mmol, 1 equiv) and Pd/C (113.99 mg, 1.07 mmol, 0.5 equiv) in MeOH (10 mL) was stirred at room temperature for 1 h under nitrogen atmosphere. The resulting mixture was filtered, the filter cake was washed with MeOH (3×4 mL). The filtrate was concentrated under reduced pressure. This resulted in tert-butyl N-[2-(3-hydroxyphenoxy) ethoxy]carbamate (600 mg, Crude Product) as a yellow oil.

Step 5: A solution of tert-butyl N-[2-(3-hydroxyphenoxy) ethoxy]carbamate (600 mg, 2.22 mmol, 1 equiv) in MeCN (10 mL) was treated with 1-(fluorosulfonyl)-2,3-dimethyl-1H-imidazol-3-ium trifluoromethanesulfonate (950.75 mg, 2.89 mmol, 1.3 equiv) at 0° C. for 3 min under nitrogen atmosphere followed by the addition of TEA (676.38 mg, 6.68 mmol, 3 equiv) dropwise at 0° C. The resulting mixture was stirred at 0° C. for 1 h under nitrogen atmosphere. The resulting mixture was concentrated under reduced pressure. The residue was purified by reversed-phase flash chromatography with the following conditions: column, C18 silica gel; mobile phase, MeCN in Water (0.1% FA), 10% to 50% gradient in 10 min; detector, UV 254 nm. This resulted in tert-butyl N-(2-{3-[(fluorosulfonyl) oxy]phenoxy}ethoxy) carbamate (500 mg, 63.87% yield, 95% purity) as a yellow oil.

Step 6: A solution of tert-butyl N-(2-{3-[(fluorosulfonyl) oxy]phenoxy}ethoxy) carbamate (300 mg, 0.85 mmol, 1 equiv) in hydrogen chloride (2.0 M in diethyl ether) (10 mL) was stirred at room temperature for overnight under nitrogen atmosphere. The precipitated solids were collected by filtration and washed with diethyl ether (3×5 mL). This resulted in 3-[2-(aminooxy) ethoxy]phenyl sulfurofluoridate hydrochloride (54.2 mg, 22.06% yield, 96.8% purity) as a white solid. LCMS:(ES, m/z): [M+H]⁺=252.0.

¹H NMR (300 MHz, DMSO-d₆, ppm) δ 11.03 (s, 2H), 7.53 (t, J=8.3 Hz, 1H), 7.26 (t, J=2.5 Hz, 1H), 7.23-7.10 (m, 2H), 4.37 (dd, J=6.0, 2.4 Hz, 2H), 4.32 (dd, J=6.0, 2.3 Hz, 2H).

### Example 66. Synthesis of 4-(2-(aminooxy)ethoxy)phenyl sulfurofluoridate hydrochloride (PFS-7-1)

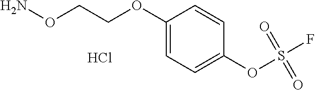 [SMILES: NOCCOc1ccc(OS(=O)(=O)F)cc1 ; Cl]

Step 1: A solution of monobenzone (3 g, 14.98 mmol, 1 equiv), dibromoethane (16.89 g, 89.89 mmol, 6 equiv) and K₂CO₃ (5.18 g, 37.45 mmol, 2.5 equiv) in MeCN (30 mL) was stirred at 100° C. for overnight under nitrogen atmosphere. The mixture was allowed to cool down to room temperature. The resulting mixture was concentrated under reduced pressure. The residue was purified by reversed-phase flash chromatography with the following conditions: column, C18 silica gel; mobile phase, MeCN in Water (0.1% FA), 10% to 50% gradient in 10 min; detector, UV 254 nm. This resulted in 1-(benzyloxy)-4-(2-bromoethoxy) benzene (1.8 g, 39.11% yield, 95% purity) as a yellow oil.

Step 2: A solution of 1-(benzyloxy)-4-(2-bromoethoxy) benzene (1.8 g, 5.86 mmol, 1 equiv), tert-butyl N-hydroxycarbamate (0.94 g, 7.03 mmol, 1.2 equiv) and DBU (0.89 g, 5.86 mmol, 1 equiv) in MeCN (20 mL) was stirred at 60° C. for 2 h under nitrogen atmosphere. The mixture was allowed to cool down to room temperature. The resulting mixture was concentrated under reduced pressure. The residue was purified by reversed-phase flash chromatography with the following conditions: column, C18 silica gel; mobile phase, MeCN in Water (0.1% FA), 10% to 50% gradient in 10 min; detector, UV 254 nm. This resulted in tert-butyl N-{2-[4-(benzyloxy)phenoxy]ethoxy}carbamate (670 mg, 31.81% yield, 95% purity) as a yellow oil.

Step 3: A solution of tert-butyl N-{2-[4-(benzyloxy)phenoxy]ethoxy}carbamate (670 mg, 1.86 mmol, 1 equiv) and Pd/C (99.19 mg, 0.93 mmol, 0.5 equiv) in MeOH (10 mL) was stirred at room temperature for 1 h under nitrogen atmosphere. The resulting mixture was filtered, the filter cake was washed with MeOH (3×3 mL). The filtrate was concentrated under reduced pressure. This resulted in tert-butyl N-[2-(4-hydroxyphenoxy) ethoxy]carbamate (500 mg, Crude Product) as a yellow oil.

Step 4: A solution of tert-butyl N-[2-(4-hydroxyphenoxy) ethoxy]carbamate (500 mg, 1.85 mmol, 1 equiv) in MeCN (5 mL) was treated with 1-(fluorosulfonyl)-2,3-dimethyl-1H-imidazol-3-ium trifluoromethanesulfonate (792.29 mg, 2.41 mmol, 1.3 equiv) at 0° C. for 3 min under nitrogen atmosphere followed by the addition of TEA (375.77 mg, 3.71 mmol, 2 equiv) dropwise in portions at 0° C. The resulting mixture was stirred at 0° C. for 1 h under nitrogen atmosphere. The resulting mixture was concentrated under reduced pressure. The residue was purified by reversed-phase flash chromatography with the following conditions: column, C18 silica gel; mobile phase, MeCN in Water (0.1% FA), 10% to 50% gradient in 10 min; detector, UV 254 nm. This resulted in tert-butyl N-(2-{4-[(fluorosulfonyl) oxy]phenoxy}ethoxy) carbamate (250 mg, 38.32% yield, 95% purity) as a yellow oil.

Step 5: A solution of tert-butyl N-(2-{4-[(fluorosulfonyl) oxy]phenoxy}ethoxy) carbamate (150 mg, 0.42 mmol, 1 equiv) in hydrogen chloride (2.0 M in diethyl ether) (10 mL) was stirred at room temperature for 2 days under nitrogen atmosphere. The precipitated solids were collected by filtration and washed with diethyl ether (3×3 mL). This resulted in 4-[2-(aminooxy) ethoxy]phenyl sulfurofluoridate hydrochloride (50.4 mg, 41.04% yield, 98.5% purity) as a white solid. LCMS:(ES, m/z): [M+H]⁺=252.0.

¹H NMR (300 MHz, DMSO-d₆, ppm) δ 11.00 (s, 2H), 7.61-7.49 (m, 2H), 7.20-7.08 (m, 2H), 4.64-3.96 (m, 4H).

### Example 67. Synthesis of 3-(2-(2,5-dioxo-2,5-dihydro-1H-pyrrol-1-yl)ethyl)phenyl sulfurofluoridate (MFS-5-2)

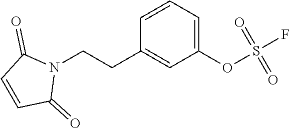 [SMILES: O=C1C=CC(=O)N1CCc1cccc(OS(=O)(=O)F)c1]

Step 1: A mixture of m-tyramine (2 g, 14.579 mmol, 1 equiv) and maleic anhydride (1715.51 mg, 17.495 mmol, 1.2 equiv) in AcOH (5 mL) was stirred at 100° C. for 12 h. The reaction was monitored by LCMS. The resulting mixture was concentrated under reduced pressure. The crude product was purified by reverse phase flash to afford 1-[2-(3-hydroxyphenyl) ethyl]pyrrole-2,5-dione (1.2 g, 37.89% yield) as a white solid.

Step 2: To a stirred solution of 3-(fluorosulfonyl)-1,2-dimethylimidazol-1-ium triflate (1.81 g, 5.524 mmol, 1 equiv) and 1-[2-(3-hydroxyphenyl) ethyl]pyrrole-2,5-dione (1.2 g, 5.524 mmol, 1.00 equiv) in MeCN was added TEA (1.12 g, 11.048 mmol, 2 equiv) dropwise at 0° C. under nitrogen atmosphere. The resulting mixture was stirred at 0° C. for 10 min under nitrogen atmosphere. The reaction was monitored by TLC. The resulting mixture was concentrated under vacuum. The residue was purified by reversed-phase flash chromatography with the following conditions: column, C18 silica gel; mobile phase, MeCN in Water (0.1% FA), 10% to 50% gradientin 10 min; detector, UV 254 nm. This resulted in 3-[2-(2,5-dioxopyrrol-1-yl) ethyl]phenyl sulfurofluoridate (300 mg, 18.15% yield, 99.8% purity) as a white solid. GCMS: (ES, m/z): [M]⁺=299.0.

¹H NMR (300 MHz, DMSO-d₆, ppm) δ 7.54-7.39 (m, 3H), 7.33 (dt, J=7.5, 1.4 Hz, 1H), 6.97 (s, 2H), 3.69 (t, J=6.9 Hz, 2H), 2.92 (t, J=6.9 Hz, 2H).

### Example 68. Synthesis of 4-(4-(2-(2,5-dioxo-2,5-dihydro-1H-pyrrol-1-yl)ethyl)piperidin-1-yl)phenyl sulfurofluoridate (MFS-10-1)

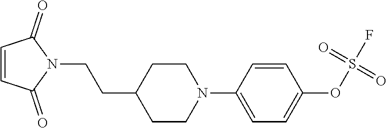 [SMILES: O=C1C=CC(=O)N1CCC1CCN(c2ccc(OS(=O)(=O)F)cc2)CC1]

Step 1: To a stirred solution of tert-butyl N-[2-(piperidin-4-yl)ethyl]carbamate (3 g, 13.139 mmol, 1 equiv), 1-(benzyloxy)-4-bromobenzene (5.19 g, 19.709 mmol, 1.5 equiv), Pd₂(dba)₃ (1.20 g, 1.314 mmol, 0.1 equiv), XPhos (1.25 g, 2.628 mmol, 0.2 equiv) and t-BuONa (3.79 g, 39.417 mmol, 3 equiv) in toluene (40 mL) at room temperature under nitrogen atmosphere. The resulting mixture was stirred at 100° C. for additional overnight. The mixture was allowed to cool down to room temperature. The resulting mixture was diluted with 40 mL H₂O and extracted with EtOAc (40 mL×3). The combined organic layers were washed with brine (40 mL), dried over anhydrous Na₂SO₄. After filtration, the filtrate was concentrated under reduced pressure. The residue was purified by silica gel column chromatography, eluted with PE/THF (3:1) to afford tert-butyl N-(2-{1-[4-(benzyloxy)phenyl]piperidin-4-yl}ethyl)carbamate (1.8 g, 33.37% yield) as a yellow solid.

Step 2: To a stirred solution of tert-butyl N-(2-{1-[4-(benzyloxy) phenyl]piperidin-4-yl}ethyl)carbamate (1.8 g, 4.384 mmol, 1 equiv) in DCM (10 mL) was added HCl in 1,4-dioxane (4.0 M) (10 mL) dropwise at room temperature under nitrogen atmosphere. The resulting mixture was stirred at room temperature for additional 2 h. The precipitated solids were collected by filtration and washed with DCM (5 mL×3). This resulted in 2-{1-[4-(benzyloxy) phenyl]piperidin-4-yl}ethanamine (1.3 g, 95.51% yield) as a light yellow solid. The crude product was used in the next step directly without further purification.

Step 3: To a stirred solution of 2-{1-[4-(benzyloxy) phenyl]piperidin-4-yl}ethanamine (1.3 g, 4.188 mmol, 1 equiv) and palladium (200 mg, 1.879 mmol, 0.45 equiv) in MeOH (15 mL) at room temperature under hydrogen atmosphere. The resulting mixture was stirred at room temperature for additional 2 h under hydrogen atmosphere. The resulting mixture was filtered, the filter cake was washed with MeOH (5 mL×3). This resulted in 4-[4-(2-aminoethyl) piperidin-1-yl]phenol (900 mg, 97.55% yield) as a light yellow oil. The crude product was used in the next step directly without further purification.

Step 4: To a stirred solution of 4-[4-(2-aminoethyl) piperidin-1-yl]phenol (900 mg, 4.085 mmol, 1 equiv) and maleic anhydride (480.68 mg, 4.902 mmol, 1.2 equiv) in MeCN (10 mL) was added TEA (826.76 mg, 8.170 mmol, 2 equiv) dropwise at 0° C. under nitrogen atmosphere. The resulting mixture was stirred at room temperature for additional 1 h. The resulting mixture was concentrated under reduced pressure. The residue was purified by reversed-phase flash chromatography with the following conditions: column, C18 silica gel; mobile phase, MeCN in Water (0.1% FA), 10% to 50% gradient in 10 min; detector, UV 254 nm. This resulted in (2Z)-3-({2-[1-(4-hydroxyphenyl) piperidin-4-yl]ethyl}carbamoyl)prop-2-enoic acid (600 mg, 46.13% yield) as a light yellow oil.

Step 5: To a stirred solution of (2Z)-3-({2-[1-(4-hydroxyphenyl) piperidin-4-yl]ethyl}carbamoyl) prop-2-enoic acid (600 mg, 1.885 mmol, 1 equiv) and maleic anhydride (277.19 mg, 2.828 mmol, 1.5 equiv) in HOAc (6 mL) at room temperature under nitrogen atmosphere. The resulting mixture was stirred at 100° C. for additional 16 h. The mixture was allowed to cool down to room temperature. The resulting mixture was concentrated under reduced pressure. The residue was purified by reversed-phase flash chromatography with the following conditions: column, C18 silica gel; mobile phase, MeCN in Water (0.1% FA), 10% to 50% gradient in 10 min; detector, UV 254 nm. This resulted in 1-{2-[1-(4-hydroxyphenyl) piperidin-4-yl]ethyl}pyrrole-2,5-dione (200 mg, 35.33% yield) as a yellow oil.

Step 6: To a stirred solution of 1-{2-[1-(4-hydroxyphenyl) piperidin-4-yl]ethyl}pyrrole-2,5-dione (300 mg, 0.999 mmol, 1 equiv) and 3-(fluorosulfonyl)-1,2-dimethylimidazol-1-ium triflate (393.43 mg, 1.199 mmol, 1.2 equiv) in MeCN (5 mL) was added TEA (202.14 mg, 1.998 mmol, 2 equiv) dropwise at 0° C. under nitrogen atmosphere. The resulting mixture was stirred at room temperature for additional 30 min. The resulting mixture was concentrated under reduced pressure. The residue was purified by reversed-phase flash chromatography with the following conditions: column, C18 silica gel; mobile phase, MeCN in Water (0.1% FA), 10% to 90% gradient in 10 min; detector, UV 254 nm. This resulted in 4-{4-[2-(2,5-dioxopyrrol-1-yl) ethyl]piperidin-1-yl}phenyl sulfurofluoridate (50 mg, 13.09% yield, 99.3% purity) as a light yellow oil. LCMS:(ES, m/z): [M+H]⁺=383.1.

¹H NMR (300 MHz, DMSO-d₆, ppm) δ 7.35 (d, J=8.9 Hz, 2H), 7.07-6.99 (m, 4H), 3.74 (d, J=12.5 Hz, 2H), 3.46 (t, J=7.0 Hz, 2H), 2.67 (t, J=12.1 Hz, 2H), 1.78 (d, J=12.6 Hz, 2H), 1.46 (q, J=6.8 Hz, 2H), 1.36 (s, 1H), 1.21 (d, J=16.8 Hz, 2H).

### Example 69. Synthesis of 4-(2-(2,5-dioxo-2,5-dihydro-1H-pyrrol-1-yl)-7-azaspiro[3.5]nonan-7-yl)phenyl sulfurofluoridate (MFS-10-2)

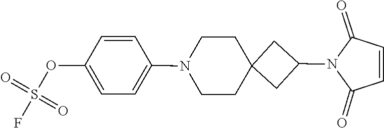 [SMILES: O=C1C=CC(=O)N1C1CC2(CCN(c3ccc(OS(=O)(=O)F)cc3)CC2)C1]

Step 1: To a stirred mixture of tert-butyl N-{7-azaspiro [3.5]nonan-2-yl}carbamate (2 g, 8.321 mmol, 1 equiv) and 1-(benzyloxy)-4-bromobenzene (3.28 g, 12.482 mmol, 1.5 equiv) in toluene (30 mL) were added Pd2(dba)3 (1.52 g, 1.664 mmol, 0.2 equiv), XPhos (0.79 g, 1.664 mmol, 0.2 equiv) and Cs2CO3 (8.13 g, 24.963 mmol, 3 equiv) in portions at room temperature under nitrogen atmosphere. The resulting mixture was stirred at 100° C. for 2 h under nitrogen atmosphere. The resulting mixture was extracted with CH₂Cl₂ (3×30 mL). The combined organic layers were washed with water (3×30 mL), dried over anhydrous Na2SO4. After filtration, the filtrate was concentrated under reduced pressure. The residue was purified by reversed-phase flash chromatography with the following conditions: column, C18 silica gel; mobile phase, MeCN in Water (0.1% FA), 10% to 50% gradient in 10 min; detector, UV 254 nm. This resulted in tert-butyl N-{7-[4-(benzyloxy) phenyl]-7-azaspiro[3.5]nonan-2-yl}carbamate (1.8 g, 51.19% yield) as a brown solid.

Step 2: A mixture of tert-butyl N-{7-[4-(benzyloxy) phenyl]-7-azaspiro [3.5]nonan-2-yl}carbamate (1 g, 2.366 mmol, 1 equiv) in DCM (12 mL) and HCl in 1,4-dioxane (4.0 M) (4 mL). The resulting mixture was stirred at room temperature for 1 h. The precipitated solids were collected by filtration and washed with PE (3×5 mL). This resulted in 7-[4-(benzyloxy) phenyl]-7-azaspiro [3.5]nonan-2-amine (700 mg, 91.73% yield) as a white solid. The crude resulting mixture was used in the next step directly without further purification.

Step 3: To a solution of 7-[4-(benzyloxy) phenyl]-7-azaspiro [3.5]nonan-2-amine (650 mg, 2.016 mmol, 1 equiv) in 10 mL MeOH was added Pd/C (10%, 65 mg) under nitrogen atmosphere in a 40 mL vial. The mixture was hydrogenated at room temperature for 1 h under hydrogen atmosphere using a hydrogen balloon, filtered through a Celite pad and concentrated under reduced pressure. This resulted in 4-{2-amino-7-azaspiro [3.5]nonan-7-yl}phenol (430 mg, 91.82% yield) as a brown solid. The crude resulting mixture was used in the next step directly without further purification.

Step 4: A mixture of 4-{2-amino-7-azaspiro[3.5]nonan-7-yl}phenol (420 mg, 1.808 mmol, 1 equiv), maleic anhydride (265.90 mg, 2.712 mmol, 1.5 equiv) and TEA (548.81 mg, 5.424 mmol, 3 equiv) in ACN (5 mL) was stirred at 90° C. for 2 h. The resulting mixture was concentrated under reduced pressure. The residue was purified by reversed-phase flash chromatography with the following conditions: column, C18 silica gel; mobile phase, MeCN in Water (0.1% FA), 10% to 50% gradient in 10 min; detector, UV 254 nm. This resulted in (2Z)-3-{[7-(4-hydroxyphenyl)-7-azaspiro [3.5]nonan-2-yl]carbamoyl}prop-2-enoic acid (300 mg, 50.23% yield) as a brown oil

Step 5: A mixture of (2Z)-3-{[7-(4-hydroxyphenyl)-7-azaspiro [3.5]nonan-2-yl]carbamoyl}prop-2-enoic acid (300 mg, 0.908 mmol, 1 equiv) in HOAc (5 mL) was stirred at 100° C. for 16 h. The resulting mixture was concentrated under reduced pressure. The residue was purified by reversed-phase flash chromatography with the following conditions: column, C18 silica gel; mobile phase, MeCN in Water (0.1% FA), 10% to 80% gradient in 10 min; detector, UV 254 nm. This resulted in 1-[7-(4-hydroxyphenyl)-7-azaspiro [3.5]nonan-2-yl]pyrrole-2,5-dione (200 mg, 70.51% yield) as a yellow solid.

Step 6: To a stirred mixture of 1-[7-(4-hydroxyphenyl)-7-azaspiro [3.5]nonan-2-yl]pyrrole-2,5-dione (200 mg, 0.640 mmol, 1 equiv) and 3-(fluorosulfonyl)-1,2-dimethylimidazol-1-ium triflate (252.20 mg, 0.768 mmol, 1.2 equiv) in ACN (3 mL) was added TEA (97.19 mg, 0.960 mmol, 1.5 equiv) in portions at −20° C. The resulting mixture was stirred at −20° C. for 30 min. The resulting mixture was extracted with CH₂Cl₂ (3×3 mL). The combined organic layers were washed with water (3×3 mL), dried over anhydrous Na₂SO₄. After filtration, the filtrate was concentrated under reduced pressure. The residue was purified by reversed-phase flash chromatography with the following conditions: column, C18 silica gel; mobile phase, MeCN in Water (0.1% FA), 10% to 95% gradient in 10 min; detector, UV 254 nm. This resulted in 4-[2-(2,5-dioxopyrrol-1-yl)-7-azaspiro [3.5]nonan-7-yl]phenyl sulfurofluoridate (70 mg, 18.48% yield, 99.0% purity) as a yellow solid. LCMS:(ES, m/z): [M+H]⁺=395.1.

¹H NMR (300 MHz, DMSO-d6, ppm) δ 7.36 (dd, J=9.3, 1.0 Hz, 2H), 7.10-7.01 (m, 2H), 6.98 (s, 2H), 4.62-4.43 (m, 1H), 3.18 (dt, J=19.6, 5.6 Hz, 4H), 2.44 (dd, J=12.0, 9.2 Hz, 2H), 2.14 (td, J=9.2, 2.6 Hz, 2H), 1.72 (t, J=5.6 Hz, 4H).

### Example 70. Synthesis of 3-((2-(2,5-dioxo-2,5-dihydro-1H-pyrrol-1-yl)-7-azaspiro[3.5]nonan-7-yl)methyl)phenyl sulfurofluoridate (MFS-10-3)

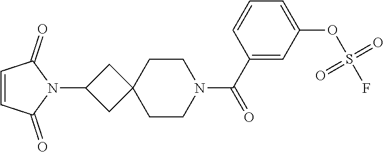 [SMILES: O=C(c1cccc(OS(=O)(=O)F)c1)N1CCC2(CC1)CC(N1C(=O)C=CC1=O)C2]

Step 1: A solution of tert-butyl N-{7-azaspiro[3.5]nonan-2-yl}carbamate (2 g, 8.32 mmol, 1 equiv), 1-(benzyloxy)-3-(bromomethyl)benzene (2.54 g, 9.15 mmol, 1.1 equiv) and TEA (2.53 g, 24.96 mmol, 3 equiv) in MeCN (20 mL) was stirred at room temperature for 4 h under nitrogen atmosphere. The resulting mixture was concentrated under reduced pressure. The residue was purified by silica gel column chromatography, eluted with PE/EA (5:1) to afford tert-butyl N-(7-{[3-(benzyloxy) phenyl]methyl}-7-azaspiro[3.5]nonan-2-yl)carbamate (1.7 g, 46.79% yield) as a yellow oil.

Step 2: A solution of tert-butyl N-(7-{[3-(benzyloxy)phenyl]methyl}-7-azaspiro[3.5]nonan-2-yl)carbamate (1.6 g, 3.665 mmol, 1 equiv) and HCl in 1,4-dioxane (4.0 M) (10 mL) in DCM (10 mL) was stirred at room temperature for 2 h under nitrogen atmosphere. The resulting mixture was concentrated under reduced pressure. This resulted in 7-{[3-(benzyloxy)phenyl]methyl}-7-azaspiro[3.5]nonan-2-amine (1.2 g, Crude Product) as a white solid.

Step 3: A solution of 7-{[3-(benzyloxy) phenyl]methyl}-7-azaspiro[3.5]nonan-2-amine (1.2 g, 3.56 mmol, 1 equiv) and Pd/C (0.19 g, 1.78 mmol, 0.5 equiv) in MeOH (12 mL) was stirred at room temperature for overnight under nitrogen atmosphere. The resulting mixture was filtered, the filter cake was washed with MeOH (3×4 mL). The filtrate was concentrated under reduced pressure. This resulted in 3-({2-amino-7-azaspiro [3.5]nonan-7-yl}methyl)phenol (850 mg, Crude Product) as a yellow oil.

Step 4: A solution of 3-({2-amino-7-azaspiro[3.5]nonan-7-yl}methyl)phenol (850 mg, 3.45 mmol, 1 equiv), maleic anhydride (676.66 mg, 6.90 mmol, 2 equiv) and TEA (1047.44 mg, 10.35 mmol, 3 equiv) in MeCN (10 mL) was stirred at 80° C. for overnight under nitrogen atmosphere. The mixture was allowed to cool down to room temperature. The resulting mixture was concentrated under reduced pressure. The residue was purified by reversed-phase flash chromatography with the following conditions: column, C18 silica gel; mobile phase, MeCN in Water (0.1% FA), 10% to 50% gradient in 10 min; detector, UV 254 nm. This resulted in (2Z)-3-({7-[(3-hydroxyphenyl)methyl]-7-azaspiro[3.5]nonan-2-yl}carbamoyl)prop-2-enoic acid (600 mg, 50.49% yield, 95% purity) as a yellow oil.

Step 5: A solution of (2Z)-3-({7-[(3-hydroxyphenyl) methyl]-7-azaspiro[3.5]nonan-2-yl}carbamoyl)prop-2-enoic acid (600 mg, 1.74 mmol, 1 equiv) and 2,5-dihydrofuran-2,5-dione (512.48 mg, 5.22 mmol, 3 equiv) in HOAc (10 mL) was stirred at 100° C. for overnight under nitrogen atmosphere. The mixture was allowed to cool down to room temperature. The resulting mixture was concentrated under reduced pressure. The residue was purified by reversed-phase flash chromatography with the following conditions: column, C18 silica gel; mobile phase, MeCN in Water (0.1% FA), 10% to 50% gradient in 10 min; detector, UV 254 nm. This resulted in 1-{7-[(3-hydroxyphenyl) methyl]-7-azaspiro[3.5]nonan-2-yl}pyrrole-2,5-dione (440 mg, 77.38% yield, 95% purity) as a yellow oil.

Step 6: A solution of 1-{7-[(3-hydroxyphenyl) methyl]-7-azaspiro [3.5]nonan-2-yl}pyrrole-2,5-dione (440 mg, 1.34 mmol, 1 equiv) in MeCN (5 mL) was treated with 3-(fluorosulfonyl)-1,2-dimethylimidazol-1-ium triflate (885.00 mg, 2.69 mmol, 2 equiv) at 0° C. for 3 min under nitrogen atmosphere followed by the addition of Et₃N (545.66 mg, 5.39 mmol, 4 equiv) dropwise at 0° C. The resulting mixture was stirred at 0° C. for additional 1 h. The resulting mixture was concentrated under reduced pressure. The residue was purified by silica gel column chromatography, eluted with PE/THF (4:1) to afford 3-{[2-(2,5-dioxopyrrol-1-yl)-7-azaspiro[3.5]nonan-7-yl]methyl}phenyl sulfurofluoridate (91.1 mg, 16.55% yield, 96.7% purity) as a light yellow solid. LCMS:(ES, m/z): [M+H]⁺=409.1.

¹H NMR (300 MHz, DMSO-d6, ppm) δ 7.60-7.40 (m, 4H), 6.96 (s, 2H), 4.44 (p, J=8.9 Hz, 1H), 3.50 (s, 2H), 2.44-2.14 (m, 6H), 2.04 (dd, J=11.5, 8.6 Hz, 2H), 1.62 (d, J=6.0 Hz, 4H).

### Example 71. Synthesis of 3-((2-((2,5-dioxo-2,5-dihydro-1H-pyrrol-1-yl)methyl)-7-azaspiro[3.5]nonan-7-yl)methyl)phenyl sulfurofluoridate (MFS-11-2)

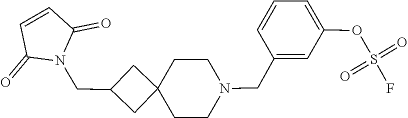 [SMILES: O=C1C=CC(=O)N1CC1CC2(CCN(Cc3cccc(OS(=O)(=O)F)c3)CC2)C1]

Step 1: To a stirred mixture of M-cresol (2 g, 18.495 mmol, 1.00 equiv) and 3-(fluorosulfonyl)-1,2-dimethylimidazol-1-ium triflate (6.68 g, 20.345 mmol, 1.1 equiv) in ACN (35 mL) was added TEA (3.74 g, 36.990 mmol, 2 equiv) dropwise at 0° C. The resulting mixture was stirred at room temperature for additional 1 h. The reaction was monitored by TLC. The resulting mixture was concentrated under reduced pressure. The residue was purified by silica gel column chromatography, eluted with PE/EA (5:1) to afford 3-methylphenyl sulfurofluoridate (2.5 g, 71.07% yield) as a white solid.

Step 2: To a stirred mixture of 3-methylphenyl sulfurofluoridate (2.5 g, 13.145 mmol, 1.00 equiv) and NBS (2.34 g, 13.145 mmol, 1.00 equiv) in CCl₄ (50 mL) was added BPO (0.32 g, 1.315 mmol, 0.1 equiv) in portions at room temperature. The resulting mixture was stirred at 80° C. for additional 3 h. The reaction was monitored by LCMS. The resulting mixture was concentrated under reduced pressure. The residue was purified by reversed-phase flash chromatography with the following conditions: column, C18 silica gel; mobile phase, MeCN in Water (0.1% FA), 10% to 70% gradient in 10 min; detector, UV 254 nm. This resulted in 3-(bromomethyl) phenyl sulfurofluoridate (1.1 g, 31.10% yield) as a colorless oil.

Step 3: A solution of 1-{7-azaspiro [3.5]nonan-2-ylmethyl}pyrrole-2,5-dione (200 mg, 0.854 mmol, 1.00 equiv) in ACN (8 mL) was treated with TEA (129.57 mg, 1.281 mmol, 1.5 equiv) at 0° C. for 10 min followed by the addition of 3-(bromomethyl)phenyl sulfurofluoridate (275.63 mg, 1.025 mmol, 1.2 equiv) in portions at room temperature. The resulting mixture was stirred at room temperature for additional 1 h. The reaction was monitored by LCMS. The residue was purified by reversed-phase flash chromatography with the following conditions: column, C18 silica gel; mobile phase, MeCN in Water (0.1% FA), 10% to 60% gradient in 10 min; detector, UV 254 nm. This resulted in 3-({2-[(2,5-dioxopyrrol-1-yl) methyl]-7-azaspiro [3.5]nonan-7-yl}methyl) phenyl sulfurofluoridate (6 mg, 1.66% yield, 98.6% purity) as a light yellow oil. LCMS: (ES, m/z): [M+H]⁺=423.1.

¹H NMR (300 MHz, DMSO-d₆, ppm) δ 7.58-7.40 (m, 4H), 7.01 (s, 2H), 3.48 (s, 2H), 3.37 (d, J=23.7 Hz, 2H), 2.48-2.37 (m, 1H), 2.24 (d, J=18.5 Hz, 4H), 1.78 (td, J=8.9, 2.4 Hz, 2H), 1.58-1.39 (m, 6H).

### Example 72. Synthesis of 4-(2-(2,5-dioxo-2,5-dihydro-1H-pyrrol-1-yl)-7-azaspiro[3.5]nonane-7-carbonyl)phenyl sulfurofluoridate (MFS-11-5)

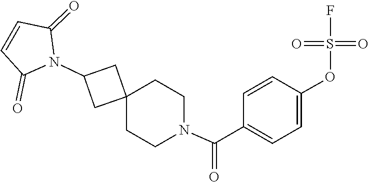 [SMILES: O=C(c1ccc(OS(=O)(=O)F)cc1)N1CCC2(CC1)CC(N1C(=O)C=CC1=O)C2]

To a stirred mixture of 4-[(fluorosulfonyl) oxy]benzoic acid (359.83 mg, 1.634 mmol, 1.2 equiv), HATU (1035.72 mg, 2.724 mmol, 2 equiv) and DIEA (528.08 mg, 4.086 mmol, 3 equiv) in DCM (5 mL) at room temperature under nitrogen atmosphere. The mixture was stirred for 15 min. 1-{7-azaspiro [3.5]nonan-2-yl}pyrrole-2,5-dione (300 mg, 1.362 mmol, 1 equiv) was added and stirred for 30 min. The resulting mixture was extracted with CH₂Cl₂ (3×5 mL). The combined organic layers were washed with water (3×5 mL), dried over anhydrous Na₂SO₄. After filtration, the filtrate was concentrated under reduced pressure. The residue was purified by reversed-phase flash chromatography with the following conditions: column, C18 silica gel; mobile phase, MeCN in Water (0.1% FA), 10% to 95% gradient in 10 min; detector, UV 254 nm. This resulted in 4-[2-(2,5-dioxopyrrol-1-yl)-7-azaspiro[3.5]nonane-7-carbonyl]phenyl sulfurofluoridate (200 mg, 34.76% yield, 100.0% purity) as a white solid. LCMS:(ES, m/z): [M+H]⁺=423.0 ¹H NMR (400 MHz, DMSO-d₆, ppm) δ 7.68 (d, J=8.5 Hz, 2H), 7.60 (d, J=8.5 Hz, 2H), 6.97 (s, 2H), 4.48 (s, 1H), 3.57 (d, J=26.1 Hz, 2H), 3.20 (d, J=20.4 Hz, 2H), 2.44 (s, 2H), 2.15 (s, 2H), 1.65 (d, J=32.8 Hz, 4H).

### Example 73. Synthesis of 4-(2-(2,5-dioxo-2,5-dihydro-1H-pyrrol-1-yl)-7-azaspiro[3.5]nonane-7-carbonyl)-2-fluorophenyl sulfurofluoridate (MFS-11-6)

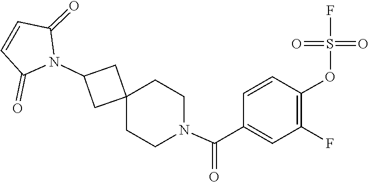 [SMILES: O=C(c1ccc(OS(=O)(=O)F)c(F)c1)N1CCC2(CC1)CC(N1C(=O)C=CC1=O)C2]

Step 1: To a stirred mixture of 3-fluoro-4-hydroxybenzoic acid (1 g, 6.406 mmol, 1 equiv) and (4-acetamidophenyl) (fluorosulfonyl)aminosulfonyl fluoride (3.02 g, 9.609 mmol, 1.5 equiv) in THF (15 mL) were added DBU (1.95 g, 12.812 mmol, 2 equiv) in portions at 0° C. The resulting mixture was stirred at room temperature for 1 h. The mixture was acidified neutralized to pH 3 with HCl (3M). The resulting mixture was extracted with CH₂Cl₂ (3×15 mL). The combined organic layers were washed with water (3×15 mL), dried over anhydrous Na₂SO₄. After filtration, the filtrate was concentrated under reduced pressure. The residue was purified by reversed-phase flash chromatography with the following conditions: column, C18 silica gel; mobile phase, MeCN in Water (0.1% FA), 10% to 95% gradient in 10 min; detector, UV 254 nm. This resulted in 3-fluoro-4-[(fluorosulfonyl) oxy]benzoic acid (800 mg, 52.44% yield) as a white solid.

Step 2: A mixture of 3-fluoro-4-[(fluorosulfonyl) oxy]benzoic acid (389.24 mg, 1.634 mmol, 1.2 equiv), DIEA (528.08 mg, 4.086 mmol, 3 equiv) and HATU (776.79 mg, 2.043 mmol, 1.5 equiv) in DCM (10 mL) was stirred at room temperature for 30 min. To the above mixture was added 1-{7-azaspiro[3.5]nonan-2-yl}pyrrole-2,5-dione (300 mg, 1.362 mmol, 1 equiv) in portions at room temperature. The resulting mixture was stirred at room temperature for 1 h. The resulting mixture was extracted with CH₂Cl₂ (3×10 mL). The combined organic layers were washed with water (3×10 mL), dried over anhydrous Na₂SO₄. After filtration, the filtrate was concentrated under reduced pressure. The residue was purified by reversed-phase flash chromatography with the following conditions: column, C18 silica gel; mobile phase, MeCN in Water (0.1% FA), 10% to 95% gradient in 10 min; detector, UV 254 nm. This resulted in 4-[2-(2,5-dioxopyrrol-1-yl)-7-azaspiro [3.5]nonane-7-carbonyl]-2-fluorophenyl sulfurofluoridate (200 mg, 33.34% yield, 99.6% purity) as a white solid. LCMS:ES, m/z): [M+H]⁺=441.0 ¹H NMR (400 MHz, DMSO-d₆, ppm) δ 7.90 (t, J=8.0 Hz, 1H), 7.70 (d, J=10.3 Hz, 1H), 7.42 (d, J=8.4 Hz, 1H), 6.97 (s, 2H), 4.49 (d, J=9.7 Hz, 1H), 3.56 (d, J=25.8 Hz, 2H), 3.20 (d, J=20.9 Hz, 2H), 2.44 (d, J=11.0 Hz, 2H), 2.15 (d, J=10.0 Hz, 2H), 1.65 (d, J=31.8 Hz, 4H).

### Example 74. Synthesis of 4-((2-((2,5-dioxo-2,5-dihydro-1H-pyrrol-1-yl)methyl)-7-azaspiro[3.5]nonan-7-yl)methyl)phenyl sulfurofluoridate (MFS-12-1)

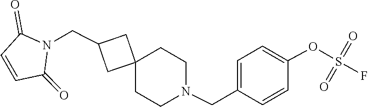 [SMILES: O=C1C=CC(=O)N1CC1CC2(CCN(Cc3ccc(OS(=O)(=O)F)cc3)CC2)C1]

Step 1: To a stirred mixture of P-cresol (2 g, 18.495 mmol, 1.00 equiv) and 3-(fluorosulfonyl)-1,2-dimethylimidazol-1-ium triflate (6.68 g, 20.345 mmol, 1.1 equiv) in ACN (35 mL) was added TEA (3.74 g, 36.990 mmol, 2 equiv) dropwise at 0° C. The resulting mixture was stirred at room temperature for additional 1 h. The reaction was monitored by TLC. The resulting mixture was concentrated under reduced pressure. The residue was purified by silica gel column chromatography, eluted with PE/EA (5:1) to afford 4-methylphenyl sulfurofluoridate (2.5 g, 71.07% yield) as a white solid.

Step 2: To a stirred mixture of 4-methylphenyl sulfurofluoridate (2.5 g, 13.145 mmol, 1.00 equiv) and NBS (2.34 g, 13.145 mmol, 1.00 equiv) in CCl₄ (50 mL) was added BPO (0.32 g, 1.315 mmol, 0.1 equiv) in portions at room temperature. The resulting mixture was stirred at 80° C. for additional 3 h. The reaction was monitored by LCMS. The resulting mixture was concentrated under reduced pressure. The residue was purified by reversed-phase flash chromatography with the following conditions: column, C18 silica gel; mobile phase, MeCN in Water (0.1% FA), 10% to 70% gradient in 10 min; detector, UV 254 nm. This resulted in 4-(bromomethyl) phenyl sulfurofluoridate (1.1 g, 31.10% yield) as a colorless oil.

Step 3: A solution of 1-{7-azaspiro [3.5]nonan-2-ylmethyl}pyrrole-2,5-dione (200 mg, 0.854 mmol, 1.00 equiv) in ACN (8 mL) was treated with TEA (129.57 mg, 1.281 mmol, 1.5 equiv) at 0° C. for 10 min followed by the addition of 4-(bromomethyl)phenyl sulfurofluoridate (275.63 mg, 1.025 mmol, 1.2 equiv) in portions at room temperature. The resulting mixture was stirred at room temperature for additional 1 h. The reaction was monitored by LCMS. The residue was purified by reversed-phase flash chromatography with the following conditions: column, C18 silica gel; mobile phase, MeCN in Water (0.1% FA), 10% to 60% gradient in 10 min; detector, UV 254 nm. This resulted in 4-({2-[(2,5-dioxopyrrol-1-yl) methyl]-7-azaspiro [3.5]nonan-7-yl}methyl) phenyl sulfurofluoridate (12 mg, 3.33% yield, 99.1% purity) as a light yellow oil. LCMS: (ES, m/z): [M+H]⁺=423.0.

¹H NMR (300 MHz, DMSO-d₆, ppm) δ 7.52 (s, 4H), 7.02 (s, 2H), 3.43 (d, J=7.2 Hz, 4H), 2.43 (q, J=7.8 Hz, 1H), 2.34-2.13 (m, 3H).1.78 (s, 2H), 1.62-1.34 (m, 7H).

### Example 75. Synthesis of 5-((2,5-dioxo-2,5-dihydro-1H-pyrrol-1-yl)methyl)-2-methoxybenzenesulfonyl fluoride (SFY-4-1)

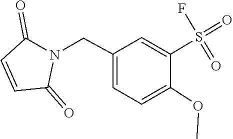 [SMILES: COc1ccc(CN2C(=O)C=CC2=O)cc1S(=O)(=O)F]

Step 1: To a stirred solution of 5-bromo-2-methoxybenzenesulfonyl chloride (5 g, 17.511 mmol, 1 equiv) and 18-crown-6 (0.46 g, 1.751 mmol, 0.1 equiv) in ACN (20 mL) was added KF (3.05 g, 52.533 mmol, 3 equiv) dropwise at room temperature. The resulting mixture was stirred at room temperature for 18 h. The resulting mixture was filtered, the filter cake was washed with ACN (3×3 mL). The filtrate was concentrated under reduced pressure. The residue was purified by reversed-phase flash chromatography with the following conditions: column, C18 silica gel; mobile phase, MeCN in Water (0.1% FA), 10% to 50% gradient in 10 min; detector, UV 254 nm. This resulted in 5-bromo-2-methoxybenzenesulfonyl fluoride (3.2 g, 67.91% yield) as a white solid.

Step 2: To a stirred solution of 5-bromo-2-methoxybenzenesulfonyl fluoride (1 g, 3.716 mmol, 1 equiv), potassium tert-butyl N-[(trifluoroboranuidyl)methyl]carbamate (1.32 g, 5.574 mmol, 1.5 equiv) and Cs₂CO₃ (3.63 g, 11.148 mmol, 3 equiv) in Dioxane (10 mL) and H₂O (2 mL) were added Catacxium A (0.27 g, 0.743 mmol, 0.2 equiv) and Pd(OAc)₂ (0.17 g, 0.743 mmol, 0.2 equiv) dropwise at room temperature under nitrogen atmosphere. The resulting mixture was stirred at 100° C. for 1 h under nitrogen atmosphere. The resulting mixture was filtered, the filter cake was washed with 1,4-dioxane (3×2 mL). The filtrate was concentrated under reduced pressure. The residue was purified by reversed-phase flash chromatography with the following conditions: column, C18 silica gel; mobile phase, MeCN in Water (0.1% FA), 10% to 50% gradient in 10 min; detector, UV 254 nm. This resulted in tert-butyl N-{[3-(fluorosulfonyl)-4-methoxyphenyl]methyl}carbamate (400 mg, 33.70% yield) as a white solid.

Step 3: To a stirred solution of tert-butyl N-{[3-(fluorosulfonyl)-4-methoxyphenyl]methyl}carbamate (400 mg, 1.253 mmol, 1 equiv) in DCM (3 mL) and HCl in 1,4-dioxane (3 mL) at room temperature under nitrogen atmosphere. The resulting mixture was stirred at room temperature for additional 1 h. The resulting mixture was filtered, the filter cake was washed with DCM (3×3 mL). The filtrate was concentrated under reduced pressure. This resulted in 5-(aminomethyl)-2-methoxybenzenesulfonyl fluoride hydrochloride (200 mg, 62.45% yield) as a light yellow solid.

Step 4: To a stirred solution of 5-(aminomethyl)-2-methoxybenzenesulfonyl fluoride hydrochloride (200 mg, 0.782 mmol, 1 equiv) in NaHCO₃(aq.) (1 mL) and 1,4-dioxane (3 mL) was added methyl 2,5-dioxopyrrole-1-carboxylate (181.99 mg, 1.173 mmol, 1.5 equiv) in portions at 0° C. under nitrogen atmosphere. The resulting mixture was stirred at room temperature for additional overnight. The resulting mixture was diluted with 20 mL H₂O and extracted with EtOAc (10 mL×3). The combined organic layers were washed with brine (10 mL), dried over anhydrous Na₂SO₄. After filtration, the filtrate was concentrated under reduced pressure. This resulted in 5-[(2,5-dioxopyrrol-1-yl) methyl]-2-methoxybenzenesulfonyl fluoride (39.1 mg, 16.70% yield, 98.9% purity) as a light yellow oil. GCMS:(ES, m/z): [M]⁺=299.0.

¹H NMR (300 MHz, DMSO-d₆, ppm) δ 7.83 (d, J=2.3 Hz, 1H), 7.75 (dd, J=8.7, 2.4 Hz, 1H), 7.41 (d, J=8.8 Hz, 1H), 7.08 (s, 2H), 4.64 (s, 2H), 3.98 (s, 3H).

### Example 76. Synthesis of 4-(2,5-dioxo-2,5-dihydro-1H-pyrrol-1-yl)benzenesulfonyl fluoride (MSF-4-1)

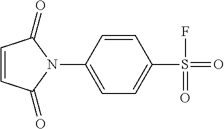 [SMILES: O=C1C=CC(=O)N1c1ccc(S(=O)(=O)F)cc1]

Step 1: To a stirred solution of 4-nitrobenzenesulfonyl fluoride (2 g, 9.74 mmol, 1 equiv) in MeOH (20 ml) was added Pd/C (10% purity, 200 mg), the mixture was degassed and purged with hydrogen for several times, then stirred at 60° C. for 12 hours. The mixture was filtered, the filtrate was concentrated under reduced pressure in vacuum to afford 4-aminobenzenesulfonyl fluoride (1.5 g, 87.7% yield). as a yellow solid.

Step 2: To a stirred solution of 4-aminobenzenesulfonyl fluoride (1.5 g, 7.84 mmol, 1 equiv) and maleic anhydride (0.92 g, 9.41 mmol, 1.2 equiv) in HOAc (20 mL) at room temperature under nitrogen atmosphere. The resulting mixture was stirred at 100° C. for 16 h. The resulting mixture was concentrated under reduced pressure. The residue was purified by silica gel column chromatography, eluted with PE: EA (5:1) to afford 4-(2,5-dioxo-2,5-dihydro-1H-pyrrol-1-yl) benzenesulfonyl fluoride (420 mg, 19.26% yield, 97.5% purity) as a light white solid. GCMS:(ES, m/z): [M+H]⁺=255.0.

¹H NMR (300 MHz, DMSO-d₆) δ 8.47-8.22 (m, 2H), 7.84 (d, J=8.7 Hz, 2H), 7.29 (s, 2H).

### Example 77. Synthesis of 3-(2-(2,5-dioxo-2,5-dihydro-1H-pyrrol-1-yl)ethyl)benzenesulfonyl fluoride (MSF-5-1)

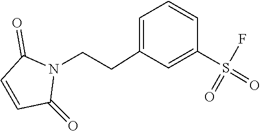 [SMILES: O=C1C=CC(=O)N1CCc1cccc(S(=O)(=O)F)c1]

Step 1: To a stirred solution of potassium 3-bromobenzenesulfonylfluoride tert-butyl N-[2-(trifluoroboranuidyl) ethyl]carbamate (1.5 g, 3.06 mmol, 1 equiv) and potassium tert-butyl N-[2-(trifluoroboranuidyl) ethyl]carbamate (0.92 g, 3.67 mmol, 1.2 equiv), Pd(OAc)2 (0.14 g, 0.61 mmol, 0.2 equiv), ruPhos (0.14 g, 0.30 mmol, 0.1 equiv) and Cs2CO3 (2.99 g, 9.18 mmol, 3 equiv) in toluene (20 mL) and H2O (5 mL) at room temperature under nitrogen atmosphere. The resulting mixture was stirred at 100° C. for additional 3 h. The mixture was allowed to cool down to room temperature. The resulting mixture was diluted with 25 mL H2O and extracted with EtOAc (25 mL×3). The combined organic layers were washed with brine (25 mL), dried over anhydrous Na2SO4. After filtration, the filtrate was concentrated under reduced pressure. The residue was purified by silica gel column chromatography, eluted with PE/THF (3/1) to afford tert-butyl N-{2-[4-(fluorosulfonyl) phenyl]ethyl}carbamate (850 mg, 44.66% yield) as a yellow solid.

Step 2: To a stirred tert-butyl N-{2-[4-(fluorosulfonyl) phenyl]ethyl}carbamate (850 mg, 2.80 mmol, 1 equiv) in DCM (4 mL) and HCl in 1,4-dioxane (4.0 M) (4 mL) at room temperature under nitrogen atmosphere. The resulting mixture was stirred at room temperature for additional 1 h. The resulting mixture was concentrated under reduced pressure to afford 3-(2-aminoethyl) benzene sulfonyl fluoride (500 mg, 87.80% yield) as a light yellow solid. The crude product was used in the next step directly without further purification.

Step 3: To a stirred tert-butyl N-{2-[4-(fluorosulfonyl) phenyl]ethyl}carbamate (850 mg, 2.80 mmol, 1 equiv) in DCM (4 mL) and HCl in 1,4-dioxane (4.0 M) (4 mL) at room temperature under nitrogen atmosphere. The resulting mixture was stirred at room temperature for additional 1 h. The resulting mixture was concentrated under reduced pressure to afford 3-(2-aminoethyl) benzene sulfonyl fluoride (500 mg, 87.80% yield) as a light yellow solid. The crude product was used in the next step directly without further purification.

Step 4: To a stirred solution of 3-(2-aminoethyl) benzenesulfonyl fluoride (500 mg, 2.46 mmol, 1 equiv) and maleic anhydride (723.74 mg, 7.38 mmol, 3 equiv) in MeCN (5 mL) were added TEA (1244.81 mg, 12.30 mmol, 5 equiv) dropwise at 0° C. under nitrogen atmosphere. The resulting mixture was stirred at room temperature for additional 2 h. The resulting mixture was concentrated under vacuum. The residue was purified by silica gel column chromatography, eluted with CH₂Cl₂/MeOH (4/1) to afford (2Z)-3-({2-[3-(fluorosulfonyl) phenyl]ethyl}carbamoyl) prop-2-enoic acid (320 mg, 43.17% yield) as a light yellow oil.

Step 5: To a stirred solution of (2Z)-3-({2-[3-(fluorosulfonyl) phenyl]ethyl}carbamoyl) prop-2-enoic acid (320 mg, 1.06 mmol, 1 equiv) and maleic anhydride (312.44 mg, 3.18 mmol, 3 equiv) in HOAc (4 mL) at room temperature under nitrogen atmosphere. The resulting mixture was stirred at 100° C. for 16 h. The resulting mixture was concentrated under reduced pressure. The residue was purified by reversed-phase flash chromatography with the following conditions: column, C18 silica gel; mobile phase, MeCN in Water (0.1% FA), 10% to 50% gradient in 10 min; detector, UV 254 nm. The resulted in 3-{4-[(2,5-dioxopyrrol-1-yl)methyl]piperidin-1-yl}benzene sulfonyl fluoride (160 mg, 53.18% yield, 98.6% purity) as a light yellow solid. GCMS: (EI+, m/z, M+)=283.0.

¹H NMR (300 MHz, DMSO-d₆) δ 8.01-7.91 (m, 2H), 7.80-7.63 (m, 2H), 6.97 (s, 2H), 3.72 (t, J=6.7 Hz, 2H), 3.01 (t, J=6.7 Hz, 2H).

### Example 78. Synthesis of (S)-1-(2,5-dioxo-2,5-dihydro-1H-pyrrol-1-yl)-2,3-dihydro-1H-inden-5-yl sulfurofluoridate (MFS-5-11)

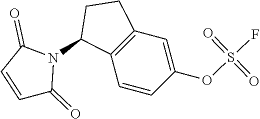 [SMILES: O=C1C=CC(=O)N1[C@H]1CCc2cc(OS(=O)(=O)F)ccc21]

Step 1: A solution of (1S)-1-amino-2,3-dihydro-1H-inden-5-ol (300 mg, 2.011 mmol, 1 equiv) in 1,4-dioxane (12 mL) and 50% NaHCO₃ (3 mL) was treated with methyl 2,5-dioxopyrrole-1-carboxylate (311.90 mg, 2.011 mmol, 1 equiv) at 0° C. The resulting mixture was stirred at 20° C. for 2 h. The reaction was monitored by LCMS. The resulting mixture was extracted with EtOAc (3×2 mL). The combined organic layers were washed with water (3×1 mL), dried over anhydrous Na₂SO₄. After filtration, the filtrate was concentrated under reduced pressure. The residue was purified by silica gel column chromatography, eluted with PE/EA (5:1) to afford 1-[(1S)-5-hydroxy-2,3-dihydro-1H-inden-1-yl]pyrrole-2,5-dione (150 mg, 32.54% yield) as a white solid.

Step 2: To a stirred solution/mixture of 3-(fluorosulfonyl)-1,2-dimethylimidazol-1-ium triflate (214.79 mg, 0.65 mmol, 1.5 equiv) and 1-[(1S)-5-hydroxy-2,3-dihydro-1H-inden-1-yl]pyrrole-2,5-dione (100 mg, 0.43 mmol, 1 equiv) in DCM was added TEA (88.29 mg, 0.87 mmol, 2 equiv) dropwise at 0° C. under nitrogen atmosphere. The reaction was monitored by LCMS. The residue was purified by silica gel column chromatography, eluted with PE/EA (1:1) to afford (1S)-1-(2,5-dioxopyrrol-1-yl)-2,3-dihydro-1H-inden-5-yl sulfurofluoridate (55 mg, 40.50% yield, 99.8% purity) as a white solid. GCMS: (ES, m/z): [M]⁺=311.0.

¹H NMR (300 MHz, DMSO-d₆, ppm) δ 7.51 (d, J=2.0 Hz, 1H), 7.39-7.25 (m, 2H), 7.04 (s, 2H), 5.58 (t, J=8.3 Hz, 1H), 3.14 (ddd, J=13.2, 9.4, 4.8 Hz, 1H), 2.96 (dt, J=16.6, 8.2 Hz, 1H), 2.43 (td, J=8.6, 4.0 Hz, 6H), 2.36-2.23 (m, 1H).

### Example 79. Synthesis of 4-(2-(2,5-dioxo-2,5-dihydro-1H-pyrrol-1-yl)ethyl)benzenesulfonyl fluoride (MSF-6-1)

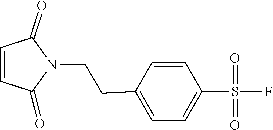 [SMILES: O=C1C=CC(=O)N1CCc1ccc(S(=O)(=O)F)cc1]

Step 1: To a stirred solution of 4-bromobenzenesulfonyl fluoride (3 g, 12.549 mmol, 1 equiv) and potassium tert-butyl N-[2-(trifluoroboranuidyl) ethyl]carbamate (3.78 g, 15.05 mmol, 1.2 equiv), Pd(OAc)₂ (0.28 g, 1.25 mmol, 0.1 equiv), RuPhos (1.17 g, 2.51 mmol, 0.2 equiv) and Cs₂CO₃ (5.98 g, 18.36 mmol, 3 equiv) in toluene (40 mL) and H₂O (5 mL) at room temperature under nitrogen atmosphere. The resulting mixture was stirred at 100° C. for additional 3 h. The mixture was allowed to cool down to room temperature. The resulting mixture was diluted with 50 mL H₂O and extracted with EtOAc (50 mL×3). The combined organic layers were washed with brine (50 mL) dried over anhydrous Na₂SO₄. After filtration, the filtrate was concentrated under reduced pressure. The residue was purified by silica gel column chromatography, eluted with PE/THF (3/1) to afford tert-butyl (4-(fluorosulfonyl) phenethyl) carbamate (1.60 g, 42.03% yield) as a yellow solid.

Step 2: To a stirred solution of tert-butyl (4-(fluorosulfonyl) phenethyl) carbamate (1.6 g, 5.27 mmol, 1 equiv) in DCM (8 mL) and HCl in 1,4-dioxane (4.0 M) (8 mL) at room temperature under nitrogen atmosphere. The resulting mixture was stirred at room temperature for additional 1 h. The resulting mixture was concentrated under reduced pressure to afford 4-(2-aminoethyl) benzene esulfonyl fluoride (1.2 g, 111.95% yield) as a light yellow solid. The crude product was used in the next step directly without further purification.

Step 3: To a stirred solution of 4-(2-aminoethyl) benzene esulfonyl fluoride (1.2 g, 5.905 mmol, 1 equiv) and maleic anhydride (1.74 g, 17.71 mmol, 3 equiv) in MeCN (20 mL) were added TEA (2.99 g, 29.52 mmol, 5 equiv) dropwise at 0° C. under nitrogen atmosphere. The resulting mixture was stirred at room temperature for additional 2 h. The resulting mixture was concentrated under vacuum. The residue was purified by silica gel column chromatography, eluted with CH₂Cl₂/MeOH (4/1) to afford (2Z)-3-({2-[4-(fluorosulfonyl) phenyl]ethyl}carbamoyl) prop-2-enoic acid (800 mg, 44.97% yield) as a light yellow oil.

Step 3: To a stirred solution of (2Z)-3-({2-[4-(fluorosulfonyl) phenyl]ethyl}carbamoyl) prop-2-enoic acid (800 mg, 2.65 mmol, 1 equiv) and maleic anhydride (781.10 mg, 7.96 mmol, 3 equiv) in HOAc (10 mL) at room temperature under nitrogen atmosphere. The resulting mixture was stirred at 100° C. for 16 h. The resulting mixture was concentrated under reduced pressure. The residue was purified by reversed-phase flash chromatography with the following conditions: column, C18 silica gel; mobile phase, MeCN in Water (0.1% FA), 10% to 50% gradient in 10 min; detector, UV 254 nm. The resulted in 3-{4-[(2,5-dioxopyrrol-1-yl) methyl]piperidin-1-yl}benzene sulfony fluoride (380 mg, 50.52% yield, 99.4% purity) as a light yellow solid. GCMS: GC-MS: (EI+, m/z, M⁺)=283.0.

¹H NMR (300 MHz, DMSO-d₆) δ 8.08-7.99 (m, 2H), 7.60 (d, J=8.1 Hz, 2H), 6.99 (s, 2H), 3.72 (t, J=6.9 Hz, 2H), 3.00 (t, J=6.8 Hz, 2H).

### Example 80. Synthesis of 3-(4-(2,5-dioxo-2,5-dihydro-1H-pyrrol-1-yl)piperidin-1-yl)benzenesulfonyl fluoride (MSF-7-1)

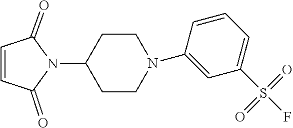 [SMILES: O=C1C=CC(=O)N1C1CCN(c2cccc(S(=O)(=O)F)c2)CC1]

Step 1: Into a 40 mL sealed tube were added 3-bromobenzenesulfonyl fluoride (2 g, 8.36 mmol, 1 equiv), tert-butyl N-(piperidin-4-yl)carbamate (1.84 g, 9.20 mmol, 1.1 equiv), Dioxane (20 mL), Cs2CO3 (5.45 g, 16.73 mmol, 2 equiv) and RuPhos Pd G3 (0.70 g, 0.83 mmol, 0.1 equiv) at room temperature. The resulting mixture was stirred at 100° C. for overnight under nitrogen atmosphere. The mixture was allowed to cool down to room temperature. The reaction was quenched by the addition of water (50 mL) at room temperature. The resulting mixture was extracted with EtOAc (2×50 mL). The combined organic layers were washed with brine (1×50 mL), dried over anhydrous Na2SO4. After filtration, the filtrate was concentrated under reduced pressure. The residue was purified by reversed-phase flash chromatography with the following conditions: column, C18 silica gel; mobile phase, MeCN in Water (0.1% NH3·H2O), 5% to 95% gradient in 15 min; detector, UV 254 nm. This resulted in tert-butyl N-{1-[3-(fluorosulfonyl)phenyl]piperidin-4-yl}carbamate (1.2 g, 40.02% yield, 95% purity) as a yellow oil.

Step 2: Into a 40 mL sealed tube were added tert-butyl N-{1-[3-(fluorosulfonyl)phenyl]piperidin-4-yl}carbamate (1.2 g, 3.348 mmol, 1 equiv), DCM (5 mL) and HCl in 1,4-dioxane (4.0 M) (5 mL) at room temperature. The resulting mixture was stirred at room temperature for 2 h under nitrogen atmosphere. The resulting mixture was concentrated under reduced pressure. The crude product was used in the next step directly without further purification.

Step 3: Into a 40 mL sealed tube were added 3-(4-aminopiperidin-1-yl)benzenesulfonyl fluoride (700 mg, 2.71 mmol, 1 equiv), MeCN (10 mL) and maleic anhydride (797.18 mg, 8.13 mmol, 3 equiv) at room temperature. To the above mixture was added Et3N (822.68 mg, 8.13 mmol, 3 equiv) dropwise over 3 min at 0° C. The resulting mixture was stirred at room temperature for additional 1 h. The residue was purified by reversed-phase flash chromatography with the following conditions: column, C18 silica gel; mobile phase, MeCN in Water (0.1% FA), 10% to 50% gradient in 10 min; detector, UV 254 nm. This resulted in (2Z)-3-({1-[3-(fluorosulfonyl)phenyl]piperidin-4-yl}carbamoyl)prop-2-enoic acid (600 mg, 62.13% yield, 95% purity) as a yellow oil.

Step 4: To a stirred solution of 3-[4-(aminomethyl)piperidin-1-yl]benzenesulfonyl fluoride (400 mg, 1.46 mmol, 1 equiv) and maleic anhydride (432.06 mg, 4.40 mmol, 3 equiv) in HOAc (4 mL) at room temperature under nitrogen atmosphere. The resulting mixture was stirred at 100° C. for additional overnight. The mixture was allowed to cool down to room temperature. The resulting mixture was washed with 3×4 mL of water. The aqueous layer was extracted with EtOAc (3×4 mL). The combined organic layers were dried over anhydrous Na2SO4. After filtration, the filtrate was concentrated under reduced pressure. The residue was purified by reversed-phase flash chromatography with the following conditions: column, C18 silica gel; mobile phase, MeCN in Water (0.1% TFA), 10% to 50% gradient in 10 min; detector, UV 254 nm. This resulted in 3-{4-[(2,5-dioxopyrrol-1-yl)methyl]piperidin-1-yl}benzenesulfonyl fluoride (60 mg, 11.59% yield, 97.9% purity) as a light yellow oil. LCMS:(ES, m/z): [M+H]⁺=339.2.

¹H NMR (300 MHz, DMSO-d₆) δ 7.63-7.49 (m, 2H), 7.47 (t, J=2.2 Hz, 1H), 7.41 (d, J=7.4 Hz, 1H), 6.99 (s, 2H), 4.08 (ddt, J=12.2, 8.1, 4.1 Hz, 1H), 3.98 (d, J=13.0 Hz, 2H), 2.92 (td, J=12.8, 2.4 Hz, 2H), 2.22 (qd, J=12.5, 4.0 Hz, 2H), 1.71 (d, J=12.6 Hz, 2H).

### Example 81. Synthesis of 3-(4-((2,5-dioxo-2,5-dihydro-1H-pyrrol-1-yl)methyl)piperidin-1-yl)benzenesulfonyl fluoride (MSF-8-1)

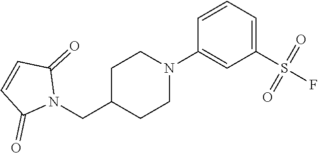 [SMILES: O=C1C=CC(=O)N1CC1CCN(c2cccc(S(=O)(=O)F)c2)CC1]

Step 1: Into a 40 mL sealed tube were added 3-bromobenzenesulfonyl fluoride (1.3 g, 5.43 mmol, 1 equiv), tert-butyl N-(piperidin-4-ylmethyl)carbamate (1.28 g, 5.98 mmol, 1.1 equiv), Dioxane (15 mL), Cs2CO3 (3.54 g, 10.87 mmol, 2 equiv) and RuPhos Pd G3 (0.45 g, 0.54 mmol, 0.1 equiv) at room temperature. The resulting mixture was stirred at 100° C. for overnight under nitrogen atmosphere. The mixture was allowed to cool down to room temperature. The reaction was quenched by the addition of water (50 mL) at room temperature. The resulting mixture was extracted with EtOAc (2×50 mL). The combined organic layers were washed with brine (1×50 mL), dried over anhydrous Na2SO4. After filtration, the filtrate was concentrated under reduced pressure. The residue was purified by reversed-phase flash chromatography with the following conditions: column, C18 silica gel; mobile phase, MeCN in Water (0.1% NH3·H2O), 10% to 50% gradient in 10 min; detector, UV 254 nm. This resulted in tert-butyl N-({1-[3-(fluorosulfonyl)phenyl]piperidin-4-yl}methyl)carbamate (700 mg, 34.56% yield, 95% purity) as a light yellow solid.

Step 2: Into a 40 mL sealed tube were added tert-butyl N-({1-[3-(fluorosulfonyl)phenyl]piperidin-4-yl}methyl)carbamate (700 mg, 1.87 mmol, 1 equiv), DCM (5 mL) and HCl in 1,4-dioxane (4.0 M) (5 mL) at room temperature. The resulting mixture was stirred at room temperature for 1 h under nitrogen atmosphere. The resulting mixture was concentrated under reduced pressure. The crude product was used in the next step directly without further purification.

Step 3: To a stirred solution of 3-[4-(aminomethyl)piperidin-1-yl]benzenesulfonyl fluoride (400 mg, 1.469 mmol, 1 equiv) and maleic anhydride (432.06 mg, 4.407 mmol, 3 equiv) in HOAc (4 mL) at room temperature under nitrogen atmosphere. The resulting mixture was stirred at 100° C. for additional overnight. The mixture was allowed to cool down to room temperature. The resulting mixture was washed with 3×4 mL of water. The aqueous layer was extracted with EtOAc (3×4 mL). The combined organic layers were dried over anhydrous Na2SO4. After filtration, the filtrate was concentrated under reduced pressure. The residue was purified by reversed-phase flash chromatography with the following conditions: column, C18 silica gel; mobile phase, MeCN in Water (0.1% TFA), 10% to 50% gradient in 10 min; detector, UV 254 nm. This resulted in 3-{4-[(2,5-dioxopyrrol-1-yl)methyl]piperidin-1-yl}benzenesulfonyl fluoride (60 mg, 11.59% yield, 97.9% purity) as a light yellow oil. LCMS:(ES, m/z): [M+H]⁺=353.1.

¹H NMR— (300 MHz, DMSO-d₆) δ 7.56 (t, J=8.2 Hz, 1H), 7.47 (d, J=9.2 Hz, 1H), 7.42-7.35 (m, 2H), 7.05 (s, 2H), 3.85 (d, J=12.9 Hz, 2H), 3.32 (s, 1H), 2.84-2.69 (m, 2H), 1.81 (ddd, J=11.4, 7.6, 3.9 Hz, 1H), 1.66 (d, J=13.1 Hz, 2H), 1.21 (qd, J=12.0, 4.0 Hz, 2H), −0.05 (s, 1H).

### Example 82. Synthesis of 3-((4-(2,5-dioxo-2,5-dihydro-1H-pyrrol-1-yl)piperidin-1-yl)methyl)phenyl sulfurofluoridate formate salt (MFS-8-7)

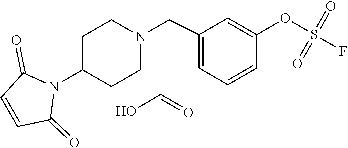

Step 1: To a stirred solution of 3-hydroxybenzaldehyde (1 g, 8.188 mmol, 1 equiv), tert-butyl N-(piperidin-4-yl)carbamate (1.64 g, 8.188 mmol, 1 equiv) and AcOH (0.49 g, 8.188 mmol, 1 equiv) in DCM (15 mL) at room temperature under nitrogen atmosphere. The mixture was stirred for 15 min. sodium bis(acetyloxy)boranuidyl acetate (3.47 g, 16.376 mmol, 2 equiv) was added and stirred for 2 h. The resulting mixture was diluted with 20 mL H₂O and extracted with DCM (15 mL×2). The aqueous phase was basified to pH 7 with saturated NaHCO₃(aq.). The aqueous phase was extracted with EtOAc (15 mL×2). The combined organic layers were washed with brine (15 mL), dried over anhydrous Na2SO4. After filtration, the filtrate was concentrated under reduced pressure. The residue was purified by silica gel column chromatography, eluted with PE/THF (3:1) to afford tert-butyl N-{1-[(3-hydroxyphenyl) methyl]piperidin-4-yl}carbamate (1 g, 39.86% yield) as a light yellow solid.

Step 2: To a stirred solution of tert-butyl N-{1-[(3-hydroxyphenyl) methyl]piperidin-4-yl}carbamate (1 g, 3.264 mmol, 1 equiv) in DCM (5 mL) and HCl in dioxane (5 mL) w at room temperature under nitrogen atmosphere. The resulting mixture was stirred at room temperature for additional 1 h. The resulting mixture was filtered, the filter cake was washed with DCM (3 mL×3). The filtrate was concentrated under reduced pressure. This resulted in 3-[(4-aminopiperidin-1-yl)methyl]phenol hydrochloride (600 mg, 75.73% yield) as a light yellow solid. The crude product was used in the next step directly without further purification.

Step 2: To a stirred solution of 3-[(4-aminopiperidin-1-yl) methyl]phenol hydrochloride (600 mg, 2.472 mmol, 1 equiv) in NaHCO₃(aq.) (2 mL) and 1,4-dioxane (6 mL) was added methyl 2,5-dioxopyrrole-1-carboxylate (575.07 mg, 3.708 mmol, 1.5 equiv) in portions at 0° C. under nitrogen atmosphere. The resulting mixture was stirred at room temperature for additional overnight. The resulting mixture was diluted with 20 mL H2O and extracted with EtoAC (10 mL×3). The combined organic layers were washed with brine (10 mL), dried over anhydrous Na2SO4. After filtration, the filtrate was concentrated under reduced pressure. The residue was purified by reversed-phase flash chromatography with the following conditions: column, C18 silica gel; mobile phase, MeCN in Water (0.1% FA), 10% to 50% gradient in 10 min; detector, UV 254 nm. This resulted in 1-{1-[(3-hydroxyphenyl)methyl]piperidin-4-yl}pyrrole-2,5-dione (300 mg, 42.39% yield) as a light yellow oil.

Step 4: To a stirred solution of 1-{1-[(3-hydroxyphenyl)methyl]piperidin-4-yl}pyrrole-2,5-dione (300 mg, 1.048 mmol, 1 equiv) and 3-(fluorosulfonyl)-1,2-dimethylimidazol-1-ium triflate (515.88 mg, 1.572 mmol, 1.5 equiv) in MeCN (5 mL) were added TEA (212.05 mg, 2.096 mmol, 2 equiv) dropwise at 0° C. under nitrogen atmosphere. The resulting mixture was stirred at room temperature for additional 30 min. The resulting mixture was concentrated under reduced pressure. The residue was purified by reversed-phase flash chromatography with the following conditions: column, C18 silica gel; mobile phase, MeCN in Water (0.1% FA), 10% to 50% gradient in 10 min; detector, UV 254 nm. This resulted in 3-{[4-(2,5-dioxopyrrol-1-yl)piperidin-1-yl]methyl}phenyl sulfurofluoridate (44.6 mg, 11.56% yield, 98.0% purity) as a white solid. LCMS:(ES, m/z): [M+H]⁺=369.1.

¹H NMR (300 MHz, DMSO-d₆) δ 7.79-7.45 (m, 4H), 6.99 (s, 2H), 3.93 (s, 3H), 3.33 (s, 2H), 3.11 (s, 2H), 2.31-2.27 (m, 2H), 1.76-1.67 (m, 2H).

### Example 83. Synthesis of 3-(2-(2,5-dioxo-2,5-dihydro-1H-pyrrol-1-yl)-7-azaspiro[3.5]nonan-7-yl)phenyl sulfurofluoridate (MFS-9-3)

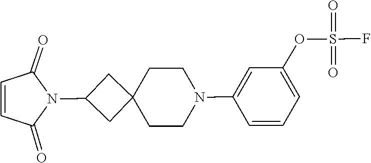 [SMILES: O=C1C=CC(=O)N1C1CC2(CCN(c3cccc(OS(=O)(=O)F)c3)CC2)C1]

Step 1: Into a 40 mL sealed tube were added tert-butyl N-{7-azaspiro[3.5]nonan-2-yl}carbamate (2 g, 8.32 mmol, 1.00 equiv), 1-(benzyloxy)-3-bromobenzene (2.41 g, 9.15 mmol, 1.10 equiv), Pd₂(dba)₃ (0.76 g, 0.83 mmol, 0.10 equiv), XPhos (0.79 g, 1.66 mmol, 0.20 equiv), toluene (20 mL) and Cs₂CO₃ (5.42 g, 16.642 mmol, 2.00 equiv) at room temperature. The resulting mixture was stirred at 100° C. for 2 h under nitrogen atmosphere. The mixture was allowed to cool down to room temperature. The reaction was quenched with water at room temperature. The resulting mixture was extracted with EtOAc (3×100 mL). The combined organic layers were washed with brine (1×50 mL), dried over anhydrous Na₂SO₄. After filtration, the filtrate was concentrated under reduced pressure. The residue was purified by silica gel column chromatography, eluted with PE/EA (1:1) to afford tert-butyl N-{7-[3-(benzyloxy)phenyl]-7-azaspiro[3.5]nonan-2-yl}carbamate (2.5 g, 71.10% yield) as a yellow solid.

Step 2: Into a 40 mL sealed tube were added tert-butyl N-{7-[3-(benzyloxy)phenyl]-7-azaspiro[3.5]nonan-2-yl}carbamate (2.5 g, 5.92 mmol, 1.00 equiv), DCM (5 mL) and HCl in 1,4-dioxane (4.0 M) (5 mL) at room temperature. The resulting mixture was stirred at room temperature for 2 h under nitrogen atmosphere. The resulting mixture was concentrated under vacuum. This resulted in 7-[3-(benzyloxy)phenyl]-7-azaspiro[3.5]nonan-2-amine (1.9 g, 99.60% yield) as a yellow oil.

Step 3: A mixture of 7-[3-(benzyloxy)phenyl]-7-azaspiro[3.5]nonan-2-amine (1.9 g, 5.892 mmol, 1 equiv) and Pd/C (190 mg, 1.785 mmol, 0.30 equiv) in MeOH was stirred at room temperature for 1 h under hydrogen atmosphere. The resulting mixture was filtered, the filter cake was washed with MeOH (2×10 mL). The filtrate was concentrated under reduced pressure. This resulted in 3-{2-amino-7-azaspiro[3.5]nonan-7-yl}phenol (1.3 g, 94.96% yield) as a yellow oil.

Step 4: To a stirred solution of 3-{2-amino-7-azaspiro[3.5]nonan-7-yl}phenol (1.3 g, 5.59 mmol, 1.00 equiv) and maleic anhydride (0.60 g, 6.16 mmol, 1.10 equiv) in ACN (10 mL) was added TEA (1.70 g, 16.79 mmol, 3.00 equiv) dropwise at room temperature under nitrogen atmosphere. The resulting mixture was stirred at room temperature for 1 h under nitrogen atmosphere. The resulting mixture was concentrated under vacuum. The residue was purified by reversed-phase flash chromatography with the following conditions: column, C18 silica gel; mobile phase, MeCN in Water (0.1% FA), 5% to 20% gradient in 10 min; detector, UV 254 nm. This resulted in (2Z)-3-{[7-(3-hydroxyphenyl)-7-azaspiro[3.5]nonan-2-yl]carbamoyl}prop-2-enoic acid (1 g, 54.09% yield) as a yellow solid.

Step 5: Into a 40 mL sealed tube were added (2Z)-3-{[7-(3-hydroxyphenyl)-7-azaspiro[3.5]nonan-2-yl]carbamoyl}prop-2-enoic acid (1 g, 3.03 mmol, 1.00 equiv), AcOH (10 mL) and (2Z)-3-{[7-(3-hydroxyphenyl)-7-azaspiro[3.5]nonan-2-yl]carbamoyl}prop-2-enoic acid (1 g, 3.03 mmol, 1.00 equiv) at room temperature. The resulting mixture was stirred at 100° C. for 16 h under nitrogen atmosphere. The resulting mixture was concentrated under vacuum. The residue was purified by reversed-phase flash chromatography with the following conditions: column, C18 silica gel; mobile phase, MeCN in Water (0.1% FA), 5% to 60% gradient in 15 min; detector, UV 254 nm. This resulted in 1-[7-(3-hydroxyphenyl)-7-azaspiro[3.5]nonan-2-yl]pyrrole-2,5-dione (300 mg, 31.73% yield) as a yellow solid.

Step 6: To a stirred solution of 3-(fluorosulfonyl)-1,2-dimethylimidazol-1-ium triflate (472.88 mg, 1.44 mmol, 1.50 equiv) and 1-[7-(3-hydroxyphenyl)-7-azaspiro[3.5]nonan-2-yl]pyrrole-2,5-dione (300 mg, 0.96 mmol, 1.00 equiv) in ACN (10 mL) was added TEA (291.56 mg, 2.88 mmol, 3.00 equiv) dropwise at 0° C. under nitrogen atmosphere. The resulting mixture was stirred at room temperature for 30 min under nitrogen atmosphere. The residue was purified by silica gel column chromatography, eluted with THF/EA (1:3) to afford 3-[2-(2,5-dioxopyrrol-1-yl)-7-azaspiro[3.5]nonan-7-yl]phenyl sulfurofluoridate (65 mg, 17.16% yield) as a yellow solid. LCMS:(ES, m/z): [M+H]⁺=395.1.

¹H NMR (300 MHz, DMSO-d₆, ppm): δ 7.35 (t, J=8.2 Hz, 1H), 7.10-7.02 (m, 2H), 6.98 (s, 2H), 6.83 (d, J=8.1 Hz, 1H), 4.52 (p, J=8.9 Hz, 1H), 3.21 (dt, J=20.0, 5.6 Hz, 4H), 2.49-2.37 (m, 2H), 2.23-2.08 (m, 2H), 1.71 (t, J=5.5 Hz, 4H).

### Example 84. Synthesis of 5-(2-(2,5-dioxo-2,5-dihydro-1H-pyrrol-1-yl)-7-azaspiro[3.5]nonane-7-carbonyl)-2-fluorophenyl sulfurofluoridate (MFS-10-9)

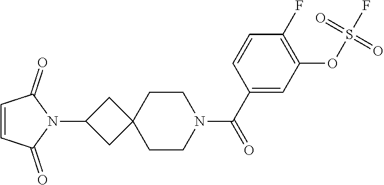 [SMILES: O=C(c1ccc(F)c(OS(=O)(=O)F)c1)N1CCC2(CC1)CC(N1C(=O)C=CC1=O)C2]

Step 1: To a stirred mixture of 4-fluoro-3-hydroxybenzoic acid (2 g, 12.811 mmol, 1 equiv) and [(4-acetamidophenyl) (fluorosulfonyl) amino]sulfonyl fluoride (6.04 g, 19.216 mmol, 1.5 equiv) in THF (20 mL) were added DBU (3.90 g, 25.622 mmol, 2 equiv) in portions at 0° C. The resulting mixture was stirred at room temperature for 1 h. The mixture was acidified neutralized to pH 3 with HCl (3M). The resulting mixture was extracted with CH₂Cl₂ (3×20 mL). The combined organic layers were washed with water (3×20 mL), dried over anhydrous Na₂SO₄. After filtration, the filtrate was concentrated under reduced pressure. The residue was purified by reversed-phase flash chromatography with the following conditions: column, C18 silica gel; mobile phase, MeCN in Water (0.1% FA), 10% to 90% gradient in 10 min; detector, UV 254 nm. This resulted in 4-fluoro-3-[(fluorosulfonyl) oxy]benzoic acid (1.8 g, 58.99% yield) as a white solid.

Step 2: To a stirred mixture of 4-fluoro-3-[(fluorosulfonyl) oxy]benzoic acid (389.24 mg, 1.634 mmol, 1.2 equiv), HATU (776.79 mg, 2.043 mmol, 1.5 equiv) and DIEA (528.08 mg, 4.086 mmol, 3 equiv) in DCM (5 mL) at room temperature under nitrogen atmosphere. The mixture was stirred for 15 min. 1-{7-azaspiro [3.5]nonan-2-yl}pyrrole-2,5-dione (300 mg, 1.362 mmol, 1 equiv) was added and stirred for 30 min. The resulting mixture was extracted with CH₂Cl₂ (3×5 mL). The combined organic layers were washed with water (3×5 mL), dried over anhydrous Na₂SO₄. After filtration, the filtrate was concentrated under reduced pressure. The residue was purified by reversed-phase flash chromatography with the following conditions: column, C18 silica gel; mobile phase, MeCN in Water (0.1% FA), 10% to 95% gradient in 10 min; detector, UV 254 nm. This resulted in 5-[2-(2,5-dioxopyrrol-1-yl)-7-azaspiro [3.5]nonane-7-carbonyl]-2-fluorophenyl sulfurofluoridate (100 mg, 16.67% yield, 100.0% purity) as a white solid. LCMS:(ES, m/z): [M+H]⁺=441.0

¹H NMR (300 MHz, DMSO-d₆, ppm) δ 7.98-7.90 (m, 1H), 7.70 (dd, J=10.2, 8.6 Hz, 1H), 7.60 (ddd, J=8.6, 4.8, 2.0 Hz, 1H), 6.98 (s, 2H), 4.59-4.41 (m, 1H), 3.53 (s, 2H), 3.24 (s, 2H), 2.42 (d, J=10.0 Hz, 2H), 2.17 (d, J=10.1 Hz, 2H), 1.63 (s, 4H).

### Example 85. Synthesis of 2-((2,5-dioxo-2,5-dihydro-1H-pyrrol-1-yl)methyl)phenyl sulfurofluoridate (MFS-3-1)

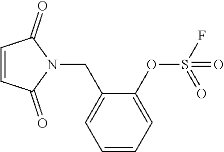 [SMILES: O=C1C=CC(=O)N1Cc1ccccc1OS(=O)(=O)F]

Step 1: To a stirred solution of 2-(aminomethyl) phenol (2 g, 16.24 mmol, 1.00 equiv) and maleic anhydride (4.78 g, 48.72 mmol, 3.00 equiv) in HOAc (20 mL) at room temperature under nitrogen atmosphere. The resulting mixture was stirred at 90° C. for additional overnight. The mixture was allowed to cool down to room temperature. The resulting mixture was concentrated under reduced pressure. The residue was purified by reversed-phase flash chromatography with the following conditions: column, C18 silica gel; mobile phase, MeCN in Water (0.1% FA), 10% to 50% gradient in 10 min; detector, UV 254 nm. This resulted in 1-[(2-hydroxyphenyl) methyl]pyrrole-2,5-dione (1.1 g, 33.33% yield) as a light yellow oil.

Step 2: To a stirred solution of 1-[(2-hydroxyphenyl) methyl]pyrrole-2,5-dione (1.1 g, 5.41 mmol, 1.00 equiv) and 3-(fluorosulfonyl)-1,2-dimethylimidazol-1-ium triflate (2.13 g, 6.49 mmol, 1.20 equiv) in MeCN (20 mL) were added TEA (1.10 g, 10.83 mmol, 2.00 equiv) dropwise at 0° C. The resulting mixture was stirred at room temperature for additional 1 h. The resulting mixture was concentrated under vacuum. The residue was purified by reversed-phase flash chromatography with the following conditions: column, C18 silica gel; mobile phase, MeCN in Water (0.1% FA), 10% to 50% gradient in 10 min; detector, UV 254 nm. This resulted in 2-[(2,5-dioxopyrrol-1-yl) methyl]phenyl sulfurofluoridate (400 mg, 25.90% yield) as a light yellow solid. GCMS: GC-MS: (EI+, m/z, M⁺)=285.0.

¹H NMR (400 MHz, DMSO-d₆, ppm): δ 7.61 (dt, J=8.0, 1.8 Hz, 1H), 7.57-7.46 (m, 3H), 7.13-7.10 (m, 2H), 4.71 (s, 2H).

### Example 86. Synthesis of 3-(2,5-dioxo-2,5-dihydro-1H-pyrrol-1-yl)phenyl sulfurofluoridate (MFS-3-2)

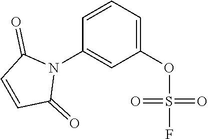 [SMILES: O=C1C=CC(=O)N1c1cccc(OS(=O)(=O)F)c1]

Step 1: Into a 40 mL sealed tube were added m-aminophenol (1 g, 9.16 mmol, 1.00 equiv), THE (10 mL) and 1-(fluorosulfonyl)-2,3-dimethyl-1H-imidazol-3-ium trifluoromethanesulfonate (3.91 g, 11.913 mmol, 1.30 equiv) at room temperature. To the above mixture was added DBU (1.40 g, 9.16 mmol, 1.00 equiv) dropwises over 2 min at 0° C. The resulting mixture was stirred at room temperature for additional 2 h. The residue was purified by silica gel column chromatography, eluted with PE/THF (10:1) to afford 3-aminophenyl sulfurofluoridate (600 mg, 34.25% yield, 95% purity) as a yellow oil.

Step 2: Into a 40 mL sealed tube were added 3-aminophenyl sulfurofluoridate (600 mg, 3.138 mmol, 1 equiv), acetic acid (20 mL) and maleic anhydride (0.37 g, 3.766 mmol, 1.2 equiv) at room temperature. The resulting mixture was stirred at 110° C. for overnight under nitrogen atmosphere. The mixture was allowed to cool down to room temperature. The residue was purified by silica gel column chromatography, eluted with PE/EA (6:1) to afford 3-(2,5-dioxopyrrol-1-yl)phenyl sulfurofluoridate (280 mg, 32.89% yield, 99.96% purity) as a yellow solid. GCMS: GC-MS: (EI+, m/z, M⁺)=271.0.

¹H NMR (300 MHz, DMSO-d₆, ppm): δ 7.81-7.63 (m, 3H), 7.56 (dt, J=7.8, 1.4 Hz, 1H), 7.25 (s, 2H).

### Example 87. Synthesis of 5-(dimethylcarbamoyl)-2-((2,5-dioxo-2,5-dihydro-1H-pyrrol-1-yl)methyl)phenyl sulfurofluoridate (MFS-3-8)

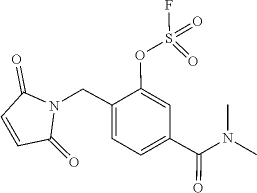 [SMILES: CN(C)C(=O)c1ccc(CN2C(=O)C=CC2=O)c(OS(=O)(=O)F)c1]

Step 1: A solution of methyl 4-cyano-3-hydroxybenzoate (3 g, 16.93 mmol, 1 equiv) and KOH (2.85 g, 50.80 mmol, 3 equiv) in EtOH (50 mL) was stirred at 90° C. for overnight under nitrogen atmosphere. The mixture was allowed to cool down to room temperature. The resulting mixture was concentrated under reduced pressure. The residue was purified by reversed-phase flash chromatography with the following conditions: column, C18 silica gel; mobile phase, MeCN in Water (0.1% FA), 10% to 50% gradient in 10 min; detector, UV 254 nm. This resulted in 4-cyano-3-hydroxybenzoic acid (1.6 g, 57.92% yield, 95% purity) as a yellow solid.

Step 2: A solution of 4-cyano-3-hydroxybenzoic acid (1.6 g, 9.80 mmol, 1.00 equiv), HATU (7.46 g, 19.61 mmol, 2 equiv), DIEA (5.13 mL, 29.42 mmol, 3 equiv) and 4-cyano-3-hydroxybenzoic acid (1.6 g, 9.80 mmol, 1.00 equiv) in N,N-dimethylacetamide (20 mL) was stirred at room temperature for 3 h under nitrogen atmosphere. The resulting mixture was concentrated under reduced pressure. The residue was purified by reversed-phase flash chromatography with the following conditions: column, C18 silica gel; mobile phase, MeCN in Water (0.1% FA), 10% to 50% gradient in 10 min; detector, UV 254 nm. This resulted in 4-cyano-3-hydroxy-N,N-dimethylbenzamide (890 mg, 47.71% yield, 95% purity) as a yellow oil.

Step 3: A solution of 4-cyano-3-hydroxy-N,N-dimethylbenzamide (890 mg, 4.67 mmol, 1 equiv) and NiCl₂·6H₂O (111.22 mg, 0.46 mmol, 0.1 equiv) in MeOH (20 mL) was stirred at 0° C. for 3 min under nitrogen atmosphere. To the above mixture was added NaBH₄ (1062.09 mg, 28.07 mmol, 6 equiv) in portions over 3 min at 0° C. The resulting mixture was stirred at 0° C. for additional 30 min. The resulting mixture was concentrated under reduced pressure. The residue was purified by reversed-phase flash chromatography with the following conditions: column, C18 silica gel; mobile phase, MeCN in Water (0.1% FA), 10% to 50% gradient in 10 min; detector, UV 254 nm. This resulted in 4-(aminomethyl)-3-hydroxy-N,N-dimethylbenzamide (600 mg, 66.02% yield, 95% purity) as a yellow oil.

Step 4: A solution of maleic anhydride (908.71 mg, 9.26 mmol, 3 equiv) and 4-(aminomethyl)-3-hydroxy-N,N-dimethylbenzamide (600 mg, 3.08 mmol, 1.00 equiv) in HOAc (10 mL) was stirred at 90° C. for overnight under nitrogen atmosphere. The resulting mixture was concentrated under reduced pressure. The residue was purified by reversed-phase flash chromatography with the following conditions: column, C18 silica gel; mobile phase, MeCN in Water (0.1% FA), 10% to 50% gradient in 10 min; detector, UV 254 nm. This resulted in 4-(aminomethyl)-3-hydroxy-N,N-dimethylbenzamide (600 mg, 66.02% yield, 95% purity) as a yellow oil.

Step 5: A solution of 4-[(2,5-dioxopyrrol-1-yl) methyl]-3-hydroxy-N,N-dimethylbenzamide (220 mg, 0.80 mmol, 1 equiv), 1-(fluorosulfonyl)-2,3-dimethyl-1H-imidazol-3-ium trifluoromethanesulfonate (342.28 mg, 1.04 mmol, 1.3 equiv) and Et₃N (243.50 mg, 2.40 mmol, 3 equiv) in ACN (10 mL) was stirred at 0° C. for 30 min under nitrogen atmosphere. The residue was purified by reversed-phase flash chromatography with the following conditions: column, C18 silica gel; mobile phase, MeCN in Water (0.1% FA), 10% to 50% gradient in 10 min; detector, UV 254 nm. This resulted in 5-(dimethylcarbamoyl)-2-[(2,5-dioxopyrrol-1-yl) methyl]phenyl sulfurofluoridate (84.4 mg, 29.53% yield, 99.1% purity) as a white solid. LCMS:(ES, m/z): [M+H]⁺=357.1.

¹H NMR (300 MHz, DMSO-d₆, ppm) δ 7.67 (dt, J=1.9, 0.9 Hz, 1H), 7.53 (d, J=1.0 Hz, 2H), 7.12 (s, 2H), 4.73 (s, 2H), 2.93 (d, J=30.3 Hz, 6H).

### Example 88. Synthesis of 2-(1-(2,5-dioxo-2,5-dihydro-1H-pyrrol-1-yl)-2-(4-methylpiperazin-1-yl)ethyl) phenyl sulfurofluoridate (MFS-3-16)

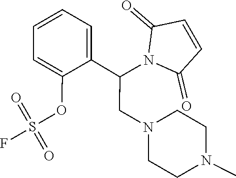 [SMILES: CN1CCN(CC(c2ccccc2OS(=O)(=O)F)N2C(=O)C=CC2=O)CC1]

Step 1: To a stirred solution of 2-bromo-1-(2-hydroxyphenyl)ethanone (2 g, 9.300 mmol, 1 equiv), 1-methyl-piperazine (4.66 g, 46.500 mmol, 5 equiv), K₂CO₃ (6426.76 mg, 46.500 mmol, equiv) and KI (0.15 g, 0.930 mmol, 0.1 equiv) in DCM (30 mL) at room temperature under nitrogen atmosphere. The resulting mixture was stirred at room temperature for additional 2 h. The resulting mixture was diluted with 30 mL H₂O and extracted with DCM (30 mL×3). The combined organic layers were washed with brine (30 mL) dried over anhydrous Na₂SO₄. After filtration, the filtrate was concentrated under reduced pressure. The residue was purified by silica gel column chromatography, eluted with PE/THF (1:1) to afford 1-(2-hydroxyphenyl)-2-(4-methylpiperazin-1-yl)ethanone (2 g, 91.78% yield) as a light yellow oil.

Step 2: To a stirred solution of 1-(2-hydroxyphenyl)-2-(4-methylpiperazin-1-yl) ethanone (2 g, 8.536 mmol, 1 equiv) and hydroxylamine hydrochloride (1.48 g, 21.340 mmol, 2.5 equiv) in pyridine (20 mL) at room temperature under nitrogen atmosphere. The resulting mixture was stirred at 50° C. for additional 2 h. The mixture was allowed to cool down to room temperature. The resulting mixture was diluted with 30 mL H₂O and extracted with EtOAc (30 mL×3). The combined organic layers were washed with brine (30 mL) dried over anhydrous Na₂SO₄. After filtration, the filtrate was concentrated under reduced pressure. The residue was purified by silica gel column chromatography, eluted with PE/THF (3:1) to afford 2-[(1E)-1-(hydroxyimino)-2-(4-methylpiperazin-1-yl)ethyl]phenol (920 mg, 43.23% yield) as a light

Step 3: To a stirred solution of 2-[(1E)-1-(hydroxyimino)-2-(4-methylpiperazin-1-yl)ethyl]phenol (920 mg, 3.690 mmol, 1 equiv) in THF (10 mL) were added Lithium aluminum hydride (2.4 M in THF) (3.08 mL, 7.380 mmol, 2 equiv) dropwise at 0° C. under nitrogen atmosphere. The resulting mixture was stirred at 50° C. for additional 2 h. The mixture was allowed to cool down to room temperature. The reaction was quenched with disodium decahydrate sulfate at 0° C. The resulting mixture was filtered, the filter cake was washed with EtOAc (3 mL×3). The residue was diluted with 10 mL H₂O and extracted with EtOAc (10 mL×3). The combined organic layers were washed with brine (10 mL) dried over anhydrous Na₂SO₄. After filtration, the filtrate was concentrated under reduced pressure The residue was purified by reversed-phase flash chromatography with the following conditions: column, C18 silica gel; mobile phase, MeCN in Water (0.1% FA), 10% to 50% gradient in 10 min; detector, UV 254 nm. This resulted in 2-[1-amino-2-(4-methylpiperazin-1-yl)ethyl]phenol (200 mg, 23.03% yield) as a light brown solid.

Step 4: To a stirred solution of 2-[1-amino-2-(4-methylpiperazin-1-yl)ethyl]phenol (200 mg, 0.850 mmol, 1 equiv) and maleic anhydride (100.00 mg, 1.020 mmol, 1.2 equiv) in AcOH (3 mL) at room temperature under nitrogen atmosphere. The resulting mixture was stirred at 120° C. for additional overnight. The mixture was allowed to cool down to room temperature. The resulting mixture was diluted with 3 mL H₂O and extracted with EtOAc (3 mL×3). The combined organic layers were washed with brine (3 mL) dried over anhydrous Na₂SO₄. After filtration, the filtrate was concentrated under reduced pressure. The residue was purified by reversed-phase flash chromatography with the following conditions: column, C18 silica gel; mobile phase, MeCN in Water (0.1% FA), 10% to 80% gradient in 10 min; detector, UV 254 nm. This resulted in 1-[1-(2-hydroxyphenyl)-2-(4-methylpiperazin-1-yl)ethyl]pyrrole-2,5-dione (90 mg, 33.58% yield) as a light yellow oil.

Step 5: To a stirred solution of 1-[1-(2-hydroxyphenyl)-2-(4-methylpiperazin-1-yl)ethyl]pyrrole-2,5-dione (90 mg, 0.285 mmol, 1 equiv) and 3-(fluorosulfonyl)-1,2-dimethylimidazol-1-ium triflate (140.51 mg, 0.427 mmol, 1.5 equiv) in MeCN (2 mL) were added TEA (57.76 mg, 0.570 mmol, 2 equiv) dropwise at 0° C. under nitrogen atmosphere. The resulting mixture was stirred at room temperature for additional 30 min. The resulting mixture was concentrated under reduced pressure. The residue was purified by reversed-phase flash chromatography with the following conditions: column, C18 silica gel; mobile phase, MeCN in Water (0.1% FA), 10% to 80% gradient in 10 min; detector, UV 254 nm. This resulted in 2-[1-(2,5-dioxopyrrol-1-yl)-2-(4-methylpiperazin-1-yl)ethyl]phenyl sulfurofluoridate (15 mg, 13.23% yield, 95.1% purity) as a light yellow oil. LCMS:(ES, m/z): [M+H]⁺=398.1.

¹H NMR (300 MHz, DMSO-d₆, ppm) δ 7.86-7.77 (m, 1H), 7.59-7.52 (m, 3H), 7.05 (s, 2H), 5.52 (dd, J=10.9, 5.2 Hz, 1H), 3.43-3.31 (m, 2H), 2.69 (dd, J=12.8, 5.2 Hz, 1H), 2.58-2.51 (m, 6H).2.37-2.11 (m, 6H).

### Example 89. Synthesis of 3-((2,5-dioxo-2,5-dihydro-1H-pyrrol-1-yl)methyl)phenyl sulfurofluoridate (MFS-4-1)

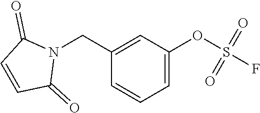 [SMILES: O=C1C=CC(=O)N1Cc1cccc(OS(=O)(=O)F)c1]

Step 1: To a stirred solution of 3-(aminomethyl) phenol (500 mg, 4.060 mmol, 1 equiv) and maleic anhydride (1194.31 mg, 12.180 mmol, 3 equiv) in AcOH (5 mL) at room temperature under nitrogen atmosphere. The resulting mixture was stirred at 90° C. for overnight. The mixture was allowed to cool down to room temperature. The resulting mixture was concentrated under reduced pressure. The residue was purified by reversed-phase flash chromatography with the following conditions: column, C18 silica gel; mobile phase, MeCN in Water (0.1% FA), 10% to 50% gradient in 10 min; detector, UV 254 nm. This resulted in 2-[(3-hydroxyphenyl) methyl]cyclopent-4-ene-1,3-dione (300 mg, 36.54% yield) as a light yellow oil.

Step 2: To a stirred solution of 2-[(3-hydroxyphenyl) methyl]cyclopent-4-ene-1,3-dione (300 mg, 1.484 mmol, 1 equiv) and 3-(fluorosulfonyl)-1,2-dimethylimidazol-1-ium triflate (584.4 mg, 1.781 mmol, 1.2 equiv) in MeCN (10 mL) were added TEA (300.26 mg, 2.968 mmol, 2 equiv) dropwise at 0° C. The resulting mixture was stirred at room temperature for 1 h. The resulting mixture was concentrated under vacuum. The residue was purified by reversed-phase flash chromatography with the following conditions: column, C18 silica gel; mobile phase, MeCN in Water (0.1% FA), 10% to 50% gradient in 10 min; detector, UV 254 nm. This resulted in 3-[(2,5-dioxocyclopent-3-en-1-yl) methyl]phenyl sulfurofluoridate (73 mg, 17.31% yield, 96.8% purity) as a light yellow solid. GCMS: GC-MS: (EI+, m/z, M⁺)=285.0.

¹H NMR (400 MHz, DMSO-d₆, ppm) δ 7.61-7.47 (m, 3H), 7.39 (d, J=7.5 Hz, 1H), 7.11 (d, J=1.1 Hz, 2H), 4.69 (s, 2H).

### Example 90. Synthesis of 3-(dimethylcarbamoyl)-5-((2,5-dioxo-2,5-dihydro-1H-pyrrol-1-yl)methyl)phenyl sulfurofluoridate (MFS-4-12)

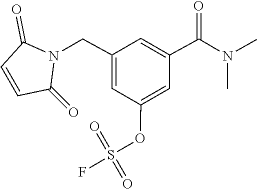 [SMILES: CN(C)C(=O)c1cc(CN2C(=O)C=CC2=O)cc(OS(=O)(=O)F)c1]

Step 1: To a stirred solution of 3-cyano-5-hydroxybenzoic acid (1.5 g, 9.195 mmol, 1 equiv), dimethylamine (0.62 g, 13.793 mmol, 1.5 equiv) and DIEA (2.38 g, 18.390 mmol, 2 equiv) in DMAc (20 mL) was added Propanephosphonic acid cyclic anhydride (50% in ethyl acetate) (8.78 g, 13.793 mmol, 1.5 equiv, 50%) dropwise at 0° C. under nitrogen atmosphere. The resulting mixture was diluted with 30 mL H₂O and extracted with EtOAc (30 mL×3). The combined organic layers were washed with brine (30 mL) dried over anhydrous Na₂SO₄. After filtration, the filtrate was concentrated under reduced pressure. The residue was purified by silica gel column chromatography, eluted with PE/THF (3:1) to afford 3-cyano-5-hydroxy-N,N-dimethylbenzamide (1 g, 57.18% yield) as a light yellow oil.

Step 2: To a stirred solution of 3-cyano-5-hydroxy-N,N-dimethylbenzamide (1 g, 5.258 mmol, 1 equiv) in THF (10 mL) was added Lithium aluminum hydriden (2.4 M in THF) (4.38 mL, 10.516 mmol, 2 equiv) dropwise at 0° C. under nitrogen atmosphere. The resulting mixture was stirred at 50° C. for additional 2 h. The mixture was allowed to cool down to room temperature. The reaction was quenched with disodium decahydrate sulfate at 0° C. The resulting mixture was filtered, the filter cake was washed with EtOAc (10 mL×3). The residue was diluted with 20 mL H2O and extracted with EtOAc (20 mL×3). The combined organic layers were washed with brine (20 mL) dried over anhydrous Na₂SO₄. After filtration, the filtrate was concentrated under reduced pressure. The residue was purified by reversed-phase flash chromatography with the following conditions: column, C18 silica gel; mobile phase, MeCN in Water (0.1% FA), 10% to 45% gradient in 10 min; detector, UV 254 nm. This resulted in 3-(aminomethyl)-5-hydroxy-N,N-dimethylbenzamide (500 mg, 48.96% yield) as a yellow oil.

Step 3: To a stirred solution of 3-(aminomethyl)-5-hydroxy-N,N-dimethylbenzamide (500 mg, 2.574 mmol, 1 equiv) and maleic anhydride (504.84 mg, 5.148 mmol, 2 equiv) in AcOH (5 mL) at room temperature under nitrogen atmosphere. The resulting mixture was stirred at 90° C. for additional 5 h. The mixture was allowed to cool down to room temperature. The resulting mixture was concentrated under reduced pressure. The residue was purified by reversed-phase flash chromatography with the following conditions: column, C18 silica gel; mobile phase, MeCN in Water (0.1% FA), 10% to 70% gradient in 10 min; detector, UV 254 nm. This resulted in 3-[(2,5-dioxopyrrol-1-yl)methyl]-5-hydroxy-N,N-dimethylbenzamide (300 mg, 42.49% yield) as a light yellow oil.

Step 4: To a stirred solution of 3-[(2,5-dioxopyrrol-1-yl)methyl]-5-hydroxy-N,N-dimethylbenzamide (300 mg, 1.094 mmol, 1 equiv) and 3-(fluorosulfonyl)-1,2-dimethylimidazol-1-ium triflate (538.55 mg, 1.641 mmol, 1.5 equiv) in MeCN (5 mL) were added TEA (221.37 mg, 2.188 mmol, 2 equiv) dropwise at 0° C. under nitrogen atmosphere. The resulting mixture was stirred at room temperature for additional 30 min. The resulting mixture was concentrated under reduced pressure. The residue was purified by reversed-phase flash chromatography with the following conditions: column, C18 silica gel; mobile phase, MeCN in Water (0.1% FA), 10% to 80% gradient in 10 min; detector, UV 254 nm. This resulted in 3-(dimethylcarbamoyl)-5-[(2,5-dioxopyrrol-1-yl)methyl]phenyl sulfurofluoridate (51.2 mg, 13.14% yield, 100% purity) as a white oil. LCMS:(ES, m/z): [M+H]⁺=357.0.

¹H NMR (300 MHz, DMSO-d₆, ppm) δ 7.60 (t, J=2.4 Hz, 2H), 7.42 (t, J=1.5 Hz, 1H), 7.11 (s, 2H), 4.72 (s, 2H), 2.99 (s, 3H), 2.86 (s, 3H).

### Example 91. Synthesis of 4-((2,5-dioxo-2,5-dihydro-1H-pyrrol-1-yl)methyl)phenyl sulfurofluoridate (MFS-5-1)

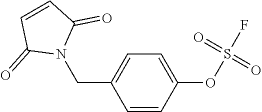 [SMILES: O=C1C=CC(=O)N1Cc1ccc(OS(=O)(=O)F)cc1]

Step 1: To a stirred solution of 4-(aminomethyl)phenol (2 g, 16.24 mmol, 1.00 equiv) and maleic anhydride (4.78 g, 48.72 mmol, 3.00 equiv) in HOAc (20 mL) at room temperature under nitrogen atmosphere. The resulting mixture was stirred at 90° C. for additional overnight. The mixture was allowed to cool down to room temperature. The resulting mixture was concentrated under reduced pressure. The residue was purified by reversed-phase flash chromatography with the following conditions: column, C18 silica gel; mobile phase, MeCN in Water (0.1% FA), 10% to 50% gradient in 10 min; detector, UV 254 nm. This resulted in 1-(4-hydroxybenzyl)-1H-pyrrole-2,5-dione (1.1 g, 33.33% yield) as a light yellow oil.

Step 2: To a stirred solution of 1-(4-hydroxybenzyl)-1H-pyrrole-2,5-dione (1.1 g, 5.41 mmol, 1.00 equiv) and 3-(fluorosulfonyl)-1,2-dimethylimidazol-1-ium triflate (2.13 g, 6.49 mmol, 1.20 equiv) in MeCN (20 mL) were added TEA (1.10 g, 10.83 mmol, 2.00 equiv) dropwise at 0° C. The resulting mixture was stirred at room temperature for additional 1 h. The resulting mixture was concentrated under vacuum. The residue was purified by reversed-phase flash chromatography with the following conditions: column, C18 silica gel; mobile phase, MeCN in Water (0.1% FA), 10% to 50% gradient in 10 min; detector, UV 254 nm. This resulted in 4-((2,5-dioxo-2,5-dihydro-1H-pyrrol-1-yl) methyl)phenyl sulfurofluoridate (400 mg, 25.90% yield) as a light yellow solid. GCMS: GC-MS: (EI+, m/z, M⁺)=285.0.

¹H NMR (400 MHz, DMSO-d₆, ppm) δ 7.62-7.51 (m, 2H), 7.52-7.42 (m, 2H), 7.10 (s, 2H), 4.66 (s, 2H).

### Example 92. Synthesis of 4-((2,5-dioxo-2,5-dihydro-1H-pyrrol-1-yl)methyl)-2-fluorophenyl sulfurofluoridate (MFS-5-3)

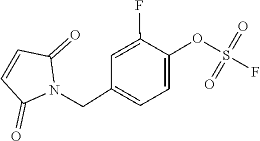 [SMILES: O=C1C=CC(=O)N1Cc1ccc(OS(=O)(=O)F)c(F)c1]

Step 1: To a stirred solution of 2-fluoro-4-methylphenol (1.5 g, 11.892 mmol, 1 equiv) and [(4-acetamidophenyl)(fluorosulfonyl)amino]sulfonyl fluoride (4.49 g, 14.270 mmol, 1.2 equiv) in THF (20 mL) were added DBU (3.98 g, 26.162 mmol, 2.2 equiv) dropwise at 0° C. under nitrogen atmosphere. The resulting mixture was stirred at room temperature for additional 2 h. The mixture was acidified to pH 6 with HCl (1 mol). The resulting mixture was diluted with 30 mL H₂O and extracted with EtOAc (20 mL×3). The combined organic layers were washed with brine (20 mL), dried over anhydrous Na₂SO₄. After filtration, the filtrate was concentrated under reduced pressure. The residue was purified by silica gel column chromatography, eluted with PE/THF (6:1) to afford 2-fluoro-4-methylphenyl sulfurofluoridate (1.3 g, 52.51% yield) as a light yellow oil.

Step 2: To a stirred solution of 2-fluoro-4-methylphenyl sulfurofluoridate (1.3 g, 6.245 mmol, 1 equiv) and benzoyl benzenecarboperoxoate (0.15 g, 0.625 mmol, 0.1 equiv) in CCl₄ (15 mL) were added NBS (1.11 g, 6.245 mmol, 1 equiv) in portions at 0° C. under nitrogen atmosphere. The resulting mixture was stirred at 70° C. for additional 2 h. The mixture was allowed to cool down to room temperature. The resulting mixture was diluted with 15 mL H₂O and extracted with DCM (15 mL×3). The combined organic layers were washed with brine (15 mL), dried over anhydrous Na₂SO₄. After filtration, the filtrate was concentrated under reduced pressure. The residue was purified by reversed-phase flash chromatography with the following conditions: column, C18 silica gel; mobile phase, MeCN in Water (0.1% FA), 10% to 95% gradient in 10 min; detector, UV 254 nm. This resulted in 4-(bromomethyl)-2-fluorophenyl sulfurofluoridate (200 mg, 11.16% yield) as a light yellow oil.

Step 3: To a stirred solution of 4-(bromomethyl)-2-fluorophenyl sulfurofluoridate (200 mg, 0.697 mmol, 1 equiv), maleimide (67.63 mg, 0.697 mmol, 1 equiv) and K₂CO₃ (192.57 mg, 1.394 mmol, 2 equiv) in DMF (3 mL) at room temperature under nitrogen atmosphere. The resulting mixture was stirred at room temperature for additional 3 h. The resulting mixture was diluted with 3 mL H₂O and extracted with EtOAc (2 mL×3). The combined organic layers were washed with brine (3 mL), dried over anhydrous Na₂SO₄. After filtration, the filtrate was concentrated under reduced pressure. The residue was purified by reversed-phase flash chromatography with the following conditions: column, C18 silica gel; mobile phase, MeCN in Water (0.1% FA), 10% to 65% gradient in 10 min; detector, UV 254 nm. This resulted in 4-[(2,5-dioxopyrrol-1-yl)methyl]-2-fluorophenyl sulfurofluoridate (24 mg, 11.36% yield, 100.0% purity) as a light yellow solid. GCMS:(ES, m/z): [M]⁺=302.9.

¹H NMR (300 MHz, DMSO-d₆, ppm) δ 7.84-7.72 (m, 1H), 7.52 (dd, J=11.1, 2.1 Hz, 1H), 7.27 (dd, J=8.5, 2.1 Hz, 1H), 7.10 (s, 2H), 4.67 (s, 2H).

### Example 93. Synthesis of 4-((2,5-dioxo-2,5-dihydro-1H-pyrrol-1-yl)methyl)-2,5-difluorphenyl sulfurofluoridate (MFS-5-4)

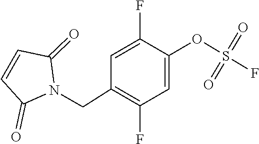 [SMILES: O=C1C=CC(=O)N1Cc1cc(F)c(OS(=O)(=O)F)cc1F]

Step 1: To a stirred mixture of 4-bromo-2,5-difluorophenol (2 g, 9.570 mmol, 1 equiv), potassium tert-butyl N-[(trifluoroboranuidyl)methyl]carbamate (4.54 g, 19.140 mmol, 2 equiv) and Cs₂CO₃ (9.35 g, 28.710 mmol, 3 equiv) in Dioxane (25 mL) and water (5 mL) were added Catacxium A (0.69 g, 1.914 mmol, 0.2 equiv) and Pd(OAc)₂ (0.43 g, 1.914 mmol, 0.2 equiv) in portions at room temperature under nitrogen atmosphere. The resulting mixture was stirred at 100° C. for 1 h under nitrogen atmosphere. The resulting mixture was filtered, the filter cake was washed with MeOH (3×10 mL). The filtrate was concentrated under reduced pressure. The residue was purified by reversed-phase flash chromatography with the following conditions: column, C18 silica gel; mobile phase, MeCN in Water (0.1% FA), 10% to 95% gradient in 15 min; detector, UV 254 nm. This resulted in tert-butyl N-[(2,5-difluoro-4-hydroxyphenyl)methyl]carbamate (1.7 g, 68.52% yield) as a white solid.

Step 2: To a stirred mixture of tert-butyl N-[(2,5-difluoro-4-hydroxyphenyl)methyl]carbamate (1 g, 3.857 mmol, 1 equiv) and 3-(fluorosulfonyl)-1,2-dimethylimidazol-1-ium triflate (1.52 g, 4.628 mmol, 1.2 equiv) in ACN (15 mL) was added TEA (0.59 g, 5.786 mmol, 1.5 equiv) dropwise at 0° C. The resulting mixture was stirred at 0° C. for 30 min. The resulting mixture was extracted with CH₂Cl₂ (3×15 mL). The combined organic layers were washed with water (3×15 mL), dried over anhydrous Na₂SO₄. After filtration, the filtrate was concentrated under reduced pressure. The residue was purified by reversed-phase flash chromatography with the following conditions: column, C18 silica gel; mobile phase, MeCN in Water (0.1% FA), 10% to 95% gradient in 10 min; detector, UV 254 nm. This resulted in tert-butyl N-({2,5-difluoro-4-[(fluorosulfonyl)oxy]phenyl}methyl)carbamate (850 mg, 64.57% yield) as a white solid.

Step 3: A solution of tert-butyl N-({2,5-difluoro-4-[(fluorosulfonyl)oxy]phenyl}methyl)carbamate (500 mg, 1.465 mmol, 1 equiv) in DCM (3 mL) in HCl in 1,4-dioxane (4.0 M) (1 mL) was stirred at room temperature for 1 h under. The resulting mixture was concentrated under reduced pressure to afford 4-(aminomethyl)-2,5-difluorophenyl sulfurofluoridate hydrochloride (380 mg, 93.43% yield) as a white solid. The crude resulting mixture was used in the next step directly without further purification.

Step 4: A solution of 4-(aminomethyl)-2,5-difluorophenyl sulfurofluoridate hydrochloride (300 mg, 1.081 mmol, 1 equiv) in Dioxane (6 mL) and 50% NaHCO₃ aqueous solution (2 mL). To the above mixture was added methyl 2,5-dioxopyrrole-1-carboxylate (201.12 mg, 1.297 mmol, 1.2 equiv) in portions at 0° C. The resulting mixture was stirred at 0° C. for additional 30 min. The resulting mixture was extracted with CH₂Cl₂ (3×10 mL). The combined organic layers were washed with water (3×10 mL), dried over anhydrous Na₂SO₄. After filtration, the filtrate was concentrated under reduced pressure. The residue was purified by reversed-phase flash chromatography with the following conditions: column, C18 silica gel; mobile phase, MeCN in Water (0.1% FA), 10% to 95% gradient in 10 min; detector, UV 254 nm. This resulted in 4-[(2,5-dioxopyrrol-1-yl)methyl]-2,5-difluorophenyl sulfurofluoridate (100 mg, 30.03% yield, 97.6% ¹H NMR (300 MHz, DMSO-d₆, ppm) δ 8.01 (dd, J=9.3, 6.2 Hz, 1H), 7.65 (dd, J=10.5, 6.7 Hz, 1H), 7.10 (s, 2H), 4.67 (s, 2H).

### Example 94. Synthesis of 4-((2,5-dioxo-2,5-dihydro-1H-pyrrol-1-yl)methyl)-2-((4-methylpiperazin-1-yl)methyl)phenyl sulfurofluoridate (MFS-5-7)

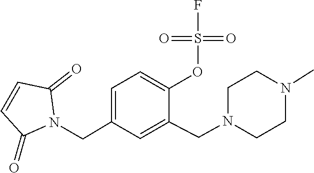 [SMILES: CN1CCN(Cc2cc(CN3C(=O)C=CC3=O)ccc2OS(=O)(=O)F)CC1]

Step 1: To a stirred mixture of 5-bromo-2-hydroxybenzaldehyde (2 g, 9.949 mmol, 1 equiv) in 1,2-DCE (30 mL) was added STAB (4.22 g, 19.898 mmol, 2 equiv) dropwise at 0° C. The resulting mixture was stirred at room temperature for additional 30 min The resulting mixture was extracted with CH₂Cl₂ (3×30 mL). The combined organic layers were washed with water (3×30 mL), dried over anhydrous Na₂SO₄. After filtration, the filtrate was concentrated under reduced pressure. The residue was purified by reversed-phase flash chromatography with the following conditions: column, C18 silica gel; mobile phase, MeCN in Water (0.1% FA), 10% to 80% gradient in 10 min; detector, UV 254 nm. This resulted in 4-bromo-2-[(4-methylpiperazin-1-yl) methyl]phenol (1.5 g, 52.87% yield) as a yellow solid.

Step 2: To a stirred mixture of 4-bromo-2-[(4-methylpiperazin-1-yl) methyl]phenol (1.2 g, 4.208 mmol, 1 equiv) and potassium tert-butyl N-[(trifluoroboranuidyl) methyl]carbamate (2.00 g, 8.416 mmol, 2 equiv) in Dioxane (15 mL) and water (3 mL) were added Cs₂CO₃ (4.11 g, 12.624 mmol, 3 equiv), Catacxium A (0.30 g, 0.842 mmol, 0.2 equiv) and Pd(OAc)₂ (0.43 g, 1.914 mmol, 0.2 equiv) in portions at room temperature under nitrogen atmosphere. The resulting mixture was stirred at 100° C. for 30 min under nitrogen atmosphere. The resulting mixture was filtered, the filter cake was washed with MeOH (3×5 mL). The filtrate was concentrated under reduced pressure. The residue was purified by reversed-phase flash chromatography with the following conditions: column, C18 silica gel; mobile phase, MeCN in Water (0.1% FA), 10% to 80% gradient in 10 min; detector, UV 254 nm. This resulted in tert-butyl N-({4-hydroxy-3-[(4-methylpiperazin-1-yl) methyl]phenyl}methyl) carbamate (1 g, 70.85% yield) as a white solid.

Step 3: A solution of tert-butyl N-({4-hydroxy-3-[(4-methylpiperazin-1-yl) methyl]phenyl}methyl) carbamate (1 g, 2.985 mmol, 1 equiv) in DCM (9 mL) in HCl in 1,4-dioxane (4.0 M) (3 mL) was stirred at room temperature for 1 h under. The resulting mixture was concentrated under reduced pressure to afford 4-(aminomethyl)-2-[(4-methylpiperazin-1-yl) methyl]phenol hydrochloride (750 mg, 92.57% yield) as a white solid. The crude resulting mixture was used in the next step directly without further purification.

Step 4: A solution of 4-(aminomethyl)-2-[(4-methylpiperazin-1-yl) methyl]phenol (700 mg, 2.975 mmol, 1 equiv) in Dioxane (12 mL) and 50% NaHCO₃ aqueous solution (4 mL). To the above mixture was added methyl 2,5-dioxopyrrole-1-carboxylate (692.07 mg, 4.463 mmol, 1.5 equiv) in portions at 0° C. The resulting mixture was stirred at 0° C. for additional 30 min. The resulting mixture was extracted with CH₂Cl₂ (3×10 mL). The combined organic layers were washed with water (3×10 mL), dried over anhydrous Na₂SO₄. After filtration, the filtrate was concentrated under reduced pressure. The residue was purified by reversed-phase flash chromatography with the following conditions: column, C18 silica gel; mobile phase, MeCN in Water (0.1% FA), 10% to 95% gradient in 10 min; detector, UV 254 nm. This resulted in 1-({4-hydroxy-3-[(4-methylpiperazin-1-yl) methyl]phenyl}methyl) pyrrole-2,5-dione (500 mg, 53.30% yield) as a white solid.

Step 5: To a stirred mixture of 1-({4-hydroxy-3-[(4-methylpiperazin-1-yl) methyl]phenyl}methyl) pyrrole-2,5-dione (200 mg, 0.634 mmol, 1 equiv) and 3-(fluorosulfonyl)-1,2-dimethylimidazol-1-ium triflate (249.80 mg, 0.761 mmol, 1.2 equiv) in DCM (5 mL) was added TEA (96.26 mg, 0.951 mmol, 1.5 equiv) in portions at 0° C. The resulting mixture was stirred at 0° C. for additional 30 min. The resulting mixture was filtered, the filter cake was washed with water (3×5 mL). The filtrate was concentrated under reduced pressure. The residue was purified by reversed-phase flash chromatography with the following conditions: column, C18 silica gel; mobile phase, MeCN in Water (0.1% FA), 10% to 95% gradient in 10 min; detector, UV 254 nm. This resulted in 4-[(2,5-dioxopyrrol-1-yl) methyl]-2-[(4-methylpiperazin-1-yl) methyl]phenyl sulfurofluoridate (100 mg, 39.68% yield, 96.3% purity) as a light yellow oil. LCMS:(ES, m/z): [M+H]⁺=398.1.

¹H NMR (400 MHz, DMSO-d₆, ppm) δ 8.15 (d, J=1.7 Hz, 1H), 7.49 (d, J=8.5 Hz, 1H), 7.43 (d, J=2.3 Hz, 1H), 7.33 (d, J=8.6 Hz, 1H), 7.11 (d, J=1.5 Hz, 2H), 4.65 (s, 2H), 3.56 (s, 2H), 2.36 (s, 8H), 2.18 (d, J=1.7 Hz, 3H).

### Example 95. Synthesis of (S)-4-(1-(2,5-dioxo-2,5-dihydro-1H-pyrrol-1-yl)ethyl)phenyl sulfurofluoridate (MFS-5-8)

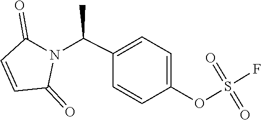 [SMILES: C[C@@H](c1ccc(OS(=O)(=O)F)cc1)N1C(=O)C=CC1=O]

Step 1: A solution of 4-[(1S)-1-aminoethyl]phenol (1 g, 7.290 mmol, 1 equiv) in Dioxane (9 mL) and 50% NaHCO3 aqueous solution (3 mL). To the above mixture was added methyl 2,5-dioxopyrrole-1-carboxylate (1.36 g, 8.748 mmol, 1.2 equiv) in portions at 0° C. The resulting mixture was stirred at 0° C. for additional 30 min. The resulting mixture was extracted with CH2Cl2 (3×10 mL). The combined organic layers were washed with water (3×10 mL), dried over anhydrous Na2SO4. After filtration, the filtrate was concentrated under reduced pressure. The residue was purified by reversed-phase flash chromatography with the following conditions: column, C18 silica gel; mobile phase, MeCN in Water (0.1% FA), 10% to 80% gradient in 10 min; detector, UV 254 nm. This resulted in 1-[(1S)-1-(4-hydroxyphenyl)ethyl]pyrrole-2,5-dione (500 mg, 31.58% yield) as a white solid.

Step 2: To a stirred solution of (S)-1-(1-(4-hydroxyphenyl)ethyl)-1H-pyrrole-2,5-dione (200 mg, 0.634 mmol, 1 equiv) and 3-(fluorosulfonyl)-1,2-dimethylimidazol-1-ium triflate (249.80 mg, 0.761 mmol, 1.2 equiv) in MeCN (5 mL) were added TEA (96.26 mg, 0.951 mmol, 1.5 equiv) dropwise dropwise at 0° C. The resulting mixture was stirred at room temperature for additional 0.5 h. The resulting mixture was concentrated under vacuum. The residue was purified by reversed-phase flash chromatography with the following conditions: column, C18 silica gel; mobile phase, MeCN in Water (0.1% FA), 10% to 70% gradient in 10 min; detector, UV 254 nm. This resulted in (S)-4-(1-(2,5-dioxo-2,5-dihydro-1H-pyrrol-1-yl)ethyl)phenyl sulfurofluoridate (100 mg, 39.68% yield, 96.3% purity) as a light yellow oil. GCMS: GC-MS: (EI+, m/z, M⁺)=299.0.

¹H NMR (300 MHz, DMSO-d₆) δ 7.67-7.40 (m, 4H), 7.03 (s, 2H), 5.36-5.28 (m, 1H), 1.72 (d, J=7.2 Hz, 3H).

### Example 96. Synthesis of (R)-4-(1-(2,5-dioxo-2,5-dihydro-1H-pyrrol-1-yl)ethyl)phenyl sulfurofluoridate (MFS-5-9)

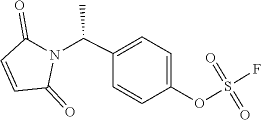 [SMILES: C[C@H](c1ccc(OS(=O)(=O)F)cc1)N1C(=O)C=CC1=O]

Step 1: A solution of (R)-4-(1-aminoethyl)phenol (1 g, 7.290 mmol, 1 equiv) in Dioxane (9 mL) and 50% NaHCO3 aqueous solution (3 mL). To the above mixture was added methyl 2,5-dioxopyrrole-1-carboxylate (1.36 g, 8.748 mmol, 1.2 equiv) in portions at 0° C. The resulting mixture was stirred at 0° C. for additional 30 min. The resulting mixture was extracted with CH2Cl2 (3×10 mL). The combined organic layers were washed with water (3×10 mL), dried over anhydrous Na2SO4. After filtration, the filtrate was concentrated under reduced pressure. The residue was purified by reversed-phase flash chromatography with the following conditions: column, C18 silica gel; mobile phase, MeCN in Water (0.1% FA), 10% to 50% gradient in 10 min; detector, UV 254 nm. This resulted in (R)-1-(1-(4-hydroxyphenyl)ethyl)-1H-pyrrole-2,5-dione (500 mg, 31.58% yield) as a white solid.

Step 2: To a stirred solution of (R)-1-(1-(4-hydroxyphenyl)ethyl)-1H-pyrrole-2,5-dione and 3-(fluorosulfonyl)-1,2-dimethylimidazol-1-ium triflate (249.80 mg, 0.761 mmol, 1.2 equiv) in MeCN (5 mL) were added TEA (96.26 mg, 0.951 mmol, 1.5 equiv) dropwise dropwise at 0° C. The resulting mixture was stirred at room temperature for additional 0.5 h. The resulting mixture was concentrated under vacuum. The residue was purified by reversed-phase flash chromatography with the following conditions: column, C18 silica gel; mobile phase, MeCN in Water (0.1% FA), 10% to 50% gradient in 10 min; detector, UV 254 nm. This resulted in (R)-4-(1-(2,5-dioxo-2,5-dihydro-1H-pyrrol-1-yl)ethyl)phenyl sulfurofluoridate (100 mg, 39.68% yield, 100.0% purity) as a light yellow oil. GCMS: GC-MS: (EI+, m/z, M⁺)=299.0.

¹H NMR (300 MHz, DMSO-d₆) δ 7.56 (t, J=7.0 Hz, 4H), 7.03 (s, 2H), 5.35-5.28 (m, 1H), 1.72 (d, J=7.2 Hz, 3H).

### Example 97. Synthesis of 3-carbamoyl-4-((2,5-dioxo-2,5-dihydro-1H-pyrrol-1-yl)methyl)phenyl sulfurofluoridate (MFS-5-10)

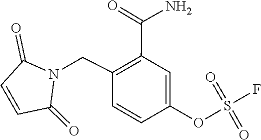 [SMILES: NC(=O)c1cc(OS(=O)(=O)F)ccc1CN1C(=O)C=CC1=O]

Step 1: A solution of potassium tert-butyl N-[(trifluoroboranuidyl) methyl]carbamate (2.67 g, 11.26 mmol, 1.30 equiv), methyl 2-bromo-5-hydroxybenzoate (2 g, 8.65 mmol, 1.00 equiv), di(1-adamantyl)-N-butylphosphine (0.62 g, 1.73 mmol, 0.2 equiv), Pd(OAc)₂ (0.19 g, 0.86 mmol, 0.1 equiv), Dioxane (40 mL) and Cs₂CO₃ (8.46 g, 25.96 mmol, 3 equiv) in Dioxane (40 mL) was stirred at 100° C. for 1 h under nitrogen atmosphere. The mixture was allowed to cool down to room temperature. The resulting mixture was filtered, the filter cake was washed with MeCN (3×5 mL). The filtrate was concentrated under reduced pressure. The residue was purified by reversed-phase flash chromatography with the following conditions: column, C18 silica gel; mobile phase, MeCN in Water (0.1% FA), 10% to 50% gradient in 10 min; detector, UV 254 nm. This resulted in methyl 2-{[(tert-butoxycarbonyl) amino]methyl}-5-hydroxybenzoate (1.5 g, 61.60% yield, 95% purity) as a yellow oil.

Step 2: A solution of methyl 2-{[(tert-butoxycarbonyl) amino]methyl}-5-hydroxybenzoate (1.5 g, 5.33 mmol, 1 equiv) in ammonium hydroxide (15 mL) was stirred at 80° C. for 30 min under nitrogen atmosphere. The residue was purified by reversed-phase flash chromatography with the following conditions: column, C18 silica gel; mobile phase, MeCN in Water (0.1% NH₃·H₂O), 10% to 50% gradient in 10 min; detector, UV 254 nm. This resulted in tert-butyl N-[(2-carbamoyl-4-hydroxyphenyl) methyl]carbamate (400 mg, 28.17% yield, 95% purity) as a yellow oil.

Step 3: A solution of tert-butyl N-[(2-carbamoyl-4-hydroxyphenyl) methyl]carbamate (400 mg, 1.502 mmol, 1 equiv) and HCl in 1,4-dioxane (4.0 M) (5 mL) in DCM (5 mL) was stirred at 50° C. for 2 h under nitrogen atmosphere. The resulting mixture was concentrated under reduced pressure. The crude product was used in the next step directly without further purification.

Step 4: A solution of methyl 2,5-dioxopyrrole-1-carboxylate (336.02 mg, 2.16 mmol, 1.2 equiv), NaHCO₃ (600 mg, 7.14 mmol, 3.96 equiv) and 2-(aminomethyl)-5-hydroxybenzamide (300 mg, 1.80 mmol, 1.00 equiv), H₂O (6 mL) in Dioxane (24 mL) was stirred at room temperature for 1 h under nitrogen atmosphere. The resulting mixture was concentrated under reduced pressure. The residue was purified by reversed-phase flash chromatography with the following conditions: column, C18 silica gel; mobile phase, MeCN in Water (0.1% FA), 10% to 50% gradient in 10 min; detector, UV 254 nm. This resulted in 2-[(2,5-dioxopyrrol-1-yl) methyl]-5-hydroxybenzamide (200 mg, 44.99% yield, 95% purity) as a yellow oil.

Step 5: A solution of 2-[(2,5-dioxopyrrol-1-yl)methyl]-5-hydroxybenzamide (200 mg, 0.81 mmol, 1 equiv), 3-(fluorosulfonyl)-1,2-dimethylimidazol-1-ium triflate (346.62 mg, 1.05 mmol, 1.3 equiv) and Et₃N (246.59 mg, 2.43 mmol, 3 equiv) in MeCN (5 mL) was stirred at 0° C. for 30 min under nitrogen atmosphere. The residue was purified by reversed-phase flash chromatography with the following conditions: column, C18 silica gel; mobile phase, MeCN in Water (0.1% FA), 10% to 50% gradient in 10 min; detector, UV 254 nm. This resulted in 3-carbamoyl-4-[(2,5-dioxopyrrol-1-yl) methyl]phenyl sulfurofluoridate (82.5 mg, 30.94% yield, 98.9% purity) as a white solid. LCMS:(ES, m/z): [M+H]⁺=329.1.

¹H NMR (300 MHz, DMSO-d₆, ppm) δ 8.11 (s, 1H), 7.75 (s, 1H), 7.70-7.57 (m, 2H), 7.31 (d, J=8.6 Hz, 1H), 7.12 (s, 2H), 4.81 (s, 2H).

### Example 98. Synthesis of (S)-5-(2,5-dioxo-2,5-dihydro-1H-pyrrol-1-yl)-5,6,7,8-tetrahydronaphthalen-2-yl sulfurofluoridate (MFS-5-13)

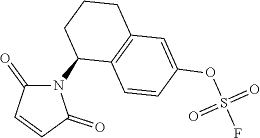

Step 1: A solution of 5-amino-5,6,7,8-tetrahydronaphthalen-2-ol (600 mg, 3.676 mmol, 1 equiv) in 1,4-dioxane (12.0 mL) and 50% NaHCO₃ (4.0 mL) was treated with methyl 2,5-dioxopyrrole-1-carboxylate (684.22 mg, 4.411 mmol, 1.20 equiv) at 0° C. The resulting mixture was stirred at 25° C. for additional 2 h. The reaction was monitored by LCMS. The reaction was quenched with saturated aqueous solution of citric acid at 0° C. after the reaction is completed. The resulting mixture was concentrated under vacuum. The residue was purified by reversed-phase flash chromatography with the following conditions: column, C18 silica gel; mobile phase, MeCN in Water (0.1% FA), 10% to 80% gradient in 12 min; detector, UV 254 nm. This resulted in 1-(6-hydroxy-1,2,3,4-tetrahydronaphthalen-1-yl) pyrrole-2,5-dione (400 mg, 44.73% yield) as a white solid.

Step 2: To a stirred mixture of 1-(6-hydroxy-1,2,3,4-tetrahydronaphthalen-1-yl)pyrrole-2,5-dione (400 mg, 1.664 mmol, 1 equiv) and 3-(fluorosulfonyl)-1,2-dimethylimidazol-1-ium triflate (647.70 mg, 1.973 mmol, 1.2 equiv) in MeCN (8.0 mL) was added TEA (332.79 mg, 3.289 mmol, 2 equiv) dropwise at 0° C. The resulting mixture was stirred at 25° C. for additional 30 min. The reaction was monitored by LCMS. The resulting mixture was concentrated under vacuum. The residue was purified by reversed-phase flash chromatography with the following conditions: column, C18 silica gel; mobile phase, MeCN in Water (0.1% FA), 10% to 50% gradient in 10 min; detector, UV 254 nm. This resulted in 5-(2,5-dioxopyrrol-1-yl)-5,6,7,8-tetrahydronaphthalen-2-yl sulfurofluoridate (180 mg, 33.65% yield) as a white solid. The crude product was purified by chiral HPLC. CHIRALPAK IJ-3, n-Hexane (0.1% DEA)/EtOH, flow rate=1.0000 mL/min, λ=230 nm, retention time: 1.422 (MFS-5-13-0), 1.945. GCMS: (ES, m/z): [M]⁺=325.0.

¹H NMR (300 MHz, DMSO-d₆, ppm) δ 7.37 (s, 1H), 7.27 (dd, J=8.7, 2.6 Hz, 1H), 7.14 (d, J=8.7 Hz, 1H), 7.07 (s, 2H), 5.19 (dd, J=10.7, 5.5 Hz, 1H), 2.90-2.79 (m, 2H), 2.19-2.04 (m, 1H), 2.03-1.89 (m, 2H), 1.88-1.69 (m, 1H).

### Example 99. Synthesis of (R)-5-(2,5-dioxo-2,5-dihydro-1H-pyrrol-1-yl)-5,6,7,8-tetrahydronaphthalen-2-yl sulfurofluoridate (MFS-5-14)

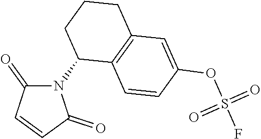

Step 1: A solution of (5R)-5-amino-5,6,7,8-tetrahydronaphthalen-2-ol (500 mg, 3.063 mmol, 1 equiv) in 1,4-dioxane (9 mL) and 50% NaHCO₃ (3 mL) was treated with methyl 2,5-dioxopyrrole-1-carboxylate (570.18 mg, 3.676 mmol, 1.20 equiv) at 0° C. The resulting mixture was stirred at 25° C. for additional 2 h. The reaction was monitored by LCMS. The reaction was quenched with saturated aqueous solution of citric acid at 0° C. after the reaction is completed. The resulting mixture was concentrated under vacuum. The residue was purified by reversed-phase flash chromatography with the following conditions: column, C18 silica gel; mobile phase, MeCN in Water (0.1% FA), 10% to 80% gradient in 12 min; detector, UV 254 nm. This resulted in 1-[(1R)-6-hydroxy-1,2,3,4-tetrahydronaphthalen-1-yl]pyrrole-2,5-dione (240 mg, 32.21% yield) as a colorless oil.

Step 2: To a stirred mixture of 1-[(1R)-6-hydroxy-1,2,3,4-tetrahydronaphthalen-1-yl]pyrrole-2,5-dione (200 mg, 0.822 mmol, 1.0 equiv) and 3-(fluorosulfonyl)-1,2-dimethylimidazol-1-ium triflate (323.85 mg, 0.986 mmol, 1.2 equiv) in MeCN (5.0 mL) was added TEA (166.39 mg, 1.644 mmol, 2.0 equiv) dropwise at 0° C. The resulting mixture was stirred at 25° C. for additional 30 min. The reaction was monitored by LCMS. The resulting mixture was concentrated under vacuum. The residue was purified by reversed-phase flash chromatography with the following conditions: column, C18 silica gel; mobile phase, MeCN in Water (0.1% FA), 10% to 50% gradient in 10 min; detector, UV 254 nm. This resulted in (5R)-5-(2,5-dioxopyrrol-1-yl)-5,6,7,8-tetrahydronaphthalen-2-yl sulfurofluoridate (55 mg, 20.56% yield) as a white solid. GCMS: (ES, m/z): [M]⁺=324.9.

¹H NMR (300 MHz, DMSO-d₆, ppm) δ 7.38 (d, J=1.9 Hz, 1H), 7.27 (dd, J=8.7, 2.7 Hz, 1H), 7.14 (d, J=8.6 Hz, 1H), 7.07 (s, 2H), 5.19 (dd, J=10.7, 5.5 Hz, 1H), 2.93-2.77 (m, 2H), 2.16-2.04 (m, 1H), 2.03-1.89 (m, 2H), 1.88-1.72 (m, 1H).

### Example 100. Synthesis of 3-(4-((2,5-dioxo-2,5-dihydro-1H-pyrrol-1-yl)methyl)piperidin-1-yl)phenyl sulfurofluoridate (MFS-8-1)

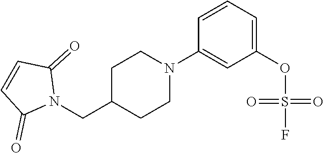 [SMILES: O=C1C=CC(=O)N1CC1CCN(c2cccc(OS(=O)(=O)F)c2)CC1]

Step 1: To a stirred solution of tert-butyl N-(piperidin-4-ylmethyl) carbamate (3 g, 13.998 mmol, 1 equiv), 1-(benzyloxy)-3-bromobenzene (4.42 g, 16.798 mmol, 1.2 equiv), t-BuONa (4.04 g, 41.994 mmol, 3 equiv), XPhos (1.33 g, 2.800 mmol, 0.2 equiv) and Pd2(dba)3 (1.28 g, 1.400 mmol, 0.1 equiv) in toluene (50 mL) at room temperature under nitrogen atmosphere. The resulting mixture was stirred at 100° C. for additional 3 h. The mixture was allowed to cool down to room temperature. The resulting mixture was diluted with 50 mL H2O and extracted with EtOAc (50 mL×3). The combined organic layers were washed with brine (50 mL) dried over anhydrous Na2SO4. After filtration, the filtrate was concentrated under reduced pressure. The residue was purified by silica gel column chromatography, eluted with PE/THF (3/1) to afford tert-butyl N-({1-[3-(benzyloxy) phenyl]piperidin-4-yl}methyl)carbamate (1.7 g, 30.63% yield) as a yellow oil.

Step 2: To a stirred solution of tert-butyl N-({1-[3-(benzyloxy) phenyl]piperidin-4-yl}methyl) carbamate (1.7 g, 4.287 mmol, 1 equiv) and Pd/C (0.23 g, 2.161 mmol, 0.50 equiv) in MeOH (15 mL) was room temperature under hydrogen atmosphere. The resulting mixture was stirred at room temperature for additional 2 h under hydrogen atmosphere. The resulting mixture was filtered, the filter cake was washed with methanol (5 mL×3). The filtrate was concentrated under reduced pressure. The filtrate was concentrated under reduced pressure to afford tert-butyl N-{[1-(3-hydroxyphenyl) piperidin-4-yl]methyl}carbamate (1.5 g, 114.19% yield) as a light yellow oil. The crude product was used in the next step directly without further purification.

Step 3: To a stirred solution of tert-butyl N-{[1-(3-hydroxyphenyl) piperidin-4-yl]methyl}carbamate (1.5 g, 4.895 mmol, 1 equiv) and 3-(fluorosulfonyl)-1,2-dimethylimidazol-1-ium triflate (2.41 g, 7.342 mmol, 1.5 equiv) in MeCN (20 mL) were added TEA (0.99 g, 9.790 mmol, 2 equiv) dropwise at 0° C. under nitrogen atmosphere. The resulting mixture was stirred at room temperature for additional 30 min. The resulting mixture was concentrated under reduced pressure. The residue was purified by silica gel column chromatography, eluted with PE /THF (1:1) to afford tert-butyl N-[(1-{3-[(fluorosulfonyl)oxy]phenyl}piperidin-4-yl) methyl]carbamate (800 mg, 42.07% yield) as a light yellow oil.

Step 4: To a stirred solution tert-butyl N-[(1-{3-[(fluorosulfonyl)oxy]phenyl}piperidin-4-yl) methyl]carbamate (800 mg, 2.059 mmol, 1 equiv) in DCM (4 mL) and HCl in 1,4-dioxane (4.0 M) (4 mL) at room temperature under nitrogen atmosphere. The resulting mixture was stirred at room temperature for additional 1 h. The precipitated solids were collected by filtration and washed with DCM (3 mL×3). This resulted in 3-[4-(aminomethyl) piperidin-1-yl]phenyl sulfurofluoridate (700 mg, 117.88% yield) as a light yellow solid.

Step 5: To a stirred solution of 3-[4-(aminomethyl) piperidin-1-yl]phenyl sulfurofluoridate (700 mg, 2.428 mmol, 1 equiv) and maleic anhydride (476.10 mg, 4.856 mmol, 2 equiv) in AcOH (10 mL) at room temperature under nitrogen atmosphere. The resulting mixture was stirred at 100° C. for additional overnight. The mixture was allowed to cool down to room temperature. The resulting mixture was concentrated under reduced pressure. The residue was purified by reversed-phase flash chromatography with the following conditions: column, C18 silica gel; mobile phase, MeCN in Water (0.1% FA), 10% to 80% gradient in 10 min; detector, UV 254 nm. This resulted in 3-{4-[(2,5-dioxopyrrol-1-yl) methyl]piperidin-1-yl}phenyl sulfurofluoridate (55.2 mg, 6.17% yield, 97.9% purity) as a light yellow oil. LCMS:(ES, m/z): [M+1]+=369.2.

¹H NMR (300 MHz, DMSO-d6) δ7.35 (t, J=8.2 Hz, 1H), 7.09-6.97 (m, 4H), 6.82 (d, J=8.1 Hz, 1H), 3.78 (d, J=12.8 Hz, 2H), 3.33 (m, 2H), 2.71 (td, J=12.5, 2.6 Hz, 2H), 1.79 (ddd, J=11.4, 7.6, 4.0 Hz, 1H), 1.63 (d, J=13.0 Hz, 2H), 1.27-1.15 (m, 2H).

### Example 101. Synthesis of 3-(4-(2,5-dioxo-2,5-dihydro-1H-pyrrol-1-yl)piperidine-1-carbonyl)phenyl sulfurofluoridate (MFS-8-4)

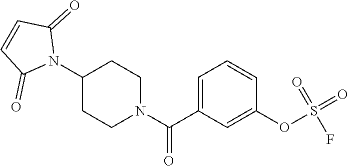 [SMILES: O=C(c1cccc(OS(=O)(=O)F)c1)N1CCC(N2C(=O)C=CC2=O)CC1]

Step 1: To a stirred mixture of tert-butyl 4-aminopiperidine-1-carboxylate (1 g, 4.993 mmol, 1 equiv) in NaHCO₃ aqueous solution (20 mL) was added methyl 2,5-dioxopyrrole-1-carboxylate (1.16 g, 7.490 mmol, 1.5 equiv) dropwise at 0° C. The resulting mixture was extracted with CH2Cl2 (3×20 mL). The combined organic layers were washed with water (3×20 mL), dried over anhydrous Na2SO4. After filtration, the filtrate was concentrated under reduced pressure. The residue was purified by reversed-phase flash chromatography with the following conditions: column, C18 silica gel; mobile phase, MeCN in Water (0.1% FA), 10% to 80% gradient in 10 min; detector, UV 254 nm. This resulted in tert-butyl 4-(2,5-dioxopyrrol-1-yl)piperidine-1-carboxylate (650 mg, 46.44% yield) as a white solid.

Step 2: A mixture of tert-butyl N-{2-[2-(2,5-dioxopyrrol-1-yl)ethoxy]ethyl}carbamate (500 mg, 1.759 mmol, 1 equiv) and phenylsulfonic acid (333.80 mg, 2.111 mmol, 1.2 equiv) in ACN (10 mL) was stirred at room temperature for 1 h. This resulted in 1-(piperidin-4-yl)pyrrole-2,5-dione (300 mg, 93.33% yield) as a white solid.. The crude product mixture was used in the next step directly without further purification.

Step 3: To a stirred mixture of 3-[(fluorosulfonyl)oxy]benzoic acid (439.83 mg, 1.998 mmol, 1.2 equiv) and DIEA (645.49 mg, 4.995 mmol, 3 equiv) and HATU (949.50 mg, 2.498 mmol, 1.5 equiv) in DCM (5 mL) was stirred at room temperature for 30 min. To the above mixture was added 1-(piperidin-4-yl) pyrrole-2,5-dione (300 mg, 1.665 mmol, 1 equiv) dropwise over 1 h at room temperature. The resulting mixture was extracted with CH2Cl2 (3×5 mL). The combined organic layers were washed with water (3×5 mL), dried over anhydrous Na2SO4. After filtration, the filtrate was concentrated under reduced pressure. The residue was purified by reversed-phase flash chromatography with the following conditions: column, C18 silica gel; mobile phase, MeCN in Water (0.1% FA), 10% to 95% gradient in 15 min; detector, UV 254 nm. This resulted in 3-[4-(2,5-dioxopyrrol-1-yl)piperidine-1-carbonyl]phenyl sulfurofluoridate (100 mg, 15.71% yield, 99.1% purity) as a white solid. LCMS: (ES, m/z): [M+H]⁺=383.0.

¹H NMR (300 MHz, DMSO-d₆) δ 7.77-7.64 (m, 3H), 7.61-7.51 (m, 1H), 7.00 (s, 2H), 4.57 (s, 1H), 4.12 (ddt, J=12.1, 8.0, 4.0 Hz, 1H), 3.55 (m, 1H), 3.20 (m, 1H), 2.87 (m, 1H), 2.08 (m, 2H), 1.69 (m, J=38.3 Hz, 2H).

### Example 102. Synthesis of 3-((3-(2,5-dioxo-2,5-dihydro-1H-pyrrol-1-yl)propyl)carbamoyl)phenyl sulfurofluoridate (MFS-8-5)

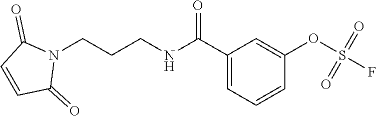 [SMILES: O=C(NCCCN1C(=O)C=CC1=O)c1cccc(OS(=O)(=O)F)c1]

Step 1: To a stirred mixture of tert-butyl N-(3-hydroxypropyl)carbamate (2 g, 11.414 mmol, 1 equiv) and maleimide (1.66 g, 17.121 mmol, 1.5 equiv) and PPh3 (5.99 g, 22.828 mmol, 2 equiv) in THF (40 mL) was added DIAD (6.92 g, 34.242 mmol, 3 equiv) dropwise at 0° C. under nitrogen atmosphere. The resulting mixture was extracted with CH2Cl2 (3×40 mL). The combined organic layers were washed with water (3×40 mL), dried over anhydrous Na2SO4. After filtration, the filtrate was concentrated under reduced pressure. The residue was purified by reversed-phase flash chromatography with the following conditions: column, C18 silica gel; mobile phase, MeCN in Water (0.1% FA), 10% to 50% gradient in 10 min; detector, UV 254 nm. This resulted in tert-butyl N-[3-(2,5-dioxopyrrol-1-yl)propyl]carbamate (1.5 g, 51.68% yield) as a yellow solid.

Step 2: A mixture of tert-butyl N-[3-(2,5-dioxopyrrol-1-yl) propyl]carbamate (500 mg, 1.966 mmol, 1 equiv) and phenylsulfonic acid (333.80 mg, 2.111 mmol, 1.2 equiv) in ACN (10 mL) was stirred at room temperature for 1 h. The resulting mixture was concentrated under reduced pressure to afford 1-(3-aminopropyl) pyrrole-2,5-dione (280 mg, 92.37% yield) as a white solid. The crude product mixture was used in the next step directly without further purification.

Step 3: To a stirred mixture of 3-[(fluorosulfonyl)oxy]benzoic acid (514.12 mg, 2.335 mmol, 1.2 equiv) and DIEA (754.51 mg, 5.838 mmol, 3 equiv) and HATU (887.89 mg, 2.335 mmol, 1.2 equiv) in DCM (5 mL) was stirred at room temperature for 30 min. To the above mixture was added 1-(3-aminopropyl)pyrrole-2,5-dione (300 mg, 1.946 mmol, 1 equiv) dropwise over. The resulting mixture was stirred at room temperature for additional 1 h. The resulting mixture was extracted with CH2Cl2 (3×5 mL). The combined organic layers were washed with water (3×5 mL), dried over anhydrous Na2SO4. After filtration, the filtrate was concentrated under reduced pressure. The residue was purified by reversed-phase flash chromatography with the following conditions: column, C18 silica gel; mobile phase, MeCN in Water (0.1% FA), 10% to 50% gradient in 10 min; detector, UV 254 nm. This resulted in 3-{[3-(2,5-dioxopyrrol-1-yl)propyl]carbamoyl}phenyl sulfurofluoridate (120 mg, 17.31% yield, 99.3% purity) as a white solid. LCMS:(ES, m/z): [M+H]⁺=357.1.

¹H NMR (300 MHz, DMSO-d₆) δ 8.70 (m, 1H), 8.00-7.97 (m, 2H), 7.84-7.76 (m, 1H), 7.72 (t, J=8.2 Hz, 1H), 7.02 (s, 2H), 3.48 (t, J=7.1 Hz, 2H), 3.29-3.23 m, 2H), 1.84-1.75 (m, 2H).

### Example 103. Synthesis of 4-(4-((2,5-dioxo-2,5-dihydro-1H-pyrrol-1-yl)methyl)piperidin-1-yl)phenyl sulfurofluoridate (MFS-9-1)

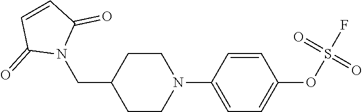 [SMILES: O=C1C=CC(=O)N1CC1CCN(c2ccc(OS(=O)(=O)F)cc2)CC1]

Step 1: To a stirred solution of tert-butyl N-[2-(piperidin-4-yl)ethyl]carbamate (2 g, 8.759 mmol, 1 equiv), 1-(benzyloxy)-3-bromobenzene (3.46 g, 13.139 mmol, 1.5 equiv), Pd₂(dba)₃ (0.80 g, 0.876 mmol, 0.1 equiv), XPhos (0.84 g, 1.752 mmol, 0.2 equiv) and t-BuONa (2.53 g, 26.277 mmol, 3 equiv) in toluene (20 mL) at room temperature under nitrogen atmosphere. The resulting mixture was stirred at 100° C. for additional overnight. The mixture was allowed to cool down to room temperature. The resulting mixture was diluted with 20 mL H₂O and extracted with EtOAc (20 mL×3). The combined organic layers were washed with brine (20 mL), dried over anhydrous Na₂SO₄. After filtration, the filtrate was concentrated under reduced pressure. The residue was purified by silica gel column chromatography, eluted with PE/THF (3:1) to afford tert-butyl N-(2-{1-[3-(benzyloxy) phenyl]piperidin-4-yl}ethyl)carbamate (1.9 g, 52.84% yield) as a light yellow solid.

Step 2: To a stirred solution of tert-butyl N-(2-{1-[3-(benzyloxy) phenyl]piperidin-4-yl}ethyl) carbamate (1.9 g, 4.628 mmol, 1 equiv) in DCM (10 mL) was added HCl in 1,4-dioxane (4.0 M) (10 mL) dropwise at room temperature under nitrogen atmosphere. The resulting mixture was stirred at room temperature for additional 2 h. The precipitated solids were collected by filtration and washed with diethyl ether (10 mL×3). This resulted in 2-{1-[3-(benzyloxy) phenyl]piperidin-4-yl}ethanamine (1.5 g, 104.41% yield) as a light yellow solid. The crude product was used in the next step directly without further purification.

Step 3: To a stirred solution of 2-{1-[3-(benzyloxy) phenyl]piperidin-4-yl}ethanamine (1.5 g, 4.832 mmol, 1 equiv) and palladium (0.3 g, 2.819 mmol, 0.58 equiv) in MeOH (15 mL) at room temperature under hydrogen atmosphere. The resulting mixture was stirred at room temperature for additional 2 h under hydrogen atmosphere. The resulting mixture was filtered, the filter cake was washed with MeOH (5 mL×3). The filtrate was concentrated under reduced pressure. This resulted in 3-[4-(2-aminoethyl) piperidin-1-yl]phenol (1.0 g, 93.94% yield) as a light yellow oil.

Step 4: To a stirred solution of 3-[4-(2-aminoethyl) piperidin-1-yl]phenol (1 g, 4.539 mmol, 1 equiv) and maleic anhydride (0.67 g, 6.808 mmol, 1.5 equiv) in MeCN (10 mL) were added TEA (1.38 g, 13.617 mmol, 3 equiv) dropwise at 0° C. under nitrogen atmosphere. The resulting mixture was stirred at room temperature for additional 1 h. The resulting mixture was concentrated under reduced pressure. The residue was purified by reversed-phase flash chromatography with the following conditions: column, C18 silica gel; mobile phase, MeCN in Water (0.1% FA), 10% to 50% gradient in 10 min; detector, UV 254 nm. This resulted in (2Z)-3-({2-[1-(3-hydroxyphenyl) piperidin-4-yl]ethyl}carbamoyl) prop-2-enoic acid (600 mg, 41.52% yield) as a light yellow oil.

Step 5: To a stirred solution of (2Z)-3-({2-[1-(3-hydroxyphenyl) piperidin-4-yl]ethyl}carbamoyl) prop-2-enoic acid (600 mg, 1.885 mmol, 1 equiv) and maleic anhydride (277.19 mg, 2.828 mmol, 1.5 equiv) in AcOH (6 mL) at room temperature under nitrogen atmosphere. The resulting mixture was stirred at 100° C. for additional overnight. The mixture was allowed to cool down to room temperature. The resulting mixture was concentrated under reduced pressure. The residue was purified by reversed-phase flash chromatography with the following conditions: column, C18 silica gel; mobile phase, MeCN in Water (0.1% FA), 10% to 50% gradient in 10 min; detector, UV 254 nm. This resulted in 1-{2-[1-(3-hydroxyphenyl) piperidin-4-yl]ethyl}pyrrole-2,5-dione (200 mg, 35.33% yield) as a light yellow oil.

Step 6: To a stirred solution of 1-{2-[1-(3-hydroxyphenyl) piperidin-4-yl]ethyl}pyrrole-2,5-dione (200 mg, 0.666 mmol, 1 equiv) and 3-(fluorosulfonyl)-1,2-dimethylimidazol-1-ium triflate (327.86 mg, 0.999 mmol, 1.5 equiv) in MeCN (3 mL) were added TEA (134.76 mg, 1.332 mmol, 2 equiv) dropwise at 0° C. under nitrogen atmosphere. The resulting mixture was stirred at room temperature for additional 30 min. The resulting mixture was concentrated under reduced pressure. The residue was purified by reversed-phase flash chromatography with the following conditions: column, C18 silica gel; mobile phase, MeCN in Water (0.1% FA), 10% to 80% gradient in 10 min; detector, UV 254 nm. This resulted in 3-{4-[2-(2,5-dioxopyrrol-1-yl) ethyl]piperidin-1-yl}phenyl sulfurofluoridate (20.7 mg, 8.13% yield, 99% purity) as a light yellow oil. LCMS:(ES, m/z): [M+H]⁺=383.1.

¹H NMR (400 MHz, DMSO-d₆, ppm) δ 7.34 (t, J=8.2 Hz, 1H), 7.08-7.00 (m, 4H), 6.85-6.79 (m, 1H), 3.78 (d, J=12.6 Hz, 2H), 3.46 (t, J=7.1 Hz, 2H), 2.70 (t, J=11.8 Hz, 2H), 1.78 (d, J=12.8 Hz, 2H), 1.46 (q, J=6.9 Hz, 2H), 1.36 (d, J=1.4 Hz, 1H), 1.21 (p, J=10.9, 10.2 Hz, 2H).

### Example 104. Synthesis of 3-((4-(2,5-dioxo-2,5-dihydro-1H-pyrrol-1-yl)butyl)carbamoyl)phenyl sulfurofluoridate (MFS-9-5)

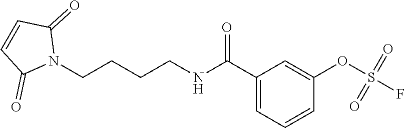 [SMILES: O=C(NCCCCN1C(=O)C=CC1=O)c1cccc(OS(=O)(=O)F)c1]

Step 1: To a stirred solution of tert-butyl N-(4-hydroxybutyl)carbamate (1 g, 5.284 mmol, 1 equiv), maleimide (0.62 g, 6.341 mmol, 1.2 equiv) and PPH3 (2.08 g, 7.926 mmol, 1.5 equiv) in THF (15 mL) were added DIAD (1.60 g, 7.926 mmol, 1.5 equiv) dropwise at 0° C. under nitrogen atmosphere. The resulting mixture was stirred at room temperature for additional 2 h. The resulting mixture was diluted with 15 mL H₂O and extracted with EtOAc (15 mL×3). The combined organic layers were washed with brine (15 mL), dried over anhydrous Na₂SO₄. The residue was purified by silica gel column chromatography, eluted with PE/EA (2:1) to afford tert-butyl N-[4-(2,5-dioxopyrrol-1-yl)butyl]carbamate (500 mg, 35.27% yield) as a light yellow oil.

Step 2: To a stirred solution of tert-butyl N-[4-(2,5-dioxopyrrol-1-yl)butyl]carbamate (500 mg, 1.863 mmol, 1 equiv) and benzenesulfonic acid (442.12 mg, 2.795 mmol, 1.5 equiv) in ACN (5 mL) at 0room temperature under nitrogen atmosphere. The resulting mixture was stirred at room temperature for additional 1 h. The precipitated solids were collected by filtration and washed with ACN (3 mL×3). This resulted in 1-(4-aminobutyl)pyrrole-2,5-dione (300 mg, 95.71% yield) as a white solid. The crude product was used in the next step directly without further purification.

Step 3: To a stirred solution of 3-hydroxybenzoic acid (5 g, 36.200 mmol, 1 equiv) and [(4-acetamidophenyl)(fluorosulfonyl)amino]sulfonyl fluoride (13.65 g, 43.440 mmol, 1.2 equiv) in THF (50 mL) were added DBU (12.12 g, 79.640 mmol, 2.2 equiv) dropwise at 0° C. under nitrogen atmosphere. The resulting mixture was stirred at room temperature for additional 2 h. The resulting mixture was diluted with 50 mL H2O and extracted with EtOAc (50 mL×3). The combined organic layers were washed with brine (50 mL), dried over anhydrous Na2SO4. The residue was purified by silica gel column chromatography, eluted with PE/THF (5:1) to afford 3-[(fluorosulfonyl)oxy]benzoic acid (2.2 g, 27.60% yield) as a light yellow solid.

Step 4: To a stirred solution of 1-(4-aminobutyl) pyrrole-2,5-dione (300 mg, 1.784 mmol, 1 equiv), HATU (1017.30 mg, 2.676 mmol, 1.5 equiv) and DIEA (345.79 mg, 2.676 mmol, 1.5 equiv) in DCM (5 mL) at room temperature under nitrogen atmosphere. The mixture was stirred for 15 min. 3-[(fluorosulfonyl)oxy]benzoic acid (471.24 mg, 2.141 mmol, 1.2 equiv) was added stirred for 30 min. The resulting mixture was diluted with 5 mL H2O and extracted with DCM (5 mL×3). The combined organic layers were washed with brine (5 mL), dried over anhydrous Na2SO4. The resulting mixture was concentrated under reduced pressure. The residue was purified by reversed-phase flash chromatography with the following conditions: column, C18 silica gel; mobile phase, MeCN in Water (0.1% FA), 10% to 80% gradient in 10 min; detector, UV 254 nm. This resulted in 3-{[4-(2,5-dioxopyrrol-1-yl)butyl]carbamoyl}phenyl sulfurofluoridate (300 mg, 45.42% yield, 98.8% purity) as a white solid. LCMS:(ES, m/z): [M+H]⁺=371.0.

¹H NMR (300 MHz, DMSO-d₆) δ 8.68 (t, J=5.6 Hz, 1H), 8.02-7.95 (m, 2H), 7.83-7.76 (m, 1H), 7.71 (t, J=8.2 Hz, 1H), 7.02 (s, 2H), 3.44 (t, J=6.5 Hz, 2H), 3.28 (q, J=6.3 Hz, 2H), 1.57-1.46 (m, 4H).

### Example 105. Synthesis of 3-((4-(2,5-dioxo-2,5-dihydro-1H-pyrrol-1-yl)cyclohexyl)carbamoyl)phenyl sulfurofluoridate (MFS-9-7)

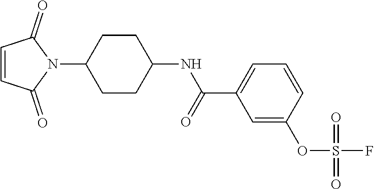 [SMILES: O=C(NC1CCC(N2C(=O)C=CC2=O)CC1)c1cccc(OS(=O)(=O)F)c1]

Step 1: To a stirred solution of tert-butyl N-(4-aminocyclohexyl)carbamate (1 g, 4.666 mmol, 1 equiv) in NaHCO₃(aq.) (10 mL) and 1,4-dioxane (30 mL) were added methyl 2,5-dioxopyrrole-1-carboxylate (0.87 g, 5.599 mmol, 1.2 equiv) in portions at 0° C. under nitrogen atmosphere. The resulting mixture was stirred at room temperature for additional 1 h. The resulting mixture was diluted with 20 mL H2O and extracted with EtoAC (20 mL×3). The combined organic layers were washed with brine (30 mL×1), dried over anhydrous Na2SO4. After filtration, the filtrate was concentrated under reduced pressure. The residue was purified by silica gel column chromatography, eluted with PE/THF (2:1) to afford tert-butyl N-[4-(2,5-dioxopyrrol-1-yl)cyclohexyl]carbamate (1 g, 36.40% yield) as a light yellow solid.

Step 2: To a stirred solution of tert-butyl N-[4-(2,5-dioxopyrrol-1-yl) cyclohexyl]carbamate (500 mg, 1.699 mmol, 1 equiv) and benzenesulfonic acid (322.41 mg, 2.039 mmol, 1.2 equiv) in MeCN (5 mL) at room temperature under nitrogen atmosphere. The resulting mixture was stirred at room temperature for additional 1 h. The precipitated solids were collected by filtration and washed with MeCN (3×3 mL). This resulted in 1-(4-aminocyclohexyl)pyrrole-2,5-dione (300 mg, 90.93% yield) as a white solid. The crude product/resulting mixture was used in the next step directly without further purification.

Step 3: To a stirred solution of 1-(4-aminocyclohexyl)pyrrole-2,5-dione (300 mg, 1.545 mmol, 1 equiv), HATU (1174.57 mg, 3.090 mmol, 2 equiv) and DIEA (399.25 mg, 3.090 mmol, 2 equiv) in DCM (5 mL) at room temperature under nitrogen atmosphere. The mixture was stirred for 15 min. 3-[(fluorosulfonyl)oxy]benzoic acid (510.09 mg, 2.317 mmol, 1.5 equiv) was added and stirred for 30 min. The resulting mixture was diluted with 5 mL H2O and extracted with DCM (5 mL×3). The combined organic layers were washed with brine (5 mL), dried over anhydrous Na2SO4. The resulting mixture was concentrated under reduced pressure. The residue was purified by reversed-phase flash chromatography with the following conditions: column, C18 silica gel; mobile phase, MeCN in Water (0.1% FA), 10% to 80% gradient in 10 min; detector, UV 254 nm. This resulted in 3-{[4-(2,5-dioxopyrrol-1-yl)cyclohexyl]carbamoyl}phenyl sulfurofluoridate (70 mg, 11.43% yield, 99.0% purity) as a white solid. LCMS:(ES, m/z): [M+H]⁺=397.0.

¹H NMR (300 MHz, DMSO-d₆) δ 8.42 (dd, J=58.3, 6.2 Hz, 1H), 8.02 (dq, J=6.2, 2.4, 1.9 Hz, 2H), 7.85-7.66 (m, 2H), 3.95-3.83 (m, 2H), 2.42-1.90 (m, 4H), 1.79-1.54 (m, 2H), 1.49-1.46 (m, 2H).

### Example 106. Synthesis of 3-(4-(2-(2,5-dioxo-2,5-dihydro-1H-pyrrol-1-yl)ethyl)piperidin-1-yl)phenyl sulfurofluoridate (MFS-9-2)

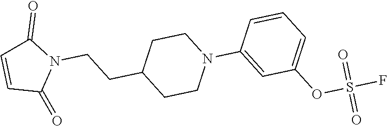 [SMILES: O=C1C=CC(=O)N1CCC1CCN(c2cccc(OS(=O)(=O)F)c2)CC1]

Step 1: To a stirred solution of tert-butyl N-[2-(piperidin-4-yl)ethyl]carbamate (2 g, 8.759 mmol, 1 equiv), 1-(benzyloxy)-3-bromobenzene (3.46 g, 13.139 mmol, 1.5 equiv), Pd₂(dba)₃ (0.80 g, 0.876 mmol, 0.1 equiv), XPhos (0.84 g, 1.752 mmol, 0.2 equiv) and t-BuONa (2.53 g, 26.277 mmol, 3 equiv) in toluene (20 mL) at room temperature under nitrogen atmosphere. The resulting mixture was stirred at 100° C. for additional overnight. The mixture was allowed to cool down to room temperature. The resulting mixture was diluted with 20 mL H₂O and extracted with EtOAc (20 mL×3). The combined organic layers were washed with brine (20 mL), dried over anhydrous Na₂SO₄. After filtration, the filtrate was concentrated under reduced pressure. The residue was purified by silica gel column chromatography, eluted with PE/THF (3:1) to afford tert-butyl N-(2-{1-[3-(benzyloxy) phenyl]piperidin-4-yl}ethyl)carbamate (1.9 g, 52.84% yield) as a light yellow solid.

Step 2: To a stirred solution of tert-butyl N-(2-{1-[3-(benzyloxy) phenyl]piperidin-4-yl}ethyl) carbamate (1.9 g, 4.628 mmol, 1 equiv) in DCM (10 mL) was added HCl in 1,4-dioxane (4.0 M) (10 mL) dropwise at room temperature under nitrogen atmosphere. The resulting mixture was stirred at room temperature for additional 2 h. The precipitated solids were collected by filtration and washed with diethyl ether (10 mL×3). This resulted in 2-{1-[3-(benzyloxy) phenyl]piperidin-4-yl}ethanamine (1.5 g, 104.41% yield) as a light yellow solid. The crude product was used in the next step directly without further purification.

Step 3: To a stirred solution of 2-{1-[3-(benzyloxy) phenyl]piperidin-4-yl}ethanamine (1.5 g, 4.832 mmol, 1 equiv) and palladium (0.3 g, 2.819 mmol, 0.58 equiv) in MeOH (15 mL) at room temperature under hydrogen atmosphere. The resulting mixture was stirred at room temperature for additional 2 h under hydrogen atmosphere. The resulting mixture was filtered, the filter cake was washed with MeOH (5 mL×3). The filtrate was concentrated under reduced pressure. This resulted in 3-[4-(2-aminoethyl) piperidin-1-yl]phenol (1.0 g, 93.94% yield) as a light yellow oil.

Step 4: To a stirred solution of 3-[4-(2-aminoethyl) piperidin-1-yl]phenol (1 g, 4.539 mmol, 1 equiv) and maleic anhydride (0.67 g, 6.808 mmol, 1.5 equiv) in MeCN (10 mL) were added TEA (1.38 g, 13.617 mmol, 3 equiv) dropwise at 0° C. under nitrogen atmosphere. The resulting mixture was stirred at room temperature for additional 1 h. The resulting mixture was concentrated under reduced pressure. The residue was purified by reversed-phase flash chromatography with the following conditions: column, C18 silica gel; mobile phase, MeCN in Water (0.1% FA), 10% to 50% gradient in 10 min; detector, UV 254 nm. This resulted in (2Z)-3-({2-[1-(3-hydroxyphenyl) piperidin-4-yl]ethyl}carbamoyl) prop-2-enoic acid (600 mg, 41.52% yield) as a light yellow oil.

Step 5: To a stirred solution of (2Z)-3-({2-[1-(3-hydroxyphenyl) piperidin-4-yl]ethyl}carbamoyl) prop-2-enoic acid (600 mg, 1.885 mmol, 1 equiv) and maleic anhydride (277.19 mg, 2.828 mmol, 1.5 equiv) in AcOH (6 mL) at room temperature under nitrogen atmosphere. The resulting mixture was stirred at 100° C. for additional overnight. The mixture was allowed to cool down to room temperature. The resulting mixture was concentrated under reduced pressure. The residue was purified by reversed-phase flash chromatography with the following conditions: column, C18 silica gel; mobile phase, MeCN in Water (0.1% FA), 10% to 50% gradient in 10 min; detector, UV 254 nm. This resulted in 1-{2-[1-(3-hydroxyphenyl) piperidin-4-yl]ethyl}pyrrole-2,5-dione (200 mg, 35.33% yield) as a light yellow oil.

Step 6: To a stirred solution of 1-{2-[1-(3-hydroxyphenyl) piperidin-4-yl]ethyl}pyrrole-2,5-dione (200 mg, 0.666 mmol, 1 equiv) and 3-(fluorosulfonyl)-1,2-dimethylimidazol-1-ium triflate (327.86 mg, 0.999 mmol, 1.5 equiv) in MeCN (3 mL) were added TEA (134.76 mg, 1.332 mmol, 2 equiv) dropwise at 0° C. under nitrogen atmosphere. The resulting mixture was stirred at room temperature for additional 30 min. The resulting mixture was concentrated under reduced pressure. The residue was purified by reversed-phase flash chromatography with the following conditions: column, C18 silica gel; mobile phase, MeCN in Water (0.1% FA), 10% to 80% gradient in 10 min; detector, UV 254 nm. This resulted in 3-{4-[2-(2,5-dioxopyrrol-1-yl) ethyl]piperidin-1-yl}phenyl sulfurofluoridate (20.7 mg, 8.13% yield, 99% purity) as a light yellow oil. LCMS:(ES, m/z): [M+H]⁺=383.1.

¹H NMR (400 MHz, DMSO-d₆, ppm) δ 7.34 (t, J=8.2 Hz, 1H), 7.08-7.00 (m, 4H), 6.85-6.79 (m, 1H), 3.78 (d, J=12.6 Hz, 2H), 3.46 (t, J=7.1 Hz, 2H), 2.70 (t, J=11.8 Hz, 2H), 1.78 (d, J=12.8 Hz, 2H), 1.46 (q, J=6.9 Hz, 2H), 1.36 (d, J=1.4 Hz, 1H), 1.21 (p, J=10.9, 10.2 Hz, 2H).

### Example 107. Synthesis of 3-((2-(2-(2,5-dioxo-2,5-dihydro-1H-pyrrol-1-yl)ethoxy)ethyl)carbamoyl)phenyl sulfurofluoridate (MFS-10-5)

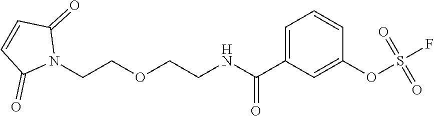 [SMILES: O=C(NCCOCCN1C(=O)C=CC1=O)c1cccc(OS(=O)(=O)F)c1]

A mixture of 3-[(fluorosulfonyl)oxy]benzoic acid (358.59 mg, 1.628 mmol, 1.2 equiv) and HATU (774.12 mg, 2.035 mmol, 1.5 equiv) and DIEA (526.26 mg, 4.071 mmol, 3 equiv) in DCM (5 mL) was stirred at room temperature for 30 min. To the above mixture was added 1-[2-(2-aminoethoxy)ethyl]pyrrole-2,5-dione (250 mg, 1.357 mmol, 1 equiv) dropwise over. The resulting mixture was stirred at room temperature for additional 1 h. The resulting mixture was extracted with CH2Cl2 (3×5 mL). The combined organic layers were washed with water (3×5 mL), dried over anhydrous Na₂SO₄. After filtration, the filtrate was concentrated under reduced pressure. The residue was purified by reversed-phase flash chromatography with the following conditions: column, C18 silica gel; mobile phase, MeCN in Water (0.1% FA), 10% to 50% gradient in 10 min; detector, UV 254 nm. This resulted in 3-({2-[2-(2,5-dioxopyrrol-1-yl)ethoxy]ethyl}carbamoyl)phenyl sulfurofluoridate (60 mg, 15.75% yield, 100.0% purity) as a white solid. LCMS: [M+H]⁺=387.1.

¹H NMR (300 MHz, DMSO-d₆) δ 8.70 (t, J=5.5 Hz, 1H), 8.00-7.97 (m, 2H), 7.85-7.76 (m, 1H), 7.71 (t, J=8.1 Hz, 1H), 6.97 (s, 2H), 3.62-3.49 (m, 6H), 3.41-3.35 (m, 2H).

### Example 108. Synthesis of 4-((2-(2,5-dioxo-2,5-dihydro-1H-pyrrol-1-yl)-7-azaspiro[3.5]nonan-7-yl)methyl)-2-fluorophenyl sulfurofluoridate (MFS-11-4)

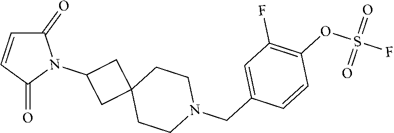 [SMILES: O=C1C=CC(=O)N1C1CC2(CCN(Cc3ccc(OS(=O)(=O)F)c(F)c3)CC2)C1]

Step 1: To a stirred solution of 2-fluoro-4-methylphenol (1 g, 7.928 mmol, 1 equiv) and 3-(fluorosulfonyl)-1,2-dimethylimidazol-1-ium triflate (3.90 g, 11.892 mmol, 1.5 equiv) in MeCN (15 mL) were added TEA (1.60 g, 15.856 mmol, 2 equiv) dropwise at 0° C. under nitrogen atmosphere. The resulting mixture was stirred at room temperature for additional 30 min. The resulting mixture was concentrated under reduced pressure. The residue was purified by reversed-phase flash chromatography with the following conditions: column, C18 silica gel; mobile phase, MeCN in Water (0.1% TFA), 10% to 90% gradient in 10 min; detector, UV 254 nm. This resulted in 2-fluoro-4-methylphenyl sulfurofluoridate (1 g, 60.59% yield) as a light yellow solid.

Step 2: To a stirred solution of 2-fluoro-4-methylphenyl sulfurofluoridate (1 g, 4.804 mmol, 1 equiv) and benzoyl benzenecarboperoxoate (0.12 g, 0.480 mmol, 0.1 equiv) in CCl₄ (10 mL) were added NBS (0.85 g, 4.804 mmol, 1 equiv) in portions at 0° C. under nitrogen atmosphere. The resulting mixture was stirred at 70° C. for additional 2 h. The mixture was allowed to cool down to room temperature. The resulting mixture was diluted with 20 mL H2O and extracted with DCM (20 mL×3). The combined organic layers were washed with brine (20 mL), dried over anhydrous Na2SO4. After filtration, the filtrate was concentrated under reduced pressure. The residue was purified by reversed-phase flash chromatography with the following conditions: column, C18 silica gel; mobile phase, MeCN in Water (0.1% FA), 10% to 90% gradient in 10 min; detector, UV 254 nm. This resulted in 4-(bromomethyl)-2-fluorophenyl sulfurofluoridate (500 mg, 36.26% yield) as a light yellow oil.

Step 3: To a stirred solution of 1-{7-azaspiro[3.5]nonan-2-yl}pyrrole-2,5-dione (300 mg, 1.362 mmol, 1 equiv) and 4-(bromomethyl)-2-fluorophenyl sulfurofluoridate (469.17 mg, 1.634 mmol, 1.2 equiv) in ACN (5 mL) were added TEA (275.64 mg, 2.724 mmol, 2 equiv) dropwise at 0° C. under nitrogen atmosphere. The resulting mixture was stirred at room temperature for additional 2 h. The resulting mixture was concentrated under reduced pressure. The residue was purified by reversed-phase flash chromatography with the following conditions: column, C18 silica gel; mobile phase, MeCN in Water (0.1% FA), 10% to 50% gradient in 10 min; detector, UV 254 nm. This resulted in 4-{[2-(2,5-dioxopyrrol-1-yl)-7-azaspiro[3.5]nonan-7-yl]methyl}-2-fluorophenyl sulfurofluoridate (120 mg, 20.66% yield, 98.8% purity) as a white solid. LCMS:(ES, m/z): [M+H]⁺=427.0.

¹H NMR (300 MHz, DMSO-d₆) δ 7.74 (t, J=8.0 Hz, 1H), 7.50 (dd, J=11.5, 1.9 Hz, 1H), 7.33 (d, J=8.5 Hz, 1H), 6.96 (s, 2H), 4.44 (p, J=9.0 Hz, 1H), 3.48 (s, 2H), 2.41-2.27 (m, 6H), 2.08-2.01 (m 2H), 1.62 (t, J=5.3 Hz, 4H).

### Example 109. Synthesis of 3-(2,5-dioxo-2,5-dihydro-1H-pyrrol-1-yl)benzenesulfonyl fluoride (MSF-3-2)

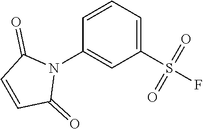 [SMILES: O=C1C=CC(=O)N1c1cccc(S(=O)(=O)F)c1]

Step 1: To a stirred solution of 3-nitrobenzenesulfonyl fluoride (2 g, 9.75 mmol, 1 equiv) in MeOH (20 ml) was added Pd/C (10% purity, 200 mg), the mixture was degassed and purged with hydrogen for several times, then stirred at 60° C. for 12 hours. The mixture was filtered, the filtrate was concentrated under reduced pressure in vacuum to afford 3-aminobenzenesulfonyl fluoride (1.5 g, 87.7% yield). as a yellow solid.

Step 2: To a stirred solution of 3-aminobenzenesulfonyl fluoride (1.5 g, 7.85 mmol, 1 equiv) and maleic anhydride (0.92 g, 9.42 mmol, 1.2 equiv) in HOAc (20 mL) at room temperature under nitrogen atmosphere. The resulting mixture was stirred at 100° C. for 16 h. The resulting mixture was concentrated under reduced pressure. The residue was purified by silica gel column chromatography, eluted with PE: EA (5:1) to afford 3-(2,5-dioxo-2,5-dihydro-1H-pyrrol-1-yl) benzenesulfonyl fluoride (400 mg, 18.34% yield, 99.9% purity) as alight yellow solid. GCMS:(ES, m/z): [M+H]⁺=255.0.

¹H NMR (300 MHz, DMSO-d₆) δ 8.32-8.10 (m, 2H), 8.10-7.86 (m, 2H), 7.28 (s, 2H).

### Example 110. Synthesis of 2-(methylsulfonyl)pyrimidin-4-yl sulfurofluoridate (PH-TOPAZ-FP-FS-2)

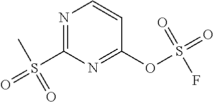

Step 1: To a stirred solution of 2-chloropyrimidin-4-ol (3 g, 22.98 mmol, 1.00 equiv) and BnBr (11.79 g, 68.95 mmol, 3.00 equiv) and Cs₂CO₃ (8.99 g, 27.58 mmol, 1.20 equiv) in DMF (50 mL) at room temperature under nitrogen atmosphere. The resulting mixture was stirred at room temperature for additional overnight. The resulting mixture was diluted with 50 mL H₂O and extracted with EtOAc (3×50 mL). The combined organic layer was washed with brine (25 mL), dried over anhydrous Na₂SO₄. After filtration, the filtrate was concentrated under reduced pressure. The residue was purified by silica gel column chromatography, eluted with PE/THF (3/1) to afford 4-(benzyloxy)-2-chloropyrimidine (2 g, 39.44% yield) as a light yellow solid.

Step 2: To a stirred solution of 4-(benzyloxy)-2-chloropyrimidine (1 g, 4.53 mmol, 1.00 equiv) and Sodium thiomethoxide (0.32 g, 4.53 mmol, 1.00 equiv) in DMF (10 mL) were at room temperature under nitrogen atmosphere. The resulting mixture was stirred at room temperature for additional 2 h. The resulting mixture was diluted with 10 mL H₂O and extracted with EtOAc (3×10 mL). The combined organic layer was washed with brine (10 mL), dried over anhydrous Na₂SO₄. After filtration, the filtrate was concentrated under reduced pressure. The residue was purified by silica gel column chromatography, eluted with PE/THF (4/1) to afford 4-(benzyloxy)-2-(methylsulfanyl) pyrimidine (600 mg, 56.99% yield) as a light yellow oil.

Step 3: To a stirred solution of 4-(benzyloxy)-2-(methylsulfanyl) pyrimidine (600 mg, 2.58 mmol, 1.00 equiv) in DCM (10 mL) were added boron tribromide (647.06 mg, 2.58 mmol, 1.00 equiv) dropwise at 0° C. under nitrogen atmosphere. The resulting mixture was stirred at room temperature for additional 3 h. The resulting mixture was concentrated under reduced pressure. The residue was purified by reversed-phase flash chromatography with the following conditions: column, C18 silica gel; mobile phase, MeCN in Water (0.1% FA), 10% to 95% gradient in 10 min; detector, UV 254 nm. This resulted in 2-(methylsulfanyl) pyrimidin-4-ol (250 mg, 68.08% yield) as a white solid.

Step 4: To a stirred solution of 2-(methylsulfanyl) pyrimidin-4-ol (250 mg, 1.76 mmol, 1.00 equiv) and 3-(fluorosulfonyl)-1,2-dimethylimidazol-1-ium triflate (1.15 g, 3.52 mmol, 2.00 equiv) in MeCN (5 mL) was added TEA (533.79 mg, 5.27 mmol, 3.00 equiv) dropwise at 0° C. The resulting mixture was stirred at room temperature for additional 1 h. The resulting mixture was concentrated under reduced pressure. The residue was purified by silica gel column chromatography, eluted with PE/THF (4/1) to afford 2-(methylsulfanyl) pyrimidin-4-yl sulfurofluoridate (120 mg, 30.44% yield) as a light yellow oil.

Step 5: To a stirred solution of 2-(methylsulfanyl) pyrimidin-4-yl sulfurofluoridate (120 mg, 0.54 mmol, 1.00 equiv) in DCM (3 mL) were added m-CPBA (184.70 mg, 1.07 mmol, 2.00 equiv) in portions at 0° C. under nitrogen atmosphere. The resulting mixture was stirred at 40° C. for additional 2 h. The mixture was allowed to cool down to room temperature. The resulting mixture was concentrated under reduced pressure. The residue was purified by reversed-phase flash chromatography with the following conditions: column, C18 silica gel; mobile phase, MeCN in Water (0.1% FA), 10% to 95% gradient in 10 min; detector, UV 254 nm. This resulted in 2-methanesulfonylpyrimidin-4-yl sulfurofluoridate (35 mg, 25.52% yield, 98.8% purity) as a light yellow solid.

¹H NMR (300 MHz, Chloroform-d, ppm): δ 9.16 (d, J=5.5 Hz, 1H), 7.44 (d, J=5.4 Hz, 1H), 3.43 (s, 3H).

### Example 111. Synthesis of 4-((4-(2,5-dioxo-2,5-dihydro-1H-pyrrol-1-yl)butyl)carbamoyl)phenyl sulfurofluoridate (Synthesis of MFS-10-4)

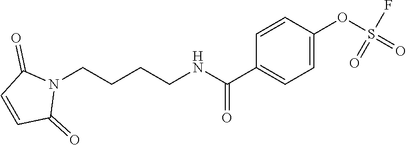 [SMILES: O=C(NCCCCN1C(=O)C=CC1=O)c1ccc(OS(=O)(=O)F)cc1]

A solution of 1-(4-aminobutyl) pyrrole-2,5-dione (150 mg, 0.89 mmol, 1 equiv), 4-[(fluorosulfonyl)oxy]benzoic acid (215.99 mg, 0.98 mmol, 1.1 equiv) and HATU (508.65 mg, 1.33 mmol, 1.5 equiv) in DCM (10 mL) was stirred at room temperature for 3 min under nitrogen atmosphere. To the above mixture was added DIEA (230.53 mg, 1.78 mmol, 2 equiv) dropwise over 2 min at 0° C. The resulting mixture was stirred at room temperature for additional 1 h. The residue was purified by reversed-phase flash chromatography with the following conditions: column, C18 silica gel; mobile phase, MeCN in Water (0.1% FA), 10% to 50% gradient in 10 min; detector, UV 254 nm. This resulted in 4-{[4-(2,5-dioxopyrrol-1-yl) butyl]carbamoyl}phenyl sulfurofluoridate (50.9 mg, 15.41% yield, 98.1% purity) as a white solid. LCMS:(ES, m/z): [M+H]⁺=371.0.

¹H NMR (300 MHz, DMSO-d₆, ppm) δ 8.63 (t, J=5.5 Hz, 1H), 8.06-7.95 (m, 2H), 7.76-7.67 (m, 2H), 7.02 (s, 2H), 3.43 (t, J=6.5 Hz, 2H), 3.27 (d, J=6.0 Hz, 2H), 1.51 (ddt, J=17.8, 14.3, 4.6 Hz, 4H).

### Example 112. Synthesis of 4-((4-(2,5-dioxo-2,5-dihydro-1H-pyrrol-1-yl)cyclohexyl)carbamoyl) phenyl sulfurofluoridate (MFS-10-6)

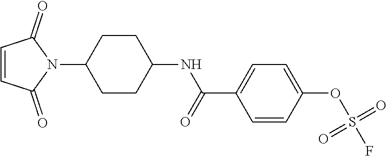 [SMILES: O=C(NC1CCC(N2C(=O)C=CC2=O)CC1)c1ccc(OS(=O)(=O)F)cc1]

Step 1: To a stirred solution of P-hydroxybenzoic acid (5 g, 36.200 mmol, 1 equiv) and [(4-acetamidophenyl)(fluorosulfonyl)amino]sulfonyl fluoride (13.65 g, 43.440 mmol, 1.2 equiv) in THF (50 mL) were added DBU (12.12 g, 79.640 mmol, 2.2 equiv) dropwise at 0° C. under nitrogen atmosphere. The resulting mixture was stirred at room temperature for additional 2 h. The resulting mixture was diluted with 50 mL H2O and extracted with EtOAc (50 mL×3). The combined organic layers were washed with brine (50 mL), dried over anhydrous Na2SO4. The residue was purified by silica gel column chromatography, eluted with PE/THF (5:1) to afford 4-[(fluorosulfonyl)oxy]benzoic acid (2 g, 25.09% yield) as a light yellow solid.

Step 2: To a stirred solution of 1-(4-aminocyclohexyl)pyrrole-2,5-dione (200 mg, 1.030 mmol, 1 equiv), HATU (587.28 mg, 1.545 mmol, 1.5 equiv) and DIEA (266.17 mg, 2.060 mmol, 2 equiv) in DCM (5 mL) at room temperature under nitrogen atmosphere. The mixture was stirred for 15 min. 4-[(fluorosulfonyl)oxy]benzoic acid (340.06 mg, 1.545 mmol, 1.5 equiv) was added stirred for 30 min. The resulting mixture was diluted with 5 mL H₂O and extracted with DCM (5 mL×3). The combined organic layers were washed with brine (5 mL), dried over anhydrous Na₂SO₄. The resulting mixture was concentrated under reduced pressure. The residue was purified by reversed-phase flash chromatography with the following conditions: column, C18 silica gel; mobile phase, MeCN in Water (0.1% FA), 10% to 80% gradient in 10 min; detector, UV 254 nm. This resulted in 4-{[4-(2,5-dioxopyrrol-1-yl) cyclohexyl]carbamoyl}phenyl sulfurofluoridate (80 mg, 19.60% yield, 100.0% purity) as a white solid. LCMS:(ES, m/z): [M+H]⁺=397.1.

¹H NMR (300 MHz, DMSO-d₆) δ 8.40 (dd, J=49.4, 6.2 Hz, 1H), 8.08-7.97 (m, 2H), 7.79-7.67 (m, 2H), 4.06-3.69 (m, 2H), 2.44-1.86 (m, 4H), 1.78-1.33 (m, 4H).

### Example 113. Synthesis of 5-((2-(2,5-dioxo-2,5-dihydro-1H-pyrrol-1-yl)-7-azaspiro[3.5]nonan-7-yl)methyl)-2-fluorophenyl sulfurofluoridate (MFS-10-7)

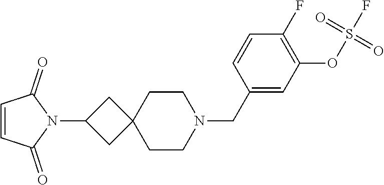 [SMILES: O=C1C=CC(=O)N1C1CC2(CCN(Cc3ccc(F)c(OS(=O)(=O)F)c3)CC2)C1]

Step 1: To a stirred solution of 2-fluoro-5-methylphenol (1 g, 7.928 mmol, 1 equiv) and 3-(fluorosulfonyl)-1,2-dimethylimidazol-1-ium triflate (3.90 g, 11.892 mmol, 1.5 equiv) in MeCN (15 mL) were added TEA (1.60 g, 15.856 mmol, 2 equiv) dropwise at 0° C. under nitrogen atmosphere. The resulting mixture was stirred at room temperature for additional 30 min. The resulting mixture was concentrated under reduced pressure. The residue was purified by reversed-phase flash chromatography with the following conditions: column, C18 silica gel; mobile phase, MeCN in Water (0.1% TFA), 10% to 90% gradient in 10 min; detector, UV 254 nm. This resulted in 2-fluoro-5-methylphenyl sulfurofluoridate (1 g, 60.59% yield) as a light yellow solid.

Step 2: To a stirred solution of 2-fluoro-5-methylphenyl sulfurofluoridate (1 g, 4.804 mmol, 1 equiv) and benzoyl benzenecarboperoxoate (0.12 g, 0.480 mmol, 0.1 equiv) in CCl₄ (10 mL) were added NBS (0.85 g, 4.804 mmol, 1 equiv) in portions at 0° C. under nitrogen atmosphere. The resulting mixture was stirred at 70° C. for additional 2 h. The mixture was allowed to cool down to room temperature. The resulting mixture was diluted with 20 mL H2O and extracted with DCM (20 mL×3). The combined organic layers were washed with brine (25 mL), dried over anhydrous Na2SO4. After filtration, the filtrate was concentrated under reduced pressure. The residue was purified by reversed-phase flash chromatography with the following conditions: column, C18 silica gel; mobile phase, MeCN in Water (0.1% FA), 10% to 90% gradient in 10 min; detector, UV 254 nm. This resulted in 5-(bromomethyl)-2-fluorophenyl sulfurofluoridate (500 mg, 36.26% yield) as a light yellow oil.

Step 3: To a stirred solution of 1-{7-azaspiro[3.5]nonan-2-yl}pyrrole-2,5-dione (300 mg, 1.362 mmol, 1 equiv) and 5-(bromomethyl)-2-fluorophenyl sulfurofluoridate (469.17 mg, 1.634 mmol, 1.2 equiv) in ACN (5 mL) were added TEA (275.64 mg, 2.724 mmol, 2 equiv) dropwise at 0° C. under nitrogen atmosphere. The resulting mixture was stirred at room temperature for additional 2 h. The resulting mixture was concentrated under reduced pressure. The residue was purified by reversed-phase flash chromatography with the following conditions: column, C18 silica gel; mobile phase, MeCN in Water (0.1% FA), 10% to 80% gradient in 10 min; detector, UV 254 nm. This resulted in 5-{[2-(2,5-dioxopyrrol-1-yl)-7-azaspiro[3.5]nonan-7-yl]methyl}-2-fluorophenyl sulfurofluoridate (90 mg, 15.50% yield, 100.0% purity) as a white solid. LCMS:(ES, m/z): [M+H]⁺=427.0.

¹H NMR (300 MHz, DMSO-d₆) δ 7.70 (d, J=7.4 Hz, 1H), 7.63-7.45 (m, 2H), 6.97 (s, 2H), 4.45 (p, J=8.9 Hz, 1H), 3.51 (s, 2H), 2.41-2.28 (m, 6H), 2.06 (t, J=10.3 Hz, 2H), 1.63 (s, 4H).

### Example 114. Synthesis of 3-(2-(2,5-dioxo-2,5-dihydro-1H-pyrrol-1-yl)-7-azaspiro[3.5]nonane-7-carbonyl)phenyl sulfurofluoridate (MFS-10-8)

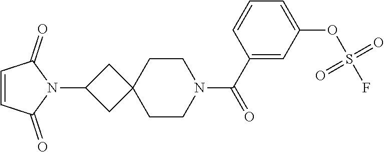 [SMILES: O=C(c1cccc(OS(=O)(=O)F)c1)N1CCC2(CC1)CC(N1C(=O)C=CC1=O)C2]

Step 1: To a stirred mixture of tert-butyl 2-amino-7-azaspiro[3.5]nonane-7-carboxylate (2 g, 2.083 mmol, 1 equiv) in NaHCO₃ aqueous solution (20 mL) was added methyl 2,5-dioxopyrrole-1-carboxylate (1.94 g, 3.125 mmol, 1.5 equiv) dropwise at 0° C. The resulting mixture was stirred at room temperature for 1 h. The resulting mixture was extracted with CH₂C12 (3×20 mL). The combined organic layers were washed with water (3×20 mL), dried over anhydrous Na₂SO₄. After filtration, the filtrate was concentrated under reduced pressure. The residue was purified by reversed-phase flash chromatography with the following conditions: column, C18 silica gel; mobile phase, MeCN in Water (0.1% FA), 10% to 80% gradient in 10 min; detector, UV 254 nm. This resulted in tert-butyl 2-(2,5-dioxocyclopent-3-en-1-yl)-7-azaspiro[3.5]nonane-7-carboxylate (1.8 g, 67.72% yield) as a white solid.

Step 2: A mixture of tert-butyl 2-(2,5-dioxocyclopent-3-en-1-yl)-7-azaspiro[3.5]nonane-7-carboxylate (450 mg, 1.409 mmol, 1 equiv) and phenylsulfonic acid (267.41 mg, 1.691 mmol, 1.2 equiv) in ACN (10 mL) was stirred at room temperature for 1 h. The resulting mixture was concentrated under reduced pressure. This resulted in 2-{7-azaspiro[3.5]nonan-2-yl}cyclopent-4-ene-1,3-dione (300 mg, 97.10% yield) as a white solid. The crude product mixture was used in the next step directly without further purification.

Step 3: To a stirred mixture of 3-[(fluorosulfonyl)oxy]benzoic acid (220.17 mg, 1.642 mmol, 1.2 equiv) and DIEA (530.46 mg, 4.104 mmol, 3 equiv) and HATU (780.29 mg, 2.052 mmol, 1.5 equiv) in DCM (5 mL) was stirred at room temperature for 30 min. To the above mixture was added 2-{7-azaspiro[3.5]nonan-2-yl}cyclopent-4-ene-1,3-dione (300 mg, 1.368 mmol, 1 equiv) dropwise over 1 h at room temperature. The resulting mixture was extracted with CH₂C12 (3×5 mL). The combined organic layers were washed with water (3×5 mL), dried over anhydrous Na₂SO₄. After filtration, the filtrate was concentrated under reduced pressure. The residue was purified by reversed-phase flash chromatography with the following conditions: column, C18 silica gel; mobile phase, MeCN in Water (0.1% FA), 10% to 95% gradient in 15 min; detector, UV 254 nm. This resulted in 3-[2-(2,5-dioxopyrrol-1-yl)-7-azaspiro[3.5]nonane-7-carbonyl]phenyl sulfurofluoridate (150 mg, 26.07% yield, 99.9% purity) as a white solid. LCMS: (ES, m/z): [M+H]⁺=423.1.

¹H NMR (300 MHz, DMSO-d₆, ppm) δ 7.73-7.63 (m, 3H), 7.55-7.52 (m, 1H), 6.98 (s, 2H), 4.49 (s, 1H), 3.57 (d, J=19.5 Hz, 2H), 3.22 (m, 2H), 2.44 (s, 2H), 2.15 (m, 2H), 1.65 (d, J=22.9 Hz, 4H).

### Example 115. Synthesis of 4-((2-(2,5-dioxo-2,5-dihydro-1H-pyrrol-1-yl)-7-azaspiro[3.5]nonan-7-yl)methyl)phenyl sulfurofluoridate (MFS-11-1)

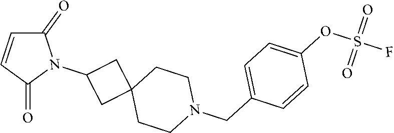 [SMILES: O=C1C=CC(=O)N1C1CC2(CCN(Cc3ccc(OS(=O)(=O)F)cc3)CC2)C1]

Step 1: To a stirred solution of tert-butyl N-{7-azaspiro [3.5]nonan-2-yl}carbamate (1.8 g, 7.489 mmol, 1 equiv) and 1-(benzyloxy)-4-(bromomethyl) benzene (2.49 g, 8.987 mmol, 1.2 equiv) in MeCN (20 mL) were added TEA (1.14 g, 11.233 mmol, 1.5 equiv) dropwise at 0° C. under nitrogen atmosphere. The resulting mixture was stirred at room temperature for additional 1 h. The resulting mixture was diluted with 20 mL H₂O and extracted with EtOAc (20 mL×3). The combined organic layers were washed with brine (20 mL), dried over anhydrous Na₂SO₄. After filtration, the filtrate was concentrated under reduced pressure. This resulted in tert-butyl N-(7-{[4-(benzyloxy) phenyl]methyl}-7-azaspiro [3.5]nonan-2-yl) carbamate (1.6 g, 48.93% yield) as a light yellow oil. The crude product was used in the next step directly without further purification.

Step 2: To a stirred solution of tert-butyl N-(7-{[4-(benzyloxy) phenyl]methyl}-7-azaspiro [3.5]nonan-2-yl)carbamate (1.6 g, 3.665 mmol, 1 equiv) in DCM (8 mL) was added tert-butyl N-(7-{[4-(benzyloxy)phenyl]methyl}-7-azaspiro[3.5]nonan-2-yl)carbamate (1.6 g, 3.665 mmol, 1 equiv) dropwise at room temperature under nitrogen atmosphere. The resulting mixture was stirred at room temperature for additional 2 h. The precipitated solids were collected by filtration and washed with DCM (5 mL×3). This resulted in 7-{[4-(benzyloxy) phenyl]methyl}-7-azaspiro [3.5]nonan-2-amine (1.3 g, 105.43% yield) as a light yellow solid. The crude product was used in the next step directly without further purification.

Step 3: To a stirred solution of 7-{[4-(benzyloxy) phenyl]methyl}-7-azaspiro [3.5]nonan-2-amine (1.3 g, 3.864 mmol, 1 equiv) and palladium (0.3 g, 2.819 mmol, 0.73 equiv) in MeOH (15 mL) at room temperature under hydrogen atmosphere. The resulting mixture was stirred at room temperature for additional 2 h under hydrogen atmosphere. The resulting mixture was filtered, the filter cake was washed with MeOH (5 mL×3). This resulted in 4-({2-amino-7-azaspiro [3.5]nonan-7-yl}methyl) phenol (1 g, 105.06% yield) as a light yellow oil. The crude product was used in the next step directly without further purification.

Step 4: To a stirred solution of 4-({2-amino-7-azaspiro [3.5]nonan-7-yl}methyl) phenol (1 g, 4.059 mmol, 1 equiv) and maleic anhydride (0.60 g, 6.088 mmol, 1.5 equiv) in MeCN (10 mL) were added TEA (1.23 g, 12.177 mmol, 3 equiv) and dropwise at room temperature under nitrogen atmosphere. The resulting mixture was stirred at 80° C. for additional overnight. The mixture was allowed to cool down to room temperature. The resulting mixture was concentrated under reduced pressure. The residue was purified by reversed-phase flash chromatography with the following conditions: column, C18 silica gel; mobile phase, MeCN in Water (0.1% FA), 10% to 50% gradient in 10 min; detector, UV 254 nm. This resulted in (2Z)-3-({7-[(4-hydroxyphenyl) methyl]-7-azaspiro [3.5]nonan-2-yl}carbamoyl)prop-2-enoic acid (590 mg, 42.20% yield) as a light yellow oil.

Step 5: To a stirred solution of (2Z)-3-({7-[(4-hydroxyphenyl) methyl]-7-azaspiro [3.5]nonan-2-yl}carbamoyl) prop-2-enoic acid (590 mg, 1.713 mmol, 1 equiv) and (2Z)-3-({7-[(4-hydroxyphenyl) methyl]-7-azaspiro [3.5]nonan-2-yl}carbamoyl) prop-2-enoic acid (590 mg, 1.713 mmol, 1 equiv) in AcOH (6 mL) at room temperature under nitrogen atmosphere. The resulting mixture was stirred at 100° C. for additional overnight. The mixture was allowed to cool down to room temperature. The resulting mixture was concentrated under reduced pressure. The residue was purified by reversed-phase flash chromatography with the following conditions: column, C18 silica gel; mobile phase, MeCN in Water (0.1% FA), 10% to 40% gradient in 10 min; detector, UV 254 nm. This resulted in 1-{7-[(4-hydroxyphenyl) methyl]-7-azaspiro [3.5]nonan-2-yl}pyrrole-2,5-dione (200 mg, 35.77% yield) as a light yellow oil.

Step 6: To a stirred solution of 1-{7-[(4-hydroxyphenyl) methyl]-7-azaspiro [3.5]nonan-2-yl}pyrrole-2,5-dione (200 mg, 0.613 mmol, 1 equiv) and 3-(fluorosulfonyl)-1,2-dimethylimidazol-1-ium triflate (301.70 mg, 0.919 mmol, 1.5 equiv) in MeCN (3 mL) were added TEA (124.01 mg, 1.226 mmol, 2 equiv) dropwise at 0° C. under nitrogen atmosphere. The resulting mixture was stirred at room temperature for additional 30 min. The resulting mixture was concentrated under reduced pressure. The residue was purified by reversed-phase flash chromatography with the following conditions: column, C18 silica gel; mobile phase, MeCN in Water (0.1% FA), 10% to 90% gradient in 10 min; detector, UV 254 nm. This resulted in 4-{[2-(2,5-dioxopyrrol-1-yl)-7-azaspiro [3.5]nonan-7-yl]methyl}phenylsulfurofluoridate (80 mg, 31.97% yield, 99.8% purity) as a white solid. LCMS:(ES, m/z): [M+H]⁺=409.1.

¹H NMR- (300 MHz, DMSO-d₆, ppm) δ 7.81-7.61 (m, 4H), 6.98 (s, 2H), 4.45 (q, J=8.8 Hz, 1H), 4.14 (s, 2H), 2.73 (s, 4H), 2.47-2.37 (m, 2H), 2.13 (s, 2H), 1.81 (s, 4H).

### Example 116. Synthesis of 4-((2-(2-(2,5-dioxo-2,5-dihydro-1H-pyrrol-1-yl)ethoxy)ethyl) carbamoyl)phenyl sulfurofluoridate (MFS-11-3)

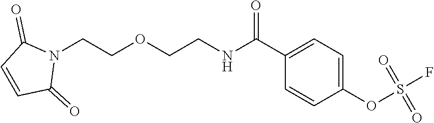 [SMILES: O=C(NCCOCCN1C(=O)C=CC1=O)c1ccc(OS(=O)(=O)F)cc1]

Step 1: To a stirred solution of tert-butyl N-[2-(2-hydroxyethoxy)ethyl]carbamate (1 g, 4.872 mmol, 1 equiv), maleimide (0.57 g, 5.846 mmol, 1.2 equiv) and PPH3 (1.92 g, 7.308 mmol, 1.5 equiv) in THF (15 mL) were added DIAD (1.48 g, 7.308 mmol, 1.5 equiv) dropwise at 0° C. under nitrogen atmosphere. The resulting mixture was stirred at room temperature for additional 2 h. The resulting mixture was diluted with 15 mL H2O and extracted with EtOAc (15 mL×3). The combined organic layers were washed with brine (15 mL), dried over anhydrous Na2SO4. The residue was purified by silica gel column chromatography, eluted with PE/EA (2:1) to afford tert-butyl N-{2-[2-(2,5-dioxopyrrol-1-yl) ethoxy]ethyl}carbamate (1 g, 72.20% yield) as a light yellow oil.

Step 2: To a stirred solution of tert-butyl N-{2-[2-(2,5-dioxopyrrol-1-yl)ethoxy]ethyl}carbamate (500 mg, 1.759 mmol, 1 equiv) and benzenesulfonic acid (417.24 mg, 2.638 mmol, 1.5 equiv) in ACN (5 mL) at room temperature under nitrogen atmosphere. The resulting mixture was stirred at room temperature for additional 1 h. The precipitated solids were collected by filtration and washed with ACN (3 mL×3). This resulted in 1-[2-(2-aminoethoxy)ethyl]pyrrole-2,5-dione (300 mg, 92.61% yield) as a white solid. The crude product was used in the next step directly without further purification.

Step 3: To a stirred solution of 1-[2-(2-aminoethoxy)ethyl]pyrrole-2,5-dione (300 mg, 1.629 mmol, 1 equiv), HATU (928.94 mg, 2.444 mmol, 1.5 equiv) and DIEA (315.76 mg, 2.444 mmol, 1.5 equiv) in DCM (5 mL) at room temperature under nitrogen atmosphere. The mixture was stirred for 15 min. 3-[(fluorosulfonyl)oxy]benzoic acid (430.31 mg, 1.955 mmol, 1.2 equiv) was added and stirred for 30 min. The resulting mixture was diluted with 5 mL H2O and extracted with DCM (5 mL×3). The combined organic layers were washed with brine (5 mL), dried over anhydrous Na2SO4. The resulting mixture was concentrated under reduced pressure. The residue was purified by reversed-phase flash chromatography with the following conditions: column, C18 silica gel; mobile phase, MeCN in Water (0.1% FA), 10% to 80% gradient in 10 min; detector, UV 254 nm. This resulted in 4-({2-[2-(2,5-dioxopyrrol-1-yl)ethoxy]ethyl}carbamoyl)phenyl sulfurofluoridate (70 mg, 11.12% yield, 96.9% purity) as a white solid. LCMS: [M+H]⁺=387.1.

¹H NMR-PH-TOPAZ-MFS-11-3-0 (300 MHz, DMSO-d₆) δ 8.61 (t, J=5.5 Hz, 1H), 8.04-7.95 (m, 2H), 7.73-7.66 (m, 2H), 6.96 (s, 2H), 3.57-3.48 (m, 6H), 3.38-3.33 (m, 2H).

### Example 117. Synthesis of 2-((2-bromoacetamido)methyl)phenyl sulfurofluoridate (BAFS-3-1)

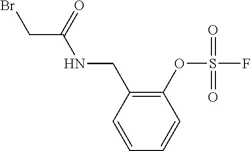 [SMILES: O=C(CBr)NCc1ccccc1OS(=O)(=O)F]

Step 1: To a stirred mixture of bromoacetyl bromide (500 mg, 2.477 mmol, 1.00 equiv) in AcOH (6 mL) and saturated aqueous solution of NaOAc (6 mL) was added 2-(aminomethyl)phenol (488.12 mg, 3.963 mmol, 1.60 equiv) in portions at 0° C. The resulting mixture was stirred at 25° C. for additional 1 h. The resulting mixture was extracted with EtOAc (3×5 mL). dried over anhydrous Na₂SO₄. After filtration, the filtrate was concentrated under reduced pressure. The residue was purified by silica gel column chromatography, eluted with PE/THF (3:1) to afford 2-bromo-N-[(2-hydroxyphenyl)methyl]acetamide (390 mg, 64.50% yield) as a colorless oil.

Step 2: To a stirred solution/mixture of 2-bromo-N-[(2-hydroxyphenyl)methyl]acetamide (250 mg, 1.024 mmol, 1 equiv) and 3-(fluorosulfonyl)-1,2-dimethylimidazol-1-ium triflate (403.44 mg, 1.229 mmol, 1.2 equiv) in ACN (8 mL) was added TEA (207.29 mg, 2.048 mmol, 2 equiv) dropwise at 0° C. under. The resulting mixture was stirred at 25° C. for additional 0.5 h. Desired product could be detected by LCMS. The resulting mixture was concentrated under vacuum. The residue was purified by reversed-phase flash chromatography with the following conditions: column, C18 silica gel; mobile phase, ACN in Water (0.1% FA), 10% to 60% gradient in 10 min; detector, UV 254 nm. This resulted in 2-[(2-bromoacetamido)methyl]phenyl sulfurofluoridate (72 mg, 21.55% yield) as a white solid. LCMS: (ES, m/z): [M+H]⁺=326.0. H NMR (400 MHz, Chloroform-d, ppm) δ 7.55-7.48 (m, 1H), 7.47-7.34 (m, 3H), 6.95 (S, 1H), 4.61 (d, J=6.1 Hz, 2H), 3.49 (s, 2H).

### Example 118. Synthesis of 3-(2-bromoacetamido)phenyl sulfurofluoridate (BAFS-3-2)

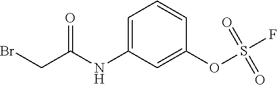 [SMILES: O=C(CBr)Nc1cccc(OS(=O)(=O)F)c1]

Step 1: To a stirred solution of m-aminophenol (1 g, 9.16 mmol, 1.00 equiv) and bromoacetyl bromide (2.96 g, 14.66 mmol, 1.60 equiv) in AcOH (10 mL) and saturated aqueous solution of AcONa (10 mL) was added bromoacetyl bromide (2.96 g, 14.66 mmol, 1.60 equiv) dropwise at 0° C. under nitrogen atmosphere. The resulting mixture was stirred at room temperature for additional 1 h. The resulting mixture was diluted with 20 mL H₂O and extracted with EtOAc (3×20 mL). The combined organic layer was washed with brine (20 mL), dried over anhydrous Na₂SO₄. After filtration, the filtrate was concentrated under reduced pressure. The residue was purified by silica gel column chromatography, eluted with PE/THF (3/1) to afford 2-bromo-N-(3-hydroxyphenyl) acetamide (1 g, 47.43% yield) as a light brown oil.

Step 2: To a stirred solution of 2-bromo-N-(3-hydroxyphenyl) acetamide (1 g, 4.35 mmol, 1.00 equiv) and 3-(fluorosulfonyl)-1,2-dimethylimidazol-1-ium triflate (2.85 g, 8.69 mmol, 2.00 equiv) in MeCN (15 mL) were added TEA (1.32 g, 13.04 mmol, 3.00 equiv) dropwise at 0° C. The resulting mixture was stirred at room temperature for additional 1 h. The resulting mixture was concentrated under reduced pressure. The residue was purified by reversed-phase flash chromatography with the following conditions: column, C18 silica gel; mobile phase, MeCN in Water (0.1% FA), 10% to 95% gradient in 10 min; detector, UV 254 nm. This resulted in 3-(2-bromoacetamido) phenyl sulfurofluoridate (350 mg, 25.80% yield, 99.7% purity) as a white solid. LCMS: (ES, m/z): [M−H]⁻=309.8. ¹HNMR (300 MHz, DMSO-d₆, ppm) δ 10.80 (s, 1H), 7.98-7.91 (m, 1H), 7.64-7.51 (m, 2H), 7.40-7.28 (m, 1H), 4.08 (s, 2H).

### Example 119. Synthesis of 2-((2-bromoacetamido)methyl)-6-fluorophenyl sulfurofluoridate (BAFS-3-3)

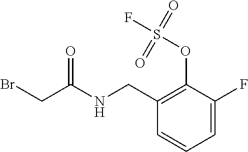 [SMILES: O=C(CBr)NCc1cccc(F)c1OS(=O)(=O)F]

Step 1: Into a 40 mL sealed tube were added 3-fluoro-2-hydroxybenzonitrile (1 g, 7.29 mmol, 1 equiv) and THF (10 mL) at room temperature. To the above mixture was added Lithium aluminum hydriden (2.0 M in TIF) (0.55 g, 14.58 mmol, 2 equiv) dropwise over 3 min at 0° C. The resulting mixture was stirred at 0° C. for additional 1 h. The reaction was quenched by the addition of water (5 mL) at 0° C. The mixture/residue was neutralized to pH 4 with conc. HCl. The residue was purified by reversed-phase flash chromatography with the following conditions: column, C18 silica gel; mobile phase, MeCN in Water (0.1% FA), 10% to 50% gradient in 10 min; detector, UV 254 nm. This resulted in 2-(aminomethyl)-6-fluorophenol (600 mg, 58.29% yield, 95% purity) as a yellow oil.

Step 2: Into a mL sealed tube were added 2-(aminomethyl)-6-fluorophenol (600 mg, 4.25 mmol, 1 equiv) and Sodium acetate trihydrate (5 mL) at room temperature. To the above mixture was added bromoacetyl bromide (1029.64 mg, 5.10 mmol, 1.2 equiv) dropwise over 3 min at 0° C. The resulting mixture was stirred at room temperature for additional 1 h. The resulting mixture was concentrated under reduced pressure. The residue was purified by reversed-phase flash chromatography with the following conditions: column, C18 silica gel; mobile phase, MeCN in Water (0.1% FA), 10% to 50% gradient in 10 min; detector, UV 254 nm. This resulted in 2-bromo-N-[(3-fluoro-2-hydroxyphenyl)methyl]acetamide (300 mg, 26.93% yield, 96% purity) as a yellow solid.

Step 3: Into a 40 mL sealed tube were added 2-bromo-N-[(3-fluoro-2-hydroxyphenyl)methyl]acetamide (300 mg, 1.14 mmol, 1 equiv), MeCN (5 mL) and 1-(fluorosulfonyl)-2,3-dimethyl-1H-imidazol-3-ium trifluoromethanesulfonate (488.47 mg, 1.48 mmol, 1.3 equiv) at room temperature. To the above mixture was added Et3N (347.51 mg, 3.43 mmol, 3 equiv) dropwise over 3 min at 0° C. The resulting mixture was stirred at 0° C. for additional 1 h. The residue was purified by reversed-phase flash chromatography with the following conditions: column, C18 silica gel; mobile phase, MeCN in Water (0.1% FA), 10% to 50% gradient in 10 min; detector, UV 254 nm. This resulted in 2-[(2-bromoacetamido)methyl]-6-fluorophenyl sulfurofluoridate (153.2 mg, 38.89% yield, 98.9% purity) as a white solid. LCMS:(ES, m/z): [M+ACN]⁺=369.9.

¹H NMR (300 MHz, Chloroform-d) δ 7.38 (td, J=8.0, 4.9 Hz, 1H), 7.32-7.27 (m, 1H), 7.25-7.19 (m, 1H), 6.94 (s, 1H), 4.62 (d, J=6.2 Hz, 2H), 3.93 (s, 2H)

### Example 120. Synthesis of 3-(2-bromoacetamido)-2-fluorophenyl sulfurofluoridate (BAFS-3-4)

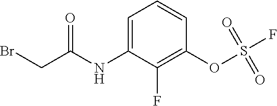 [SMILES: O=C(CBr)Nc1cccc(OS(=O)(=O)F)c1F]

Step 1: To a stirred mixture of 2-fluoro-3-nitrophenol (500 mg, 3.183 mmol, 1 equiv) and Pd/C (67.74 mg, 0.637 mmol, 0.2 equiv) in MeOH (10 mL) under hydrogen atmosphere. The resulting mixture was stirred at room temperature for 1 h. The resulting mixture was filtered, the filter cake was washed with MeOH (3×5 mL). The filtrate was concentrated under reduced pressure to afford 3-amino-2-fluorophenol (300 mg, 74.15% yield) as a yellow solid. The crude resulting mixture was used in the next step directly without further purification.

Step 2: To a stirred solution of 3-amino-2-fluorophenol (500 mg, 3.933 mmol, 1 equiv) in AcOH (5 mL) and saturated aqueous solution of AcONa (5 mL) were added bromoacetyl bromide (1.19 g, 3.933 mmol, 1.5 equiv) in portions at 0° C. The resulting mixture was stirred at room temperature for 1 h. The resulting mixture was diluted with 10 mL H₂O and extracted with EtOAc (3×10 mL). The combined organic layer was washed with brine (10 mL), dried over anhydrous Na₂SO₄. After filtration, the filtrate was concentrated under reduced pressure. The residue was purified by silica gel column chromatography, eluted with PE/THF (3:1) to afford 2-bromo-N-(2-fluoro-3-hydroxyphenyl)acetamide (300 mg, 34.16% yield) as a brown solid.

Step 3: To a stirred solution of A solution of 2-bromo-N-(2-fluoro-3-hydroxyphenyl)acetamide (250 mg, 1.008 mmol, 1 equiv) and 3-(fluorosulfonyl)-1,2-dimethylimidazol-1-ium triflate (396.99 mg, 1.210 mmol, 1.2 equiv) in ACN (5 mL) was added TEA (152.98 mg, 1.512 mmol, 1.5 equiv) dropwise at 0° C. The resulting mixture was stirred at room temperature for 1 h. The resulting mixture was concentrated under reduced pressure. The residue was purified by reversed-phase flash chromatography with the following conditions: column, C18 silica gel; mobile phase, MeCN in Water (0.1% FA), 10% to 95% gradient in 10 min; detector, UV 254 nm. This resulted in 3-(2-bromoacetamido)-2-fluorophenyl sulfurofluoridate (100 mg, 30.06% yield, 99.5% purity) as a white solid.

¹H NMR (300 MHz, Chloroform-d, ppm) δ 8.45 (s, 1H), 8.37 (td, J=7.3, 6.7, 2.0 Hz, 1H), 7.26-7.15 (m, 2H), 4.07 (s, 2H).

### Example 121. Synthesis of 4-(2-bromoacetamido)phenyl sulfurofluoridate (BAFS-4-1)

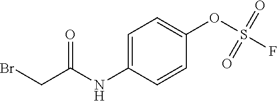 [SMILES: O=C(CBr)Nc1ccc(OS(=O)(=O)F)cc1]

Step 1: To a stirred solution of aminophenol (500 mg, 4.582 mmol, 1 equiv) in AcOH (5 mL) and saturated aqueous solution of AcONa (5 mL) was added bromoacetyl bromide (1479.69 mg, 7.331 mmol, 1.6 equiv) dropwise at 0° C. under nitrogen atmosphere. The resulting mixture was stirred at room temperature for additional 1 h. The resulting mixture was diluted with 10 mL H₂O and extracted with EtOAc (10 mL×3). The combined organic layer was washed with brine (10 mL), dried over anhydrous Na₂SO₄. After filtration, the filtrate was concentrated under reduced pressure. The residue was purified by silica gel column chromatography, eluted with PE/THF (3:1) to afford 2-bromo-N-(4-hydroxyphenyl)acetamide (300 mg, 28.46% yield) as a light yellow oil.

Step 2: To a stirred solution of 2-bromo-N-(4-hydroxyphenyl) acetamide (300 mg, 1.304 mmol, 1 equiv) and 3-(fluorosulfonyl)-1,2-dimethylimidazol-1-ium triflate (642.06 mg, 1.956 mmol, 1.5 equiv) in MeCN (5 mL) were added TEA (263.91 mg, 2.608 mmol, 2 equiv) dropwise at 0° C. under nitrogen atmosphere. The resulting mixture was stirred at room temperature for additional 30 min. The resulting mixture was concentrated under reduced pressure. The residue was purified by reversed-phase flash chromatography with the following conditions: column, C18 silica gel; mobile phase, MeCN in Water (0.1% FA), 10% to 90% gradient in 10 min; detector, UV 254 nm. This resulted in 4-(2-bromoacetamido) phenyl sulfurofluoridate (100 mg, 24.57% yield, 99.5% purity) as a white solid. LCMS: (ES, m/z): [M+H]⁺=311.8.

¹H NMR (300 MHz, Chloroform-d, ppm,) δ 8.21 (s, 1H), 7.72-7.63 (m, 2H), 7.42-7.30 (m, 2H), 4.05 (s, 2H).

### Example 122. Synthesis of 4-(2-bromoacetamido)-2-fluorophenyl sulfurofluoridate (BAFS-4-2)

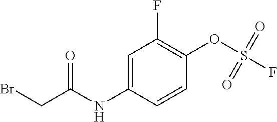 [SMILES: O=C(CBr)Nc1ccc(OS(=O)(=O)F)c(F)c1]

Step 1: To a stirred solution of 4-amino-2-fluorophenol (1 g, 7.867 mmol, 1 equiv) in AcOH (10 mL) and saturated aqueous solution of AcONa (10 mL) were added 4-(aminomethyl)phenol (2.54 g, 12.587 mmol, 1.6 equiv) in portions at 0° C. The resulting mixture was stirred at room temperature for 1 h. The resulting mixture was diluted with 20 mL H₂O and extracted with EtOAc (3×20 mL). The combined organic layer was washed with brine (20 mL), dried over anhydrous Na₂SO₄. After filtration, the filtrate was concentrated under reduced pressure. The residue was purified by silica gel column chromatography, eluted with PE/THF (3:1) to afford 2-bromo-N-(3-fluoro-4-hydroxyphenyl)acetamide (1.2 g, 61.50% yield) as a light brown oil.

Step 2: To a stirred solution of 2-bromo-N-(3-fluoro-4-hydroxyphenyl)acetamide (250 mg, 1.008 mmol, 1 equiv) and 3-(fluorosulfonyl)-1,2-dimethylimidazol-1-ium triflate (396.99 mg, 1.210 mmol, 1.2 equiv) in ACN (5 mL) was added TEA (152.98 mg, 1.512 mmol, 1.5 equiv) dropwise at 0° C. The resulting mixture was stirred at room temperature for 1 h. The resulting mixture was concentrated under reduced pressure. The residue was purified by reversed-phase flash chromatography with the following conditions: column, C18 silica gel; mobile phase, MeCN in Water (0.1% FA), 10% to 50% gradient in 10 min; detector, UV 254 nm. This resulted in 4-(2-bromoacetamido)-2-fluorophenyl sulfurofluoridate (100 mg, 30.06% yield, 99.2% purity) as a yellow solid. LCMS: [M+H]⁺=330.1.

¹H NMR (300 MHz, Chloroform-d, ppm) δ 8.22 (s, 1H), 7.81 (dd, J=11.5, 2.5 Hz, 1H), 7.40 (ddd, J=9.0, 7.8, 1.1 Hz, 1H), 7.28-7.24 (m, 1H), 4.04 (s, 2H).

### Example 123. Synthesis of 3-((2-bromoacetamido)methyl)phenyl sulfurofluoridate (BAFS-4-3)

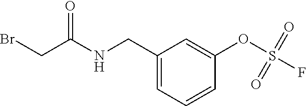 [SMILES: O=C(CBr)NCc1cccc(OS(=O)(=O)F)c1]

Step 1: Into a mL sealed tube were added 3-(aminomethyl)phenol (1 g, 8.12 mmol, 1 equiv), HOAc (5 mL) and Sodium acetate trihydrate (5 mL) at room temperature. To the above mixture was added bromoacetyl bromide (1.97 g, 9.74 mmol, 1.2 equiv) dropwise over 3 min at 0° C. The resulting mixture was stirred at room temperature for additional 1 h. The resulting mixture was concentrated under reduced pressure. The residue was purified by reversed-phase flash chromatography with the following conditions: column, C18 silica gel; mobile phase, MeCN in Water (0.1% FA), 10% to 50% gradient in 10 min; detector, UV 254 nm. This resulted in 2-bromo-N-[(3-hydroxyphenyl)methyl]acetamide (400 mg, 20.18% yield, 95% purity) as a yellow oil.

Step 2: Into a 40 mL sealed tube were added 2-bromo-N-[(3-hydroxyphenyl)methyl]acetamide (400 mg, 1.63 mmol, 1 equiv), 1-(fluorosulfonyl)-2,3-dimethyl-1H-imidazol-3-ium trifluoromethanesulfonate (699.30 mg, 2.13 mmol, 1.3 equiv) and MeCN (10 mL) at room temperature. To the above mixture was added Et3N (497.49 mg, 4.91 mmol, 3 equiv) dropwise over 3 min at 0° C. The resulting mixture was stirred at room temperature for additional 1 h. The residue was purified by reversed-phase flash chromatography with the following conditions: column, C18 silica gel; mobile phase, MeCN in Water (0.1% FA), 10% to 50% gradient in 10 min; detector, UV 254 nm. This resulted in 3-[(2-bromoacetamido)methyl]phenyl sulfurofluoridate (249.7 mg, 46.72% yield, 99.9% purity) as a white solid. LCMS:(ES, m/z): [M+ACN]⁺=369.9.

¹H NMR (300 MHz, Chloroform-d) δ 7.53-7.42 (m, 1H), 7.36 (d, J=7.7 Hz, 1H), 7.29 (d, J=7.5 Hz, 2H), 6.88 (m, 1H), 4.54 (d, J=6.1 Hz, 2H), 3.96 (s, 2H).

### Example 124. Synthesis of 2-(2-(2-bromoacetamido)ethyl)phenyl sulfurofluoridate (BAFS-4-4)

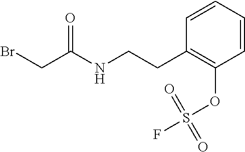 [SMILES: O=C(CBr)NCCc1ccccc1OS(=O)(=O)F]

Step 1: To a stirred solution of 2-hydroxyphenethylamine (500 mg, 3.645 mmol, 1 equiv) in AcOH (5 mL) and saturated aqueous solution of AcONa (5 mL) were added bromoacetyl bromide (1.10 g, 5.468 mmol, 1.5 equiv) in portions at 0° C. The resulting mixture was stirred at room temperature for 1 h. The resulting mixture was diluted with 10 mL H₂O and extracted with EtOAc (3×10 mL). The combined organic layer was washed with brine (10 mL), dried over anhydrous Na₂SO₄. After filtration, the filtrate was concentrated under reduced pressure. The residue was purified by silica gel column chromatography, eluted with PE/THF (3:1) to afford 22-bromo-N-[2-(2-hydroxyphenyl)ethyl]acetamide (400 mg, 42.52% yield) as a yellow oil.

Step 2: To a stirred solution of A solution of 2-bromo-N-[2-(2-hydroxyphenyl)ethyl]acetamide (200 mg, 0.775 mmol, 1 equiv) and 3-(fluorosulfonyl)-1,2-dimethylimidazol-1-ium triflate (305.21 mg, 0.930 mmol, 1.2 equiv) in ACN (5 mL) was added TEA (117.61 mg, 1.163 mmol, 1.5 equiv) dropwise at 0° C. The resulting mixture was stirred at room temperature for 1 h. The resulting mixture was concentrated under reduced pressure. The residue was purified by reversed-phase flash chromatography with the following conditions: column, C18 silica gel; mobile phase, MeCN in Water (0.1% FA), 10% to 95% gradient in 10 min; detector, UV 254 nm. This resulted in 2-[2-(2-bromoacetamido)ethyl]phenyl sulfurofluoridate (100 mg, 37.94% yield, 98.0% purity) as a white solid. LCMS: (ES, m/z): [M+H]⁺=339.9.

¹H NMR (400 MHz, Chloroform-d, ppm) δ 7.38 (m, 4H), 6.66 (s, 1H), 3.87 (s, 2H), 3.60 (q, J=6.8 Hz, 2H), 2.99 (t, J=7.3 Hz, 2H).

### Example 125. Synthesis of 4-((2-bromoacetamido)methyl)phenyl sulfurofluoridate (BAFS-5-1)

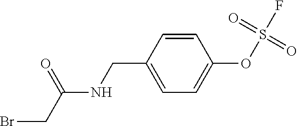 [SMILES: O=C(CBr)NCc1ccc(OS(=O)(=O)F)cc1]

Step 1: To a stirred solution of bromoacetyl bromide (1 g, 4.954 mmol, 1 equiv) in AcOH (10 mL) and saturated aqueous solution of AcONa (10 mL) were added 4-(aminomethyl)phenol (0.98 g, 7.926 mmol, 1.6 equiv) in portions at 0° C. The final reaction mixture was irradiated with microwave radiation at room temperature for 1 h. The resulting mixture was diluted with 20 mL H₂O and extracted with EtOAc (3×20 mL). The combined organic layer was washed with brine (20 mL), dried over anhydrous Na₂SO₄. After filtration, the filtrate was concentrated under reduced pressure. The residue was purified by silica gel column chromatography, eluted with PE/THF (3:1) to afford 2-bromo-N-[(4-hydroxyphenyl)methyl]acetamide (1 g, 82.69% yield) as a light brown oil.

Step 2: To a stirred solution of 2-bromo-N-[(4-hydroxyphenyl)methyl]acetamide (1 g, 4.097 mmol, 1 equiv) and 3-(fluorosulfonyl)-1,2-dimethylimidazol-1-ium triflate (2.69 g, 8.194 mmol, 2 equiv) in ACN (15 mL) was added TEA (1.24 g, 12.291 mmol, 3 equiv) dropwise at 0° C. The final reaction mixture was irradiated with microwave radiation at room temperature for 1 h. The resulting mixture was concentrated under reduced pressure. The residue was purified by reversed-phase flash chromatography with the following conditions: column, C18 silica gel; mobile phase, MeCN in Water (0.1% FA), 10% to 50% gradient in 10 min; detector, UV 254 nm. This resulted in 4-[(2-bromoacetamido)methyl]phenyl sulfurofluoridate (350 mg, 26.19% yield, 95% purity) as a white solid.

¹H NMR (300 MHz, Chloroform-d) δ 7.41 (d, J=8.7 Hz, 2H), 7.33 (dd, J=8.9, 0.9 Hz, 2H), 6.91 (s, 1H), 4.52 (d, J=6.0 Hz, 2H), 3.95 (s, 2H).

### Example 126. Synthesis of 2-(2-bromoacetyl)phenyl sulfurofluoridate (BFS-2-1)

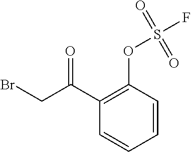 [SMILES: O=C(CBr)c1ccccc1OS(=O)(=O)F]

Step 1: To a stirred mixture of o-acetylphenol (500 mg, 3.672 mmol, 1.0 equiv) and 3-(fluorosulfonyl)-1,2-dimethylimidazol-1-ium triflate (1446.57 mg, 4.406 mmol, 1.2 equiv) in ACN (12 mL) was added TEA (743.25 mg, 7.344 mmol, 2.0 equiv) dropwises at 0° C. The resulting mixture was stirred at 25° C. for additional 30 min. The reaction was monitored by LCMS. The resulting mixture was concentrated under vacuum. The residue was purified by reversed-phase flash chromatography with the following conditions: column, C18 silica gel; mobile phase, MeCN in Water (0.1% FA), 10% to 60% gradient in 10 min; detector, UV 254 nm. This resulted in 4-acetylphenyl sulfurofluoridate (400 mg, 49.92% yield) as a white solid.

Step 2: To a stirred mixture of 2-acetylphenyl sulfurofluoridate (250 mg, 1.146 mmol, 1 equiv) and NBS (305.89 mg, 1.719 mmol, 1.5 equiv) in ACN were added TsOH (19.73 mg, 0.115 mmol, 0.1 equiv). The resulting mixture was stirred at 80° C. for additional 2 h. Desired product could be detected by LCMS. The resulting mixture was concentrated under vacuum. The residue was purified by reversed-phase flash chromatography with the following conditions: column, C18 silica gel; mobile phase, ACN in Water (0.1% FA), 10% to 60% gradient in 10 min; detector, UV 254 nm. This resulted in 2-(2-bromoacetyl)phenyl sulfurofluoridate (60 mg, 17.63% yield) as a light yellow oil.

¹H NMR (400 MHz, Chloroform-d, ppm) δ 7.89 (dd, J=7.8, 1.7 Hz, 1H), 7.70 (td, J=7.9, 1.7 Hz, 1H), 7.56 (td, J=7.6, 1.2 Hz, 1H), 7.49 (dt, J=8.2, 1.4 Hz, 1H), 4.43 (s, 2H).

### Example 127. Synthesis of 2-(2-bromoacetyl)-6-fluorophenyl sulfurofluoridate (BAFS-2-2)

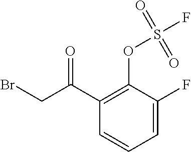 [SMILES: O=C(CBr)c1cccc(F)c1OS(=O)(=O)F]

Step 1: Into a 40 mL sealed tube were added 1-(3-fluoro-2-hydroxyphenyl)ethanone (500 mg, 3.244 mmol, 1 equiv), MeCN (10 mL) and 3-(fluorosulfonyl)-1,2-dimethylimidazol-1-ium triflate (1384.21 mg, 4.21 mmol, 1.3 equiv) at room temperature. To the above mixture was added Et3N (984.75 mg, 9.73 mmol, 3 equiv) dropwise over 3 min at 0° C. The resulting mixture was stirred at 0° C. for additional 1 h. The residue was purified by reversed-phase flash chromatography with the following conditions: column, C18 silica gel; mobile phase, MeCN in Water (0.1% FA), 10% to 50% gradient in 10 min; detector, UV 254 nm. This resulted in 2-acetyl-6-fluorophenyl sulfurofluoridate (400 mg, 52.21% yield, 95% purity) as a yellow oil.

Step 2: Into a 40 mL sealed tube were added 2-acetyl-6-fluorophenyl sulfurofluoridate (400 mg, 1.69 mmol, 1 equiv), MeCN (5 mL), TSOH (29.16 mg, 0.17 mmol, 0.1 equiv) and NBS (452.14 mg, 2.54 mmol, 1.5 equiv) at room temperature. The resulting mixture was stirred at 80° C. for 4 h under nitrogen atmosphere. The mixture was allowed to cool down to room temperature. The residue was purified by reversed-phase flash chromatography with the following conditions: column, C18 silica gel; mobile phase, MeCN in Water (0.1% FA), 10% to 50% gradient in 10 min; detector, UV 254 nm. This resulted in 2-(2-bromoacetyl)-6-fluorophenyl sulfurofluoridate (185.0 mg, 34.67% yield, 97.0% purity) as a white solid.

¹H NMR (300 MHz, Chloroform-d) δ 7.69-7.59 (m, 1H), 7.59-7.45 (m, 2H), 4.40 (s, 2H).

### Example 128. Synthesis of 3-(2-bromoacetyl)phenyl sulfurofluoridate (BFS-3-1)

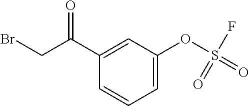 [SMILES: O=C(CBr)c1cccc(OS(=O)(=O)F)c1]

Step 1: To a stirred solution of 3-hydroxyacetophenone (500 mg, 3.67 mmol, 1.00 equiv) and 3-(fluorosulfonyl)-1,2-dimethylimidazol-1-ium triflate (2.41 g, 7.34 mmol, 2.00 equiv) in ACN (10 mL) were added TEA (1.11 g, 11.02 mmol, 3.00 equiv) dropwise at 0° C. under nitrogen atmosphere. The resulting mixture was stirred at room temperature for additional 1 h. The resulting mixture was concentrated under reduced pressure. The residue was purified by silica gel column chromatography, eluted with PE/THF (3:1) to afford 3-acetylphenyl sulfurofluoridate (400 mg, 49.92% yield) as a light yellow oil.

Step 2: To a stirred solution of 3-acetylphenyl sulfurofluoridate (400 mg, 1.83 mmol, 1.00 equiv) and NBS (326.28 mg, 1.83 mmol, 1.00 equiv) in ACN (5 mL) was added TsOH (31.57 mg, 0.18 mmol, 0.10 equiv) in portions at 0° C. under nitrogen atmosphere. The resulting mixture was stirred at 80° C. for additional 3 h. The mixture was allowed to cool down to room temperature. The resulting mixture was concentrated under reduced pressure. The residue was purified by reversed-phase flash chromatography with the following conditions: column, C18 silica gel; mobile phase, MeCN in Water (0.1% FA), 10% to 95% gradient in 10 min; detector, UV 254 nm. This resulted in 3-(2-bromoacetyl)phenyl sulfurofluoridate (100 mg, 18.36% yiel, 99.2% purity) as a white solid ¹H NMR (300 MHz, DMSO-d₆, ppm) δ 8.20-8.13 (m, 2H), 7.95 (ddt, J=8.3, 2.3, 1.0 Hz, 1H), 7.81 (dd, J=8.4, 7.6 Hz, 1H), 5.00 (s, 2H).

### Example 129. Synthesis of 3-(2-bromoacetyl)-2-fluorophenyl sulfurofluoridate (BFS-3-3)

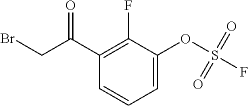 [SMILES: O=C(CBr)c1cccc(OS(=O)(=O)F)c1F]

Step 1: Into a 40 mL sealed tube were added 1-(2-fluoro-3-hydroxyphenyl)ethanone (500 mg, 3.24 mmol, 1.00 equiv), MeCN (10 mL) and 3-(fluorosulfonyl)-1,2-dimethylimidazol-1-ium triflate (1384.21 mg, 4.21 mmol, 1.3 equiv) at room temperature. To the above mixture was added Et3N (984.75 mg, 9.73 mmol, 3 equiv) dropwise over 3 min at 0° C. The resulting mixture was stirred at 0° C. for additional 1 h. The residue was purified by reversed-phase flash chromatography with the following conditions: column, C18 silica gel; mobile phase, MeCN in Water (0.1% FA), 10% to 50% gradient in 10 min; detector, UV 254 nm. This resulted in 3-acetyl-2-fluorophenyl sulfonate (400 mg, 56.51% yield, 99% purity) as a yellow oil.

Step 2: Into a 40 mL sealed tube were added 3-acetyl-2-fluorophenyl sulfurofluoridate (400 mg, 1.69 mmol, 1 equiv), MeCN (5 mL), TSOH (29.16 mg, 0.16 mmol, 0.1 equiv), MeCN (5 mL) and NBS (452.14 mg, 2.54 mmol, 1.5 equiv) at room temperature. The resulting mixture was stirred at 80° C. for 4 h under nitrogen atmosphere. The mixture was allowed to cool down to room temperature. The residue was purified by reversed-phase flash chromatography with the following conditions: column, C18 silica gel; mobile phase, MeCN in Water (0.1% FA), 10% to 50% gradient in 10 min; detector, UV 254 nm. This resulted in 3-(2-bromoacetyl)-2-fluorophenyl sulfurofluoridate (183.3 mg, 34.35% yield, 99.4% purity) as a white solid.

¹H NMR (300 MHz, Chloroform-d) δ 8.00 (ddd, J=8.0, 6.2, 1.8 Hz, 1H), 7.68 (ddt, J=8.3, 6.9, 1.4 Hz, 1H), 7.40 (td, J=8.1, 1.4 Hz, 1H), 4.50 (d, J=2.4 Hz, 2H).

### Example 130. Synthesis of 4-(2-bromoacetyl)phenyl sulfurofluoridate (BFS-4-1)

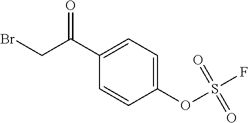 [SMILES: O=C(CBr)c1ccc(OS(=O)(=O)F)cc1]

Step 1: To a stirred solution of hydroxyacetophenone (500 mg, 3.672 mmol, 1.00 equiv) and 3-(fluorosulfonyl)-1,2-dimethylimidazol-1-ium triflate (1446.57 mg, 4.407 mmol, 1.20 equiv) in ACN (12 mL) was added TEA (743.25 mg, 7.344 mmol, 2 equiv) dropwises at 0° C. The resulting mixture was stirred at 25° C. for additional 30 min. The reaction was monitored by LCMS. The resulting mixture was concentrated under vacuum. The residue was purified by reversed-phase flash chromatography with the following conditions: column, C18 silica gel; mobile phase, MeCN in Water (0.1% FA), 10% to 60% gradient in 10 min; detector, UV 254 nm. This resulted in 4-acetylphenyl sulfurofluoridate (400 mg, 49.92% yield) as a white solid.

Step 2: To a stirred mixture of 4-acetylphenyl sulfurofluoridate (300 mg, 1.375 mmol, 1 equiv) and NBS (367.06 mg, 2.063 mmol, 1.5 equiv) in MeCN were added TsOH (23.68 mg, 0.138 mmol, 0.1 equiv). The resulting mixture was stirred at 80° C. for additional 2 h. Desired product could be detected by LCMS. The resulting mixture was concentrated under vacuum. The residue was purified by reversed-phase flash chromatography with the following conditions: column, C18 silica gel; mobile phase, ACN in Water (0.1% FA), 10% to 60% gradient in 10 min; detector, UV 254 nm. This resulted in 4-(2-bromoacetyl)phenyl sulfurofluoridate (120 mg, 29.38% yield) as a white solid.

¹H NMR (300 MHz, Chloroform-d, ppm) δ 8.17-8.10 (m, 2H), 7.53-7.46 (m, 2H), 4.42 (s, 2H).

### Example 131. Synthesis of 4-(2-bromoacetyl)-2-fluorophenyl sulfurofluoridate (BFS-4-2)

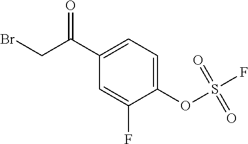 [SMILES: O=C(CBr)c1ccc(OS(=O)(=O)F)c(F)c1]

Step 1: Into a 40 mL sealed tube were added 1-(3-fluoro-4-hydroxyphenyl)ethanone (500 mg, 3.24 mmol, 1 equiv), MeCN (10 mL) and 3-(fluorosulfonyl)-1,2-dimethylimidazol-1-ium triflate (1384.21 mg, 4.21 mmol, 1.3 equiv) at room temperature. To the above mixture was added Et3N (984.75 mg, 9.732 mmol, 3 equiv) dropwise over 3 min at 0° C. The resulting mixture was stirred at 0° C. for additional 1 h. The residue was purified by reversed-phase flash chromatography with the following conditions: column, C18 silica gel; mobile phase, MeCN in Water (0.1% FA), 10% to 50% gradient in 10 min; detector, UV 254 nm. This resulted in 4-acetyl-2-fluorophenyl sulfurofluoridate (450 mg, 58.73% yield, 97% purity) as a yellow oil.

Step 2: Into a 40 mL sealed tube were added 4-acetyl-2-fluorophenyl sulfurofluoridate (450 mg, 1.90 mmol, 1 equiv), MeCN (5 mL), TSOH (32.81 mg, 0.19 mmol, 0.1 equiv) and NBS (508.66 mg, 2.85 mmol, 1.5 equiv) at room temperature. The resulting mixture was stirred at 80° C. for 3 h under nitrogen atmosphere. The mixture was allowed to cool down to room temperature. The residue was purified by reversed-phase flash chromatography with the following conditions: column, C18 silica gel; mobile phase, MeCN in Water (0.1% FA), 10% to 50% gradient in 10 min; detector, UV 254 nm. This resulted in 4-(2-bromoacetyl)-2-fluorophenyl sulfurofluoridate (209.1 mg, 34.83% yield, 99.1% purity) as a white solid.

¹H NMR (300 MHz, Chloroform-d) δ 7.98-7.84 (m, 2H), 7.58 (ddd, J=7.7, 6.7, 1.1 Hz, 1H), 4.39 (s, 2H).

### Example 132. Synthesis of 3-(2-chloroacetamido)phenyl sulfurofluoridate (CAFS-3-2)

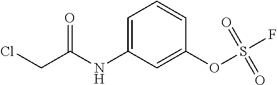 [SMILES: O=C(CCl)Nc1cccc(OS(=O)(=O)F)c1]

Step 1: To a stirred solution of m-aminophenol (500 mg, 4.58 mmol, 1.00 equiv) in DCM (5 mL) were added chloroacetyl chloride (465.72 mg, 4.12 mmol, 0.90 equiv) dropwise at 0° C. under nitrogen atmosphere. The resulting mixture was stirred at room temperature for additional 2 h. The resulting mixture was diluted with 10 mL H₂O and extracted with DCM (3×5 mL). The combined organic layer was washed with brine (5 mL), dried over anhydrous Na₂SO₄. After filtration, the filtrate was concentrated under reduced pressure. The residue was purified by silica gel column chromatography, eluted with CH₂Cl₂/THF (3:1) to afford 2-chloro-N-(3-hydroxyphenyl) acetamide (400 mg, 47.04% yield) as a light yellow oil.

Step 2: To a stirred solution of 2-chloro-N-(3-hydroxyphenyl) acetamide (400 mg, 2.16 mmol, 1.00 equiv) and 3-(fluorosulfonyl)-1,2-dimethylimidazol-1-ium triflate (1.41 g, 4.31 mmol, 2.00 equiv) in MeCN (10 mL) were added TEA (436.15 mg, 4.31 mmol, 2.00 equiv) dropwise 0° C. under nitrogen atmosphere. The resulting mixture was stirred at 0° C. for additional 30 min. The resulting mixture was concentrated under reduced pressure. The residue was purified by reversed-phase flash chromatography with the following conditions: column, C18 silica gel; mobile phase, MeCN in Water (0.1% FA), 10% to 95% gradient in 10 min; detector, UV 254 nm. This resulted in 3-(2-chloroacetamido) phenyl sulfurofluoridate (200 mg, 34.67% yield) as a white solid. LCMS: (ES, m/z): [M−H]⁻=265.8.

¹H NMR (300 MHz, DMSO-d₆, ppm) δ 10.71 (s, 1H), 7.94 (s, 1H), 7.64-7.52 (m, 2H), 7.36-7.29 (m, 1H), 4.30 (d, J=1.4 Hz, 2H).

### Example 133. Synthesis of 5-(2-bromoacetyl)-2-fluorophenyl sulfurofluoridate (BFS-3-2)

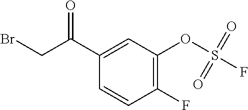 [SMILES: O=C(CBr)c1ccc(F)c(OS(=O)(=O)F)c1]

Step 1: To a stirred solution of 1-(4-fluoro-3-hydroxyphenyl)ethanone (500 mg, 3.244 mmol, 1 equiv) and 3-(fluorosulfonyl)-1,2-dimethylimidazol-1-ium triflate (2129.56 mg, 6.488 mmol, 2 equiv) in ACN (10 mL) was added TEA (1.35 mL, 9.732 mmol, 3 equiv) dropwise at 0° C. The resulting mixture was stirred at room temperature for 1 h. The resulting mixture was concentrated under reduced pressure. The residue was purified by silica gel column chromatography, eluted with PE/THF (3:1) to afford 5-acetyl-2-fluorophenyl sulfurofluoridate (400 mg, 52.21% yield) as a light yellow oil.

Step 2: To a stirred solution of 5-acetyl-2-fluorophenyl sulfurofluoridate (400 mg, 1.694 mmol, 1 equiv) and NBS (301.43 mg, 1.694 mmol, 1 equiv) in ACN (5 mL) was added TsOH (29.16 mg, 0.169 mmol, 0.1 equiv) dropwise at 0° C. The resulting mixture was stirred at 80° C. for 3 h. The mixture was allowed to cool down to room temperature. The resulting mixture was concentrated under reduced pressure. The residue was purified by reversed-phase flash chromatography with the following conditions: column, C18 silica gel; mobile phase, MeCN in Water (0.1% FA), 10% to 50% gradient in 10 min; detector, UV 254 nm. This resulted in 5-(2-bromoacetyl)-2-fluorophenyl sulfurofluoridate (100 mg, 18.74% yield, 99.4% purity) as a white solid.

¹H NMR (300 MHz, Chloroform-d) δ 8.11-8.05 (m, 2H), 7.43 (t, J=8.9 Hz, 1H), 4.38 (s, 2H).

### Example 134. Synthesis of 2-(acrylamidomethyl)phenyl sulfurofluoridate (AFS-3-2)

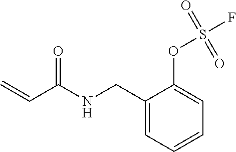 [SMILES: C=CC(=O)NCc1ccccc1OS(=O)(=O)F]

Step 1: To a stirred solution of 2-(aminomethyl) phenol (550 mg, 4.466 mmol, 1 equiv) and acryloyl chloride (404.21 mg, 4.466 mmol, 1 equiv) in DCM (5 mL) was added TEA (451.92 mg, 4.466 mmol, 1 equiv) dropwise at 0° C. under nitrogen atmosphere. The resulting mixture was stirred at room temperature for 2 h. The resulting mixture was diluted with water (10 mL). The resulting mixture was extracted with DCM (3×5 mL). The combined organic layers were washed with brine (5 mL), dried over anhydrous Na₂SO₄. After filtration, the filtrate was concentrated under reduced pressure. The residue was purified by silica gel column chromatography, eluted with CH₂Cl₂/THF (3:1) to afford N-[(2-hydroxyphenyl) methyl]prop-2-enamide (400 mg, 50.54% yield) as a light yellow oil.

Step 2: To a stirred solution of N-[(2-hydroxyphenyl) methyl]prop-2-enamide (400 mg, 2.257 mmol, 1 equiv) and 3-(fluorosulfonyl)-1,2-dimethylimidazol-1-ium triflate (1481.92 mg, 4.514 mmol, 2 equiv) in MeCN (10 mL) was added TEA (685.27 mg, 6.771 mmol, 3 equiv) dropwise at 0° C. The resulting mixture was stirred at room temperature for 1 h. The resulting mixture was concentrated under reduced pressure. The residue was purified by reversed-phase flash chromatography with the following conditions: column, C18 silica gel; mobile phase, MeCN in Water (0.1% FA), 10% to 50% gradient in 10 min; detector, UV 254 nm. This resulted in 2-(prop-2-enamidomethyl) phenyl sulfurofluoridate (110 mg, 18.8% yield, 99.9% purity) as a white solid. LCMS:(ES, m/z): [M+H]⁺=260.0.

¹H NMR (300 MHz, DMSO-d₆, ppm) δ 8.70 (s, 1H), 7.59 (t, J=4.4 Hz, 1H), 7.51 (dt, J=5.4, 4.3 Hz, 3H), 6.30 (dd, J=17.1, 10.0 Hz, 1H), 6.14 (dd, J=17.1, 2.3 Hz, 1H), 5.66 (dd, J=10.0, 2.3 Hz, 1H), 4.47 (d, J=5.8 Hz, 2H).

### Example 135. Synthesis of 2-((aminooxy)methyl)phenyl sulfurofluoridate hydrochloride (PFS-3-2)

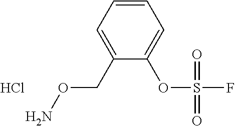 [SMILES: Cl ; NOCc1ccccc1OS(=O)(=O)F]

Step 1: To a stirred solution of saligenin (1 g, 8.055 mmol, 1 equiv), benzyl bromide (1.38 g, 8.055 mmol, 1 equiv) and K₂CO₃ (2.23 g, 16.110 mmol, 2 equiv) in MeCN (10 mL) at room temperature under nitrogen atmosphere. The resulting mixture was stirred at 60° C. for additional 2 h. The mixture was allowed to cool down to room temperature. The resulting mixture was diluted with 10 mL H₂O and extracted with EtOAc (10 mL×3). The combined organic layers were washed with brine (10 mL), dried over anhydrous Na₂SO₄. After filtration, the filtrate was concentrated under reduced pressure. The residue was purified by silica gel column chromatography, eluted with PE/EA (4:1) to afford [2-(benzyloxy) phenyl]methanol (1.1 g, 63.73% yield) as a light yellow oil.

Step 2: To a stirred solution of [2-(benzyloxy) phenyl]methanol (1.1 g, 5.134 mmol, 1 equiv) and PBr₃ (1.39 g, 5.134 mmol, 1 equiv) in DCM (15 mL) at room temperature under nitrogen atmosphere. The resulting mixture was stirred at room temperature for additional 3 h. The resulting mixture was diluted with 15 mL H₂O and extracted with DCM (15 mL×3). The combined organic layers were washed with brine (15 mL), dried over anhydrous Na₂SO₄. After filtration, the filtrate was concentrated under reduced pressure. The residue was purified by silica gel column chromatography, eluted with PE/THF (1:1) to afford 1-(benzyloxy)-2-(bromomethyl)benzene (500 mg, 35.14% yield) as a light yellow oil.

Step 3: To a stirred solution of 1-(benzyloxy)-2-(bromomethyl) benzene (500 mg, 1.804 mmol, 1 equiv) and tert-butyl N-hydroxycarbamate (288.24 mg, 2.165 mmol, 1.2 equiv) in MeCN (8 mL) were added DBU (411.97 mg, 2.706 mmol, 1.5 equiv) dropwise at 0° C. under nitrogen atmosphere. The resulting mixture was stirred at room temperature for additional 2 h. The resulting mixture was diluted with 8 mL H₂O and extracted with EtOAc (8 mL×3). The combined organic layers were washed with brine (8 mL), dried over anhydrous Na₂SO₄. After filtration, the filtrate was concentrated under reduced pressure. The residue was purified by silica gel column chromatography, eluted with PE/THF (2:1) to afford tert-butyl N-{[2-(benzyloxy)phenyl]methoxy}carbamate (550 mg, 92.56% yield) as a light yellow solid.

Step 4: To a stirred solution of tert-butyl N-{[2-(benzyloxy)phenyl]methoxy}carbamate (600 mg, 1.822 mmol, 1 equiv) and palladium (150 mg, 1.410 mmol, 0.77 equiv) in MeOH (6 mL) at room temperature under hydrogen atmosphere. The resulting mixture was stirred at room temperature for additional 2 h under hydrogen atmosphere. The resulting mixture was filtered, the filter cake was washed with MeOH (6 mL) (3 mL×3). The filtrate was concentrated under reduced pressure. This resulted in tert-butyl N-[(2-hydroxyphenyl) methoxy]carbamate (350 mg, 87.61% yield) as a yellow oil.

Step 5: To a stirred solution of tert-butyl N-[(2-hydroxyphenyl) methoxy]carbamate (350 mg, 1.463 mmol, 1 equiv) and 3-(fluorosulfonyl)-1,2-dimethylimidazol-1-ium triflate (720.23 mg, 2.195 mmol, 1.5 equiv) in MeCN (5 mL) were added TEA (296.05 mg, 2.926 mmol, 2 equiv) dropwise at 0° C. under nitrogen atmosphere. The resulting mixture was stirred at room temperature for additional 30 min. The resulting mixture was concentrated under reduced pressure. The residue was purified by reversed-phase flash chromatography with the following conditions: column, C18 silica gel; mobile phase, MeCN in Water (0.1% FA), 10% to 90% gradient in 10 min; detector, UV 254 nm. This resulted in tert-butyl N-({2-[(fluorosulfonyl) oxy]phenyl}methoxy) carbamate (200 mg, 42.55% yield) as a light yellow oil.

Step 6: To a stirred solution of tert-butyl N-({2-[(fluorosulfonyl) oxy]phenyl}methoxy) carbamate (200 mg, 0.622 mmol, 1 equiv) in DCM (2 mL) was added HCl in 1,4-dioxane (4.0 M) (2 mL, 0.055 mmol, 0.09 equiv) dropwise at room temperature under nitrogen atmosphere. The resulting mixture was stirred at room temperature for additional 2 h. The precipitated solids were collected by filtration and washed with diethyl ether (2 mL×2). The residue was purified by trituration with diethyl ether (3 mL). This resulted in 2-[(aminooxy) methyl]phenylsulfurofluoridate hydrochloride (95 mg, 59.24% yield, 96.3% purity) as a light yellow solid.

¹H NMR (300 MHz, DMSO-d₆, ppm) δ 10.98 (s, 2H), 7.85-7.50 (m, 4H), 5.17 (s, 2H).

### Example 136. Synthesis of 3-((aminooxy)methyl)phenyl sulfurofluoridate (PFS-4-2)

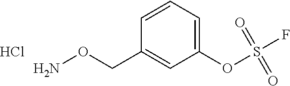 [SMILES: Cl ; NOCc1cccc(OS(=O)(=O)F)c1]

Step 1: Into a 40 mL sealed tube were added 1-(benzyloxy)-3-(bromomethyl)benzene (1.6 g, 5.77 mmol, 1.00 equiv), MeCN (20 mL) and tert-butyl N-hydroxycarbamate (0.92 g, 6.93 mmol, 1.20 equiv) at room temperature. To the above mixture was added DBU (0.88 g, 5.77 mmol, 1.00 equiv) dropwise over 2 min at 0° C. The resulting mixture was stirred at room temperature for additional overnight. The residue was purified by reversed-phase flash chromatography with the following conditions: column, C18 silica gel; mobile phase, MeCN in Water (0.1% NH₃·H₂O), 10% to 50% gradient in 10 min; detector, UV 254 nm. This resulted in tert-butyl N-{[3-(benzyloxy)phenyl]methoxy}carbamate (1.1 g, 57.85% yield, 95% purity) as a yellow oil.

Step 2: Into a 100 mL round-bottom flask were added tert-butyl N-{[3-(benzyloxy)phenyl]methoxy}carbamate (1.1 g, 3.34 mmol, 1.00 equiv), methanol (15 mL) and Pd/C (0.22 g, 2.07 mmol, 0.62 equiv) at room temperature. The resulting mixture was stirred at room temperature for 3 h under hydrogen atmosphere. The resulting mixture was filtered, the filter cake was washed with MeOH (3×3 mL). The filtrate was concentrated under reduced pressure. This resulted in tert-butyl N-[(3-hydroxyphenyl)methoxy]carbamate (800 mg) as a yellow oil.

Step 3: Into a 20 mL sealed tube were added tert-butyl N-[(3-hydroxyphenyl)methoxy]carbamate (800 mg, 3.34 mmol, 1.00 equiv), DCM (10 mL) and 1-(fluorosulfonyl)-2,3-dimethyl-1H-imidazol-3-ium trifluoromethanesulfonate (1.21 g, 3.68 mmol, 1.10 equiv) at room temperature. To the above mixture was added Et₃N (676.68 mg, 6.69 mmol, 2.00 equiv) dropwise over 2 min at −20° C. The resulting mixture was stirred at −20° C. for additional 1 h. The mixture was acidified to pH 5 with citric acid. The resulting mixture was extracted with CH₂Cl₂ (2×10 mL). The combined organic layers were washed with brine (1×10 mL), dried over anhydrous Na₂SO₄. After filtration, the filtrate was concentrated under reduced pressure. The residue was purified by reversed-phase flash chromatography with the following conditions: column, C18 silica gel; mobile phase, MeCN in Water (0.1% FA), 10% to 50% gradient in 10 min; detector, UV 254 nm. This resulted in tert-butyl N-({3-[(fluorosulfonyl)oxy]phenyl}methoxy)carbamate (500 mg, 46.54% yield, 98% purity) as a yellow oil.

Step 4: Into a 40 mL sealed tube were added tert-butyl N-({3-[(fluorosulfonyl)oxy]phenyl}methoxy)carbamate (250 mg, 0.79 mmol, 1.00 equiv) and hydrogen chloride (2.0 M in diethyl ether) (10 mL) at room temperature. The resulting mixture was stirred at room temperature for overnight under nitrogen atmosphere. The precipitated solids were collected by filtration and washed with diethyl ether (3×3 mL). This resulted in 3-[(aminooxy)methyl]phenyl sulfurofluoridate hydrochloride (69.7 mg, 34.77% yield, 95.9% purity) as a white solid. LCMS:(ES, m/z): [M+H]⁺=263.0.

¹H NMR-PH-TOPAZ-PFS-4-2-0 (300 MHz, DMSO-d₆, ppm) δ 11.08 (s, 3H), 7.73-7.54 (m, 4H), 5.17-5.08 (m, 2H).

### Example 137. Synthesis of 4-((aminooxy)methyl)phenyl sulfurofluoridate hydrochloride (PFS-5-1)

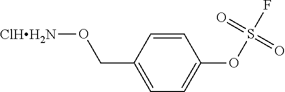

Step 1: To a stirred solution of 1-(benzyloxy)-4-(bromomethyl) benzene (1 g, 3.608 mmol, 1 equiv) and tert-butyl N-hydroxycarbamate (9.61 g, 72.160 mmol, 20 equiv) in ACN (20 mL) were added DBU (1.10 g, 7.216 mmol, 2 equiv) dropwise at 0° C. under nitrogen atmosphere. The resulting mixture was stirred at 40° C. for additional 2 h. The mixture was allowed to cool down to room temperature. The resulting mixture was diluted with 20 mL H₂O and extracted with EtOAc (20 mL×3). The combined organic layers were washed with brine (25 mL), dried over anhydrous Na₂SO₄. After filtration, the filtrate was concentrated under reduced pressure. The residue was purified by reversed-phase flash chromatography with the following conditions: column, C₁₈ silica gel; mobile phase, MeCN in Water (0.1% FA), 10% to 50% gradient in 10 min; detector, UV 254 nm This resulted in tert-butyl N-{[4-(benzyloxy) phenyl]methoxy}carbamate (1 g, 84.14% yield) as a light yellow oil.

Step 2: To a stirred solution of tert-butyl N-{[4-(benzyloxy) phenyl]methoxy}carbamate (1 g, 3.036 mmol, 1 equiv) and Pd/C (0.2 g, 1.879 mmol, 0.62 equiv) in MeOH (10 mL) at room temperature under hydrogen atmosphere. The resulting mixture was stirred at room temperature for additional 2 h under hydrogen atmosphere. The resulting mixture was filtered, the filter cake was washed with methanol (3×10 mL). The filtrate was concentrated under reduced pressure. The filtrate was concentrated under reduced pressure to afford tert-butyl N-[(4-hydroxyphenyl) methoxy]carbamate (900 mg, 123.90% yield) as a light yellow solid. The crude product was used in the next step directly without further purification.

Step 3: To a stirred solution of tert-butyl N-[(4-hydroxyphenyl) methoxy]carbamate (900 mg, 3.761 mmol, 1 equiv) and 3-(fluorosulfonyl)-1,2-dimethylimidazol-1-ium (1,1-difluoroethyl)(methylidene)-lambda6-sulfanoylolate (2424.77 mg, 7.522 mmol, 2 equiv) in MeCN (10 mL) were added TEA (1141.89 mg, 11.283 mmol, 3 equiv) dropwise at 0° C. under nitrogen atmosphere. The resulting mixture was stirred at room temperature for additional 1 h. The resulting mixture was concentrated under reduced pressure. The residue was purified by silica gel column chromatography, eluted with PE/THF (3:1) to afford tert-butyl N-({4-[(fluorosulfonyl) oxy]phenyl}methoxy) carbamate (400 mg, 33.10% yield) as a light yellow solid.

Step 4: To a stirred solution of tert-butyl N-({4-[(fluorosulfonyl) oxy]phenyl}methoxy) carbamate (400 mg, 1.245 mmol, 1 equiv) and hydrogen chloride (2.0 M in diethyl ether) (10 mL) at room temperature. The resulting mixture was stirred at room temperature for overnight under nitrogen atmosphere. The precipitated solids were collected by filtration and washed with diethyl ether (3×5 mL). This resulted in 4-[(aminooxy) methyl]phenyl sulfurofluoridate hydrochloride (50 mg, 15.59% yield, 96.5% purity) as a colorless oil. LCMS:(ES, m/z): [M+ACN]⁺=263.0.

¹H NMR-PH-TOPAZ-PFS-5-1-0 (300 MHz, DMSO-d₆) δ 11.07 (s, 3H), 7.72-7.60 (m, 4H), 5.10 (s, 2H).

### Example 138. Synthesis of 3-(3-(aminooxy)cyclobutyl)phenyl sulfurofluoridate (PFS-6-2)

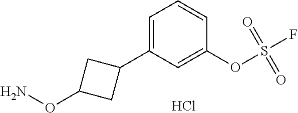 [SMILES: NOC1CC(c2cccc(OS(=O)(=O)F)c2)C1 ; Cl]

Step 1: Part A: To a stirred solution of [(3-iodocyclobutoxy)methyl]benzene (4.38 g, 15.201 mmol, 1 equiv), NiCl₂ (0.98 g, 7.601 mmol, 0.5 equiv) and 2,2′-Bipyridine (1.19 g, 7.601 mmol, 0.5 equiv) in DMF (20 mL) at room temperature under nitrogen atmosphere. The resulting mixture was stirred for 30 min at room temperature under nitrogen atmosphere. Part B: To a stirred mixture of 1-(benzyloxy)-3-bromobenzene (4 g, 15.201 mmol, 1 equiv) and NiCl₂ (0.98 g, 7.601 mmol, 0.5 equiv) in DMF (20 mL) at room temperature under nitrogen atmosphere. The resulting mixture was stirred for 30 min at room temperature under nitrogen atmosphere. The mixture of Part B was added to the mixture of Part A dropwise under nitrogen atmosphere. The resulting mixture was stirred for 16 h at room temperature under nitrogen atmosphere. The resulting mixture was diluted with 40 mL H₂O and extracted with EtOAc (40 mL×3). The combined organic layers were washed with brine (40 mL), dried over anhydrous Na₂SO₄. After filtration, the filtrate was concentrated under reduced pressure. The residue was purified by reversed-phase flash chromatography with the following conditions: column, C18 silica gel; mobile phase, MeCN in Water (0.1% FA), 10% to 50% gradient in 10 min; detector, UV 254 nm. This resulted in 1-(benzyloxy)-3-[3-(benzyloxy) cyclobutyl]benzene (1.5 g, 28.65% yield) as a light yellow oil.

Step 2: To a stirred solution of 1-(benzyloxy)-3-[3-(benzyloxy)cyclobutyl]benzene (1.5 g, 4.355 mmol, 1 equiv) and palladium (0.3 g, 2.819 mmol, 0.65 equiv) in MeOH (15 mL) at room temperature under hydrogen atmosphere. The resulting mixture was stirred at room temperature for additional 2 h under hydrogen atmosphere. The residue was washed with MeOH (5 mL×3). The resulting mixture was concentrated under reduced pressure. This resulted in 3-(3-hydroxycyclobutyl) phenol (750 mg, 104.89% yield) as a light yellow oil. The crude product was used in the next step directly without further purification.

Step 3: To a stirred solution of 3-(3-hydroxycyclobutyl)phenol (750 mg, 4.567 mmol, 1 equiv), benzyl bromide (781.21 mg, 4.567 mmol, 1 equiv) and K₂CO₃ (1262.50 mg, 9.134 mmol, 2 equiv) in MeCN (10 mL) at room temperature under nitrogen atmosphere. The resulting mixture was stirred at 60° C. for additional overnight. The mixture was allowed to cool down to room temperature. The resulting mixture was diluted with 10 mL H₂O and extracted with DCM (10 mL×3). The combined organic layers were washed with brine (10 mL), dried over anhydrous Na₂SO₄. After filtration, the filtrate was concentrated under reduced pressure. The residue was purified by silica gel column chromatography, eluted with PE/EA (5:1) to afford 3-[3-(benzyloxy) phenyl]cyclobutan-1-ol (500 mg, 43.04% yield) as a light yellow solid.

Step 4: To a stirred solution of 3-[3-(benzyloxy)phenyl]cyclobutan-1-ol (500 mg, 1.966 mmol, 1 equiv), N-hydroxyphthalimide (384.85 mg, 2.359 mmol, 1.2 equiv) and PPh3 (773.48 mg, 2.949 mmol, 1.5 equiv) in THF (10 mL) was added DIAD (596.30 mg, 2.949 mmol, 1.5 equiv) dropwise at 0° C. under nitrogen atmosphere. The resulting mixture was stirred at room temperature for additional 2 h. The resulting mixture was diluted with 10 mL H₂O and extracted with EtOAc (10 mL×3). The combined organic layers were washed with brine (10 mL), dried over anhydrous Na₂SO₄. After filtration, the filtrate was concentrated under reduced pressure. The residue was purified by silica gel column chromatography, eluted with PE/EA (3:1) to afford 2-{3-[3-(benzyloxy) phenyl]cyclobutoxy}isoindole-1,3-dione (520 mg, 66.22% yield) as a light yellow solid.

Step 5: To a stirred solution of 2-{3-[3-(benzyloxy)phenyl]cyclobutoxy}isoindole-1,3-dione (520 mg, 1.302 mmol, 1 equiv) in CHCl₃ (6 mL) and MeOH (2 mL) was added N₂H₄·H₂O (651.70 mg, 13.020 mmol, 10 equiv) dropwise at room temperature under nitrogen atmosphere. The resulting mixture was stirred at room temperature for additional 30 min. The resulting mixture was diluted with 8 mL H₂O and extracted with DCM (8 mL×3). The combined organic layers were washed with brine (8 mL), dried over anhydrous Na₂SO₄. After filtration, the filtrate was concentrated under reduced pressure. The crude product was used in the next step directly without further purification. To a stirred solution of the crude product and Boc₂O (568.23 mg, 2.604 mmol, 2 equiv) in THF (8 mL) was added TEA (65.87 mg, 0.651 mmol, 0.5 equiv) dropwise at 0° C. under nitrogen atmosphere. The resulting mixture was stirred at room temperature for additional overnight. The resulting mixture was diluted with 8 mL H₂O and extracted with EtOAc (8 mL×3). The combined organic layers were washed with brine (8 mL), dried over anhydrous Na₂SO₄. After filtration, the filtrate was concentrated under reduced pressure. The residue was purified by silica gel column chromatography, eluted with PE/THF (4:1) to afford tert-butyl N-{3-[3-(benzyloxy)phenyl]cyclobutoxy}carbamate (340 mg, 70.69% yield) as a light yellow oil.

Step 6: To a stirred solution of tert-butyl N-{3-[3-(benzyloxy) phenyl]cyclobutoxy}carbamate (340 mg, 0.920 mmol, 1 equiv) and palladium (50 mg, 0.470 mmol, 0.51 equiv) in MeOH (4 mL) at room temperature under hydrogen atmosphere. The resulting mixture was stirred at room temperature for additional 2 h under hydrogen atmosphere. The resulting mixture was filtered, the filter cake was washed with MeOH (2 mL×3). The filtrate was concentrated under reduced pressure. This resulted in tert-butyl N-[3-(3-hydroxyphenyl) cyclobutoxy]carbamate (200 mg, 77.80% yield) as a light yellow oil.

Step 7: To a stirred solution of tert-butyl N-[3-(3-hydroxyphenyl) cyclobutoxy]carbamate (200 mg, 0.716 mmol, 1 equiv) and 3-(fluorosulfonyl)-1,2-dimethylimidazol-1-ium triflate (352.53 mg, 1.074 mmol, 1.5 equiv) in MeCN (4 mL) was added TEA (144.91 mg, 1.432 mmol, 2 equiv) dropwise at 0° C. under nitrogen atmosphere. The resulting mixture was stirred at room temperature for additional 30 min. The resulting mixture was concentrated under reduced pressure. The residue was purified by reversed-phase flash chromatography with the following conditions: column, C18 silica gel; mobile phase, MeCN in Water (0.1% FA), 10% to 90% gradient in 10 min; detector, UV 254 nm. This resulted in tert-butyl N-(3-{3-[(fluorosulfonyl) oxy]phenyl}cyclobutoxy) carbamate (160 mg, 61.84% yield) as a light yellow solid.

Step 8: To a stirred solution of tert-butyl N-(3-{3-[(fluorosulfonyl) oxy]phenyl}cyclobutoxy) carbamate (160 mg, 0.443 mmol, 1 equiv) in DCM (1 mL) were added HCl in dioxane (1 mL) dropwise at room temperature under nitrogen atmosphere. The resulting mixture was stirred at room temperature for additional 2 h. The precipitated solids were collected by filtration and washed with diethyl ether (3×1 mL). The residue was purified by trituration with diethyl ether (2 mL). This resulted in 3-[3-(aminooxy) cyclobutyl]phenyl sulfurofluoridate hydrochloride (45 mg, 34.14% yield, 89.3% purity) as a light yellow solid. LCMS:(ES, m/z): [M+H]⁺=262.1.

¹H NMR (300 MHz, DMSO-d₆, ppm) δ 10.98 (s, 2H), 7.62-7.37 (m, 4H), 4.88-4.60 (m, 1H), 3.82-3.09 (m, 1H), 2.84-2.54 (m, 2H), 2.48-2.40 (m, 1H), 2.20-2.07 (m, 1H).

### Example 139. Synthesis of 4-(2-(aminooxy)ethoxy)phenyl sulfurofluoridate hydrochloride (PFS-7-1)

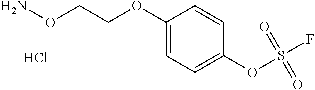 [SMILES: NOCCOc1ccc(OS(=O)(=O)F)cc1 ; Cl]

Step 1: Into a 40 mL sealed tube were added monobenzone (2 g, 9.988 mmol, 1.00 equiv) dibromoethane (1.88 g, 9.988 mmol, 1 equiv) and K2CO3 (2.76 g, 19.976 mmol, 2 equiv) at room temperature. The resulting mixture was stirred at room temperature for 3 h under nitrogen atmosphere. The resulting mixture was concentrated under vacuum. The residue was purified by silica gel column chromatography, eluted with PE/EA (1:1) to afford 1-(benzyloxy)-4-(2-bromoethoxy)benzene (1.9 g, 61.92% yield) as a yellow oil.

Step 2: To a stirred solution of tert-butyl N-hydroxycarbamate (8.24 g, 61.850 mmol, 10 equiv) and 1-(benzyloxy)-4-(2-bromoethoxy)benzene (1.9 g, 6.185 mmol, 1.00 equiv) in acetone was added DBU (1.88 g, 12.370 mmol, 2 equiv) dropwise/in portions at 0° C. under nitrogen atmosphere. The resulting mixture was stirred at 40° C. for additional 2 h. The mixture was allowed to cool down to room temperature. The resulting mixture was diluted with 20 mL H2O and extracted with EtOAc (20 mL×3). The combined organic layers were washed with brine (25 mL), dried over anhydrous Na2SO4. After filtration, the filtrate was concentrated under reduced pressure. The residue was purified by reversed-phase flash chromatography with the following conditions: column, C18 silica gel; mobile phase, MeCN in Water (0.1% FA), 10% to 50% gradient in 10 min; detector, UV 254 nm. This resulted in tert-butyl N-{2-[4-(benzyloxy)phenoxy]ethoxy}carbamate (1.2 g, 53.98% yield) as a yellow solid.

Step 3: Into a 100 mL round-bottom flask were added tert-butyl N-{2-[4-(benzyloxy)phenoxy]ethoxy}carbamate (1.2 g, 3.339 mmol, 1 equiv) and Pd/C (200 mg, 1.879 mmol, 0.56 equiv) at room temperature. The resulting mixture was stirred at room temperature for 2 h under hydrogen atmosphere. The resulting mixture was filtered, the filter cake was washed with methanol (3×5 mL). The filtrate was concentrated under reduced pressure. The crude product was used in the next step directly without further purification.

Step 4: To a stirred solution of tert-butyl N-[2-(4-hydroxyphenoxy)ethoxy]carbamate (800 mg, 2.971 mmol, 1.00 equiv) and 3-(fluorosulfonyl)-1,2-dimethylimidazol-1-ium triflate (1950.26 mg, 5.942 mmol, 2 equiv) in acetone was added TEA (901.84 mg, 8.913 mmol, 3 equiv) dropwise at 0° C. under nitrogen atmosphere. The resulting mixture was stirred at 0° C. for 30 min under nitrogen atmosphere. The residue was purified by silica gel column chromatography, eluted with PE/EA (1:1) to afford tert-butyl N-(2-{4-[(fluorosulfonyl)oxy]phenoxy}ethoxy)carbamate (500 mg, 47.90% yield) as a white solid.

Step 5: Into a 40 mL sealed tube were added tert-butyl N-(2-{4-[(fluorosulfonyl)oxy]phenoxy}ethoxy)carbamate (500 mg, 1.423 mmol, 1 equiv) and hydrogen chloride (2.0 M in diethyl ether) (10 mL) at room temperature. The resulting mixture was stirred at room temperature for overnight under nitrogen atmosphere. The precipitated solids were collected by filtration and washed with diethyl ether (3×5 mL). This resulted in 4-[2-(aminooxy)ethoxy]phenyl sulfurofluoridate (70 mg, 19.58% yield) as a white solid. LCMS:(ES, m/z): [M+H]⁺=252.0.

¹H NMR (300 MHz, DMSO-d₆) δ 11.00 (s, 2H), 7.60-7.50 (m, 2H), 7.20-7.07 (m, 2H), 4.60-4.04 (m, 4H).

### Example 140. Synthesis of 3-(4-(aminooxy)piperidin-1-yl)phenyl sulfurofluoridate (PFS-7-2)

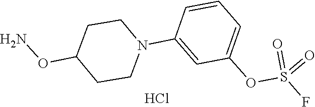 [SMILES: NOC1CCN(c2cccc(OS(=O)(=O)F)c2)CC1 ; Cl]

Step 1: To a stirred solution of N-hydroxyphthalimide (4 g, 24.520 mmol, 1 equiv), tert-butyl 4-hydroxypiperidine-1-carboxylate (5.92 g, 29.424 mmol, 1.2 equiv) and PPh₃ (9.65 g, 36.780 mmol, 1.5 equiv) in THF (50 mL) was added DIAD (7.44 g, 36.780 mmol, 1.5 equiv) dropwise at 0° C. under nitrogen atmosphere. The resulting mixture was stirred at room temperature for additional 2 h. The resulting mixture was diluted with 50 mL H₂O and extracted with EtOAc (50 mL×3). The combined organic layers were washed with brine (25 mL), dried over anhydrous Na₂SO₄. After filtration, the filtrate was concentrated under reduced pressure. The residue was purified by silica gel column chromatography, eluted with PE/EA (3:1) to afford tert-butyl 4-[(1,3-dioxoisoindol-2-yl)oxy]piperidine-1-carboxylate (3 g, 35.32% yield) as a yellow solid.

Step 2: To a stirred solution of tert-butyl 4-[(1,3-dioxoisoindol-2-yl)oxy]piperidine-1-carboxylate (2.7 g, 7.795 mmol, 1 equiv) in HCl in 1,4-dioxane (4.0 M) (10 mL) and DCM (10 mL) w at room temperature under nitrogen atmosphere. The resulting mixture was stirred at room temperature for additional 2 h. The precipitated solids were collected by filtration and washed with DCM (3×3 mL). This resulted in 2-(piperidin-4-yloxy)isoindole-1,3-dione (1.7 g, 88.56% yield) as a light yellow solid. The crude product was used in the next step directly without further purification.

Step 3: To a stirred solution of 2-(piperidin-4-yloxy)isoindole-1,3-dione (1.7 g, 6.903 mmol, 1 equiv), 1-(benzyloxy)-3-bromobenzene (2.18 g, 8.284 mmol, 1.2 equiv), Cs₂CO₃ (4.50 g, 13.806 mmol, 2 equiv), XPhos (0.66 g, 1.381 mmol, 0.2 equiv) and Pd₂(dba)₃ (0.63 g, 0.690 mmol, 0.1 equiv) in toluene (20 mL) at room temperature under nitrogen atmosphere. The resulting mixture was stirred at 100° C. for additional overnight. The mixture was allowed to cool down to room temperature. The resulting mixture was diluted with 20 mL H₂O and extracted with EtOAc (20 mL×3). The combined organic layers were washed with brine (20 mL), dried over anhydrous Na₂SO₄. After filtration, the filtrate was concentrated under reduced pressure. The residue was purified by silica gel column chromatography, eluted with PE/THF (3:1) to afford 2-({1-[3-(benzyloxy)phenyl]piperidin-4-yl}oxy)isoindole-1,3-dione (1.3 g, 43.95% yield) as a yellow solid.

Step 4: To a stirred solution of 2-({1-[3-(benzyloxy)phenyl]piperidin-4-yl}oxy)isoindole-1,3-dione (1.3 g, 3.034 mmol, 1 equiv) in CHCl₃ (9 mL) and MeOH (3 mL) were added N₂H₄·H₂O (1.52 g, 30.340 mmol, 10 equiv) dropwise at room temperature under nitrogen atmosphere. The resulting mixture was stirred at room temperature for additional 30 min. The resulting mixture was diluted with 10 mL H₂O and extracted with DCM (10 mL×3). The combined organic layers were washed with brine (10 mL), dried over anhydrous Na₂SO₄. After filtration, the filtrate was concentrated under reduced pressure. The crude product was used in the next step directly without further purification. To a stirred solution of the crude product and Boc₂O (1.32 g, 6.068 mmol, 2 equiv) in THF (15 mL) was added TEA (0.46 g, 4.551 mmol, 1.5 equiv) dropwise at 0° C. under nitrogen atmosphere. The resulting mixture was stirred at room temperature for additional overnight. The resulting mixture was diluted with 15 mL H₂O and extracted with EtOAc (15 mL×3). The combined organic layers were washed with brine (15 mL), dried over anhydrous Na₂SO₄. After filtration, the filtrate was concentrated under reduced pressure. The residue was purified by silica gel column chromatography, eluted with PE/THF (4:1) to afford tert-butyl N-({1-[3-(benzyloxy)phenyl]piperidin-4-yl}oxy)carbamate (700 mg, 57.90% yield) as a light yellow oil.

Step 5: To a stirred solution oftert-butyl N-({1-[3-(benzyloxy)phenyl]piperidin-4-yl}oxy)carbamate (700 mg, 1.757 mmol, 1 equiv) and palladium (100 mg, 0.940 mmol, 0.53 equiv) in MeOH (7 mL) at room temperature under hydrogen atmosphere. The resulting mixture was stirred at room temperature for additional 1 h. The resulting mixture was filtered, the filter cake was washed with MeOH (3×3 mL). The filtrate was concentrated under reduced pressure. This resulted in tert-butyl N-{[1-(3-hydroxyphenyl)piperidin-4-yl]oxy}carbamate (600 mg, 110.76% yield) as a light yellow oil. The crude product was used in the next step directly without further purification.

Step 6: To a stirred solution of tert-butyl N-{[1-(3-hydroxyphenylpiperidin-4-yl]oxy}carbamate (600 mg, 1.946 mmol, 1 equiv) and 3-(fluorosulfonyl)-1,2-dimethylimidazol-1-ium triflate (958.00 mg, 2.919 mmol, 1.5 equiv) in MeCN (10 mL) was added TEA (393.78 mg, 3.892 mmol, 2 equiv) dropwise at 0° C. under nitrogen atmosphere. The resulting mixture was stirred at room temperature for additional 30 min. The resulting mixture was concentrated under reduced pressure. The residue was purified by reversed-phase flash chromatography with the following conditions: column, C18 silica gel; mobile phase, MeCN in Water (0.1% FA), 10% to 90% gradient in 10 min; detector, UV 254 nm. This resulted in tert-butyl N-[(1-{3-[(fluorosulfonyl)oxy]phenyl}piperidin-4-yl)oxy]carbamate (200 mg, 26.33% yield) as a light yellow oil.

Step 7: To a stirred solution of tert-butyl N-[(1-{3-[(fluorosulfonyl)oxy]phenyl}piperidin-4-yl)oxy]carbamate (200 mg, 0.512 mmol, 1 equiv) in HCl in 1,4-dioxane (4.0 M) (1 mL) and DCM (1 mL) at room temperature under nitrogen atmosphere. The resulting mixture was stirred at room temperature for additional 1 h. The precipitated solids were collected by filtration and washed with diethyl ether (1 mL×3). The residue was purified by trituration with diethyl ether (2 mL). This resulted in 3-[4-(aminooxy)piperidin-1-yl]phenyl sulfurofluoridate (40 mg, 26.90% yield, 92.4% purity) as a light yellow solid. LCMS:(ES, m/z): [M+H]⁺=291.1. H NMR (300 MHz, DMSO-d₆, ppm) δ 11.10 (s, 2H), 7.38 (t, J=8.3 Hz, 1H), 7.17-7.04 (m, 2H), 6.88 (dd, J=7.8, 1.8 Hz, 1H), 4.34 (tt, J=8.1, 3.8 Hz, 1H), 3.60 (dt, J=11.1, 4.4 Hz, 2H), 3.09 (ddd, J=12.9, 9.1, 3.3 Hz, 2H), 2.15-1.98 (m, 2H), 1.65 (dtd, J=12.6, 8.7, 3.7 Hz, 2H).

### Example 141. Synthesis of 3-((2-(aminooxy)ethyl)carbamoyl)phenyl sulfurofluoridate (PFS-7-3)

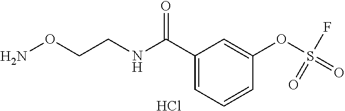 [SMILES: NOCCNC(=O)c1cccc(OS(=O)(=O)F)c1 ; Cl]

Step 1: To a stirred solution of N-(2-bromoethyl)phthalimide (5 g, 19.679 mmol, 1 equiv), tert-butyl N-hydroxycarbamate (3.93 g, 29.518 mmol, 1.5 equiv) and K₂CO₃ (5.44 g, 39.358 mmol, 2 equiv) in DMF (50 mL) at room temperature under nitrogen atmosphere. The resulting mixture was stirred at room temperature for additional 3 h. The resulting mixture was diluted with 50 mL H₂O and extracted with EtOAc (50 mL×3). The combined organic layers were washed with brine (50 mL), dried over anhydrous Na₂SO₄. After filtration, the filtrate was concentrated under reduced pressure. The residue was purified by silica gel column chromatography, eluted with PE/EA (4:1) to afford tert-butyl N-[2-(1,3-dioxoisoindol-2-yl) ethoxy]carbamate (6 g, 99.54% yield) as a light yellow solid.

Step 2: To a stirred solution of tert-butyl N-[2-(1,3-dioxoisoindol-2-yl)ethoxy]carbamate (6 g, 19.587 mmol, 1 equiv) in EtOH (60 mL) were added N₂H₄H₂O (4.90 g, 97.935 mmol, 5 equiv) dropwise at room temperature under nitrogen atmosphere. The resulting mixture was stirred at room temperature for additional 1 h. The resulting mixture was concentrated under reduced pressure. The residue was diluted with 30 mL H₂O and extracted with EtOAc (30 mL×3). The combined organic layers were washed with brine (30 mL), dried over anhydrous Na₂SO₄. After filtration, the filtrate was concentrated under reduced pressure. The residue was purified by silica gel column chromatography, eluted with PE/THF (7:1) to afford tert-butyl N-(2-aminoethoxy)carbamate (3.2 g, 92.71% yield) as a light yellow oil.

Step 3: To a stirred solution of tert-butyl N-(2-aminoethoxy)carbamate (1.5 g, 8.512 mmol, 1 equiv), HATU (4.86 g, 12.768 mmol, 1.5 equiv) and DIEA (2.20 g, 17.024 mmol, 2 equiv) in DMAc (20 mL) at room temperature under nitrogen atmosphere. The mixture was stirred for 20 min. 3-(benzyloxy) benzoic acid (2.91 g, 12.768 mmol, 1.5 equiv) was added stirred for 30 min. The resulting mixture was diluted with 20 mL H₂O and extracted with EtOAc (20 mL×3). The combined organic layers were washed with brine (20 mL), dried over anhydrous Na₂SO₄. After filtration, the filtrate was concentrated under reduced pressure. The residue was purified by silica gel column chromatography, eluted with PE/EA (2:1) to afford tert-butyl N-(2-{[3-(benzyloxy) phenyl]formamido}ethoxy)carbamate (1 g, 30.40% yield) as a light yellow oil.

Step 4: To a stirred solution of tert-butyl N-(2-{[3-(benzyloxy) phenyl]formamido}ethoxy)carbamate (1 g, 2.588 mmol, 1 equiv) and palladium (0.2 g, 1.879 mmol, 0.73 equiv) in MeOH (10 mL) at room temperature under hydrogen atmosphere. The resulting mixture was stirred at room temperature for additional 2 h under hydrogen atmosphere. The resulting mixture was filtered, the filter cake was washed with MeOH (5 mL×3). The filtrate was concentrated under reduced pressure. This resulted in tert-butyl N-{2-[(3-hydroxyphenyl) formamido]ethoxy}carbamate (600 mg, 78.25% yield) as a light yellow oil. The crude product was used in the next step directly without further purification.

Step 5: To a stirred solution of tert-butyl N-{2-[(3-hydroxyphenyl)formamido]ethoxy}carbamate (600 mg, 2.025 mmol, 1 equiv) and 3-(fluorosulfonyl)-1,2-dimethylimidazol-1-ium triflate (996.97 mg, 3.037 mmol, 1.5 equiv) in MeCN (10 mL) were added TEA (409.79 mg, 4.050 mmol, 2 equiv) dropwise at 0° C. under nitrogen atmosphere. The resulting mixture was stirred at room temperature for additional 30 min. The resulting mixture was concentrated under reduced pressure. The residue was purified by reversed-phase flash chromatography with the following conditions: column, C18 silica gel; mobile phase, MeCN in Water (0.1% FA), 10% to 90% gradient in 10 min; detector, UV 254 nm. This resulted in tert-butyl N-[2-({3-[(fluorosulfonyl)oxy]phenyl}formamido)ethoxy]carbamate (400 mg, 52.21% yield) as a light yellow oil.

Step 6: To a stirred solution of tert-butyl N-[2-({3-[(fluorosulfonyl)oxy]phenyl}formamido)ethoxy]carbamate (400 mg, 1.057 mmol, 1 equiv) in DCM (2 mL) were added HCl in 1,4-dioxane (4.0 M) (2 mL) dropwise at room temperature under nitrogen atmosphere. The resulting mixture was stirred at room temperature for additional 2 h. The precipitated solids were collected by filtration and washed with diethyl ether (3×3 mL). The residue was purified by trituration with diethyl ether (4 mL). This resulted in 3-{[2-(aminooxy)ethyl]carbamoyl}phenyl sulfurofluoridate hydrochloride (210 mg, 63.12% yield, 97.0% purity) as a light yellow solid. LCMS:(ES, m/z): [M+ACN+Na]=341.1.

¹H NMR (300 MHz, DMSO-d₆, ppm) δ 10.87 (s, 3H), 9.10 (s, 1H), 8.07 (dt, J=6.6, 1.4 Hz, 2H), 7.89-7.67 (m, 2H), 4.15 (t, J=5.3 Hz, 2H), 3.59-3.54 (m, 2H).

### Example 142. Synthesis of 3-(4-(aminooxy)piperidine-1-carbonyl)phenyl sulfurofluoridate hydrochloride (PFS-8-1)

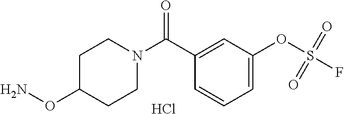 [SMILES: NOC1CCN(C(=O)c2cccc(OS(=O)(=O)F)c2)CC1 ; Cl]

Step 1: To a stirred solution of 3-(benzyloxy)benzoic acid (1 g, 4.381 mmol, 1 equiv), HATU (2.50 g, 6.572 mmol, 1.5 equiv) and DIEA (1.13 g, 8.762 mmol, 2 equiv) in DMF (15 mL) at room temperature under nitrogen atmosphere. The mixture was stirred for 20 min. piperidin-4-ol hydrobromide (1.20 g, 6.572 mmol, 1.5 equiv) was added and stirred for 1 h. The resulting mixture was diluted with 20 mL H₂O and extracted with EtOAc (20 mL×3). The combined organic layers were washed with brine (25 mL), dried over anhydrous Na₂SO₄. After filtration, the filtrate was concentrated under reduced pressure. This resulted in 1-[3-(benzyloxy) benzoyl]piperidin-4-ol (1.6 g, 117.28% yield) as a light yellow solid. The crude product/resulting mixture was used in the next step directly without further purification.

Step 2: To a stirred solution of 1-[3-(benzyloxy)benzoyl]piperidin-4-ol (1.6 g, 5.138 mmol, 1 equiv), N-hydroxyphthalimide (1.01 g, 6.166 mmol, 1.2 equiv) and PPH₃ (2.02 g, 7.707 mmol, 1.5 equiv) in THF (20 mL) were added DIAD (1.56 g, 7.707 mmol, 1.5 equiv) ropwise at 0° C. under nitrogen atmosphere. The resulting mixture was stirred at room temperature for additional 2 h. The resulting mixture was diluted with 20 mL H₂O and extracted with EtOAc (20 mL×3). The combined organic layers were washed with brine (25 mL), dried over anhydrous Na₂SO₄. After filtration, the filtrate was concentrated under reduced pressure. The residue was purified by silica gel column chromatography, eluted with PE/EA (2:1) to afford 2-({1-[3-(benzyloxy)benzoyl]piperidin-4-yl}oxy)isoindole-1,3-dione (1.8 g, 76.74% yield) as a light yellow solid.

Step 3: To a stirred solution of 2-({1-[3-(benzyloxy)benzoyl]piperidin-4-yl}oxy)isoindole-1,3-dione (1.8 g, 3.943 mmol, 1 equiv) in CHCl₃ (15 mL) and MeOH (5 mL) were added hydrazine hydrate (1.97 g, 39.430 mmol, 10 equiv) dropwise at room temperature under nitrogen atmosphere. The resulting mixture was stirred at room temperature for additional 30 min. The resulting mixture was diluted with 20 mL H₂O and extracted with DCM (20 mL×3). The combined organic layers were washed with brine (25 mL), dried over anhydrous Na₂SO₄. After filtration, the filtrate was concentrated under reduced pressure. The crude product was used in the next step directly without further purification. To a stirred solution of the crude product and Boc₂O (1.72 g, 7.886 mmol, 2 equiv) in THF (20 mL) was added TEA (0.60 g, 5.915 mmol, 1.5 equiv) dropwise at 0° C. under nitrogen atmosphere. The resulting mixture was stirred at room temperature for additional overnight. The resulting mixture was diluted with 20 mL H₂O and extracted with EtOAc (20 mL×3). The combined organic layers were washed with brine (25 mL), dried over anhydrous Na₂SO₄. After filtration, the filtrate was concentrated under reduced pressure. The resulting mixture was stirred at room temperature for additional overnight. The residue was purified by silica gel column chromatography, eluted with PE/THF (4:1) to afford tert-butyl N-({1-[3-(benzyloxy)benzoyl]piperidin-4-yl}oxy)carbamate (1.5 g, 89.19% yield) as a light yellow solid.

Step 4: To a stirred solution of tert-butyl N-({1-[3-(benzyloxy) benzoyl]piperidin-4-yl}oxy)carbamate (1.5 g, 3.517 mmol, 1 equiv) and palladium (0.3 g, 2.819 mmol, 0.80 equiv) in MeOH (15 mL) at room temperature under hydrogen atmosphere. The resulting mixture was stirred at room temperature for additional 2 h under hydrogen atmosphere. The resulting mixture was filtered, the filter cake was washed with MeOH (5 mL×3). The filtrate was concentrated under reduced pressure. This resulted in tert-butyl N-{[1-(3-hydroxybenzoyl)piperidin-4-yl]oxy}carbamate (1 g, 84.53% yield) as a light yellow oil. The crude product was used in the next step directly without further purification.

Step 5: To a stirred solution of tert-butyl N-{[1-(3-hydroxybenzoyl) piperidin-4-yl]oxy}carbamate (1 g, 2.973 mmol, 1 equiv) and 3-(fluorosulfonyl)-1,2-dimethylimidazol-1-ium triflate (1.46 g, 4.460 mmol, 1.5 equiv) in MeCN (10 mL) were added TEA (0.60 g, 5.946 mmol, 2 equiv) dropwise at 0° C. under nitrogen atmosphere. The resulting mixture was stirred at room temperature for additional 30 min. The resulting mixture was concentrated under reduced pressure. The residue was purified by reversed-phase flash chromatography with the following conditions: column, C18 silica gel; mobile phase, MeCN in Water (0.1% FA), 10% to 90% gradient in 10 min; detector, UV 254 nm. This resulted in tert-butyl N-[(1-{3-[(fluorosulfonyl)oxy]benzoyl}piperidin-4-yl)oxy]carbamate (600 mg, 48.23% yield) as a light yellow oil.

Step 6: To a stirred solution of tert-butyl N-[(1-{3-[(fluorosulfonyl)oxy]benzoyl}piperidin-4-yl)oxy]carbamate (600 mg, 1.434 mmol, 1 equiv) in DCM (3 mL) were added HCl in 1,4-dioxane (4.0 M) (3 mL) dropwise at room temperature under nitrogen atmosphere. The resulting mixture was stirred at room temperature for additional 2 h. The resulting mixture was filtered, the filter cake was washed with diethyl ether (3×3 mL). The filtrate was concentrated under reduced pressure. The residue was purified by trituration with diethyl ether (4 mL). This resulted in 3-[4-(aminooxy)piperidine-1-carbonyl]phenyl sulfurofluoridate hydrochloride (110 mg, 21.62% yield, 98.5% purity) as a white solid. LCMS:(ES, m/z): [M+H]⁺=319.1.

¹H NMR (400 MHz, DMSO-d₆, ppm) δ 10.93 (s, 3H), 7.75-7.64 (m, 3H), 7.57 (dt, J=6.7, 1.7 Hz, 1H), 4.35 (tt, J=7.7, 3.7 Hz, 1H), 3.90 (s, 1H), 3.53 (s, 2H), 3.18 (s, 1H), 1.99 (d, J=30.2 Hz, 2H), 1.67 (s, 2H).

### Example 143. Synthesis of 3-((3-(aminooxy)cyclobutyl)carbamoyl)phenyl sulfurofluoridate hydrochloride (PFS-8-2)

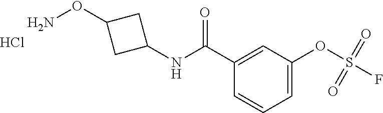 [SMILES: Cl ; NOC1CC(NC(=O)c2cccc(OS(=O)(=O)F)c2)C1]

Step 1: Into a 40 mL sealed tube were added tert-butyl N-(3-iodocyclobutyl)carbamate (2 g, 6.73 mmol, 1 equiv), DCM (10 mL) and HCl in 1,4-dioxane (4.0 M) (10 mL) at room temperature. The resulting mixture was stirred at room temperature for 2 h under nitrogen atmosphere. The resulting mixture was concentrated under reduced pressure. The residue was purified by trituration with dioxane (20 mL). The precipitated solids were collected by filtration and washed with 1,4-dioxane (3×5 mL). This resulted in 3-iodocyclobutan-1-amine (990 mg, 74.65% yield, 95% purity) as a white solid.

Step 2: Into a 40 mL sealed tube were added 3-(benzyloxy)benzoic acid (1.38 g, 6.03 mmol, 1.2 equiv), DMF (10 mL), HATU (2.87 g, 7.54 mmol, 1.5 equiv) and DIEA (1.30 g, 10.05 mmol, 2 equiv) at room temperature. The resulting mixture was stirred at room temperature for 15 min under nitrogen atmosphere. To the above mixture was added 3-iodocyclobutan-1-amine (0.99 g, 5.02 mmol, 1 equiv) in portions over 2 min at room temperature. The resulting mixture was stirred at room temperature for additional 2 h. The residue was purified by reversed-phase flash chromatography with the following conditions: column, C18 silica gel; mobile phase, MeCN in Water (0.1% NH3·H2O), 10% to 90% gradient in 10 min; detector, UV 254 nm. This resulted in 3-(benzyloxy)-N-(3-iodocyclobutyl)benzamide (1.2 g, 58.64% yield, 96% purity) as a white solid.

Step 3: Into a 40 mL sealed tube were added 3-(benzyloxy)-N-(3-iodocyclobutyl)benzamide (1.6 g, 3.93 mmol, 1 equiv), Cs2CO3 (3.84 g, 11.78 mmol, 3 equiv), MeCN (16 mL) and tert-butyl N-hydroxycarbamate (10.46 g, 78.58 mmol, 20 equiv) at room temperature. The resulting mixture was stirred at room temperature for overnight under nitrogen atmosphere. The resulting mixture was filtered, the filter cake was washed with MeCN (3×5 mL). The filtrate was concentrated under reduced pressure. The residue was purified by reversed-phase flash chromatography with the following conditions: column, C18 silica gel; mobile phase, MeCN in Water (0.1% NH3·H2O), 10% to 95% gradient in 10 min; detector, UV 254 nm. This resulted in tert-butyl N-{3-[3-(benzyloxy)benzamido]cyclobutoxy}carbamate (550 mg, 33.94% yield, 96% purity) as a white solid.

Step 4: Into a 100 mL round-bottom flask were added tert-butyl N-{3-[3-(benzyloxy)benzamido]cyclobutoxy}carbamate (550 mg, 1.33 mmol, 1 equiv), methanol (10 mL) and Pd/C (110 mg, 1.03 mmol, 0.78 equiv) at room temperature. The resulting mixture was stirred at room temperature for 2 h under hydrogen atmosphere. The resulting mixture was filtered, the filter cake was washed with methanol (3×5 mL). The filtrate was concentrated under reduced pressure. The crude product was used in the next step directly without further purification.

Step 5: Into a 40 mL sealed tube were added tert-butyl N-[3-(3-hydroxybenzamido)cyclobutoxy]carbamate (350 mg, 1.08 mmol, 1 equiv), MeCN (5 mL) and 3-(fluorosulfonyl)-1,2-dimethylimidazol-1-ium triflate (712.79 mg, 2.17 mmol, 2.0 equiv) at room temperature. To the above mixture was added Et3N (329.61 mg, 3.25 mmol, 3.0 equiv) dropwise over 5 min at 0° C. The resulting mixture was stirred at 0° C. for additional 1 h. The residue was purified by silica gel column chromatography, eluted with PE/THF (10:1) to afford tert-butyl N-(3-{3-[(fluorosulfonyl)oxy]benzamido}cyclobutoxy)carbamate (350 mg, 79.71% yield, 96% purity) as a yellow oil.

Step 6: Into a 40 mL sealed tube were added tert-butyl N-(3-{3-[(fluorosulfonyl)oxy]benzamido}cyclobutoxy)carbamate (200 mg, 0.49 mmol, 1 equiv) and hydrogen chloride (2.0 M in diethyl ether) (10 mL) at room temperature. The resulting mixture was stirred at room temperature for overnight under nitrogen atmosphere. The precipitated solids were collected by filtration and washed with diethyl ether (3×5 mL). This resulted in 3-{[3-(aminooxy)cyclobutyl]carbamoyl}phenyl sulfurofluoridate hydrochloride (51.3 mg, 30.44% yield, 99.1% purity) as a colorless solid. LCMS:(ES, m/z): [M+H]⁺=305.0.

¹H NMR (300 MHz, DMSO-d₆) δ 10.75 (s, 3H), 8.95 (d, J=7.3 Hz, 1H), 8.02 (td, J=3.6, 2.8, 1.6 Hz, 2H), 7.85-7.76 (m, 1H), 7.71 (t, J=8.2 Hz, 1H), 4.41 (p, J=7.2 Hz, 1H), 4.19-4.00 (m, 1H), 2.78-2.63 (m, 2H), 2.19 (tdd, J=9.2, 7.4, 2.9 Hz, 2H).

### Example 144. Synthesis of 4-((2-(aminooxy)ethyl)carbamoyl)phenyl sulfurofluoridate hydrochloride (PFS-8-3)

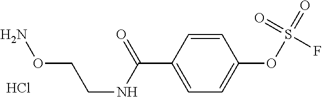 [SMILES: Cl ; NOCCNC(=O)c1ccc(OS(=O)(=O)F)cc1]

Step 1: To a stirred solution of tert-butyl N-(2-aminoethoxy) carbamate (1.74 g, 9.858 mmol, 1.5 equiv), DIEA (1.70 g, 13.144 mmol, 2 equiv) and HATU (3.75 g, 9.858 mmol, 1.5 equiv) in DMAc (20 mL) at room temperature under nitrogen atmosphere. The mixture was stirred for 15 min. 4-(benzyloxy)benzoic acid (1.5 g, 6.572 mmol, 1 equiv) was added and stirred for 1 h. The resulting mixture was diluted with 20 mL H₂O and extracted with EtOAc (20 mL×3). The combined organic layers were washed with brine (20 mL), dried over anhydrous Na₂SO₄. After filtration, the filtrate was concentrated under reduced pressure The residue was purified by silica gel column chromatography, eluted with PE/THF (2:1) to afford tert-butyl N-(2-{[4-(benzyloxy) phenyl]formamido}ethoxy)carbamate (1.1 g, 43.31% yield) as a light yellow oil.

Step 2: To a stirred solution of tert-butyl N-(2-{[4-(benzyloxy)phenyl]formamido}ethoxy)carbamate (1.1 g, 2.846 mmol, 1 equiv) and palladium (0.3 g, 2.819 mmol, 0.99 equiv) in MeOH (10 mL) at room temperature under hydrogen atmosphere. The resulting mixture was stirred at room temperature for additional 2 h under hydrogen atmosphere. The resulting mixture was filtered, the filter cake was washed with MeOH (5 mL×3). The filtrate was concentrated under reduced pressure. This resulted in tert-butyl N-{2-[(4-hydroxyphenyl)formamido]ethoxy}carbamate (600 mg, 71.14% yield) as a light yellow solid. The crude product was used in the next step directly without further purification.

Step 3: To a stirred solution of tert-butyl N-{2-[(4-hydroxyphenyl)formamido]ethoxy}carbamate (600 mg, 2.025 mmol, 1 equiv) and 3-(fluorosulfonyl)-1,2-dimethylimidazol-1-ium triflate (996.97 mg, 3.037 mmol, 1.5 equiv) in MeCN (10 mL) were added TEA (409.79 mg, 4.050 mmol, 2 equiv) dropwise at 0° C. under nitrogen atmosphere. The resulting mixture was stirred at room temperature for additional 30 min. The resulting mixture was concentrated under reduced pressure. The residue was purified by reversed-phase flash chromatography with the following conditions: column, C18 silica gel; mobile phase, MeCN in Water (0.1% FA), 10% to 90% gradient in 10 min; detector, UV 254 nm. This resulted in tert-butyl N-[2-({4-[(fluorosulfonyl)oxy]phenyl}formamido) ethoxy]carbamate (200 mg, 26.11% yield) as a light yellow oil.

Step 4: To a stirred solution of tert-butyl N-[2-({4-[(fluorosulfonyl)oxy]phenyl}formamido)ethoxy]carbamate (200 mg, 0.529 mmol, 1 equiv) in DCM (1 mL) were added tert-butyl N-[2-({4-[(fluorosulfonyl)oxy]phenyl}formamido) ethoxy]carbamate (200 mg, 0.529 mmol, 1 equiv) dropwise at room temperature under nitrogen atmosphere. The resulting mixture was stirred at room temperature for additional 2 h. The precipitated solids were collected by filtration and washed with diethyl ether (3×1 mL). The residue was purified by trituration with diethyl ether (2 mL). This resulted in 4-{[2-(aminooxy)ethyl]carbamoyl}phenyl sulfurofluoridate hydrochloride (44 mg, 26.45% yield, 90.7% purity) as a light yellow solid. LCMS:(ES, m/z): [M+H]⁺=279.1.

¹H NMR (300 MHz, DMSO-d₆, ppm) δ 10.7 (s, 1H), 9.10 (t, J=5.7 Hz, 1H), 8.22-8.03 (m, 2H), 7.73 (d, J=8.7 Hz, 2H), 4.12 (t, J=5.4 Hz, 2H), 3.55 (q, J=5.5 Hz, 2H).

### Example 145. Synthesis of N6-(3-((fluorosulfonyl)oxy)benzoyl)-L-lysine hydrochloride (FSK-02)

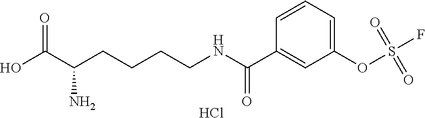 [SMILES: N[C@@H](CCCCNC(=O)c1cccc(OS(=O)(=O)F)c1)C(=O)O ; Cl]

Step 1: To a stirred solution of 3-[(fluorosulfonyl) oxy]benzoic acid (0.80 g, 3.638 mmol, 1.1 equiv), DIEA (1.28 g, 9.921 mmol, 3 equiv) and HATU (1.89 g, 4.960 mmol, 1.5 equiv) in DMF (40 mL) were added tert-butyl (2S)-6-amino-2-[(tert-butoxycarbonyl)amino]hexanoate (1 g, 3.307 mmol, 1 equiv) in portions at 0° C. The resulting mixture was stirred at 25° C. for additional 1 h. The reaction was monitored by LCMS. The resulting mixture was extracted with EtOAc (3×15 mL). The combined organic layers were washed with water (3×10 mL), dried over anhydrous Na₂SO₄. After filtration, the filtrate was concentrated under reduced pressure. The residue was purified by reversed-phase flash chromatography with the following conditions: column, C18 silica gel; mobile phase, MeCN in Water (0.1% FA), 10% to 70% gradient in 12 min; detector, UV 254 nm. This resulted in tert-butyl (2S)-2-[(tert-butoxycarbonyl) amino]-6-({3-[(fluorosulfonyl)oxy]phenyl}formamido)hexanoate (1.2 g, 71.92% yield) as a white solid.

Step 2: To a stirred mixture of tert-butyl (2S)-2-[(tert-butoxycarbonyl) amino]-6-({3-[(fluorosulfonyl)oxy]phenyl}formamido)hexanoate (1.2 g, 2.378 mmol, 1 equiv) was added HCl/dioxane 4M (20 mL) dropwise at 0° C. The resulting mixture was stirred at room temperature for additional 30 min. The residue was purified by trituration with MTBE (10 mL). This resulted in (2S)-2-amino-6-({3-[(fluorosulfonyl)oxy]phenyl}formamido) hexanoic acid hydrochloride (800 mg, 87.42% yield, 96.7% purity) as a white solid. LCMS: (ES, m/z): [M+H]⁺=349.1.

¹H NMR (300 MHz, DMSO-d₆, ppm) δ 13.76 (s, 1H), 8.82 (t, J=5.6 Hz, 1H), 8.38 (s, 2H), 8.05 (dd, J=7.5, 1.6 Hz, 2H), 7.85-7.66 (m, 2H), 3.88 (t, J=6.0 Hz, 1H), 3.29 (d, J=6.2 Hz, 2H), 1.83 (dt, J=9.7, 6.0 Hz, 2H), 1.66-1.52 (m, 2H), 1.51-1.29 (m, 2H).

### Example 146. Synthesis of 2-(methylsulfonyl)pyridin-4-yl sulfurofluoridate (SP-FS-1)

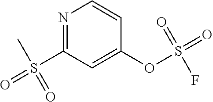

Step 1: To a stirred solution of 2-chloropyridin-4-ol (3 g, 23.159 mmol, 1 equiv) and K₂CO₃ (9.60 g, 69.477 mmol, 3 equiv) in DMSO (40 mL) was added [2-(chloromethoxy) ethyl]trimethylsilane (4.63 g, 27.791 mmol, 1.2 equiv) dropwise at 0° C. under nitrogen atmosphere. The resulting mixture was stirred at room temperature for additional 1 h. The resulting mixture was diluted with 40 mL H2O and extracted with EtOAc (40 mL×3). The combined organic layers were washed with brine (40 mL), dried over anhydrous Na₂SO₄. After filtration, the filtrate was concentrated under reduced pressure. The residue was purified by silica gel column chromatography, eluted with PE/THF (4/1) to afford 2-chloro-4-{[2-(trimethylsilyl) ethoxy]methoxy}pyridine (2.3 g, 38.23% yield) as a light yellow oil.

Step 2: To a stirred solution of 2-chloro-4-{[2-(trimethylsilyl) ethoxy]methoxy}pyridine (2.3 g, 8.853 mmol, 1 equiv) and sodium methanethiolate (0.50 g, 7.082 mmol, 0.8 equiv) in DMF (30 mL) at room temperature under nitrogen atmosphere. The resulting mixture was stirred at room temperature for additional 2 h. The resulting mixture was diluted with 30 mL H₂O and extracted with EtOAc (30 mL×3). The combined organic layers were washed with brine (30 mL), dried over anhydrous Na₂SO₄. After filtration, the filtrate was concentrated under reduced pressure. The residue was purified by reversed-phase flash chromatography with the following conditions: column, C18 silica gel; mobile phase, MeCN in Water (0.1% FA), 10% to 50% gradient in 10 min; detector, UV 254 nm. This resulted in 2-(methylsulfanyl)-4-{[2-(trimethylsilyl) ethoxy]methoxy}pyridine (1.2 g, 49.94% yield) as a light yellow oil.

Step 3: To a stirred solution of 2-(methylsulfanyl)-4-{[2-(trimethylsilyl) ethoxy]methoxy}pyridine (1.2 g, 4.421 mmol, 1 equiv) and TBAF (2.31 g, 8.842 mmol, 2 equiv) in THF (20 mL) at room temperature under nitrogen atmosphere. The resulting mixture was stirred at room temperature for additional 2 h. The resulting mixture was concentrated under reduced pressure. The residue was purified by reversed-phase flash chromatography with the following conditions: column, C18 silica gel; mobile phase, MeCN in Water (0.1% FA), 10% to 50% gradient in 10 min; detector, UV 254 nm. This resulted in 2-(methylsulfanyl) pyridin-4-ol (500 mg, 80.110% yield) as a light yellow oil.

Step 4: To a stirred solution of 2-(methylsulfanyl) pyridin-4-ol (500 mg, 3.541 mmol, 1 equiv) and 3-(fluorosulfonyl)-1,2-dimethylimidazol-1-ium triflate (2324.88 mg, 7.082 mmol, 2 equiv) in MeCN (10 mL) was added TEA (716.72 mg, 7.082 mmol, 2 equiv) dropwise at 0° C. under nitrogen atmosphere. The resulting mixture was stirred at room temperature for additional 30 min. The resulting mixture was concentrated under reduced pressure. The residue was purified by silica gel column chromatography, eluted with PE/THF (3:1) to afford 2-(methylsulfanyl) pyridin-4-yl sulfurofluoridate (120 mg, 15.18% yield) as a light yellow oil.

Step 5: To a stirred solution of 2-(methylsulfanyl) pyridin-4-yl sulfurofluoridate (120 mg, 0.538 mmol, 1 equiv) in DCM (2 mL) were added M-CPBA (185.52 mg, 1.076 mmol, 2 equiv) in portions at 0° C. The resulting mixture was stirred at 40° C. for additional 3 h. The mixture was allowed to cool down to room temperature. The resulting mixture was concentrated under reduced pressure. The residue was purified by reversed-phase flash chromatography with the following conditions: column, C18 silica gel; mobile phase, MeCN in Water (0.1% FA), 10% to 95% gradient in 10 min; detector, UV 254 nm. This resulted in 2-methanesulfonylpyridin-4-yl sulfurofluoridate (48 mg, 34.99% yield, 99.7% purity) as a light yellow oil. LCMS:(ES, m/z): [M+H]⁺=255.9.

¹H NMR (400 MHz, DMSO-d₆) δ 9.06 (d, J=5.5 Hz, 1H), 8.36 (d, J=2.4 Hz, 1H), 8.12 (ddd, J=5.5, 2.4, 1.0 Hz, 1H), 3.39 (s, 3H).

### Example 147. Synthesis of 6-(methylsulfonyl)pyridin-3-yl sulfurofluoridate (SP-FS-2)

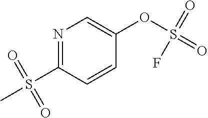

Step 1: To a stirred solution of 6-chloropyridin-3-ol (3 g, 23.159 mmol, 1 equiv) and K₂CO₃ (9.60 g, 69.477 mmol, 3 equiv) in DMSO (40 mL) was added [2-(chloromethoxy) ethyl]trimethylsilane (4.63 g, 27.791 mmol, 1.2 equiv) dropwise at 0° C. under nitrogen atmosphere. The resulting mixture was stirred at room temperature for additional 1 h. The resulting mixture was diluted with 40 mL H2O and extracted with EtOAc (40 mL×3). The combined organic layers were washed with brine (40 mL), dried over anhydrous Na₂SO₄. After filtration, the filtrate was concentrated under reduced pressure. The residue was purified by silica gel column chromatography, eluted with PE/THF (4/1) to afford 2-chloro-5-{[2-(trimethylsilyl) ethoxy]methoxy}pyridine (2.1 g, 34.90% yield) as a light yellow oil.

Step 2: To a stirred solution of 2-chloro-5-{[2-(trimethylsilyl) ethoxy]methoxy}pyridine (2.1 g, 8.083 mmol, 1 equiv) and Sodium thiomethoxide (0.45 g, 6.466 mmol, 0.8 equiv) in DMF (20 mL) at room temperature under nitrogen atmosphere. The resulting mixture was stirred at room temperature for additional 2 h. The resulting mixture was diluted with 30 mL H₂O and extracted with EtOAc (30 mL×3). The combined organic layers were washed with brine (30 mL), dried over anhydrous Na₂SO₄. After filtration, the filtrate was concentrated under reduced pressure. The residue was purified by reversed-phase flash chromatography with the following conditions: column, C18 silica gel; mobile phase, MeCN in Water (0.1% FA), 10% to 50% gradient in 10 min; detector, UV 254 nm. This resulted in 2-(methylsulfanyl)-5-{[2-(trimethylsilyl)ethoxy]methoxy}pyridine (1.1 g, 50.13% yield) as a light yellow oil.

Step 3: To a stirred solution of 2-(methylsulfanyl)-5-{[2-(trimethylsilyl) ethoxy]methoxy}pyridine (1.1 g, 4.052 mmol, 1 equiv) and tetrabutylazanium fluoride (2.56 g, 8.104 mmol, 2 equiv) in THF (20 mL) at room temperature under nitrogen atmosphere. The resulting mixture was stirred at room temperature for additional 2 h. The resulting mixture was concentrated under reduced pressure. The residue was purified by reversed-phase flash chromatography with the following conditions: column, C18 silica gel; mobile phase, MeCN in Water (0.1% FA), 10% to 50% gradient in 10 min; detector, UV 254 nm. This resulted in 6-(methylsulfanyl) pyridin-3-ol (500 mg, 87.39% yield) as a light yellow oil.

Step 4: To a stirred solution of 6-(methylsulfanyl) pyridin-3-ol (500 mg, 3.541 mmol, 1 equiv) and 3-(fluorosulfonyl)-1,2-dimethylimidazol-1-ium triflate (2324.88 mg, 7.082 mmol, 2 equiv) in MeCN (10 mL) were added TEA (716.72 mg, 7.082 mmol, 2 equiv) a dropwise at 0° C. under nitrogen atmosphere. The resulting mixture was stirred at room temperature for additional 30 min. The resulting mixture was concentrated under reduced pressure. The residue was purified by silica gel column chromatography, eluted with PE/THF (3:1) to afford 6-(methylsulfanyl) pyridin-3-yl sulfurofluoridate (250 mg, 31.62% yield) as a light yellow oil.

Step 5: To a stirred solution of 6-(methylsulfanyl) pyridin-3-yl sulfurofluoridate (250 mg, 1.120 mmol, 1 equiv) in DCM (2 mL) were added m-CPBA (386.49 mg, 2.240 mmol, 2 equiv) in portions at 0° C. under nitrogen atmosphere. The resulting mixture was stirred at 40° C. for additional 3 h. The mixture was allowed to cool down to room temperature. The resulting mixture was concentrated under reduced pressure. The residue was purified by reversed-phase flash chromatography with the following conditions: column, C18 silica gel; mobile phase, MeCN in Water (0.1% FA), 10% to 95% gradient in 10 min; detector, UV 254 nm. This resulted in 6-methanesulfonylpyridin-3-yl sulfurofluoridate (70 mg, 24.49% yield, 99.7% purity) as a light yellow oil. LCMS:(ES, m/z): [M+H]⁺=256.0.

¹H NMR (400 MHz, DMSO-d₆) δ 9.17 (d, J=2.7 Hz, 1H), 8.54 (dd, J=8.7, 2.7 Hz, 1H), 8.32 (d, J=8.7 Hz, 1H), 3.37 (s, 3H).

### Example 148. Synthesis of 6-(methylsulfonyl)pyridin-2-yl sulfurofluoridate (SP-FS-3)

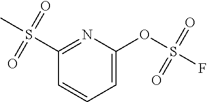

Step 1: To a stirred solution of 6-chloropyridin-2-ol (3 g, 23.15 mmol, 1 equiv) and K2CO3 (9.60 g, 69.47 mmol, 3 equiv) in DMSO (40 mL) were added SEMCI (4.63 g, 27.79 mmol, 1.2 equiv) in portions at 0° C. under nitrogen atmosphere. The resulting mixture was stirred at room temperature for additional 1 h. The resulting mixture was diluted with 40 mL H2O and extracted with EtOAc (40 mL×3). The combined organic layers were washed with brine (40 mL), dried over anhydrous Na2SO4. After filtration, the filtrate was concentrated under reduced pressure. The residue was purified by silica gel column chromatography, eluted with PE/THF (4/1) to afford 2-chloro-6-{[2-(trimethylsilyl) ethoxy]methoxy}pyridine (2 g, 33.24% yield) as a light yellow oil.

Step 2: To a stirred solution of 2-chloro-6-{[2-(trimethylsilyl) ethoxy]methoxy}pyridine (2.0 g, 7.69 mmol, 1 equiv) and Sodium thiomethoxide (95%) (0.43 g, 6.15 mmol, 0.8 equiv) in DMF (20 mL) at room temperature under nitrogen atmosphere. The resulting mixture was stirred at room temperature for additional 2 h. The resulting mixture was diluted with 30 mL H2O and extracted with EtOAc (30 mL×3). The combined organic layers were washed with brine (30 mL), dried over anhydrous Na2SO4. After filtration, the filtrate was concentrated under reduced pressure. The residue was purified by reversed-phase flash chromatography with the following conditions: column, C18 silica gel; mobile phase, MeCN in Water (0.1% FA), 10% to 50% gradient in 10 min; detector, UV 254 nm. This resulted in 2-(methylsulfanyl)-6-{[2-(trimethylsilyl) ethoxy]methoxy}pyridine (1.3 g, 62.21% yield) as a light yellow oil.

Step 3: To a stirred solution of 2-(methylsulfanyl)-6-{[2-(trimethylsilyl) ethoxy]methoxy}pyridine (1.3 g, 4.78 mmol, 1 equiv) and tetrabutylazanium fluoride (3.02 g, 9.57 mmol, 2 equiv) in THF (15 mL) at room temperature under nitrogen atmosphere. The resulting mixture was stirred at room temperature for additional 2 h. The residue was purified by reversed-phase flash chromatography with the following conditions: column, C18 silica gel; mobile phase, MeCN in Water (0.1% FA), 10% to 50% gradient in 10 min; detector, UV 254 nm. This resulted in 6-(methylsulfanyl) pyridin-2-ol (0.65 g, 96.13% yield) as a light yellow oil.

Step 4: To a stirred solution of 6-(methylsulfanyl) pyridin-2-ol (0.65 g, 4.60 mmol, 1 equiv) and 3-(fluorosulfinyl)-1,2-dimethylimidazol-1-ium difluoromethanesulfinate (1.92 g, 6.90 mmol, 1.5 equiv) in MeCN (10 mL) were added TEA (0.93 g, 9.20 mmol, 2 equiv) dropwise at 0° C. under nitrogen atmosphere. The resulting mixture was stirred at room temperature for additional 30 min. The resulting mixture was concentrated under reduced pressure. The residue was purified by silica gel column chromatography, eluted with PE/THF (3:1) to afford 6-(methylsulfanyl) pyridin-2-yl sulfurofluoridate (350 mg, 34.06% yield) as a light yellow oil.

Step 5: To a stirred solution of 6-(methylsulfanyl) pyridin-2-yl sulfurofluoridate (350 mg, 1.56 mmol, 1 equiv) in DCM (5 mL) were added M-CPBA (541.09 mg, 3.13 mmol, 2 equiv) in portions at 0° C. under nitrogen atmosphere. The resulting mixture was stirred at room temperature for additional 2 h. The resulting mixture was concentrated under reduced pressure. The residue was purified by reversed-phase flash chromatography with the following conditions: column, C18 silica gel; mobile phase, MeCN in Water (0.1% FA), 10% to 95% gradient in 10 min; detector, UV 254 nm. This resulted in 6-methanesulfonylpyridin-2-yl sulfurofluoridate (110 mg, 27.49% yield, 99.65% purity) as a light yellow oil. GCMS: GC-MS: (EI+, m/z, M⁺)=255.0.

¹H NMR (400 MHz, DMSO-d₆) δ 8.54 (t, J=7.9 Hz, 1H), 8.27 (d, J=7.6 Hz, 1H), 8.01 (d, J=8.2 Hz, 1H), 3.35 (s, 3H).

### Example 149. Synthesis of 3-(2-chloroacetyl)phenyl sulfurofluoridate (CAFS-3-A1)

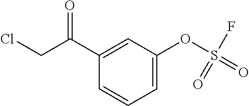 [SMILES: O=C(CCl)c1cccc(OS(=O)(=O)F)c1]

Step 1: A solution of 1-(3-hydroxyphenyl) ethan-1-one (500 mg, 3.67 mmol, 1 equiv) in MeCN (10 mL) was treated with 1-(fluorosulfonyl)-2,3-dimethyl-1H-imidazol-3-ium trifluoromethanesulfonate (1567.11 mg, 4.77 mmol, 1.3 equiv) at 0° C. for 3 min under nitrogen atmosphere followed by the addition of TEA (1114.87 mg, 11.01 mmol, 3 equiv) dropwise at 0° C. The resulting mixture was stirred at 0° C. for 1 h under nitrogen atmosphere. The resulting mixture was concentrated under reduced pressure. The residue was purified by silica gel column chromatography, eluted with PE/EA (5:1) to afford 3-acetylphenyl sulfonate (400 mg, 54.40% yield, 95% purity) as a yellow oil.

Step 2: A solution of 3-acetylphenyl sulfurofluoridate (400 mg, 1.83 mmol, 1 equiv) and benzyltrimethylazanium; dichloroiodanuide (1276.08 mg, 3.66 mmol, 2.0 equiv) in THF (5 mL) was stirred at room temperature for overnight under nitrogen atmosphere. The resulting mixture was concentrated under reduced pressure. The residue was purified by reversed-phase flash chromatography with the following conditions: column, C18 silica gel; mobile phase, MeCN in Water (0.1% NH₃*H₂O+10 mmol/L NH₄HCO₃), 10% to 50% gradient in 10 min; detector, UV 254 nm. This resulted in 3-(2-chloroacetyl) phenyl sulfurofluoridate (151.7 mg, 32.76% yield, 97.2% purity) as a yellow solid. LCMS:(ES, m/z): [M−H]⁻=250.9.

¹H NMR (300 MHz, DMSO-d₆, ppm) δ 8.19-8.05 (m, 2H), 8.00-7.90 (m, 1H), 7.80 (t, J=8.0 Hz, 1H), 5.26 (s, 2H).

### Example 150. Synthesis of 2-(methylsulfonyl)pyrimidin-5-yl sulfurofluoridate (FP-FS-1)

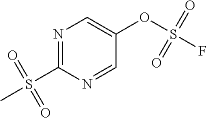

Step 1: To a stirred solution of 2-chloropyrimidin-5-ol (1 g, 7.661 mmol, 1 equiv) and Cs₂CO₃ (3.00 g, 9.193 mmol, 1.2 equiv) in DMF (25 mL) was added BnBr (3.93 g, 22.983 mmol, 3 equiv) in portions at room temperature under nitrogen atmosphere. The resulting mixture was stirred at room temperature for additional overnight. The reaction was monitored by LCMS. The resulting mixture was extracted with EtOAc (3×5 mL). The combined organic layers were washed with brine (3×5 mL), dried over anhydrous Na₂SO₄. After filtration, the filtrate was concentrated under reduced pressure. The residue was purified by Prep-TLC (PE/EA 5:1) to afford 5-(benzyloxy)-2-chloropyrimidine (750 mg, 44.37% yield) as a yellow solid.

Step 2: To a stirred solution of 5-(benzyloxy)-2-chloropyrimidine (750 mg, 3.399 mmol, 1 equiv) and sodium 2-sulfanylethane-1-sulfonate (558.00 mg, 3.399 mmol, 1 equiv) in DMF (5 mL) were at room temperature under nitrogen atmosphere. The resulting mixture was stirred at room temperature for additional 2 h. The resulting mixture was diluted with 5 mL H₂O and extracted with EtOAc (3×5 mL). The combined organic layer was washed with brine (5 mL), dried over anhydrous Na₂SO₄. After filtration, the filtrate was concentrated under reduced pressure. The residue was purified by silica gel column chromatography, eluted with PE/THF (4/1) to afford 5-(benzyloxy)-2-(methylsulfanyl)pyrimidine (500 mg, 63.33% yield) as a light yellow oil.

Step 3: To a stirred solution of 5-(benzyloxy)-2-(methylsulfanyl)pyrimidine (500 mg, 2.152 mmol, 1 equiv) in DCM (5 mL) were added Pd/C (229.06 mg, 2.152 mmol, 1 equiv) dropwise at 0° C. under nitrogen atmosphere. The resulting mixture was stirred at room temperature for additional 3 h. The resulting mixture was concentrated under reduced pressure. The residue was purified by reversed-phase flash chromatography with the following conditions: column, C18 silica gel; mobile phase, MeCN in Water (0.1% FA), 10% to 95% gradient in 10 min; detector, UV 254 nm. This resulted in 2-(methylsulfanyl)pyrimidin-5-ol (300 mg, 98.03% yield) as a white solid.

Step 4: To a stirred solution of 2-(methylsulfanyl)pyrimidin-5-ol (300 mg, 2.110 mmol, 1 equiv) and 3-(fluorosulfonyl)-1,2-dimethylimidazol-1-ium triflate (831.13 mg, 2.532 mmol, 1.2 equiv) in MeCN (5 mL) was added TEA (427.03 mg, 4.220 mmol, 2 equiv) dropwise at 0° C. The resulting mixture was stirred at room temperature for additional 1 h. The resulting mixture was concentrated under reduced pressure. The residue was purified by silica gel column chromatography, eluted with PE/THF (4/1) to afford 2-(methylsulfanyl)pyrimidin-5-yl sulfurofluoridate (100 mg, 21.14% yield) as a light yellow oil.

Step 5: To a stirred solution of 2-(methylsulfanyl) pyrimidin-5-yl sulfurofluoridate (100 mg, 0.446 mmol, 1 equiv) in DCM (3 mL) were added m-CPBA (153.92 mg, 0.892 mmol, 2 equiv) in portions at 0° C. under nitrogen atmosphere. The resulting mixture was stirred at 40° C. for additional 2 h. The mixture was allowed to cool down to room temperature. The resulting mixture was concentrated under reduced pressure. The residue was purified by reversed-phase flash chromatography with the following conditions: column, C18 silica gel; mobile phase, MeCN in Water (0.1% FA), 10% to 95% gradient in 10 min; detector, UV 254 nm. This resulted in 2-methanesulfonylpyrimidin-5-yl sulfurofluoridate (54 mg, 47.26% yield, 98.1% purity) as a light yellow yellow oil. GCMS: (ES, m/z): [M]⁺=255.9.

¹H NMR-PH-TOPAZ- FP-FS-1-0 (400 MHz, Chloroform-d, ppm) δ 9.03 (d, J=0.7 Hz, 2H), 3.45 (s, 3H).

### Example 151. Synthesis of N6-(4-((methylsulfonyl)oxy)benzoyl)-L-lysine hydrochloride (FSK-01)

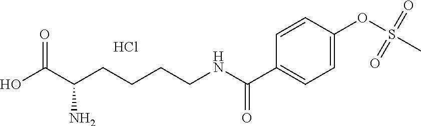 [SMILES: CS(=O)(=O)Oc1ccc(C(=O)NCCCC[C@H](N)C(=O)O)cc1 ; Cl]

Step 1: A mixture of 4-(benzyloxy)benzoic acid (5 g, 21.906 mmol, 1 equiv), DIEA (8.49 g, 65.688 mmol, 3 equiv) and HATU (12.49 g, 32.848 mmol, 1.5 equiv) in DMF (100 mL) was stirred at room temperature for 30 min. To the above mixture was added tert-butyl (2S)-6-amino-2-[(tert-butoxycarbonyl)amino]hexanoate (7.95 g, 26.287 mmol, 1.2 equiv) in portions at room temperature. The resulting mixture was stirred at room temperature for additional 1 h. The resulting mixture was extracted with CH₂Cl₂ (3×100 mL). The combined organic layers were washed with water (3×100 mL), dried over anhydrous Na₂SO₄. After filtration, the filtrate was concentrated under reduced pressure. The residue was purified by reversed-phase flash chromatography with the following conditions: column, C18 silica gel; mobile phase, MeCN in Water (0.1% FA), 10% to 95% gradient in 10 min; detector, UV 254 nm. This resulted in tert-butyl (2S)-6-{[4-(benzyloxy)phenyl]formamido}-2-[(tert-butoxycarbonyl)amino]hexanoate (6 g, 53.43% yield) as a white solid.

Step 2: To a solution of isopropyl (2S)-6-{[4-(benzyloxy)phenyl]formamido}-2-[(tert-butoxycarbonyl)amino]hexanoate (5 g, 10.028 mmol, 1 equiv) in 20 mL MeOH was added Pd/C (10%, 100 mg) under nitrogen atmosphere in a 40 mL round-bottom flask. The mixture was hydrogenated at room temperature for 2 h under hydrogen atmosphere using a hydrogen balloon, filtered through a Celite pad and concentrated under reduced pressure to afford tert-butyl (2S)-2-[(tert-butoxycarbonyl)amino]-6-[(4-hydroxyphenyl)formamido]hexanoate (4 g, 94.41% yield) as a black oil. The crude resulting mixture was used in the next step directly without further purification.

Step 3: To a stirred mixture of isopropyl (2S)-2-[(tert-butoxycarbonyl)amino]-6-[(4-hydroxyphenyl)formamido]hexanoate (4 g, 9.792 mmol, 1 equiv) and 3-(fluorosulfonyl)-1,2-dimethylimidazol-1-ium triflate (3.86 g, 11.750 mmol, 1.2 equiv) in MeCN (100 mL) was added TEA (1.49 g, 14.688 mmol, 1.5 equiv) in portions at 0° C. The resulting mixture was stirred at 0° C. for 30 min. The resulting mixture was concentrated under reduced pressure. The residue was purified by reversed-phase flash chromatography with the following conditions: column, C18 silica gel; mobile phase, MeCN in Water (0.1% FA), 10% to 95% gradient in 10 min; detector, UV 254 nm. This resulted in tert-butyl (2S)-2-[(tert-butoxycarbonyl)amino]-6-({4-[(fluorosulfonyl)oxy]phenyl}formamido)hexanoate (3 g, 60.72% yield) as a light yellow solid.

Step 4: Into a 40 mL sealed tube were added tert-butyl (2S)-2-[(tert-butoxycarbonyl)amino]-6-({4-[(fluorosulfonyl)oxy]phenyl}formamido)hexanoate (2 g, 3.964 mmol, 1 equiv) and hydrogen chloride (2.0 M in diethyl ether) (10 mL) at room temperature. The resulting mixture was stirred at room temperature for 24 h under nitrogen atmosphere. The precipitated solids were collected by filtration and washed with Et₂O (3×5 mL). This resulted in 2-amino-6-({4-[(fluorosulfonyl)oxy]phenyl}formamido)hexanoic acid (600 mg, 39.34% yield, 97.3% purity) as a light yellow solid. LCMS:(ES, m/z): [M+H]⁺=349.1.

¹H NMR (300 MHz, DMSO-d₆, ppm) 614.50-13.40 (s, 1H) 8.73 (t, J=5.6 Hz, 1H), 8.33 (s, 2H), 8.10-8.01 (m, 2H), 7.72 (d, J=8.5 Hz, 2H), 3.89 (t, J=6.1 Hz, 1H), 3.28 (d, J=6.2 Hz, 2H), 1.82 (d, J=5.7 Hz, 2H), 1.65-1.52 (m, 2H), 1.52-1.30 (m, 2H).

### Example 152. Preparation of Antigen-Binding Domains and Antigen-Binding Domain and Sample Preparation

SEQ Listing:
7D12 Native Sequence
SEQ ID NO: 1
QVKLEESGGGSVQTGGSLRLTCAASGRTSRSYGMGWFRQAPGKEREFVSGISWRGDST
GYADSVKGRFTISRDNAKNTVDLQMNSLKPEDTAIYYCAAAAGSAWYGTLYEYDYWG
QGTQVTVSSHHHHHH
7D12 - S31C
SEQ ID NO: 2
QVKLEESGGGSVQTGGSLRLTCAASGRTSRCYGMGWFRQAPGKEREFVSGISWRGDST
GYADSVKGRFTISRDNAKNTVDLQMNSLKPEDTAIYYCAAAAGSAWYGTLYEYDYWG
QGTQVTVSSHHHHHH
7D12 - Y109C
SEQ ID NO: 3
QVKLEESGGGSVQTGGSLRLTCAASGRTSRSYGMGWFRQAPGKEREFVSGISWRGDST
GYADSVKGRFTISRDNAKNTVDLQMNSLKPEDTAIYYCAAAAGSAWYGTLCEYDYWG
QGTQVTVSSHHHHHH
7D12-S31C
SEQ ID NO: 4
QVKLEESGGGSVQTGGSLRLTCAASGRTSRCYGMGWFRQAPGKEREFVSGISWRGDST
GYADSVKGRFTISRDNAKNTVDLQMNSLKPEDTAIYYCAAAAGSAWYGTLYEYDYWG
QGTQVTVSSHHHHHH
Y109C-SFY-Au-2-1
SEQ ID NO: 5
QVKLEESGGGSVQTGGSLRLTCAASGRTSRSYGMGWFRQAPGKEREFVSGISWRGDST
GYADSVKGRFTISRDNAKNTVDLQMNSLKPEDTAIYYCAAAAGSAWYGTL
Linker [SFY-Au-2-1]
SEQ ID NO: 46
EYDYWGQGTQVTVSSHHHHHH
Y109C-SFY-Au-3-1
SEQ ID NO: 6
QVKLEESGGGSVQTGGSLRLTCAASGRTSRSYGMGWFRQAPGKEREFVSGISWRGDST
GYADSVKGRFTISRDNAKNTVDLQMNSLKPEDTAIYYCAAAAGSAWYGTL
Linker [SFY-Au-3-1]
SEQ ID NO: 46
EYDYWGQGTQVTVSSHHHHHH
Y109C-SFY-4-1
SEQ ID NO: 7
QVKLEESGGGSVQTGGSLRLTCAASGRTSRSYGMGWFRQAPGKEREFVSGISWRGDST
GYADSVKGRFTISRDNAKNTVDLQMNSLKPEDTAIYYCAAAAGSAWYGTL
Linker [SFY-4-1]
SEQ ID NO: 46
EYDYWGQGTQVTVSSHHHHHH
Y109C-SFY-Au-4-2
SEQ ID NO: 8
QVKLEESGGGSVQTGGSLRLTCAASGRTSRSYGMGWFRQAPGKEREFVSGISWRGDST
GYADSVKGRFTISRDNAKNTVDLQMNSLKPEDTAIYYCAAAAGSAWYGTL
Linker [SFY-Au-4-2]
SEQ ID NO: 46
EYDYWGQGTQVTVSSHHHHHH
Y109C-SFY-Au-7-1
SEQ ID NO: 9
QVKLEESGGGSVQTGGSLRLTCAASGRTSRSYGMGWFRQAPGKEREFVSGISWRGDST
GYADSVKGRFTISRDNAKNTVDLQMNSLKPEDTAIYYCAAAAGSAWYGTL
Linker [SFY-Au-7-1]
SEQ ID NO: 46
EYDYWGQGTQVTVSSHHHHHH
Y109C-SFY-Au-8-1
SEQ ID NO: 10
QVKLEESGGGSVQTGGSLRLTCAASGRTSRSYGMGWFRQAPGKEREFVSGISWRGDST
GYADSVKGRFTISRDNAKNTVDLQMNSLKPEDTAIYYCAAAAGSAWYGTL
Linker [SFY-Au-8-1]
SEQ ID NO: 46
EYDYWGQGTQVTVSSHHHHHH
Y109C-SFY-Au-8-2
SEQ ID NO: 11
QVKLEESGGGSVQTGGSLRLTCAASGRTSRSYGMGWFRQAPGKEREFVSGISWRGDST
GYADSVKGRFTISRDNAKNTVDLQMNSLKPEDTAIYYCAAAAGSAWYGTL
Linker [SFY-Au-8-2]
SEQ ID NO: 46
EYDYWGQGTQVTVSSHHHHHH
Y109C-MFS-7-5
SEQ ID NO: 12
QVKLEESGGGSVQTGGSLRLTCAASGRTSRSYGMGWFRQAPGKEREFVSGISWRGDST
GYADSVKGRFTISRDNAKNTVDLQMNSLKPEDTAIYYCAAAAGSAWYGTL
Linker [MFS-7-5]
SEQ ID NO: 46
EYDYWGQGTQVTVSSHHHHHH
Y109C-BFS-3-1
SEQ ID NO: 13
QVKLEESGGGSVQTGGSLRLTCAASGRTSRSYGMGWFRQAPGKEREFVSGISWRGDST
GYADSVKGRFTISRDNAKNTVDLQMNSLKPEDTAIYYCAAAAGSAWYGTL
Linker [BAFS-3-1]
SEQ ID NO: 46
EYDYWGQGTQVTVSSHHHHHH
Y109C-BAFS-3-2
SEQ ID NO: 14
QVKLEESGGGSVQTGGSLRLTCAASGRTSRSYGMGWFRQAPGKEREFVSGISWRGDST
GYADSVKGRFTISRDNAKNTVDLQMNSLKPEDTAIYYCAAAAGSAWYGTL
Linker [BAFS-3-2]
SEQ ID NO: 46
EYDYWGQGTQVTVSSHHHHHH
Y109C-Au-1
SEQ ID NO: 15
QVKLEESGGGSVQTGGSLRLTCAASGRTSRSYGMGWFRQAPGKEREFVSGISWRGDST
GYADSVKGRFTISRDNAKNTVDLQMNSLKPEDTAIYYCAAAAGSAWYGTL
Linker [Au-1]
SEQ ID NO: 46
EYDYWGQGTQVTVSSHHHHHH
Y109C-Au-2
SEQ ID NO: 16
QVKLEESGGGSVQTGGSLRLTCAASGRTSRSYGMGWFRQAPGKEREFVSGISWRGDST
GYADSVKGRFTISRDNAKNTVDLQMNSLKPEDTAIYYCAAAAGSAWYGTL
Linker [Au-2]
SEQ ID NO: 46
EYDYWGQGTQVTVSSHHHHHH
Y109C-Au-3
SEQ ID NO: 17
QVKLEESGGGSVQTGGSLRLTCAASGRTSRSYGMGWFRQAPGKEREFVSGISWRGDST
GYADSVKGRFTISRDNAKNTVDLQMNSLKPEDTAIYYCAAAAGSAWYGTL
Linker [Au-3]
SEQ ID NO: 46
EYDYWGQGTQVTVSSHHHHHH
Y109C-Au-4
SEQ ID NO: 18
QVKLEESGGGSVQTGGSLRLTCAASGRTSRSYGMGWFRQAPGKEREFVSGISWRGDST
GYADSVKGRFTISRDNAKNTVDLQMNSLKPEDTAIYYCAAAAGSAWYGTL
Linker [Au-4]
SEQ ID NO: 46
EYDYWGQGTQVTVSSHHHHHH
Y109C-Au-5
SEQ ID NO: 19
QVKLEESGGGSVQTGGSLRLTCAASGRTSRSYGMGWFRQAPGKEREFVSGISWRGDST
GYADSVKGRFTISRDNAKNTVDLQMNSLKPEDTAIYYCAAAAGSAWYGTL
Linker [Au-5]
SEQ ID NO: 46
EYDYWGQGTQVTVSSHHHHHH
Y109C-MFS-3-3
SEQ ID NO: 20
QVKLEESGGGSVQTGGSLRLTCAASGRTSRSYGMGWFRQAPGKEREFVSGISWRGDST
GYADSVKGRFTISRDNAKNTVDLQMNSLKPEDTAIYYCAAAAGSAWYGTL
Linker [MFS-3-3]
SEQ ID NO: 46
EYDYWGQGTQVTVSSHHHHHH
Y109C-MFS-3-8
SEQ ID NO: 21
QVKLEESGGGSVQTGGSLRLTCAASGRTSRSYGMGWFRQAPGKEREFVSGISWRGDST
GYADSVKGRFTISRDNAKNTVDLQMNSLKPEDTAIYYCAAAAGSAWYGTL
Linker [MFS-3-8]
SEQ ID NO: 46
EYDYWGQGTQVTVSSHHHHHH
Y109C-MFS-3-16
SEQ ID NO: 22
QVKLEESGGGSVQTGGSLRLTCAASGRTSRSYGMGWFRQAPGKEREFVSGISWRGDST
GYADSVKGRFTISRDNAKNTVDLQMNSLKPEDTAIYYCAAAAGSAWYGTL
Linker [MFS-3-16]
SEQ ID NO: 46
EYDYWGQGTQVTVSSHHHHHH
Y109C-MFS-4-12
SEQ ID NO: 23
QVKLEESGGGSVQTGGSLRLTCAASGRTSRSYGMGWFRQAPGKEREFVSGISWRGDS
TGYADSVKGRFTISRDNAKNTVDLQMNSLKPEDTAIYYCAAAAGSAWYGTL
Linker [MFS-4-12]
SEQ ID NO: 46
EYDYWGQGTQVTVSSHHHHHH
S31C-MFS-8-4
SEQ ID NO: 24
QVKLEESGGGSVQTGGSLRLTCAASGRTSR
Linker [MFS-8-4]
SEQ ID NO: 47
YGMGWFRQAPGKEREFVSGISWRGDSTGYADSVKGRFTISRDNAKNTVDLQMNSLKPE
DTAIYYCAAAAGSAWYGTLYEYDYWGQGTQVTVSSHHHHHH
S31C-MFS-5-1
SEQ ID NO: 25
QVKLEESGGGSVQTGGSLRLTCAASGRTSR
Linker [MFS-5-1]
SEQ ID NO: 47
YGMGWFRQAPGKEREFVSGISWRGDSTGYADSVKGRFTISRDNAKNTVDLQMNSLKPE
DTAIYYCAAAAGSAWYGTLYEYDYWGQGTQVTVSSHHHHHH
S31C-MFS-8-7
SEQ ID NO: 26
QVKLEESGGGSVQTGGSLRLTCAASGRTSR
Linker [MFS-8-7]
SEQ ID NO: 47
YGMGWFRQAPGKEREFVSGISWRGDSTGYADSVKGRFTISRDNAKNTVDLQMNSLKPE
DTAIYYCAAAAGSAWYGTLYEYDYWGQGTQVTVSSHHHHHH
S31C-Au-PM-5
SEQ ID NO: 27
QVKLEESGGGSVQTGGSLRLTCAASGRTSR
Linker [Au-PM-5]
SEQ ID NO: 47
YGMGWFRQAPGKEREFVSGISWRGDSTGYADSVKGRFTISRDNAKNTVDLQMNSLKPE
DTAIYYCAAAAGSAWYGTLYEYDYWGQGTQVTVSSHHHHHH
S31C-Au-MP-6
SEQ ID NO: 28
QVKLEESGGGSVQTGGSLRLTCAASGRTSR
Linker [Au-MP-6]
SEQ ID NO: 47
YGMGWFRQAPGKEREFVSGISWRGDSTGYADSVKGRFTISRDNAKNTVDLQMNSLKPE
DTAIYYCAAAAGSAWYGTLYEYDYWGQGTQVTVSSHHHHHH
S31C-Au-PP-8
SEQ ID NO: 29
QVKLEESGGGSVQTGGSLRLTCAASGRTSR
Linker [Au-PP-8]
SEQ ID NO: 47
YGMGWFRQAPGKEREFVSGISWRGDSTGYADSVKGRFTISRDNAKNTVDLQMNSLKPE
DTAIYYCAAAAGSAWYGTLYEYDYWGQGTQVTVSSHHHHHH
S31C-Au-PM-4
SEQ ID NO: 30
QVKLEESGGGSVQTGGSLRLTCAASGRTSR
Linker [Au-PM-4]
SEQ ID NO: 47
YGMGWFRQAPGKEREFVSGISWRGDSTGYADSVKGRFTISRDNAKNTVDLQMNSLKPE
DTAIYYCAAAAGSAWYGTLYEYDYWGQGTQVTVSSHHHHHH
S31C-Au-MM-5
SEQ ID NO: 31
QVKLEESGGGSVQTGGSLRLTCAASGRTSR
Linker [Au-MM-5]
SEQ ID NO: 47
YGMGWFRQAPGKEREFVSGISWRGDSTGYADSVKGRFTISRDNAKNTVDLQMNSLKPE
DTAIYYCAAAAGSAWYGTLYEYDYWGQGTQVTVSSHHHHHH
S31C-Au-PM-6
SEQ ID NO: 32
QVKLEESGGGSVQTGGSLRLTCAASGRTSR
Linker [Au-PM-6]
SEQ ID NO: 47
YGMGWFRQAPGKEREFVSGISWRGDSTGYADSVKGRFTISRDNAKNTVDLQMNSLKPE
DTAIYYCAAAAGSAWYGTLYEYDYWGQGTQVTVSSHHHHHH
S31C-Au-MP-3
SEQ ID NO: 33
QVKLEESGGGSVQTGGSLRLTCAASGRTSR
Linker [Au-MP-3]
SEQ ID NO: 47
YGMGWFRQAPGKEREFVSGISWRGDSTGYADSVKGRFTISRDNAKNTVDLQMNSLKPE
DTAIYYCAAAAGSAWYGTLYEYDYWGQGTQVTVSSHHHHHH
S31C-Au-MP-7
SEQ ID NO: 34
QVKLEESGGGSVQTGGSLRLTCAASGRTSR
Linker [Au-MP-7]
SEQ ID NO: 47
YGMGWFRQAPGKEREFVSGISWRGDSTGYADSVKGRFTISRDNAKNTVDLQMNSLKPE
DTAIYYCAAAAGSAWYGTLYEYDYWGQGTQVTVSSHHHHHH
S31C-Au-PP-4
SEQ ID NO: 35
QVKLEESGGGSVQTGGSLRLTCAASGRTSR
Linker [Au-PP-4]
SEQ ID NO: 47
YGMGWFRQAPGKEREFVSGISWRGDSTGYADSVKGRFTISRDNAKNTVDLQMNSLKPE
DTAIYYCAAAAGSAWYGTLYEYDYWGQGTQVTVSSHHHHHH
S31C-Au-PP-5
SEQ ID NO: 36
QVKLEESGGGSVQTGGSLRLTCAASGRTSR
Linker [Au-PP-5]
SEQ ID NO: 47
YGMGWFRQAPGKEREFVSGISWRGDSTGYADSVKGRFTISRDNAKNTVDLQMNSLKPE
DTAIYYCAAAAGSAWYGTLYEYDYWGQGTQVTVSSHHHHHH
S31C-Au-PP-9
SEQ ID NO: 37
QVKLEESGGGSVQTGGSLRLTCAASGRTSR
Linker [Au-PP-9]
YGMGWFRQAPGKEREFVSGISWRGDSTGYADSVKGRFTISRDNAKNTVDLQMNSLKPE
DTAIYYCAAAAGSAWYGTLYEYDYWGQGTQVTVSSHHHHHH
S31C-SFY-Au-7-1
SEQ ID NO: 38
QVKLEESGGGSVQTGGSLRLTCAASGRTSR
Linker [SFY-Au-7-1]
SEQ ID NO: 47
YGMGWFRQAPGKEREFVSGISWRGDSTGYADSVKGRFTISRDNAKNTVDLQMNSLKPE
DTAIYYCAAAAGSAWYGTLYEYDYWGQGTQVTVSSHHHHHH
S31C-SFY-Au-8-1
SEQ ID NO: 39
QVKLEESGGGSVQTGGSLRLTCAASGRTSR
Linker [SFY-Au-8-1]
SEQ ID NO: 47
YGMGWFRQAPGKEREFVSGISWRGDSTGYADSVKGRFTISRDNAKNTVDLQMNSLKPE
DTAIYYCAAAAGSAWYGTLYEYDYWGQGTQVTVSSHHHHHH
S31C-SFY-Au-8-2
SEQ ID NO: 40
QVKLEESGGGSVQTGGSLRLTCAASGRTSR
Linker [SFY-Au-8-2]
SEQ ID NO: 47
YGMGWFRQAPGKEREFVSGISWRGDSTGYADSVKGRFTISRDNAKNTVDLQMNSLKPE
DTAIYYCAAAAGSAWYGTLYEYDYWGQGTQVTVSSHHHHHH
S31C-Au-MP-5
SEQ ID NO: 41
QVKLEESGGGSVQTGGSLRLTCAASGRTSR
Linker [Au-MP-5]
SEQ ID NO: 47
YGMGWFRQAPGKEREFVSGISWRGDSTGYADSVKGRFTISRDNAKNTVDLQMNSLKPE
DTAIYYCAAAAGSAWYGTLYEYDYWGQGTQVTVSSHHHHHH
S31C-Au-MP-8
SEQ ID NO: 42
QVKLEESGGGSVQTGGSLRLTCAASGRTSR
Linker [Au-MP-8]
SEQ ID NO: 47
YGMGWFRQAPGKEREFVSGISWRGDSTGYADSVKGRFTISRDNAKNTVDLQMNSLKPE
DTAIYYCAAAAGSAWYGTLYEYDYWGQGTQVTVSSHHHHHH
S31C-Au-MM-6
SEQ ID NO: 43
QVKLEESGGGSVQTGGSLRLTCAASGRTSR
Linker [Au-MM-6]
SEQ ID NO: 47
YGMGWFRQAPGKEREFVSGISWRGDSTGYADSVKGRFTISRDNAKNTVDLQMNSLKPE
DTAIYYCAAAAGSAWYGTLYEYDYWGQGTQVTVSSHHHHHH
S31C-Au-PM-7
SEQ ID NO: 44
QVKLEESGGGSVQTGGSLRLTCAASGRTSR
Linker [Au-PM-7]
SEQ ID NO: 47
YGMGWFRQAPGKEREFVSGISWRGDSTGYADSVKGRFTISRDNAKNTVDLQMNSLKPE
DTAIYYCAAAAGSAWYGTLYEYDYWGQGTQVTVSSHHHHHH
S31C-Au-PM-8
SEQ ID NO: 45
QVKLEESGGGSVQTGGSLRLTCAASGRTSR
Linker [Au-PM-8]
SEQ ID NO: 47
YGMGWFRQAPGKEREFVSGISWRGDSTGYADSVKGRFTISRDNAKNTVDLQMNSLKPE
DTAIYYCAAAAGSAWYGTLYEYDYWGQGTQVTVSSHHHHHH

For each of SEQ ID NOs: 5-45 shown above a first sequence is listed (e.g., SEQ ID NO: 5), followed by the specific linker which is linked to either SEQ ID NO: 46 or 47. Thus, for example Y109C-SFY-Au-2-1 is a construct including SEQ ID NO: 5, followed by linker [SFY-Au-2-1], and then SEQ ID NO: 46.

The antigen-binding domains described in examples 153 and 154 were prepared as follows:

### Protein Expression and Purification

The proteins described herein were expressed in both bacterial and mammalian cells.

The antigen-binding domains described herein were expressed in both bacterial and mammalian cells.

For bacterial expression, the antigen-binding domains were expressed in E. coli BL21 (DE3) cells using a pET26b plasmid encoding an N-terminal PelB signal peptide for periplasmic targeting and a C-terminal His-tag for purification. Cultures were grown in Terrific Broth (TB) and induced with IPTG to promote expression. After induction, cells were harvested and subjected to osmotic shock to extract proteins from the periplasmic space. The antigen-binding domains were then purified using nickel-affinity chromatography (Ni-NTA), followed by size exclusion chromatography using a column equilibrated with 50 mM Tris, 500 mM NaCl, 10% glycerol, 2 mM tris(2-carboxyethyl)phosphine (TCEP) and 5 mM ethylenediaminetetraacetic acid (EDTA) at pH 7.5. The final antigen-binding domains were concentrated using centrifugal filters with a 3 kDa molecular weight cutoff and stored in −80° C. until conjugation.

For mammalian expression, the genes coding for the antigen-binding domains were codon optimized for Cricetulus griseus and cloned into pcDNA3.4 (ThermoFisher Scientific) with a mouse immunoglobulin heavy chain signal peptide and C-terminal His-tag for purification. These antigen-binding domains were expressed by transient transfection of these plasmids into CHO—S cells. After culturing for three days post-transfection, the antigen-binding domains were purified from culture supernatants with magnetic nickel affinity chromatography resin, buffer exchanged into phosphate buffered saline (PBS) pH 7.2, concentrated to 0.8-1 mg/ml, and stored at −80° C. Prior to conjugation, mammalian produced antigen-binding domains were reduced by incubation with 5 molar equivalents of TCEP at 37° C. for 1.5-2 h.

Conjugation of 7D12-S31C (E. coli) with Maleimide Functionalized Linkers (MAL-Linkers)

The MAL-linkers described herein were freshly prepared as a 10 mM stock in DMSO (DMA, DMF or CH3CN are also acceptable), and 20-40 molar equivalents were added into a 1.5 mL tube containing 7D12-S31C (1.2 mg/mL, 0.5 mg in 50 mM Tris, 500 mM NaCl, 10% Glycerol, 2 mM TCEP, 5 mM EDTA buffer, pH 7.5). Additional DMSO was subsequently added as needed to adjust the final percentage of DMSO to 10%. The solution was then incubated for 1.5 hours at 25° C. or 30° C. and quenched with 40 molar equivalents of cysteine (141.4 μL, 10 mM stock in water). The reaction solution was purified via ultrafiltration using a 3 kDa MWCO centrifugal concentrator (Manufacturer: Sartorius, Volume: 4 mL) following manufacturer's instructions and using a buffer of 20 mM HEPES, 150 mM NaCl, 10% glycerol, pH 6.5. The identity of the final conjugate was confirmed via liquid chromatography-mass spectrometry (LC-MS) and its purity was evaluated by size exclusion high performance liquid chromatography (SEC-HPLC). Finally, the purified conjugate was frozen and stored in −80° C.

Table B below shows exemplary 7D12-S31C (E. Coli) malemide conjugates.

TABLE B

| Antigen- | | |
|---|---|---|
| Binding | | |
| Domain | Linker | Linker Structure |
| 7D12-S31C | NEM | 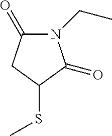 |
| 7D12-S31C | MFS-5.1 | 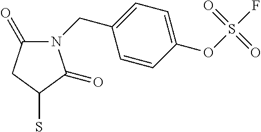 [SMILES: O=C1CC(S)C(=O)N1Cc1ccc(OS(=O)(=O)F)cc1] |
| 7D12-S31C | MFS-5.2 | 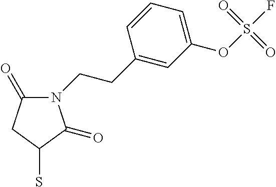 [SMILES: O=C1CC(S)C(=O)N1CCc1cccc(OS(=O)(=O)F)c1] |
| 7D12-S31C | MFS-6.1 | 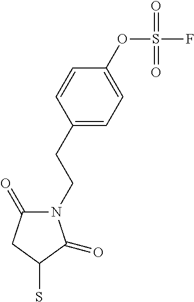 [SMILES: O=C1CC(S)C(=O)N1CCc1ccc(OS(=O)(=O)F)cc1] |
| 7D12-S31C | MFS-6.2 | 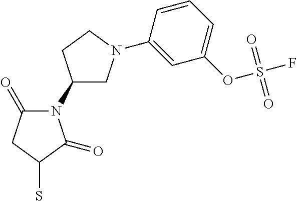 [SMILES: O=C1CC(S)C(=O)N1[C@H]1CCN(c2cccc(OS(=O)(=O)F)c2)C1] |
| 7D12-S31C | MFS-6.3 | 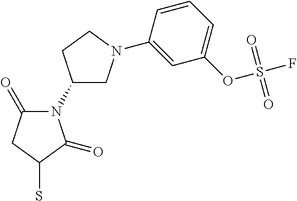 [SMILES: O=C1CC(S)C(=O)N1[C@@H]1CCN(c2cccc(OS(=O)(=O)F)c2)C1] |
| 7D12-S31C | MFS-7.4 | 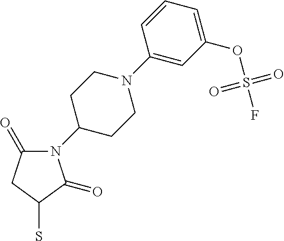 [SMILES: O=C1CC(S)C(=O)N1C1CCN(c2cccc(OS(=O)(=O)F)c2)CC1] |
| 7D12-S31C | MFS-8.2 | 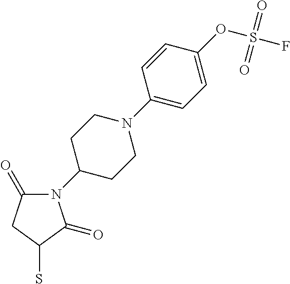 [SMILES: O=C1CC(S)C(=O)N1C1CCN(c2ccc(OS(=O)(=O)F)cc2)CC1] |
| 7D12-S31C | MFS-8.1 | 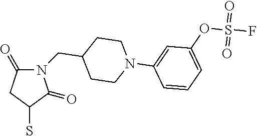 [SMILES: O=C1CC(S)C(=O)N1CC1CCN(c2cccc(OS(=O)(=O)F)c2)CC1] |
| 7D12-S31C | MFS-9.1 | 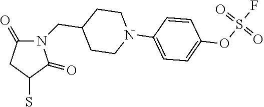 [SMILES: O=C1CC(S)C(=O)N1CC1CCN(c2ccc(OS(=O)(=O)F)cc2)CC1] |
| 7D12-S31C | MFS-7.5 | 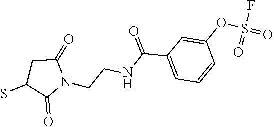 [SMILES: O=C(NCCN1C(=O)CC(S)C1=O)c1cccc(OS(=O)(=O)F)c1] |

Conjugation of 7D12-S31C (E. coli) with Bromo-Linkers (Br-Linkers)

The Br-linkers described herein were freshly prepared as a 10 mM stock in DMSO (DMA, DMF or CH3CN are also acceptable), and 20 molar equivalents were added into a 1.5 mL tube containing 7D12-S31C (1.3 mg/mL, 0.5 mg in 50 mM Tris, 500 mM NaCl, 10% Glycerol, 2 mM TCEP, 5 mM EDTA buffer, pH 7.5). Additional DMSO was subsequently added as needed to adjust the final percentage of DMSO to 10%. The solution was then incubated for 1.5 hours at 25° C. and quenched with 40 molar equivalents of cysteine (141.4 μL, 10 mM stock in water). The reaction solution was purified via ultrafiltration using a 3 kDa MWCO centrifugal concentrator (Manufacturer: Sartorius, Volume: 4 mL) following manufacturer's instructions and using a buffer of 20 mM HEPES, 150 mM NaCl, 10% glycerol, pH 6.5. The identity of the final conjugate was confirmed via liquid chromatography-mass spectrometry (LC-MS) and its purity was evaluated by size exclusion high performance liquid chromatography (SEC-HPLC). Finally, the purified conjugate was frozen and stored in −80° C.

Conjugation of 7D12-Y109C (E. coli) with Maleimide Functionalized Linkers (MAL-Linkers)

7D12-Y109C (Mammalian) (0.5 mg, 0.84 mg/mL, in PBS pH 7.2) was prepared for conjugation by dropwise addition of 5 molar equivalents of TCEP (50 mM stock in 100 mM HEPES buffer, pH 7.2) and incubation at 37° C. for 1.5 hours. The reduced protein was allowed to cool to 25° C. The MAL-linkers described herein were freshly prepared as a 10 mM stock in DMA (DMSO, DMF or CH3CN are also acceptable), and 10-20 molar equivalents were added into a 1.5 mL tube containing 7D12-Y109C (1.3 mg/mL, 0.5 mg in 50 mM Tris, 500 mM NaCl, 10% Glycerol, 2 mM TCEP, 5 mM EDTA buffer, pH 7.5). Additional DMA was subsequently added as needed to adjust the final percentage of DMA to 10%. The solution was then incubated for 1.5 hours at 25° C. and quenched with 40 molar equivalents of cysteine (141.4 μL, 10 mM stock in water). The reaction solution was purified via ultrafiltration using a 3 kDa MWCO centrifugal concentrator (Manufacturer: Sartorius, Volume: 4 mL) following manufacturer's instructions and using a buffer of 20 mM HEPES, 150 mM NaCl, 10% glycerol, pH 6.5. The identity of the final conjugate was confirmed via liquid chromatography-mass spectrometry (LC-MS) and its purity was evaluated by size exclusion high performance liquid chromatography (SEC-HPLC). Finally, the purified conjugate was frozen and stored in −80° C.

Conjugation of 7D12-Y109C (Mammalian) with Au-Linkers

7D12-Y109C (Mammalian) (0.5 mg, 0.84 mg/mL, in PBS pH 7.2) was prepared for conjugation by dropwise addition of 5 molar equivalents of TCEP (50 mM stock in 100 mM HEPES buffer, pH 7.2) and incubation at 37° C. for 1.5 hours. The reduced protein was allowed to cool to 25° C.

The Au-linkers described herein were freshly prepared as a 10 mM stock in CH3CN/water 1:1 (DMSO, DMF or DMA are also acceptable), and 5-10 molar equivalents were added into a 1.5 mL tube containing 7D12-Y109C (1.3 mg/mL, 0.5 mg in 200 mM Tris-HCl buffer, pH 7.0). The solution was then incubated for 1.5 hours at 25° C. and quenched with 40 molar equivalents of cysteine (141.4 μL, 10 mM stock in water). The reaction solution was purified via ultrafiltration using a 3 kDa MWCO centrifugal concentrator (Manufacturer: Sartorius, Volume: 4 mL) following manufacturer's instructions and using a buffer of 20 mM HEPES, 150 mM NaCl, 10% glycerol, pH 6.5. The identity of the final conjugate was confirmed via liquid chromatography-mass spectrometry (LC-MS) and its purity was evaluated by size exclusion high performance liquid chromatography (SEC-HPLC). Finally, the purified conjugate was frozen and stored in −80° C.

Conjugation of 7D12-Y109C (E. coli) with Bromo-Linkers (Br-Linkers)

The Br-linkers described herein were freshly prepared as a 10 mM stock in DMSO (DMA, DMF or CH3CN are also acceptable), and 20 molar equivalents were added into a 1.5 mL tube containing 7D12-Y109C (1.3 mg/mL, 0.5 mg in 50 mM Tris, 500 mM NaCl, 10% Glycerol, 2 mM TCEP, 5 mM EDTA buffer, pH 7.5). Additional DMSO was subsequently added as needed to adjust the final percentage of DMSO to 10%. The solution was then incubated for 1.5 hours at 25° C. and quenched with 40 molar equivalents of cysteine (141.4 μL, 10 mM stock in water). The reaction solution was purified via ultrafiltration using a 3 kDa MWCO centrifugal concentrator (Manufacturer: Sartorius, Volume: 4 mL) following manufacturer's instructions and using a buffer of 20 mM HEPES, 150 mM NaCl, 10% glycerol, pH 6.5. The identity of the final conjugate was confirmed via liquid chromatography-mass spectrometry (LC-MS) and its purity was evaluated by size exclusion high performance liquid chromatography (SEC-HPLC). Finally, the purified conjugate was frozen and stored in −80° C.

Conjugation of 7D12-S31C (Mammalian) with Maleimide Functionalized Linkers (MAL-Linkers)

7D12-S31C (0.5 mg, 0.84 mg/mL, in PBS pH 7.2) was prepared for conjugation by dropwise addition of 5 molar equivalents of TCEP (50 mM stock in 100 mM HEPES buffer, pH 7.2) and incubation at 37° C. for 1.5 hours. The reduced protein was allowed to cool to 25° C.

The MAL-linkers described herein were freshly prepared as a 10 mM stock in DMA (DMSO, DMF or CH3CN are also acceptable), and 3 molar equivalents were added into a 1.5 mL tube containing 0.5 mg of the reduced 7D12-S31C. Additional DMA was subsequently added as needed to adjust the final percentage of DMA to 10%. The solution was then incubated for 1.5 hours at 25° C. and quenched with 6 molar equivalents of cysteine (21 μL, 10 mM stock in water). The reaction solution was purified via ultrafiltration using a 3 kDa MWCO centrifugal concentrator (Manufacturer: Sartorius, Volume: 4 mL) following manufacturer's instructions and using a buffer of 20 mM HEPES, 150 mM NaCl, 10% glycerol, pH 6.5. The identity of the final conjugate was confirmed via liquid chromatography-mass spectrometry (LC-MS) and its purity was evaluated by size exclusion high performance liquid chromatography (SEC-HPLC). Finally, the purified conjugate was frozen and stored in −80° C.

Table C below shows exemplary 7D12-S31C malemide conjugates.

TABLE C

| | | |
|---|---|---|
| 7D12-S31C | MFS-4.4 | 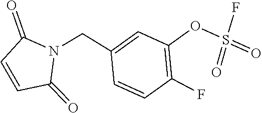 [SMILES: O=C1C=CC(=O)N1Cc1ccc(F)c(OS(=O)(=O)F)c1] |
| 7D12-S31C | MFS-4.12 | 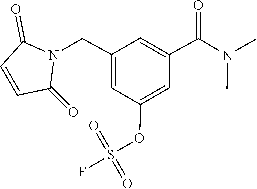 [SMILES: CN(C)C(=O)c1cc(CN2C(=O)C=CC2=O)cc(OS(=O)(=O)F)c1] |
| 7D12-S31C | MFS-7.5 | 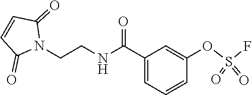 [SMILES: O=C(NCCN1C(=O)C=CC1=O)c1cccc(OS(=O)(=O)F)c1] |
| 7D12-S31C | MFS-8.3 | 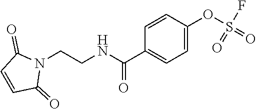 [SMILES: O=C(NCCN1C(=O)C=CC1=O)c1ccc(OS(=O)(=O)F)cc1] |
| 7D12-S31C | MFS-5.3 | 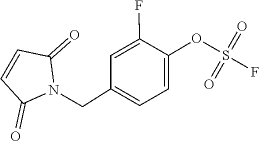 [SMILES: O=C1C=CC(=O)N1Cc1ccc(OS(=O)(=O)F)c(F)c1] |
| 7D12-S31C | MFS-8.4 | 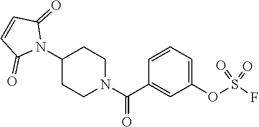 [SMILES: O=C(c1cccc(OS(=O)(=O)F)c1)N1CCC(N2C(=O)C=CC2=O)CC1] |

Conjugation of 7D12-S31C (Mammalian) with Au-Linkers

7D12-S31C (0.5 mg, 0.84 mg/mL, in PBS pH 7.2) was prepared for conjugation by dropwise addition of 5 molar equivalents of TCEP (50 mM stock in 100 mM HEPES buffer, pH 7.2) and incubation at 37° C. for 1.5 hours. The reduced protein was allowed to cool to 25° C.

The Au-linkers described herein or in table L were freshly prepared as a 10 mM stock in DMA (DMSO, DMF or CH3CN are also acceptable), and 5 molar equivalents were added into a 1.5 mL tube containing 0.5 mg of the reduced 7D12-S31C. Additional DMA was subsequently added as needed to adjust the final percentage of DMA to 10%. The solution was then incubated for 1.5 hours at 25° C. and quenched with 10 molar equivalents of cysteine (35 μL, 10 mM stock in water). The reaction solution was purified via ultrafiltration using a 3 kDa MWCO centrifugal concentrator (Manufacturer: Sartorius, Volume: 4 mL) following manufacturer's instructions and using a buffer of 20 mM HEPES, 150 mM NaCl, 10% glycerol, pH 6.5. The identity of the final conjugate was confirmed via liquid chromatography-mass spectrometry (LC-MS) and its purity was evaluated by size exclusion high performance liquid chromatography (SEC-HPLC). Finally, the purified conjugate was frozen and stored in −80° C.

Table D below shows 7D12-S31C VHH domains and linkers as described herein.

TABLE D

| Antigen-Binding | | |
|---|---|---|
| Domain | Linker | Linker Structure |
| 7D12-S31C | Au-4 | 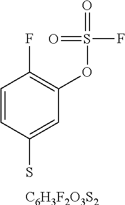 |
| 7D12-S31C | Au-5 | 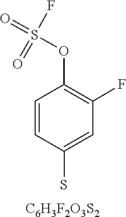 |
| 7D12-S31C | Au-MM-1 | 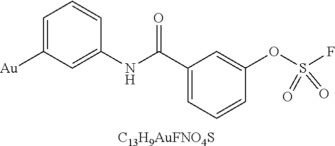 |
| 7D12-S31C | Au-PM-1 | 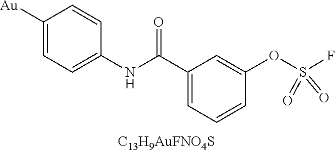 |
| 7D12-S31C | AU-MP-1 | 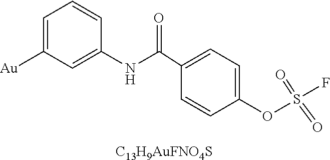 |
| 7D12-S31C | Au-PP-1 | 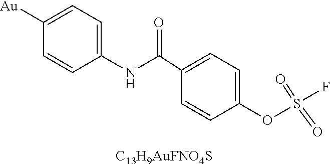 |
| 7D12-S31C | Au-MM-2 | 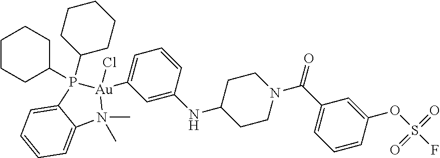 |
| | | C₁₃H₉AuFNO₄S |
| 7D12-S31C | Au-PM-2 | 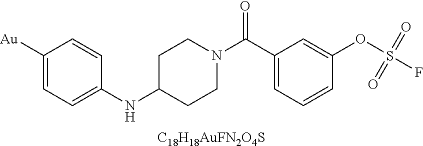 |

### Example 153. In Vitro Gel Shift Assays Demonstrating Covalency Between Antigen-Binding Domains and a Binding Target

Antigen-binding domains orbinders (e.g., VHH domains) were generated as follows: VHH sequences were cloned and expressed in Escherichia cohi with protein expression induced by isopropyl β-D-1-thiogalactopyranoside (TPTG). Following cell lysis, the periplasmic fraction was isolated, and VHHs were purified using immobilized metal affinity chromatography (IMAC). In some cases, VHHI constructs were also expressed in Chinese hamster ovary (CHO) cells. The secreted VHH from CHO cell culture supernatants were similarly purified by IMAC.

Binders of the present disclosure (e.g., “antigen-binding domains”) were tested for their binding affinity with the target (e.g., EGFR). In some examples, the binders were derived from a parent binder (i.e., 7D12-Y109C). For example, thebinders derived from 712-Y19C are shown in Table 1 and binding affinity was comparable across all conjugated 712-Y19C derivatives. Furthermore, kinetics were comparable across all conjugated samples.

TABLE 1

| Antigen- | | | | | |
|---|---|---|---|---|---|
| Binding | Plasmid | | KD | kon | koff |
| Domain | ID | Description | (nM) | (1/Ms) | (1/s) |
| Y109C- | 7D12- | MFS-3-1 | 31.9 | 1.62E+06 | 5.16E−02 |
| MFS-3-1 | Y109C | | | | |
| Y109C- | 7D12- | MFS-3-2 | 17.8 | 7.08E+05 | 1.26E−02 |
| MFS-3-2 | Y109C | | | | |
| Y109C- | 7D12- | MFS-4-1 | 26.8 | 1.73E+06 | 4.63E−02 |
| MFS-4-1 | Y109C | | | | |
| Y109C- | 7D12- | FP-FS-3-1 | 13.3 | 2.42E+06 | 3.22E−02 |
| FP-FS-1 | Y109C | | | | |
| Y109C- | 7D12- | NEM | 26.4 | 1.55E+06 | 4.08E−02 |
| NEM | Y109C | | | | |
| 7D12 | 7D12- | 7D12-Y109C | 117.0 | 1.52E+05 | 1.77E−02 |
| Y109C | Y109C | | | | |
| 7D12 wt | 7D12 wt | WT clone for | 1.0 | 3.20E+06 | 3.04E−03 |
| ″ | ″ | 7D12-S31C and | | | |
| | | 7D12-Y109C | | | |

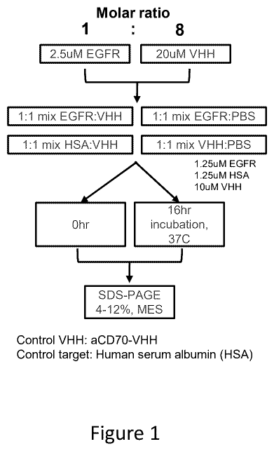

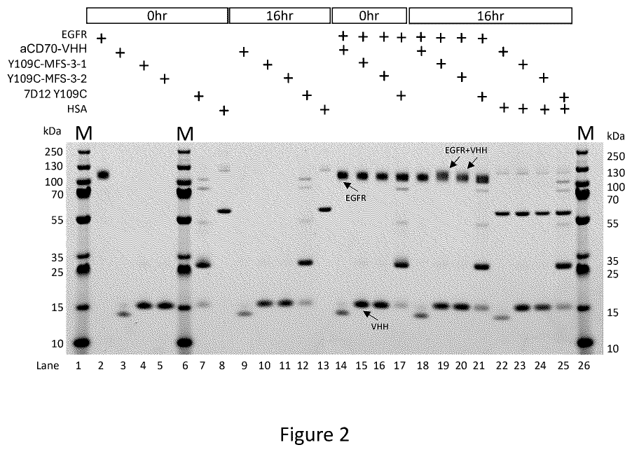

Next, a series of gel shift assays were performed to demonstrate conjugation between the antigen-binding domain and the target. FIG. 1 shows a crosslinking and gel shift protocol and FIG. 2 shows a gel shift assay after incubation between the antigen-binding domains shown and EGFR.

Briefly, 2.5 μM target (EGFR) and 20 μM binder (e.g., antigen-binding domain) were incubated according to the protocol shown in FIG. 2. The samples were then allowed to incubate overnight (16 hours) and run on an SDS-PAGE gel. Controls include a control binder: a CD70-VHH and human serum albumin (HAS). The data demonstrate a target:binder complex forms after overnight incubation. Specifically, the data demonstrate Y109C-MFS-3-1 and Y109C-MFS-3-2 crosslinked with EGFR after overnight incubation.

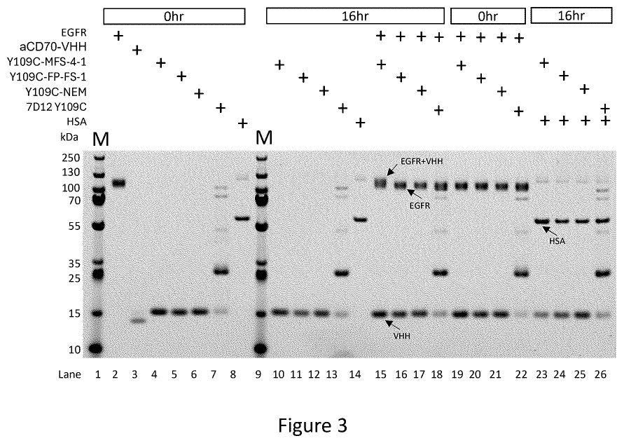

Additional binders were tested according to the protocol shown in FIG. 2. The data shown in FIG. 3 demonstrate a target:binder complex formed after overnight incubation. More specifically, Y109C-MFS-4-1 crosslinked with EGFR after overnight incubation.

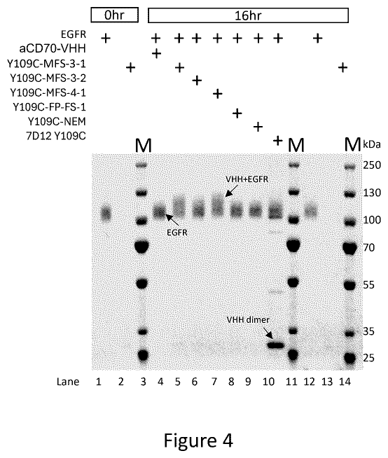

FIG. 4 is a gel shift assay performed according to the protocol shown in FIG. 2. While FIG. 4 shows a target:binder complex (e.g., EGFR:binder complex) for, Y109C-MFS-3-1, Y109C-MFS-3-2, and Y109C-MFS-4-1, a longer gel run time did not significantly improve resolution between the EGFR:binder complex and EGFR-only proteins.

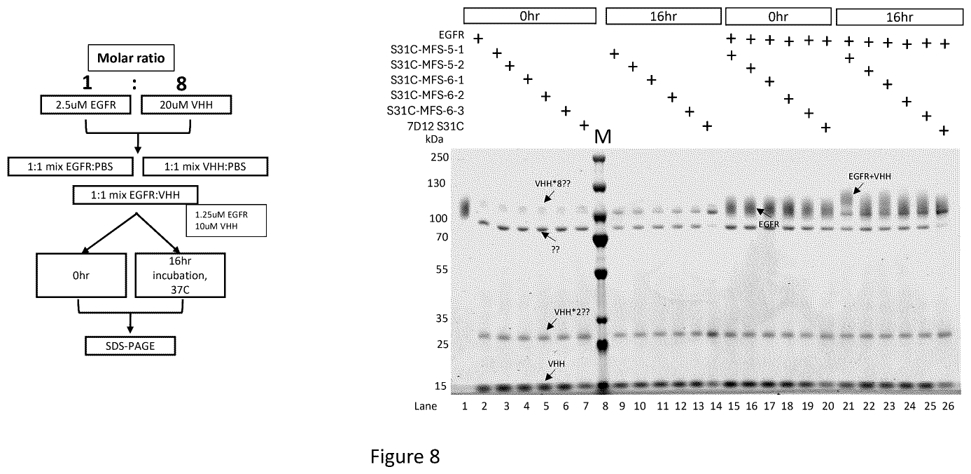

FIG. 8 shows a crosslinking and gel shift protocol (left) and a gel shift assay after incubation between the antigen-binding domains shown and EGFR. The binders shown in FIG. 8 were derived from 7D12 S31C.

Briefly, 2.5 μM of EGFR was incubated with 20 μM binder according to the protocol shown in FIG. 8 and allowed to incubate for 16 hours. The gel shift data shows that each of S31C-MFS-5-1, S31C-MFS-5-2, S31C-MFS-6-1, S31C-MFS-6-2, and S31C-MFS-6-3 all exhibit some degree of crosslinking with EGFR after overnight incubation.

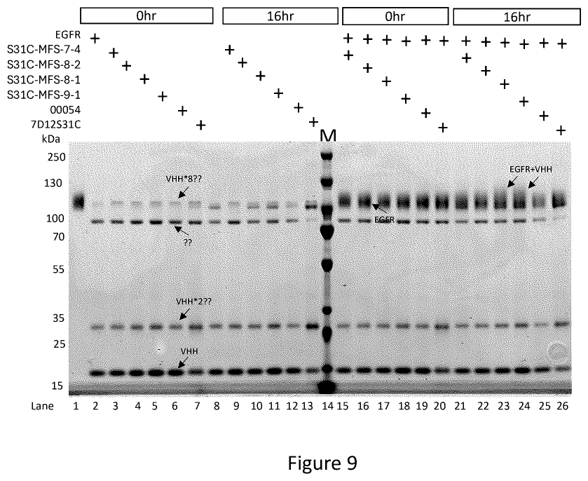

FIG. 9 shows the results of another crosslinking and gel shift assay performed with additional 7D12 S31C derived binders. The experiment as performed according to the protocol shown in FIG. 8 and the data demonstrate that S31C-MFS-8-1 and S31C-MFS-9-1 exhibit crosslinking with EGFR after overnight incubation.

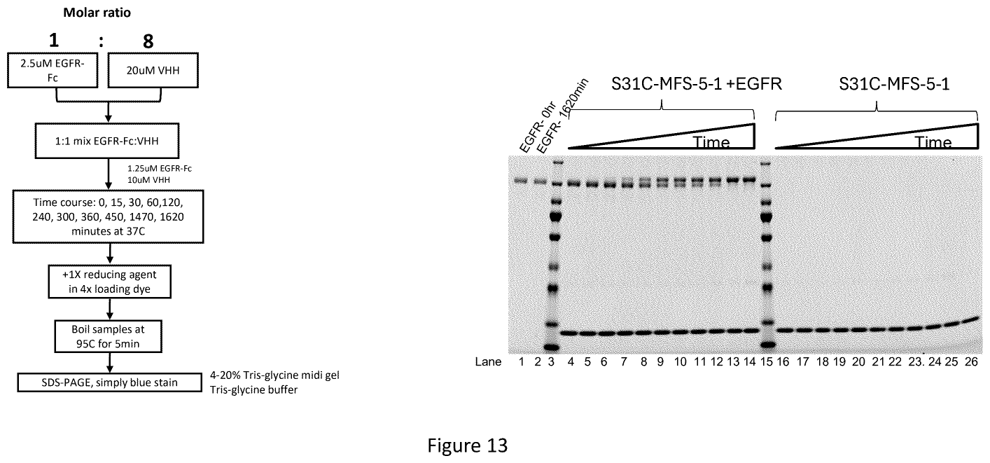

FIG. 13 is a crosslinking and gel shift protocol (left) and a gel shift assay (right) after incubation between S31C-MFS-5-1 and EGFR over time. Briefly, the crosslinking and gel shift was performed as shown including a time course (0, 15, 30, 60, 120, 240, 360, 450, 1470, and 1620 minutes) and boiling the samples at 95° C. for 5 minutes prior to the gel shift assay. The data demonstrate that S31C-MFS-5-1 form covalency over time with human (huEGFR). Moreover, EGFR: S31C-MFS-5-1 complex formation was clearly differentiated from EGFR-alone protein while boiling sample and/or with 4-20% Tris-glycine gel. The control gel on the right shows no complex formation in the absence of EGFR.

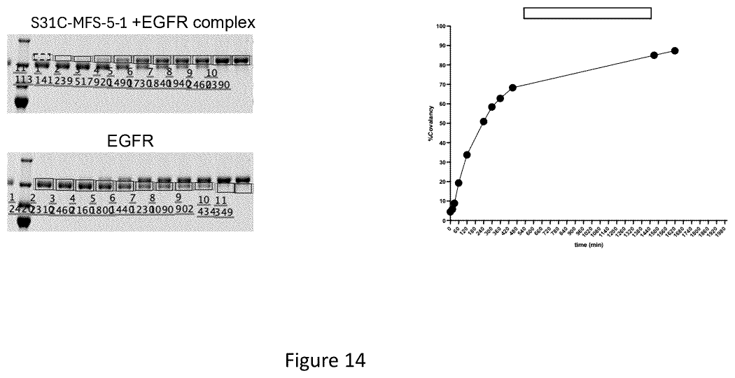

FIG. 14 is an exploded view of FIG. 13 showing either S31C-MFS-5-1:EGFR complex or EGFR alone (right) and a graph showing the percent covalency (“crosslinking”) over time. The data demonstrate that S31C-MFS-5-1 reaches 50% covalency with EGFR in 4 hours. Covalency was calculated as shown below and Table 2 summarizes the covalency data.

$$\% Covalency = \frac{T o t a l C o m p l e x}{T o t a l C o m p l e x + T o t a l E G F R}$$

TABLE 2

| | time | | | | | |
|---|---|---|---|---|---|---|
| Quant region | (min) | Signal | Total | Area | Bkgnd. | Covalency |
| Complex | 0 | 113 | 455 | 184 | 1.86 | 4.5% |
| Complex | 15 | 141 | 489 | 184 | 1.89 | 5.8% |
| Complex | 30 | 239 | 594 | 184 | 1.93 | 8.9% |
| Complex | 60 | 517 | 976 | 240 | 1.91 | 19.3% |
| Complex | 120 | 920 | 1450 | 275 | 1.92 | 33.8% |
| Complex | 240 | 1490 | 2030 | 288 | 1.9 | 50.9% |
| Complex | 300 | 1730 | 2340 | 325 | 1.89 | 58.4% |
| Complex | 360 | 1840 | 2450 | 325 | 1.89 | 62.8% |
| Complex | 450 | 1940 | 2580 | 338 | 1.89 | 68.3% |
| Complex | 1470 | 2460 | 3200 | 350 | 2.11 | 85.0% |
| Complex | 1620 | 2390 | 3260 | 364 | 2.39 | 87.3% |

| | time | | | | |
|---|---|---|---|---|---|
| Quant region | (min) | Signal | Total | Area | Bkgnd. |
| EGFR | 0 | 2420 | 3160 | 416 | 1.78 |
| EGFR | 15 | 2310 | 3050 | 400 | 1.86 |
| EGFR | 30 | 2460 | 3220 | 400 | 1.9 |
| EGFR | 60 | 2160 | 2870 | 375 | 1.88 |
| EGFR | 120 | 1800 | 2460 | 350 | 1.87 |
| EGFR | 240 | 1440 | 1980 | 288 | 1.87 |
| EGFR | 300 | 1230 | 1730 | 264 | 1.88 |
| EGFR | 360 | 1090 | 1580 | 264 | 1.88 |
| EGFR | 450 | 902 | 1360 | 240 | 1.9 |
| EGFR | 1470 | 434 | 1060 | 312 | 2 |
| EGFR | 1620 | 349 | 942 | 312 | 1.9 |

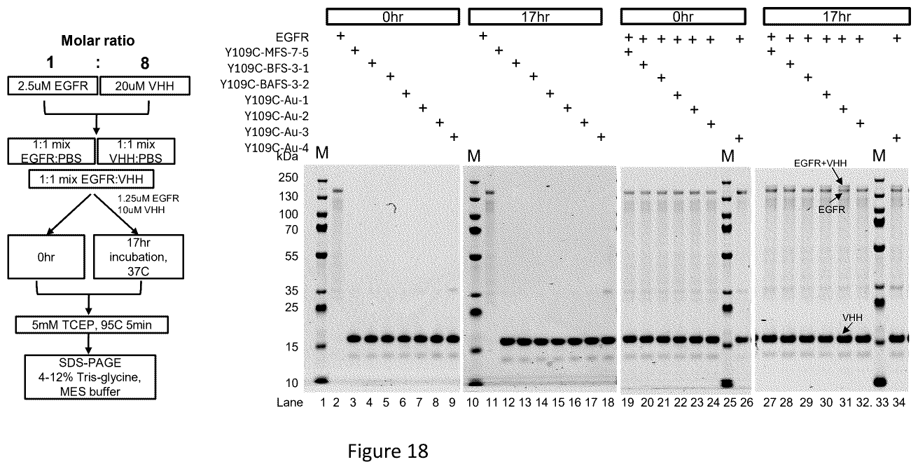

Additional binders of the present disclosure were crosslinked to their target (e.g., EGFR) and tested in gel shift assays. FIG. 18 shows a crosslinking and gel shift protocol (left) and a gel shift assay (right) after incubation between the antigen-binding domains shown and EGFR. The data demonstrate that the binders tested formed a binder:target complex. More specifically, Y109C-Au-2 exhibited the fastest crosslinking in the group tested, followed by Y109C-Au-4 and Y109C-Au-1.

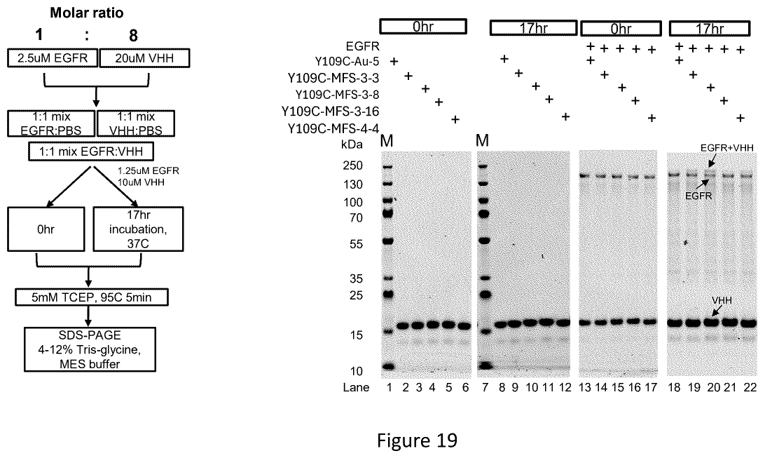

FIG. 19 shows a crosslinking and gel shift protocol (left) and a gel shift assay (right) after incubation between the antigen-binding domains shown and EGFR. The data demonstrate that the binders tested formed a binder:target complex. More specifically, Y19C-MFS-3-8 exhibited the fastest crosslinking in the group tested followed by Y119C-MFS-3-3.

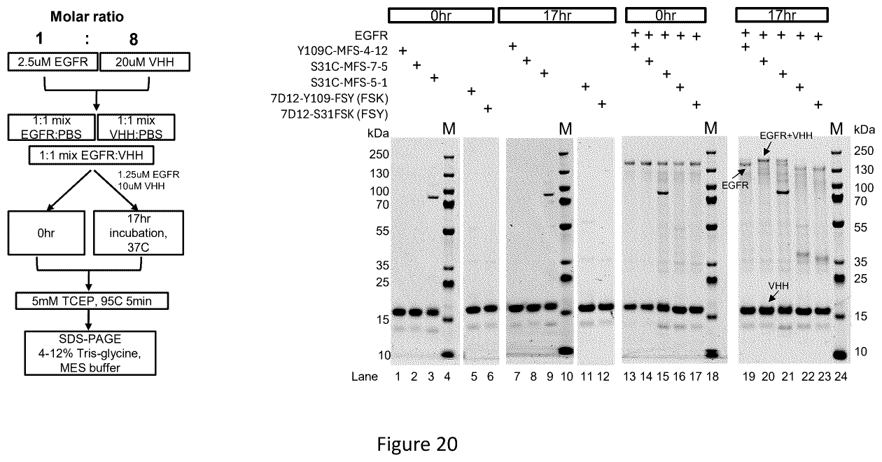

FIG. 20 shows a crosslinking and gel shift protocol (left) and a gel shift assay (right) after incubation between the antigen-binding domains shown and EGFR. The data demonstrate that S31C-MFS-7-5 exhibited 100% conversion after 17 hours of incubation.

Table 3 summarizes the percent covalency of the binders shown in FIGS. 18-20.

TABLE 3

| Test Article | Signal, complex | Signal, antigen | % Covalency |
|---|---|---|---|
| Y109C-MFS-7-5 | 31 | 166 | 16% |
| Y109C-BFS-3-1 | 34.8 | 154 | 18% |
| Y109C-BAFS-3- | 15.5 | 147 | 10% |
| 2 | | | |
| Y109C-Au-1 | 38.3 | 149 | 20% |
| Y109C-Au-2 | 60 | 132 | 31% |
| Y109C-Au-3 | 17.9 | 136 | 12% |
| Y109C-Au-4 | 46 | 146 | 24% |
| Y109C-Au-5 | 20.9 | 175 | 11% |
| Y109C-MFS-3-3 | 37.4 | 147 | 20% |
| Y109C-MFS-3-8 | 61.6 | 133 | 32% |
| Y109C-MFS-3- | 17.2 | 160 | 10% |
| 16 | | | |
| Y109C-MFS-4-4 | 31.6 | 149 | 17% |
| Y109C-MFS-4- | 25.9 | 142 | 15% |
| 12 | | | |
| S31C-MFS-7-5 | 164 | 89.3 | 65% |
| S31C-MFS-5-1 | 81.7 | 95.7 | 46% |

The data in Table 3 indicate that S31C-MFS-7-5 reaches 65% covalency after 17 hours of incubation at 37° C.; Percent covalency found: S31C-MFS-7-5>S31C-MFS-5-1>Y109C-MFS-3-8=Y109C-Au-2>remaining conjugates.

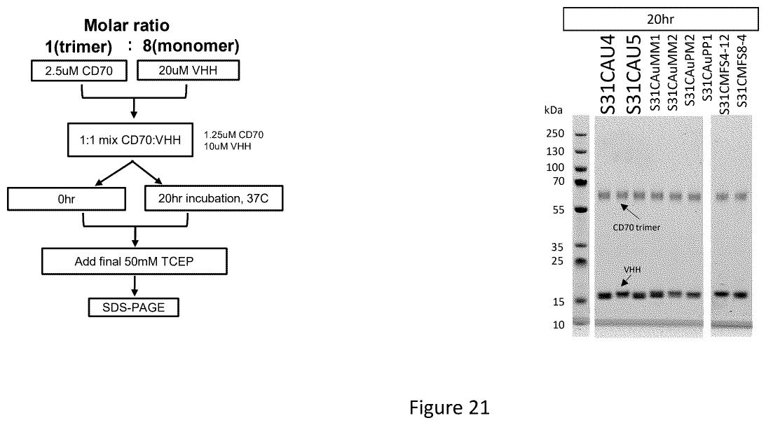

FIG. 21 shows a crosslinking and gel shift protocol (left) and a gel shift assay (right) after incubation between the antigen-binding domains shown and EGFR. The experiment was performed according to the protocol shown including incubation with CD70 protein. 7D12-S31C derived conjugates (aEGFR) (e.g., S31C-AU-4, S31C-AU-5, S31C-AU-MM2, S31C-AU-PM2, S31C-AU-PP1, S31C-MFS-4-12, and S31C-MFS-8-4) are expected to be negative to CD70 protein and as such no crosslinking observed.

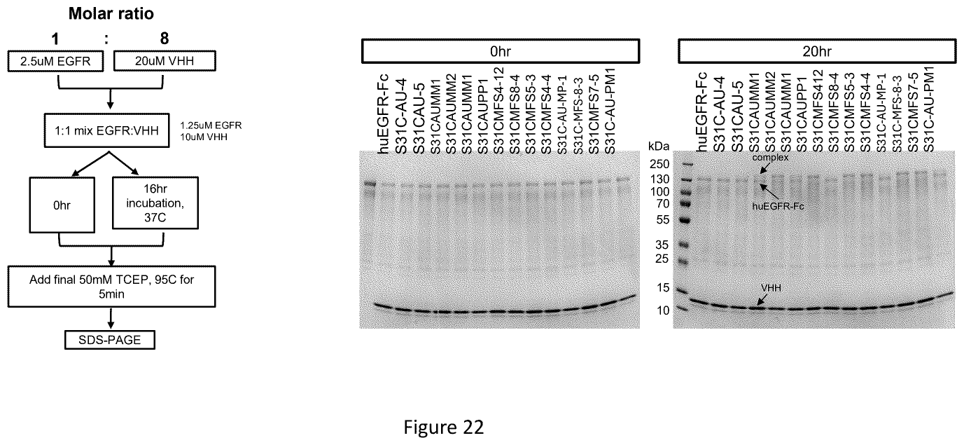

FIG. 22 shows a crosslinking and gel shift protocol (left) and a gel shift assay (right) after incubation between the antigen-binding domains shown and EGFR. The experiment was performed according to the protocol shown including incubation with EGFR protein. The data demonstrate some of the 7D12-S31C derived conjugates (e.g., S31C-AU-4, S31C-AU-5, S31C-AU-MM2, S31C-AU-PM2, S31C-AU-PP1, S31C-MFS-4-12, and S31C-MFS-8-4) formed target:binder complexes (Complex MW-144 kDa (14 kDa+130 kDa)).

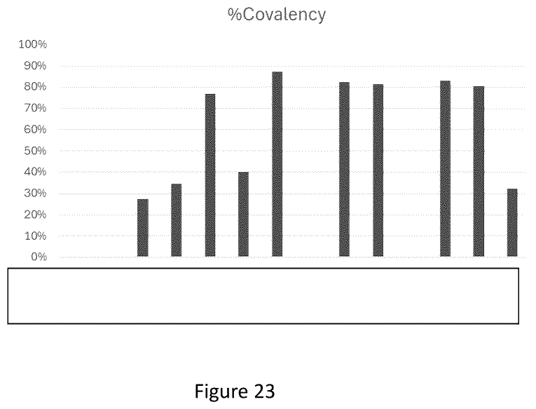

FIG. 23 is a graph showing the percent covalency of 7D12-S31C derived conjugates. Covalency was calculated as follows and the data is summarized in Table 4.

$$\% Covalency = \frac{Comp l e x}{C o m p l e x + A n t i g e n a l o n e}$$

TABLE 4

| 7D12-S31C Conjugate | Linker | % Covalency |
|---|---|---|
| S31C-AU-4 | AU-5 | 0% |
| S31C-AU-5 | AU-4 | 0% |
| S31C-MFS-4-4 | MFS-4-4 | 81% |
| S31C-MFS-4-12 | MFS-4-12 | 87% |
| S31C-MFS-5-3 | MFS-5-3 | 82% |
| S31C-AU-MP-1 | AU-MP-1 | 0% |
| S31C-AU-MM1 | AU-MM1 | 27% |
| S31C-AU-PM-1 | AU-PM-1 | 32% |
| S31C-AU-PP1 | AU-PP1 | 40% |
| S31C-AU-PM2 | AU-PM2 | 77% |
| S31C-MFS-7-5 | MFS-7-5 | 80% |
| S31C-AU-MM2 | AU-MM2 | 35% |
| S31C-MFS-8-4 | MFS-8-4 | 0% |
| S31C-MFS-8-3 | MFS-8-3 | 83% |

The data demonstrate that 7D12-S31C derivative S31C-MFS-4-12 was the most reactive binder.

FIG. 25 shows a crosslinking and gel shift protocol (left) and a gel shift assay (right) after incubation between the antigen-binding domains shown and EGFR. The experiments were performed according to the protocol shown including a 16 hour incubation period between the binder and target (EGFR). The data demonstrate complex formation (MW˜144 kDa (14 kDa+130 kDa)) with binders S31C-MFS-5-land S31C—Au-MP-6 showing the highest percentage of crosslinking.

FIG. 26 shows a crosslinking and gel shift protocol (left) and a gel shift assay (right) after incubation between the antigen-binding domains shown and EGFR. More specifically, FIG. 26 shows an exploded view of the complex formation shown in FIG. 25.

FIG. 27 shows a graph of the percent covalency for the antigen-binding domains shown. Covalency was calculated as described herein. Table 5 summarizes the covalency data for the binders shown.

TABLE 5

| Antigen-Binding Domain | % Covalency |
|---|---|
| Y109C-SFY-Au-2-1 | 13% |
| Y109C-SFY-Au-3-1 | 17% |
| Y109C-SFY-4-1 | 39% |
| Y109C-SFY-Au-4-2 | 23% |
| Y109C-SFY-Au-7-1 | 37% |
| Y109C-SFY-Au-8-1 | 30% |
| Y109C-SFY-Au-8-2 | 15% |
| Y109C-MFS-7-5 | 21% |
| Y109C-BFS-3-1 | 34% |
| Y109C-BAFS-3-2 | 18% |
| Y109C-Au-1 | 22% |
| Y109C-Au-2 | 29% |
| Y109C-Au-3 | 17% |
| Y109C-Au-4 | 22% |
| Y109C-Au-5 | 11% |
| Y109C-MFS-3-3 | 19% |
| Y109C-MFS-3-8 | 28% |
| Y109C-MFS-3-16 | 12% |
| Y109C-MFS-4-12 | 20% |
| S31C-MFS-8-4 | 16% |
| S31C-MFS-5-1 | 100% |
| S31C-MFS-8-7 | 18% |
| S31C-Au-PM-5 | 32% |
| S31C-Au-MP-6 | 100% |
| S31C-Au-PP-8 | 53% |
| S31C-Au-PM-4 | 29% |
| S31C-Au-MM-5 | 54% |
| S31C-Au-PM-6 | 57% |
| S31C-Au-MP-3 | 17% |
| S31C-Au-MP-7 | 50% |
| S31C-Au-PP-4 | 53% |
| S31C-Au-PP-5 | 50% |
| S31C-Au-PP-9 | 19% |
| S31C-SFY-Au-7-1 | 0% |
| S31C-SFY-Au-8-1 | 0% |
| S31C-SFY-Au-8-2 | 18% |
| S31C-Au-MP-5 | 52% |
| S31C-Au-MP-8 | 33% |
| S31C-Au-MM-6 | 43% |
| S31C-Au-PM-7 | 33% |
| S31C-Au-PM-8 | 42% |

The data demonstrate binders S31C-MFS-5-land S31C—Au—PP-8 exhibited the fastest crosslinking in this experiment.

### Example 154. In Vitro and Cell Based Western Assays Demonstrating Covalency Between Antigen-Binding Domains and a Binding Target

In addition to the in vitro gel shift assays performed in Example 153, Western protein blots were performed to further demonstrate covalency (i.e., conjugation) between the target (e.g., EGFR) and binders (e.g., antigen-binding domains).

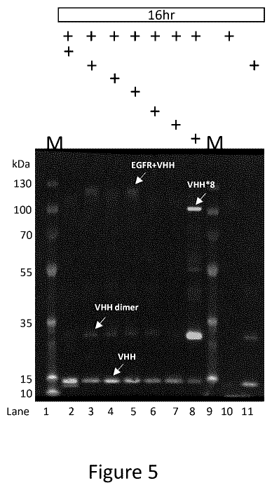

FIG. 5 shows Western protein blots that were performed according to the protocol shown in FIG. 2. The protein antibodies were transferred to a membrane and stained with a fluorescent antibody (aVHH-Alexa Fluor 790 (Catalog #128-655-230, Jackson Immuno Labs)).
Covalency was calculated as follows:

### Calculation of Covalency

1. Measure the intensity of VHH (binder), VHH dimer (binder dimer) and EGFR-VHH (target:binder) bands.
2. Designate the averaged signal of VHH-EGFR in controls a CD70-VHH and Y109C-NEMas background.
3. Subtract averaged background signal from all signals of Fluorescent signal (MFI) to Correction.
4. Calculate total values from VHH only corrected, VHH dimer corrected, and VHH-EGFR corrected.
5. Divide sum-corrected value by 8 into 1 molar.
6. Calculate the percentage between VHH-EGFR and sum as Covalency (%).

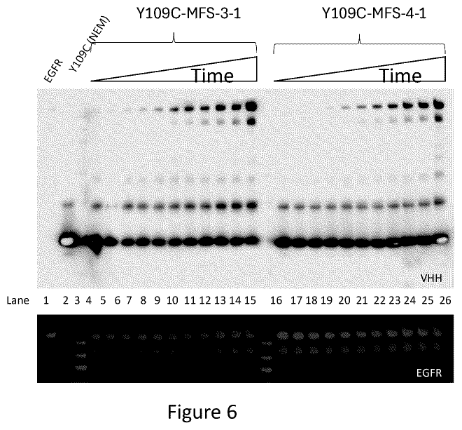

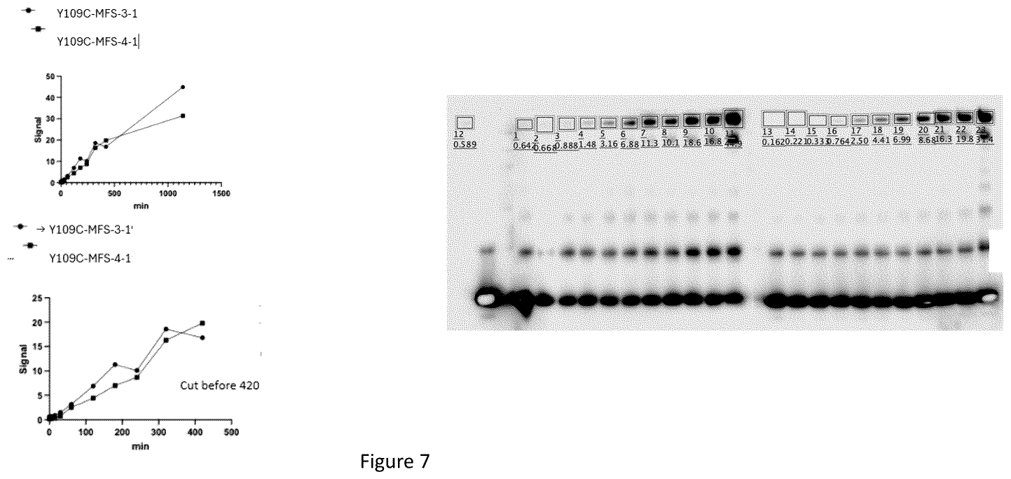

The data shown in FIG. 6 demonstrate near complete covalency (i.e., conjugation) of both Y109C-MFS-3-1 and Y109C-MFS-4-1 (7D12 Y109C derived binders) after overnight incubation and the data shown in FIG. 6 demonstrates that Y109C-MFS-3-land Y109C-MFS-4-1 form covalency over time (tested at 0, 5, 15, 30, 60, 120, 180, 240, 320, 420, and 1140 minutes) with human EGFR. FIG. 7 shows crosslinking is active as far out as 19 hours post incubation. The data also show that crosslinking increases linearly over time with a plateau observed around 5 to 7 hours post incubation.

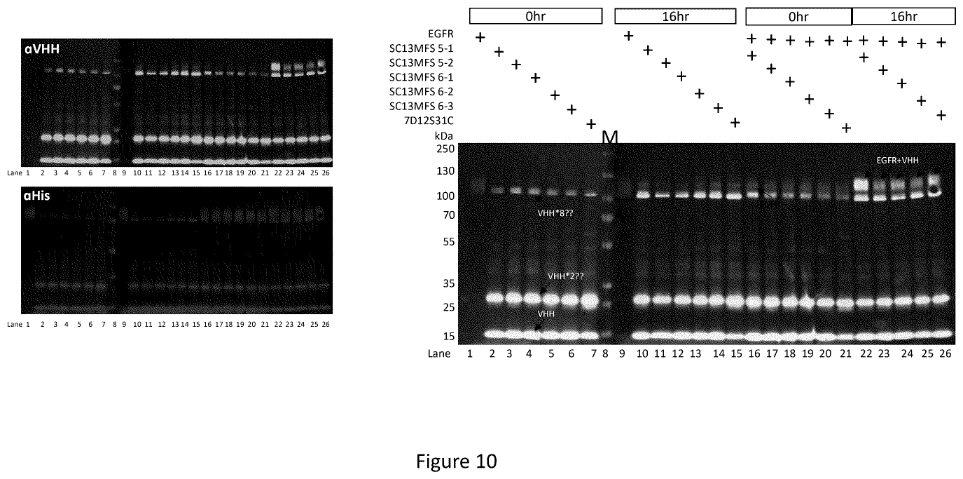

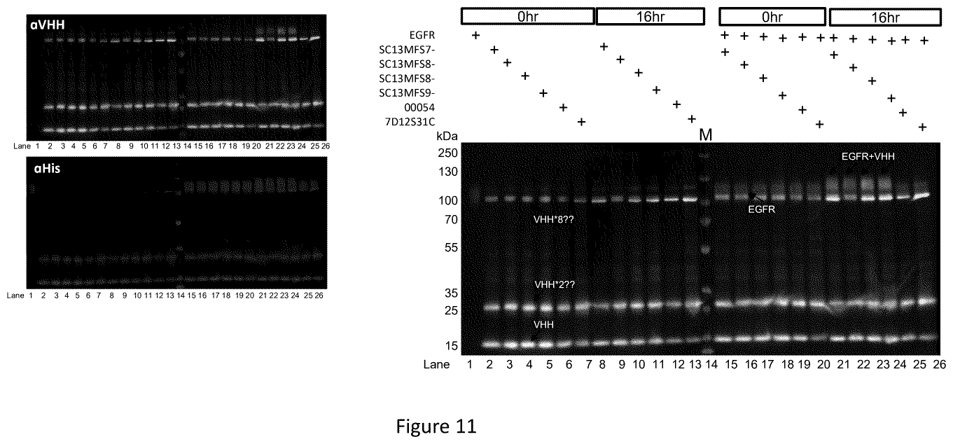

The data shown in the Western blots of FIG. 10 demonstrate that the 7D12 S31C derived binders all exhibit some degree of crosslinking with EGFR after overnight incubation. This data is consistent with the gel shift data shown in FIG. 8. Similarly, the data shown in the Western blots of FIG. 11 also exhibit some degree of crosslinking with EGFR after overnight incubation. This data is consistent with the gel shift data shown in FIG. 9.

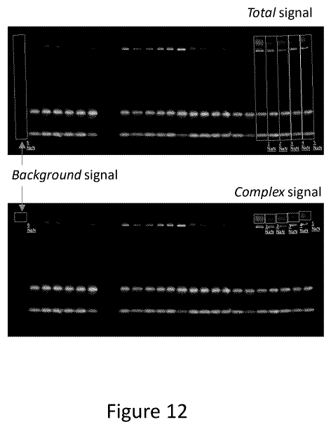

FIG. 12 are Western blots showing crosslinking between the antigen-binding domains and EGFR and generated according to the protocol shown in FIG. 2. The binders tested from were: S31C-MFS-5-1, S31C-MFS-5-2, S31C-MFS-6-1, S31C-MFS-6-2, S31C-MFS-6-3, S31C-MFS-7-4, S31C-MFS-8-2, S31C-MFS-8-1, and S31C-MFS-9-1. Control binders included 7D12 S31C-NEM and 7D12 S31C.
Covalency was calculated as follows:

### Calculation of Covalency

1. Box out the entire lane to count as Total signal.
2. Box out the complex band as Complex signal.
3. Subtract Background signal from Total and Complex.
4. Divide Total by 8.
5. Calculate % Covalency

$$\% Covalency = \frac{C o m p l e x S i g n a l - C o m p l e x B a c k g r o u n d}{T o t a l S i g n a l - T o t a l B a c k g r o u n d}$$

Table 6 below summarizes the covalency data shown in FIG. 12.

TABLE 6

| Test Article | Total | % Covalency |
|---|---|---|
| S31C-MFS-5-1 | 4.36 | 201.6% |
| S31C-MFS-5-2 | 1.85 | 63.5% |
| S31C-MFS-6-1 | 2.45 | 104.5% |
| S31C-MFS-6-2 | 1.85 | 77.4% |
| S31C-MFS-6-3 | 2.49 | 113.8% |
| S31C-MFS-7-4 | 2.71 | 87.4% |
| S31C-MFS-8-2 | 2.43 | 84.7% |
| S31C-MFS-8-1 | 3.47 | 110.4% |
| S31C-MFS-9-1 | 3.24 | 102.4% |
| 7D12 S31C-NEM | 1.6 | 34.3% |
| 7D12 S31C | 1.88 | 35.2% |

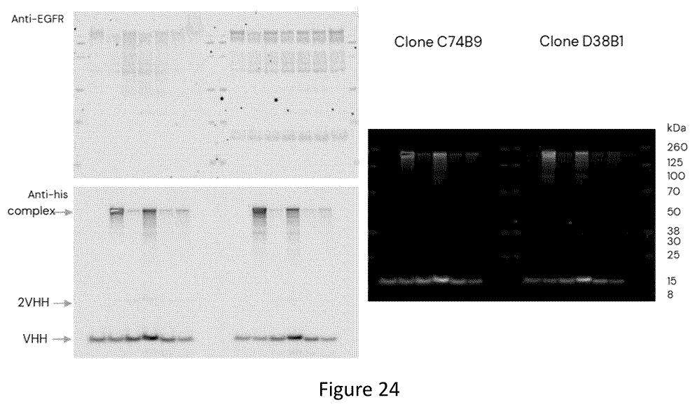

Further, FIG. 24 are Western blots showing crosslinking between the antigen-binding domains shown and EGFR. More specifically, S31C-MFS-5-1 shows modification of EGFR at 8 hours while noncovalent parent binder 7D12-S31C shows no modification.

### Cell-Based Western Assays

In order to further validate complex formation between the binders (“antigen-binding domains”) of the present disclosure and a target (e.g., EGFR), cell-based Western protein blot assays were performed. The experiments described below were performed in either EGFR+ or EGFR− expressing cell lines.

Briefly, KYSE30 cells (EGFR+expressing cells) (2×10⁵) were seeded in 12-well plates and cultured with RPMI 1640+HAM F12+10% FBS. After 24 hours, binders (either VHH (NEM capped) or VHH-FS) were added into culture for a final concentration of 1 μM in 0.5 mL volume. After a 24 hour incubation, the cells are washed twice with PBS and lysed with 100 μL RIPA buffer including a protease inhibitor cocktail for 1 hour on ice. The cell lysates were analyzed with Western blots using antibodies specific for Hisx6 (xx, 1:1000 dilution), EGFR (xx 1:1000 dilution), or GAPDH (xx, 1:1000 dilution). The Western blots detecting EGFR require a secondary anti-Rabbit incubation (xx, 1:5000 dilution). The gel images are acquired on the Licor Odessey system.

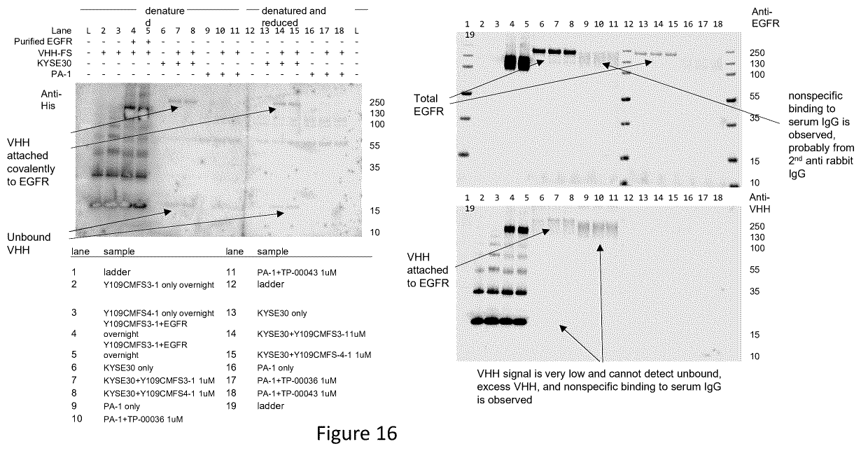

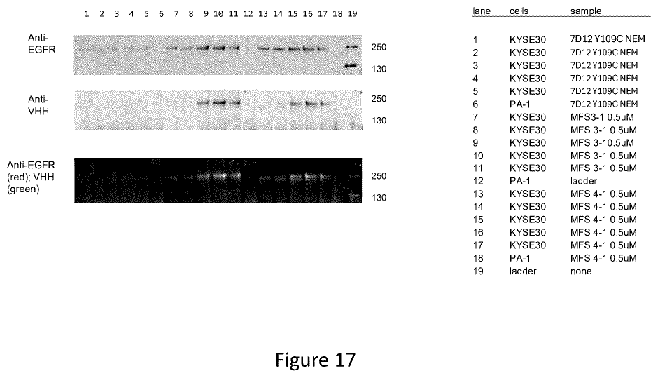

FIG. 16 are Western blots showing covalent conjugation between the antigen-binding domains shown and EGFR in either EGFR+ or EGR− cell lines. More specifically, FIG. 16 shows that EGFR:VHH-FS shows covalent attachment in EGFR positive KYSE30 cells, but not to EGFR− PA-1 cells. Similarly, FIG. 16 shows EGFR:VHH-FS covalent attachment in EGFR+KYSE30 cells starting at 4 hours.

FIG. 28 is a Western protein blot showing covalency after 24 hours between the antigen-binding domains shown and EGFR. The experiments were performed according to the cell-based assay in KYSE30 cells as described above. The data demonstrate binder:target complex formation and Table 7 summarizes the data shown in FIG. 28.

TABLE 7

| | Antigen | | | |
|---|---|---|---|---|
| | Binding | Complex | VHH only | % |
| Lane | Domain | Signal | signal | Covalency |
| 2 | Y109C-SFY- | 10.4 | 20.4 | 33.8 |
| | Au-2-1 | | | |
| 3 | Y109C-SFY- | 23.7 | 19.1 | 55.4 |
| | Au-3-1 | | | |
| 4 | Y109C-SFY-4- | 42 | 14.6 | 74.2 |
| | 1 | | | |
| 5 | Y109C-SFY- | 17.9 | 13 | 57.9 |
| | Au-4-2 | | | |
| 6 | Y109C-SFY- | 40.6 | 6.91 | 85.5 |
| | Au-7-1 | | | |
| 7 | Y109C-SFY- | 22.3 | 10.8 | 67.4 |
| | Au-8-1 | | | |
| 8 | Y109C-SFY- | 13.8 | 22.2 | 38.3 |
| | Au-8-2 | | | |
| 9 | Y109C-MFS-7- | 25.6 | 19.2 | 57.1 |
| | 5 | | | |
| 10 | Y109C-BFS-3- | 46.3 | 12.5 | 78.7 |
| | 1 | | | |
| 12 | Y109C-BAFS- | 11.2 | 23.2 | 32.6 |
| | 3-2 | | | |
| 13 | Y109C-Au-1 | 24.7 | 19.2 | 56.3 |
| 14 | Y109C-Au-2 | 51.3 | 3.72 | 93.2 |
| 15 | Y109C-Au-3 | 10.9 | 11.8 | 48 |
| 16 | Y109C-Au-4 | 35.7 | 7.7 | 82.3 |
| 17 | Y109C-Au-5 | 8.38 | 20.3 | 29.2 |
| 18 | Y109C-MFS-3- | 43.3 | 10.5 | 80.5 |
| | 3 | | | |
| 19 | Y109C-MFS-3- | 32.6 | 14.6 | 69.1 |
| | 8 | | | |
| 20 | Y109C-MFS-3- | 3.92 | 7.32 | 34.9 |
| | 16 | | | |
| 22 | Y109C-MFS-4- | 13.4 | 18 | 42.7 |
| | 12 | | | |
| 23 | S31C-MFS-8-4 | 6.26 | 14.3 | 30.4 |
| 24 | S31C-MFS-5-1 | 41.4 | 4.25 | 90.7 |
| 25 | S31C-MFS-8-7 | 9.08 | 10.2 | 47.1 |
| 26 | S31C-Au-PM-5 | 27 | 21.6 | 55.6 |
| 27 | S31C-Au-MP-6 | 49.1 | 7.45 | 86.8 |
| 28 | S31C-Au-PP-8 | 28.7 | 0.257 | 99.1 |
| 29 | S31C-Au-PM-4 | 37.8 | 36.9 | 50.6 |
| 30 | S31C-Au-MM- | 18.1 | 9.87 | 64.7 |
| | 5 | | | |
| 32 | S31C-Au-PM-6 | 37.4 | 7.46 | 83.4 |
| 33 | S31C-Au-MP-3 | 16 | 17.8 | 47.3 |
| 34 | S31C-Au-MP-7 | 43.9 | 8.46 | 83.8 |
| 35 | S31C-Au-PP-4 | 38.3 | 1.62 | 95.9 |
| 36 | S31C-Au-PP-5 | 40.3 | 4.39 | 90.2 |
| 37 | S31C-Au-PP-9 | 41.7 | 0.792 | 98.1 |
| 38 | S31C-SFY-Au- | 7.2 | 20.5 | 26 |
| | 7-1 | | | |
| 39 | S31C-SFY-Au- | 0.0391 | 20.5 | 0.2 |
| | 8-1 | | | |
| 40 | S31C-SFY-Au- | 7.44 | 18.9 | 28.2 |
| | 8-2 | | | |
| 41 | S31C-Au-MP-5 | 25.6 | 1.03 | 96.1 |
| 42 | S31C-Au-MP-8 | 38.3 | 23.1 | 62.4 |
| 43 | S31C-Au-MM- | 58 | 14.9 | 79.6 |
| | 6 | | | |
| 44 | S31C-Au-PM-7 | 59.9 | 18.6 | 76.3 |
| 45 | S31C-Au-PM-8 | 63.1 | 5.63 | 91.8 |
| 46 | none | 0.981 | 0.175 | 84.9 |

FIGS. 32 and 33 are Western blots showing rapid and robust cross-linking between the the target antigen (e.g., EGFR) and the antigen-binding domains shown in endogenously expressing EGFR cell lines. In FIGS. 33 and 34 50 nM antigen-binding domain concentration is comparable to putative human therapeutic Cₘₐₓ human; FIGS. 33 and 34 show the results in human female plasma which is also representative of male plasma results. Further, FIG. 34 shows minimal off-target covalency in plasma for the antigen-binding domains shown. Table 8 below shows in vitro crosslinking and cellular crosslinking (both at 4 hours) for the antigen-binding domains shown, including those in FIGS. 32-34.

TABLE 8

| | In vitro | | |
|---|---|---|---|
| Antigen- | protein | Cellular | |
| Binding | crosslinking | crosslinking | Plasma |
| Domain | (4 hr) | (4 hr) | Reactivity |
| Y109C- | 27% | 17% | N/A |
| SFY-Au- | | | |
| 7-1 | | | |
| S31C- | 64% | 38% | Minimal |
| MFS-5-1 | | | |
| S31C-Au- | 49% | 39% | Minimal |
| MP-6 | | | |
| S31C-Au- | 67% | 85% | Minimal |
| PP-8 | | | |
| S31C-Au- | 47% | 34% | N/A |
| PM-6 | | | |
| S31C-Au- | 53% | 63% | N/A |
| MP-5 | | | |
| S31C-Au- | 50% | 3% | N/A |
| MM-6 | | | |

Table 9 provides a summary of the experimental data included in Examples 153 and 154 above.

TABLE 9

| | Linker | | | % Covalency hu- | KYSE30 Cell |
|---|---|---|---|---|---|
| ID | Conj. Site | Description | Linker | EGFR-Fc, 16 h | @24 h % Cov |
| ″7D12 wt | native | 7D12 - native | native | | |
| ″ | | sequence | | | |
| 7D12 | S31C | 7D12 - S31C | unpaired | | |
| S31C | | | Cys | | |
| S31C- | S31C | S31C-MFS-4-4 | MFS-4-4 | 81% | |
| MFS-4-4 | | | | | |
| S31C- | S31C | S31C-MFS-4-12 | MFS-4-12 | 87% | |
| MFS-4-12 | | | | | |
| S31C- | S31C | S31C-MFS-5-1 | MFS-5-1 | 69% | 91 |
| MFS-5-1 | | | | | |
| S31C- | S31C | S31C-MFS-5-2 | MFS-5-2 | 64% | |
| MFS-5-2 | | | | | |
| S31C- | S31C | S31C-MFS-5-3 | MFS-5-3 | 82% | |
| MFS-5-3 | | | | | |
| S31C- | S31C | S31C-MFS-6-1 | MFS-6-1 | 105% | |
| MFS-6-1 | | | | | |
| S31C- | S31C | S31C-MFS-6-2 | MFS-6-2 | 77% | |
| MFS-6-2 | | | | | |
| S31C- | S31C | S31C-MFS-6-3 | MFS-6-3 | 114% | |
| MFS-6-3 | | | | | |
| S31C- | S31C | S31C-MFS-7-4 | MFS-7-4 | 87% | |
| MFS-7-4 | | | | | |
| S31C- | S31C | S31C-MFS-7-5 | MFS-7-5 | 65%/80% | |
| MFS-7-5 | | | | | |
| S31C- | S31C | S31C-MFS-8-1 | MFS-8-1 | 110% | |
| MFS-8-1 | | | | | |
| S31C- | S31C | S31C-MFS-8-2 | MFS-8-2 | 85% | |
| MFS-8-2 | | | | | |
| S31C- | S31C | S31C-MFS-8-3 | MFS-8-3 | 83% | |
| MFS-8-3 | | | | | |
| S31C- | S31C | S31C-MFS-8-4 | MFS-8-4 | 16% | 30 |
| MFS-8-4 | | | | | |
| S31C- | S31C | S31C-MFS-8-7 | MFS-8-7 | 19% | 47 |
| MFS-8-7 | | | | | |
| S31C- | S31C | S31C-MFS-9-1 | MFS-9-1 | 102% | |
| MFS-9-1 | | | | | |
| S31C-AU- | S31C | S31C-AU-4 | Au-4 | 0% | |
| 4 | | | | | |
| S31C-AU- | S31C | S31C-AU-5 | Au-5 | 0% | |
| 5 | | | | | |
| S31C-AU- | S31C | S31C-AU-MM-1 | Au-MM-1 | 27% | |
| MM1 | | | | | |
| S31C-AU- | S31C | S31C-AU-MM-2 | Au-MM-2 | 35% | |
| MM2 | | | | | |
| S31C-AU- | S31C | S31C-AU-MP-1 | Au-MP-1 | 0% | |
| MP-1 | | | | | |
| S31C-AU- | S31C | S31C-AU-PM-1 | Au-PM-1 | 32% | |
| PM-1 | | | | | |
| S31C-AU- | S31C | S31C-AU-PM-2 | Au-PM-2 | 77% | |
| PM2 | | | | | |
| S31C-AU- | S31C | S31C-AU-PP-1 | Au-PP-1 | 40% | |
| PP1 | | | | | |
| S31C-Au- | S31C | S31C-Au-PM-5 | Au-PM-5 | 32% | 56 |
| PM-5 | | | | | |
| S31C-Au- | S31C | S31C-Au-MP-6 | Au-MP-6 | 68% | 87 |
| MP-6 | | | | | |
| S31C-Au- | S31C | S31C-Au-PP-8 | Au-PP-8 | 60% | 99 |
| PP-8 | | | | | |
| S31C-Au- | S31C | S31C-Au-PM-4 | Au-PM-4 | 29% | 51 |
| PM-4 | | | | | |
| S31C-Au- | S31C | S31C-Au-MM-5 | Au-MM-5 | 63% | 65 |
| MM-5 | | | | | |
| S31C-Au- | S31C | S31C-Au-PM-6 | Au-PM-6 | 64% | 83 |
| PM-6 | | | | | |
| S31C-Au- | S31C | S31C-Au-MP-3 | Au-MP-3 | 17% | 47 |
| MP-3 | | | | | |
| S31C-Au- | S31C | S31C-Au-MP-7 | Au-MP-7 | 52% | 84 |
| MP-7 | | | | | |
| S31C-Au- | S31C | S31C-Au-PP-4 | Au-PP-4 | 55% | 96 |
| PP-4 | | | | | |
| S31C-Au- | S31C | S31C-Au-PP-5 | Au-PP-5 | 50% | 90 |
| PP-5 | | | | | |
| S31C-Au- | S31C | S31C-Au-PP-9 | Au-PP-9 | 20% | 98 |
| PP-9 | | | | | |
| S31C-Au- | S31C | S31C-Au-MP-5 | Au-MP-5 | 52% | 96 |
| MP-5 | | | | | |
| S31C-Au- | S31C | S31C-Au-MP-8 | Au-MP-8 | 34% | 62 |
| MP-8 | | | | | |
| S31C-Au- | S31C | S31C-Au-MM-6 | Au-MM-6 | 43% | 80 |
| MM-6 | | | | | |
| S31C-Au- | S31C | S31C-Au-PM-7 | Au-PM-7 | 34% | 76 |
| PM-7 | | | | | |
| S31C-Au- | S31C | S31C-Au-PM-8 | Au-PM-8 | 43% | 92 |
| PM-8 | | | | | |
| S31C- | S31C | S31C-SFY-Au-7- | SFY-Au- | 0% | 26 |
| SFY-Au- | | 1 | 7-1 | | |
| 7-1 | | | | | |
| S31C- | S31C | S31C-SFY-Au-8- | SFY-Au- | 0% | 0 |
| SFY-Au- | | 1 | 8-1 | | |
| 8-1 | | | | | |
| S31C- | S31C | S31C-SFY-Au-8- | SFY-Au- | 19% | 28 |
| SFY-Au- | | 2 | 8-2 | | |
| 8-2 | | | | | |
| 7D12 | Y109C | 7D12 - Y109C | unpaired | | |
| Y109C | | | Cys | | |
| Y109C- | Y109C | 7D12 - Y109C - | NEM | 0 | |
| NEM | | NEM | | | |
| Y109C- | Y109C | Y109C-MFS-3-1 | MFS-3-1 | 133% | |
| MFS-3-1 | | | | | |
| Y109C- | Y109C | Y109C-MFS-3-2 | MFS-3-2 | 21% | |
| MFS-3-2 | | | | | |
| Y109C- | Y109C | Y109C-MFS-3-3 | MFS-3-3 | 19% | 81 |
| MFS-3-3 | | | | | |
| Y109C- | Y109C | Y109C-MFS-3-8 | MFS-3-8 | 29% | 69 |
| MFS-3-8 | | | | | |
| Y109C- | Y109C | Y109C-MFS-3- | MFS-3-16 | 10% | 35 |
| MFS-3-16 | | 16 | | | |
| Y109C- | Y109C | Y109C-MFS-4-1 | MFS-4-1 | 115% | |
| MFS-4-1 | | | | | |
| Y109C- | Y109C | Y109C-MFS-4-4 | MFS-4-4 | 17% | |
| MFS-4-4 | | | | | |
| Y109C- | Y109C | Y109C-MFS-4- | MFS-4-12 | 20% | 43 |
| MFS-4-12 | | 12 | | | |
| Y109C- | Y109C | Y109C-MFS-7-5 | MFS-7-5 | 19% | 57 |
| MFS-7-5 | | | | | |
| Y109C- | Y109C | Y109C-BFS-3-1 | BFS-3-1 | 30% | 79 |
| BFS-3-1 | | | | | |
| Y109C- | Y109C | Y109C-BAFS-3- | BAFS-3-2 | | |
| BAFS-3-2 | | 2 | | | |
| Y109C- | Y109C | Y109C-Au-1 | Au-1 | 21% | 56 |
| Au-1 | | | | | |
| Y109C- | Y109C | Y109C-Au-2 | Au-2 | 30% | 93 |
| Au-2 | | | | | |
| Y109C- | Y109C | Y109C-Au-3 | Au-3 | 15% | 48 |
| Au-3 | | | | | |
| Y109C- | Y109C | Y109C-Au-4 | Au-4 | 22% | 82 |
| Au-4 | | | | | |
| Y109C- | Y109C | Y109C-Au-5 | Au-5 | 10% | 29 |
| Au-5 | | | | | |
| Y109C- | Y109C | Y109C-FP-FS-1 | FP-FS-1 | 0 | |
| FP-FS-1 | | | | | |
| Y109C- | Y109C | Y109C-SFY-Au- | SFY-Au- | 12% | 34 |
| SFY-Au- | | 2-1 | 2-1 | | |
| 2-1 | | | | | |
| Y109C- | Y109C | Y109C-SFY-Au- | SFY-Au- | 13% | 55 |
| SFY-Au- | | 3-1 | 3-1 | | |
| 3-1 | | | | | |
| Y109C- | Y109C | Y109C-SFY-4-1 | SFY-4-1 | 39% | 74 |
| SFY-4-1 | | | | | |
| Y109C- | Y109C | Y109C-SFY-Au- | SFY-Au- | 15% | 58 |
| SFY-Au- | | 4-2 | 4-2 | | |
| 4-2 | | | | | |
| Y109C- | Y109C | Y109C-SFY-Au- | SFY-Au- | 37% | 86 |
| SFY-Au- | | 7-1 | 7-1 | | |
| 7-1 | | | | | |
| Y109C- | Y109C | Y109C-SFY-Au- | SFY-Au- | 28% | 67 |
| SFY-Au- | | 8-1 | 8-1 | | |
| 8-1 | | | | | |
| Y109C- | Y109C | Y109C-SFY-Au- | SFY-Au- | 14% | 38 |
| SFY-Au- | | 8-2 | 8-2 | | |
| 8-2 | | | | | |

Further, table 10 shows the intact mass of exemplary antigen-binding domains and the associated linkers shown.

TABLE 10

| VHH | Linker | Linker Structure | Intact Mass (DAR1) |
|---|---|---|---|
| 7D12- Y109C | SFY-Au-2-1 | 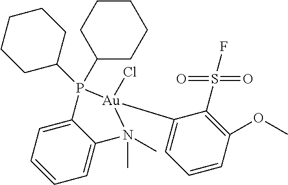 | 14352.8 |
| | SFY-Au-3-1 | 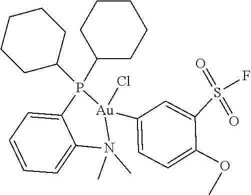 | 14369.9 |
| | SFY-4-1 | 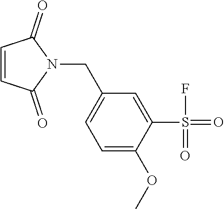 [SMILES: COc1ccc(CN2C(=O)C=CC2=O)cc1S(=O)(=O)F] | 14463.9 |
| | SFY-Au-4-2 | 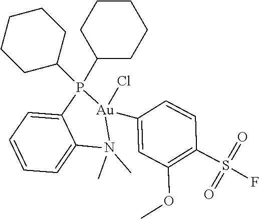 | 14353.2 |
| | SFY-Au-7-1 | 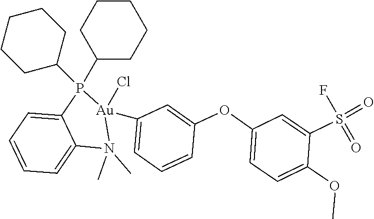 | 14444.9 |
| | SFY-Au-8-1 | 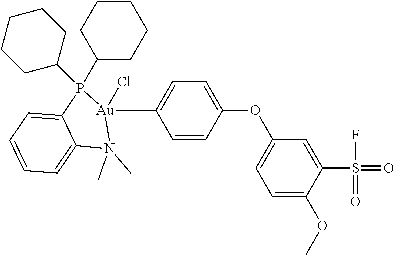 | 14445.0 |
| | SFY-Au-8-2 | 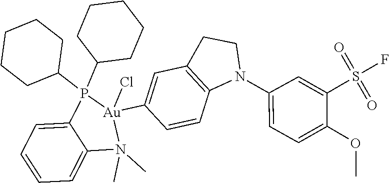 | 14469.7 |
| VHH | Linker Code | Linker Structure | Intact Mass (DAR1) |
| 7D12- S31C | MFS-8-7 | 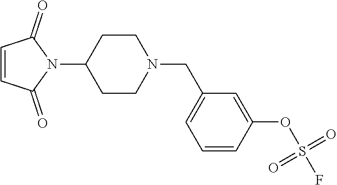 [SMILES: O=C1C=CC(=O)N1C1CCN(Cc2cccc(OS(=O)(=O)F)c2)CC1] | 14609.5 |
| | Au-PM-5 | 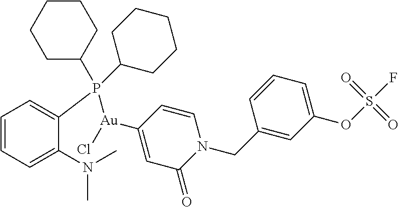 | 14522.3 |
| | Au-MP-6 | 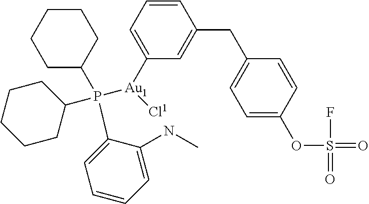 | 14505.2 |
| | Au-PP-8 | 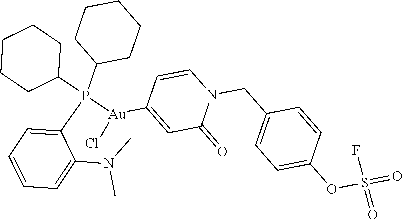 | 14522.2 |
| | Au-PM-4 | 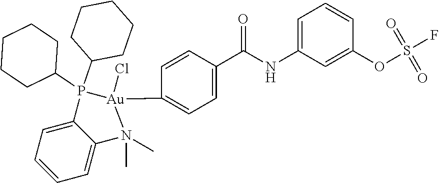 | 14534.6 |
| | Au-MM-5 | 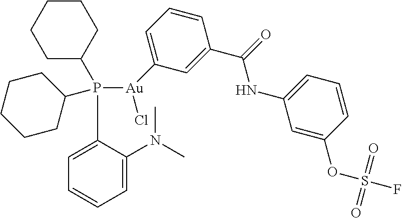 | 14534.1 |
| | Au-PM-6 | 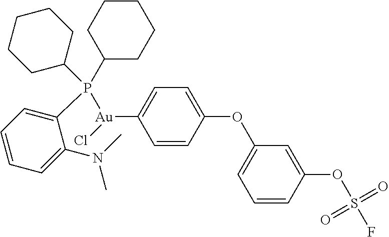 | 14507.1 |
| | Au-MP-3 | 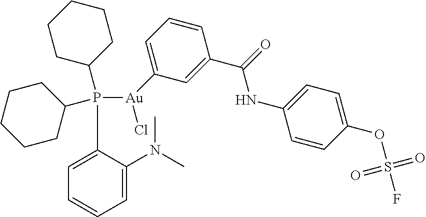 | 14534.0 |
| | Au-MP-7 | 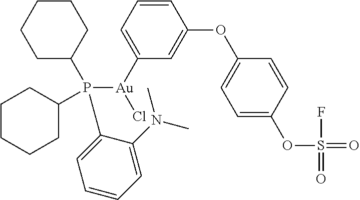 | 14506.9 |
| | Au-PP-4 | 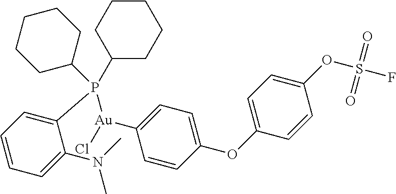 | 14506.9 |
| | Au-PP-5 | 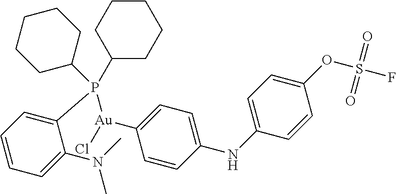 | 14506.2 |
| | Au-PP-9 | 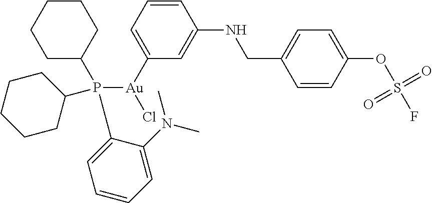 | 14520.6 |
| 7D12- S31C | Au-MP-5 | 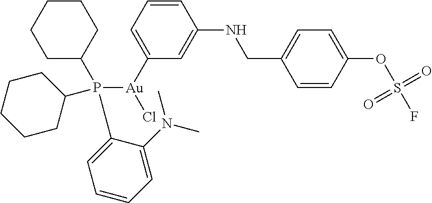 | 14520.2 |
| | Au-MP-8 | 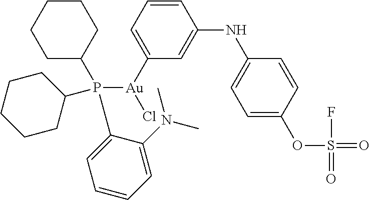 | 14506.2 |
| | Au-MM-6 | 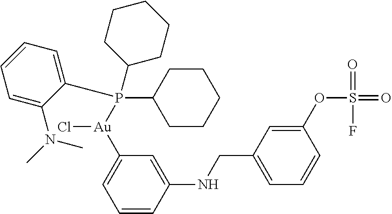 | 14520.2 |
| | Au-PM-7 | 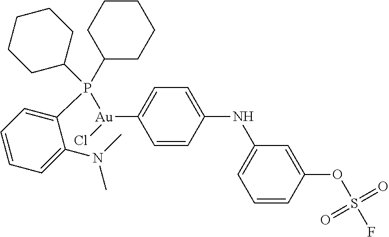 | 14506.2 |
| | Au-PM-8 | 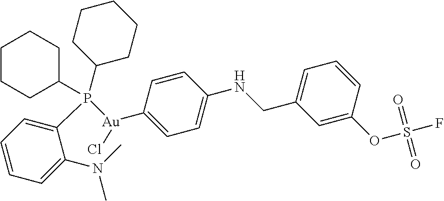 | 14520.2 |
| | SFY-Au-7-1 | 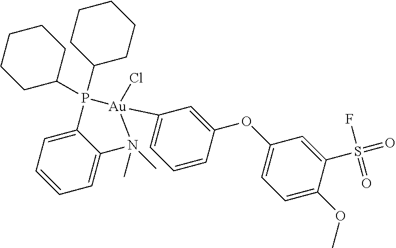 | 14519.4 |
| | SFY-Au-8-1 | 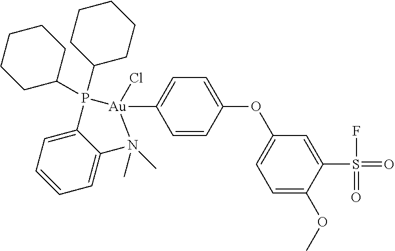 | 14519.4 |
| | SFY-Au-8-2 | 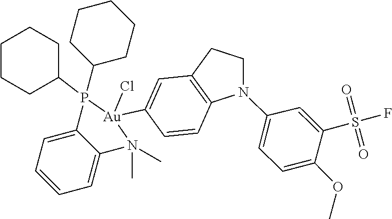 | 14546.7 |

### Example 155. Mass Spectrometry Analysis

Mass Spectrometry was performed to determine the kinetic covalency by intact mass and to confirm the site of covalency of the target by tandem mass spectrometry (MS/MS) (i.e., peptide mapping). DTM (e.g., charge detection) was used since it is the only way to discern different protein species without deglycosylation.

Briefly, after incubation between antigen-binding domain Y109C-MFS-4-1 and EGFR intact mass spectrometry was performed. Buffer was exchanged into 200 mM ammonium acetate and ran under native conditions.

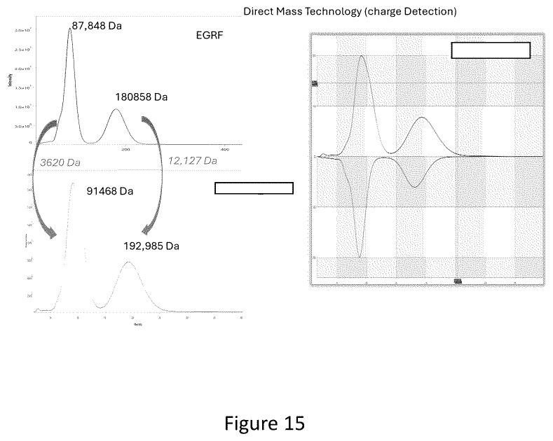

FIG. 15 demonstrates that a mass shift was observed. Although the mass shift observed did not align with the expected mass shift, the data show both qualitatively and quantitatively different species of conjugated and non-conjugated samples since both more charge was observed and the number of ions in each peak shown increased, respectively. Table 8 shows the intact mass for additional antigen-binding domain and linker conjugates.

Protein Complex Formation and Sample Preparation Purified human epidermal growth factor receptor (EGFR) extracellular domain (residues 25-645, C-terminally His-tagged; Acro Biosystems; 1430 μmol), was mixed with purified, linker-modified antigen-binding domain (SEQ ID 25) or (SEQ ID 27) (2717 μmol) in PBS, pH 7.2 in a final volume of 207 μl and incubated for 18 hours at 37° C. to allow crosslinking. For reduction and denaturation, 67.5 μl of this reaction was treated with 78 nmol tris(2-carboxyethyl)phosphine (TCEP) and incubated at 70° C. for 5 minutes. N-linked glycans were removed by adding 975 units of recombinant PNGase F (New England Biolabs) followed by incubation at 50° C. for 45 minutes. FIG. 29 shows SDS-PAGE analysis of preparative crosslinking reactions prior to digestion for LC-MS/MS. Lanes are identified by antigen-binding domain and preparation steps (X, crosslinking reaction after overnight incubation at 37° C.; P, crosslinking reaction after further treatment with heat PNGase F). Bands are labeled by chemical species as inferred from apparent molecular weight. Negligible unreacted target protein is detected in reaction samples with both components.

76 μl of the deglycosylated sample was processed for LC-MS/MS with the AccuMAP™ Low pH Protein Digestion Kit (Promega) according to the manufacturer's protocol for proteolytically resistant proteins. Briefly, the sample was denatured with guanidine hydrochloride, reduced with TCEP, alkylated with iodoacetamide, and then digested with recombinant Lys-C and modified Trypsin. Peptides were desalted with Pierce C18 Spin Columns (ThermoFisher Scientific), and acetonitrile was removed by vacuum centrifugation. The resulting peptides were resuspended with 0.1% formic acid, 2% acetonitrile in water to a final concentration of approximately 9 μg/μl, as determined by the Pierce Quantitative Fluorometric Peptide Assay Kit (ThermoFisher Scientific).

### Liquid Chromatography—Tandem Mass Spectrometry (LC-MS/MS)

For LC-MS/MS analysis, 10 μg of peptide digest was injected onto a Waters ACQUITY Premier CSH C18 column (1.7 μm, 2.1×100 mm, 130 Å pore size) using a Vanquish Horizon Flex UHPLC system with a binary pump (ThermoFisher Scientific) Peptides were eluted at a flow rate of 0.3 ml/min using a linear gradient from 2% to 30% acetonitrile in 0.1% formic acid over 120 min. The column temperature was maintained at 50° C.

Eluted peptides were analyzed on an Orbitrap Exploris 240 mass spectrometer equipped with a heated electrospray ionization (H-ESI) source (ThermoFisher Scientific). The spray voltage was 3500 V (positive mode). The probe vaporizer and ion transfer tube temperatures were 300° C. and 340° C., respectively. Full MS scans were acquired in the Orbitrap from m/z 300-1800 at a resolution of 60,000, with an RF lens setting of 70%, an automatic gain control (AGC) target of 3e6, and a maximum injection time of 80 ms. Lock mass correction with the EASY-IC internal calibrant was enabled.

Data-dependent (DDA) was performed with a fixed cycle time of 3 s, selecting precursor ions with charge states 3-8 and intensities above 5e3 for MS/MS. Dynamic exclusion was enabled with a ±10 ppm mass tolerance and a 5 s exclusion duration. Selected precursors were isolated using a 3 m/z window and fragmented by stepped higher-energy collisional dissociation (HCD) at normalized collision energies of 23%, 30%, and 37%. MS2 spectra were recorded in the Orbitrap in centroid mode with a resolution of 15,000, an AGC target of 1e5, maximum injection time of 250 ms, one microscan, and automatic mass range determination.

### Data Analysis

Peaklist files in mascot generic format (mgf) were generated from Raw files using a python interface to RawFileReader.dll (Thermo). Peaklists were initially search to identify proteins and any artifacts in the sample, followed by a search for crosslinked peptides using Protein Prospector v6.6.24 restricted to the most abundant proteins identified (ref—Trnka et al MCP 2014, https://www.mcponline.org/article/S1535-9476(20)34635-1/fulltext). The linkers were parameterized as heterobifunctional crosslinking reagents with Cys specificity on one side and nucleophile specificity on the other: His, Lys, Tyr, Ser, Thr, or Protein N-terminus. Maleimide containing linkers were search as both the canonical, ring-closed maleimide adduct and the hydrolyzed, ring-open succinamic acid adduct. Dead-end crosslinks were parameterized as the species in which the Cys-directed site has reacted and the fluorosulfonate/sulfonyl fluoride moiety remains intact. Database searches were performed against the sequences of the EGFR construct and the VHH with the appropriate Cys mutation (7D12 S31C). Enzyme specificity was Trypsin with 2-missed cleavages and precursor and product ion tolerances were set to 8 ppm and 20 ppm respectively. No constant modifications were specified. Up to 4 variable modifications per peptide were allowed from the following list: Acetyl (Protein N-term), Acetyl+Oxidation (Protein N-term), Carbamidomethylation (C), Deamidation (restricted to Asn residues in the N-glycan consensus motif: N[\hat{ }][ST]), Gln->pyroGlu (Peptide N-term), Met-loss (Protein N-term), Met-loss+Acetyl (Protein N-term), Oxidation (M), and the Cys-reacted dead-end modification. The top 100 most intense ion-signals were used for the search and the Max_Peptide Permutations parameter was set to 1000. Mass Modifications were searched through a range of 400-5000 Da and the top 1000 peptide matches to each spectrum were saved for the crosslink search. Crosslinked peptides were reported that had a Score Difference >10. Each of the annotated crosslinked spectral matches were manually examined for features such as: robust uences of product ions matched from both peptides, prevalence of prominent unmatched ion-signals or other noise, and whether the site-localization was supported by the product ion matches.

FIGS. 30 and 31 show exemplary mass spectrometry data indicating successful antigen-binding domain and linker preparation. FIGS. 30 and 31 show representative MS2 spectra for S31C-MFS-5-1 (FIG. 30) and S31C—Au-PM-5 (FIG. 31) showing evidence for crosslinking between Cys 31 of the antigen-binding domain and His 349 of EGFR. Major ion series and internal fragments are labeled, and ions consistent with site localization to His 349 of EGFR are highlighted.

FIG. 35 shows crosslink spectral matches from tandem mass spectrometry for exemplary antigen-binding domains with linkers after reacting with antigen. Spectra corresponding FIGS. 30 and 31 are highlighted.

What is claimed is:

1. A compound having formula (I): 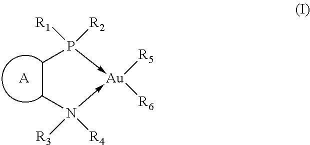
Ring A is:
C₆₋₁₄ aryl optionally substituted with 1-4 substituents independently selected from the group consisting of oxo, R^{a}, and R^{b}; or
heteroaryl of 5-12 ring atoms, wherein 1-4 ring atoms are heteroatoms, each independently selected from the group consisting of N, N(H), N(R^{c}), O, and S(O)₀₋₂, and wherein the heteroaryl is optionally substituted with 1-4 substituents independently selected from the group consisting of oxo, R^{a}, and R^{b};
each of R¹ and R² is independently selected from the group consisting of:
C₆₋₁₄ aryl optionally substituted with 1-4 substituents independently selected from the group consisting of oxo, R^{a}, and R^{b}; and
C₃₋₁₂ cycloalkyl optionally substituted with substituents independently selected from the group consisting of oxo, R^{a}, and R^{b};
each of R³ and R⁴ is independently selected from the group consisting of:
C₁₋₁₀ alkyl optionally substituted with 1-4 independently selected R^{d}; and
C₃₋₁₂ cycloalkyl optionally substituted with substituents independently selected from the group consisting of oxo, R^{a}, and R^{b};
R⁵ is a coordinating anion;
R⁶ is —*R⁶¹-R⁶²-R⁶³; wherein, the * indicates the point of attachment of R⁶¹-R⁶²-R⁶³ to Au:
R⁶¹ is:
divalent C₆₋₁₀ aryl optionally substituted with 1-4 substituents independently selected from the group consisting of oxo, R^{a}, and R^{b}; or
divalent heteroaryl of 5-10 ring atoms, wherein 1-4 ring atoms are heteroatoms, each independently selected from the group consisting of N, N(H), N(R^{c}), O, and S(O)₀₋₂, and wherein the heteroaryl is optionally substituted with 1-4 substituents independently selected from the group consisting of oxo, R^{a}, and R^{b};
R⁶² is absent or is C₁-C₁₆ alkylene, C₂-C₁₆ alkenylene, or C₂-C₁₆ alkynylene, each of which is optionally substituted with 1-2 substituents independently selected from halogen and C₃-C₆ cycloalkyl; and wherein 1-8 alkylene units are optionally replaced with a group independently selected from the group consisting of:
(i) —O—;
(ii) —NH—;
(iii) —N(C₁-C₆ alkyl)-;
(iv) —C(O)—;
(v) —S—;
(vi) —S(O)—;
(vii) —S(O)₂—;
(viii) divalent C₃-C₁₀ cycloalkyl, which is optionally substituted with 1-4 R^{a};
(ix) divalent C₆-C₁₀ aryl, which is optionally substituted with 1-4 R^{a};
(x) divalent heteroaryl of 5-10 ring atoms, wherein 1-4 ring atoms are heteroatoms, each independently selected from the group consisting of N, N(H), N(R^{c}), O, and S(O)₀₋₂; and which is optionally substituted with 1-4 R^{a}; and
(xi) divalent heterocyclyl of 3-10 ring atoms, wherein 1-3 ring atoms are heteroatoms, each independently selected from the group consisting of N, N(H), N(R^{c}), N(O⁻), O, and S(O)₀₋₂, and which is optionally substituted with 1-4 R^{a};
R⁶³ is a reactive group;
each occurrence of R^{a} is independently selected from the group consisting of: halo; cyano;
C₁₋₁₀ alkyl which is optionally substituted with 1-6 independently selected R^{d}; C₂₋₆ alkenyl; C₂₋₆ alkynyl; C₁₋₄ alkoxy; C₁₋₄ haloalkoxy; —S(O)₁₋₂(C₁₋₄ alkyl); —S (O)(═NH)(C₁₋₄ alkyl); —NR^{e}R^{f}; —OH; —S(O)₁₋₂NR′R″; —C₁₋₄ thioalkoxy; —NO₂; —C(═O)(C₁₋₁₀ alkyl); —C(═O)O(C₁₋₄ alkyl); —OC(═O)(C₁₋₄ alkyl); —C(═O)OH; —C(═O)NR′R″; —NR′C(═O)(C₁₋₄ alkyl) and —SF₅;
each occurrence of R^{b} is independently selected from the group consisting of:
L¹-C₃₋₁₂ cycloalkyl or C₃₋₁₂ cycloalkenyl, each of which is optionally substituted with 1-4 substituents independently selected from the group consisting of oxo and R^{d};
L¹-heterocyclyl or L¹-heterocycloalkenyl of 3-12 ring atoms, wherein 1-3 ring atoms are heteroatoms, each independently selected from the group consisting of N, N(H), N(R^{c}), O, and S(O)₀₋₂, and wherein the heterocyclyl or heterocycloalkenyl is optionally substituted with 1-4 substituents independently selected from the group consisting of oxo and R^{d};
L¹-heteroaryl of 5-12 ring atoms, wherein 1-4 ring atoms are heteroatoms, each independently selected from the group consisting of N, N(H), N(R^{c}), O, and S(O)₀₋₂, and wherein the heteroaryl is optionally substituted with 1-4 substituents independently selected from the group consisting of oxo and R^{d}; and
L¹-C₆₋₁₀ aryl optionally substituted with 1-4 substituents independently selected from the group consisting of oxo and R^{d};
L₁ is a bond or C₁₋₄ alkylene;
each occurrence of R^{c} is independently selected from the group consisting of: C₁₋₆ alkyl optionally substituted with 1-3 independently selected R^{a}; —C(O)(C₁₋₄ alkyl); —C(O)O(C₁₋₄ alkyl); —CONR′R″; —S(O)₁₋₂NR′R″; —S(O)₁₋₂(C₁₋₄ alkyl); —OH; and C₁₋₄ alkoxy;
each occurrence of R^{d} is independently selected from the group consisting of: —OH; -halo; —NR^{e}R^{f}, C₁₋₄ alkoxy; C₁₋₄ haloalkoxy; —C(═O)O(C₁₋₄ alkyl); —C(═O)(C₁₋₄ alkyl); —OC(═O)(C₁₋₄ alkyl); —C(═O)OH; —CONR′R″; —S(O)₁₋₂NR′R″; —S(O)₁₋₂(C₁₋₄ alkyl); and cyano;
each occurrence of Re and R^{f} is independently selected from the group consisting of: H; C₁₋₆ alkyl optionally substituted with 1-3 substituents each independently selected from the group consisting of NR′R″, —OH, halo, C₁₋₄ alkoxy, and C₁₋₄ haloalkoxy; —C(O)(C₁₋₄ alkyl); —C(O)O(C₁₋₄ alkyl); —CONR′R″; —S(O)₁₋₂NR′R″; —S(O)₁₋₂(C₁₋₄ alkyl); —OH; and C₁₋₄ alkoxy; and
each occurrence of R′ and R″ is independently selected from the group consisting of: H; —OH; and C₁₋₄ alkyl.

2. The compound of claim 1, wherein R⁶¹ is divalent C₆₋₁₀ aryl optionally substituted with 1-4 substituents independently selected from the group consisting of oxo, R^{a}, and R^{b}.

3. The compound of claim 1 or 2, wherein R⁶¹ is divalent phenyl optionally substituted with 1-4 substituents independently selected from the group consisting of R^{a} and R^{b}.

4. The compound of any one of claims 1-3, wherein R⁶¹ is divalent phenyl optionally substituted with 1-4 independently selected R^{a}.

5. The compound of any one of claims 1-4, wherein R⁶¹ is divalent phenyl optionally substituted with 1-2 independently selected R^{a}.

6. The compound of any one of claims 2-5, wherein each occurrence of R^{a} is independently selected from the group consisting of halo and C₁₋₁₀ alkyl which is optionally substituted with 1-6 independently selected R^{d}, optionally wherein each occurrence of R^{a} is independently selected from the group consisting of fluoro and CH₃.

7. The compound of claim 1, wherein R⁶¹ is divalent heteroaryl of 5-10 ring atoms, wherein 1-4 ring atoms are heteroatoms, each independently selected from the group consisting of N, N(H), N(R^{c}), O, and S(O)₀₋₂, and wherein the heteroaryl is optionally substituted with 1-4 substituents independently selected from the group consisting of oxo, R^{a}, and R^{b}.

8. The compound of any one of claims 1-7, wherein R⁶² is absent.

9. The compound of any one of claims 1-7, wherein R⁶² is C₁-C₁₆ alkylene, C₂-C₁₆ alkenylene, or C₂-C₁₆ alkynylene, each of which is optionally substituted with 1-2 substituents independently selected from halogen and C₃-C₆ cycloalkyl; and wherein 1-8 alkylene units are optionally replaced with a group independently selected from the group consisting of:
(i) —O—;
(ii) —NH—;
(iii) —N(C₁-C₆ alkyl)-;
(iv) —C(O)—;
(v) —S—;
(vi) —S(O)—;
(vii) —S(O)₂—;
(viii) divalent C₃-C₁₀ cycloalkyl, which is optionally substituted with 1-4 R^{a};
(ix) divalent C₆-C₁₀ aryl, which is optionally substituted with 1-4 R^{a};
(x) divalent heteroaryl of 5-10 ring atoms, wherein 1-4 ring atoms are heteroatoms, each independently selected from the group consisting of N, N(H), N(R^{c}), O, and S(O)₀₋₂; and which is optionally substituted with 1-4 R^{a}; and
(xi) divalent heterocyclyl of 3-10 ring atoms, wherein 1-3 ring atoms are heteroatoms, each independently selected from the group consisting of N, N(H), N(R^{c}), N(O⁻), O, and S(O)₀₋₂, and which is optionally substituted with 1-4 R^{a}.

10. The compound of any one of claims 1-7 and 9, wherein R⁶² is C₁-C₁₆ alkylene, which is optionally substituted with 1-2 substituents independently selected from halogen and C₃-C₆ cycloalkyl; and wherein 1-8 alkylene units are each optionally replaced with a group independently selected from the group consisting of:
(i) —O—;
(ii) —NH—;
(iii) —N(C₁-C₆ alkyl)-;
(iv) —C(O)—;
(v) —S—;
(vi) —S(O)—;
(vii) —S(O)₂—;
(viii) divalent C₃-C₁₀ cycloalkyl, which is optionally substituted with 1-4 R^{a};
(ix) divalent phenyl, which is optionally substituted with 1-4 R^{a};
(x) divalent heteroaryl of 5-10 ring atoms, wherein 1-4 ring atoms are heteroatoms, each independently selected from the group consisting of N, N(H), N(R^{c}), O, and S(O)₀₋₂; and which is optionally substituted with 1-4 R^{a}; and
(xi) divalent heterocyclyl of 3-10 ring atoms, wherein 1-3 ring atoms are heteroatoms, each independently selected from the group consisting of N, N(H), N(R^{c}), N(O⁻), O, and S(O)₀₋₂, and which is optionally substituted with 1-4 R^{a}.

11. The compound of any one of claims 1-7, 9, and 10, wherein R⁶² is C₁-C₈ alkylene, which is optionally substituted with 1-2 substituents independently selected from halogen and C₃-C₆ cycloalkyl; and wherein 1-6 alkylene units are each optionally replaced with a group independently selected from the group consisting of:
(i) —O—;
(ii) —NH—;
(iii) —N(C₁-C₆ alkyl)-;
(iv) —C(O)—;
(v) —S—;
(vi) —S(O)—;
(vii) —S(O)₂—;
(viii) divalent C₃-C₁₀ cycloalkyl, which is optionally substituted with 1-4 R^{a};
(ix) divalent phenyl, which is optionally substituted with 1-4 R^{a};
(x) divalent heteroaryl of 5-10 ring atoms, wherein 1-4 ring atoms are heteroatoms, each independently selected from the group consisting of N, N(H), N(R^{c}), O, and S(O)₀₋₂; and which is optionally substituted with 1-4 R^{a}; and
(xi) divalent heterocyclyl of 3-10 ring atoms, wherein 1-3 ring atoms are heteroatoms, each independently selected from the group consisting of N, N(H), N(R^{c}), N(O⁻), O, and S(O)₀₋₂, and which is optionally substituted with 1-4 R^{a}.

12. The compound of any one of claims 1-7 and 9-11, wherein R⁶² is C₁-C₈ alkylene, which is optionally substituted with 1-2 substituents independently selected from halogen and C₃-C₆ cycloalkyl; and wherein 1-6 alkylene units are each optionally replaced with a group independently selected from the group consisting of:
(i) —O—;
(ii) —NH—;
(iii) —N(C₁-C₆ alkyl)-;
(iv) —C(O)—;
(viii) divalent C₃-C₁₀ cycloalkyl, which is optionally substituted with 1-4 R^{a};
(ix) divalent phenyl, which is optionally substituted with 1-4 R^{a}; and
(xi) divalent heterocyclyl, wherein 1-3 ring atoms are heteroatoms, each independently selected from the group consisting of N, N(H), N(R^{c}), N(O⁻), O, and S(O)₀₋₂, and which is optionally substituted with 1-4 R^{a}.

13. The compound of any one of claims 1-7 and 9-12, wherein R⁶² has formula (II): 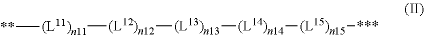
wherein ** indicates the point of attachment of formula (II) to R⁶¹, and *** indicates the point of attachment of formula (II) to the reactive group;
wherein each of n₁₁, n₁₂, n₁₃, n₁₄, and n₁₅ is independently 0 or 1, provided that at least one of n₁₁, n₁₂, n₁₃, n₁₄, and n₁₅ is 1; and
each occurrence of L¹¹, L¹², L¹³, L¹⁴, and L¹⁵ is independently selected from the group consisting of:
(i) —O—;
(ii) —NH—;
(iii) —N(C₁-C₆ alkyl)-;
(iv) —C(O)—;
(v) —S—;
(vi) —S(O)—:
(vii) —S(O)₂—;
(viii) divalent C₃-C₁₀ cycloalkyl, which is optionally substituted with 1-4 R^{a};
(ix) divalent phenyl, which is optionally substituted with 1-4 R^{a};
(x) divalent heteroaryl of 5-10 ring atoms, wherein 1-4 ring atoms are heteroatoms, each independently selected from the group consisting of N, N(H), N(R^{c}), O, and S(O)₀₋₂; and which is optionally substituted with 1-4 R^{a};
(xi) divalent heterocyclyl of 3-10 ring atoms, wherein 1-3 ring atoms are heteroatoms, each independently selected from the group consisting of N, N(H), N(R^{c}), N(O⁻), O, and S(O)₀₋₂, and which is optionally substituted with 1-4 R^{a}; and
(xi) C₁-C₂ alkylene.

14. The compound of claim 13, wherein each occurrence of L¹¹, L¹², L¹³, L¹⁴, and L¹⁵ is independently selected from the group consisting of:
(i) —O—;
(ii) —NH—;
(iii) —N(C₁-C₆ alkyl)-;
(iv) —C(O)—;
(viii) divalent C₃-C₁₀ cycloalkyl, which is optionally substituted with 1-4 R^{a};
(ix) divalent phenyl, which is optionally substituted with 1-4 R^{a};
(xi) divalent heterocyclyl of 3-10 ring atoms, wherein 1-3 ring atoms are heteroatoms, each independently selected from the group consisting of N, N(H), N(R^{c}), N(O⁻), O, and S(O)₀₋₂, and which is optionally substituted with 1-4 R^{a}; and
(xi) C₁-C₂ alkylene.

15. The compound of claim 13 or 14, wherein n₁₁ is 1.

16. The compound of any one of claims 13-15, wherein L¹¹ is —NH—.

17. The compound of any one of claims 13-15, wherein L¹¹ is —O—.

18. The compound of any one of claims 13-15, wherein L¹¹ is —CH₂—.

19. The compound of any one of claims 13-15, wherein L¹¹ is —C(O)—.

20. The compound of any one of claims 13-19, wherein n₁₅ is 1.

21. The compound of any one of claims 13-20, wherein L¹⁵ is divalent phenyl, which is optionally substituted with 1-4 R^{a}.

22. The compound of any one of claims 13-21, wherein L¹⁵ is unsubstituted divalent phenyl.

23. The compound of any one of claims 13-22, wherein one of n₁₂, n₁₃, and n₁₄ is 1, and the others are 0.

24. The compound of any one of claims 13-22, wherein two of n₁₂, n₁₃, and n₁₄ are 1, and the other is 0.

25. The compound of any one of claims 13-22, wherein each of n₁₂, n₁₃, and n₁₄ is 1.

26. The compound of any one of claims 13-22, wherein each of n₁₂, n₁₃, and n₁₄ is 0.

27. The compound of any one of claims 13-25, wherein each of L¹², L¹³, and L¹⁴ is independently selected from the group consisting of:
(iv) —C(O)—;
(viii) divalent C₃-C₁₀ cycloalkyl, which is optionally substituted with 1-4 R^{a};
(ix) divalent phenyl, which is optionally substituted with 1-4 R^{a};
(xi) divalent heterocyclyl of 3-10 ring atoms, wherein 1-3 ring atoms are heteroatoms, each independently selected from the group consisting of N, N(H), N(R^{c}), N(O⁻), O, and S(O)₀₋₂, and which is optionally substituted with 1-4 R^{a}; and
(xi) C₁-C₂ alkylene.

28. The compound of any one of claims 13-25 and 27, wherein each of L¹², L¹³, and L¹⁴, when present, is independently selected from the group consisting of —C(O)—, divalent cyclohexyl, and divalent piperidinyl.

29. The compound of claim 13 or 14, wherein nit is 1, and n₁₅ is 1.

30. The compound of claim 29, wherein L¹¹ is —NH—.

31. The compound of claim 29, wherein L¹¹ is —O—.

32. The compound of claim 29, wherein L¹¹ is —CH₂—.

33. The compound of claim 29, wherein L¹¹ is —C(O)—.

34. The compound of any one of claims 29-33, wherein L¹⁵ is divalent phenyl, which is optionally substituted with 1-4 R^{a}.

35. The compound of any one of claims 29-34, wherein L¹⁵ is unsubstituted divalent phenyl.

36. The compound of any one of claims 29-35, wherein one, two, or three of n₁₂, n₁₃, and n₁₄ are 1, and the others are 0.

37. The compound of any one of claims 29-34, wherein each of n₁₂, n₁₃, and n₁₄ is 0.

38. The compound of any one of claims 29-36, wherein each of L¹², L¹³, and L¹⁴ is independently selected from the group consisting of:
(iv) —C(O)—;
(viii) divalent C₃-C₁₀ cycloalkyl, which is optionally substituted with 1-4 R^{a};
(ix) divalent phenyl, which is optionally substituted with 1-4 R^{a};
(xi) divalent heterocyclyl of 3-10 ring atoms, wherein 1-3 ring atoms are heteroatoms, each independently selected from the group consisting of N, N(H), N(R′), N(O⁻), O, and S(O)₀₋₂, and which is optionally substituted with 1-4 R^{a}; and
(xi) C₁-C₂ alkylene.

39. The compound of any one of claims 29-36 and 38, wherein each of L¹², L¹³, and L¹⁴ is independently selected from the group consisting of —C(O)—, divalent cyclohexyl, and divalent piperidinyl.

40. The compound of any one of claims 1-39, wherein R⁶³ is 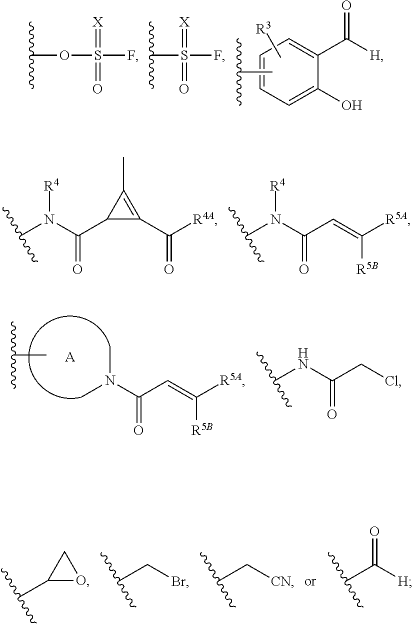
X is O or NR^{X}; and R^{X} is hydrogen, C₁-C6 alkyl, or C3-C6 cycloalkyl.

41. The compound of any one of claims 1-39, wherein R⁶³ is 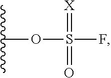
wherein X is O.

42. The compound of any one of claims 1-41, wherein Ring A is C₆₋₁₄ aryl optionally substituted with 1-4 substituents independently selected from the group consisting of oxo, R^{a}, and R^{b}.

43. The compound of any one of claims 1-42, wherein Ring A is phenyl optionally substituted with 1-4 substituents independently selected from the group consisting of oxo, R^{a}, and R^{b}.

44. The compound of any one of claims 1-43, wherein each of R¹ and R² is independently C₃₋₁₂ cycloalkyl optionally substituted with substituents independently selected from the group consisting of oxo, R^{a}, and R^{b}, optionally wherein each of R¹ and R² is independently C₅₋₇ cycloalkyl optionally substituted with substituents independently selected from the group consisting of oxo, R^{a}, and R^{b}.

45. The compound of any one of claims 1-44, wherein each of R¹ and R² is independently C₆ cycloalkyl optionally substituted with substituents independently selected from the group consisting of oxo, R^{a}, and R^{b}, optionally wherein each of R³ and R⁴ is independently C₁₋₁₀ alkyl optionally substituted with 1-4 independently selected R^{d}.

46. The compound of any one of claims 1-45, wherein each of R³ and R⁴ is CH₃.

47. The compound of any one of claims 1-46, wherein each of R⁵ is chloro.

48. A composition comprising a thiol-containing biomolecule and a compound of Formula (I).

49. A composition comprising a biomolecule comprising at least one cysteine residue and a compound of Formula (I).

50. A method of preparing a thiol-aryl conjugated biomolecule, the method comprising contacting a compound of Formula (I) with a biomolecule comprising at least one thiol under conditions sufficient to prepare the thiol-aryl conjugated biomolecule.

51. A method of preparing a cysteine-aryl conjugated biomolecule, the method comprising contacting a compound of Formula (I) with a biomolecule comprising at least one cysteine under conditions sufficient to prepare the cysteine-aryl conjugated biomolecule.

52. A method of preparing a gold(III) aryl complex comprising contacting a compound of Formula (I) and an aryl halide under conditions sufficient to prepare the gold(III) aryl complex.

53. The method of claim 51, wherein the aryl halide is an aryl iodide.

54. The compound of claim 1, wherein the compound is selected from the group of compounds recited in Tables disclosed herein.

55. A protein comprising an antigen-binding domain, wherein the antigen-binding domain comprising an oxime, the oxime having the structure: 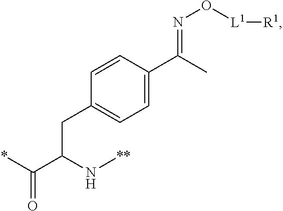
wherein:
* and ** represent the points of connection of the oxime to the antigen-binding domain;
L¹ is a bond or a C₁-C₆ alkylene optionally substituted with 1-2 substituents independently selected from halogen and C₃-C₆ cycloalkyl, and wherein 1-2 alkylene units are optionally replaced by O, N, C₃-C₆ cycloalkyl, or phenyl;
R¹ is azido, tetrazinyl, a C2-C3 alkyne, or an optionally substituted C8-C12 cycloalkyne.

56. The protein of claim 55, wherein the oxime is connected to the antigen-binding domain via an L amino acid.

57. The protein of claim 55, wherein the oxime is connected to the antigen-binding domain via a D amino acid.

58. The protein of any one of claims 55-57, wherein L¹ is a C1-C6 alkylene optionally substituted with 1-2 substituents independently selected from halogen and C3-C6 cycloalkyl, and wherein 1-2 alkylene units are optionally replaced by O, N, C3-C6 cycloalkyl, or phenyl.

59. The protein of any one of claims 55-58, wherein L¹ is a C1-C6 alkylene substituted with 1-2 substituents independently selected from halogen and C3-C6 cycloalkyl.

60. The protein of any one of claims 55-58, wherein L¹ is a C1-C6 alkylene wherein 1-2 alkylene units are replaced by O, N, C3-C6 cycloalkyl, or phenyl.

61. The protein of any one of claims 55-58, wherein L¹ is a C1-C6 alkylene.

62. The protein of any one of claims 55-58 or 61, wherein L¹ is methylene or ethylene.

63. The protein of any one of claims 55-58 or 61, wherein L¹ is n-propylene or isopropylene.

64. The protein of any one of claims 55-58 or 60, wherein L¹ is a C4-C6 cycloalkyl.

65. The protein of any one of claims 55-58, 60, or 64 wherein L¹ is

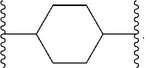 [SMILES: *C1CCC(*)CC1]

66. The protein of any one of claims 55-58 or 60, wherein L¹ is a PEG unit.

67. The protein of any one of claims 55-58, wherein L¹ is 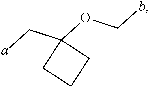
wherein “a” represents the point of connection of L¹ to the oxime and “b” represents the point of connection of L¹ to R¹.

68. The protein of any one of claims 55-57, wherein L¹ is a bond.

69. The protein of any one of claims 55-68, wherein R¹ is azido.

70. The protein of any one of claims 55-68, wherein R¹ is tetrazinyl.

71. The protein of any one of claims 55-68, wherein R¹ is a C2-C3 alkyne.

72. The protein of any one of claims 55-68, wherein R¹ is an optionally substituted C8-C12 cycloalkyne.

73. The protein of any one of claims 55-68 or 72, wherein R¹ is a C8-C12 cycloalkyne.

74. The protein of any one of claims 55-68 or 72-73, wherein R¹ is

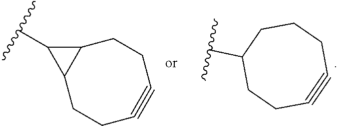 [SMILES: *C1C2CCC#CCCC12 ; *C1CCC#CCCC1]

75. A protein comprising an antigen-binding domain, wherein the antigen-binding domain comprises a modified phenylalanine residue, the modified phenylalanine residue having the structure: 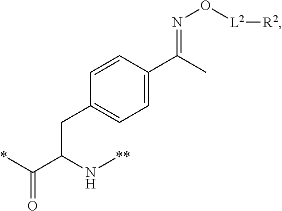
wherein:
* and ** represent the points of connection of the modified phenylalanine residue to the antigen-binding domain;
L² is a C2-C16 alkylene optionally substituted with 1-2 substituents independently selected from halogen and C3-C6 cycloalkyl, and wherein 1-6 alkylene units are optionally replaced by O, N, S, C3-C10 cycloalkyl, phenyl, 5-12 membered heteroaryl, and 4-14 membered heterocyclyl;
R² is 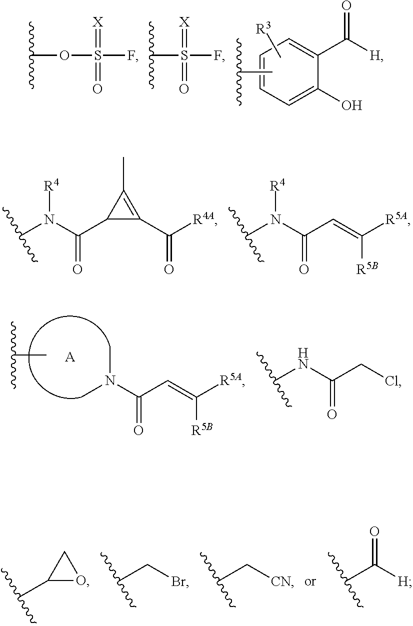
X is O or NR^{X};
R^{X} is hydrogen, C1-C6 alkyl, or C3-C6 cycloalkyl;
R³ is halogen or C1-C6 alkyl;
R⁴ is hydrogen or C1-C6 alkyl;
R^{4A} is C1-C6 alkyl or C3-C6 cycloalkyl;
R^{5A} and R^{5B} are independently hydrogen, halogen, or C1-C6 alkyl; or
R^{5A} and R^{5B} together with the carbon atom to which they are attached form a cyclopropyl;
Ring A is a 4-10 membered heterocyclyl.

76. The protein of claim 75, wherein the modified phenylalanine residue is an L modified phenylalanine residue.

77. The protein of claim 75, wherein the modified phenylalanine residue is a D modified phenylalanine residue.

78. The protein of any one of claims 75-77, wherein the modified phenylalanine residue is present in a CDR of the antigen-binding domain.

79. The protein of claim 78, wherein the CDR is a heavy chain CDR.

80. The protein of claim 78, wherein the CDR is a light chain CDR.

81. The protein of any one of claims 75-77, wherein the modified phenylalanine residue is present in a framework region of the antigen-binding domain.

82. A protein comprising an antigen-binding domain, wherein the antigen-binding domain comprises a modified cysteine residue, the modified cysteine residue having the structure: 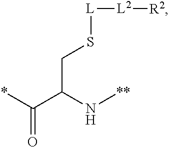
wherein:
* and ** represent the points of connection of the modified cysteine residue to the antigen-binding domain;
L is a bond, 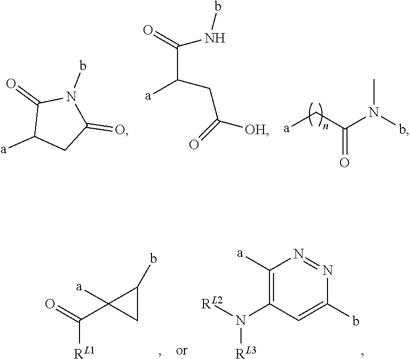
wherein a represents the point of connection of L to the sulfur atom of the modified cysteine residue and “b” represents the point of connection of L to L²;
n is 1 or 2;
R^{L1}, R^{L2}, and R^{L3}, are each independently selected C1-C10 alkyl;
L² is a C2-C16 alkylene optionally substituted with 1-2 substituents independently selected from halogen and C3-C6 cycloalkyl, and wherein 1-6 alkylene units are optionally replaced by O, N, S, C3-C10 cycloalkyl, phenyl, 5-12 membered heteroaryl, and 4-14 membered heterocyclyl;
R² is 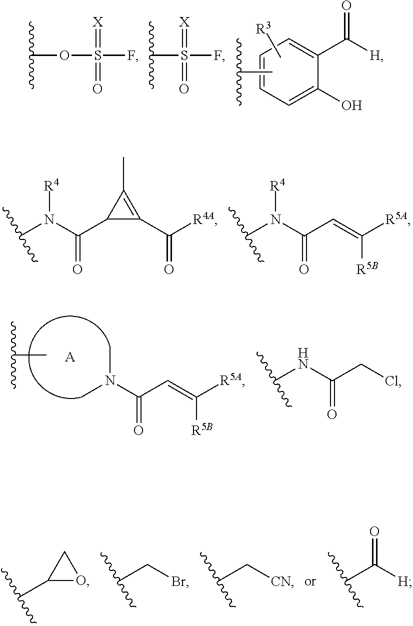
X is O or NR^{X};
R^{X} is hydrogen, C1-C6 alkyl, or C3-C6 cycloalkyl;
R³ is halogen or C1-C6 alkyl;
R⁴ is hydrogen or C1-C6 alkyl;
R^{4A} is C1-C6 alkyl or C3-C6 cycloalkyl;
R^{5A} and R^{5B} are independently hydrogen, halogen, or C1-C6 alkyl; or
R^{5A} and R^{5B} together with the carbon atom to which they are attached form a cyclopropyl;
Ring A is a 4-10 membered heterocyclyl.

83. The protein of claim 82, wherein the modified cysteine residue is an L modified cysteine residue.

84. The protein of claim 82, wherein the modified cysteine residue is a D modified cysteine residue.

85. The protein of any one of claims 82-84, wherein the modified cystine residue is present in a CDR of the antigen-binding domain.

86. The protein of claim 85, wherein the CDR is a heavy chain CDR.

87. The protein of claim 85, wherein the CDR is a light chain CDR.

88. The protein of any one of claims 82-84, wherein the modified cystine residue is present in a framework region of the antigen-binding domain.

89. The protein of any one of claims 82-88, wherein L is 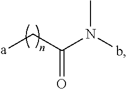
wherein “a” represents the point of connection of L to the sulfur atom of the modified cysteine residue and “b” represents the point of connection of L to L².

90. The protein of any one of claims 82-89, wherein n is 1.

91. The protein of any one of claims 82-89, wherein n is 2.

92. The protein of any one of claims 82-88, wherein L is 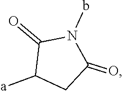
wherein “a” represents the point of connection of L to the sulfur atom of the modified cysteine residue and “b” represents the point of connection of L to L².

93. The protein of any one of claims 82-88, wherein L is 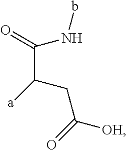
wherein “a” represents the point of connection of L to the sulfur atom of the modified cysteine residue and “b” represents the point of connection of L to L².

94. The protein of any one of claims 82-88, wherein L is R^{L1} 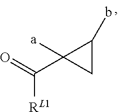
wherein “a” represents the point of connection of L to the sulfur atom of the modified cysteine residue and “b” represents the point of connection of L to L².

95. The protein of any one of claims 82-88 or 93, wherein R^{L1} is a C1-C6 alkyl.

96. The protein of any one of claims 82-88 or 93-94, wherein R^{L1} is ethyl.

97. The protein of any one of claims 82-88, wherein L is 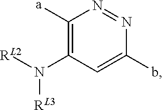
wherein “a” represents the point of connection of L to the sulfur atom of the modified cysteine residue and “b” represents the point of connection of L to L².

98. The protein of any one of claims 82-88 or 97, wherein R^{L2} and R^{L3} are independently selected C1-C6 alkyl.

99. The protein of any one of claims 82-88 or 96-97, wherein R^{L2} and R^{L3} are each methyl.

100. The protein of any one of claims 82-88, wherein L is a bond.

101. The protein of any one of claims 75-93, wherein L² is a C2-C16 alkylene optionally substituted with 1-2 substituents independently selected from halogen and C3-C6 cycloalkyl.

102. The protein of any one of claims 75-93, wherein L² is a C2-C16 alkylene wherein 1-6 alkylene units are optionally replaced by O, N, S, C3-C10 cycloalkyl, phenyl, 5-12 membered heteroaryl, and 4-14 membered heterocyclyl.

103. The protein of any one of claims 75-93, wherein L² is a C2-C16 alkylene substituted with 1-2 substituents independently selected from halogen and C3-C6 cycloalkyl, and wherein 1-6 alkylene units are optionally replaced by O, N, S, C3-C10 cycloalkyl, phenyl, 5-12 membered heteroaryl, and 4-14 membered heterocyclyl.

104. The protein of any one of claims 75-93, wherein L² is a C2-C16 alkylene optionally substituted with 1-2 substituents independently selected from halogen and C3-C6 cycloalkyl, and wherein 1-6 alkylene units are replaced by O, N, S, C3-C10 cycloalkyl, phenyl, 5-12 membered heteroaryl, and 4-14 membered heterocyclyl.

105. The protein of any one of claims 75-93, wherein L² is a C2-C16 alkylene substituted with 1-2 substituents independently selected from halogen and C3-C6 cycloalkyl, and wherein 1-6 alkylene units are replaced by O, N, S, C3-C10 cycloalkyl, phenyl, 5-12 membered heteroaryl, and 4-14 membered heterocyclyl.

106. The protein of any one of claims 75-100 or 102-105, wherein one alkylene unit of L² is replaced by a 5-12 membered heteroaryl.

107. The protein of claim 106, wherein the 5-12 membered heteroaryl is a 5-6 membered heteroaryl.

108. The protein of claim 106 or 107, wherein the 5-12 membered heteroaryl is a 6 membered heteroaryl.

109. The protein of any one of claims 106-108 wherein the 5-12 membered heteroaryl is pyridinyl, pyrimidinyl, or pyrazinyl.

110. The protein of any one of claims 75-95, wherein L² is a C2-C16 alkylene.

111. The protein of any one of claims 75-95 or 99, wherein L² is a C2-C6 alkylene.

112. The protein of any one of claims 75-93 or 95-98, wherein L² comprises one triazole ring.

113. The protein of any one of claims 75-93, wherein L² is selected from the group consisting of: 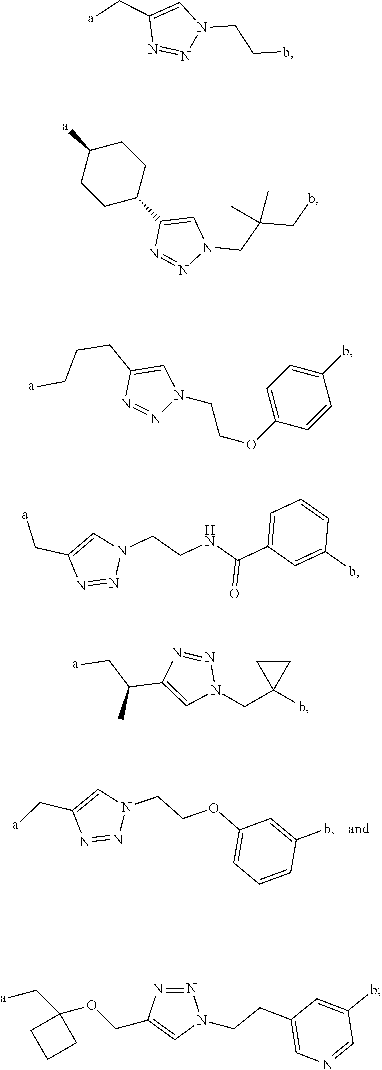
wherein “a” represents the point of connection of L² to L or to the modified phenylalanine residue or to the modified cysteine residue and “b” represents the point of connection of L² to R².

114. The protein of any one of claims 75-93, wherein L² is selected from the group consisting of: 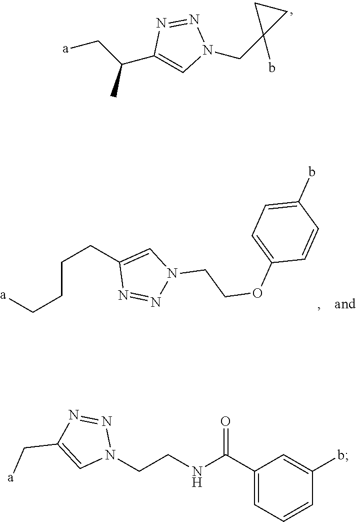
wherein “a” represents the point of connection of L² to L or to the modified phenylalanine residue or to the modified cysteine residue and “b” represents the point of connection of L² to R².

115. The protein of any one of claims 75-93, wherein L² is selected from the group consisting of: 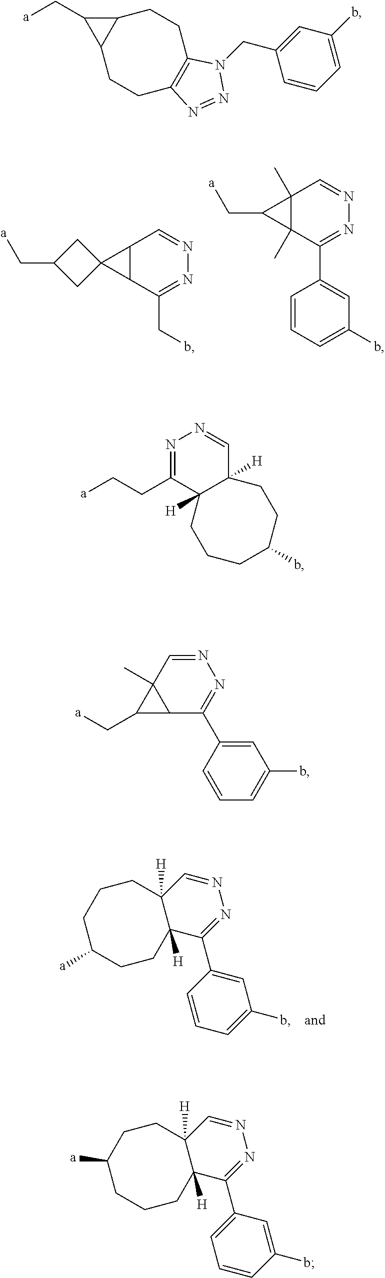
wherein “a” represents the point of connection of L² to L or to the modified phenylalanine residue or to the modified cysteine residue and “b” represents the point of connection of L² to R².

116. The protein of any one of claims 75-93, wherein L² is 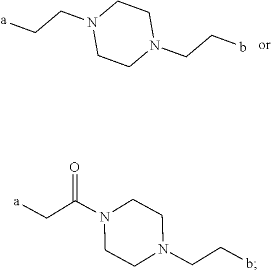
wherein “a” represents the point of connection of L² to L or to the modified phenylalanine residue or to the modified cysteine residue and “b” represents the point of connection of L² to R².

117. The protein of any one of claims 75-116, wherein R² is 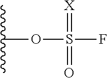
or —O—SO₂—HET, optionally R² is 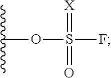
optionally R² is —O—SO₂—HET.

118. The protein of claims 75-117, wherein X is NR^{X}.

119. The protein of any one of claims 75-118, wherein R^{X} is hydrogen.

120. The protein of any one of claims 75-118, wherein R^{X} is C1-C6 alkyl.

121. The protein of any one of claims 75-118 or 120, wherein R^{X} is methyl.

122. The protein of any one of claims 75-118, wherein R^{X} is C3-C6 cycloalkyl.

123. The protein of any one of claims 75-118 or 122, wherein R^{X} is cyclopropyl.

124. The protein of claims 75-117, wherein X is O.

125. The protein of any one of claims 75-116, wherein R² is

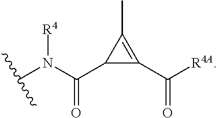

126. The protein of any one of claims 75-116 or 125, wherein R^{4A} is C1-C6 alkyl.

127. The protein of any one of claims 75-116 or 125-126, wherein R^{4A} is methyl.

128. The protein of any one of claims 75-116 or 125, wherein R^{4A} is C3-C6 cycloalkyl.

129. The protein ofany one of claims 75-116, wherein R² is

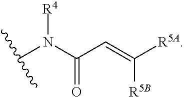

130. The protein of any one of claims 75-116 or 125-129, wherein R⁴ is C1-C6 alkyl.

131. The protein of any one of claims 75-116 or 125-130, wherein R⁴ is methyl.

132. The protein of any one of claims 75-116 or 125-129, wherein R⁴ is hydrogen.

133. The protein of any one of claims 75-116, wherein R² is

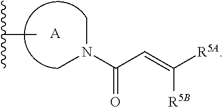

134. The protein of any one of claims 75-116 or 129-133, wherein R^{5A} and R^{5B} are independently hydrogen, halogen, or C1-C6 alkyl.

135. The protein of any one of claims 75-116 or 129-134, wherein R^{5A} and R^{5B} are each hydrogen.

136. The protein of any one of claims 75-116 or 129-134, wherein R^{5A} and R^{5B} are each independently C1-C6 alkyl.

137. The protein of any one of claims 75-116 or 129-134, wherein one of R^{5A} and R^{5B} is hydrogen and the other of R^{5A} and R^{5B} is C1-C6 alkyl.

138. The protein of any one of claims 75-116 or 129-134, wherein one of R^{5A} and R^{5B} is halogen and the other of R^{5A} and R^{5B} is hydrogen, halogen, or C1-C6 alkyl.

139. The protein of any one of claims 75-116 or 129-133, wherein R^{5A} and R^{5B} together with the carbon atom to which they are attached form a cyclopropyl.

140. The protein of any one of claims 75-105 or 133-139, wherein Ring A is a 4-10 membered heterocyclyl.

141. The protein of any one of claims 75-105 or 139-140, wherein Ring A is a 5-6 membered heterocyclyl.

142. The protein of any one of claims 75-105 or 139-141, wherein Ring A is piperidine or piperazine.

143. The protein of any one of claims 75-116, wherein R² is

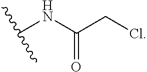 [SMILES: *NC(=O)CCl]

144. The protein of any one of claims 75-116, wherein R² is

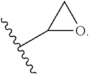 [SMILES: *C1CO1]

145. The protein ofany one of claims 75-116, wherein R² is

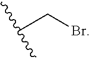 [SMILES: *CBr]

146. The protein of any one of claims 75-116, wherein R² is

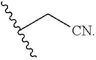 [SMILES: *CC#N]

147. The protein of any one of claims 75-116, wherein R² is

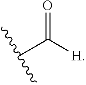

148. The protein of any one of claims 75-93, wherein L² is selected from the group consisting of: 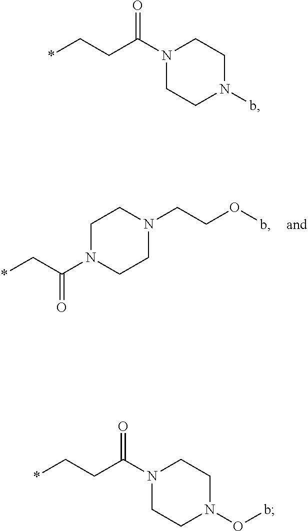
wherein “a” represents the point of connection of L² to L or to the modified phenylalanine residue or to the modified cysteine residue and “b” represents the point of connection of L² to R².

149. The protein ofany one of claims 75-116 or 148, wherein R² is

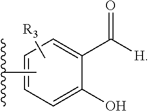

150. The protein of any one of claims 75-116 or 148-149, wherein R³ is halogen.

151. The protein of claims 75-116 or 148-149, wherein R³ is C1-C6 alkyl.

152. The protein of any one of claims 75-100, wherein -L²-R² is 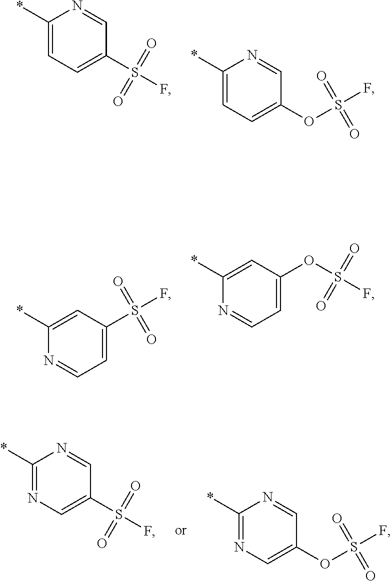
wherein * represents the point of connection of L² to L.

153. The protein of any one of claims 55-152, wherein the protein is an antibody.

154. The protein of claim 153, wherein the antibody is a human antibody, a humanized antibody, or a veneered antibody.

155. The protein of claim 153, wherein the antibody is a human IgG1, human IgG2, human IgG3, or human IgG4 antibody.

156. The protein of any one of claims 55-152, wherein the protein is or comprises a single chain Fv (scFv), a VHH, a VNAR, a DARpin, a single domain antibody (sdAb), an Adnectin/Centyrin, an Affibody, a Knottin, a bicyclic peptide, or a cyclic peptide.

157. The protein of any one of claims 55-156, wherein the protein further comprises a conjugated cytotoxic or cytostatic agent.

158. The protein of any one of claims 55-156, wherein the protein comprises a radioisotope.

159. The protein of any one of claims 55-158, wherein the antigen-binding domain specifically binds to a target protein.

160. The protein of claim 159, wherein the target protein comprises an extracellular domain, and the antigen-binding domain specifically binds to the extracellular domain.

161. A pharmaceutical composition comprising the protein of any one of claims 75-160 and at least one pharmaceutically acceptable excipient.

162. A kit comprising (a) the protein of any one of claims 75-160 and (b) a pharmaceutically acceptable excipient.

163. A kit comprising the pharmaceutical composition of claim 161 and instructions for administration of the pharmaceutical composition to a human subject.

164. A method of treating in a subject in need thereof, comprising administering to the subject a therapeutically effective amount of the protein of any one of claims 75-160, or the pharmaceutical composition of claim 161.

165. A method of inducing or increasing internalization of the protein into a mammalian cell that expresses the target protein comprising contacting the mammalian cell with the protein of claim 159 or 160.

166. The method of claim 165, wherein the mammalian cell is in vivo.

167. The method of claim 165, wherein the mammalian cell is in vitro.

168. A method of inhibiting the activity of the target protein in a mammalian cell, comprising contacting the target protein with the protein of claim 159 or 160.

169. A method of reducing the amount of the target protein in a mammalian cell comprising the target protein, the method comprising contacting the target protein with the protein of claim 159 or 160.

170. A method of inducing cell death in a mammalian cell comprising the target protein, the method comprising contacting the cell with the protein of claim 159 or 160.

171. The method of any one of claims 168-170, wherein the mammalian cell is in vivo.

172. The method of any one of claims 168-170, wherein the mammalian cell is in vitro.

173. A method of screening for a protein that forms a covalent bond with a target protein in a mammalian cell, the method comprising:
contacting the target protein with a protein of any one of claims 75-160; and
determining whether a covalent bond has been formed between the protein and the target protein.

174. The method of claim 173, wherein the method further comprises:
determining whether the mammalian cell has internalized the protein.

175. The method of claim 173, wherein the method further comprises:
determining whether the contacting has inhibited an activity of the target protein; and/or
determining whether the contacting has induced cell death of the mammalian cell.

176. A protein-protein conjugate comprising a first protein A and a second protein B, wherein the protein-protein conjugate has the structure: 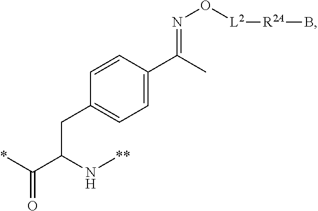
wherein the first protein A comprises an antigen-binding domain, wherein the antigen-binding domain comprises a modified phenylalanine residue, wherein:
* and ** represent the points of connection of the modified phenylalanine residue to the antigen-binding domain of the first protein A;
L² is a C2-C16 alkylene optionally substituted with 1-2 substituents independently selected from halogen and C3-C6 cycloalkyl, and wherein 1-6 alkylene units are optionally replaced by O, N, S, C3-C10 cycloalkyl, phenyl, 5-12 membered heteroaryl, and 4-14 membered heterocyclyl;
R^{2A} is 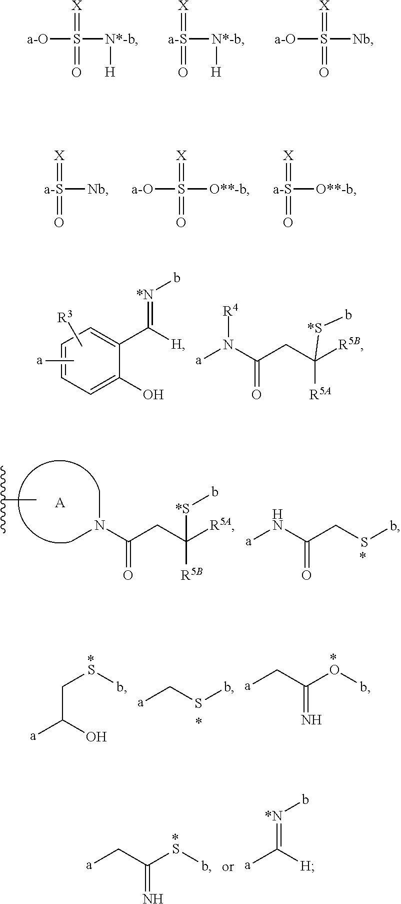
“a” represents the connection of R^{2A} to L², “b” represents the connection of R^{2A} to protein B, N* is a nitrogen atom of a lysine residue of protein B, S* is a sulfur atom of a cysteine residue of protein B, O* is an oxygen atom from a serine residue or a threonine residue of protein B, Nb is the nitrogen atom of a histidine residue of protein B and the connection of R^{2A} to protein B, and O** is an oxygen atom from a tyrosine residue of protein B;
X is O or NR^{X};
R^{X} is hydrogen, C1-C6 alkyl, or C3-C6 cycloalkyl;
R³ is halogen or C1-C6 alkyl;
R⁴ is hydrogen or C1-C6 alkyl;
R^{4A} is C1-C6 alkyl or C3-C6 cycloalkyl;
R^{5A} and R^{5B} are independently hydrogen, halogen, or C1-C6 alkyl; or
R^{5A} and R^{5B} together with the carbon atom to which they are attached form a cyclopropyl;
Ring A is a 4-10 membered heterocyclyl;
wherein the antigen-binding domain of the first protein A specifically binds to the second protein B.

177. The protein-protein conjugate of claim 176, wherein the modified phenylalanine residue is present in a CDR of the antigen-binding domain.

178. The protein-protein conjugate of claim 177, wherein the CDR is a heavy chain CDR.

179. The protein-protein conjugate of claim 177, wherein the CDR is a light chain CDR.

180. The protein-protein conjugate of claim 176, wherein the modified phenylalanine residue is present in a framework region of the antigen-binding domain.

181. A protein-protein conjugate comprising a first protein A and a second protein B, wherein the protein-protein conjugate has the structure: 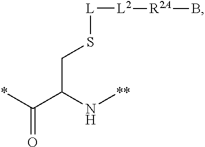
wherein the first protein A comprises an antigen-binding domain, wherein the antigen-binding domain comprises a modified cysteine residue, wherein:
* and ** represent the points of connection of the modified cysteine residue to the antigen-binding domain;
L is a bond, 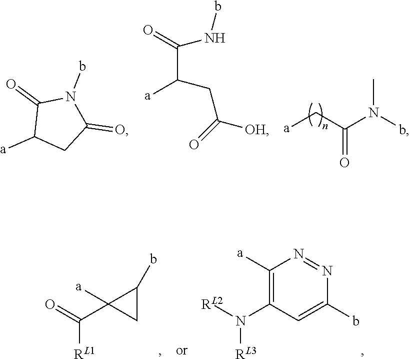
wherein “a” represents the point of connection of L to the sulfur atom of the modified cysteine residue and “b” represents the point of connection of L to L²;
n is 1 or 2;
R^{L1}, R^{L2}, and R^{L3}, are each independently selected C1-C10 alkyl;
L² is a C2-C16 alkylene optionally substituted with 1-2 substituents independently selected from halogen and C3-C6 cycloalkyl, and wherein 1-6 alkylene units are optionally replaced by O, N, S, C3-C10 cycloalkyl, phenyl, 5-12 membered heteroaryl, and 4-14 membered heterocyclyl;
R^{2A} is 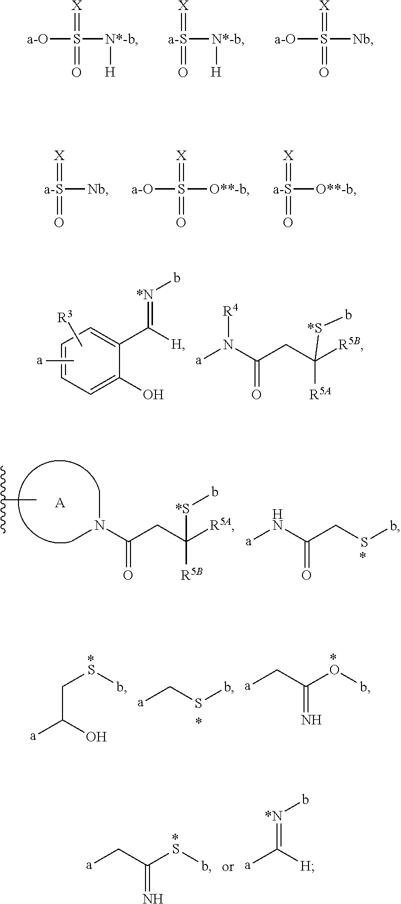
“a” represents the connection of R^{2A} to L², “b” represents the connection of R^{2A} to protein B, N* is a nitrogen atom of a lysine residue of protein B, S* is a sulfur atom of a cysteine residue of protein B, O* is an oxygen atom from a serine residue or a threonine residue of protein B, Nb is the nitrogen atom of a histidine residue of protein B and the connection of R^{2A} to protein B, and O** is an oxygen atom from a tyrosine residue of protein B;
X is O or NR^{X};
R^{X} is hydrogen, C1-C6 alkyl, or C3-C6 cycloalkyl;
R³ is halogen or C1-C6 alkyl;
R⁴ is hydrogen or C1-C6 alkyl;
R^{4A} is C1-C6 alkyl or C3-C6 cycloalkyl;
R^{5A} and R^{5B} are independently hydrogen, halogen, or C1-C6 alkyl; or
R^{5A} and R^{5B} together with the carbon atom to which they are attached form a cyclopropyl;
Ring A is a 4-10 membered heterocyclyl,
wherein the antigen-binding domain of the first protein A specifically binds to the second protein B.

182. The protein-protein conjugate of claim 181, wherein the modified cysteine residue is present in a CDR of the antigen-binding domain.

183. The protein-protein conjugate of claim 181, wherein the CDR is a heavy chain CDR.

184. The protein-protein conjugate of claim 181, wherein the CDR is a light chain CDR.

185. The protein-protein conjugate of claim 181, wherein the modified cysteine residue is present in a framework region of the antigen-binding domain.

186. The protein-protein conjugate of any one of claims 181-185, wherein the first protein A is an antibody.

187. The protein-protein conjugate of claim 186, wherein the antibody is a human antibody, a humanized antibody, or a veneered antibody.

188. The protein-protein conjugate of claim 186, wherein the antibody is a human IgG1, human IgG2, human IgG3, or human IgG4 antibody.

189. The protein-protein conjugate of any one of claims 181-188, wherein the first protein A is or comprises a single chain Fv (scFv), a VHH, a VNAR, a DARpin, a single domain antibody (sdAb), an Adnectin/Centyrin, an Affibody, a Knottin, a bicyclic peptide, or a cyclic peptide.

190. The protein-protein conjugate of any one of claims 181-189, wherein the first protein A further comprises a conjugated cytotoxic or cytostatic agent.

191. The protein-protein conjugate of any one of claims 181-189 wherein the first protein A comprises a radioisotope.

192. The protein-protein conjugate of claims 181-191, wherein the second protein B comprises an extracellular domain, and the antigen-binding domain specifically binds to the extracellular domain.

193. A method of making a protein comprising an antigen-binding domain, wherein the antigen-binding domain comprises a modified phenylalanine residue, the modified phenylalanine residue having the structure: 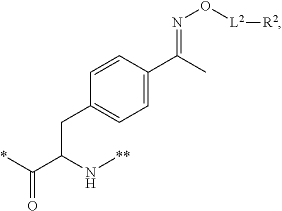
the method comprising contacting
(a) a compound having the structure Z—R² with
(b) a protein comprising an antigen-binding domain, wherein the antigen-binding domain comprising an oxime, the oxime having the structure: 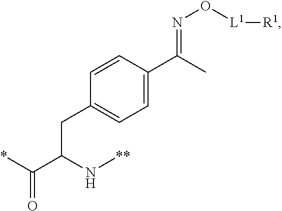
wherein:
Z reacts with -L¹- to form -L²-, wherein when R¹ is azido or tetrazinyl, then Z is a C2-C3 alkyne or an optionally substituted C8-C12 cycloalkyne, and when R¹ is a C2-C3 alkyne or an optionally substituted C8-C12 cycloalkyne, then Z is azido or tetrazinyl;
* and ** represent the points of connection of the oxime to the antigen-binding domain;
L² is a C2-C16 alkylene optionally substituted with 1-2 substituents independently selected from halogen and C3-C6 cycloalkyl, and wherein 1-6 alkylene units are optionally replaced by O, N, S, C3-C10 cycloalkyl, phenyl, 5-12 membered heteroaryl, and 4-14 membered heterocyclyl;
R² is 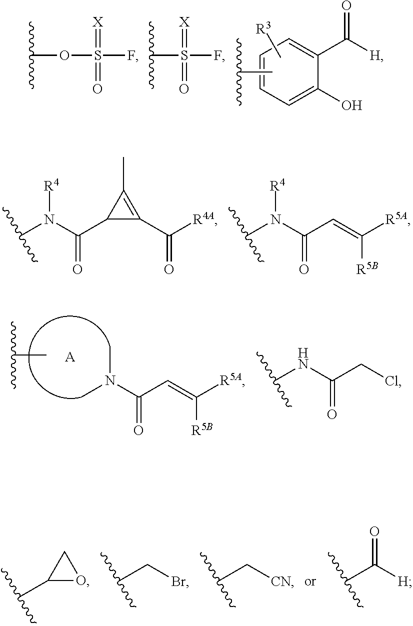
X is O or NR^{X};
R^{X} is hydrogen, C1-C6 alkyl, or C3-C6 cycloalkyl;
R³ is halogen or C1-C6 alkyl;
R⁴ is hydrogen or C1-C6 alkyl;
R^{4A} is C1-C6 alkyl or C3-C6 cycloalkyl;
R^{5A} and R^{5B} are independently hydrogen, halogen, or C1-C6 alkyl; or
R^{5A} and R^{5B} together with the carbon atom to which they are attached form a cyclopropyl;
Ring A is a 4-10 membered heterocyclyl;
L¹ is a bond or a C1-C6 alkylene optionally substituted with 1-2 substituents independently selected from halogen and C3-C6 cycloalkyl, and wherein 1-2 alkylene units are optionally replaced by O, N, C3-C6 cycloalkyl, or phenyl; and
R¹ is azido, tetrazinyl, a C2-C3 alkyne, or an optionally substituted C8-C12 cycloalkyne.

194. The method of claim 193, wherein the modified phenylalanine residue is present in a CDR of the antigen-binding domain.

195. The method of claim 194, wherein the CDR is a heavy chain CDR.

196. The method of claim 194, wherein the CDR is a light chain CDR.

197. The method of claim 139, wherein the modified phenylalanine residue is present in a framework region of the antigen-binding domain.

198. The method of any one of claims 193-197, wherein the protein is an antibody.

199. The method of claim 198, wherein the antibody is a human antibody, a humanized antibody, or a veneered antibody.

200. The method of claim 198, wherein the antibody is a human IgG1, human IgG2, human IgG3, or human IgG4 antibody.

201. The method of any one of claims 193-200, wherein the protein is or comprises a single chain Fv (scFv), a VHH, a VNAR, a DARpin, a single domain antibody (sdAb), an Adnectin/Centyrin, an Affibody, a Knottin, a bicyclic peptide, or a cyclic peptide.

202. The method of any one of claims 193-201, wherein the protein further comprises a conjugated cytotoxic or cytostatic agent.

203. The method of any one of claims 193-201, wherein the protein comprises a radioisotope.

204. The method of any one of claims 193-203, wherein the antigen-binding domain specifically binds to a target protein.

205. The protein of claim 204 wherein the target protein comprises an extracellular domain, and the antigen-binding domain specifically binds to the extracellular domain.


SEQUENCE LISTING (47 sequences)

SEQ ID NO: 1 - protein, 130 aa, synthetic construct
QVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSHHHHHH

SEQ ID NO: 2 - protein, 130 aa, synthetic construct
QVKLEESGGGSVQTGGSLRLTCAASGRTSRCYGMGWFRQAPGKEREFVSGISWRGDSTGYADSVKGRFTISRDNAKNTVDLQMNSLKPEDTAIYYCAAAAGSAWYGTLYEYDYWGQGTQVTVSSHHHHHH

SEQ ID NO: 3 - protein, 130 aa, synthetic construct
QVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLCEYDYWGQGTQVTVSSHHHHHH

SEQ ID NO: 4 - protein, 130 aa, synthetic construct; identical to SEQ ID NO: 2

SEQ ID NO: 5 - protein, 108 aa, synthetic construct
QVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTL

SEQ ID NO: 6 - protein, 108 aa, synthetic construct; identical to SEQ ID NO: 5

SEQ ID NO: 7 - protein, 108 aa, synthetic construct; identical to SEQ ID NO: 5

SEQ ID NO: 8 - protein, 108 aa, synthetic construct; identical to SEQ ID NO: 5

SEQ ID NO: 9 - protein, 108 aa, synthetic construct; identical to SEQ ID NO: 5

SEQ ID NO: 10 - protein, 108 aa, synthetic construct; identical to SEQ ID NO: 5

SEQ ID NO: 11 - protein, 108 aa, synthetic construct; identical to SEQ ID NO: 5

SEQ ID NO: 12 - protein, 108 aa, synthetic construct; identical to SEQ ID NO: 5

SEQ ID NO: 13 - protein, 108 aa, synthetic construct; identical to SEQ ID NO: 5

SEQ ID NO: 14 - protein, 108 aa, synthetic construct; identical to SEQ ID NO: 5

SEQ ID NO: 15 - protein, 108 aa, synthetic construct; identical to SEQ ID NO: 5

SEQ ID NO: 16 - protein, 108 aa, synthetic construct; identical to SEQ ID NO: 5

SEQ ID NO: 17 - protein, 108 aa, synthetic construct; identical to SEQ ID NO: 5

SEQ ID NO: 18 - protein, 108 aa, synthetic construct; identical to SEQ ID NO: 5

SEQ ID NO: 19 - protein, 108 aa, synthetic construct; identical to SEQ ID NO: 5

SEQ ID NO: 20 - protein, 108 aa, synthetic construct; identical to SEQ ID NO: 5

SEQ ID NO: 21 - protein, 108 aa, synthetic construct; identical to SEQ ID NO: 5

SEQ ID NO: 22 - protein, 108 aa, synthetic construct; identical to SEQ ID NO: 5

SEQ ID NO: 23 - protein, 108 aa, synthetic construct; identical to SEQ ID NO: 5

SEQ ID NO: 24 - protein, 30 aa, synthetic construct
QVKLEESGGGSVQTGGSLRLTCAASGRTSR

SEQ ID NO: 25 - protein, 30 aa, synthetic construct; identical to SEQ ID NO: 24

SEQ ID NO: 26 - protein, 30 aa, synthetic construct; identical to SEQ ID NO: 24

SEQ ID NO: 27 - protein, 30 aa, synthetic construct; identical to SEQ ID NO: 24

SEQ ID NO: 28 - protein, 30 aa, synthetic construct; identical to SEQ ID NO: 24

SEQ ID NO: 29 - protein, 30 aa, synthetic construct; identical to SEQ ID NO: 24

SEQ ID NO: 30 - protein, 30 aa, synthetic construct; identical to SEQ ID NO: 24

SEQ ID NO: 31 - protein, 30 aa, synthetic construct; identical to SEQ ID NO: 24

SEQ ID NO: 32 - protein, 30 aa, synthetic construct; identical to SEQ ID NO: 24

SEQ ID NO: 33 - protein, 30 aa, synthetic construct; identical to SEQ ID NO: 24

SEQ ID NO: 34 - protein, 30 aa, synthetic construct; identical to SEQ ID NO: 24

SEQ ID NO: 35 - protein, 30 aa, synthetic construct; identical to SEQ ID NO: 24

SEQ ID NO: 36 - protein, 30 aa, synthetic construct; identical to SEQ ID NO: 24

SEQ ID NO: 37 - protein, 30 aa, synthetic construct; identical to SEQ ID NO: 24

SEQ ID NO: 38 - protein, 30 aa, synthetic construct; identical to SEQ ID NO: 24

SEQ ID NO: 39 - protein, 30 aa, synthetic construct; identical to SEQ ID NO: 24

SEQ ID NO: 40 - protein, 30 aa, synthetic construct; identical to SEQ ID NO: 24

SEQ ID NO: 41 - protein, 30 aa, synthetic construct; identical to SEQ ID NO: 24

SEQ ID NO: 42 - protein, 30 aa, synthetic construct; identical to SEQ ID NO: 24

SEQ ID NO: 43 - protein, 30 aa, synthetic construct; identical to SEQ ID NO: 24

SEQ ID NO: 44 - protein, 30 aa, synthetic construct; identical to SEQ ID NO: 24

SEQ ID NO: 45 - protein, 30 aa, synthetic construct; identical to SEQ ID NO: 24

SEQ ID NO: 46 - protein, 21 aa, synthetic construct
EYDYWGQGTQVTVSSHHHHHH

SEQ ID NO: 47 - protein, 99 aa, synthetic construct
YGMGWFRQAPGKEREFVSGISWRGDSTGYADSVKGRFTISRDNAKNTVDLQMNSLKPEDTAIYYCAAAAGSAWYGTLYEYDYWGQGTQVTVSSHHHHHH